(19) United States
(12) Patent Application Publication
(10) Pub. No.: US 2025/0327054 A1
(43) Pub. Date: Oct. 23, 2025

(54) REPROGRAMMABLE ISRB NUCLEASES AND USES THEREOF

(71) Applicants: THE BROAD INSTITUTE, iNC., Cambridge, MA (US); MASSACHUSETTS INSTITUTE OF TECHNOLOGY, Cambridge, MA (US)

(72) Inventors: Feng Zhang, Cambridge, MA (US); Han Altae-Tran, Cambridge, MA (US); Soumya Kannan, Cambridge, MA (US)

(21) Appl. No.: 18/711,704

(22) PCT Filed: Nov. 22, 2022
(86) PCT No.: PCT/US2022/080340

§ 371 Date: May 20, 2024

Related U.S. Application Data
(60) Provisional application No. 63/282,575, filed on Nov. 23, 2021.
(60) Provisional application No. 63/351,659, filed on Jun. 13, 2022.

(51) Int. Cl. C12N 9/78; C12N 9/22; C12N 15/11; C12N 15/90

(52) U.S. Cl. CPC C12N 9/78; C12N 9/226; C12N 15/111; C12N 15/902; C12N 2310/20; C12Y 305/04004; C12Y 305/04005

(57) ABSTRACT

Systems, methods and compositions for targeting polynucleotides are detailed herein. In particular, engineered DNA-targeting systems comprising IsrB polypeptides, novel IsrB nucleases and reprogrammable targeting nucleic acid components and methods and application of use are provided.

### CROSS-REFERENCE TO RELATED APPLICATIONS

Reference is made to U.S. Provisional Application No. 63/282,575, filed Nov. 23, 2021; and U.S. Provisional Application No. 63/351,659, filed Jun. 13, 2022; the contents of which are incorporated by reference in their entireties herein.

### STATEMENT AS TO FEDERALLY SPONSORED RESEARCH

This invention was made with government support under Grant Nos. HL141201 and HG009761 awarded by The National Institutes of Health. The government has certain rights in the invention.

### REFERENCE TO AN ELECTRONIC SEQUENCE LISTING

This application contains a sequence listing filed in electronic form as an xml file entitled BROD-5480WP_ST26.xml, created on Nov. 21, 2022 and having size of 2,154,082 bytes. The contents of the electronic sequence listing are herein incorporated by reference in their entirety.

### TECHNICAL FIELD

The subject matter disclosed herein is generally directed to systems, methods and compositions used for targeted gene modification and nucleic acid editing utilizing systems comprising Isc polypeptides. In particular, the present disclosure provides DNA or RNA-targeting compositions comprising novel DNA or RNA-targeting nucleases and at least one targeting nucleic acid component.

### BACKGROUND

While there are genome-editing techniques available for producing targeted genome perturbations, there remains a pressing need for new and alternative genome engineering technologies that employ robust novel strategies and molecular mechanisms and are affordable, easy to set up, scalable, and amenable to targeting multiple positions within the genome. The CRISPR-Cas systems of bacterial and archaeal adaptive immunity are some such systems that show extreme diversity of protein composition and genomic loci architecture. These additional desirable tools in genome engineering and biotechnology would further advance the art.

Citation or identification of any document in this application is not an admission that such a document is available as prior art to the present invention.

### SUMMARY

In certain example embodiments, provided herein is a non-naturally occurring, engineered composition comprising a) an IsrB polypeptide comprising a split Ruv-C nuclease domain comprising RuvC-I, RuvC-II, and RuvC-III subdomains, and b) an ωRNA molecule comprising a scaffold and a reprogrammable spacer sequence, the ωRNA molecule capable of forming a complex with the IsrB polypeptide and directing the IsrB polypeptide to a target polynucleotide. In an embodiment, provided herein is a composition wherein the IsrB polypeptide comprises a PLMP domain (SEQ ID NO: 1524) and optionally a conserved C-terminal Y domain. In an embodiment, provided herein is a composition wherein the IsrB polypeptide comprises about 170 to about 700 amino acids. In an embodiment, provided herein is a composition wherein the reprogrammable spacer sequence comprises a spacer of 10 nucleotides (nt) to 150 nucleotides in length, preferably 12 to 50 nt, more preferably 15 and 45 nt in length.

In an embodiment, provided herein is a composition wherein the target sequence comprises a target adjacent motif (TAM) sequence 3′ of the target polynucleotide. In an embodiment, provided herein is a composition wherein the target polynucleotide is DNA.

In an embodiment, provided herein is a composition wherein the ωRNA further comprises an aptamer.

In an embodiment, provided herein is a composition wherein the ωRNA molecule further comprises an extension to add an RNA template.

In an embodiment, provided herein is a composition further comprising a functional domain associated with the IsrB protein.

In certain example embodiments, provided herein is a composition wherein the functional domain has transposase activity, methylase activity, demethylase activity, translation activation activity, translation repression activity, transcription activation activity, transcription repression activity, transcription release factor activity, chromatin modifying or remodeling activity, histone modification activity, nuclease activity, single-strand RNA cleavage activity, double-strand RNA cleavage activity, single-strand DNA cleavage activity, double-strand DNA cleavage activity, nucleic acid binding activity, detectable activity, or any combination thereof.

In a certain embodiment, provided herein is a composition further comprising a serine or tyrosine recombinase or integrase.

In an embodiment, provided herein is a composition further comprising a homologous recombination donor template comprising a donor sequence for insertion into a target polynucleotide.

In embodiments, provided herein is a vector system comprising one or more vectors encoding the IsrB polypeptide and the ωRNA molecule disclosed herein above.

In embodiments, provided herein is an engineered cell comprising the composition disclosed herein above.

In certain example embodiments, provided herein is a method of modifying a target polynucleotide sequence in a cell, comprising introducing to the cell the composition disclosed herein above. In an embodiment, is disclosed a method wherein the polypeptide and/or nucleic acid components are provided via one or more polynucleotides encoding the polypeptides and/or nucleic acid component(s), and wherein the one or more polynucleotides are operably configured to express the IsrB polypeptide and/or the ωRNA molecule. IN an embodiment, provided herein is a method wherein the modifying comprises cleaving a DNA polynucleotide.

In certain example embodiments, provided herein is a composition comprising an IsrB protein, wherein the IsrB protein comprises an N-terminal X domain, a RuvC domain, a Bridge Helix domain, and a C-terminal Y domain. In an embodiment, provided herein is a composition wherein the X domain is no more than 50 amino acids in length. In certain embodiments, provided herein is a composition wherein the IsrB protein is no more than 500, no more than 600, no more than 700, or no more than 800 amino acids in length. In an embodiment, provided herein is a composition wherein the Ruv-C domain of the IsrB protein is catalytically inactive. In an embodiment, provided herein is a composition wherein the nuclease domain has nickase activity or is engineered to have nickase activity.

In an embodiment, provided herein is a composition further comprising a homologous recombination donor template comprising a donor sequence for insertion into a target polynucleotide.

In embodiments, provided herein are one or more polynucleotides encoding one or more components of the composition as disclosed herein above.

In embodiments, provided herein are one or more vectors comprising the one or more polynucleotides as disclosed herein above.

In embodiments, provided herein is a cell or progeny thereof genetically engineered to express one or more components of the compositions as disclosed herein above.

In certain embodiments, provided herein is a method of targeting a polynucleotide comprising contacting a sample that comprises a target polynucleotide with the composition as disclosed herein above or the one or more polynucleotides or one or more vectors as disclosed herein above.

In certain embodiments, provided herein is a method wherein contacting results in modification of a gene product or modification of the amount or expression of a gene product.

In certain embodiments, provided herein is a method wherein the target sequence of the polynucleotide is a disease-associated target sequence.

In certain example embodiments, provided herein is an engineered, non-naturally occurring composition comprising: a) the IsrB protein as disclosed herein above, wherein the IscB protein is catalytically inactive, b) a nucleotide deaminase associated with or otherwise capable of forming a complex with the IsrB protein, and c) an ωRNA molecule capable of forming a complex with the IsrB protein and directing site-specific binding at a target sequence. In an embodiment, provided herein is a composition wherein the nucleotide deaminase is an adenosine deaminase or a cytidine deaminase. In embodiments, provided herein are polynucleotides encoding one or more components of the composition as disclosed herein above. In embodiments, provided herein are one or more vectors encoding the one or more polynucleotides as disclosed herein above.

In embodiments, provided herein is a cell or progeny thereof genetically engineered to express one or more components of the composition as disclosed herein above.

In certain example embodiments, provided herein is a method of editing nucleic acids in target polynucleotides comprising delivering the composition as disclosed herein above, the one or more polynucleotides as disclosed herein above, or one or more vectors as disclosed herein above to a cell or population of cells comprising the target polynucleotides. In an embodiment, provided herein is a method wherein the target polynucleotides are target sequences within genomic DNA. In an embodiment, provided herein is a method wherein the target polynucleotide is edited at one or more bases to introduce a G→A or C→T mutation. In an embodiment, provided herein is an isolated cell or progeny thereof comprising one or more base edits made using the method as disclosed herein above.

In certain example embodiments, provided herein is an engineered, non-naturally occurring composition comprising: a) the IsrB protein as disclosed herein above, wherein the IsrB is catalytically inactive, b) a reverse transcriptase associated with or otherwise capable of forming a complex with the IscrB protein, and c) ωRNA molecule capable of forming a complex with the IsrB protein and directing site-specific binding of the complex to a target sequence of a target polynucleotide, and further comprising a donor sequence for insertion into the target polynucleotide. In an embodiment, provided herein are one or more polynucleotides encoding one or more components of the composition as disclosed herein above. In an embodiment, provided herein are vectors encoding the one or more polynucleotides as disclosed herein above.

In certain example embodiments, provided herein is a method of modifying target polynucleotides comprising: delivering the composition as disclosed herein above, the one or more polynucleotides as disclosed herein above, or one or more vectors as disclosed herein above to a cell or population of cells comprising the target polynucleotides, wherein the complex directs the reverse transcriptase to the target sequence and the reverse transcriptase facilitates insertion of the donor sequence from the ωRNA molecule into the target polynucleotide.

In certain example embodiments, provided herein is a method wherein insertion of the donor sequence: a) introduces one or more base edits; b) corrects or introduces a premature stop codon; c) disrupts a splice site; d) inserts or restores a splice site; e) inserts a gene or gene fragment at one or both alleles of the target polynucleotide; or f) a combination thereof.

In an embodiment, provided herein is an isolated cell or progeny thereof comprising modifications made using the method as disclosed herein above.

In certain example embodiments, provided herein is an engineered, non-naturally occurring composition comprising: a) the IsrB protein as disclosed in Table 1; b) a non-LTR retrotransposon protein or integrase associated with or otherwise capable of forming a complex with the IsrB protein; c) ωRNA molecule capable of forming a complex with the IsrB protein and directing site-specific binding to a target sequence of a target polynucleotide; and d) a donor construct comprising a donor polynucleotide for insertion to the target polynucleotide and located between two binding elements capable of forming a complex with the non-LTR retrotransposon protein or integrase. In an embodiment, provided herein is a composition wherein the IsrB protein is fused to the N-terminus of the non-LTR retrotransposon protein or integrase. In an embodiment, provided herein is a composition wherein the IsrB protein has nickase activity.

In an embodiment, provided herein is a composition wherein the donor polynucleotide further comprises a polymerase processing element to facilitate 3′ end processing of the donor polynucleotide sequence.

In an embodiment, provided herein is a composition wherein the donor polynucleotide further comprises a homology region to the target sequence on the 5′ end of the donor construct, the 3′ end of the donor construct, or both.

In an embodiment, provided herein are one or more polynucleotides encoding one or more components of the composition as disclosed herein above.

In an embodiment, provided herein are one or more vectors comprising the one or more polynucleotides as disclosed herein above.

In a certain example embodiment, provided herein is a method of modifying target polynucleotides comprising: delivering the composition as disclosed herein above, the one or more polynucleotides as disclosed herein above, or the one or more vectors as disclosed herein above to a cell or population of cells comprising the target polynucleotides, wherein the complex directs the non-LTR retrotransposon protein to the target sequence and the non-LTR retrotransposon protein facilitates insertion of the donor polynucleotide sequence from the donor construct into the target polynucleotide.

In certain example embodiments, provided herein is a method wherein the insertion of the donor sequence: a) introduces one or more base edits; b) corrects or introduces a premature stop codon; c) disrupts a splice site; d) inserts or restores a splice site; e) inserts a gene or gene fragment at one or both alleles of the target polynucleotide; or; f) a combination thereof.

In an embodiment, provided herein is an isolated cell or progeny thereof comprising the modifications made using the method as disclosed herein above.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—PLMP Domain. Weblogo of PLMP domain found in IscB and IsrB proteins immediately upstream of the RuvC-I domain.

FIG. 2—Small RNA-seq of standalone ωRNAs in K. racemifer. Small RNA-seq reads greater than 200 bp mapped to standalone ωRNA loci in K. racemifer. 9 of the 10 loci contain an expressed ncRNA transcript corresponding to a guide and ωRNA scaffold. The ωRNA scaffold that is not expressed belongs to a group associated primarily with IsrB (G1c group—see FIG. 15).

FIG. 3—Complete RuvC/BH phylogenetic analysis with IQ Tree 2. Maximum likelihood phylogenetic analysis of all IsrB, IscB and Cas9 RuvC/BH domains using IQ Tree 2. The LG substitution model with Gamma rates with 4 categories was used with 5000 ultra fast bootstraps (with hill-climbing nearest neighbor change for each bootstrap tree). The tree was rooted on the IsrB family. Associations are calculated for each cluster based on non-redundant loci (at 90% sequence identity of the main locus ORF). Ga-Gi refer to the IscB/IsrB main ωRNA profiles in FIG. 15. HNH domain associations are shown with 3 colors, with cyan indicating the HNH domain has the H, N, and H catalytic residues, magenta indicating the HNH domain has the H, N, and N catalytic residues, and grey indicating that the HNH domain has an H, N, and not H/N catalytic residues. Sizes of REC-like insertion in the representative protein sequence for each cluster are shown as determined by the number of amino acids between the BH and the RuvC-II in the alignment. Total size of the representative protein sequence for each cluster is shown on the outer ring.

FIG. 4—Complete RuvC/BH/HNH phylogenetic (IQ Tree 2)×5000 Ufbs tree with associations. Maximum likelihood phylogenetic analysis of all IscB and Cas9 RuvC/BH/HNH domains using IQ Tree 2. The LG substitution model with Gamma rates with 4 categories was used with 5000 ultra fast bootstraps (with hill-climbing nearest neighbor change for each bootstrap tree). Tree is rooted using cluster 34777, which include some of the most ancestral IscBs as determined by the RuvC/BH phylogenetic analyses. Associations are calculated for each cluster based on non-redundant loci (at 90% sequence identity of the main locus ORF). Ga-Gi refer to the IscB/IsrB main ωRNA profiles in FIG. 13A. HNH domain associations are shown with 3 colors, with cyan indicating the HNH domain has the H, N, and H catalytic residues, magenta indicating the HNH domain has the H, N, and N catalytic residues, and grey indicating that the HNH domain has an H, N, and not H/N catalytic residues. Sizes of REC-like insertion in the representative protein sequence for each cluster are shown as determined by the number of amino acids between the BH and the RuvC-II in the alignment. Total size of the representative protein sequence

FIG. 5—Complete RuvC/BH/HNH phylogenetic (RaxML)×2000 bs. Maximum likelihood phylogenetic analysis of all IscB and Cas9 RuvC/BH/HNH domains using RaxML. The PROTGAMMALG model was used with 2000 rapid bootstraps. Tree is rooted using cluster 34777, which include some of the most ancestral IscBs as determined by the RuvC/BH phylogenetic analyses. Associations are calculated for each cluster based on non-redundant loci (at 90% sequence identity of the main locus ORF). Ga-Gi refer to the IscB/IsrB main ωRNA profiles in FIG. 15. HNH domain associations are shown with 3 shades of gray, with cyan indicating the HNH domain has the H, N, and H catalytic residues, magenta indicating the HNH domain has the H, N, and N catalytic residues, and grey indicating that the HNH domain has an H, N, and not H/N catalytic residues. Sizes of REC-like insertion in the representative protein sequence for each cluster are shown as determined by the number of amino acids between the BH and the RuvC-II in the alignment. Total size of the representative protein sequence for each cluster is shown on the outer ring.

FIG. 6—Complete RuvC/BH/HNH phylogenetic (mrbayes)×10M iterations. Bayesian phylogenetic analysis of IscB and early Cas9 RuvC/BH/HNH domains using MrBayes with random starting trees. The LG substitution model was used with Gamma rates with 4 categories. 4 independent runs were run with 16 chains per with a delta temperature of 0.025 per chain for a total of 10M generations. 1000 swaps were attempted each generation, and tree samples were collected every 50 generations. The average standard deviation of split frequencies was 0.057890 at the final generation. Associations are calculated for each cluster based on non-redundant loci (at 90% sequence identity of the main locus ORF). Ga-Gi refer to the IscB/IsrB main ωRNA profiles in FIG. 15. HNH domain associations are shown with 3 colors, with cyan indicating the HNH domain has the H, N, and H catalytic residues, magenta indicating the HNH domain has the H, N, and N catalytic residues, and grey indicating that the HNH domain has an H, N, and not H/N catalytic residues. Sizes of REC-like insertion in the representative protein sequence for each cluster are shown as determined by the number of amino acids between the BH and the RuvC-II in the alignment. Total size of the representative protein sequence for each cluster is shown on the outer ring.

FIG. 7—Complete RuvC/BH phylogenetic analysis with IQ Tree 2. Maximum likelihood phylogenetic analysis of all IsrB, IscB and Cas9 RuvC/BH domains using IQ Tree 2. The LG substitution model with Gamma rates with 4 categories was used with 5000 ultra fast bootstraps (with hill-climbing nearest neighbor change for each bootstrap tree). The tree was rooted on the IsrB family. Associations are calculated for each cluster based on non-redundant loci (at 90% sequence identity of the main locus ORF). Ga-Gi refer to the IscB/IsrB main ωRNA profiles in FIG. 40. HNH domain associations are shown with 3 colors, with cyan indicating the HNH domain has the H, N, and H catalytic residues, magenta indicating the HNH domain has the H, N, and N catalytic residues, and grey indicating that the HNH domain has an H, N, and not H/N catalytic residues. Sizes of REC-like insertion in the representative protein sequence for each cluster are shown as determined by the number of amino acids between the BH and the RuvC-II in the alignment. Total size of the representative protein sequence for each cluster is shown on the outer ring.

FIG. 8 IscB/IsrB ωRNA phylogenetic analysis focused on Cas9 evolution. Same phylogenetic tree is FIG. 39 but focused on the early Cas9 evolution with the CRISPR-associated IscB cluster 2089. Support values for each branching are shown above the branches. Not all clusters included in other phylogenetic analyses could not be included in this analysis due to lack of a completely alignable ωRNA. For example, clusters 57212 and 50962 were not included. Clusters 2964, 21041, 57212, and 50962 were inferred as ancestral relative to the CRISPR-associated IscB cluster 2089 for the RuvC/BH/HNH amino acid phylogenetic analyses with RaxML (FIG. 12).

FIG. 9A-9C—Diversity and evolution of IscB. (9A) Phylogenetic tree of IsrB, IscB and Cas9. Associations with IS200/605 TnpA, ωRNA, CRISPR arrays, anti-repeats (where applicable), and Cas acquisition genes. ORF size of cluster representative is shown on the outermost ring. Positions of evolutionary events described in (9A) are marked by grayed circles/squares. (9B) Inferred evolutionary timeline linking IsrB to Cas9 with exemplifying loci. (9C) Structural diversity and evolution of ωRNAs in IsrB and IscB systems.

FIG. 10A-10D-Sensitivity analysis for inferred Cas9 ancestor (10A) RaxML maximum likelihood phylogenetic tree of the RuvC/BH/HNH alignment with 2000 rapid boot straps for computing support values. Only sections of the tree relevant to the early evolution of Cas9 are shown. (10B) BLOSUM62 similarity comparison of the RuvC-I, RuvC-II, RuvC-III, and HNH core regions (with alignment trimming, alignments provided in supplementary file) for early Cas9 II-D (clusters Cas9_1261, Cas9_665, Cas9_1079), a typical Cas9 (cluster Cas9_758), the putative Cas9 ancestor (2089), and example IscBs. (10C-10D) random taxon dropout analysis using FastTree2. Sample size for each dropout percentage category was calculated such that each taxon is retained on average for 1000 bootstrap samples. Clusters 2089, Cas9_1079, Cas9_665, and Cas9_1261 were retained in all samples. Error bars were calculated using 2000 bootstraps from the final samples. (10C) proportion of trees supporting CRISPR-associated IscB 2089 as the direct ancestor of all Cas9s as a function of taxa dropout rate. (10D) proportion of trees supporting mono/paraphyletic topologies involving Cas9, IsrB, or early II-D Cas9s as a function of the taxa dropout rate.

FIG. 11 (SEQ ID NO: 1567-1595)—Comparison of early Cas9 tracrRNAs to conserved ωRNAs from IscB and IsrB. ωRNA from the putative ancestor of all Cas9s (2089) is shown as well. Conserved region shared by the tracrRNA and IscB/IsrB ωRNAs corresponds to the nexus pseudoknot hairpin. Alignment was generated using MAFFT-ginsi. Additional, less conserved regions are not shown for this alignment. Specifically, the 5′ end is not conserved between tracrRNA and IscB ωRNAs.

FIG. 12—IscB/IsrB ωRNA phylogenetic analysis using IQ Tree 2. Maximum likelihood phylogenetic tree inference for the DNA alignment of ωRNA from IscB/IsrBs using IQ Tree 2. This tree was built using the best likelihood scoring tree of 200 independent runs as the starting tree with 5000 ultra fast bootstraps (with hill-climbing nearest neighbor change for each bootstrap tree) under the GTR substitution model, using empirical DNA frequencies from the alignment, ascertainment bias correction, and Gamma rates with 4 categories.

FIG. 13A-13B—Diverse ωRNAs associated with isrB and iscB. Secondary structure predictions for the main groups of ωRNA scaffolds associated with iseBs and isrBs. (13A) G1a, G1d, G1e, G1f, G1g, and G1i are associated with iscB while (13B) G1b, G1c, G1h are associated with isrB. G1a, G1b, G1c, G1d, G1h, and G1i secondary structures were predicted using R-scape while G1e, G1f, G1g were computed using consensus secondary structures with ViennaRNA due to the smaller sample sizes. While pseudoknots were not identified de novo for G1e, G2f, G1g, potential pseudoknots in similar locations to the other iscB/isrB ωRNAs can be found. Guide locations for all iscB/isrB ωRNAs would be predicted to be immediately upstream from each ωRNA scaffold where the 5′ label is located.

FIG. 14A-14J—Exploration of the diversity of IS200/605 superfamily nucleases. (14A) Evolution between IS200/605 transposon superfamily-encoded nucleases and associated RNAs. Dashed lines reflect tentative/unknown relationships. (14B) Locations of IscB loci and fragments in the I. tetrasporus genome. Intact locus is labeled as “ChlorIscB.” (14C) Small RNA-seq of I. tetrasporus. (14D) Weblogo of ChlorIscB cleavage TAM using a reprogrammed guide in an IVTT TAM screen. (14E) Weblogo of OgeuIscB TAM using a reprogrammed guide in an IVTT TAM screen. (14F (SEQ ID NO: 1596-1604)) Targeted OgeuIscB mediated indel formation in HEK293FT cells ordered by abundance, with indel size on the left. (14G) OgeuIscB mediated indel formation at multiple sites in HEK293T cells (* indicates p<0.05). (14H) Native expression of IsrB ωRNA in K. racemifer. (14I) Weblogo of Desulfovigula thermocuniculi (DthIsrB) TAM using a reprogrammed guide in an IVTT TAM screen. (14J) DthIsrB mediates ωRNA-guided non-target strand nicking in a TAM- and target-dependent manner in an IVTT cleavage assay using 5′ strand-specific labeled targets.

FIG. 15—Small RNA-seq of IsrB loci from K. racemifer shows expressed associated ωRNAs. Small RNA-seq reads greater than 200 bp mapped to the 5 IsrB loci present in K. racemifer. Each locus contains an expressed ncRNA transcript corresponding to a guide and ωRNA scaffold upstream of the IsrB ORF.

FIG. 16A-16C—IsrB nicks dsDNA in a target and TAM-dependent manner. (16A) Target cleavage by DthIsrB at various temperatures from 40 C to 70 C at 5 C increments. All cleavage reactions were performed using RNP complexes produced by IVTT reactions for 1 hour at the indicated temperatures, run on denaturing PAGE gels, and imaged in the IR800 and IR700 channels. Optimal temperature for nicking activity is approximately 60 C. Additionally, double-stranded cleavage was not observed at any temperature. (16B) Target cleavage by DchIsrB at various temperatures from 30 C to 60 C at 5 C increments. All cleavage reactions were performed using NP complexes produced by IVTT reactions for 1 hour at the indicated temperatures for 1 hour at the indicated temperatures, run on denaturing PAGE gels, and imaged in the IR700 and IR800 channels. Optimal temperature for nicking activity is approximately 45° C. Double-stranded cleavage was not observed at any temperature. (16C) Target cleavage by DthIsrB, DchIsrB, and KraIscB-1 performed at optimal temperatures (60° C., 45° C., and 37° C. respectively). All cleavage reactions were performed using RNP complexes produced by IVTT and incubated for 1 hour at their respective temperatures. Products were run on native PAGe and denaturing PAGE gels and imaged in the IR800 and IR700 channels. DthIsrB and DchIsrB perform non-target strand dsDNA nicking with no detectable double-stranded cleavage compared to KraIscB1.

FIG. 17—Phylogenetic distribution. Distribution of IscB, IsrB, and Cas9 across archaeal and bacterial phyla. Heatmap displays percentages of genomes containing a specific system.

FIG. 18—Naturally-occurring RNA-guided DNA-targeting systems. Comparison of Ω2 (OMEGA) systems with other known RNA-guided systems. In contrast to CRISPR systems, which capture spacer sequences and store them within the CRISPR array, in the locus, Ω systems transpose their loci (or trans-acting loci) into target sequences, apparently, converting targets into ωRNA guides in a process that can be called guide conscription.

FIG. 19—Targets of IscB/IsrB guides. Same as FIG. 20A with results of target search mapped on the second outermost ring. Notable groups are shown as labeled arcs on the outermost ring.

FIG. 20A-20B—Complete RuvC/BH phylogenetic analysis. (20A) Maximum likelihood phylogenetic analysis of all IsrB, IscB and Cas9 RuvC/BH domains using IQ-Tree 2. The LG substitution model with Gamma rates with 4 categories was used with 5000 ultrafast bootstraps (with hill-climbing nearest neighbor change for each bootstrap tree). (20B) Maximum likelihood phylogenetic analysis of all IsrB, IscB and Cas9 RuvC/BH domains using RaxML. The PROTGAMMALG model was used with 2000 rapid bootstraps. For both (20A) and (20B), the tree was rooted on the IsrB family. Associations are calculated for each cluster based on non-redundant loci (at 90% sequence identity of the main locus ORF). Ga-Gi refer to the IscB/IsrB main ωRNA profiles in FIG. 13A. HNH domain associations are shown with 3 colors, with cyan indicating the HNH domain has the H, N, and H catalytic residues, magenta indicating the HNH domain has the H, N, and N catalytic residues, and grey indicating that the HNH domain has an H, N, and not H/N catalytic residues. Sizes of REC-like insertion in the representative protein sequence for each cluster are shown as determined by the number of amino acids between the BH and the RuvC-II in the alignment. Total size of the representative protein sequence for each cluster is shown on the second outermost ring. Notable groups are shown as labeled colored arcs on the outermost ring.

FIG. 21A-21D—Complete RuvC/BH/HNH phylogenetic analysis of early Cas9 evolution. (21A) Bayesian phylogenetic analysis of IscB and early Cas9 RuvC/BH/HNH domains using MrBayes with random starting trees. The LG substitution model was used with Gamma rates with 4 categories. 4 independent runs were run with 16 chains per with a delta temperature of 0.025 per chain for a total of 10M generations on a GPU for ˜10 days. 1000 swaps were attempted each generation, and tree samples were collected every 50 generations. The average standard deviation of split frequencies was 0.057890 at the final generation. Associations are calculated for each cluster based on non-redundant loci (at 90% sequence identity of the main locus ORF). Ga-Gi refer to the IscB/IsrB main ωRNA profiles in FIG. 13A. HNH domain associations are shown with 3 colors, with cyan indicating the HNH domain has the H, N, and H catalytic residues, magenta indicating the HNH domain has the H, N, and N catalytic residues, and grey indicating that the HNH domain has an H, N, and not H/N catalytic residues. Sizes of REC-like insertion in the representative protein sequence for each cluster are shown as determined by the number of amino acids between the BH and the RuvC-II in the alignment. Total size of the representative protein sequence for each cluster is shown on the second outermost ring. Notable groups are shown as labeled colored arcs on the outermost ring. (21B) Same phylogenetic tree as (21A) with a focus on early Cas9 evolution. Bayesian posterior probabilities for each branch are shown along with the standard deviation of the posterior across all 4 runs. (21C)-(21D) Phylogenetic analysis of the RuvC/BH/HNH domains of early Cas9s and all IscBs using IQ-Tree 2. Each tree is the best scoring ML tree of 5 independent runs. Bootstrap supports were computed with 5000 ultrafast bootstraps. (21C) Phylogenetic analysis using the LG substitution model with gamma rates (4 categories). (21D) Phylogenetic analysis using the LG substitution model with invariant sites and gamma rates (4 categories).

FIG. 22A-22C—IscB/IsrB ωRNA phylogenetic analysis. (22A) Maximum likelihood phylogenetic tree inference for the DNA alignment of ωRNA from IscB/IsrBs using IQ-Tree 2. This tree was built using the best likelihood scoring tree of 200 independent runs as the starting tree with 5000 ultrafast bootstraps (with hill-climbing nearest neighbor change for each bootstrap tree) under the GTR substitution model, using empirical DNA frequencies from the alignment, ascertainment bias correction, and Gamma rates with 4 categories. (22B) Same phylogenetic tree as (22A) but focused on the early Cas9 evolution with the CRISPR-associated IscB cluster 2089. Support values for each branching are shown above the branches. Not all clusters included in other phylogenetic analyses could not be included in this analysis due to lack of a completely alignable ωRNA. For example, clusters 57212 and 50962 were not included. Clusters 2964, 21041, 57212, and 50962 were inferred as ancestral relative to the CRISPR-associated IscB cluster 2089 for the RuvC/BH/HNH amino acid phylogenetic analyses with RaxML (FIG. 35). (22C) Bayesian phylogenetic analysis of tracrRNA like ωRNAs. TracrRNAs from the early Cas9 clusters Cas9_1261 and Cas9_1665 were joined with their respective DRs and separated by a 4 bp poly-A tetraloop. 23 ωRNAs sharing alignment homology to all structural regions from the two tracrRNAs were identified. The resulting 25 RNAs were then aligned with MAFFT-ginsi and manually curated to reduce gappiness. Bayesian phylogenetic analysis of the resulting alignment was performed using MrBayes with 2 chains at a delta temperature of 0.025 with 8 independent runs for 5M generations. A standard GTR model with gamma rates and 4 categories was used. Trees were sampled every 50 generations. The average standard deviation of split frequencies was 0.005966 at the final generation. Bayesian posterior probabilities for each branching are shown above the branch, along with the average standard deviation across the 8 runs. The analysis suggests that the putative modern IscB ancestor of Cas9 (IscB cluster 2089) has an ωRNA descending from the same lineage of ωRNAs that likely resulted in the DR/tracrRNA (Bayesian posterior probability 89%).

FIG. 23A-23D—Comparison of IsrB, IscB and Cas9 subtype features. (57A) Comparison of protein lengths between IsrB, IscB, IscB (large) and Cas9 subtypes identified in this study. The II-D Cas9 group contains members which are substantially smaller than other Cas9 subtypes, while tnpA-associated II-C encompasses some substantially larger members. (23B) P-values resulting from t-tests of pairwise comparison of length distributions shown in (A). (23C) Comparison of median DR lengths for CRISPR arrays associated with IsrB, IscB, IscB (large), where CRISPR-associated, and Cas9 subtypes. Some mhpA-associated II-C loci contain substantially longer DRs (46-47 bp). (23D) Rate of tnpA association with IsrB, IscB, IscB (large) and Cas9 subtypes. 1/545 (0.2%) of unique IsrB loci, 56/2811 (2.0%) of unique IscB loci, including both IscB and IscB (large), and 115/1918 (6.0%) of unique II-C (TnpA) loci are associated with tnpA.

FIG. 24A-24C (SEQ ID NO: 1605-1607)—Cryo-EM structure of IsrB A) depiction of IsrB locus and Cas9 locus and their respective domain organizations; B) cartoon of IsrB and ωRNA at target DNA; C) cryo-EM reconstruction (left) and ribbon diagram (right) of the domain architecture of example IsrB protein and ωRNA.

FIG. 25A-25C (SEQ ID NO: 1608)—ωRNA Structure A) ωRNA sequence structure including stem loops and adaptor pseudoknot (PK) and nexus PK; B) ribbon diagram of the ωRNA structure; C) cleavage assay for ωRNA full length and with various modifications to stemloops and pseudoknots.

FIG. 26—includes depiction of RNA-guided DNA targeting mechanism of example IsrB system.

FIG. 27—ribbon diagram of example IsrB protein with comparison to IscB as depicted in Schuler et al. Science 2022 and Cas9 from Bravo et al. Nature (2022).

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value. It is to be understood that the value to which the modifier “about” or “approximately” refers is itself also specifically, and preferably, disclosed.

The term “about” as used herein when describing an amino acid sequence length or size or a range or ranges of amino acid sequence lengths or sizes are meant to encompass variations of and from the specified value, such as variations in amino acid length or size of +/−5 amino acids.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

A protein or nucleic acid derived from a species means that the protein or nucleic acid has a sequence identical to an endogenous protein or nucleic acid or a portion thereof in the species. The protein or nucleic acid derived from the species may be directly obtained from an organism of the species (e.g., by isolation), or may be produced, e.g., by recombination production or chemical synthesis.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

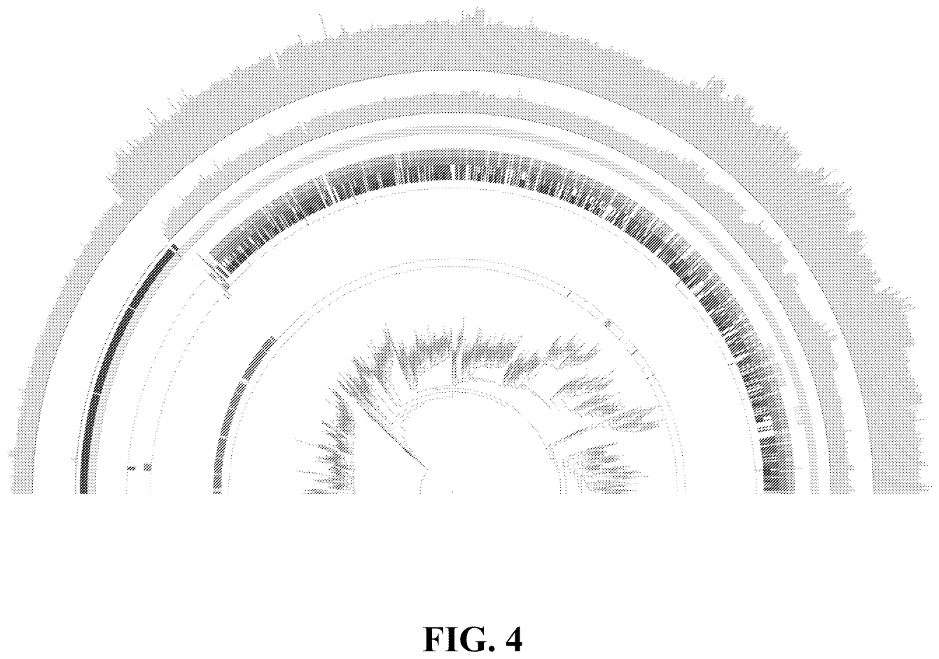

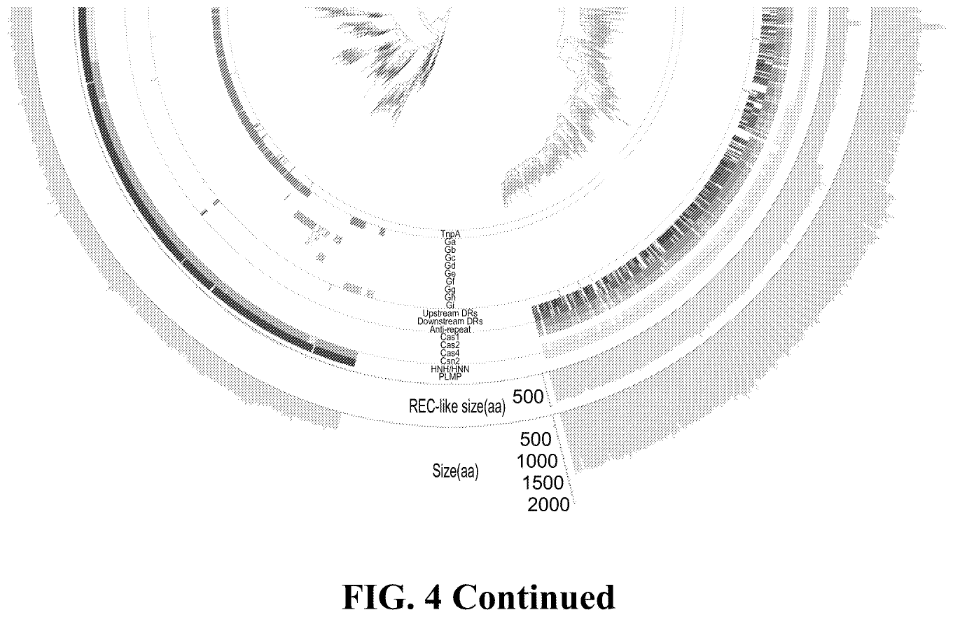

Embodiments disclosed herein provide IsrB systems that function as RNA-guided re-programmable nucleases. An IsrB system comprises a IsrB polypeptide and a nucleic acid component capable of forming a complex with the IsrB polypeptide and directing sequence-specific binding of the complex to a target sequence on the target polynucleotidek. The IsrB system described herein, along with IscB and homologs thereof including IshB, that collectively, along with TnpB systems, may be referred to as OMEGA (Obligate Mobile Element Guided Activity) systems or complexes, or Ω systems or complexes. The nucleic acid component may also be referred to herein as a ωRNA or hRNA. IsrB polypeptides, and homologs thereof, are considerably smaller than other known RNA-guide nucleases, such as Type II and Type V CRISPR-Cas nucleases, and only possess RuvC domain and do not possess a HNH domain. As such, IsrB polypeptides represent a novel class of RNA-guided nucleases that rely on a unique guide RNA structure and may a small size relative to other larger single-effector, RNA-guided nucleases, such as Type II and Type V CRISPR-Cas systems. Due to their smaller size, IsrB may be combined with other functional domains, such as nucleobase deaminases, reverse transcriptases, transposases, ligases, topoisomerases, and serine and threonine recombinases and still be packaged in conventional delivery systems, like certain adenoviruse and lentiviral based viral vectors. Thus, among other improvements, the IsrB system disclosed herein allow more flexible and effective strategies to manipulate and modify target polynucleotides. As shown in FIG. 4, phylogenetic analysis shows IsrB formed a distinct clade from Cas9. IsrB polypeptides are not associated with CRISPR-Cas adaptation genes (cas1, cas2, cas4, and csn2).

In another aspect, embodiments disclosed herein include applications of the IsrB systems, including therapeutics, diagnostics, functional screening, synthetic biology and biomanufacturing. Delivery of the proteins and systems disclosed is also provided, including to a variety of cells and via a variety of delivery systems.

In another aspect, embodiments disclosed herein include applications of the IsrB compositions herein, including diagnostics, therapeutics, and methods of detection. Delivery of the proteins and systems disclosed is also provided, including to a variety of cells and via a variety of particles, vesicles and vectors.

### IsrB Polypeptides

In one embodiment, IsrB polypeptides of the present invention may comprise a split RuvC nuclease domain comprising RuvC-1, Ruv-C II, and Ruv-C III subdomains. In one example embodiment, the RuvC endonuclease domain is split by the insertion of a bridge helix domain. However, unlike Type II CRISPR-Cas proteins, IsrB polypeptides do not contain a Rec domain. In addition, IsrB polypeptides may further comprise a conserved N-terminal domain (also referred to herein as a PLMP domain), which is not present in Cas9 proteins. IsrB proteins may also further comprise a conserved C-terminal domain. IsrBs have a structurally distinct ωRNA.

In one example embodiment, an IsrB polypeptide comprises, moving from the N- to C-terminus, a PLMP domain, a RuvC-I subdomain, a RuvC-II subdomain, a RuvC-III subdomain, and a C terminal domain. In another example embodiment, a bridge helix domain may be inserted between the RuvC-1 and RuvC-II subdomains.

In certain example embodiments, the IsrB polypeptides are between 180 and 600 amino acids in size, between 200 and 590 amino acids in size, between 200 and 780 amino acids in size, between 200 and 570 amino acids in size, between 200 and 560 amino acids in size, between 200 and 550 amino acids in size, between 200 and 540 amino acids in size, between 200 and 530 amino acids in size, between 200 and 520 amino acids in size, between 200 and 510 amino acids in size, between 200 and 500 amino acids in size, between 200 and 490 amino acids in size, between 200 and 480 amino acids in size, between 200 and 470 amino acids in size, between 200 and 460 amino acids in size, between 200 and 450 amino acids in size, between 200 and 440 amino acids in size, between 200 and 430 amino acids in size, between 200 and 420 amino acids in size, between 200 and 410 amino acids in size, between 200 and 400 amino acids in size, between 200 and 390 amino acids in size, between 200 and 380 amino acids in size, between 200 and 370 amino acids in size, between 200 and 360 amino acid, between 200 between 350 amino acids, between 200 and 340 amino acids, between 200 and 330 amino acids, between 200 and 320 amino acids, between 200 and 310 amino acids, between 200 and 300 amino acids, between 200 and 290 amino acids, between 200 and 280 amino acids, between 200 and 270 amino acids, between 200 and 260 amino acids, between 200 and 250 amino acids, between 200 and 240 amino acids, between 200 and 230 amino acids, between 200 and 220 amino acids, between 200 and 210 amino acids, between 200 and 200 amino acids, between 300 and 400 amino acids. Between 300 and 500 amino acids, between 300 and 600 amino acids, between 400 and 500 amino acids, or between 500 and 600 amino acids. In one example embodiment, the polypeptide may range in size from 400-500 amino acids, 400-490 amino acids, 400-480 amino acids, 400-470 amino acids, 400-460 amino acids, 400-450 amino acids, 400-440 amino acids, 400-430 amino acids. Size variation may be dependent, in part, on the particular domain architecture of the IsrB or its homolog.

The IsrB polypeptides may be derived from a naturally occurring protein, a modified naturally occurring protein, functional fragment or truncated version thereof, or a non-naturally occurring protein. In one example embodiments, the IsrBpolypeptide may comprise one or more domains originating from other IsrBpolypeptidenucleases, more particularly originating from different organisms. In an embodiment, the IsrBpolypeptide nucleases may be designed by in silico approaches. Examples of in silico protein design have been described in the art and are therefore known to a skilled person. In particular embodiments, the IsrB polypeptide loci is not associated with a CRISPR array.

The IsrB polypeptides may also encompasses homologs or orthologs of IsrB polypeptides whose sequences are specifically described herein. The terms “ortholog” and “homolog” are well known in the art. By means of further guidance, a “homolog” refers to two genes that share a common ancesteral gene. Homologous proteins may but need not be structurally related or are only partially structurally related. An “ortholog” are two genes that share common ancestral gene but occur in different species. Orthologous proteins may but need not be structurally related or are only partially structurally related. In one embodiment, the homolog or ortholog of a IsrB polypeptide nucleases such as referred to herein has a sequence homology or identity of at least 80%, at least 85%, at least 90%, at least 95% with a IsrB polypeptide nuclease. In further embodiments, the homolog or ortholog of a IsrB polypeptide nuclease has a sequence identity of at least 80%, at least 85%, at least 90%, or at least 95% with a wildtype IsrB polypeptide nuclease, in a particular embodiment the IsrB sequence identified in Table 1 below.

### PLMP Domain

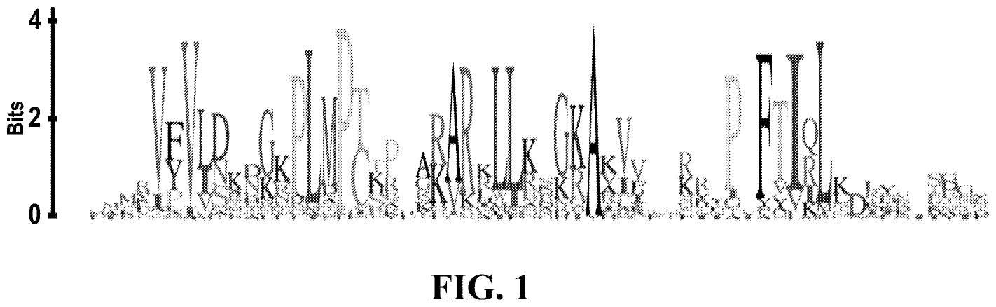

The IsrB polypeptides comprise a conserved N-terminal domain, which is referred to herein as a PLMP domain or an X domain. In embodiments, the N-terminal X domain may have one or more conserved residues and/or motifs as identified in FIG. 1; see also. In one embodiment, the PLMP domain comprises a conserved PLMP (SEQ ID NO:2372) amino acid motif. The PLMP motif can be located at or near the N terminus of the IsrB polypeptide, including, for example at amino acids 17-20 of DthIsrB (SEQ ID NO: 300; SEQ ID NO: 1306), or amino acids corresponding to D. thermocuniculi DSM 16036 IsrB, and at amino acids 12-15 for KraIsrB (SEQ ID NO: 128), or amino acids corresponding to K. racemifer DSM 44963 IsrB.

In some examples, the PLMP domain may be no more than 10, no more than 20, no more than 30, no more than 40, no more than 50, no more than 60, no more than 70, no more than 80, no more than 90, or no more than 100 amino acids in length. For example, the PLMP domain may be no more than 70 amino acids in length, such as comprising 2 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 amino acids in length. An example PLMP domain can be as identified in, e.g. FIG. 58. PLMP domains may be found upstream of the RuvC-I domain and/or Bridge Helix, where present, of an IsrB polypeptide. In one embodiment, the PLMP domain is located within 150, 140, 130, 120, 110, 100, 90, 80, 70, 60, 50, 40, 30, 20 or 10 amino acids upstream of the RuvC-1 domain.

In an aspect, truncation of the N-terminus domain of an IsrB polypeptide, including. more than 4, 5, 6, 7, 8, 9, 10, 11, 12, 13, 14 or 15 amino acids, up to 70 amino acids of the N terminus, i.e. truncation of the PLMP domain, abolishes activity of the IsrB IsrB polypeptide. In an aspect, more than 4 amino acids PLMP domain may reduce or abolish IscB activity. C-terminal domain.

### RuvC Domain

The RuvC domain of the IsrB polypeptide may comprise multiple subdomains, e.g., RuvC-I, RuvC-II and RuvC-III. The subdomains may be separated by interval sequences on the amino acid sequence of the protein.

Examples of RuvC domains include any polypeptides having a structural similarity and/or sequence similarity to a RuvC domain described in the art. For example, the RuvC domain may share a structural similarity and/or sequence similarity to a RuvC of Cas9. In some examples, the RuvC domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with RuvC domains.

In some examples, the RuvC domain comprise RuvC-I polypeptide, RuvC-II polypeptide, and RuvC-III polypeptide. Examples of the RuvC-I domain also include any polypeptides having a structural similarity and/or sequence similarity to a RuvC-I domain described in the art. For example, the RuvC-I domain may share a structural similarity and/or sequence similarity to a RuvC-I of Cas9. In some examples, the RuvC domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with RuvC-I domain. The RuvC-II domain also include any polypeptides a structural similarity and/or sequence similarity to a RuvC-II domain described in the art. For example, the RuvC-II domain may share a structural similarity and/or sequence similarity to a RuvC-II of Cas9. In some examples, the RuvC domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with RuvC-II domains. The RuvC-III domain also include any polypeptides a structural similarity and/or sequence similarity to a RuvC-III domain described in the art. For example, the RuvC-III domains may share a structural similarity and/or sequence similarity to a RuvC-III of Cas9. In some examples, the RuvC domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with RuvC-III domains.

For example, and as described in the art (e.g., Crystal structure of Cas9 in complex with guide RNA and target DNA, Nishimasu et al. Cell, 2014) the RuvC domain of Cas9 consists of a six-stranded mixed β-sheet (β1, β2, β5, β11, β14 and β17) flanked by α-helices (α33, α34 and α39-α45) and two additional two-stranded antiparallel β-sheets (β3/β4 and β15/β16). It has been described that the RuvC domain of Cas9 shares structural similarity with the retroviral integrase superfamily members characterized by an RNase H fold, such as Escherichia coli RuvC (PDB code 1HJR, 14% identity, root-mean-square deviation (rmsd) of 3.6 Å for 126 equivalent Ca atoms) and Thermus thermophilus RuvC (PDB code 4LD0, 12% identity, rmsd of 3.4 Å for 131 equivalent Ca atoms). E. coli RuvC is a 3-layer alpha-beta sandwich containing a 5-stranded beta-sheet sandwiched between 5 alpha-helices. RuvC nucleases have four catalytic residues (e.g., Asp7, Glu70, His143 and Asp146 in T. thermophilus RuvC), and cleave Holliday junctions (or structurally analogous cruciform junctions) through a two-metal mechanism. Asp10 (Ala), Glu762, His983 and Asp986 of the Cas9 RuvC domain are located at positions similar to those of the catalytic residues of T. thermophilus RuvC.

In an aspect, the IsrB comprises an inactive RuvC domain. In one embodiment the IsrB polypeptide comprising an inactive RuvC domain comprises a sequence selected from SEQ ID NO: 1445-1523.

### Bridge Helix

The nucleic-acid guided nuclease comprises a bridge helix (BH) domain. The bridge helix domain refers to a helix and arginine rich polypeptide. The bridge helix domain may be located next to anyone of the amino acid domains in the nucleic-acid guided nuclease. In one embodiment, the bridge helix domain is next to a RuvC domain, e.g., next to RuvC-I, RuvC-II, or RuvC-III subdomain. In one example, the bridge helix domain is between a RuvC-1 and RuvC2 subdomains.

The bridge helix domain may be from 10 to 100, from 20 to 60, from 30 to 50, e.g., 35, 36, 37, 38, 39, 40, 41, 42, 43, 44, 45, 46 or 47, 48, 49, or 50 amino acids in length. Examples of bridge helix includes the polypeptide of amino acids 60-93 of the sequence of S. pyogenes Cas9.

In an embodiment, examples of the BH domain include those in Table 1. Examples of the BH domain also include any polypeptides a structural similarity and/or sequence similarity to a BH domain described in the art. For example, the BH domain may share a structural similarity and/or sequence similarity to a BH domain of Cas9. In some examples, the BH domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with BH domains in Table 1.

### C-Terminal Domain

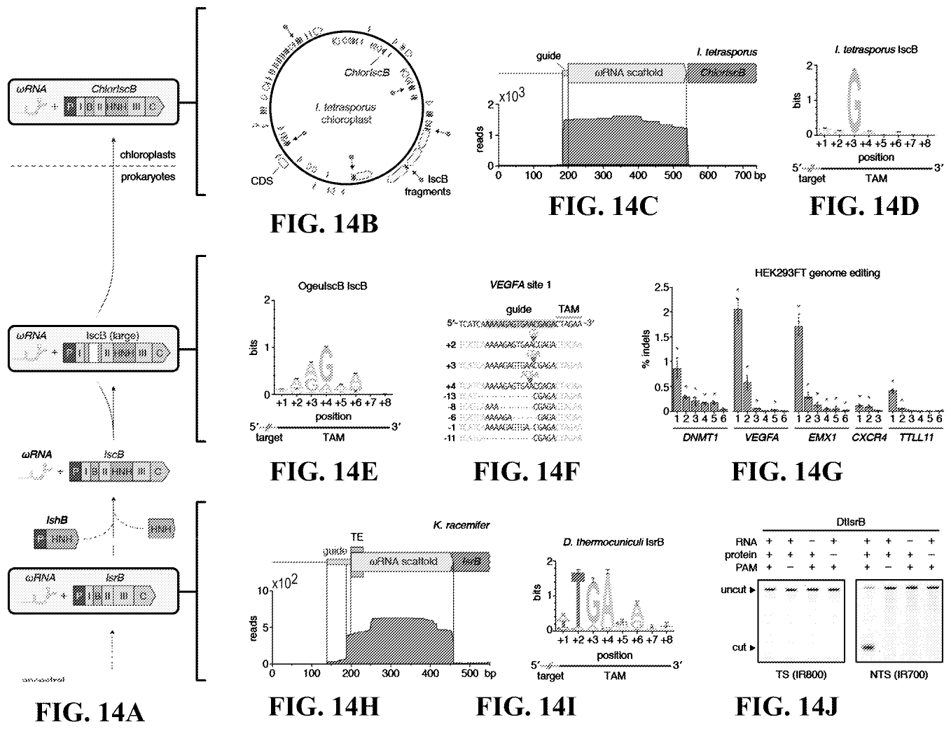

The C-terminal domain (also referred to herein as a Y domain) may comprise one or more conserved residues or motifs as shown in FIG. 14A. The C-terminal domain may be no more than 10, no more than 20, no more than 30, no more than 40, no more than 50, no more than 60, no more than 70, no more than 80, no more than 90, or no more than 100 amino acids in length. For example, the C-terminal domain may be no more than 70 amino acids in length, such as comprising 2 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 amino acids in length.

### Example IsrB Polypeptides

The following table provides example IsrB polypeptides that may be used in the IsrB systems disclosed herein.

TABLE 1
Exemplary IsrB Polypeptides

| SEQ ID NO | Contig Desc |
|---|---|
| 1 | Scytonema hofmannii PCC 7110 genomic scaffold Scaffold1, whole genome shotgun |
| | sequence |
| 2 | Scytonema sp. HK-05 DNA, nearly complete genome |
| 3 | Scytonema sp. HK-05 DNA, nearly complete genome |
| 4 | Ga0315277_10038595 |
| 5 | Moorea producens PAL-8-15-08-1 chromosome, complete genome |
| 6 | Geitlerinema sp. PCC 7105 genomic scaffold Gei7105DRAFT_GPC.5, whole |
| | genome shotgun sequence |
| 7 | Arthrospira platensis str. Paraca isolate UASWS Contig183, whole genome shotgun |
| | sequence |
| 8 | Microcystis aeruginosa 9443 WGS project CAIJ01000000 data, contig |
| | AAI_C_2197_378, whole genome shotgun sequence |
| 9 | Moorea bouillonii PNG strain PNG5-198 Ga0081470_101, whole genome shotgun |
| | sequence |
| 10 | Moorea producens 3L Ga0081465_101, whole genome shotgun sequence |
| 11 | Microcoleus sp. IPPAS B-353 genomic scaffold scaffold1, whole genome shotgun |
| | sequence |
| 12 | Spirulina major PCC 6313 genomic scaffold Contig40, whole genome shotgun |
| | sequence |
| 13 | Spirulina major PCC 6313 genomic scaffold Contig40, whole genome shotgun |
| | sequence |
| 14 | Scytonema sp. HK-05 DNA, nearly complete genome |
| 15 | Scytonema sp. NIES-4073 DNA, nearly complete genome |
| 16 | Candidatus Poribacteria bacterium isolate SB0663_bin_6 |
| | NODE_237_length_23550_cov_6.594254, whole genome shotgun sequence |
| 17 | Candidatus Poribacteria bacterium isolate PCPOR1 Ga0206387_1056, whole |
| | genome shotgun sequence |
| 18 | Scytonema sp. HK-05 DNA, nearly complete genome |
| 19 | Scytonema sp. HK-05 DNA, nearly complete genome |
| 20 | Scytonema sp. NIES-4073 DNA, nearly complete genome |
| 21 | Calothrix sp. NIES-4105 DNA, nearly complete genome |
| 22 | Scytonema sp. NIES-4073 DNA, nearly complete genome |
| 23 | Scytonema sp. HK-05 DNA, nearly complete genome |
| 24 | Scytonema sp. HK-05 DNA, nearly complete genome |
| 25 | Scytonema sp. NIES-4073 DNA, nearly complete genome |
| 26 | metagenome genome assembly, contig: |
| | NODE_3439_length_13191_cov_268.301586, whole genome shotgun sequence |
| 27 | Calothrix sp. NIES-2100 DNA, nearly complete genome |
| 28 | Scytonema sp. NIES-4073 DNA, nearly complete genome |
| 29 | Scytonema sp. HK-05 DNA, nearly complete genome |
| 30 | Symploca sp. SIO2C1 2C1_NODE_99, whole genome shotgun sequence |
| 31 | Scytonema sp. NIES-4073 DNA, nearly complete genome |
| 32 | Scytonema sp. NIES-4073 DNA, nearly complete genome |
| 33 | Scytonema sp. HK-05 DNA, nearly complete genome |
| 34 | Tychonema bourrellyi FEM_GT703 scaffold165_size8795, whole genome shotgun |
| | sequence |
| 35 | Scytonema sp. HK-05 DNA, nearly complete genome |
| 36 | Scytonema sp. HK-05 DNA, nearly complete genome |
| 37 | Scytonema sp. HK-05 DNA, nearly complete genome |
| 38 | Scytonema sp. HK-05 DNA, nearly complete genome |
| 39 | Symploca sp. SIO2B6 2B6_NODE_3, whole genome shotgun sequence |
| 40 | Calothrix sp. NIES-4071 DNA, complete genome |
| 41 | Planktothrix prolifica NIVA-CYA 406 contig00287, whole genome shotgun |
| | sequence |
| 42 | Moorea producens PAL-8-15-08-1 chromosome, complete genome |
| 43 | Calothrix sp. NIES-4105 DNA, nearly complete genome |
| 44 | TPA_asm: Cyanobacteria bacterium UBA11162 contig_8303, whole genome |
| | shotgun sequence |
| 45 | Trichormus variabilis SAG 1403-4b sequence01, whole genome shotgun sequence |
| 46 | Calothrix sp. NIES-4071 DNA, complete genome |
| 47 | Calothrix sp. NIES-4105 DNA, nearly complete genome |
| 48 | Moorea producens PAL-8-15-08-1 chromosome, complete genome |
| 49 | Moorea producens JHB sequence |
| 50 | Spirulina major PCC 6313 Contig40_C6, whole genome shotgun sequence |
| 51 | Calothrix sp. NIES-4071 DNA, complete genome |
| 52 | Moorea producens 3L Ga0081465_101, whole genome shotgun sequence |
| 53 | Moorea producens JHB sequence |
| 54 | Moorea producens PAL strain NAK12DEC93-3La Ga0081465_101, whole genome |
| | shotgun sequence |
| 55 | Calothrix sp. PCC 6303, complete genome |
| 56 | Moorea producens JHB sequence |
| 57 | Moorea sp. SIO3I6 3I6_NODE_94, whole genome shotgun sequence |
| 58 | Moorea producens 3L Ga0081465_101, whole genome shotgun sequence |
| 59 | Tolypothrix bouteillei VB521301 NODE_1_length_9494340_cov_10.278967, whole |
| | genome shotgun sequence |
| 60 | Moorea producens 3L Ga0081465_101, whole genome shotgun sequence |
| 61 | Ardenticatenales bacterium isolate MGR_bin47 SD2897-2912_k127_7054962, |
| | whole genome shotgun sequence |
| 62 | Moorea producens PAL strain NAK12DEC93-3La Ga0081465_101, whole genome |
| | shotgun sequence |
| 63 | Moorea producens PAL strain NAK12DEC93-3La Ga0081465_101, whole genome |
| | shotgun sequence |
| 64 | Calothrix brevissima NIES-22 DNA, nearly complete genome |
| 65 | Moorea producens 3L Ga0081465_101, whole genome shotgun sequence |
| 66 | Moorea producens PAL strain NAK12DEC93-3La Ga0081465_101, whole genome |
| | shotgun sequence |
| 67 | Moorea bouillonii PNG strain PNG5-198 Ga0081470_101, whole genome shotgun |
| | sequence |
| 68 | Moorea bouillonii PNG strain PNG5-198 Ga0081470_101, whole genome shotgun |
| | sequence |
| 69 | Microcystis panniformis FACHB-1757, complete genome |
| 70 | Moorea bouillonii PNG strain PNG5-198 Ga0081470_101, whole genome shotgun |
| | sequence |
| 71 | Moorea producens PAL strain NAK12DEC93-3La Ga0081465_101, whole genome |
| | shotgun sequence |
| 72 | Microcoleus sp. Co-bin12 k141_6726532_length_3994_cov_14.2126, whole genome |
| | shotgun sequence |
| 73 | Microcystis sp. MC19 chromosome, complete genome |
| 74 | Microcystis panniformis FACHB-1757, complete genome |
| 75 | Calothrix parasitica NIES-267 DNA, nearly complete genome |
| 76 | Calothrix parasitica NIES-267 DNA, nearly complete genome |
| 77 | Burkholderiales bacterium isolate ES-bin-76 ES-bin-76-contig-k141_6057916, |
| | whole genome shotgun sequence |
| 78 | Moorea bouillonii PNG strain PNG5-198 Ga0081470_101, whole genome shotgun |
| | sequence |
| 79 | Moorea bouillonii PNG strain PNG5-198 Ga0081470_101, whole genome shotgun |
| | sequence |
| 80 | Nostoc flagelliforme CCNUN1 chromosome, complete genome |
| 81 | Moorea sp. SIO3I6 3I6_NODE_94, whole genome shotgun sequence |
| 82 | Fischerella sp. NIES-4106 DNA, nearly complete genome |
| 83 | Brasilonema sp. UFV-L1 scaffold148_cov13, whole genome shotgun sequence |
| 84 | Moorea sp. SIO2C4 2C4_NODE_889, whole genome shotgun sequence |
| 85 | Arthrospira sp. str. PCC 8005 chromosome, complete genome |
| 86 | Microcoleus sp. PCC 7113, complete genome |
| 87 | Arthrospira sp. TJSD092 chromosome, complete genome |
| 88 | Limnospira sp. BM chromosome |
| 89 | Microcystis panniformis FACHB-1757, complete genome |
| 90 | Microcoleus sp. IPPAS B-353 Scaffolds01.1, whole genome shotgun sequence |
| 91 | Moorea sp. SIO2C4 2C4_NODE_889, whole genome shotgun sequence |
| 92 | Anabaena variabilis NIES-23 plasmid plasmid3 DNA, nearly complete genome |
| 93 | Nodularia sp. NIES-3585 DNA, scaffold: scaffold1, whole genome shotgun sequence |
| 94 | Oscillatoria acuminata PCC 6304, complete genome |
| 95 | Moorea sp. SIO3B2 3B2_NODE_853, whole genome shotgun sequence |
| 96 | Microcoleus sp. PCC 7113, complete genome |
| 97 | Deltaproteobacteria bacterium isolate B7_G9 B7_Guay9_scaffold_45962, whole |
| | genome shotgun sequence |
| 98 | Limnospira fusiformis SAG 85.79 chromosome, complete genome |
| 99 | Arthrospira platensis C1 chromosome, whole genome shotgun sequence |
| 100 | Microcoleus sp. PCC 7113, complete genome |
| 101 | Calothrix desertica PCC 7102 sequence007, whole genome shotgun sequence |
| 102 | Microcoleus sp. PCC 7113, complete genome |
| 103 | Aphanocapsa montana BDHKU210001 |
| | NODE_102_length_99650_cov_131.448837, whole genome shotgun sequence |
| 104 | Oscillatoria acuminata PCC 6304, complete genome |
| 105 | Ga0373628_0092043 |
| 106 | sediment metagenome genome assembly, contig: |
| | NODE_771_length_39337_cov_4.259322, whole genome shotgun sequence |
| 107 | Microcystis viridis NIES-102 DNA, complete genome |
| 108 | Planktothrix sp. FACHB-1365 contig1, whole genome shotgun sequence |
| 109 | Arthrospira platensis NIES-39, *** SEQUENCING IN PROGRESS ***, 19 ordered |
| | pieces |
| 110 | Arthrospira platensis NIES-39, *** SEQUENCING IN PROGRESS ***, 19 ordered |
| | pieces |
| 111 | Microcystis aeruginosa FD4 chromosome, complete genome |
| 112 | Planktothrix prolifica NIVA-CYA 540 genomic scaffold scaffold00001, whole |
| | genome shotgun sequence |
| 113 | Planktothrix prolifica NIVA-CYA 98 genomic scaffold scaffold00001, whole |
| | genome shotgun sequence |
| 114 | Ga0315279_10002905 |
| 115 | Microcystis viridis NIES-102 DNA, complete genome |
| 116 | Ga0315284_10001184 |
| 117 | Chroococcidiopsis sp. PCC 6712 Ga0438089_03, whole genome shotgun sequence |
| 118 | Nostoc sphaeroides CCNUC1 chromosome Gxm1, complete sequence |
| 119 | Scytonema millei VB511283 scaffold_1, whole genome shotgun sequence |
| 120 | Sediment metagenome Contig_107, whole genome shotgun sequence |
| 121 | Leptolyngbyaceae cyanobacterium CCMR0082 Scaffold_1b, whole genome shotgun |
| | sequence |
| 122 | Planktothrix prolifica NIVA-CYA 98 genomic scaffold scaffold00001, whole |
| | genome shotgun sequence |
| 123 | Anabaena sp. CA = ATCC 33047 contig081, whole genome shotgun sequence |
| 124 | Planktothrix prolifica NIVA-CYA 540 genomic scaffold scaffold00001, whole |
| | genome shotgun sequence |
| 125 | Moorea sp. SIO3I8 3I8_NODE_130, whole genome shotgun sequence |
| 126 | Anabaena sp. WA102, complete genome |
| 127 | Planktothrix prolifica NIVA-CYA 406 genomic scaffold scaffold00001, whole |
| | genome shotgun sequence |
| 128 | Ktedonobacter racemifer DSM 44963 strain SOSP1-21 Krac_Contig206, whole |
| | genome shotgun sequence |
| 129 | Arthrospira platensis YZ genome |
| 130 | Planktothrix prolifica NIVA-CYA 98 genomic scaffold scaffold00001, whole |
| | genome shotgun sequence |
| 131 | Pleurocapsa sp. PCC 7319 genomic scaffold Pleur7319scaffold_7, whole genome |
| | shotgun sequence |
| 132 | Arthrospira platensis YZ genome |
| 133 | Microcystis panniformis FACHB-1757, complete genome |
| 134 | Calothrix anomala FACHB-343 contig2, whole genome shotgun sequence |
| 135 | Nodularia sp. NIES-3585 DNA, scaffold: scaffold1, whole genome shotgun sequence |
| 136 | Oscillatoriales cyanobacterium RU_3_3 NODE_1980_length_14615_cov_2.286996, |
| | whole genome shotgun sequence |
| 137 | Spirulina major PCC 6313 genomic scaffold Contig40, whole genome shotgun |
| | sequence |
| 138 | Mastigocladopsis repens PCC 10914 genomic scaffold |
| | Mas10914DRAFT_scaffold1.1, whole genome shotgun sequence |
| 139 | Microcystis aeruginosa NIES-843 DNA, complete genome |
| 140 | Fischerella sp. PCC 9605 FIS9605DRAFT_scaffold12.12_C, whole genome shotgun |
| | sequence |
| 141 | Tolypothrix [Scytonema hofmanni] UTEX 2349 genomic scaffold |
| | Tol9009DRAFT_TPD.8, whole genome shotgun sequence |
| 142 | Spirulina major PCC 6313 genomic scaffold Contig40, whole genome shotgun |
| | sequence |
| 143 | Microcoleus chthonoplastes PCC 7420 scf_1103659003828 genomic scaffold, whole |
| | genome shotgun sequence |
| 144 | Cyanothece sp. PCC 7822, complete genome |
| 145 | Thermanaeromonas toyohensis ToBE genome assembly, chromosome: I |
| 146 | Leptolyngbyaceae cyanobacterium CCMR0082 Scaffold_1b, whole genome shotgun |
| | sequence |
| 147 | Planktothrix prolifica NIVA-CYA 540 genomic scaffold scaffold00001, whole |
| | genome shotgun sequence |
| 148 | Microcystis aeruginosa FD4 chromosome, complete genome |
| 149 | Ga0373625_0039173 |
| 150 | Planktothrix prolifica NIVA-CYA 406 genomic scaffold scaffold00001, whole |
| | genome shotgun sequence |
| 151 | Moorea sp. SIO3B2 3B2_NODE_23, whole genome shotgun sequence |
| 152 | Microcystis viridis NIES-102 DNA, complete genome |
| 153 | Spirulina major PCC 6313 genomic scaffold Contig40, whole genome shotgun |
| | sequence |
| 154 | Microcystis aeruginosa NIES-4264 DNA, contig_41, whole genome shotgun |
| | sequence |
| 155 | Cyanothece sp. PCC 7822, complete genome |
| 156 | Dolichospermum sp. UHCC 0315A chromosome, complete genome |
| 157 | Cyanothece sp. PCC 7822, complete genome |
| 158 | Mastigocladopsis repens PCC 10914 genomic scaffold |
| | Mas10914DRAFT_scaffold1.1, whole genome shotgun sequence |
| 159 | Tolypothrix [Scytonema hofmanni] UTEX 2349 genomic scaffold |
| | Tol9009DRAFT_TPD.8, whole genome shotgun sequence |
| 160 | Dolichospermum sp. UHCC 0315A chromosome, complete genome |
| 161 | Microcystis aeruginosa NIES-843 DNA, complete genome |
| 162 | Chroococcidiopsis sp. PCC 6712 Ga0438089_03, whole genome shotgun sequence |
| 163 | Spirulina major PCC 6313 genomic scaffold Contig40, whole genome shotgun |
| | sequence |
| 164 | Ga0315284_10185211 |
| 165 | Mastigocladopsis repens PCC 10914 genomic scaffold |
| | Mas10914DRAFT_scaffold1.1, whole genome shotgun sequence |
| 166 | Microcystis panniformis FACHB-1757, complete genome |
| 167 | Cyanothece sp. PCC 7822, complete genome |
| 168 | Ga0315273_10142751 |
| 169 | Spirulina major PCC 6313 genomic scaffold Contig40, whole genome shotgun |
| | sequence |
| 170 | Microcystis sp. MC19 chromosome, complete genome |
| 171 | Ktedonobacter racemifer DSM 44963 strain SOSP1-21 Krac_Contig206, whole |
| | genome shotgun sequence |
| 172 | Spirulina major PCC 6313 genomic scaffold Contig40, whole genome shotgun |
| | sequence |
| 173 | Geitlerinema sp. PCC 7105 genomic scaffold Gei7105DRAFT_GPC.5, whole |
| | genome shotgun sequence |
| 174 | Anabaena sp. PCC 7108 genomic scaffold Ana7108scaffold_2, whole genome |
| | shotgun sequence |
| 175 | Anabaena sp. 90 chromosome chANA01, complete sequence |
| 176 | Hapalosiphonaceae cyanobacterium JJU2 Scaffold_61, whole genome shotgun |
| | sequence |
| 177 | Ktedonobacter racemifer DSM 44963 strain SOSP1-21 Krac_Contig205, whole |
| | genome shotgun sequence |
| 178 | Spirulina major PCC 6313 genomic scaffold Contig40, whole genome shotgun |
| | sequence |
| 179 | Firmicutes bacterium R50 isolate R501 genome assembly, chromosome: 1 |
| 180 | Oscillatoriales cyanobacterium isolate PH2015_11S_45_847 |
| | PH2015_11S_scaffold_340, whole genome shotgun sequence |
| 181 | Dolichospermum compactum NIES-806 DNA, nearly complete genome |
| 182 | Dactylococcopsis salina PCC 8305, complete genome |
| 183 | Planktothrix agardhii NIVA-CYA 34 contig00043, whole genome shotgun sequence |
| 184 | Microcystis aeruginosa PCC 7005 Mic7005contig745, whole genome shotgun |
| | sequence |
| 185 | Spirulina subsalsa PCC 9445 genomic scaffold Contig210, whole genome shotgun |
| | sequence |
| 186 | Spirulina subsalsa PCC 9445 genomic scaffold Contig210, whole genome shotgun |
| | sequence |
| 187 | Ga0373626_0090068 |
| 188 | Crinalium epipsammum PCC 9333, complete genome |
| 189 | Subsurface metagenome NODE_351_length_4665_cov_5.720981, whole genome |
| | shotgun sequence |
| 190 | fermentation metagenome genome assembly, contig: |
| | NODE_49_length_109118_cov_31.646049, whole genome shotgun sequence |
| 191 | Dactylococcopsis salina PCC 8305, complete genome |
| 192 | Spirulina major PCC 6313 Contig40_C7, whole genome shotgun sequence |
| 193 | Candidatus Poribacteria bacterium isolate SB0675_bin_22 |
| | NODE_1111_length_21226_cov_11.928865, whole genome shotgun sequence |
| 194 | Candidatus Poribacteria bacterium isolate Plut_88885 |
| | unfiltered_Plut_88885_genomic_contig_528529, whole genome shotgun sequence |
| 195 | Microcystis aeruginosa NIES-2481, complete genome |
| 196 | Geitlerinema sp. PCC 7105 genomic scaffold Gei7105DRAFT_GPC.5, whole |
| | genome shotgun sequence |
| 197 | Microcystis sp. M_QC_C_20170808_M9Col M9Col_31, whole genome shotgun |
| | sequence |
| 198 | Halothece sp. PCC 7418, complete genome |
| 199 | Microcystis aeruginosa NIES-2549, complete genome |
| 200 | Pleurocapsa sp. PCC 7319 Pleur7319scaffold_7_Cont24, whole genome shotgun |
| | sequence |
| 201 | Microcystis aeruginosa NIES-2549, complete genome |
| 202 | Synechococcus sp. PCC 7335 scf_1103496006895 genomic scaffold, whole genome |
| | shotgun sequence |
| 203 | Moorea sp. SIO1G6 1G6_NODE_107, whole genome shotgun sequence |
| 204 | Ktedonobacter racemifer DSM 44963 strain SOSP1-21 Krac_Contig203, whole |
| | genome shotgun sequence |
| 205 | TPA_asm: Microcoleaceae bacterium UBA11344 contig_6690, whole genome |
| | shotgun sequence |
| 206 | Microcystis aeruginosa NIES-2481, complete genome |
| 207 | Planktothrix rubescens strain PCC 7821 genome assembly, contig: |
| | AAI_N _LAN_Contig_1, whole genome shotgun sequence |
| 208 | Microcystis flos-aquae TF09 NODE_1_length_1219774_cov_159.253, whole |
| | genome shotgun sequence |
| 209 | Scytonema sp. HK-05 NIES-2130_Scaffold_43, whole genome shotgun sequence |
| 210 | Scytonema sp. HK-05 NIES-2130_Scaffold_10, whole genome shotgun sequence |
| 211 | Halothece sp. PCC 7418, complete genome |
| 212 | Sulfobacillus thermosulfidooxidans strain CBAR-13 Scaffold_1_CBAR13, whole |
| | genome shotgun sequence |
| 213 | Acidithiobacillus ferrivorans WGS project CCCS000000000 data, strain CF27, |
| | contig ATN_AFERRI_Contig_8, whole genome shotgun sequence |
| 214 | Spirulina major PCC 6313 Contig40_C7, whole genome shotgun sequence |
| 215 | Desulfitobacterium hafniense TCP-A genomic scaffold DeshafDRAFT_Scaffold2.2, |
| | whole genome shotgun sequence |
| 216 | Symploca sp. SIO1A3 1A3_NODE_818, whole genome shotgun sequence |
| 217 | Dactylococcopsis salina PCC 8305, complete genome |
| 218 | Arthrospira platensis NIES-46 DNA, sequence091, whole genome shotgun sequence |
| 219 | Ktedonobacter racemifer DSM 44963 strain SOSP1-21 Krac_Contig203, whole |
| | genome shotgun sequence |
| 220 | uncultured cyanobacterium isolate I3b_bin-335 genome assembly, contig: bin- |
| | 335: 0468/1049, whole genome shotgun sequence |
| 221 | Microcoleus sp. FACHB-45 contig101, whole genome shotgun sequence |
| 222 | Ktedonobacter racemifer DSM 44963 strain SOSP1-21 Krac_Contig203, whole |
| | genome shotgun sequence |
| 223 | Acidithiobacillus ferrivorans isolate PRJEB5721 genome assembly, chromosome: |
| | AFERRI |
| 224 | Microcystis aeruginosa BS13-02 Ga0188020_100022, whole genome shotgun |
| | sequence |
| 225 | Symploca sp. SIO1C4 1C4_NODE_773, whole genome shotgun sequence |
| 226 | Microcystis aeruginosa NaRes975 Scaffold58_1, whole genome shotgun sequence |
| 227 | Calothrix rhizosoleniae SC01 genome assembly, contig: contig69601, whole genome |
| | shotgun sequence |
| 228 | Acidithiobacillus thiooxidans ATCC 19377 chromosome, complete genome |
| 229 | Westiellopsis prolifica IICB1 scaffold_1, whole genome shotgun sequence |
| 230 | Halothece sp. KZN 001 cyano_08_contig_166, whole genome shotgun sequence |
| 231 | Candidatus Poribacteria bacterium isolate PCPOR2b Ga0206366_153, whole |
| | genome shotgun sequence |
| 232 | Westiellopsis prolifica IICB1 scaffold_1, whole genome shotgun sequence |
| 233 | TPA asm: Microcoleaceae bacterium UBA11344 contig_6690, whole genome |
| | shotgun sequence |
| 234 | Hydrococcus sp. RM1_1_31 NODE_5534_length_4416_cov_2.190720, whole |
| | genome shotgun sequence |
| 235 | Microcoleus chthonoplastes PCC 7420 scf_1103659003802 genomic scaffold, whole |
| | genome shotgun sequence |
| 236 | Nostoc sp. ATCC 43529 scaffold6.1, whole genome shotgun sequence |
| 237 | Candidatus Poribacteria bacterium isolate PCPOR1 Ga0206387_1090, whole |
| | genome shotgun sequence |
| 238 | Leptolyngbya boryana IAM M-101 plasmid pLBX DNA, complete genome, strain: |
| | IAM M-101 |
| 239 | Ktedonobacter racemifer DSM 44963 strain SOSP1-21 Krac_Contig203, whole |
| | genome shotgun sequence |
| 240 | Spirulina major PCC 6313 Contig40_C7, whole genome shotgun sequence |
| 241 | Scytonema sp. HK-05 NIES-2130_Scaffold_2, whole genome shotgun sequence |
| 242 | Moorea sp. SIO3A5 3A5_NODE_46, whole genome shotgun sequence |
| 243 | Spirulina major PCC 6313 Contig40_C7, whole genome shotgun sequence |
| 244 | Spirulina major PCC 6313 Contig40_C7, whole genome shotgun sequence |
| 245 | Anabaena cylindrica FACHB-170 contig44, whole genome shotgun sequence |
| 246 | Ktedonobacter racemifer DSM 44963 strain SOSP1-21 Krac_Contig205, whole |
| | genome shotgun sequence |
| 247 | Ga0315280_10000490 |
| 248 | Calothrix desertica PCC 7102 Cal7102DRAFT_CDB.6, whole genome shotgun |
| | sequence |
| 249 | Microcystis aeruginosa KW Contig4, whole genome shotgun sequence |
| 250 | Sulfobacillus thermosulfidooxidans strain CBAR-13 Scaffold_1_CBAR13, whole |
| | genome shotgun sequence |
| 251 | Thermanaeromonas toyohensis ToBE genome assembly, chromosome: I |
| 252 | Microcystis aeruginosa KW Contig4, whole genome shotgun sequence |
| 253 | Oil field metagenome NODE_489_length_3028_cov_3.81188_ID_977, whole |
| | genome shotgun sequence |
| 254 | Cyanothece sp. BG0011 unitig_31, whole genome shotgun sequence |
| 255 | Crocosphaera watsonii WH 0005 WGS project CAQL00000000 data, contig 01056, |
| | whole genome shotgun sequence |
| 256 | Chlorogloeopsis fritschii PCC 6912 sequence18, whole genome shotgun sequence |
| 257 | Cyanobacteria bacterium SW_7_48_12 sw_7_scaffold_1695, whole genome shotgun |
| | sequence |
| 258 | Spirulina major PCC 6313 Contig40_C7, whole genome shotgun sequence |
| 259 | Planktothrix prolifica NIVA-CYA 540 contig00111, whole genome shotgun |
| | sequence |
| 260 | Microcystis aeruginosa KW Contig4, whole genome shotgun sequence |
| 261 | Cyanobacteria bacterium J083 k99_1471286, whole genome shotgun sequence |
| 262 | Coleofasciculus chthonoplastes PCC 7420 ctg_1103659003575, whole genome |
| | shotgun sequence |
| 263 | Ga0315284_10003385 |
| 264 | Moorea sp. SIO4G2 4G2_NODE_2420, whole genome shotgun sequence |
| 265 | Microcystis aeruginosa NIES-2519 DNA, contig_1, whole genome shotgun sequence |
| 266 | Pelotomaculum thermopropionicum SI DNA, complete genome |
| 267 | Hydrocoleum sp. CS-953, whole genome shotgun sequence |
| 268 | Anabaena sp. 4-3 contig107, whole genome shotgun sequence |
| 269 | Caldicellulosiruptor hydrothermalis 108, complete genome |
| 270 | Candidatus Poribacteria bacterium bin44 |
| | NODE_2286_length_30008_cov_19.1247_ID_4571, whole genome shotgun |
| | sequence |
| 271 | Nostoc sp. ATCC 53789 plasmid pNsp_b, complete sequence |
| 272 | Ga0315273_10074057 |
| 273 | sediment metagenome genome assembly, contig: |
| | NODE_2_length_64137_cov 4.613995, whole genome shotgun sequence |
| 274 | Microcystis panniformis Mp_MB_F_20051200_S6 S6_49, whole genome shotgun |
| | sequence |
| 275 | Microcystis aeruginosa 9717 WGS project CAII01000000 data, contig |
| | AAI_B_2196_677, whole genome shotgun sequence |
| 276 | Mastigocladus laminosus UU774 scaffold_2, whole genome shotgun sequence |
| 277 | Symploca sp. SIO2B6 2B6_NODE_1, whole genome shotgun sequence |
| 278 | Brasilonema bromeliae SPC951 NODE_2093_length_16008_cov_5.537140, whole |
| | genome shotgun sequence |
| 279 | Microcystis aeruginosa KW Contig5, whole genome shotgun sequence |
| 280 | Planktothrix mougeotii NIVA-CYA 405 contig00192, whole genome shotgun |
| | sequence |
| 281 | Calothrix rhizosoleniae SC01 genome assembly, contig: contig69601, whole genome |
| | shotgun sequence |
| 282 | Methanoculleus sp. MAB1 isolate Methanoculleus sp MAB1 genome assembly, |
| | chromosome: chrI |
| 283 | Ga0315284_10000757 |
| 284 | Synechococcus sp. PCC 7335 ctg_1103496006870, whole genome shotgun sequence |
| 285 | Westiellopsis prolifica IICB1 scaffold_2, whole genome shotgun sequence |
| 286 | Planktothrix mougeotii NIVA-CYA 405 genomic scaffold scaffold00001, whole |
| | genome shotgun sequence |
| 287 | Calothrix desertica PCC 7102 Cal7102DRAFT_CDB.6, whole genome shotgun |
| | sequence |
| 288 | Anabaena sp. AL93 isolate WA93 214, whole genome shotgun sequence |
| 289 | Aphanizomenon flos-aquae NIES-81 genomic scaffold scaffold00002, whole |
| | genome shotgun sequence |
| 290 | Symploca sp. SIO2B6 2B6_NODE_19, whole genome shotgun sequence |
| 291 | cyanobacterium TDX16 genomic scaffold Scaffold113, whole genome shotgun |
| | sequence |
| 292 | Spirulina major PCC 6313 Contig40_C5, whole genome shotgun sequence |
| 293 | Ga0315280_10053983 |
| 294 | Leptolyngbya valderiana BDU 20041 contig00137, whole genome shotgun sequence |
| 295 | Moorea sp. SIO4A5 4A5_NODE_3, whole genome shotgun sequence |
| 296 | Microcystis aeruginosa KW Contig4, whole genome shotgun sequence |
| 297 | Westiellopsis prolifica IICB1 scaffold_2, whole genome shotgun sequence |
| 298 | Candidatus Poribacteria bacterium isolate PCPOR2 Ga0207196_157, whole genome |
| | shotgun sequence |
| 299 | Oscillatoriales cyanobacterium isolate PH2015_05S_45 1152 |
| | PH2015_05S_scaffold_123, whole genome shotgun sequence |
| 300 | Desulfovirgula thermocuniculi DSM 16036 G454DRAFT_scaffold00006.6_C, whole |
| | genome shotgun sequence |
| 301 | Phormidesmis priestleyi isolate ULC027bin1 scaffold62_size17395, whole genome |
| | shotgun sequence |
| 302 | Ga0315273_10097322 |
| 303 | Microcystis aeruginosa W11-06 Ga0188003_10150, whole genome shotgun |
| | sequence |
| 304 | Microcystis aeruginosa BLCCF108 NODE_334_length_13058_cov_116.817888, |
| | whole genome shotgun sequence |
| 305 | Ga0315284_10006947 |
| 306 | Ga0373630_0001176 |
| 307 | Acaryochloris sp. CCMEE 5410 contig00452, whole genome shotgun sequence |
| 308 | Ga0315294_10064742 |
| 309 | Ga0315280_10007244 |
| 310 | Crocosphaera watsonii WH 8502 WGS project CAQK00000000 data, contig 00305, |
| | whole genome shotgun sequence |
| 311 | Ga0373629_0115667 |
| 312 | Ga0315281_10085784 |
| 313 | Ga0315284_10138768 |
| 314 | Ga0315280_10000925 |
| 315 | TPA_asm: Synechococcus sp. isolate SpSt-164 Ga0101943_1003474, whole genome |
| | shotgun sequence |
| 316 | TPA_asm: Synechococcus sp. isolate SpSt-164 Ga0101943_1001739, whole genome |
| | shotgun sequence |
| 317 | Microcystis aeruginosa NIES-4264 DNA, contig_35, whole genome shotgun |
| | sequence |
| 318 | Candidatus Poribacteria bacterium isolate PCPOR4 Ga0207198_1109, whole |
| | genome shotgun sequence |
| 319 | Ga0315298_1012135 |
| 320 | Chlorogloeopsis fritschii PCC 6912 contig00060, whole genome shotgun sequence |
| 321 | Ga0373626_0016898 |
| 322 | Tolypothrix bouteillei VB521301 scaffold_0, whole genome shotgun sequence |
| 323 | Ardenticatenales bacterium isolate MGR_bin47 SD2897-2912_k127_3100996, |
| | whole genome shotgun sequence |
| 324 | Symploca sp. SIO1C4 1C4_NODE_223, whole genome shotgun sequence |
| 325 | Candidatus Poribacteria bacterium isolate SB0669_bin_10 |
| | NODE_301_length_23714_cov_8.675726, whole genome shotgun sequence |
| 326 | Candidatus Desulfofervidus auxilii strain HS1 genome |
| 327 | Candidatus Poribacteria bacterium isolate SB0669_bin_10 |
| | NODE_388_length_21048_cov_8.115277, whole genome shotgun sequence |
| 328 | Planktothrix rubescens strain 7821 genome assembly, scaffold: |
| | AAI_N_PLAN_scaffold3, whole genome shotgun sequence |
| 329 | Microcystis aeruginosa KW Contig4, whole genome shotgun sequence |
| 330 | Nostoc sp. ATCC 53789 scaffold95.1, whole genome shotgun sequence |
| 331 | TPA_asm: Richelia sp. UBA3957 UBA3957_contig_1031, whole genome shotgun |
| | sequence |
| 332 | Candidatus Poribacteria bacterium isolate SB0662_bin_35 |
| | NODE_1411_length_31134_cov_34.441938, whole genome shotgun sequence |
| 333 | Arthrospira sp. TJSD091 Contig142, whole genome shotgun sequence |
| 334 | Microcystis wesenbergii LE013-01 Ga0066240_1118, whole genome shotgun |
| | sequence |
| 335 | Microcystis aeruginosa 9717 WGS project CAII01000000 data, contig |
| | AAI_B_2196_437, whole genome shotgun sequence |
| 336 | Microcystis flos-aquae Mf_QC_C_20070823_S20T S20T_177, whole genome |
| | shotgun sequence |
| 337 | TPA_asm: Methanoculleus sp. UBA303 UBA303_contig_137, whole genome |
| | shotgun sequence |
| 338 | metagenome genome assembly, contig: NODE_759_length_35023_cov_5.454444, |
| | whole genome shotgun sequence |
| 339 | Cyanobacteria bacterium isolate GSL.Bin21 |
| | NODE_5139_length_10309_cov_0.604633, whole genome shotgun sequence |
| 340 | Ga0315277_10000379 |
| 341 | Microcystis aeruginosa KW Contig4, whole genome shotgun sequence |
| 342 | Microcystis aeruginosa 9701 WGS project CAIQ01000000 data, contig |
| | AAI_K_2204_21, whole genome shotgun sequence |
| 343 | Ga0373630_0073123 |
| 344 | Moorea sp. SIO3A5 3A5_NODE_13, whole genome shotgun sequence |
| 345 | Thermincola ferriacetica strain Z-0001 Tfer_ctg04, whole genome shotgun sequence |
| 346 | Microcystis aeruginosa NIES-1211 DNA, contig 6, whole genome shotgun sequence |
| 347 | Candidatus Poribacteria bacterium isolate SB0663_bin_6 |
| | NODE_466_length_15371_cov_6.406242, whole genome shotgun sequence |
| 348 | Anabaena sp. UHCC 0253 XSP2A_scaffold00001_cov, whole genome shotgun |
| | sequence |
| 349 | Ga0315294_10040325 |
| 350 | Planktothrix rubescens strain PCC 7821 genome assembly, contig: |
| | AAI_N_PLAN_Contig_6, whole genome shotgun sequence |
| 351 | Microcystis aeruginosa PCC 9443 genomic scaffold, AAI_C_2197_scaffold67, |
| | whole genome shotgun sequence |
| 352 | Microcystis aeruginosa TA09 TA09_23, whole genome shotgun sequence |
| 353 | Ga0373630_0000642 |
| 354 | Sulfobacillus sp. DSM 109850 NODE_109_length_10971_cov_117.928952, whole |
| | genome shotgun sequence |
| 355 | Ga0315280_10001324 |
| 356 | Microcystis aeruginosa DA14 NODE_13_length_619060_cov_421.444, whole |
| | genome shotgun sequence |
| 357 | Anabaena variabilis FACHB-171 contig53, whole genome shotgun sequence |
| 358 | Microcoleus chthonoplastes PCC 7420 scf_1103659003820 genomic scaffold, whole |
| | genome shotgun sequence |
| 359 | Microcoleus sp. T1-bin1 k141_138926_length_3681_cov_8.0000, whole genome |
| | shotgun sequence |
| 360 | Candidatus Poribacteria bacterium isolate Plut_88864 |
| | filtered_Plut_88864_genomic_contig_16625, whole genome shotgun sequence |
| 361 | coral metagenome genome assembly, contig: |
| | NODE_156_length_19320_cov_12.549810, whole genome shotgun sequence |
| 362 | Planktothrix mougeotii NIVA-CYA 405 genomic scaffold scaffold00002, whole |
| | genome shotgun sequence |
| 363 | Methanosarcinales archaeon isolate B39_G2 B39_Guay2_scaffold_00908, whole |
| | genome shotgun sequence |
| 364 | Microcystis sp. T1-4 WGS project CAIP01000000 data, contig AAI_I_2203_23, |
| | whole genome shotgun sequence |
| 365 | TPA_asm: Cyanobacteria bacterium UBA11162 contig_7374, whole genome |
| | shotgun sequence |
| 366 | Microcoleus chthonoplastes PCC 7420 scf_1103659003791 genomic scaffold, whole |
| | genome shotgun sequence |
| 367 | Microcoleus sp. SU_5_6 NODE_658_length_15713_cov_3.039843, whole genome |
| | shotgun sequence |
| 368 | Anabaena azotica FACHB-119 contig15, whole genome shotgun sequence |
| 369 | Crocosphaera sp. isolate DT_26 DT-Crocosphaera-1_scaffold_67, whole genome |
| | shotgun sequence |
| 370 | TPA_asm: Cyanobacteria bacterium UBA9273 contig_1024, whole genome shotgun |
| | sequence |
| 371 | TPA_asm: Deltaproteobacteria bacterium UBA4796 UBA4796_contig_73815, |
| | whole genome shotgun sequence |
| 372 | Ga0315284_10078857 |
| 373 | Ga0315284_10003686 |
| 374 | Ga0315284_10024242 |
| 375 | Anabaena cylindrica FACHB-318 contig46, whole genome shotgun sequence |
| 376 | Moorea sp. SIO4G3 4G3_NODE_99, whole genome shotgun sequence |
| 377 | Desulfitobacterium hafniense TCP-A DeshafDRAFT_Scaffold2.2_C7, whole |
| | genome shotgun sequence |
| 378 | Cyanobacteria bacterium QS_7_48_42 qs_7_scaffold_862, whole genome shotgun |
| | sequence |
| 379 | Cyanobacteria bacterium isolate GSL.Bin1 NODE_5738_length_9529_cov_5.21131, |
| | whole genome shotgun sequence |
| 380 | Cyanobacteria bacterium CRU_2_1 NODE_125_length_46571_cov_4.74772, whole |
| | genome shotgun sequence |
| 381 | uncultured cyanobacterium isolate A5_bin-0177 genome assembly, contig: bin- |
| | 0177: 257/358, whole genome shotgun sequence |
| 382 | Moorea sp. SIO3G5 3G5_NODE_1005, whole genome shotgun sequence |
| 383 | wastewater metagenome genome assembly, contig: |
| | NODE_9_length_147903_cov_62.363265, whole genome shotgun sequence |
| 384 | Moorea sp. SIOASIH ASIH_NODE_1, whole genome shotgun sequence |
| 385 | Moorea sp. SIOASIH ASIH_NODE_2, whole genome shotgun sequence |
| 386 | Ga0315279_10000169 |
| 387 | Richelia sp. RM2_1_2 NODE_77_length_54106_cov_31.666926, whole genome |
| | shotgun sequence |
| 388 | Microcystis wesenbergii TW10 TW10_41, whole genome shotgun sequence |
| 389 | wastewater metagenome genome assembly MBR_assembly, scaffold 60651, whole |
| | genome shotgun sequence |
| 390 | TPA_asm: Cyanobacteria bacterium UBA11371 contig_504, whole genome shotgun |
| | sequence |
| 391 | Prochlorothrix hollandica PCC 9006 = CALU 1027 contig_1, whole genome shotgun |
| | sequence |
| 392 | Leptolyngbya boryana PCC 6306 LepboDRAFT_LPC.2_C8, whole genome shotgun |
| | sequence |
| 393 | Ga0373628_0001310 |
| 394 | Moorea sp. SIO318 318_NODE_22, whole genome shotgun sequence |
| 395 | Candidatus Poribacteria bacterium WGA-4E POR4E_contig00010standard_C, whole |
| | genome shotgun sequence |
| 396 | Geitlerinema sp. FC II Abyss71_541 len: 34374, whole genome shotgun sequence |
| 397 | Mastigocladus laminosus UU774 strain 74 scaffold_40, whole genome shotgun |
| | sequence |
| 398 | Moorea sp. SIO3I7 3I7_NODE_59, whole genome shotgun sequence |
| 399 | Microcystis aeruginosa NIES-44 DNA, contig: contig2_19, strain: NIES-44, whole |
| | genome shotgun sequence |
| 400 | Moorea sp. SIO4G3 4G3_NODE_14, whole genome shotgun sequence |
| 401 | Ga0373627_0019696 |
| 402 | Candidatus Poribacteria bacterium isolate SB0678_bin_11 |
| | NODE_3814_length_6294_cov_7.163808, whole genome shotgun sequence |
| 403 | Scytonema sp. HK-05 NIES-2130_Scaffold_97, whole genome shotgun sequence |
| 404 | Ga0315294_10131761 |
| 405 | Candidatus Poribacteria bacterium isolate PCPOR2b Ga0206366_152, whole |
| | genome shotgun sequence |
| 406 | Ga0315273_10003313 |
| 407 | Candidatus Poribacteria bacterium isolate PCPOR2 Ga0207196_147, whole genome |
| | shotgun sequence |
| 408 | Microcystis sp. 0824 DNA, scaffold: scaffold71, strain: 0824, whole genome shotgun |
| | sequence |
| 409 | Microcystis aeruginosa 9808 WGS project CAIN01000000 data, contig |
| | AAI_G_2201_209, whole genome shotgun sequence |
| 410 | TPA_asm: Cyanobacteria bacterium UBA11148 contig_205, whole genome shotgun |
| | sequence |
| 411 | Moorea sp. SIOASIH ASIH_NODE_5, whole genome shotgun sequence |
| 412 | Microcystis sp. 0824 DNA, scaffold: scaffold71, strain: 0824, whole genome shotgun |
| | sequence |
| 413 | Planktothrix agardhii NIVA-CYA 56/3 genomic scaffold scaffold00001, whole |
| | genome shotgun sequence |
| 414 | Nostoc sp. PCC 7120 = FACHB-418 strain PCC 7120 sequence036, whole genome |
| | shotgun sequence |
| 415 | Anabaena sp. UHCC 0253 XSP2A_scaffold00001_cov, whole genome shotgun |
| | sequence |
| 416 | Oscillatoriales cyanobacterium isolate PH2015_08U_46_180 |
| | PH2015_08U_scaffold_98, whole genome shotgun sequence |
| 417 | Candidatus Poribacteria bacterium isolate PCPOR4 Ga0207198_1202, whole |
| | genome shotgun sequence |
| 418 | Calothrix sp. PCC 7103 genomic scaffold Cal7103DRAFT_CPM.1, whole genome |
| | shotgun sequence |
| 419 | Geitlerinema sp. PCC 9228 Ga0115370_1196, whole genome shotgun sequence |
| 420 | Oscillatoriales cyanobacterium isolate PH2015_01U_44_212 |
| | PH2015_01U_scaffold_745, whole genome shotgun sequence |
| 421 | Candidatus Poribacteria bacterium isolate PCPOR4 Ga0207198_1210, whole |
| | genome shotgun sequence |
| 422 | Scytonema tolypothrichoides VB-61278 scaffold_000, whole genome shotgun |
| | sequence |
| 423 | wastewater metagenome genome assembly, contig: |
| | NODE_80_length_54656_cov_7.142946, whole genome shotgun sequence |
| 424 | Prochlorothrix hollandica PCC 9006 = CALU 1027 contig_8, whole genome shotgun |
| | sequence |
| 425 | Ga0315284_10000005 |
| 426 | Mastigocoleus testarum BC008 YY1DRAFT_scaffold_65.66_C, whole genome |
| | shotgun sequence |
| 427 | Oscillatoriales cyanobacterium isolate PH2015_06S_45_83 |
| | PH2015_06S_scaffold_1397, whole genome shotgun sequence |
| 428 | wastewater metagenome genome assembly MBR_assembly, scaffold 53832, whole |
| | genome shotgun sequence |
| 429 | Spirulina subsalsa PCC 9445 Contig203_C, whole genome shotgun sequence |
| 430 | sediment metagenome genome assembly, contig: |
| | NODE_6181_length_5365_cov_4.195726, whole genome shotgun sequence |
| 431 | Microcystis phage Ma-LMM01 DNA, complete genome |
| 432 | Ga0373625_0000082 |
| 433 | Richelia sp. RM1_1_1 NODE_158_length_58252_cov_56.257738, whole genome |
| | shotgun sequence |
| 434 | Moorea sp. SIO3G5 3G5_NODE_511, whole genome shotgun sequence |
| 435 | TPA_asm: Synechococcus sp. isolate SpSt-164 Ga0101943_1003474, whole genome |
| | shotgun sequence |
| 436 | Burkholderiales bacterium isolate ES-bin-76 ES-bin-76-contig-k141_11549369, |
| | whole genome shotgun sequence |
| 437 | Limnoraphis robusta CS-951 contig188, whole genome shotgun sequence |
| 438 | Ga0373625_0162898 |
| 439 | Ga0307928_10003825 |
| 440 | Chloroflexi bacterium isolate B68_G16 B68_Guay16_scaffold_01376, whole |
| | genome shotgun sequence |
| 441 | Aliterella atlantica CENA595 contig_243, whole genome shotgun sequence |
| 442 | Spirulina major PCC 6313 Contig40_C3, whole genome shotgun sequence |
| 443 | Prochlorothrix hollandica PCC 9006 genomic scaffold Pro9006DRAFT_Contig5.9, |
| | whole genome shotgun sequence |
| 444 | Moorea sp. SIO3B2 3B2_NODE_16, whole genome shotgun sequence |
| 445 | Ga0315280_10050637 |
| 446 | Ga0373630_0000013 |
| 447 | Planktothrix rubescens strain 7821 genome assembly, scaffold: |
| | AAI_N_PLAN scaffold1, whole genome shotgun sequence |
| 448 | uncultured cyanobacterium isolate B-1_bin-000 genome assembly, contig: bin- |
| | 000: 459/625, whole genome shotgun sequence |
| 449 | Ga0315280_10061026 |
| 450 | Ga0315298_1008157 |
| 451 | Ga0315294_10133405 |
| 452 | Ga0373630_0072462 |
| 453 | Planktothrix mougeotii NIVA-CYA 405 genomic scaffold scaffold00005, whole |
| | genome shotgun sequence |
| 454 | Crocosphaera watsonii WH0402 WGS project CAQN00000000 data, contig 01993, |
| | whole genome shotgun sequence |
| 455 | Scytonema sp. HK-05 NIES-2130_Scaffold_20, whole genome shotgun sequence |
| 456 | Ga0315285_10055136 |
| 457 | Microcystis aeruginosa NIES-1211 DNA, contig 6, whole genome shotgun sequence |
| 458 | Planktothrix rubescens strain 7821 genome assembly, scaffold: |
| | AAI_N_PLAN scaffold4, whole genome shotgun sequence |
| 459 | Moorea sp. SIOASIH ASIH_NODE_5, whole genome shotgun sequence |
| 460 | Aphanothece sacrum FPU1 DNA, contig: ASFPU1_contig_001, whole genome |
| | shotgun sequence |
| 461 | Microcoleus chthonoplastes PCC 7420 scf_1103659003831 genomic scaffold, whole |
| | genome shotgun sequence |
| 462 | Scytonema hofmannii PCC 7110 Scaffold1_37, whole genome shotgun sequence |
| 463 | Planktothrix agardhii NIVA-CYA 34 contig00043, whole genome shotgun sequence |
| 464 | Oscillatoriales cyanobacterium isolate PH2015_02U_45_393 |
| | PH2015_02U_scaffold_32, whole genome shotgun sequence |
| 465 | TPA_asm: Cyanobacteria bacterium UBA8553 contig_175, whole genome shotgun |
| | sequence |
| 466 | Scytonema sp. HK-05 NIES-2130_Scaffold_125, whole genome shotgun sequence |
| 467 | Microcystis aeruginosa NIES-2519 DNA, contig_1, whole genome shotgun sequence |
| 468 | Scytonema tolypothrichoides VB-61278 scaffold_0, whole genome shotgun |
| | sequence |
| 469 | Microcystis aeruginosa KW Contig5, whole genome shotgun sequence |
| 470 | Ga0315284_10013381 |
| 471 | Geitlerinema sp. PCC 7105 Gei7105DRAFT_GPC.4_C33, whole genome shotgun |
| | sequence |
| 472 | Microcystis aeruginosa NIES-2520 DNA, contig_62, whole genome shotgun |
| | sequence |
| 473 | Microcystis aeruginosa 11-30S32 DNA, 11-30S32contig365, whole genome shotgun |
| | sequence |
| 474 | Oscillatoriales cyanobacterium isolate PH2015_08D_46_1646 |
| | PH2015_08D_scaffold_3729, whole genome shotgun sequence |
| 475 | Scytonema sp. RU_4_4 NODE_633_length_27381_cov_17.449402, whole genome |
| | shotgun sequence |
| 476 | Moorea sp. SIO3E8 3E8_NODE_56, whole genome shotgun sequence |
| 477 | Scytonema sp. UIC 10036 c00130_NODE_13 . . . , whole genome shotgun sequence |
| 478 | metagenomes genome assembly, contig: NODE_1186_length_14052_cov_2.372723, |
| | whole genome shotgun sequence |
| 479 | Mastigocladus laminosus UU774 scaffold_14, whole genome shotgun sequence |
| 480 | Ga0373627_0087747 |
| 481 | uncultured Microcoleus sp. isolate AVDCRST_MAG84 genome assembly, contig: |
| | NODE2305, whole genome shotgun sequence |
| 482 | sediment metagenome genome assembly, contig: |
| | NODE_105_length_5270_cov_4.762416, whole genome shotgun sequence |
| 483 | Ga0315280_10033276 |
| 484 | Leptolyngbya sp. FACHB-402 contig17, whole genome shotgun sequence |
| 485 | Ga0307928_10002244 |
| 486 | Prochlorothrix hollandica PCC 9006 genomic scaffold Pro9006DRAFT_Contig10.1, |
| | whole genome shotgun sequence |
| 487 | Candidatus Poribacteria bacterium bin44 |
| | NODE_2499_length_27701_cov_18.0525_ID_4997, whole genome shotgun |
| | sequence |
| 488 | Microcoleus chthonoplastes PCC 7420 scf_1103659003820 genomic scaffold, whole |
| | genome shotgun sequence |
| 489 | Scytonema sp. HK-05 NIES-2130_Scaffold 72, whole genome shotgun sequence |
| 490 | freshwater metagenome genome assembly, contig: 3777, whole genome shotgun |
| | sequence |
| 491 | Tolypothrix campylonemoides VB511288 scaffold_002, whole genome shotgun |
| | sequence |
| 492 | Candidatus Poribacteria bacterium isolate PCPOR4 Ga0207198_1084, whole |
| | genome shotgun sequence |
| 493 | Moorea sp. SIO4G3 4G3_NODE_88, whole genome shotgun sequence |
| 494 | Anabaena sp. 4-3 contig107, whole genome shotgun sequence |
| 495 | Moorea sp. SIO3F7 3F7_NODE_23, whole genome shotgun sequence |
| 496 | Moorea producens 3L ctg45835, whole genome shotgun sequence |
| 497 | freshwater metagenome genome assembly, contig: 3777, whole genome shotgun |
| | sequence |
| 498 | Spirulina sp. isolate S17.Bin059 Scaffold_8, whole genome shotgun sequence |
| 499 | Planktothrix agardhii NIVA-CYA 56/3 genomic scaffold scaffold00007, whole |
| | genome shotgun sequence |
| 500 | Ga0315284_10121961 |
| 501 | Moorea sp. SIO3A5 3A5_NODE_11, whole genome shotgun sequence |
| 502 | Candidatus Poribacteria bacterium isolate Plut_88888 |
| | unfiltered_Plut_88888_genomic_contig_378005, whole genome shotgun sequence |
| 503 | Okeania sp. SIO2B3 2B3_NODE_8, whole genome shotgun sequence |
| 504 | Microcoleus chthonoplastes PCC 7420 scf_1103659003791 genomic scaffold, whole |
| | genome shotgun sequence |
| 505 | Candidatus Poribacteria bacterium isolate PCPOR1 Ga0206387_1011, whole |
| | genome shotgun sequence |
| 506 | Scytonema sp. HK-05 NIES-2130_Scaffold_6, whole genome shotgun sequence |
| 507 | Chlorogloea sp. CCALA 695 NODE_68_length_40204_cov_6.81018_ID_66494, |
| | whole genome shotgun sequence |
| 508 | Microcystis panniformis Mp_GB_SS_20050300_S99 S99_68, whole genome |
| | shotgun sequence |
| 509 | Leptolyngbya sp. SIO1E4 1E4_NODE_9, whole genome shotgun sequence |
| 510 | Microcystis aeruginosa PCC 9809 genomic scaffold, AAI_H_2202_scaffold61, |
| | whole genome shotgun sequence |
| 511 | Microcystis aeruginosa NIES-3804 DNA, sequence020, whole genome shotgun |
| | sequence |
| 512 | Scytonema sp. HK-05 NIES-2130_Scaffold_7, whole genome shotgun sequence |
| 513 | Candidatus Hydrothermarchaeota archaeon isolate B51_G15 |
| | B51_Guay15_scaffold_11912, whole genome shotgun sequence |
| 514 | Ga0373625_0000082 |
| 515 | Tolypothrix campylonemoides VB511288 scaffold_2, whole genome shotgun |
| | sequence |
| 516 | Ga0315294_10001891 |
| 517 | Microcystis wesenbergii Mw_QC_B_20070930_S4D S4D_10, whole genome |
| | shotgun sequence |
| 518 | Ga0373625_0034772 |
| 519 | Mastigocladus laminosus UU774 strain 74 scaffold_2, whole genome shotgun |
| | sequence |
| 520 | Moorea sp. SIO3I6 3I6_NODE_1, whole genome shotgun sequence |
| 521 | Planktothrix agardhii NIVA-CYA 56/3 contig00124, whole genome shotgun |
| | sequence |
| 522 | Nostoc sp. ATCC 53789 scaffold90.1, whole genome shotgun sequence |
| 523 | Symploca sp. SIO3C6 3C6_NODE_17, whole genome shotgun sequence |
| 524 | Microcystis flos-aquae DF17 NODE_180_length_69088_cov_289.53, whole genome |
| | shotgun sequence |
| 525 | Moorea sp. SIO3A2 3A2_NODE_11, whole genome shotgun sequence |
| 526 | Trichocoleus sp. FACHB-46 contig33, whole genome shotgun sequence |
| 527 | Planktothrix agardhii NIVA-CYA 34 contig00087, whole genome shotgun sequence |
| 528 | Arthrospira maxima CS-328 ctg6, whole genome shotgun sequence |
| 529 | Candidatus Poribacteria bacterium isolate PCPOR4 Ga0207198_1209, whole |
| | genome shotgun sequence |
| 530 | Candidatus Poribacteria bacterium WGA-3G POR3G_contig_6.7_C, whole genome |
| | shotgun sequence |
| 531 | uncultured cyanobacterium isolate I3_bin-238 genome assembly, contig: bin- |
| | 238: 681/866, whole genome shotgun sequence |
| 532 | Trichormus variabilis SAG 1403-4b sequence01, whole genome shotgun sequence |
| 533 | Ardenticatenales bacterium isolate MGR_bin47 SD2897-2912_k127_6180070, |
| | whole genome shotgun sequence |
| 534 | fermentation metagenome genome assembly, contig: |
| | NODE_485_length_50386_cov_69.906360, whole genome shotgun sequence |
| 535 | Spirulina subsalsa PCC 9445 Contig210_C2, whole genome shotgun sequence |
| 536 | Coleofasciculus chthonoplastes PCC 7420 ctg_1103659003746, whole genome |
| | shotgun sequence |
| 537 | Candidatus Poribacteria bacterium isolate SB0663_bin_6 |
| | NODE_4644_length_3525_cov_7.369452, whole genome shotgun sequence |
| 538 | Arthrospira platensis str. Paraca isolate UASWS Contig183, whole genome shotgun |
| | sequence |
| 539 | Ga0373629_0094687 |
| 540 | Ga0315277_10158218 |
| 541 | Gut metagenome scaffold37567_1, whole genome shotgun sequence |
| 542 | Richelia sp. SL_2_1 NODE_95_length_50065_cov_8.922143, whole genome |
| | shotgun sequence |
| 543 | Coleofasciculus chthonoplastes PCC 7420 ctg_1103659003746, whole genome |
| | shotgun sequence |
| 544 | Cyanobacteria bacterium QH_2_48_84 qh_2_scaffold_146, whole genome shotgun |
| | sequence |
| 545 | Symploca sp. SIO2C1 2C1_NODE_125, whole genome shotgun sequence |
| 546 | Ga0315279_10012021 |
| 547 | Crocosphaera watsonii WH 8502 WGS project CAQK00000000 data, contig 00305, |
| | whole genome shotgun sequence |
| 548 | Microcystis aeruginosa PCC 7005 Mic7005contig745, whole genome shotgun |
| | sequence |
| 549 | Oscillatoriales cyanobacterium isolate PH2015_12D_45_607 |
| | PH2015_12D_scaffold_712, whole genome shotgun sequence |
| 550 | Candidatus Chloroploca asiatica strain B7-9 |
| | NODE_14_length_96467_cov_78.3642_ID_198493, whole genome shotgun |
| | sequence |
| 551 | Ga0315296_10002743 |
| 552 | Microcystis wesenbergii FACHB-1317 contig99, whole genome shotgun sequence |
| 553 | Mastigocladopsis repens PCC 10914 Mas10914DRAFT_scaffold1.1_C5, whole |
| | genome shotgun sequence |
| 554 | Moorea producens 3L ctg45886, whole genome shotgun sequence |
| 555 | Ga0373628_0039306 |
| 556 | Acidithiobacillus thiooxidans strain CLST scaffold00011, whole genome shotgun |
| | sequence |
| 557 | Candidatus Poribacteria bacterium isolate Plut_88880 |
| | unfiltered_Plut_88880_genomic_contig_231928, whole genome shotgun sequence |
| 558 | Aphanothece sacrum FPU1 DNA, contig: ASFPU1_contig_001, whole genome |
| | shotgun sequence |
| 559 | Methanomicrobiales archaeon isolate AS21ysBPME_11 103734_AS21, whole |
| | genome shotgun sequence |
| 560 | Pleurocapsa sp. CCALA 161 NODE_15_length_65967_cov_11.0301_ID_47581, |
| | whole genome shotgun sequence |
| 561 | Candidatus Poribacteria bacterium isolate SB0663_bin_6 |
| | NODE_888_length_10879_cov_6.783814, whole genome shotgun sequence |
| 562 | Ga0315285_10002699 |
| 563 | Chloroflexi bacterium isolate CF_117 14_0929_09_20cm_scaffold_32114, whole |
| | genome shotgun sequence |
| 564 | Microcystis aeruginosa W11-06 Ga0188003_10305, whole genome shotgun |
| | sequence |
| 565 | TPA_asm: Synechococcus sp. isolate SpSt-285 Ga0101945_1000396, whole genome |
| | shotgun sequence |
| 566 | Oscillatoriales cyanobacterium isolate PH2015_07U_46_236 |
| | PH2015_07U_scaffold_20, whole genome shotgun sequence |
| 567 | coral metagenome genome assembly, contig: |
| | NODE_34_length_47446_cov_16.642467, whole genome shotgun sequence |
| 568 | Anabaena sp. PCC 7108 Ana7108scaffold_2_Cont3, whole genome shotgun |
| | sequence |
| 569 | Anabaena sp. CA = ATCC 33047 contig081, whole genome shotgun sequence |
| 570 | Geitlerinema sp. PCC 7105 genomic scaffold Gei7105DRAFT_GPC.4, whole |
| | genome shotgun sequence |
| 571 | Planktothrix agardhii NIVA-CYA 56/3 contig00014, whole genome shotgun |
| | sequence |
| 572 | Leptolyngbya boryana dg5 plasmid pLBX DNA, complete genome, strain: dg5 |
| 573 | bioreactor sludge metagenome genome assembly, contig: |
| | NODE_19_length_148633_cov_33.775869, whole genome shotgun sequence |
| 574 | Calothrix sp. FACHB-168 contig13, whole genome shotgun sequence |
| 575 | Oscillatoriales cyanobacterium isolate PH2015_09S_45_247 |
| | PH2015_09S_scaffold_4, whole genome shotgun sequence |
| 576 | Calothrix sp. CSU_2_0 NODE_2568_length_6893_cov_4.74682, whole genome |
| | shotgun sequence |
| 577 | Aliterella atlantica CENA595 contig_243, whole genome shotgun sequence |
| 578 | Oscillatoriales cyanobacterium RU_3_3 NODE_1980_length_14615_cov_2.286996, |
| | whole genome shotgun sequence |
| 579 | Microcystis aeruginosa NIES-88 scaffold3, whole genome shotgun sequence |
| 580 | Viral metagenome NODE_2825_length_39530_cov_3.55673, whole genome |
| | shotgun sequence |
| 581 | Acaryochloris sp. CCMEE 5410 contig00452, whole genome shotgun sequence |
| 582 | TPA_asm: Cyanobacteria bacterium UBA11370 contig_1246, whole genome |
| | shotgun sequence |
| 583 | Freshwater metagenome, whole genome shotgun sequence |
| 584 | Microcoleus sp. SU_5_6 NODE_1147_length_10793_cov_2.600975, whole genome |
| | shotgun sequence |
| 585 | Candidatus Poribacteria bacterium isolate SB0663_bin 6 |
| | NODE_1215_length_9091_cov_6.427291, whole genome shotgun sequence |
| 586 | Mastigocladopsis repens PCC 10914 Mas10914DRAFT_scaffold1.1_C9, whole |
| | genome shotgun sequence |
| 587 | metagenome genome assembly, contig: NODE_2330_length_23654_cov_8.032544, |
| | whole genome shotgun sequence |
| 588 | Symploca sp. SIO3C6 3C6_NODE_23, whole genome shotgun sequence |
| 589 | Microcystis wesenbergii Mw_QC_B_20070930_S4 S4_59, whole genome shotgun |
| | sequence |
| 590 | Ga0315277_10009605 |
| 591 | Oscillatoriales cyanobacterium isolate PH2015_07U_46_236 |
| | PH2015_07U_scaffold_61, whole genome shotgun sequence |
| 592 | Methanocalculus sp. isolate CSSed165cm_604R1 CSSed16-5cm-421, whole genome |
| | shotgun sequence |
| 593 | Cyanobacteria bacterium QH_10_48_56 qh_10_scaffold_929, whole genome |
| | shotgun sequence |
| 594 | Microcystis aeruginosa Ma_SC_T_19800800_S464 S464_89, whole genome |
| | shotgun sequence |
| 595 | Mastigocladus laminosus UU774 scaffold_3, whole genome shotgun sequence |
| 596 | Microcystis panniformis Mp_MB_F_20080800_S26D S26D_11, whole genome |
| | shotgun sequence |
| 597 | Oscillatoriales cyanobacterium isolate PH2015_07U_46_236 |
| | PH2015_07U_scaffold_188, whole genome shotgun sequence |
| 598 | Ga0373627_0079985 |
| 599 | Mastigocladus laminosus UU774 scaffold_4, whole genome shotgun sequence |
| 600 | Moorea sp. SIO1G6 1G6_NODE_49, whole genome shotgun sequence |
| 601 | Geitlerinema sp. PCC 7105 Gei7105DRAFT_GPC.5_C44, whole genome shotgun |
| | sequence |
| 602 | Arthrospira sp. TJSD091 Contig142, whole genome shotgun sequence |
| 603 | Limnoraphis robusta CS-951 contig101, whole genome shotgun sequence |
| 604 | Microcystis aeruginosa KW Contig7, whole genome shotgun sequence |
| 605 | Planktothrix sp. PCC 11201 isolate BBR_PRJEB10991 genome assembly, contig: |
| | BBR_D_PL11201_Contig_65, whole genome shotgun sequence |
| 606 | Tolypothrix [Scytonema hofmanni] UTEX 2349 genomic scaffold |
| | Tol9009DRAFT_TPD.5, whole genome shotgun sequence |
| 607 | Oscillatoriales cyanobacterium isolate PH2015_11S_45_847 |
| | PH2015_11S_scaffold_259, whole genome shotgun sequence |
| 608 | Microcoleus chthonoplastes PCC 7420 scf_1103659003829 genomic scaffold, whole |
| | genome shotgun sequence |
| 609 | Thermodesulfitimonas autotrophica strain DSM 102936 Ga0244728_11, whole |
| | genome shotgun sequence |
| 610 | Pleurocapsa sp. CCALA 161 NODE_10_length_78268_cov_10.6832_ID_51584, |
| | whole genome shotgun sequence |
| 611 | Crocosphaera watsonii WH0402 WGS project CAQN00000000 data, contig 01240, |
| | whole genome shotgun sequence |
| 612 | Cyanobacteria bacterium QH_9_48_43 qh_9_scaffold_2265, whole genome shotgun |
| | sequence |
| 613 | Crocosphaera watsonii WH 0401 WGS project CAQM00000000 data, contig 01078, |
| | whole genome shotgun sequence |
| 614 | Richelia sp. SM2_1_7 NODE_5681_length_4369_cov_1.798916, whole genome |
| | shotgun sequence |
| 615 | uncultured cyanobacterium isolate B12_bin-0982 genome assembly, contig: bin- |
| | 0982: 106/268, whole genome shotgun sequence |
| 616 | Geitlerinema sp. PCC 9228 Ga0115370_1196, whole genome shotgun sequence |
| 617 | Ga0315284_10104187 |
| 618 | Moorea sp. SIO1G6 1G6_NODE_1, whole genome shotgun sequence |
| 619 | Moorea sp. SIO3E2 3E2_NODE_539, whole genome shotgun sequence |
| 620 | Richelia sp. SM2_1_7 NODE_5681_length_4369_cov_1.798916, whole genome |
| | shotgun sequence |
| 621 | Planktothrix sp. PCC 11201 isolate BBR_PRJEB10991 genome assembly, scaffold: |
| | BBR_D_PL11201_scaffold28, whole genome shotgun sequence |
| 622 | Lyngbya majuscula 3L genomic scaffold scf52036, whole genome shotgun sequence |
| 623 | Chloroflexi bacterium isolate CFX10 c_000000000001, whole genome shotgun |
| | sequence |
| 624 | Microcystis aeruginosa PCC 9808 genomic scaffold, AAI_G_2201_scaffold9, whole |
| | genome shotgun sequence |
| 625 | Synechococcus sp. PCC 7335 scf_1103496006892 genomic scaffold, whole genome |
| | shotgun sequence |
| 626 | Microcystis aeruginosa NIES-4264 DNA, contig_1, whole genome shotgun sequence |
| 627 | Spirulina subsalsa PCC 9445 Contig210_C1, whole genome shotgun sequence |
| 628 | Candidatus Poribacteria bacterium isolate SB0662_bin_49 |
| | NODE_165_length_109673_cov_85.601252, whole genome shotgun sequence |
| 629 | Candidatus Poribacteria bacterium isolate PCPOR4 Ga0207198_1173, whole |
| | genome shotgun sequence |
| 630 | Candidatus Poribacteria bacterium isolate PCPOR2b Ga0206366_127, whole |
| | genome shotgun sequence |
| 631 | Anabaena sp. AL09 isolate 39864, whole genome shotgun sequence |
| 632 | Chloroflexi bacterium 13_1_20CM_54_36 13_1_20cm_full_scaffold_849, whole |
| | genome shotgun sequence |
| 633 | Coleofasciculus chthonoplastes PCC 7420 ctg_1103659003710, whole genome |
| | shotgun sequence |
| 634 | Planktothrix prolifica NIVA-CYA 540 contig00076, whole genome shotgun |
| | sequence |
| 635 | Nostoc sp. PCC 7120 sequence036, whole genome shotgun sequence |
| 636 | Ga0373625_0200948 |
| 637 | Oscillatoriales cyanobacterium isolate PH2015_08U_46_180 |
| | PH2015_08U_scaffold 548, whole genome shotgun sequence |
| 638 | Acaryochloris sp. CCMEE 5410 contig00452, whole genome shotgun sequence |
| 639 | Ga0315294_10001984 |
| 640 | Microcoleus chthonoplastes PCC 7420 scf_1103659003820 genomic scaffold, whole |
| | genome shotgun sequence |
| 641 | Chloroflexaceae bacterium isolate SM1_1_0 |
| | NODE_2191_length_12172_cov_8.956496, whole genome shotgun sequence |
| 642 | Calditerricola satsumensis JCM 14719 DNA, sequence008, whole genome shotgun |
| | sequence |
| 643 | Fischerella sp. PCC 9605 FIS9605DRAFT_scaffold12.12_C, whole genome shotgun |
| | sequence |
| 644 | Nostoc sp. PCC 7120 plasmid pCC7120alpha DNA, complete genome |
| 645 | Calothrix desertica PCC 7102 sequence007, whole genome shotgun sequence |
| 646 | Candidatus Poribacteria bacterium isolate PCPOR1 Ga0206387_1074, whole |
| | genome shotgun sequence |
| 647 | Coleofasciculus chthonoplastes PCC 7420 ctg_1103659003737, whole genome |
| | shotgun sequence |
| 648 | Moorea sp. SIO3G5 3G5_NODE_1005, whole genome shotgun sequence |
| 649 | Candidatus Poribacteria bacterium isolate SB0662_bin_35 |
| | NODE_11069_length_4700_cov_42.477287, whole genome shotgun sequence |
| 650 | Planktothrix agardhii NIVA-CYA 56/3 genomic scaffold scaffold00008, whole |
| | genome shotgun sequence |
| 651 | Microcoleus chthonoplastes PCC 7420 scf_1103659003831 genomic scaffold, whole |
| | genome shotgun sequence |
| 652 | Nostoc sp. ATCC 53789 plasmid pNsp_b, complete sequence |
| 653 | Scytonema sp. UIC 10036 c00161_NODE_16 . . . , whole genome shotgun sequence |
| 654 | Candidatus Poribacteria bacterium isolate PCPOR4 Ga0207198_1210, whole |
| | genome shotgun sequence |
| 655 | Ga0373629_0012366 |
| 656 | Ga0315275_10046509 |
| 657 | Richelia sp. CSU_2_1 NODE_124_length_49110_cov_7.99549, whole genome |
| | shotgun sequence |
| 658 | Leptolyngbya boryana PCC 6306 genomic scaffold LepboDRAFT_LPC.2, whole |
| | genome shotgun sequence |
| 659 | Anabaena sp. AL93 isolate WA93 101, whole genome shotgun sequence |
| 660 | Microcoleus sp. FACHB-DQ6 contig136, whole genome shotgun sequence |
| 661 | Microcoleus chthonoplastes PCC 7420 scf_1103659003817 genomic scaffold, whole |
| | genome shotgun sequence |
| 662 | Scytonema sp. UIC 10036 c00181_NODE_18 . . . , whole genome shotgun sequence |
| 663 | Ga0373630_0033251 |
| 664 | Microcoleus sp. SU_5_3 NODE_1012_length_11577_cov_3.501223, whole genome |
| | shotgun sequence |
| 665 | TPA_asm: Methanomicrobia archaeon isolate HyVt-11 Ga0116201_100544, whole |
| | genome shotgun sequence |
| 666 | [Scytonema hofmanni] UTEX 2349 Tol9009DRAFT_TPD.8_C61, whole genome |
| | shotgun sequence |
| 667 | Oscillatoriales cyanobacterium isolate PH2015_08U_46_180 |
| | PH2015_08U_scaffold_312, whole genome shotgun sequence |
| 668 | Moorea sp. SIO3C2 3C2_NODE_58, whole genome shotgun sequence |
| 669 | Bacterium isolate NIOZ-UU8 NIOZ-UU_NODE_2519_length_23800_cov_8.3314, |
| | whole genome shotgun sequence |
| 670 | Microcystis aeruginosa PCC 9717 genomic scaffold, AAI_B_2196_scaffold3, whole |
| | genome shotgun sequence |
| 671 | Candidatus Poribacteria bacterium bin44 |
| | NODE_3295_length_21674_cov_18.9376_ID_6589, whole genome shotgun |
| | sequence |
| 672 | Calothrix sp. NIES-4105 plasmid plasmid1 DNA, complete genome |
| 673 | Mastigocladus laminosus UU774 scaffold_14, whole genome shotgun sequence |
| 674 | Ga0373630_0000125 |
| 675 | Crocosphaera watsonii WH 8501 ctg230, whole genome shotgun sequence |
| 676 | Lyngbya sp. PCC 8106 1099428180525, whole genome shotgun sequence |
| 677 | Cyanobacteria bacterium RU_5_0 NODE_647_length_26982_cov_14.272575, |
| | whole genome shotgun sequence |
| 678 | Oscillatoriales cyanobacterium isolate PH2015_12U_45_315 |
| | PH2015_12U_scaffold_146, whole genome shotgun sequence |
| 679 | bioreactor sludge metagenome genome assembly, contig: |
| | NODE_312_length_7818_cov_3.276826, whole genome shotgun sequence |
| 680 | Merismopedia sp. SIO2A8 2A8_NODE_240, whole genome shotgun sequence |
| 681 | Microcystis aeruginosa NIES-2520 DNA, contig_62, whole genome shotgun |
| | sequence |
| 682 | Ga0315284_10054593 |
| 683 | Trichocoleus sp. FACHB-40 contig64, whole genome shotgun sequence |
| 684 | Brasilonema bromeliae SPC951 NODE_3533_length_9450_cov_4.016285, whole |
| | genome shotgun sequence |
| 685 | Cyanobacteria bacterium QS_8_48_54 qs_8_scaffold_4292, whole genome shotgun |
| | sequence |
| 686 | Merismopedia glauca CCAP 1448/3 |
| | NODE_172_length_31568_cov_6.44124_ID_38037, whole genome shotgun |
| | sequence |
| 687 | Mastigocladus laminosus UU774 strain 74 scaffold_4, whole genome shotgun |
| | sequence |
| 688 | uncultured Microcoleus sp. isolate AVDCRST_MAG84 genome assembly, contig: |
| | NODE360, whole genome shotgun sequence |
| 689 | Lyngbya majuscula 3L genomic scaffold scf49145, whole genome shotgun sequence |
| 690 | Microcystis aeruginosa BLCCF158 A6_contig_72, whole genome shotgun sequence |
| 691 | Oscillatoriales cyanobacterium isolate PH2015_01D_46_31 |
| | PH2015_01D_scaffold_1233, whole genome shotgun sequence |
| 692 | Ktedonobacter sp. 13_2_20CM_2_56_8 13_2_20cm_2_scaffold_552, whole genome |
| | shotgun sequence |
| 693 | Microcystis aeruginosa Ma_MB_S_20031200_S102 S102_57, whole genome |
| | shotgun sequence |
| 694 | Symploca sp. SIO2B6 2B6_NODE_6, whole genome shotgun sequence |
| 695 | Microcystis panniformis Mp_MB_F_20080800_S26 S26_24, whole genome shotgun |
| | sequence |
| 696 | Microcystis aeruginosa NIES-87 DNA, sequence016, whole genome shotgun |
| | sequence |
| 697 | Moorea sp. SIO4A3 4A3_NODE_299, whole genome shotgun sequence |
| 698 | Tolypothrix sp. T3-bin4 k141_222205_length_2371_cov_6.0000, whole genome |
| | shotgun sequence |
| 699 | Planktothrix prolifica NIVA-CYA 98 contig00182, whole genome shotgun sequence |
| 700 | Oscillatoriales cyanobacterium isolate PH2015_07D_46_1245 |
| | PH2015_07D_scaffold_1653, whole genome shotgun sequence |
| 701 | Microcoleus sp. FACHB-61 contig80, whole genome shotgun sequence |
| 702 | Candidatus Poribacteria bacterium isolate SB0675_bin_22 |
| | NODE_226_length_59321_cov_11.232410, whole genome shotgun sequence |
| 703 | Oscillatoriales cyanobacterium isolate PH2015_07D_46_1245 |
| | PH2015 07D_scaffold_1598, whole genome shotgun sequence |
| 704 | Planktothrix rubescens strain PCC 7821 genome assembly, contig: |
| | AAI_N_PLAN_Contig_12, whole genome shotgun sequence |
| 705 | Crocosphaera watsonii WH 0005 WGS project CAQL00000000 data, contig 00096, |
| | whole genome shotgun sequence |
| 706 | Fischerella sp. isolate L.E.CL.63 k119_1170006, whole genome shotgun sequence |
| 707 | Ga0315279_10074524 |
| 708 | Crocosphaera watsonii WH 8501 ctg230, whole genome shotgun sequence |
| 709 | Cyanobacteria bacterium QS_5_48_63 qs_5_scaffold_1134, whole genome shotgun |
| | sequence |
| 710 | Gloeocapsa sp. PCC 73106 scaffold_00137, whole genome shotgun sequence |
| 711 | Chloroflexi bacterium isolate CF_156 14_0903_12_20cm_scaffold_15515, whole |
| | genome shotgun sequence |
| 712 | Dolichospermum sp. UHCC 0259 scaffold237_cov0, whole genome shotgun |
| | sequence |
| 713 | Limnoraphis robusta CS-951 contig101, whole genome shotgun sequence |
| 714 | Nostoc sp. ATCC 53789 scaffold90.1, whole genome shotgun sequence |
| 715 | Scytonema sp. NIES-4073 plasmid plasmid4 DNA, complete genome |
| 716 | marine sediment metagenome genome assembly, contig: |
| | NODE_472_length_12067_cov_23.972361, whole genome shotgun sequence |
| 717 | Microcystis panniformis Mp_GB_SS_20050300_S99D S99D_290, whole genome |
| | shotgun sequence |
| 718 | Leptolyngbya sp. FACHB-161 contig17, whole genome shotgun sequence |
| 719 | Mastigocladus laminosus UU774 scaffold_2, whole genome shotgun sequence |
| 720 | Microcoleus chthonoplastes PCC 7420 scf_1103659003824 genomic scaffold, whole |
| | genome shotgun sequence |
| 721 | Marine metagenome k99_9634682, whole genome shotgun sequence |
| 722 | Cyanobacteria bacterium isolate GSL.Bin1 |
| | NODE_11831_length_5427_cov_5.19506, whole genome shotgun sequence |
| 723 | Ga0373629_0000913 |
| 724 | Symploca sp. SIO2E6 2E6_NODE_166, whole genome shotgun sequence |
| 725 | Arthrospira maxima CS-328 ctg6, whole genome shotgun sequence |
| 726 | Coleofasciculus chthonoplastes PCC 7420 ctg_1103659003716, whole genome |
| | shotgun sequence |
| 727 | Moorea sp. SIO4G3 4G3_NODE_88, whole genome shotgun sequence |
| 728 | Microcoleus sp. SM1_3_4 NODE_9942_length_4243_cov_2.146987, whole genome |
| | shotgun sequence |
| 729 | Calothrix elsteri CCALA 953 scaffold1, whole genome shotgun sequence |
| 730 | Scytonema millei VB511283 scaffold_83, whole genome shotgun sequence |
| 731 | Ga0373628_0125242 |
| 732 | uncultured cyanobacterium isolate I3b_bin-053 genome assembly, contig: bin- |
| | 053: 0899/1000, whole genome shotgun sequence |
| 733 | Ga0373628_0023257 |
| 734 | Tychonema bourrellyi FEM_GT703 scaffold98_size18864, whole genome shotgun |
| | sequence |
| 735 | cyanobacterium TDX16 genomic scaffold Scaffold113, whole genome shotgun |
| | sequence |
| 736 | Oscillatoriales cyanobacterium isolate PH2015_06S_46_2585 |
| | PH2015_06S_scaffold_5205, whole genome shotgun sequence |
| 737 | Microcoleus sp. Co-bin12 k141_18383957_length_3551_cov_15.0000, whole |
| | genome shotgun sequence |
| 738 | Ga0373625_0150781 |
| 739 | Microcystis aeruginosa NIES-2522 DNA, contig_16, whole genome shotgun |
| | sequence |
| 740 | Merismopedia sp. SIO2A8 2A8_NODE_178, whole genome shotgun sequence |
| 741 | Thermodesulfitimonas autotrophica strain DSM 102936 Ga0244728_11, whole |
| | genome shotgun sequence |
| 742 | Planktothrix agardhii NIVA-CYA 34 contig00003, whole genome shotgun sequence |
| 743 | Scytonema hofmannii FACHB-248 contig65, whole genome shotgun sequence |
| 744 | Microcystis aeruginosa NIES-2522 DNA, contig_11, whole genome shotgun |
| | sequence |
| 745 | Ga0373625_0019996 |
| 746 | uncultured cyanobacterium isolate A5_bin-0177 genome assembly, contig: bin- |
| | 0177: 194/358, whole genome shotgun sequence |
| 747 | Candidatus Poribacteria bacterium isolate Plut_88891 |
| | unfiltered_Plut_88891_genomic_contig_791289, whole genome shotgun sequence |
| 748 | Okeania sp. KiyG1 DNA, sequence087, whole genome shotgun sequence |
| 749 | Sponge metagenome NODE_682_length_63463_cov_19.1244_ID_1363, whole |
| | genome shotgun sequence |
| 750 | Candidatus Poribacteria bacterium bin44 |
| | NODE_10808_length_4286_cov_13.0202_ID_21615, whole genome shotgun |
| | sequence |
| 751 | Oscillatoriales cyanobacterium isolate PH2015_08U_46_180 |
| | PH2015_08U_scaffold_525, whole genome shotgun sequence |
| 752 | Anabaena variabilis FACHB-164 contig6, whole genome shotgun sequence |
| 753 | Sponge metagenome NODE_8534_length_9264_cov_3.88257_ID_17067, whole |
| | genome shotgun sequence |
| 754 | Coleofasciculus chthonoplastes PCC 7420 ctg_1103659003657, whole genome |
| | shotgun sequence |
| 755 | Planktothrix sp. FACHB-1355 contig815, whole genome shotgun sequence |
| 756 | Coleofasciculus chthonoplastes PCC 7420 ctg_1103659003710, whole genome |
| | shotgun sequence |
| 757 | Candidatus Poribacteria bacterium isolate SB0678_bin_11 |
| | NODE_981_length_15456_cov_6.404649, whole genome shotgun sequence |
| 758 | Scytonema sp. HK-05 NIES-2130_Scaffold_4, whole genome shotgun sequence |
| 759 | Microcystis aeruginosa K13-06 Ga0188025_103771, whole genome shotgun |
| | sequence |
| 760 | Nostoc sp. 106C 19_117334_26.4148_51_117334_0.395332981062608, whole |
| | genome shotgun sequence |
| 761 | Oscillatoriales cyanobacterium RU_3_3 NODE_12232_length_4241_cov_2.991979, |
| | whole genome shotgun sequence |
| 762 | Microcoleus sp. bin48.metabat.b7b8b9.023 WN_Microcoleus_1_scaffold_74, whole |
| | genome shotgun sequence |
| 763 | Ga0315279_10000292 |
| 764 | Crocosphaera watsonii WH 8502 WGS project CAQK00000000 data, contig 00461, |
| | whole genome shotgun sequence |
| 765 | Sponge metagenome NODE_20809_length_4413_cov_12.934_ID_41617, whole |
| | genome shotgun sequence |
| 766 | Candidatus Poribacteria bacterium isolate SB0678_bin_11 |
| | NODE_2683_length_8095_cov_7.303607, whole genome shotgun sequence |
| 767 | Mastigocladus laminosus UU774 scaffold_40, whole genome shotgun sequence |
| 768 | Dolichospermum sp. UHCC 0406 scaffold7_cov325, whole genome shotgun |
| | sequence |
| 769 | fermentation metagenome genome assembly, contig: |
| | NODE_6575_length_8110_cov_4.774426, whole genome shotgun sequence |
| 770 | Moorea sp. SIO3F7 3F7_NODE_36, whole genome shotgun sequence |
| 771 | Ga0373628_0056463 |
| 772 | Candidatus Poribacteria bacterium isolate SB0662_bin_35 |
| | NODE_2684_length_18948_cov_37.344890, whole genome shotgun sequence |
| 773 | [Scytonema hofmanni] UTEX 2349 Tol9009DRAFT_TPD.5_C3, whole genome |
| | shotgun sequence |
| 774 | Microcystis aeruginosa NIES-4264 DNA, contig_1, whole genome shotgun sequence |
| 775 | Candidatus Methanoculleus thermohydrogenotrophicum isolate DTU006 |
| | scaffold603, whole genome shotgun sequence |
| 776 | Candidatus Poribacteria bacterium isolate PCPOR2 Ga0207196_171, whole genome |
| | shotgun sequence |
| 777 | Oscillatoriales cyanobacterium isolate PH2015_01U_44_212 |
| | PH2015 01U_scaffold_598, whole genome shotgun sequence |
| 778 | Ga0255812_10666830 |
| 779 | Oscillatoriales cyanobacterium isolate PH2015_04D_45_965 |
| | PH2015_04D_scaffold_577, whole genome shotgun sequence |
| 780 | Candidatus Poribacteria bacterium isolate SB0669_bin_10 |
| | NODE_735_length_15227_cov_8.792381, whole genome shotgun sequence |
| 781 | viral metagenome genome assembly, contig: k249_576930, whole genome shotgun |
| | sequence |
| 782 | Okeania sp. SIO2D1 2D1_NODE_2565, whole genome shotgun sequence |
| 783 | Ga0373629_0010383 |
| 784 | TPA_asm: Synechococcus sp. isolate SpSt-164 Ga0101943_1001739, whole genome |
| | shotgun sequence |
| 785 | Candidatus Poribacteria bacterium isolate PCPOR1 Ga0206387_1065, whole |
| | genome shotgun sequence |
| 786 | Moorea sp. SIO3A5 3A5_NODE_1, whole genome shotgun sequence |
| 787 | Candidatus Poribacteria bacterium isolate SB0661_bin_50 |
| | NODE_4170_length_10283_cov_10.155749, whole genome shotgun sequence |
| 788 | Crocosphaera watsonii WH 8502 WGS project CAQK00000000 data, contig 00002, |
| | whole genome shotgun sequence |
| 789 | Microcystis aeruginosa NIES-2522 DNA, contig_18, whole genome shotgun |
| | sequence |
| 790 | Candidatus Poribacteria bacterium isolate PCPOR1 Ga0206387_1076, whole |
| | genome shotgun sequence |
| 791 | metagenomes genome assembly, contig: NODE_4567_length_4333_cov_2.149369, |
| | whole genome shotgun sequence |
| 792 | Dolichospermum sp. UHCC 0259 scaffold275_cov0, whole genome shotgun |
| | sequence |
| 793 | Cyanobacteria bacterium SW_8_48_13 sw_8_scaffold_613, whole genome shotgun |
| | sequence |
| 794 | Moorea sp. SIO3I8 3I8_NODE_130, whole genome shotgun sequence |
| 795 | Cyanobacteria bacterium QH_1_48_107 qh_1_scaffold_1498, whole genome |
| | shotgun sequence |
| 796 | Mastigocladopsis repens PCC 10914 Mas10914DRAFT_scaffold1.1_C11, whole |
| | genome shotgun sequence |
| 797 | Microcystis aeruginosa PCC 9717 genomic scaffold, AAI_B_2196_scaffold107, |
| | whole genome shotgun sequence |
| 798 | Microcystis aeruginosa BS13-02 Ga0188020_100328, whole genome shotgun |
| | sequence |
| 799 | Moorea sp. SIO3I8 3I8_NODE_43, whole genome shotgun sequence |
| 800 | Calothrix sp. PCC 7103 Cal7103DRAFT_CPM.1_C5, whole genome shotgun |
| | sequence |
| 801 | Ga0315284_10005110 |
| 802 | Kouleothrix aurantiaca strain COM-B contig_153, whole genome shotgun sequence |
| 803 | Oscillatoriales cyanobacterium isolate PH2015_03D_45 235 |
| | PH2015_03D_scaffold_234, whole genome shotgun sequence |
| 804 | Moorea sp. SIO2B7 2B7_NODE_1581, whole genome shotgun sequence |
| 805 | Nostoc linckia FACHB-104 contig34, whole genome shotgun sequence |
| 806 | Okeania sp. SIO3I5 3I5_NODE_203, whole genome shotgun sequence |
| 807 | TPA_asm: Pricia sp. isolate HyVt-320 HyVt-320_k145_146519, whole genome |
| | shotgun sequence |
| 808 | Oscillatoriales cyanobacterium isolate PH2015_04U_45_1042 |
| | PH2015_04U_scaffold_439, whole genome shotgun sequence |
| 809 | Ga0315280_10014910 |
| 810 | Microcoleus chthonoplastes PCC 7420 scf_1103659003820 genomic scaffold, whole |
| | genome shotgun sequence |
| 811 | Oscillatoriales cyanobacterium isolate PH2015_06S_46_2585 |
| | PH2015_06S_scaffold_8789, whole genome shotgun sequence |
| 812 | Ga0315280_10062518 |
| 813 | Ga0315277_10071052 |
| 814 | Microcystis aeruginosa W11-03 Ga0187975_10118, whole genome shotgun |
| | sequence |
| 815 | Lyngbya majuscula 3L genomic scaffold scf52117, whole genome shotgun sequence |
| 816 | Microcystis aeruginosa BLCCF158 A6_contig_33, whole genome shotgun sequence |
| 817 | Candidatus Gracilibacteria bacterium isolate SU_1_2 |
| | NODE_5_length_107249_cov_3.991104, whole genome shotgun sequence |
| 818 | Chlorogloeopsis fritschii PCC 6912 sequence18, whole genome shotgun sequence |
| 819 | Anabaena minutissima FACHB-250 contig50, whole genome shotgun sequence |
| 820 | Ga0373625_0208636 |
| 821 | Bacterium isolate NIOZ-UU8 NIOZ-UU_NODE_2597_length_23367_cov_8.29328, |
| | whole genome shotgun sequence |
| 822 | Candidatus Poribacteria bacterium bin44 |
| | NODE_3467_length_20673_cov_18.9015_ID_6933, whole genome shotgun |
| | sequence |
| 823 | Ga0373625_0000556 |
| 824 | Candidatus Poribacteria bacterium isolate SB0663_bin_6 |
| | NODE_1215_length_9091_cov_6.427291, whole genome shotgun sequence |
| 825 | sediment metagenome genome assembly, contig: |
| | NODE_4_length_48544_cov_10.095073, whole genome shotgun sequence |
| 826 | Coleofasciculus sp. Co-bin14 k141_2600778_length_3864_cov_6.9162, whole |
| | genome shotgun sequence |
| 827 | Microcoleus chthonoplastes PCC 7420 scf_1103659003781 genomic scaffold, whole |
| | genome shotgun sequence |
| 828 | Candidatus Poribacteria bacterium isolate SB0669_bin_10 |
| | NODE_506_length_18523_cov_9.568984, whole genome shotgun sequence |
| 829 | Oscillatoriales cyanobacterium isolate PH2015_12D_45_607 |
| | PH2015_12D_scaffold_284, whole genome shotgun sequence |
| 830 | Ga0315280_10062386 |
| 831 | wastewater metagenome genome assembly, contig: |
| | NODE_3775_length_6277_cov_3.468660, whole genome shotgun sequence |
| 832 | Cyanobacteria bacterium SW_10_48_33 sw_10_scaffold_2122, whole genome |
| | shotgun sequence |
| 833 | Sponge metagenome NODE_8241_length_9536_cov_20.0624_ID_16481, whole |
| | genome shotgun sequence |
| 834 | Brasilonema bromeliae SPC951 NODE_2367_length_14264_cov_4.685270, whole |
| | genome shotgun sequence |
| 835 | Oscillatoriales cyanobacterium isolate PH2015_10S_46_199 |
| | PH2015_10S_scaffold_127, whole genome shotgun sequence |
| 836 | Microcystis aeruginosa NIES-2522 DNA, contig_11, whole genome shotgun |
| | sequence |
| 837 | TPA_asm: Cyanobacteria bacterium UBA6047 UBA6047_contig_14, whole genome |
| | shotgun sequence |
| 838 | Ga0315294_10110587 |
| 839 | Okeania sp. SIO2D1 2D1_NODE_2565, whole genome shotgun sequence |
| 840 | Coleofasciculus chthonoplastes PCC 7420 ctg_1103659003737, whole genome |
| | shotgun sequence |
| 841 | Ga0315280_10000556 |
| 842 | Ga0373625_0069134 |
| 843 | Scytonema sp. HK-05 NIES-2130_Scaffold_7, whole genome shotgun sequence |
| 844 | Synechococcus sp. PCC 7335 scf_1103496006892 genomic scaffold, whole genome |
| | shotgun sequence |
| 845 | wastewater metagenome genome assembly, contig: |
| | NODE_103_length_54413_cov_6.362927, whole genome shotgun sequence |
| 846 | Microcystis sp. M_QC_C_20170808_M2Col M2Col_9, whole genome shotgun |
| | sequence |
| 847 | coral metagenome genome assembly, contig: |
| | NODE_19_length_33842_cov_7.533985, whole genome shotgun sequence |
| 848 | bioreactor sludge metagenome genome assembly, contig: |
| | NODE_10_length_82959_cov_115.220074, whole genome shotgun sequence |
| 849 | Chloroflexi bacterium isolate CF_154 14_0903_05_20cm_scaffold_1223, whole |
| | genome shotgun sequence |
| 850 | activated sludge metagenome genome assembly, contig: |
| | NODE_2679_length_5473_cov_4.070321, whole genome shotgun sequence |
| 851 | Moorea sp. SIO3C2 3C2_NODE_211, whole genome shotgun sequence |
| 852 | Microcystis wesenbergii Mw_QC_B_20070930_S4D S4D_39, whole genome |
| | shotgun sequence |
| 853 | Microcoleus chthonoplastes PCC 7420 scf_1103659003812 genomic scaffold, whole |
| | genome shotgun sequence |
| 854 | Coleofasciculus chthonoplastes PCC 7420 ctg_1103659003726, whole genome |
| | shotgun sequence |
| 855 | Oscillatoriales cyanobacterium isolate PH2015_03U_45_708 |
| | PH2015_03U_scaffold_1089, whole genome shotgun sequence |
| 856 | Spirulina subsalsa PCC 9445 Contig203_C, whole genome shotgun sequence |
| 857 | Oscillatoriales cyanobacterium isolate PH2015_02U_45_393 |
| | PH2015_02U_scaffold_88, whole genome shotgun sequence |
| 858 | Nostoc sp. FACHB-110 contig25, whole genome shotgun sequence |
| 859 | Ga0315273_10010493 |
| 860 | Ga0315279_10000059 |
| 861 | Microcoleus chthonoplastes PCC 7420 scf_1103659003831 genomic scaffold, whole |
| | genome shotgun sequence |
| 862 | Ga0315277_10058131 |
| 863 | Cyanobacteria bacterium SW_12_48_29 sw_12_scaffold_1078, whole genome |
| | shotgun sequence |
| 864 | Dolichospermum sp. UHCC 0259 scaffold275_cov0, whole genome shotgun |
| | sequence |
| 865 | Ga0373630_0041537 |
| 866 | Scytonema sp. HK-05 NIES-2130_Scaffold_6, whole genome shotgun sequence |
| 867 | Microcystis aeruginosa NIES-88 scaffold5, whole genome shotgun sequence |
| 868 | Ammonifex sp. isolate SURF_55 Ga0104751_1001767, whole genome shotgun |
| | sequence |
| 869 | Microcystis aeruginosa NIES-44 DNA, contig: contig1_21, strain: NIES-44, whole |
| | genome shotgun sequence |
| 870 | Oscillatoriales cyanobacterium RU_3_3 NODE_12232_length_4241_cov_2.991979, |
| | whole genome shotgun sequence |
| 871 | Scytonema sp. CRU_2_7 NODE_6006_length_4918_cov_2.50845, whole genome |
| | shotgun sequence |
| 872 | Okeania sp. SIO2D1 2D1_NODE_961, whole genome shotgun sequence |
| 873 | TPA_asm: Cyanobacteria bacterium UBA11372 contig_7805, whole genome |
| | shotgun sequence |
| 874 | Peptococcaceae bacterium isolate palsa_1188 73.20120500_P19.26_contig_2246, |
| | whole genome shotgun sequence |
| 875 | Candidatus Poribacteria bacterium isolate SB0675_bin_22 |
| | NODE_1437_length_17721_cov_14.118023, whole genome shotgun sequence |
| 876 | Cyanobacteria bacterium isolate GSL.Bin1 |
| | NODE_2522_length_16363_cov_5.19154, whole genome shotgun sequence |
| 877 | Calothrix sp. NIES-4071 plasmid plasmid1 DNA, complete genome |
| 878 | Calothrix elsteri CCALA 953 scaffold1, whole genome shotgun sequence |
| 879 | Microcoleus sp. Co-bin12 k141_18383957_length_3551_cov_15.0000, whole |
| | genome shotgun sequence |
| 880 | Microcystis aeruginosa W11-03 Ga0187975_10207, whole genome shotgun |
| | sequence |
| 881 | Sulfobacillus benefaciens isolate AMDSBA4 AMDSBA4_59, whole genome |
| | shotgun sequence |
| 882 | Ga0373629_0002383 |
| 883 | Arthrospira platensis NIES-46 DNA, sequence003, whole genome shotgun sequence |
| 884 | Calothrix sp. NIES-4071 plasmid plasmid2 DNA, complete genome |
| 885 | Acidithiobacillus ferrivorans WGS project CCCS000000000 data, strain CF27, |
| | contig ATN_AFERRI_Contig_8, whole genome shotgun sequence |
| 886 | Scytonema sp. CRU_2_7 NODE_6006_length_4918_cov_2.50845, whole genome |
| | shotgun sequence |
| 887 | Microcoleus sp. Co-bin12 k141_16798132_length_1964_cov_17.0000, whole |
| | genome shotgun sequence |
| 888 | Arthrospira platensis C1 scaffold06, whole genome shotgun sequence |
| 889 | Cyanothece sp. BG0011 unitig_31, whole genome shotgun sequence |
| 890 | Moorea sp. SIO3I7 3I7_NODE_59, whole genome shotgun sequence |
| 891 | TPA_asm: Deltaproteobacteria bacterium isolate UBA8905 contig_81556, whole |
| | genome shotgun sequence |
| 892 | Mastigocoleus testarum BC008 Contig-69, whole genome shotgun sequence |
| 893 | Iningainema sp. BLCCT55 NODE_25_length_141538_cov_25.362927, whole |
| | genome shotgun sequence |
| 894 | Candidatus Poribacteria bacterium isolate SB0661_bin_29 |
| | NODE_49_length_242868_cov_73.526133, whole genome shotgun sequence |
| 895 | Planktothrix agardhii NIVA-CYA 34 contig00087, whole genome shotgun sequence |
| 896 | Planktothrix prolifica NIVA-CYA 406 contig00144, whole genome shotgun |
| | sequence |
| 897 | Chlorogloeopsis fritschii PCC 6912 contig00059, whole genome shotgun sequence |
| 898 | Moorea sp. SIO4A5 4A5_NODE_16, whole genome shotgun sequence |
| 899 | Ga0315296_10000160 |
| 900 | Tolypothrix bouteillei VB521301 scaffold_8, whole genome shotgun sequence |
| 901 | Ga0373625_0000329 |
| 902 | Sponge metagenome NODE_4235_length_17335_cov_18.8799_ID_8469, whole |
| | genome shotgun sequence |
| 903 | Microcystis aeruginosa 9717 WGS project CAII01000000 data, contig |
| | AAI_B_2196_36, whole genome shotgun sequence |
| 904 | Moorea sp. SIO1G6 1G6_NODE_19, whole genome shotgun sequence |
| 905 | Scytonema sp. HK-05 NIES-2130_Scaffold_2, whole genome shotgun sequence |
| 906 | Cyanobacteria bacterium isolate GSL.Bin1 NODE_5738_length_9529_cov_5.21131, |
| | whole genome shotgun sequence |
| 907 | Ga0214071_1336518 |
| 908 | fermentation metagenome genome assembly, contig: |
| | NODE_581_length_39144_cov_26.091611, whole genome shotgun sequence |
| 909 | Ga0373626_0003683 |
| 910 | Ga0315294_10032635 |
| 911 | wastewater metagenome genome assembly, contig: |
| | NODE_6266_length_3913_cov_11.333852, whole genome shotgun sequence |
| 912 | Calothrix sp. NIES-4105 plasmid plasmid2 DNA, complete genome |
| 913 | Scytonema millei VB511283 scaffold_83, whole genome shotgun sequence |
| 914 | Microcystis aeruginosa NaRes975 Scaffold58_1, whole genome shotgun sequence |
| 915 | uncultured Syntrophaceae bacterium isolate AlinenSediments_bin-3900 genome |
| | assembly, contig: bin-3900: 172/742, whole genome shotgun sequence |
| 916 | Symploca sp. SIO1A3 1A3_NODE_235, whole genome shotgun sequence |
| 917 | Tychonema bourrellyi FEM_GT703 scaffold38_size30849, whole genome shotgun |
| | sequence |
| 918 | Dolichospermum sp. UHCC 0406 scaffold7_cov325, whole genome shotgun |
| | sequence |
| 919 | Moorea sp. SIO2C4 2C4_NODE_709, whole genome shotgun sequence |
| 920 | coral metagenome genome assembly, contig: |
| | NODE_200_length_14726_cov_17.158236, whole genome shotgun sequence |
| 921 | Arthrospira sp. PCC 8005, WGS project CAFN00000000 data, strain PCC 8005, |
| | Contig4958-2130, whole genome shotgun sequence |
| 922 | Ga0315296_10033857 |
| 923 | Acaryochloris marina MBIC11017 plasmid pREB2, complete sequence |
| 924 | Geitlerinema sp. PCC 7105 Gei7105DRAFT_GPC.5_C15, whole genome shotgun |
| | sequence |
| 925 | Calothrix parietina FACHB-288 contig3, whole genome shotgun sequence |
| 926 | human metagenome genome assembly, contig: |
| | NODE_749_length_32251_cov_16.9544, whole genome shotgun sequence |
| 927 | Ktedonobacter sp. 13_2_20CM_53_11 13_2_20cm_scaffold_115, whole genome |
| | shotgun sequence |
| 928 | Ga0315285_10087313 |
| 929 | Ga0373630_0000013 |
| 930 | Ga0307928_10006388 |
| 931 | Gloeocapsa sp. PCC 73106 scaffold_00213, whole genome shotgun sequence |
| 932 | Sponge metagenome NODE_14928_length_5778_cov_18.9207_ID_29855, whole |
| | genome shotgun sequence |
| 933 | uncultured cyanobacterium isolate B-1_bin-000 genome assembly, contig: bin- |
| | 000: 246/625, whole genome shotgun sequence |
| 934 | Moorea sp. SIO4E2 4E2_NODE_856, whole genome shotgun sequence |
| 935 | Moorea sp. SIO4G3 4G3_NODE_35, whole genome shotgun sequence |
| 936 | Moorea sp. SIO4A3 4A3_NODE_562, whole genome shotgun sequence |
| 937 | Scytonema sp. UIC 10036 c00074_NODE_74 . . . , whole genome shotgun sequence |
| 938 | Microcystis aeruginosa 9809 WGS project CAIO01000000 data, contig |
| | AAI_H_2202_430, whole genome shotgun sequence |
| 939 | Mastigocladus laminosus UU774 scaffold_2, whole genome shotgun sequence |
| 940 | Brasilonema sp. UFV-L1 scaffold102_cov13, whole genome shotgun sequence |
| 941 | Ga0315284_10111608 |
| 942 | Scytonema sp. HK-05 NIES-2130_Scaffold_97, whole genome shotgun sequence |
| 943 | Microcystis aeruginosa Ma_MB_S_20031200_S102D S102D_79, whole genome |
| | shotgun sequence |
| 944 | Stanieria cyanosphaera PCC 7437 plasmid pSTA7437.01, complete sequence |
| 945 | Aphanizomenon flos-aquae NIES-81 contig00159, whole genome shotgun sequence |
| 946 | Ga0315280_10064331 |
| 947 | Microcystis aeruginosa KW Contig7, whole genome shotgun sequence |
| 948 | Ga0315277_10008377 |
| 949 | Microcystis aeruginosa NIES-4264 DNA, contig_35, whole genome shotgun |
| | sequence |
| 950 | Okeania sp. SIO2D1 2D1_NODE_571, whole genome shotgun sequence |
| 951 | Ardenticatenales bacterium isolate MGR_bin47 SD2897-2912_k127_4773552, |
| | whole genome shotgun sequence |
| 952 | Clostridia bacterium isolate MAG-71 Moorellales_O3_bin30_Scaffold_52, whole |
| | genome shotgun sequence |
| 953 | Symploca sp. SIO2B6 2B6_NODE_34, whole genome shotgun sequence |
| 954 | Chamaesiphon polymorphus CCALA 037 |
| | NODE_187_length_14934_cov_6.09504_ID_66389, whole genome shotgun |
| | sequence |
| 955 | Scytonema sp. HK-05 NIES-2130_Scaffold_43, whole genome shotgun sequence |
| 956 | cyanobacterium TDX16 genomic scaffold Scaffold113, whole genome shotgun |
| | sequence |
| 957 | Moorea sp. SIO1F2 1F2_NODE_259, whole genome shotgun sequence |
| 958 | Candidatus Poribacteria bacterium isolate PCPOR4 Ga0207198_1002, whole |
| | genome shotgun sequence |
| 959 | Microcystis aeruginosa PCC 7941 genomic scaffold, AAI_D_2198_scaffold21, |
| | whole genome shotgun sequence |
| 960 | Planktothrix sp. FACHB-1375 contig194, whole genome shotgun sequence |
| 961 | Anaerobic digester metagenome 7356, whole genome shotgun sequence |
| 962 | Ga0315273_10032131 |
| 963 | Microcoleus sp. FACHB-53 contig6, whole genome shotgun sequence |
| 964 | Leptolyngbya foveolarum isolate ULC129bin1 scaffold6_size63472, whole genome |
| | shotgun sequence |
| 965 | uncultured Bacteroidetes bacterium isolate Umea3p4_bin-0112 genome assembly, |
| | contig: bin-0112: 053/751, whole genome shotgun sequence |
| 966 | Tychonema bourrellyi FEM_GT703 scaffold165_size8795, whole genome shotgun |
| | sequence |
| 967 | Dolichospermum sp. UHCC 0352 scaffold173_cov0, whole genome shotgun |
| | sequence |
| 968 | Ga0373625_0055246 |
| 969 | Planktothrix sp. FACHB-1375 contig120, whole genome shotgun sequence |
| 970 | Synechococcales cyanobacterium RM1_1_8 |
| | NODE_585_length_24958_cov_3.770368, whole genome shotgun sequence |
| 971 | Nitrosococcus oceani strain AFC132 contig_24, whole genome shotgun sequence |
| 972 | Ga0373626_0020143 |
| 973 | Microcystis aeruginosa F13-15 Ga0187998_10353, whole genome shotgun sequence |
| 974 | Candidatus Poribacteria bacterium isolate DGPOR9 scf7180000027709, whole |
| | genome shotgun sequence |
| 975 | Oscillatoriales cyanobacterium isolate PH2015_06S_46_2585 |
| | PH2015_06S_scaffold_6494, whole genome shotgun sequence |
| 976 | Moorea sp. SIO2I5_2I5_NODE_1458, whole genome shotgun sequence |
| 977 | Mastigocladus laminosus UU774 scaffold_3, whole genome shotgun sequence |
| 978 | Coleofasciculus chthonoplastes PCC 7420 ctg_1103659003736, whole genome |
| | shotgun sequence |
| 979 | TPA_asm: Richelia sp. UBA3958 UBA3958_contig_1375, whole genome shotgun |
| | sequence |
| 980 | Chroococcidiopsis sp. FACHB-1243 contig9, whole genome shotgun sequence |
| 981 | Burkholderiaceae bacterium isolate Bin_15_2 c_000000009312, whole genome |
| | shotgun sequence |
| 982 | Trichocoleus sp. FACHB-6 contig76, whole genome shotgun sequence |
| 983 | Scytonema sp. HK-05 NIES-2130_Scaffold 36, whole genome shotgun sequence |
| 984 | Crocosphaera watsonii WH0402 WGS project CAQN00000000 data, contig 01240, |
| | whole genome shotgun sequence |
| 985 | TPA_asm: Richelia sp. UBA3308 UBA3308_contig_795, whole genome shotgun |
| | sequence |
| 986 | Ga0315280_10060196 |
| 987 | Microcystis flos-aquae Mf_QC_C_20070823_S10D S10D_65, whole genome |
| | shotgun sequence |
| 988 | Pleurocapsa sp. CCALA 161 NODE_15_length_65967_cov_11.0301_ID_47581, |
| | whole genome shotgun sequence |
| 989 | Ga0373625_0205637 |
| 990 | Ga0315285_10000720 |
| 991 | Ga0315284_10006726 |
| 992 | Moorea sp. SIO4G2 4G2_NODE_2420, whole genome shotgun sequence |
| 993 | Chloroflexia bacterium isolate INTA.AUR.084 contig-100_52925, whole genome |
| | shotgun sequence |
| 994 | Planktothrix mougeotii NIVA-CYA 405 contig00063, whole genome shotgun |
| | sequence |
| 995 | Symploca sp. SIO2B6 2B6_NODE_6, whole genome shotgun sequence |
| 996 | Microcystis aeruginosa PCC 9809 genomic scaffold, AAI_H_2202_scaffold45, |
| | whole genome shotgun sequence |
| 997 | Okeania sp. SIO3I5 3I5_NODE_28, whole genome shotgun sequence |
| 998 | Leptolyngbya boryana NIES-2135 plasmid plasmid2 DNA, complete genome |
| 999 | Ga0315273_10103216 |
| 1000 | Chloroflexi bacterium isolate L227-5C MAG2_12, whole genome shotgun sequence |
| 1001 | Ga0315284_10065608 |
| 1002 | Microcystis aeruginosa NIES-88 scaffold5, whole genome shotgun sequence |
| 1003 | Candidatus Poribacteria bacterium isolate PCPOR4 Ga0207198_1058, whole |
| | genome shotgun sequence |
| 1004 | Ga0315295_10027354 |
| 1005 | Coleofasciculus chthonoplastes PCC 7420 ctg_1103659003742, whole genome |
| | shotgun sequence |
| 1006 | Ga0315294_10022559 |
| 1007 | Ga0315279_10047182 |
| 1008 | Cyanobacteria bacterium QH_6_48_35 qh_6_scaffold_2052, whole genome shotgun |
| | sequence |
| 1009 | Scytonema sp. UIC 10036 c00074_NODE_74 . . . , whole genome shotgun sequence |
| 1010 | Leptolyngbyaceae cyanobacterium RU_5_1 |
| | NODE_917_length_22282_cov_3.725254, whole genome shotgun sequence |
| 1011 | Scytonema sp. RU_4_4 NODE_236_length_45534_cov_15.648975, whole genome |
| | shotgun sequence |
| 1012 | Hydrocoleum sp. CS-953 1482, whole genome shotgun sequence |
| 1013 | Candidatus Poribacteria bacterium isolate SB0661_bin_29 |
| | NODE_309_length_90992_cov_74.217854, whole genome shotgun sequence |
| 1014 | Leptolyngbya sp. FACHB-238 contig17, whole genome shotgun sequence |
| 1015 | Microcystis phage MaMV-DC, complete genome |
| 1016 | Ga0315295_10001383 |
| 1017 | Candidatus Poribacteria bacterium bin44 |
| | NODE_9134_length_5559_cov_19.1806_ID_18267, whole genome shotgun |
| | sequence |
| 1018 | Richelia sp. CSU_2_1 NODE_121_length_49199_cov_8.4043, whole genome |
| | shotgun sequence |
| 1019 | Coleofasciculus chthonoplastes PCC 7420 ctg_1103659003743, whole genome |
| | shotgun sequence |
| 1020 | Sulfobacillus thermosulfidooxidans isolate AMDSBA5 AMDSBA5_98, whole |
| | genome shotgun sequence |
| 1021 | Ga0315277_10007795 |
| 1022 | Microcystis flos-aquae Mf_QC_C_20070823_S20D S20D_80, whole genome |
| | shotgun sequence |
| 1023 | Calditerricola satsumensis JCM 14719 DNA, sequence008, whole genome shotgun |
| | sequence |
| 1024 | Ga0373628_0134704 |
| 1025 | Microcystis aeruginosa NIES-88 scaffold3, whole genome shotgun sequence |
| 1026 | Calothrix sp. FACHB-1219 contig10, whole genome shotgun sequence |
| 1027 | Dolichospermum sp. UHCC 0406 scaffold19_cov315, whole genome shotgun |
| | sequence |
| 1028 | Ga0315285_10061643 |
| 1029 | Synechococcus sp. PCC 7335 ctg_1103496006870, whole genome shotgun sequence |
| 1030 | Cyanobacteria bacterium SW_6_48_11 sw_6_scaffold_1338, whole genome shotgun |
| | sequence |
| 1031 | Synechococcus sp. PCC 7335 ctg_1103496006867, whole genome shotgun sequence |
| 1032 | Planktothrix agardhii NIVA-CYA 56/3 contig00132, whole genome shotgun |
| | sequence |
| 1033 | Oscillatoriales cyanobacterium isolate PH2015_13D_45_19 |
| | PH2015_13D_scaffold_258, whole genome shotgun sequence |
| 1034 | TPA_asm: Cyanobacteria bacterium UBA8553 contig_3714, whole genome shotgun |
| | sequence |
| 1035 | Oscillatoriales cyanobacterium CG2_30_40_61 cg2_3.0_scaffold_1268_c, whole |
| | genome shotgun sequence |
| 1036 | Ga0315279_10000180 |
| 1037 | Coleofasciculus chthonoplastes PCC 7420 ctg_1103659003670, whole genome |
| | shotgun sequence |
| 1038 | Ga0315273_10129206 |
| 1039 | Dolichospermum sp. UHCC 0299 scaffold121_cov0, whole genome shotgun |
| | sequence |
| 1040 | Ga0315285_10084064 |
| 1041 | Prochlorothrix hollandica PCC 9006 = CALU 1027 strain PCC 9006 |
| | Pro9006DRAFT_Contig5.9_C24, whole genome shotgun sequence |
| 1042 | Oscillatoriales cyanobacterium isolate PH2015_08D_46_1646 |
| | PH2015_08D_scaffold_5805, whole genome shotgun sequence |
| 1043 | Symploca sp. SIO2E9 2E9_NODE_71, whole genome shotgun sequence |
| 1044 | Oscillatoriales cyanobacterium isolate PH2015_07D_45_11 |
| | PH2015_07D_scaffold_416, whole genome shotgun sequence |
| 1045 | Ga0373625_0078553 |
| 1046 | Aphanothece sacrum FPU3 DNA, contig: ASFPU3_contig081, whole genome |
| | shotgun sequence |
| 1047 | Coleofasciculus sp. C3-bin4 k141_1828533_length_9803_cov_7.0000, whole |
| | genome shotgun sequence |
| 1048 | Hydrococcus sp. CRU_1_1 NODE_214_length_36770_cov_3.82297, whole genome |
| | shotgun sequence |
| 1049 | Mastigocoleus testarum BC008 Contig-69, whole genome shotgun sequence |
| 1050 | Microcystis flos-aquae DF17 NODE_91_length_152052_cov_197.105, whole |
| | genome shotgun sequence |
| 1051 | Ga0373630_0000139 |
| 1052 | Sponge metagenome NODE_16204_length_5401_cov_3.76754_ID_32407, whole |
| | genome shotgun sequence |
| 1053 | Microcoleus sp. bin38.metabat.b11b12b14.051 WN_Microcoleus_2_scaffold_29, |
| | whole genome shotgun sequence |
| 1054 | Thermincola ferriacetica strain Z-0001 Tfer_ctg04, whole genome shotgun sequence |
| 1055 | Ga0315284_10013693 |
| 1056 | Chlorogloeopsis fritschii PCC 6912 sequence18, whole genome shotgun sequence |
| 1057 | Nostoc sp. ATCC 53789 scaffold95.1, whole genome shotgun sequence |
| 1058 | Cyanobacteria bacterium J083 k99_1223732, whole genome shotgun sequence |
| 1059 | Acaryochloris sp. CCMEE 5410 contig00452, whole genome shotgun sequence |
| 1060 | Brasilonema bromeliae SPC951 NODE_2093_length_16008_cov_5.537140, whole |
| | genome shotgun sequence |
| 1061 | Microcystis aeruginosa NIES-3804 DNA, sequence020, whole genome shotgun |
| | sequence |
| 1062 | Moorea sp. SIO4E2 4E2_NODE_211, whole genome shotgun sequence |
| 1063 | Candidatus Poribacteria bacterium isolate SB0668_bin_40 |
| | NODE_6291_length_5861_cov_5.669480, whole genome shotgun sequence |
| 1064 | Dolichospermum sp. UHCC 0406 scaffold19_cov315, whole genome shotgun |
| | sequence |
| 1065 | Arthrospira platensis NIES-46 DNA, sequence091, whole genome shotgun sequence |
| 1066 | Candidatus Poribacteria bacterium isolate SB0672_bin_19 |
| | NODE_4287_length_5454_cov_6.569735, whole genome shotgun sequence |
| 1067 | Dolichospermum sp. UHCC 0259 scaffold237_cov0, whole genome shotgun |
| | sequence |
| 1068 | Dolichospermum sp. UHCC 0352 scaffold173_cov0, whole genome shotgun |
| | sequence |
| 1069 | Calothrix sp. HK-06 NIES-2101_Scaffold_160, whole genome shotgun sequence |
| 1070 | fermentation metagenome genome assembly, contig: |
| | NODE_71_length_108958_cov_61.855514, whole genome shotgun sequence |
| 1071 | Moorea sp. SIO3B2 3B2_NODE_204, whole genome shotgun sequence |
| 1072 | Ga0315284_10116105 |
| 1073 | Ga0373628_0002741 |
| 1074 | Nostoc sp. PCC 7120 = FACHB-418 contig49, whole genome shotgun sequence |
| 1075 | Mastigocoleus testarum BC008 Contig-75, whole genome shotgun sequence |
| 1076 | Prochlorothrix hollandica PCC 9006 = CALU 1027 strain PCC 9006 |
| | Pro9006DRAFT_Contig10.1_C42, whole genome shotgun sequence |
| 1077 | bioreactor sludge metagenome genome assembly, contig: |
| | NODE_19_length_148633_cov_33.775869, whole genome shotgun sequence |
| 1078 | Ga0315280_10003927 |
| 1079 | TPA_asm: Microcoleaceae bacterium UBA10368 contig_309, whole genome |
| | shotgun sequence |
| 1080 | Cyanobacteria bacterium QH_7_48_89 qh_7_scaffold_1052, whole genome shotgun |
| | sequence |
| 1081 | Coleofasciculus chthonoplastes PCC 7420 ctg_1103659003681, whole genome |
| | shotgun sequence |
| 1082 | Microcystis aeruginosa NIES-44 DNA, contig: contig1_21, strain: NIES-44, whole |
| | genome shotgun sequence |
| 1083 | Calothrix elsteri CCALA 953 scaffold16, whole genome shotgun sequence |
| 1084 | Acidithiobacillus thiooxidans strain CLST scaffold00011, whole genome shotgun |
| | sequence |
| 1085 | Ga0315296_10000501 |
| 1086 | Moorea producens 3L ctg45847, whole genome shotgun sequence |
| 1087 | Oscillatoriales cyanobacterium isolate PH2015_14S_45_132 |
| | PH2015_14S_scaffold_482, whole genome shotgun sequence |
| 1088 | Ga0315280_10070155 |
| 1089 | Candidatus Poribacteria bacterium isolate PCPOR1 Ga0206387_1076, whole |
| | genome shotgun sequence |
| 1090 | Candidatus Poribacteria bacterium isolate PCPOR4 Ga0207198_1143, whole |
| | genome shotgun sequence |
| 1091 | Candidatus Poribacteria bacterium isolate Plut_88864 |
| | filtered_Plut_88864_genomic_contig_5604, whole genome shotgun sequence |
| 1092 | Scytonema sp. HK-05 NIES-2130_Scaffold_4, whole genome shotgun sequence |
| 1093 | TPA_asm: Synechococcus sp. isolate SpSt-285 Ga0101945_1002032, whole genome |
| | shotgun sequence |
| 1094 | Coleofasciculus chthonoplastes PCC 7420 ctg_1103659003671, whole genome |
| | shotgun sequence |
| 1095 | Ga0373630_0000877 |
| 1096 | Cyanobacteria bacterium QS_9_48_30 qs_9_scaffold_2173, whole genome shotgun |
| | sequence |
| 1097 | TPA_asm: Syntrophomonas sp. UBA4807 UBA4807_contig_171181, whole genome |
| | shotgun sequence |
| 1098 | Microcystis aeruginosa NIES-44 DNA, contig: contig2_19, strain: NIES-44, whole |
| | genome shotgun sequence |
| 1099 | Microcystis panniformis Mp_MB_F_20051200_S9D S9D_312, whole genome |
| | shotgun sequence |
| 1100 | Ga0315295_10006545 |
| 1101 | metagenomes genome assembly, contig: NODE_4045_length_4818_cov_2.671426, |
| | whole genome shotgun sequence |
| 1102 | Oscillatoriales cyanobacterium isolate PH2015_09S_45 247 |
| | PH2015_09S_scaffold_420, whole genome shotgun sequence |
| 1103 | Moorea sp. SIO4A1 4A1_NODE_34, whole genome shotgun sequence |
| 1104 | Oscillatoriales cyanobacterium isolate PH2015_07U_46_236 |
| | PH2015_07U_scaffold_109, whole genome shotgun sequence |
| 1105 | Ga0373625_0000114 |
| 1106 | Microcystis aeruginosa F13-15 Ga0187998_10047, whole genome shotgun sequence |
| 1107 | Moorea sp. SIO4E2 4E2_NODE_6, whole genome shotgun sequence |
| 1108 | Coleofasciculus sp. Co-bin14 k141_4776899_length_6599_cov_7.0000, whole |
| | genome shotgun sequence |
| 1109 | Ga0315279_10074524 |
| 1110 | TPA_asm: Richelia sp. UBA3957 UBA3957_contig_489, whole genome shotgun |
| | sequence |
| 1111 | Ga0315294_10043458 |
| 1112 | Pleurocapsa sp. PCC 7319 genomic scaffold Pleur7319scaffold_3, whole genome |
| | shotgun sequence |
| 1113 | Oscillatoriales cyanobacterium isolate PH2015_06S_46_2585 |
| | PH2015_06S_scaffold_13083, whole genome shotgun sequence |
| 1114 | Nostoc sp. 106C 19_117334_26.4148_51_117334_0.395332981062608, whole |
| | genome shotgun sequence |
| 1115 | Cyanobacteria bacterium QS_6_48_18 qs_6_scaffold_1172, whole genome shotgun |
| | sequence |
| 1116 | Moorea sp. SIO3E8 3E8_NODE_21, whole genome shotgun sequence |
| 1117 | Microcystis wesenbergii Mw_QC_B_20070930_S4 S4_116, whole genome shotgun |
| | sequence |
| 1118 | Oscillatoria sp. isolate S11.Bin022 Scaffold_23, whole genome shotgun sequence |
| 1119 | Scytonema sp. UIC 10036 c00129_NODE_12 . . . , whole genome shotgun sequence |
| 1120 | Ga0373626_0013195 |
| 1121 | Methanoculleus sp. isolate WOFA03 NODE_6082_length_5724_cov_31.431293, |
| | whole genome shotgun sequence |
| 1122 | Gloeocapsa sp. PCC 73106 scaffold_00137, whole genome shotgun sequence |
| 1123 | Lyngbya sp. PCC 8106 1099428180525, whole genome shotgun sequence |
| 1124 | Chamaesiphon polymorphus CCALA 037 |
| | NODE_187_length_14934_cov_6.09504_ID_66389, whole genome shotgun |
| | sequence |
| 1125 | Hydrococcus sp. RU_2_2 NODE_50_length_83323_cov_3.681956, whole genome |
| | shotgun sequence |
| 1126 | Microcystis aeruginosa Ma_SC_T_19800800_S464 S464_237, whole genome |
| | shotgun sequence |
| 1127 | fermentation metagenome genome assembly, contig: |
| | NODE_8578_length_5580_cov_8.227511, whole genome shotgun sequence |
| 1128 | Oscillatoriales cyanobacterium isolate PH2015_07D_46_1245 |
| | PH2015_07D_scaffold_1762, whole genome shotgun sequence |
| 1129 | Richelia sp. SL_2_1 NODE_95_length_50065_cov_8.922143, whole genome |
| | shotgun sequence |
| 1130 | Moorea sp. SIO3I7 3I7_NODE_589, whole genome shotgun sequence |
| 1131 | Scytonema sp. HK-05 NIES-2130_Scaffold_20, whole genome shotgun sequence |
| 1132 | Microcystis aeruginosa 9701 WGS project CAIQ01000000 data, contig |
| | AAI_K_2204_21, whole genome shotgun sequence |
| 1133 | Ga0315273_10121282 |
| 1134 | Moorea sp. SIO4A3 4A3_NODE_797, whole genome shotgun sequence |
| 1135 | Desulfotomaculum australicum DSM 11792 genome assembly, contig: |
| | EJ60DRAFT_scaffold00036.36, whole genome shotgun sequence |
| 1136 | Ga0373630_0013875 |
| 1137 | Mastigocoleus testarum BC008 YY1DRAFT_scaffold_68.69_C, whole genome |
| | shotgun sequence |
| 1138 | sediment metagenome genome assembly, contig: |
| | NODE_72_length_3716_cov_2.895930, whole genome shotgun sequence |
| 1139 | Ga0315285_10058279 |
| 1140 | Aphanothece sacrum FPU3 DNA, contig: ASFPU3_contig081, whole genome |
| | shotgun sequence |
| 1141 | Oscillatoriales cyanobacterium isolate PH2015_04D_45_965 |
| | PH2015_04D_scaffold_1308, whole genome shotgun sequence |
| 1142 | Archaeon SCG-AAA382B04 isolate SCG-AAA382B04 AAA382B04_1, whole |
| | genome shotgun sequence |
| 1143 | Geitlerinema sp. PCC 7105 Gei7105DRAFT_GPC.5_C29, whole genome shotgun |
| | sequence |
| 1144 | Candidatus Poribacteria bacterium isolate PCPOR1 Ga0206387_1124, whole |
| | genome shotgun sequence |
| 1145 | Candidatus Poribacteria bacterium isolate SB0672_bin_19 |
| | NODE_3818_length_5909_cov_5.206355, whole genome shotgun sequence |
| 1146 | Anabaena lutea FACHB-196 contig37, whole genome shotgun sequence |
| 1147 | Microcystis aeruginosa NIES-4264 DNA, contig_41, whole genome shotgun |
| | sequence |
| 1148 | Ardenticatenales bacterium isolate MGR_bin47 SD2897-2912_k127_572466, whole |
| | genome shotgun sequence |
| 1149 | Ga0315294_10088530 |
| 1150 | Brasilonema sp. UFV-L1 scaffold102_cov13, whole genome shotgun sequence |
| 1151 | Chloroflexi bacterium isolate CF_117 14_0929_09_20cm_scaffold_32114, whole |
| | genome shotgun sequence |
| 1152 | Synechococcus sp. PCC 7335 scf_1103496006892 genomic scaffold, whole genome |
| | shotgun sequence |
| 1153 | [Scytonema hofmanni] UTEX 2349 Tol9009DRAFT_TPD.8_C41, whole genome |
| | shotgun sequence |
| 1154 | Oscillatoriales cyanobacterium isolate PH2015_07D 46 1245 |
| | PH2015_07D_scaffold_1861, whole genome shotgun sequence |
| 1155 | Microcystis flos-aquae FACHB-1344 contig135, whole genome shotgun sequence |
| 1156 | Ga0373626_0004519 |
| 1157 | Crocosphaera watsonii WH 8501 ctg345, whole genome shotgun sequence |
| 1158 | Oscillatoriales cyanobacterium isolate PH2015_01D_44_513 |
| | PH2015_01D_scaffold_4440, whole genome shotgun sequence |
| 1159 | Ga0373629_0013319 |
| 1160 | fermentation metagenome genome assembly, contig: |
| | NODE_6106_length_8625_cov_8.304084, whole genome shotgun sequence |
| 1161 | Candidatus Poribacteria bacterium isolate PCPOR2a Ga0207197_1069, whole |
| | genome shotgun sequence |
| 1162 | Candidatus Poribacteria bacterium isolate AGPOR5 scf7180000041070, whole |
| | genome shotgun sequence |
| 1163 | Ga0373625_0098307 |
| 1164 | Okeania sp. KiyG1 DNA, sequence085, whole genome shotgun sequence |
| 1165 | Crocosphaera watsonii WH 0005 WGS project CAQL00000000 data, contig 01056, |
| | whole genome shotgun sequence |
| 1166 | Scytonema sp. UIC 10036 c00414_NODE_41 . . . , whole genome shotgun sequence |
| 1167 | Candidatus Gracilibacteria bacterium isolate SU_1_2 |
| | NODE_2830_length_5569_cov_3.759096, whole genome shotgun sequence |
| 1168 | Planktothrix prolifica NIVA-CYA 540 contig00042, whole genome shotgun |
| | sequence |
| 1169 | Crocosphaera sp. isolate DT_26 DT-Crocosphaera-1_scaffold_601, whole genome |
| | shotgun sequence |
| 1170 | Candidatus Poribacteria bacterium isolate PCPOR4 Ga0207198_1028, whole |
| | genome shotgun sequence |
| 1171 | Chlorogloeopsis fritschii PCC 6912 contig00059, whole genome shotgun sequence |
| 1172 | Moorea sp. SIO4G2 4G2_NODE_3083, whole genome shotgun sequence |
| 1173 | Cyanobacteria bacterium QH_6_48_35 qh_6_scaffold_2052, whole genome shotgun |
| | sequence |
| 1174 | TPA_asm: Cyanobacteria bacterium UBA8553 contig_1147, whole genome shotgun |
| | sequence |
| 1175 | Microcystis aeruginosa Ma_SC_T_19800800_S464 S464_89, whole genome |
| | shotgun sequence |
| 1176 | wastewater metagenome genome assembly, contig: |
| | NODE_10_length_147907_cov_31.462266, whole genome shotgun sequence |
| 1177 | TPA_asm: Cyanobacteria bacterium UBA8803 contig_185, whole genome shotgun |
| | sequence |
| 1178 | Scytonema sp. CRU_2_7 NODE_5865_length_4978_cov_2.76335, whole genome |
| | shotgun sequence |
| 1179 | Moorea sp. SIO3H5 3H5_NODE_395, whole genome shotgun sequence |
| 1180 | Ga0315298_1001267 |
| 1181 | Tolypothrix sp. T3-bin4 k141_222205_length_2371_cov_6.0000, whole genome |
| | shotgun sequence |
| 1182 | Candidatus Poribacteria bacterium isolate SB0668_bin_40 |
| | NODE_1215_length_20491_cov_8.127373, whole genome shotgun sequence |
| 1183 | Ga0315284_10084224 |
| 1184 | Ga0315280_10000556 |
| 1185 | Chlorogloeopsis fritschii PCC 6912 sequence18, whole genome shotgun sequence |
| 1186 | Microcystis aeruginosa NIES-2522 DNA, contig_16, whole genome shotgun |
| | sequence |
| 1187 | Nitrosococcus oceani strain AFC132 contig_24, whole genome shotgun sequence |
| 1188 | Microcystis flos-aquae Mf_QC_C_20070823_S10 S10_205, whole genome shotgun |
| | sequence |
| 1189 | Lyngbya majuscula 3L genomic scaffold scf52022, whole genome shotgun sequence |
| 1190 | Microcoleus sp. FACHB-84 contig13, whole genome shotgun sequence |
| 1191 | Ga0307928_10038209 |
| 1192 | metagenome genome assembly, contig: NODE_111_length_94772_cov_5.504176, |
| | whole genome shotgun sequence |
| 1193 | Planktothrix agardhii CCAP 1459/11A NIES-905 DNA, sequence019, whole |
| | genome shotgun sequence |
| 1194 | uncultured Oscillatoriales cyanobacterium isolate KTt_bin-1839 genome assembly, |
| | contig: bin-1839: 147/277, whole genome shotgun sequence |
| 1195 | Cyanobacteria bacterium QH_3_48_40 qh_3_scaffold_563, whole genome shotgun |
| | sequence |
| 1196 | Arthrospira platensis NIES-46 DNA, sequence003, whole genome shotgun sequence |
| 1197 | Crocosphaera watsonii WH 0401 WGS project CAQM00000000 data, contig 01078, |
| | whole genome shotgun sequence |
| 1198 | Crocosphaera watsonii WH 8502 WGS project CAQK00000000 data, contig 00002, |
| | whole genome shotgun sequence |
| 1199 | Microcoleus sp. Co-bin12 k141_6726532_length_3994_cov_14.2126, whole genome |
| | shotgun sequence |
| 1200 | Ga0373630_0001176 |
| 1201 | uncultured Microcoleus sp. isolate AVDCRST_MAG84 genome assembly, contig: |
| | NODE2305, whole genome shotgun sequence |
| 1202 | Candidatus Poribacteria bacterium isolate PCPOR4 Ga0207198_1029, whole |
| | genome shotgun sequence |
| 1203 | Sponge metagenome NODE_9472_length_8435_cov_19.5502_ID_18943, whole |
| | genome shotgun sequence |
| 1204 | Scytonema sp. UIC 10036 c00181_NODE_18 . . . , whole genome shotgun sequence |
| 1205 | Ga0315280_10000403 |
| 1206 | Brasilonema bromeliae SPC951 NODE_3533_length_9450_cov_4.016285, whole |
| | genome shotgun sequence |
| 1207 | Richelia sp. CSU_2_1 NODE_496_length_25657_cov_10.0371, whole genome |
| | shotgun sequence |
| 1208 | uncultured cyanobacterium isolate B12_bin-1940 genome assembly, contig: bin- |
| | 1940: 1549/1810, whole genome shotgun sequence |
| 1209 | Trichocoleus sp. FACHB-832 contig52, whole genome shotgun sequence |
| 1210 | Ga0315285_10002437 |
| 1211 | Hapalosiphonaceae cyanobacterium JJU2 Scaffold_54, whole genome shotgun |
| | sequence |
| 1212 | Oscillatoriales cyanobacterium isolate PH2015_08D_46_1646 |
| | PH2015_08D_scaffold_2263, whole genome shotgun sequence |
| 1213 | Microcystis aeruginosa NIES-87 DNA, sequence016, whole genome shotgun |
| | sequence |
| 1214 | Ga0307928_10013595 |
| 1215 | Scytonema sp. HK-05 NIES-2130_Scaffold_36, whole genome shotgun sequence |
| 1216 | uncultured archaeon isolate LFW-68_2 genome assembly, contig: LFW682_39, |
| | whole genome shotgun sequence |
| 1217 | fermentation metagenome genome assembly, contig: |
| | NODE_2539_length_14463_cov 6.300250, whole genome shotgun sequence |
| 1218 | Tychonema bourrellyi FEM_GT703 scaffold38_size30849, whole genome shotgun |
| | sequence |
| 1219 | Microcystis aeruginosa BLCCF108 NODE_334_length_13058_cov_116.817888, |
| | whole genome shotgun sequence |
| 1220 | Ga0315277_10011256 |
| 1221 | coral metagenome genome assembly, contig: |
| | NODE_159_length_19067_cov 12.431648, whole genome shotgun sequence |
| 1222 | Ktedonobacter sp. 13_1_20CM_3_54_15 13_1_20cm_3_scaffold_1163, whole |
| | genome shotgun sequence |
| 1223 | Ga0315273_10078206 |
| 1224 | Scytonema sp. HK-05 NIES-2130_Scaffold_10, whole genome shotgun sequence |
| 1225 | Scytonema sp. HK-05 NIES-2130_Scaffold_125, whole genome shotgun sequence |
| 1226 | fermentation metagenome genome assembly, contig: |
| | NODE_4309_length_10165_cov_34.872404, whole genome shotgun sequence |
| 1227 | Cyanobacteria bacterium RU_5_0 NODE_1542_length_16773_cov_21.027214, |
| | whole genome shotgun sequence |
| 1228 | TPA_asm: Richelia sp. UBA3958 UBA3958_contig_482, whole genome shotgun |
| | sequence |
| 1229 | Scytonema sp. UIC 10036 c00130_NODE_13 . . . , whole genome shotgun sequence |
| 1230 | Candidatus Chloroploca asiatica strain B7-9 |
| | NODE_14_length_96467_cov_78.3642_ID_198493, whole genome shotgun |
| | sequence |
| 1231 | Moorea sp. SIO3A2 3A2_NODE_46, whole genome shotgun sequence |
| 1232 | Pleurocapsa sp. CCALA 161 NODE_10_length_78268_cov_10.6832_ID_51584, |
| | whole genome shotgun sequence |
| 1233 | cyanobacterium TDX16 genomic scaffold Scaffold94, whole genome shotgun |
| | sequence |
| 1234 | Ga0315275_10032158 |
| 1235 | Ga0315295_10005530 |
| 1236 | Ga0373625_0144508 |
| 1237 | Moorea sp. SIO3I6 3I6_NODE_987, whole genome shotgun sequence |
| 1238 | Ga0373627_0035378 |
| 1239 | marine metagenome genome assembly, contig: |
| | NODE_5726_length_4256_cov_3.579325, whole genome shotgun sequence |
| 1240 | Ga0373629_0019898 |
| 1241 | Microcystis aeruginosa LG13-12 Ga0188023_10518, whole genome shotgun |
| | sequence |
| 1242 | Calothrix sp. HK-06 NIES-2101_Scaffold 160, whole genome shotgun sequence |
| 1243 | Ga0373627_0026263 |
| 1244 | Scytonema sp. UIC 10036 c00181_NODE_18 . . . , whole genome shotgun sequence |
| 1245 | uncultured Methanomicrobiales archaeon isolate AM-sed-core3-D1_bin-531 genome |
| | assembly, contig: bin-531: 147/408, whole genome shotgun sequence |
| 1246 | Microcystis panniformis Mp_MB_F_20051200_S9 S9_160, whole genome shotgun |
| | sequence |
| 1247 | Activated sludge metagenome, whole genome shotgun sequence |
| 1248 | Ga0373629_0001148 |
| 1249 | Ga0315280_10050744 |
| 1250 | TPA_asm: Blastocatellia bacterium isolate SpSt-338 Ga0073934_10003832, whole |
| | genome shotgun sequence |
| 1251 | Leptolyngbya valderiana BDU 20041 contig00137, whole genome shotgun sequence |
| 1252 | Anabaena catenula FACHB-362 contig6, whole genome shotgun sequence |
| 1253 | Kamptonema sp. SIO1D9 1D9_NODE_9, whole genome shotgun sequence |
| 1254 | Ga0373630_0100330 |
| 1255 | Ga0315285_10002913 |
| 1256 | Candidatus Poribacteria bacterium bin44 |
| | NODE_523_length_80671_cov_19.5214_ID_1045, whole genome shotgun sequence |
| 1257 | Sulfobacillus benefaciens isolate AMDSBA4 AMDSBA4_120, whole genome |
| | shotgun sequence |
| 1258 | Oscillatoriales cyanobacterium isolate PH2015_02D_45_1038 |
| | PH2015_02D_scaffold_875, whole genome shotgun sequence |
| 1259 | Oscillatoriales cyanobacterium isolate PH2015_08D_46_1646 |
| | PH2015_08D_scaffold_5893, whole genome shotgun sequence |
| 1260 | Terrestrial metagenome MPI_scaffold_4171, whole genome shotgun sequence |
| 1261 | Candidatus Poribacteria bacterium isolate SB0675_bin_22 |
| | NODE_1577_length_16369_cov_11.811818, whole genome shotgun sequence |
| 1262 | Microcystis aeruginosa PCC 9717 genomic scaffold, AAI_B_2196_scaffold69, |
| | whole genome shotgun sequence |
| 1263 | Oscillatoriales cyanobacterium isolate PH2015_13U_45_97 |
| | PH2015_13U_scaffold_350, whole genome shotgun sequence |
| 1264 | Ga0315284_10000891 |
| 1265 | Candidatus Methanoculleus thermohydrogenotrophicum isolate DTU006 |
| | scaffold1316, whole genome shotgun sequence |
| 1266 | Coleofasciculus sp. Co-bin14 k141_2884914_length_2356_cov_128.0000, whole |
| | genome shotgun sequence |
| 1267 | Ga0315280_10012508 |
| 1268 | Oscillatoriales cyanobacterium isolate PH2015_12U_45_315 |
| | PH2015_12U_scaffold_23, whole genome shotgun sequence |
| 1269 | Moorea sp. SIO2C4 2C4_NODE_1155, whole genome shotgun sequence |
| 1270 | Chlorogloea sp. CCALA 695 NODE_68_length_40204_cov_6.81018_ID_66494, |
| | whole genome shotgun sequence |
| 1271 | Microcystis flos-aquae Ma_QC_C_20070823_S18D S18D_53, whole genome |
| | shotgun sequence |
| 1272 | Brasilonema sp. UFV-L1 scaffold148_cov13, whole genome shotgun sequence |
| 1273 | Cyanobacteria bacterium J055 k99_1058335, whole genome shotgun sequence |
| 1274 | Candidatus Poribacteria bacterium isolate Plut_88864 |
| | filtered_Plut_88864_genomic_contig_44193, whole genome shotgun sequence |
| 1275 | Kouleothrix aurantiaca strain COM-B contig_39, whole genome shotgun sequence |
| 1276 | Desulfotomaculum australicum DSM 11792 genome assembly, contig: |
| | EJ60DRAFT_scaffold00036.36, whole genome shotgun sequence |
| 1277 | Ga0373626_0073864 |
| 1278 | Ga0373629_0071313 |
| 1279 | Ga0315279_10001675 |
| 1280 | Crocosphaera watsonii WH0402 WGS project CAQN00000000 data, contig 01993, |
| | whole genome shotgun sequence |
| 1281 | Microcystis aeruginosa NIES-2522 DNA, contig_18, whole genome shotgun |
| | sequence |
| 1282 | Ardenticatenales bacterium isolate MGR_bin47 SD2897-2912_k127_6618261, |
| | whole genome shotgun sequence |
| 1283 | wastewater metagenome genome assembly, contig: |
| | NODE_1502_length_9081_cov_7.120097, whole genome shotgun sequence |
| 1284 | Merismopedia glauca CCAP 1448/3 |
| | NODE_172_length_31568_cov_6.44124_ID_38037, whole genome shotgun |
| | sequence |
| 1285 | Oscillatoriales cyanobacterium isolate PH2015_10S_46_199 |
| | PH2015_10S_scaffold_548, whole genome shotgun sequence |
| 1286 | Limnoraphis robusta CS-951 contig188, whole genome shotgun sequence |
| 1287 | Scytonema hofmannii FACHB-248 contig8, whole genome shotgun sequence |
| 1288 | Ga0315280_10043499 |
| 1289 | Ga0315280_10014824 |
| 1290 | Candidatus Poribacteria bacterium isolate Plut_88885 |
| | unfiltered_Plut_88885_genomic_contig_871155, whole genome shotgun sequence |
| 1291 | Oscillatoriales cyanobacterium isolate PH2015_08D_45_74 |
| | PH2015_08D_scaffold_552, whole genome shotgun sequence |
| 1292 | Pleurocapsa sp. PCC 7319 Pleur7319scaffold_3_Cont9, whole genome shotgun |
| | sequence |
| 1293 | Ardenticatenales bacterium isolate MGR_bin47 SD2897-2912_k127_572466, whole |
| | genome shotgun sequence |
| 1294 | Symploca sp. SIO3C6 3C6_NODE_19, whole genome shotgun sequence |
| 1295 | Planktothrix agardhii NIVA-CYA 34 contig00003, whole genome shotgun sequence |
| 1296 | uncultured cyanobacterium isolate B12_bin-0982 genome assembly, contig: bin- |
| | 0982: 115/268, whole genome shotgun sequence |
| 1297 | TPA asm: Oscillatoriales bacterium UBA8482 contig_54582, whole genome |
| | shotgun sequence |
| 1298 | Chlorogloeopsis fritschii PCC 6912 contig00060, whole genome shotgun sequence |
| 1299 | Ga0315294_10112621 |
| 1300 | Cyanobacteria bacterium QH_10_48_56 qh_10_scaffold_929, whole genome |
| | shotgun sequence |
| 1301 | Crocosphaera watsonii WH 8501 ctg345, whole genome shotgun sequence |
| 1302 | Candidatus Poribacteria bacterium isolate SB0675_bin_22 |
| | NODE_1031_length_22403_cov_12.518346, whole genome shotgun sequence |
| 1303 | TPA_asm: Microcoleaceae bacterium UBA9251 contig_918, whole genome shotgun |
| | sequence |
| 1304 | TPA_asm: Richelia sp. UBA3308 UBA3308_contig_2520, whole genome shotgun |
| | sequence |
| 1305 | Scytonema sp. RU_4_4 NODE_486_length_31581_cov_12.788962, whole genome |
| | shotgun sequence |
| 1306 | Desulfovirgula thermocuniculi DSM 16036 G454DRAFT_scaffold00006.6_C, whole |
| | genome shotgun sequence |
| 1307 | Ardenticatenales bacterium isolate MGR_bin47 SD2897-2912_k127_4223749, |
| | whole genome shotgun sequence |
| 1308 | Microcystis aeruginosa Ma_OC_H_19870700_S124 S124_61, whole genome |
| | shotgun sequence |
| 1309 | Oscillatoriales cyanobacterium isolate PH2015_01D_44_513 |
| | PH2015_01D_scaffold_6706, whole genome shotgun sequence |
| 1310 | Ga0315284_10000823 |
| 1311 | TPA_asm: Cyanobacteria bacterium UBA11369 contig_453, whole genome shotgun |
| | sequence |
| 1312 | uncultured Opitutae bacterium isolate MJ-time_bin-6334 genome assembly, contig: |
| | bin-6334: 077/164, whole genome shotgun sequence |
| 1313 | Ga0373626_0107639 |
| 1314 | Ga0307928_10000796 |
| 1315 | Mastigocoleus testarum BC008 Contig-75, whole genome shotgun sequence |
| 1316 | Microbial mat metagenome MIS-Ph1_c201, whole genome shotgun sequence |
| 1317 | Ga0315294_10113088 |
| 1318 | Microcystis aeruginosa 11-30S32 DNA, 11-30S32contig365, whole genome shotgun |
| | sequence |
| 1319 | Sulfobacillus sp. DSM 109850 NODE_109_length_10971_cov_117.928952, whole |
| | genome shotgun sequence |
| 1320 | Ga0315273_10035469 |
| 1321 | Chloroflexi bacterium isolate B30_G2 B30_Guay2_scaffold_03813, whole genome |
| | shotgun sequence |
| 1322 | Scytonema sp. UIC 10036 c00181_NODE_18 . . . , whole genome shotgun sequence |
| 1323 | TPA_asm: Cyanobacteria bacterium UBA11368 contig_1385, whole genome |
| | shotgun sequence |
| 1324 | Microcystis aeruginosa Ma_SC_T_19800800_S464 S464_237, whole genome |
| | shotgun sequence |
| 1325 | Coleofasciculus chthonoplastes PCC 7420 ctg_1103659003736, whole genome |
| | shotgun sequence |
| 1326 | uncultured cyanobacterium isolate B12_bin-0982 genome assembly, contig: bin- |
| | 0982: 254/268, whole genome shotgun sequence |
| 1327 | Oscillatoriales cyanobacterium isolate PH2015_06S_54_30 |
| | PH2015_06S_scaffold_4063, whole genome shotgun sequence |
| 1328 | Hydrococcus sp. RU_2_2 NODE_1097_length_20307_cov_3.458276, whole |
| | genome shotgun sequence |
| 1329 | Brasilonema bromeliae SPC951 NODE_2367_length_14264_cov_4.685270, whole |
| | genome shotgun sequence |
| 1330 | Ga0315284_10131710 |
| 1331 | Moorea sp. SIO4G2 4G2_NODE_3083, whole genome shotgun sequence |
| 1332 | Moorea sp. SIO2I5 2I5_NODE_1588, whole genome shotgun sequence |
| 1333 | activated sludge metagenome genome assembly, contig: |
| | NODE_7171_length_4072_cov_711.029, whole genome shotgun sequence |
| 1334 | uncultured Microcoleus sp. isolate AVDCRST_MAG84 genome assembly, contig: |
| | NODE1784, whole genome shotgun sequence |
| 1335 | Ga0307928_10010650 |
| 1336 | Scytonema sp. UIC 10036 c00129_NODE_12 . . . , whole genome shotgun sequence |
| 1337 | Planktothrix prolifica NIVA-CYA 98 contig00006, whole genome shotgun sequence |
| 1338 | Synechococcus sp. PCC 7335 ctg_1103496006875, whole genome shotgun sequence |
| 1339 | Ga0315285_10061643 |
| 1340 | Ga0373625_0099120 |
| 1341 | Coleofasciculaceae cyanobacterium RL_1_1 |
| | NODE_927_length_11201_cov_3.089760, whole genome shotgun sequence |
| 1342 | Crocosphaera watsonii WH 0005 WGS project CAQL00000000 data, contig 00096, |
| | whole genome shotgun sequence |
| 1343 | Ga0315285_10001508 |
| 1344 | Microcoleus chthonoplastes PCC 7420 scf_1103659003800 genomic scaffold, whole |
| | genome shotgun sequence |
| 1345 | Oscillatoriales cyanobacterium isolate PH2015_02D_45_1038 |
| | PH2015_02D_scaffold_702, whole genome shotgun sequence |
| 1346 | TPA_asm: Cyanobacteria bacterium UBA11372 contig_575, whole genome shotgun |
| | sequence |
| 1347 | TPA_asm: Pseudanabaena sp. UBA1465 UBA1465_contig_2117, whole genome |
| | shotgun sequence |
| 1348 | Candidatus Poribacteria bacterium isolate Plut_88885 |
| | unfiltered_Plut_88885_genomic_contig_552830, whole genome shotgun sequence |
| 1349 | Ktedonobacter sp. 13_1_20CM_4_53_11 13_1_20cm_4_scaffold_1247, whole |
| | genome shotgun sequence |
| 1350 | Ga0373628_0143129 |
| 1351 | Ga0307928_10017022 |
| 1352 | Scytonema sp. UIC 10036 c00161_NODE_16 . . . , whole genome shotgun sequence |
| 1353 | coral metagenome genome assembly, contig: |
| | NODE_189_length_14262_cov_7.383786, whole genome shotgun sequence |
| 1354 | cyanobacterium TDX16 genomic scaffold Scaffold94, whole genome shotgun |
| | sequence |
| 1355 | Ga0373626_0068106 |
| 1356 | Symploca sp. SIO1B1 1B1_NODE_765, whole genome shotgun sequence |
| 1357 | Candidatus Poribacteria bacterium isolate PNGco_C_binSS2 scaffold_249, whole |
| | genome shotgun sequence |
| 1358 | Ga0315284_10173532 |
| 1359 | Ga0315279_10038201 |
| 1360 | Moorea sp. SIO2I5 2I5_NODE_1588, whole genome shotgun sequence |
| 1361 | Halothece sp. KZN 001 cyano_08_contig_431, whole genome shotgun sequence |
| 1362 | Planktothrix prolifica NIVA-CYA 98 contig00257, whole genome shotgun sequence |
| 1363 | Sediment metagenome Contig_342, whole genome shotgun sequence |
| 1364 | Ga0255812_10495892 |
| 1365 | Candidatus Poribacteria bacterium isolate SB0662_bin_35 |
| | NODE_7525_length_6951_cov_34.020302, whole genome shotgun sequence |
| 1366 | Methanocalculus sp. isolate CSSed165cm_604R1 CSSed16-5cm-4263, whole |
| | genome shotgun sequence |
| 1367 | Planktothrix sp. FACHB-1355 contig495, whole genome shotgun sequence |
| 1368 | TPA_asm: Cyanobacteria bacterium UBA11049 contig_22115, whole genome |
| | shotgun sequence |
| 1369 | uncultured Oscillatoriales cyanobacterium isolate Day2-6_bin-615 genome assembly, |
| | contig: bin-615: 0522/1053, whole genome shotgun sequence |
| 1370 | Leptolyngbya sp. FACHB-239 contig16, whole genome shotgun sequence |
| 1371 | Candidatus Poribacteria bacterium isolate SB0675_bin_22 |
| | NODE_404_length_42574_cov_13.168842, whole genome shotgun sequence |
| 1372 | metagenome genome assembly, contig: NODE_4871_length_9566_cov_272.221203, |
| | whole genome shotgun sequence |
| 1373 | Anabaena sp. FACHB-709 contig44, whole genome shotgun sequence |
| 1374 | Moorea producens 3L ctg45798, whole genome shotgun sequence |
| 1375 | Crocosphaera watsonii WH 8502 WGS project CAQK00000000 data, contig 00461, |
| | whole genome shotgun sequence |
| 1376 | Ga0373628_0000972 |
| 1377 | fermentation metagenome genome assembly, contig: |
| | NODE_1189_length_28905_cov_9.161906, whole genome shotgun sequence |
| 1378 | Oscillatoriales cyanobacterium isolate PH2015_01U_45_14 |
| | PH2015_01U_scaffold_206, whole genome shotgun sequence |
| 1379 | Ga0315284_10133469 |
| 1380 | Ga0373630_0081576 |
| 1381 | Leptolyngbya sp. FACHB-541 contig14, whole genome shotgun sequence |
| 1382 | Microcystis aeruginosa Ma_QC_B_20070730_S2 S2_44, whole genome shotgun |
| | sequence |
| 1383 | Ga0373625_0195404 |
| 1384 | Symploca sp. SIO2C1 2C1_NODE_95, whole genome shotgun sequence |
| 1385 | Gloeocapsa sp. PCC 73106 scaffold_00213, whole genome shotgun sequence |
| 1386 | Microcoleus sp. SM1_3_4 NODE_1886_length_13372_cov_2.009589, whole |
| | genome shotgun sequence |
| 1387 | Planktothrix mougeotii NIVA-CYA 405 contig00127, whole genome shotgun |
| | sequence |
| 1388 | Moorea sp. SIO3G5 3G5_NODE_356, whole genome shotgun sequence |
| 1389 | Tychonema bourrellyi FEM_GT703 scaffold98_size18864, whole genome shotgun |
| | sequence |
| 1390 | TPA_asm: Candidatus Bathyarchaeota archaeon isolate SpSt-1006 |
| | Ga0123519_10026078, whole genome shotgun sequence |
| 1391 | Ga0373625_0005445 |
| 1392 | Candidatus Poribacteria bacterium isolate SB0672_bin_19 |
| | NODE_10240_length_2960_cov_3.738382, whole genome shotgun sequence |
| 1393 | Moorea sp. SIO2B7 2B7_NODE_1079, whole genome shotgun sequence |
| 1394 | Candidatus Poribacteria bacterium bin44 |
| | NODE_1143_length_51058_cov_18.8876_ID_2285, whole genome shotgun |
| | sequence |
| 1395 | Moorea sp. SIO2C4 2C4_NODE_709, whole genome shotgun sequence |
| 1396 | Microcystis sp. T1-4 WGS project CAIP01000000 data, contig AAI_I_2203_23, |
| | whole genome shotgun sequence |
| 1397 | Arthrospira sp. PCC 8005, WGS project CAFN00000000 data, strain PCC 8005, |
| | Contig4958-2130, whole genome shotgun sequence |
| 1398 | Candidatus Bathyarchaeota archaeon BA1 ba1_04, whole genome shotgun sequence |
| 1399 | Okeania sp. SIO3B5 3B5_NODE_236, whole genome shotgun sequence |
| 1400 | Ga0315295_10030336 |
| 1401 | Sponge metagenome NODE_28028_length_3465_cov_3.81096_ID_56055, whole |
| | genome shotgun sequence |
| 1402 | Ga0315296_10008306 |
| 1403 | TPA_asm: Cyanobacteria bacterium UBA12227 contig_617, whole genome shotgun |
| | sequence |
| 1404 | Ga0315295_10030299 |
| 1405 | Crocosphaera watsonii WH 0003 Contig00520, whole genome shotgun sequence |
| 1406 | Kamptonema sp. SIO4C4 4C4_NODE_1097, whole genome shotgun sequence |
| 1407 | freshwater metagenome genome assembly, contig: |
| | NODE_87_length_5282_cov_5.874685, whole genome shotgun sequence |
| 1408 | Moorea sp. SIO3G5 3G5_NODE_1620, whole genome shotgun sequence |
| 1409 | Ga0373629_0021606 |
| 1410 | Scytonema sp. HK-05 NIES-2130_Scaffold_72, whole genome shotgun sequence |
| 1411 | Microcystis panniformis Mp_MB_F_20051200_S6D S6D_8, whole genome shotgun |
| | sequence |
| 1412 | Ga0315280_10050637 |
| 1413 | Candidatus Poribacteria bacterium isolate SB0669_bin_10 |
| | NODE_4394_length_4900_cov_7.727761, whole genome shotgun sequence |
| 1414 | Ga0315280_10000252 |
| 1415 | Microcoleus sp. Co-bin12 k141_16798132_length_1964_cov_17.0000, whole |
| | genome shotgun sequence |
| 1416 | Ga0315284_10166547 |
| 1417 | Ga0315284_10098300 |
| 1418 | Moorea sp. SIO3I7 3I7_NODE_1751, whole genome shotgun sequence |
| 1419 | Okeania sp. SIO3B5 3B5_NODE_379, whole genome shotgun sequence |
| 1420 | Microcystis flos-aquae Mf_QC_C_20070823_S20 S20_49, whole genome shotgun |
| | sequence |
| 1421 | Limnospira fusiformis KN contig55, whole genome shotgun sequence |
| 1422 | Dolichospermum sp. UHCC 0299 scaffold121_cov0, whole genome shotgun |
| | sequence |
| 1423 | Crocosphaera watsonii WH 0003 Contig00520, whole genome shotgun sequence |
| 1424 | Candidatus Poribacteria bacterium isolate SB0663_bin_6 |
| | NODE_1869_length_7015_cov_7.269828, whole genome shotgun sequence |
| 1425 | Ga0373629_0000692 |
| 1426 | Calothrix elsteri CCALA 953 scaffold16, whole genome shotgun sequence |
| 1427 | uncultured cyanobacterium isolate A5_bin-0177 genome assembly, contig: bin- |
| | 0177: 296/358, whole genome shotgun sequence |
| 1428 | Anabaena catenula FACHB-362 contig23, whole genome shotgun sequence |
| 1429 | Ga0315284_10114303 |
| 1430 | Candidatus Poribacteria bacterium isolate PCPOR1 Ga0206387_1124, whole |
| | genome shotgun sequence |
| 1431 | Ga0315294_10032729 |
| 1432 | Ga0315275_10057549 |
| 1433 | Scytonema hofmannii FACHB-248 contig68, whole genome shotgun sequence |
| 1434 | Oscillatoriales cyanobacterium isolate PH2015_03D_45_235 |
| | PH2015_03D_scaffold_616, whole genome shotgun sequence |
| 1435 | wastewater metagenome genome assembly, contig: |
| | NODE_787_length_9569_cov_7.467101, whole genome shotgun sequence |
| 1436 | Kamptonema sp. SIO4C4 4C4_NODE_599, whole genome shotgun sequence |
| 1437 | Cyanobacteria bacterium CRU_2_1 NODE_326_length_28779_cov_5.45215, whole |
| | genome shotgun sequence |
| 1438 | Scytonema sp. UIC 10036 c00414_NODE_41 . . . , whole genome shotgun sequence |
| 1439 | Ga0373625_0072956 |
| 1440 | Candidatus Poribacteria bacterium isolate SB0669_bin_10 |
| | NODE_1037_length_12836_cov_8.716219, whole genome shotgun sequence |
| 1441 | TPA_asm: Cyanobacteria bacterium UBA8543 contig_1385, whole genome shotgun |
| | sequence |
| 1442 | Okeania sp. SIO3B5 3B5_NODE_379, whole genome shotgun sequence |
| 1443 | Candidatus Poribacteria bacterium isolate SB0668_bin_36 |
| | NODE_7567_length_5025_cov_7.988531, whole genome shotgun sequence |
| 1444 | wastewater metagenome genome assembly, contig: |
| | NODE_806_length_13733_cov_6.061851, whole genome shotgun sequence |
| 1445 | Desulfitobacterium chlororespirans DSM 11544 genome assembly, contig: |
| | EJ42DRAFT_scaffold00015.15, whole genome shotgun sequence |
| 1446 | Planktothrix prolifica NIVA-CYA 540 genomic scaffold scaffold00001, whole |
| | genome shotgun sequence |
| 1447 | Microcystis aeruginosa 9809 WGS project CAIO01000000 data, contig |
| | AAI_H_2202_526, whole genome shotgun sequence |
| 1448 | Desulfurispora thermophila DSM 16022 B064DRAFT_scaffold_4.5_C, whole |
| | genome shotgun sequence |
| 1449 | Microcystis aeruginosa NIES-843 DNA, complete genome |
| 1450 | Sulfobacillus thermosulfidooxidans strain CBAR-13 Scaffold_1_CBAR13, whole |
| | genome shotgun sequence |
| 1451 | fermentation metagenome genome assembly, contig: |
| | NODE_537_length_51166_cov_18.935787, whole genome shotgun sequence |
| 1452 | Sulfobacillus thermosulfidooxidans DSM 9293 genome assembly, contig: Contig1, |
| | whole genome shotgun sequence |
| 1453 | Desulfitobacterium hafniense TCP-A DeshafDRAFT_Scaffold2.2_C7, whole |
| | genome shotgun sequence |
| 1454 | Sulfobacillus thermosulfidooxidans DSM 9293 genome assembly, contig: Contig1, |
| | whole genome shotgun sequence |
| 1455 | Sulfobacillus thermosulfidooxidans strain CBAR-13 Scaffold_1_CBAR13, whole |
| | genome shotgun sequence |
| 1456 | Sulfobacillus thermosulfidooxidans strain CBAR-13 Scaffold_1_CBAR13, whole |
| | genome shotgun sequence |
| 1457 | Thermicanus aegyptius DSM 12793 genomic scaffold TheaeDRAFT_scaffold1.1, |
| | whole genome shotgun sequence |
| 1458 | Desulfitobacterium hafniense TCP-A genomic scaffold DeshafDRAFT_Scaffold2.2, |
| | whole genome shotgun sequence |
| 1459 | Sulfobacillus thermosulfidooxidans strain CBAR-13 Scaffold_1_CBAR13, whole |
| | genome shotgun sequence |
| 1460 | Desulfitobacterium hafniense TCP-A genomic scaffold DeshafDRAFT_Scaffold2.2, |
| | whole genome shotgun sequence |
| 1461 | Microcystis aeruginosa Ma_MB_F_20061100_S19 S19_183, whole genome shotgun |
| | sequence |
| 1462 | Microcystis aeruginosa PCC 9432 genomic scaffold, AAI_A_2195_scaffold26, |
| | whole genome shotgun sequence |
| 1463 | Planktothrix mougeotii NIVA-CYA 405 genomic scaffold scaffold00001, whole |
| | genome shotgun sequence |
| 1464 | Sulfobacillus thermosulfidooxidans str. Cutipay ALWJ01000037.1, whole genome |
| | shotgun sequence |
| 1465 | Microcystis aeruginosa Ma_QC_C_20070703_M131 M131_147, whole genome |
| | shotgun sequence |
| 1466 | Microcystis aeruginosa CACIAM 03 scaffold134, whole genome shotgun sequence |
| 1467 | Microcystis aeruginosa Ma_MB_F_20061100_S19D S19D_93, whole genome |
| | shotgun sequence |
| 1468 | Microcystis aeruginosa 9432 WGS project CAIH01000000 data, contig |
| | AAI_A_2195_114, whole genome shotgun sequence |
| 1469 | Microcystis aeruginosa SPC777 contig000190, whole genome shotgun sequence |
| 1470 | Microcystis sp. M_QC_C_20170808_M2Col M2Col_152, whole genome shotgun |
| | sequence |
| 1471 | Desulfitobacterium hafniense TCP-A DeshafDRAFT_Scaffold2.2_C7, whole |
| | genome shotgun sequence |
| 1472 | Microcystis aeruginosa NIES-2519 DNA, contig_51, whole genome shotgun |
| | sequence |
| 1473 | Ammonifex sp. isolate SURF_55 Ga0104751_1001767, whole genome shotgun |
| | sequence |
| 1474 | Microcystis aeruginosa L311-01 Ga0187976_10112, whole genome shotgun |
| | sequence |
| 1475 | Microcystis aeruginosa DA14 NODE_19_length_493533_cov_370.53, whole |
| | genome shotgun sequence |
| 1476 | Microcystis aeruginosa PCC 9809 genomic scaffold, AAI_H_2202_scaffold57, |
| | whole genome shotgun sequence |
| 1477 | TPA: hot springs metagenome genome assembly, contig: |
| | NODE_4281_length_5920_cov_6.676352, whole genome shotgun sequence |
| 1478 | Planktothrix agardhii NIVA-CYA 56/3 contig00145, whole genome shotgun |
| | sequence |
| 1479 | TPA_asm: Thermacetogenium sp. UBA4966 UBA4966_contig_280, whole genome |
| | shotgun sequence |
| 1480 | Deltaproteobacteria bacterium GWC2_56_8 gwc2_scaffold_8530, whole genome |
| | shotgun sequence |
| 1481 | Desulfitobacterium chlororespirans DSM 11544 genome assembly, contig: |
| | EJ42DRAFT_scaffold00015.15, whole genome shotgun sequence |
| 1482 | Microcystis aeruginosa KLA2 scaffold66_contigs = 7, whole genome shotgun |
| | sequence |
| 1483 | Desulfitobacterium chlororespirans DSM 11544 genome assembly, contig: |
| | EJ42DRAFT_scaffold00003.3, whole genome shotgun sequence |
| 1484 | fermentation metagenome genome assembly, contig: |
| | NODE_3141_length_10372_cov_57.258796, whole genome shotgun sequence |
| 1485 | Microcystis flos-aquae Ma_QC_C_20070823_S18 S18_108, whole genome shotgun |
| | sequence |
| 1486 | Microcystis aeruginosa KLA2 scaffold82_contigs = 9, whole genome shotgun |
| | sequence |
| 1487 | Microcystis aeruginosa NIES-2519 DNA, contig_51, whole genome shotgun |
| | sequence |
| 1488 | Planktothrix prolifica NIVA-CYA 540 contig00056, whole genome shotgun |
| | sequence |
| 1489 | Microcystis aeruginosa KLA2 scaffold82_contigs = 9, whole genome shotgun |
| | sequence |
| 1490 | Desulfurispora thermophila DSM 16022 B064DRAFT_scaffold_4.5_C, whole |
| | genome shotgun sequence |
| 1491 | Microcystis aeruginosa PCC 9717 genomic scaffold, AAI_B_2196_scaffold19, |
| | whole genome shotgun sequence |
| 1492 | Microcystis aeruginosa KLA2 scaffold355_contigs = 1, whole genome shotgun |
| | sequence |
| 1493 | Microcystis aeruginosa L211-11 Ga0187982_10598, whole genome shotgun |
| | sequence |
| 1494 | Desulfitobacterium chlororespirans DSM 11544 genome assembly, contig: |
| | EJ42DRAFT_scaffold00003.3, whole genome shotgun sequence |
| 1495 | Planktothrix agardhii NIVA-CYA 34 contig00030, whole genome shotgun sequence |
| 1496 | fermentation metagenome genome assembly, contig: |
| | NODE_280_length_68310_cov_4.125529, whole genome shotgun sequence |
| 1497 | Thermicanus aegyptius DSM 12793 TheaeDRAFT_scaffold1.1_C7, whole genome |
| | shotgun sequence |
| 1498 | Microcystis flos-aquae Mf_QC_C_20070823_S10D S10D_188, whole genome |
| | shotgun sequence |
| 1499 | fermentation metagenome genome assembly, contig: |
| | NODE_485_length_43130_cov_47.382426, whole genome shotgun sequence |
| 1500 | Microcystis aeruginosa NIES-88 scaffold11, whole genome shotgun sequence |
| 1501 | Microcystis flos-aquae Mf_QC_C_20070823_S20 S20_174, whole genome shotgun |
| | sequence |
| 1502 | Sulfobacillus thermosulfidooxidans str. Cutipay ALWJ01000037.1, whole genome |
| | shotgun sequence |
| 1503 | Microcystis flos-aquae Mf_QC_C_20070823_S20T S20T_205, whole genome |
| | shotgun sequence |
| 1504 | Microcystis aeruginosa LE3 Ga0066243_1114, whole genome shotgun sequence |
| 1505 | Microcystis aeruginosa KLA2 scaffold355_contigs = 1, whole genome shotgun |
| | sequence |
| 1506 | Microcystis sp. M_QC_C_20170808_M9Col M9Col_321, whole genome shotgun |
| | sequence |
| 1507 | TPA_asm: Syntrophomonas sp. isolate UBA11028 contig_5648, whole genome |
| | shotgun sequence |
| 1508 | Microcystis aeruginosa PCC 7941 genomic scaffold, AAI_D_2198_scaffold23, |
| | whole genome shotgun sequence |
| 1509 | Planktothrix mougeotii NIVA-CYA 405 contig00097, whole genome shotgun |
| | sequence |
| 1510 | Planktothrix agardhii NIVA-CYA 56/3 contig00145, whole genome shotgun |
| | sequence |
| 1511 | Microcystis aeruginosa Ma_OC_LR_19540900_S633 S633_109, whole genome |
| | shotgun sequence |
| 1512 | Microcystis aeruginosa SPC777 contig000190, whole genome shotgun sequence |
| 1513 | Microcystis aeruginosa NIES-2521 DNA, contig_150, whole genome shotgun |
| | sequence |
| 1514 | Microcystis flos-aquae Mf_QC_C_20070823_S10 S10_103, whole genome shotgun |
| | sequence |
| 1515 | Microcystis aeruginosa NIES-88 scaffold11, whole genome shotgun sequence |
| 1516 | Microcystis aeruginosa L311-01 Ga0187976_10112, whole genome shotgun |
| | sequence |
| 1517 | Microcystis aeruginosa KLA2 scaffold66_contigs = 7, whole genome shotgun |
| | sequence |
| 1518 | Planktothrix agardhii NIVA-CYA 34 contig00030, whole genome shotgun sequence |
| 1519 | Microcystis aeruginosa L211-101 Ga0188005_10580, whole genome shotgun |
| | sequence |
| 1520 | fermentation metagenome genome assembly, contig: |
| | NODE_5085_length_8223_cov_2.608839, whole genome shotgun sequence |
| 1521 | Microcystis aeruginosa 9809 WGS project CAIO01000000 data, contig |
| | AAI_H_2202_526, whole genome shotgun sequence |
| 1522 | Microcystis flos-aquae Mf_QC_C_20070823_S20D S20D_61, whole genome |
| | shotgun sequence |
| 1523 | Microcystis aeruginosa FCY-26 FCY-26_01051, whole genome shotgun sequence |

### Protein Modifications

The IsrBpolypeptide nucleases may comprise one or more modifications. As used herein, the term “modified” with regard to a IsrB polypeptide nuclease generally refers to a IsrB polypeptide nuclease having one or more modifications or mutations (including point mutations, truncations, insertions, deletions, chimeras, fusion proteins, etc.) compared to the wild type counterpart from which it is derived. By derived is meant that the derived enzyme is largely based, in the sense of having a high degree of sequence homology with, a wildtype enzyme, but that it has been mutated (modified) in some way as known in the art or as described herein.

The modified proteins, e.g., modified IsrB polypeptide nuclease may be catalytically inactive (also referred as dead). As used herein, a catalytically inactive or dead nuclease may have reduced or no nuclease activity compared to a wildtype counterpart nuclease. In some cases, a catalytically inactive or dead nuclease may not have nickase activity. Such a catalytically inactive or dead nuclease may not make a single-strand break on a target polynucleotide, but may still bind or otherwise form complex with the target polynucleotide.

In an embodiment, the IsrB comprises one or more mutations in the RuvC-II of the polypeptide. In an embodiment, the IsrB polypeptide comprises a mutation of the catalytic RuvC-II residue corresponding to E157 to alanine (E157A) in A. warmingii. In an aspect, the mutation of a catalytic RuvC-II residue abolishes the nickase activity on the non-target DNA strand. In an aspect, the IsrB comprises a mutation corresponding to E157A of A. warmingii, or corresponding to the positions according to consensus sequence numbering relative to A. warmingii. In an embodiment, mutation at the RuvC domain abolishes all dsDNA nucleolytic activity, providing a dead IsrB polypeptide (dIsrB). In one embodiment, the nucleolytic activity that is abolished comprises nickase activity.

In one embodiment, the modifications of the IsrB polypeptide may or may not cause an altered functionality. By means of example, modifications which do not result in an altered functionality include for instance codon optimization for expression into a particular host, or providing the nuclease with a particular marker (e.g., for visualization). Modifications which may result in altered functionality may also include mutations, including point mutations, insertions, deletions, truncations (including split nucleases), etc., as well as chimeric nucleases (e.g., comprising domains from different orthologues or homologues) or fusion proteins. A chimeric enzyme can comprise a first fragment and a second fragment, and the fragments can be of IsrB polypeptide nuclease orthologs of organisms of a genus or of a species, e.g., the fragments may be from IsrB polypeptide nuclease orthologs of different species. Fusion proteins may without limitation include, for instance, fusions with heterologous domains or functional domains (e.g., localization signals, catalytic domains, etc.). In an embodiment, various different modifications may be combined (e.g., a mutated nuclease which is catalytically inactive and which further is fused to a functional domain, such as for instance to induce DNA methylation or another nucleic acid modification, such as including without limitation, a break (e.g. by a different nuclease (domain)), a mutation, a deletion, an insertion, a replacement, a ligation, a digestion, a break or a recombination). As used herein, “altered functionality” includes without limitation an altered specificity (e.g., altered target recognition, increased (e.g., “enhanced” IsrB polypeptide nuclease) or decreased specificity, or altered TAM recognition), altered activity (e.g., increased or decreased catalytic activity, including catalytically inactive nucleases or nickases), and/or altered stability (e.g., fusions with destabilization domains). Examples of all these modifications are known in the art. It will be understood that a “modified” nuclease as referred to herein, and in particular a “modified” IsrB polypeptide comprises a “modified” nickase activity or system or complex preferably still has the capacity to interact with or bind to the polynucleic acid (e.g., in complex with the ωRNA molecule). Such modified IsrB polypeptide nickasecan be combined with the deaminase protein or active domain thereof as described herein.

In one embodiment, unmodified IsrB polypeptide nucleases may have cleavage activity. In one embodiment, the IsrB polypeptide nucleases may direct cleavage of one DNA strand at the location of or near a target sequence, such as within the target sequence and/or within the complement of the target sequence or at sequences associated with the target sequence. In one embodiment, the IsrB polypeptide nucleases may direct cleavage of one strand within about 1, 2, 3, 4, 5, 6, 7, 8, 9, 10, 15, 20, 25, 50, 100, 200, 500, or more base pairs or nucleotides from the first or last nucleotide of a target sequence. In one embodiment, the cleavage may be staggered, i.e., generating sticky ends. In one embodiment, the cleavage is a staggered cut with a 5′ overhang. In one embodiment, the cleavage is a staggered cut with a 5′ overhang of 1 to 15 nucleotides, preferably of 4 or 9 nucleotides.

As a further example, two or more catalytic domains of a IsrB polypeptide nuclease (e.g., RuvC-I, RuvC-II, and RuvC-III subdomains) may be mutated to produce a mutated IsrB polypeptide nuclease substantially lacking all DNA cleavage activity. In one embodiment, the IsrB DNA cleavage activity that is lacking is nickase activity. As described herein, corresponding catalytic domains of a IsrB polypeptide nuclease may also be mutated to produce a mutated IsrB polypeptide nuclease lacking all DNA cleavage activity or having substantially reduced DNA cleavage activity. In one embodiment, the DNA cleavage activity that is lacking is DNA nickase activity. In one embodiment, an IsrB polypeptide nuclease may be considered to substantially lacking all polynucleotide cleavage activity when the polynucleotide cleavage activity of the mutated enzyme is no more than 25%, no more than 10%, no more than 5%, no more than 1%, no more than 0.1%, no more than 0.01% of the nucleic acid cleavage activity of the non-mutated form of the enzyme; an example can be when the nucleic acid cleavage activity of the mutated form is nil or negligible as compared with the non-mutated form. In one embodiment, the IsrB polypeptide nuclease activity that is lacking is DNA nickase activity.

In an embodiment, the IsrB polypeptide nuclease may comprise one or more modifications resulting in enhanced activity and/or specificity, such as including mutating residues that stabilize the targeted or non-targeted strand. In an embodiment, the altered or modified activity of the engineered IsrB polypeptide nuclease comprises increased targeting efficiency or decreased off-target binding. In an embodiment, the altered activity of the engineered IsrB polypeptide nuclease comprises modified cleavage activity. In an embodiment, the altered activity comprises increased cleavage activity as to the target polynucleotide loci. In an embodiment, the altered activity comprises decreased cleavage activity as to the target polynucleotide loci. In an embodiment, the altered activity comprises decreased cleavage activity as to off-target polynucleotide loci. In an embodiment, the altered or modified activity of the modified nuclease comprises altered helicase kinetics. In an embodiment, the modified nuclease comprises a modification that alters association of the protein with the nucleic acid molecule comprising RNA, or a strand of the target polynucleotide loci, or a strand of off-target polynucleotide loci. In an aspect of the invention, the engineered IsrB polypeptide nuclease comprises a modification that alters formation of the IsrB polypeptide nuclease and related complex. In an embodiment, the altered activity comprises increased cleavage activity as to off-target polynucleotide loci. Accordingly, in an embodiment, there is increased specificity for target polynucleotide loci as compared to off-target polynucleotide loci. In other embodiments, there is reduced specificity for target polynucleotide loci as compared to off-target polynucleotide loci. In an embodiment, the mutations result in decreased off-target effects (e.g., cleavage or binding properties, activity, or kinetics), such as in case for IsrB polypeptide nuclease for instance resulting in a lower tolerance for mismatches between target and ωRNA. Other mutations may lead to increased off-target effects (e.g., cleavage or binding properties, activity, or kinetics). Other mutations may lead to increased or decreased on-target effects (e.g., cleavage or binding properties, activity, or kinetics). In an embodiment, the mutations result in altered (e.g., increased or decreased) helicase activity, association or formation of the functional nuclease complex. In an embodiment, the mutations result in an altered TAM recognition, i.e., a different TAM may be (in addition or in the alternative) be recognized, compared to the unmodified IsrB polypeptide nuclease. Examples mutations include positively charged residues and/or (evolutionary) conserved residues, such as conserved positively charged residues, in order to enhance specificity. In an embodiment, such residues may be mutated to uncharged residues, such as alanine.

### Nuclear Localization Sequences

In one embodiment, the nucleic acid-guided nuclease is fused to one or more nuclear localization sequences (NLSs), such as about or more than about 1, 2, 3, 4, 5, 6, 7, 8, 9, 10, or more NLSs. In one embodiment, the Nucleic acid-guided nuclease comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. zero or at least one or more NLS at the amino-terminus and zero or at one or more NLS at the carboxy terminus).

In one embodiment, the IsrBpolypeptide nuclease is fused to one or more nuclear localization sequences (NLSs), such as about or more than about 1, 2, 3, 4, 5, 6, 7, 8, 9, 10, or more NLSs. In one embodiment, the IscB polypeptide nuclease comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. zero or at least one or more NLS at the amino-terminus and zero or at one or more NLS at the carboxy terminus).

When more than one NLS is present, each may be selected independently of the others, such that a single NLS may be present in more than one copy and/or in combination with one or more other NLSs present in one or more copies. In a preferred embodiment of the invention, the Nucleic acid-guided nuclease comprises at most 6 NLSs. In a preferred embodiment of the invention, the IscB polypeptide nuclease comprises at most 6 NLSs.

In one embodiment, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Non-limiting examples of NLSs include an NLS sequence derived from: the NLS of the SV40 virus large T-antigen, having the amino acid sequence PKKKRKV (SEQ ID NO: 1527); the NLS from nucleoplasmin (e.g. the nucleoplasmin bipartite NLS with the sequence KRPAATKKAGQAKKKK (SEQ ID NO: 1528); the c-myc NLS having the amino acid sequence PAAKRVKLD (SEQ ID NO: 1529) or RQRRNELKRSP (SEQ ID NO: 1530); the hRNPA1 M9 NLS having the sequence NQSSNFGPMKGGNFGGRSSGPYGGGGQYFAKPRNQGGY (SEQ ID NO: 1531); the sequence RMRIZFKNKGKDTAELRRRRVEVSVELRKAKKDEQILKRRNV (SEQ ID NO: 1532) of the IBB domain from importin-alpha; the sequences VSRKRPRP (SEQ ID NO: 1533) and PPKKARED (SEQ ID NO: 1534) of the myoma T protein; the sequence PQPKKKPL (SEQ ID NO: 1535) of human p53; the sequence SALIKKKKKMAP (SEQ ID NO: 1536) of mouse c-abl IV; the sequences DRLRR (SEQ ID NO: 1537) and PKQKKRK (SEQ ID NO: 1538) of the influenza virus NS1; the sequence RKLKKKIKKL (SEQ ID NO: 1539) of the Hepatitis virus delta antigen; the sequence REKKKFLKRR (SEQ ID NO: 1540) of the mouse Mx1 protein; the sequence KRKGDEVDGVDEVAKKKSKK (SEQ ID NO: 1541) of the human poly (ADP-ribose) polymerase; and the sequence RKCLQAGMNLEARKTKK (SEQ ID NO: 1542) of the steroid hormone receptors (human) glucocorticoid.

In general, the one or more NLSs are of sufficient strength to drive accumulation of the nucleic acid-guided nuclease in a detectable amount in the nucleus of a eukaryotic cell. In general, strength of nuclear localization activity may derive from the number of NLSs in the nucleic acid-guided nuclease, the particular NLS(s) used, or a combination of these factors. Detection of accumulation in the nucleus may be performed by any suitable technique. For example, a detectable marker may be fused to the nucleic acid-guided nuclease, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay.

Accumulation in the nucleus may also be determined indirectly, such as by an assay for the effect of complex formation (e.g., assay for DNA cleavage or mutation at the target sequence, or assay for altered gene expression activity affected by complex formation and/or nucleic acid-guided nuclease activity), as compared to a control no exposed to the nucleic acid-guided nuclease or complex, or exposed to a nucleic acid-guided nuclease lacking the one or more NLSs. In an embodiment of the herein described nucleic acid-guided nuclease protein complexes and systems the codon optimized nucleic acid-guided nuclease proteins comprise an NLS attached to the C-terminal of the protein. In an embodiment, other localization tags may be fused to the nucleic acid-guided nuclease, such as without limitation for localizing the nucleic acid-guided nuclease to particular sites in a cell, such as organelles, such as mitochondria, plastids, chloroplast, vesicles, golgi, (nuclear or cellular) membranes, ribosomes, nucleolus, ER, cytoskeleton, vacuoles, centrosome, nucleosome, granules, centrioles, etc.

In general, the one or more NLSs are of sufficient strength to drive accumulation of the IscB polypeptide nuclease in a detectable amount in the nucleus of a eukaryotic cell. In general, strength of nuclear localization activity may derive from the number of NLSs in the IscB polypeptide nuclease, the particular NLS(s) used, or a combination of these factors. Detection of accumulation in the nucleus may be performed by any suitable technique. For example, a detectable marker may be fused to the IscB polypeptide nuclease, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of complex formation (e.g., assay for DNA cleavage or mutation at the target sequence, or assay for altered gene expression activity affected by complex formation and/or IsrB polypeptide nuclease activity), as compared to a control not exposed to the IsrB polypeptide nuclease or complex, or exposed to a IsrB polypeptide nuclease lacking the one or more NLSs. In an embodiment of the herein described IsrB polypeptide nuclease protein complexes and systems the codon optimized IsrB polypeptide nuclease proteins comprise an NLS attached to the C-terminal of the protein. In an embodiment, other localization tags may be fused to the IsrB polypeptide nuclease, such as without limitation for localizing the IsrB polypeptide nuclease to particular sites in a cell, such as organelles, such as mitochondria, plastids, chloroplast, vesicles, golgi, (nuclear or cellular) membranes, ribosomes, nucleolus, ER, cytoskeleton, vacuoles, centrosome, nucleosome, granules, centrioles, etc.

In an embodiment of the invention, at least one nuclear localization signal (NLS) is attached to the nucleic acid sequences encoding the IsrB polypeptide nuclease. In preferred embodiments at least one or more C-terminal or N-terminal NLSs are attached (and hence nucleic acid molecule(s) coding for the IsrB polypeptide nuclease can include coding for NLS(s) so that the expressed product has the NLS(s) attached or connected). In a preferred embodiment a C-terminal NLS is attached for optimal expression and nuclear targeting in eukaryotic cells, preferably human cells. The invention also encompasses methods for delivering multiple nucleic acid components, wherein each nucleic acid component is specific for a different target locus of interest thereby modifying multiple target loci of interest. The nucleic acid component of the complex may comprise one or more protein-binding RNA aptamers. The one or more aptamers may be capable of binding a bacteriophage coat protein.

### IsrBs Modified With Heterologous Functional Domains

The IsrB polypeptide (including variants such as a catalytically inactive form) may be associated with one or more functional domains (e.g., via fusion protein or suitable linkers). In an embodiment, the IsrB polypeptide nuclease, or an ortholog or homolog thereof, may be used as a generic nucleic acid binding protein with fusion to or being operably linked to one or more functional domains. In one example, the functional domain is a deaminase. In another example, the functional domain is a transposase. In another example, the functional domain is a reverse transcriptase. In some cases, a functional domain may be associate with (e.g., fuse to) the IsrBpolypeptide nuclease. In some cases, a functional domain may be a protein different from the IsrBpolypeptide nuclease. In such cases, a functional domain and the IsrB polypeptide nuclease may form a protein complex.

It is also envisaged that the IsrB complex may be associated with two or more functional domains. For example, there may be two or more functional domains associated with the IsrB polypeptide, or there may be two or more functional domains associated with the ωRNA (via one or more adaptor proteins), or there may be one or more functional domains associated with the IsrB polypeptide and one or more functional domains associated with the ωRNA (via one or more adaptor proteins).

In one embodiment, the IsrB polypeptide nuclease is associated with one or more functional domains. The association can be by direct linkage of the effector protein to the functional domain, or by association with the ωRNA. In a non-limiting example, the ωRNA comprises an added or inserted sequence that can be associated with a functional domain of interest, including, for example, an aptamer or a nucleotide that binds to a nucleic acid binding adapter protein. The functional domain may be a functional heterologous domain.

In one embodiment, the invention also provides for the one or more heterologous functional domains to have one or more of the following activities: methylase activity, demethylase activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity and nucleic acid binding activity. At least one or more heterologous functional domains may be at or near the amino-terminus of the effector protein and/or wherein at least one or more heterologous functional domains is at or near the carboxy-terminus of the effector protein. The one or more heterologous functional domains may be fused to the effector protein. The one or more heterologous functional domains may be tethered to the effector protein. The one or more heterologous functional domains may be linked to the effector protein by a linker moiety.

In an embodiment, the IsrB polypeptide nuclease or an ortholog or homolog thereof, may be used as a generic nucleic acid binding protein with fusion to or being operably linked to a functional domain. Exemplary functional domains may include but are not limited to translational initiator, translational activator, translational repressor, nucleases, in particular ribonucleases, a spliceosome, beads, a light inducible/controllable domain or a chemically inducible/controllable domain. In an embodiment, the one or more functional domains are controllable, e.g., inducible.

In one embodiment, one or more functional domains are associated with a IsrB polypeptide nuclease via an adaptor protein, for example as used with the modified guides of Konnerman et al. (Nature 517, 583-588, 29 Jan. 2015).

In one embodiment, the one or more functional domains is attached to the adaptor protein so that upon binding of the IsrB complex to the target polynucleotide, the functional domain is in a spatial orientation allowing for the functional domain to function in its attributed function.

In one embodiment, one or more functional domains are associated with a dead ωRNA molecule. In one embodiment, a ωRNA complex with active IsrB polypeptide nuclease directs gene regulation by a functional domain at on gene locus while an ωRNA directs DNA cleavage by the active IsrB polypeptide nuclease at another locus. In one embodiment, ωRNA are selected to maximize selectivity of regulation for a gene locus of interest compared to off-target regulation. In one embodiment, ωRNA are selected to maximize target gene regulation and minimize target cleavage.

For the purposes of the following discussion, reference to a functional domain could be a functional domain associated with the IsrB polypeptide nuclease or a functional domain associated with the adaptor protein. In one embodiment, the one or more functional domains is attached to the adaptor protein so that upon binding of the IsrB polypeptide nuclease to the hRNA molecule and target, the functional domain is in a spatial orientation allowing for the functional domain to function in its attributed function.

In the practice of the invention, loops of the ωRNA may be extended, without colliding with the IsrB polypeptide nuclease by the insertion of distinct RNA loop(s) or distinct sequence(s) that may recruit adaptor proteins that can bind to the distinct RNA loop(s) or distinct sequence(s). The adaptor proteins may include but are not limited to orthogonal RNA-binding protein/aptamer combinations that exist within the diversity of bacteriophage coat proteins. A list of such coat proteins includes, but is not limited to: Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s and PRR1. These adaptor proteins or orthogonal RNA binding proteins can further recruit effector proteins or fusions which comprise one or more functional domains.

Examples of functional domains include deaminase domain, transposase domain (e.g. helitron), reverse transcriptase domain, integrase domain, recombinase domain, resolvase domain, invertase domain, protease domain, DNA methyltransferase domain, DNA hydroxylmethylase domain, RNA polymerase domains, DNA demethylase domain, histone acetylase domain, histone deacetylases domain, nuclease domain (e.g. VirD2 domain), repressor domain, activator domain, nuclear-localization signal domains, transcription-regulatory protein (or transcription complex recruiting) domain, cellular uptake activity associated domain, nucleic acid binding domain, antibody presentation domain, histone modifying enzymes, recruiter of histone modifying enzymes; inhibitor of histone modifying enzymes, histone methyltransferase, histone demethylase, histone kinase, histone phosphatase, histone ribosylase, histone deribosylase, histone ubiquitinase, histone deubiquitinase, histone biotinase and histone tail protease. In some preferred embodiments, the functional domain is a transcriptional activation domain, such as, without limitation, VP64, p65, MyoD1, HSF1, RTA, SET7/9 or a histone acetyltransferase. In one embodiment, the functional domain is a transcription repression domain, preferably KRAB. In one embodiment, the transcription repression domain is SID, or concatemers of SID (e.g. SID4X). In one embodiment, the functional domain is an epigenetic modifying domain, such that an epigenetic modifying enzyme is provided. In one embodiment, the functional domain is an activation domain, which may be the P65 activation domain.

In some examples, the IsrB polypeptide nuclease is associated with a ligase or functional fragment thereof. The ligase may ligate a single-strand break (a nick) generated by the IsrB polypeptide nuclease. In certain examples, the IsrB polypeptide nuclease is associated with a reverse transcriptase or functional fragment thereof.

In one embodiment, the one or more functional domains is a transcriptional repressor domain. In one embodiment, the transcriptional repressor domain is a KRAB domain. In one embodiment, the transcriptional repressor domain is a NuE domain, NcoR domain, SID domain or a SID4X domain.

In one embodiment, the one or more functional domains have one or more activities, e.g., one or more of transposase activity, methylase activity, demethylase activity, translation activation activity, translation repression activity, transcription activation activity, transcription repression activity, transcription release factor activity, chromatin modifying or remodeling activity, histone modification activity, nuclease activity, single-strand RNA cleavage activity, double-strand RNA cleavage activity, single-strand DNA cleavage activity, double-strand DNA cleavage activity, nucleic acid binding activity, and detectable activity.

Histone modifying domains are also preferred in one embodiment. Exemplary histone modifying domains are discussed below. Transposase domains, HR (Homologous Recombination) machinery domains, recombinase domains, and/or integrase domains are also preferred as the present functional domains. In one embodiment, DNA integration activity includes HR machinery domains, integrase domains, recombinase domains and/or transposase domains.

In one embodiment, the DNA cleavage activity is due to a nuclease. In one embodiment, the nuclease comprises a Fok1 nuclease. See, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32 (6): 569-77 (2014), relates to dimeric RNA-guided FokI Nucleases that recognize extended sequences and can edit endogenous genes with high efficiencies in human cells.

In one embodiment, the one or more functional domains is attached to the IsrB polypeptide nuclease so that upon binding to the sgRNA and target the functional domain is in a spatial orientation allowing for the functional domain to function in its attributed function.

In one embodiment, the IsrB polypeptide nuclease comprise one or more heterologous functional domains. As used herein, a heterologous functional domain is a polypeptide that is not derived from the same species as the IsrB polypeptide nuclease. For example, a heterologous functional domain of a IsrB polypeptide nuclease derived from species A is a polypeptide derived from a species different from species A, or an artificial polypeptide. The one or more heterologous functional domains may comprise one or more nuclear localization signal (NLS) domains. The one or more heterologous functional domains may comprise at least two or more NLSs. The one or more heterologous functional domains may comprise one or more transcriptional activation domains. A transcriptional activation domain may comprise VP64. The one or more heterologous functional domains may comprise one or more transcriptional repression domains. A transcriptional repression domain may comprise a KRAB domain or a SID domain. The one or more heterologous functional domain may comprise one or more nuclease domains. The one or more nuclease domains may comprise Fok1.

Functional domains may be used to regulate transcription, e.g., transcriptional repression. Transcriptional repression is often mediated by chromatin modifying enzymes such as histone methyltransferases (HMTs) and deacetylases (HDACs). Repressive histone effector domains are known and an exemplary list is provided below. In the exemplary table, preference was given to proteins and functional truncations of small size to facilitate efficient viral packaging (for instance via AAV). In general, however, the domains may include HDACs, histone methyltransferases (HMTs), and histone acetyltransferase (HAT) inhibitors, as well as HDAC and HMT recruiting proteins. The functional domain may be or include, In one embodiment, HDAC Effector Domains, HDAC Recruiter Effector Domains, Histone Methyltransferase (HMT) Effector Domains, Histone Methyltransferase (HMT) Recruiter Effector Domains, or Histone Acetyltransferase Inhibitor Effector Domains.

In one embodiment, the functional domain may be a Methyltransferase (HMT) Effector Domain. Preferred examples include NUE, vSET, EHMT2/G9A, SUV39H1, dim-5, KYP, SUVR4, SET4, SET1, SETD8, and TgSET8. NUE is exemplified in the present Examples and, although preferred, it is envisaged that others in the class will also be useful.

In one embodiment, the functional domain may be a Histone Methyltransferase (HMT) Recruiter Effector Domain. Preferred examples include Hp1a, PHF19, and NIPP1.

In one embodiment, the functional domain may be Histone Acetyltransferase Inhibitor Effector Domain. Preferred examples include SET/TAF-1β.

In some cases, the target endogenous (regulatory) control elements (such as enhancers and silencers) in addition to a promoter or promoter-proximal elements. Thus, the invention can also be used to target endogenous control elements (including enhancers and silencers) in addition to targeting of the promoter. These control elements can be located upstream and downstream of the transcriptional start site (TSS), starting from 200 bp from the TSS to 100 kb away. Targeting of known control elements can be used to activate or repress the gene of interest. In some cases, a single control element can influence the transcription of multiple target genes. Targeting of a single control element could therefore be used to control the transcription of multiple genes simultaneously.

Targeting of putative control elements on the other hand (e.g. by tiling the region of the putative control element as well as 200 bp up to 100 kB around the element) can be used as a means to verify such elements (by measuring the transcription of the gene of interest) or to detect novel control elements (e.g. by tiling 100 kb upstream and downstream of the TSS of the gene of interest). In addition, targeting of putative control elements can be useful in the context of understanding genetic causes of disease. Many mutations and common SNP variants associated with disease phenotypes are located outside coding regions. Targeting of such regions with either the activation or repression systems described herein can be followed by readout of transcription of either a) a set of putative targets (e.g. a set of genes located in closest proximity to the control element) or b) whole-transcriptome readout by e.g. RNAseq or microarray. This would allow for the identification of likely candidate genes involved in the disease phenotype. Such candidate genes could be useful as novel drug targets.

In one embodiment is for the one or more functional domains to comprise an acetyltransferase, preferably a histone acetyltransferase. These are useful in the field of epigenomics, for example in methods of interrogating the epigenome. Methods of interrogating the epigenome may include, for example, targeting epigenomic sequences. Targeting epigenomic sequences may include the hRNA being directed to an epigenomic target sequence. Epigenomic target sequence may include, in one embodiment, include a promoter, silencer or an enhancer sequence.

The functional domains may be acetyltransferases domains. Examples of acetyltransferases are known but may include, In one embodiment, histone acetyltransferases. In one embodiment, the histone acetyltransferase may comprise the catalytic core of the human acetyltransferase p300 (Gerbasch & Reddy, Nature Biotech 6th April 2015).

### Linkers

In an embodiment of the invention, at least one nuclear localization signal (NLS) is attached to the nucleic acid sequences encoding the nucleic acid-guided nuclease or the IscB polypeptide nuclease. In preferred embodiments at least one or more C-terminal or N-terminal NLSs are attached (and hence nucleic acid molecule(s) coding for the nucleic acid-guided nuclease or IsrB polypeptide nuclease can include coding for NLS(s) so that the expressed product has the NLS(s) attached or connected). In a preferred embodiment a C-terminal NLS is attached for optimal expression and nuclear targeting in eukaryotic cells, preferably human cells. The invention also encompasses methods for delivering multiple nucleic acid components, wherein each nucleic acid component is specific for a different target locus of interest thereby modifying multiple target loci of interest. The nucleic acid component of the complex may comprise one or more protein-binding RNA aptamers. The one or more aptamers may be capable of binding a bacteriophage coat protein.

In some preferred embodiments, the functional domain is linked to a nucleic acid-guided nuclease (e.g., an active or a dead nucleic acid-guided nuclease) to target and activate epigenomic sequences such as promoters or enhancers. One or more guides directed to such promoters or enhancers may also be provided to direct the binding of the nucleic acid-guided nuclease to such promoters or enhancers.

In some preferred embodiments, the functional domain is linked to a IsrB polypeptide nuclease (e.g., an active or a dead IsrB polypeptide nuclease) to target and activate epigenomic sequences such as promoters or enhancers. One or more guides directed to such promoters or enhancers may also be provided to direct the binding of the IsrB polypeptide nuclease to such promoters or enhancers.

The term “associated with” is used here in relation to the association of the functional domain to the IscB polypeptide nuclease protein, nucleic acid-guided nuclease, or the adaptor protein. It is used in respect of how one molecule ‘associates’ with respect to another, for example between an adaptor protein and a functional domain, between the IscB polypeptide nuclease protein and a functional domain, or between the nucleic acid guided nuclease protein and a functional domain. In the case of such protein-protein interactions, this association may be viewed in terms of recognition in the way an antibody recognizes an epitope. Alternatively, one protein may be associated with another protein via a fusion of the two, for instance one subunit being fused to another subunit. Fusion typically occurs by addition of the amino acid sequence of one to that of the other, for instance via splicing together of the nucleotide sequences that encode each protein or subunit. Alternatively, this may essentially be viewed as binding between two molecules or direct linkage, such as a fusion protein. In any event, the fusion protein may include a linker between the two subunits of interest (i.e. between the enzyme and the functional domain or between the adaptor protein and the functional domain). Thus, in one embodiment, the IsrB polypeptide nuclease protein, nucleic acid-guided nuclease, or adaptor protein is associated with a functional domain by binding thereto. In other embodiments, the IscB polypeptide nuclease, nucleic acid-guided nuclease, or adaptor protein is associated with a functional domain because the two are fused together, optionally via an intermediate linker.

The term “linker” as used in reference to a fusion protein refers to a molecule which joins the proteins to form a fusion protein. Generally, such molecules have no specific biological activity other than to join or to preserve some minimum distance or other spatial relationship between the proteins. However, in an embodiment, the linker may be selected to influence some property of the linker and/or the fusion protein such as the folding, net charge, or hydrophobicity of the linker.

Suitable linkers for use in the methods of the present invention are well known to those of skill in the art and include, but are not limited to, straight or branched-chain carbon linkers, heterocyclic carbon linkers, or peptide linkers. However, as used herein the linker may also be a covalent bond (carbon-carbon bond or carbon-heteroatom bond).

In one embodiment, the linker is used to separate the IsrB polypeptide nuclease and the nucleotide deaminase by a distance sufficient to ensure that each protein retains its required functional property. In one embodiment, the linker is used to separate the nucleic acid-guided nuclease and the nucleotide deaminase by a distance sufficient to ensure that each protein retains its required functional property.

For example, GlySer linkers GGS, GGGS (SEQ ID NO: 1543) or GSG can be used. GGS, GSG, GGGS (SEQ ID NO: 1543) or GGGGS (SEQ ID NO: 1544) linkers can be used in repeats of 3 (such as (GGS)₃, (SEQ ID NO: 1545) (GGGGS)₃) (SEQ ID NO: 1546) or 5, 6, 7, 9 or even 12 or more, to provide suitable lengths. In some cases, the linker may be (GGGGS)₃₋₁₅, For example, in some cases, the linker may be (GGGGS)₃₋₁₁, e.g., GGGGS (SEQ ID NO: 1544), (GGGGS)₂ (SEQ ID NO: 1547), (GGGGS); (SEQ ID NO: 1546), (GGGGS)+ (SEQ ID NO: 1548), (GGGGS)₅ (SEQ ID NO: 1549), (GGGGS)₆ (SEQ ID NO: 1550), (GGGGS)₇ (SEQ ID NO: 1551), (GGGGS): (SEQ ID NO: 1552), (GGGGS)₉ (SEQ ID NO: 1553), (GGGGS)₁₀ (SEQ ID NO: 1554), or (GGGGS)₁₁ (SEQ ID NO: 1555).

In one embodiment, linkers such as (GGGGS); (SEQ ID NO: 1546) are preferably used herein. (GGGGS)₆ (SEQ ID NO: 1550), (GGGGS)₉ (SEQ ID NO: 1553) or (GGGGS)₁₂ (SEQ ID NO: 1556) may preferably be used as alternatives. Other preferred alternatives are (GGGGS) | (SEQ ID NO: 1544), (GGGGS)₂ (SEQ ID NO: 1547), (GGGGS)+ (SEQ ID NO: 1548), (GGGGS)₅ (SEQ ID NO: 1549), (GGGGS)₇ (SEQ ID NO: 1551), (GGGGS)₈ (SEQ ID NO: 1552), (GGGGS)₁₀ (SEQ ID NO: 1554), or (GGGGS)₁₁ (SEQ ID NO: 1555). In yet a further embodiment, LEPGEKPYKCPECGKSFSQSGALTRHQRTHTR (SEQ ID NO: 1557) is used as a linker. In yet an additional embodiment, the linker is an XTEN linker. In one embodiment, the IsrB polypeptide nuclease or the nucleic acid-guided nuclease is linked to the deaminase protein or its catalytic by domain means of an LEPGEKPYKCPECGKSFSQSGALTRHQRTHTR (SEQ ID NO: 1557) linker. In further one embodiment, IsrB polypeptide nuclease is linked C-terminally to the N-terminus of a deaminase protein or its catalytic by domain means of an LEPGEKPYKCPECGKSFSQSGALTRHQRTHTR (SEQ ID NO: 1557) linker. In addition, N- and C-terminal NLSs can also function as linker (e.g., PKKKRKVEASSPKKRKVEAS (SEQ ID NO: 1558)).

TABLE 2
Examples of linkers used in the invention are
shown.

| | |
|---|---|
| GGS | GGTGGTAGT |
| GGSx3 (9) | GGTGGTAGTGGAGGGAGCGGCGGTTCA |
| (SEQ ID | (SEQ ID NO: 1560) |
| NO: 1545) | |
| GGSx7 (21) | ggtggaggaggctctggtggaggcggtagcggaggcgg |
| (SEQ ID | agggtcgGGTGGTAGTGGAGGGAGCGGCGGTTCA |
| NO: 1559) | (SEQ ID NO: 1561) |
| XTEN | TCGGGATCTGAGACGCCTGGGACCTCGGAATCGGCTAC |
| | GCCCGAAAGT (SEQ ID NO: 1562) |
| Z-EGFR_ | Gtggataacaaatttaacaaagaaatgtgggcggcgtg |
| Short | ggaagaaattcgtaacctgccgaacctgaacggctggc |
| | agatgaccgcgtttattgcgagcctggtggatgatccg |
| | agccagagcgcgaacctgctggcggaagcgaaaaaact |
| | gaacgatgcgcaggcgccgaaaaccggcggtggttctg |
| | gt (SEQ ID NO: 1563) |
| GSAT | Ggtggttctgccggtggctccggttctggctccagcgg |
| | tggcagctctggtgcgtccggcacgggtactgcgggtg |
| | gcactggcagcggttccggtactggctctggc |
| | (SEQ ID NO: 1564) |

Linkers may be used between the hRNA molecules and the functional domain (activator or repressor), or between the IscB polypeptide nuclease and the functional domain. In an embodiment, linkers may be used between the guide molecules and the functional domain (e.g. activator or repressor), or between the Cas IsrB polypeptide nuclease and the functional domain. The linkers may be used to engineer appropriate amounts of “mechanical flexibility”.

In an embodiment, the one or more functional domains are controllable, e.g., inducible.

### ωRNAs

The systems herein may further comprise one or more ωRNA molecules, which are referred to herein interchangeably as ωRNA. The ωRNA complex can comprise a guide sequence and a scaffold that interacts with the IsrB polypeptide. An ωRNA molecule may form a complex with IsrB polypeptide nuclease or IsrB polypeptide, and direct sequence-specific binding of the complex to a target sequence on a target polypeptide.

In certain example embodiments, the ωRNA molecule is a single molecule comprising a scaffold sequence and a spacer sequence. In certain example embodiments, the spacer is 5′ of the scaffold sequence. In certain example embodiments, the ωRNA molecule may further comprise a conserved nucleic acid sequence between the scaffold and spacer portions. As used herein, “target sequence” refers to a sequence to which a guide sequence is designed to have complementarity, where hybridization between a target sequence and a guide RNA promotes the formation of a DNA or RNA-targeting complex. Full complementarity is not necessarily required, provided there is sufficient complementarity to cause hybridization and promote formation of a nucleic acid-targeting complex. A target sequence may comprise RNA polynucleotides. In one embodiment, a target sequence is located in the nucleus or cytoplasm of a cell. In one embodiment, the target sequence may be within an organelle of a eukaryotic cell, for example, mitochondrion or chloroplast. A sequence or template that may be used for recombination into the targeted locus comprising the target sequences is referred to as an “editing template” or “editing sequence”. In aspects of the invention, an exogenous template may be referred to as an editing template. In an aspect the recombination is homologous recombination.

In certain example embodiments, the ωRNA scaffold comprises a spacer sequence and a conserved nucleotide sequence. The ωRNA scaffold typically comprises conserved regions, with the scaffold comprising 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 115, 125, 135, 145, 155, 165, 175, 185, 195, 205, 215, 225, 235, 245, 255, 265, 275, 285, 295, 305, 315, 325, 335, 345, or 355 or more nt. In an aspect, the ωRNA scaffold comprises one conserved nucleotide sequence. In embodiments, the conserved nucleotide sequence is on or near a 5′ end of the scaffold. In embodiments, the scaffold may comprise a short 3-4 base pair nexus, a conserved nexus hairpin and a large multi-stem loop region that may consist of two interconnected multi-stem loops. In an aspect, an IsrB associated scaffold may comprise a spacer, which can be re-programmed to direct site-specific binding to a target sequence of a target polynucleotide. The spacer may also be referred to herein as part of the ωRNA scaffold or as gRNA, and may comprise an engineered heterologous sequence.

In an embodiment, the spacer length of the ωRNA is from 10 to 150 nt. In an embodiment, the spacer length of the guide RNA is at least 15 nucleotides. In an embodiment, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 17, 138, 19, 140, 141, 142, 143, 144, 145, 146, 147, 148, 149 or 150 nt.

In an embodiment, the ωRNA spacer length is from 15 to 50 nt. In an embodiment, the spacer length of the ωRNA is at least 15 nucleotides. In an embodiment, the spacer length is from 15 to 50 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt, from 34 to 40 nt, e.g., 34, 35, 36, 37, 38, 39, 40, from 35 to 39, from 36 to 38 nt long, about 37 nt, or longer.

In one embodiment, the sequence of the ωRNA molecule is selected to reduce the degree secondary structure within the @ RNA molecule. In one embodiment, about or less than about 75%, 50%, 40%, 30%, 25%, 20%, 15%, 10%, 5%, 1%, or fewer of the nucleotides of the nucleic acid-targeting ωRNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example of a folding algorithm is the online webserver RNAfold, developed at Institute for Theoretical Chemistry at the University of Vienna, using the centroid structure prediction algorithm (see e.g., A. R. Gruber et al., 2008, Cell 106 (1): 23-24; and PA Carr and GM Church, 2009, Nature Biotechnology 27 (12): 1151-62).

As used herein, a heterologous ωRNA molecule is an ωRNA molecule that is not derived from the same species as the IsrB polypeptide, or comprises a portion of the molecule, e.g., spacer, that is not derived from the same species as the IsrB polypeptidenuclease, e.g. IsrB protein. For example, a heterologous ωRNA molecule of a IsrB polypeptide nuclease derived from species A comprises a polynucleotide derived from a species different from species A, or an artificial polynucleotide.

In a particular embodiment, the ωRNA comprises a guide sequence linked to a conserved nucleotide sequence, wherein the conserved nucleotide sequence may comprise one or more stem loops or optimized secondary structures. In an embodiment, the conserved nucleotide sequence has a minimum length of 16 nts and a single stem loop. In further embodiments the conserved nucleotide sequence has a length longer than 16 nts, preferably more than 17 nts, and has more than one stem loops or optimized secondary structures. In one embodiment, the guide sequence may be linked to all or part of the natural conserved nucleotide sequence. In one embodiment, certain aspects of the guide architecture can be modified, for example by addition, subtraction, or substitution of features, whereas certain other aspects of guide architecture are maintained. Preferred locations for engineered guide modifications, including but not limited to insertions, deletions, and substitutions include guide termini and regions of the guide that are exposed when complexed with IsrB polypeptide nuclease and/or target, for example the tetraloop and/or loop2.

In one embodiment, a loop in the guide RNA is provided. This may be a stem loop or a tetra loop. The loop is preferably GAAA, but it is not limited to this sequence or indeed to being only 4 bp in length. Indeed, preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG.

In one embodiment, the ωRNA forms a stemloop with a separate non-covalently linked sequence, which can be DNA or RNA. In an embodiment, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In one embodiment, these sequences can be functionalized to contain an appropriate functional group for ligation using the standard protocol known in the art (Hermanson, G. T., Bioconjugate Techniques, Academic Press (2013). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sulfonyl, ally, propargyl, diene, alkyne, and azide. Once this sequence is functionalized, a covalent chemical bond or linkage can be formed between this sequence and the conserved nucleotide sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In one embodiment, these stem-loop forming sequences can be chemically synthesized. In one embodiment, the chemical synthesis uses automated, solid-phase oligonucleotide synthesis machines with 2′-acetoxyethyl orthoester (2′-ACE) (Scaringe et al., J. Am. Chem. Soc. (1998) 120:11820-11821; Scaringe, Methods Enzymol. (2000) 317:3-18) or 2′-thionocarbamate (2′-TC) chemistry (Dellinger et al., J. Am. Chem. Soc. (2011) 133:11540-11546; Hendel et al., Nat. Biotechnol. (2015) 33:985-989).

The repeat: anti-repeat duplex will be apparent from the secondary structure of the ωRNA. It may be typically a first complimentary stretch after (in 5′ to 3′ direction) the poly U tract and before the tetraloop; and a second complimentary stretch after (in 5′ to 3′ direction) the tetraloop and before the polyA tract. The first complimentary stretch (the “repeat”) is complimentary to the second complimentary stretch (the “anti-repeat”). As such, they Watson-Crick base pair to form a duplex of dsRNA when folded back on one another. As such, the anti-repeat sequence is the complimentary sequence of the repeat and in terms to A-U or C-G base pairing, but also in terms of the fact that the anti-repeat is in the reverse orientation due to the tetraloop.

As used herein, the term “spacer” may also be referred to as a “guide sequence.” In one embodiment, the degree of complementarity of the guide quence to a given target sequence, when optimally aligned using a suitable alignment algorithm, is about or more than 50%, 60%, 75%, 80%, 85%, 90%, 95%, 97.5%, 99%, or more. In certain example embodiments, the ωRNA molecule comprises a guide sequence that may be designed to have at least one mismatch with the target sequence, such that a RNA duplex formed between the sequence and the target sequence. Accordingly, the degree of complementarity is less than 99%. For instance, where the guide sequence consists of 24 nucleotides, the degree of complementarity is more particularly about 96% or less. In one embodiment, the guide sequence is designed to have a stretch of two or more adjacent mismatching nucleotides, such that the degree of complementarity over the entire sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In one embodiment, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at novocraft.com), ELAND (Illumina, San Diego, CA), SOAP (available at soap.genomics.org.cn), and Maq (available at maq.sourceforge.net). The ability of a sequence (within a nucleic acid-targeting guide sequence) to direct sequence-specific binding of a nucleic acid-targeting complex to a target nucleic acid sequence may be assessed by any suitable assay. For example, the components of a ωRNA system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the sequence to be tested and a control sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting @ RNA may be selected to target any target nucleic acid sequence.

A ωRNA sequence, and hence a nucleic acid-targeting guide, may be selected to target any target nucleic acid sequence. The target sequence may be DNA. The target sequence may be any RNA sequence. In one embodiment, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In one embodiment, the ωRNA molecule forms a stemloop with a separate non-covalently linked sequence, which can be DNA or RNA. In one embodiment, the sequences forming the @ RNA are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In one embodiment, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the conserved nucleotide sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In one embodiment, these stem-loop forming sequences can be chemically synthesized. In one embodiment, the chemical synthesis uses automated, solid-phase oligonucleotide synthesis machines with 2′-acetoxyethyl orthoester (2′-ACE) (Scaringe et al., J. Am. Chem. Soc. (1998) 120:11820-11821; Scaringe, Methods Enzymol. (2000) 317:3-18) or 2′-thionocarbamate (2′-TC) chemistry (Dellinger et al., J. Am. Chem. Soc. (2011) 133:11540-11546; Hendel et al., Nat. Biotechnol. (2015) 33:985-989).

### Chemical Modifications

In an embodiment, the ωRNA molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the ωRNA sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a ωRNA nucleic acid comprises ribonucleotides and non-ribonucleotides. In one such embodiment, a ωRNA comprises one or more ribonucleotides and one or more deoxyribonucleotides. In an embodiment of the invention, the ωRNA comprises one or more non-naturally occurring nucleotide or nucleotide analog such as a nucleotide with phosphorothioate linkage, locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of ωRNA chemical modifications include, without limitation, incorporation of 2′-O-methyl (M), 2′-O-methyl 3′phosphorothioate (MS), S-constrained ethyl (cEt), or 2′-O-methyl 3′thioPACE (MSP) at one or more terminal nucleotides. Such chemically modified ωRNA can comprise increased stability and increased activity as compared to unmodified ωRNA, though on-target vs. off-target specificity is not predictable. (See, Hendel, 2015, Nat Biotechnol. 33 (9): 985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33 (9): 985-989; Li et al., Nature Biomedical Engineering, 2017, 1, 0066 DOI: 10.1038/s41551-017-0066). In one embodiment, the 5′ and/or 3′ end of a ωRNA is modified by a variety of functional moieties including fluorescent dyes, polyethylene glycol, cholesterol, proteins, or detection tags. (See Kelly et al., 2016, J. Biotech. 233:74-83). In an embodiment, a ωRNA comprises ribonucleotides in a region that binds to a target sequence and one or more deoxyribonucletides and/or nucleotide analogs in a region that binds to the IscB polypeptide nuclease. In an embodiment, deoxyribonucleotides and/or nucleotide analogs are incorporated in engineered hRNA structures. In one embodiment, 3-5 nucleotides at either the 3′ or the 5′ end of a hRNA is chemically modified. In one embodiment, only minor modifications are introduced in the seed region, such as 2′-F modifications. In one embodiment, 2′-F modification is introduced at the 3′ end of a hRNA. In an embodiment, three to five nucleotides at the 5′ and/or the 3′ end of the hRNA are chemically modified with 2′-O-methyl (M), 2′-O-methyl 3′ phosphorothioate (MS), S-constrained ethyl (cEt), or 2′-O-methyl 3′ thioPACE (MSP). Such modification can enhance genome editing efficiency (see Hendel et al., Nat. Biotechnol. (2015) 33 (9): 985-989). In an embodiment, all of the phosphodiester bonds of a hRNA are substituted with phosphorothioates (PS) for enhancing levels of gene disruption. In an embodiment, more than five nucleotides at the 5′ and/or the 3′ end of the hRNA are chemically modified with 2′-O-Me, 2′-F or S-constrained ethyl (cEt). Such chemically modified hRNA can mediate enhanced levels of gene disruption (see Ragdarm et al., 0215, PNAS, E7110-E7111). In an embodiment of the invention, a hRNA is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the hRNA by a linker, such as an alkyl chain. In an embodiment, the chemical moiety of the modified hRNA can be used to attach the hRNA to another molecule, such as DNA, RNA, protein, or nanoparticles. Such chemically modified hRNA can be used to identify or enrich cells generically edited by a IscB polypeptide nuclease and related systems (see Lee et al., eLife, 2017, 6: e25312, DOI: 10.7554).

In a particular embodiment, the conserved nucleotide sequence may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In embodiments, the IsrB polypeptide utilizes the hRNA scaffold comprising a polynucleotide sequence that facilitates the interaction with the IsrB protein, allowing for sequence specific binding and/or targeting of the guide sequence with the target polynucleotide. Chemical synthesis of the hRNA scaffold is contemplated, using covalent linkage using various bioconjugation reactions, loops, bridges, and non-nucleotide links via modifications of sugar, internucleotide phosphodiester bonds, purine and pyrimidine residues. Sletten et al., Angew. Chem. Int. Ed. (2009) 48:6974-6998; Manoharan, M. Curr. Opin. Chem. Biol. (2004) 8:570-9; Behlke et al., Oligonucleotides (2008) 18:305-19; Watts, et al., Drug. Discov. Today (2008) 13:842-55; Shukla, et al., ChemMedChem (2010) 5:328-49; chemical synthesis using automated, solid-phase oligonucleotide synthesis machines with 2′-acetoxyethyl orthoester (2′-ACE) (Scaringe et al., J. Am. Chem. Soc. (1998) 120:11820-11821; Scaringe, Methods Enzymol. (2000) 317:3-18) or 2′-thionocarbamate (2′-TC) chemistry (Dellinger et al., J. Am. Chem. Soc. (2011) 133:11540-11546; Hendel et al., Nat. Biotechnol. (2015) 33:985-989).

In certain example embodiments, the scaffold and spacer may be designed as two separate molecules that can hybridize or covalently joined into a single molecule. Covalent linkage can be via a linker (e.g., a non-nucleotide loop) that comprises a moiety such as spacers, attachments, bioconjugates, chromophores, reporter groups, dye labeled RNAs, and non-naturally occurring nucleotide analogues. More specifically, suitable spacers for purposes of this invention include, but are not limited to, polyethers (e.g., polyethylene glycols, polyalcohols, polypropylene glycol or mixtures of efhylene and propylene glycols), polyamines group (e.g., spennine, spermidine and polymeric derivatives thereof), polyesters (e.g., poly(ethyl acrylate)), polyphosphodiesters, alkylenes, and combinations thereof. Suitable attachments include any moiety that can be added to the linker to add additional properties to the linker, such as but not limited to, fluorescent labels. Suitable bioconjugates include, but are not limited to, peptides, glycosides, lipids, cholesterol, phospholipids, diacyl glycerols and dialkyl glycerols, fatty acids, hydrocarbons, enzyme substrates, steroids, biotin, digoxigenin, carbohydrates, polysaccharides. Suitable chromophores, reporter groups, and dye-labeled RNAs include, but are not limited to, fluorescent dyes such as fluorescein and rhodamine, chemiluminescent, electrochemiluminescent, and bioluminescent marker compounds. The design of example linkers conjugating two RNA components are also described in WO 2004/015075.

The linker (e.g., a non-nucleotide loop) can be of any length. In one embodiment, the linker has a length equivalent to about 0-16 nucleotides. In one embodiment, the linker has a length equivalent to about 0-8 nucleotides. In one embodiment, the linker has a length equivalent to about 0-4 nucleotides. In one embodiment, the linker has a length equivalent to about 2 nucleotides. Example linker design is also described in International Patent Publication No. WO 2011/008730.

### Escorted ωRNA Molecules

In one embodiment, the compositions or complexes have a ωRNA molecule with a functional structure designed to improve ωRNA molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green fluorescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in one embodiment, the ωRNA molecule is modified, e.g., by one or more aptamer(s) designed to improve ωRNA molecule delivery, including delivery across the cellular membrane, to intracellular compartments, or into the nucleus. Such a structure can include, either in addition to the one or more aptamer(s) or without such one or more aptamer(s), moiety(ies) so as to render the ωRNA molecule deliverable, inducible or responsive to a selected effector. The invention accordingly comprehends a ωRNA molecule that responds to normal or pathological physiological conditions, including without limitation pH, hypoxia, O2 concentration, temperature, protein concentration, enzymatic concentration, lipid structure, light exposure, mechanical disruption (e.g., ultrasound waves), magnetic fields, electric fields, or electromagnetic radiation.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

Energy sources such as electromagnetic radiation, sound energy or thermal energy may induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm2. In a preferred embodiment, a stimulation paradigm of as low as 0.25 sec every 15 sec should result in maximal activation.

The chemical or energy sensitive hRNA may undergo a conformational change upon induction by the binding of a chemical source or by the energy allowing it act as a hRNA and have the IscB polypeptide nuclease system or complex function. The invention can involve applying the chemical source or energy so as to have the hRNA function and the IscB polypeptide nuclease system or complex function; and optionally further determining that the expression of the genomic locus is altered.

There are several different designs of this chemical inducible system: 1. ABI-PYL based system inducible by Abscisic Acid (ABA) (see, e.g., stke.sciencemag.org/cgi/content/abstract/sigtrans; 4/164/rs2), 2. FKBP-FRB based system inducible by rapamycin (or related chemicals based on rapamycin) (see, e.g., www.nature.com/nmeth/journal/v2/n6/full/nmeth763.html), 3. GID1-GAI based system inducible by Gibberellin (GA) (see, e.g., www.nature.com/nchembio/journal/v8/n5/full/nchembio.922.html).

A chemical inducible system can be an estrogen receptor (ER) based system inducible by 4-hydroxytamoxifen (40HT) (see, e.g., www.pnas.org/content/1Apr. 3, 1027.abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogen receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel-based system inducible by energy, heat or radio-wave (see, e.g., www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the hRNA and the other components of the IsrB polypeptide nuclease/hRNA molecule complex or system, and the binding will induce the change of sub-cellular localization of the polypeptide, leading to the entire polypeptide entering the nucleus of cells. Once inside the nucleus, the hRNA protein and the other components of the IsrB polypeptide nuclease/hRNA molecule complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 k Volts/cm under in vivo conditions. Instead of or in addition to the pulses, the electric field may be delivered in a continuous manner. The electric pulse may be applied for between 1 us and 500 milliseconds, preferably between 1 us and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc., as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100.mu·s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably, the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably, the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue, or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm². Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz′ (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic, and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably, the exposure to an ultrasound energy source is at a power density of from about 0.05 to about 100 Wcm-2. Even more preferably, the exposure to an ultrasound energy source is at a power density of from about 1 to about 15 Wcm-2.

Preferably, the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm-2 to about 10 Wcm-2 with a frequency ranging from about 0.015 to about 10 MHz (see WO 98/52609). However, alternatives are also possible, for example, exposure to an ultrasound energy source at an acoustic power density of above 100 Wcm-2, but for reduced periods of time, for example, 1000 Wcm-2 for periods in the millisecond range or less.

Preferably, the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In one embodiment, the hRNA molecule is modified by a secondary structure to increase the specificity of the IsrB polypeptide nuclease and related system and the secondary structure can protect against exonuclease activity and allow for 5′ additions to the hRNA sequence also referred to herein as a protected hRNA molecule.

In one aspect, the invention provides for hybridizing a “protector RNA” to a sequence of the hRNA molecule, wherein the “protector RNA” is an RNA strand complementary to the 3′ end of the hRNA molecule to thereby generate a partially double-stranded hRNA. In an embodiment of the invention, protecting mismatched bases (i.e., the bases of the hRNA molecule which do not form part of the hRNA sequence) with a perfectly complementary protector sequence decreases the likelihood of target DNA binding to the mismatched basepairs at the 3′ end. In one embodiment of the invention, additional sequences comprising an extended length may also be present within the hRNA molecule such that the hRNA comprises a protector sequence within the hRNA molecule. This “protector sequence” ensures that the hRNA molecule comprises a “protected sequence” in addition to an “exposed sequence” (comprising the part of the hRNA sequence hybridizing to the target sequence). In one embodiment, the hRNA molecule is modified by the presence of the protector hRNA to comprise a secondary structure such as a hairpin. Advantageously there are three or four to thirty or more, e.g., about 10 or more, contiguous base pairs having complementarity to the protected sequence, the hRNA sequence or both. It is advantageous that the protected portion does not impede thermodynamics of the IscB polypeptide nuclease and related system interacting with its target. By providing such an extension including a partially double stranded hRNA molecule, the hRNA molecule is considered protected and results in improved specific binding of the IscB polypeptide nuclease/hRNA molecule complex, while maintaining specific activity.

In one embodiment, use is made of a truncated hRNA (tru-hRNA), i.e., a hRNA molecule which comprises a hRNA sequence which is truncated in length with respect to the canonical hRNA sequence length. As described by Nowak et al. (Nucleic Acids Res (2016) 44 (20): 9555-9564), such guides may allow catalytically active IscB polypeptide nuclease to bind its target without cleaving the target DNA. In one embodiment, a truncated hRNA is used which allows the binding of the target but retains only nickase activity of the IscB polypeptide nuclease.

In one embodiment, conjugation of triantennary N-acetyl galactosamine (GalNAc) to oligonucleotide components may be used to improve delivery, for example delivery to select cell types, for example hepatocytes (see International Patent Publication No. WO 2014/118272 incorporated herein by reference; Nair, J K et al., 2014, Journal of the American Chemical Society 136 (49), 16958-16961). This is considered to be a sugar-based particle and further details on other particle delivery systems and/or formulations are provided herein. GalNAc can therefore be considered to be a particle in the sense of the other particles described herein, such that general uses and other considerations, for instance delivery of said particles, apply to GalNAc particles as well. A solution-phase conjugation strategy may for example be used to attach triantennary GalNAc clusters (mol. wt. ˜2000) activated as PFP (pentafluorophenyl) esters onto 5′-hexylamino modified oligonucleotides (5′-HA ASOs, mol. wt. ˜8000 Da; Østergaard et al., Bioconjugate Chem., 2015, 26 (8), pp 1451-1455). Similarly, poly (acrylate) polymers have been described for in vivo nucleic acid delivery (see WO2013158141 incorporated herein by reference). In further alternative embodiments, pre-mixing IscB polypeptide nuclease nanoparticles (or protein complexes) with naturally occurring serum proteins may be used in order to improve delivery (Akinc A et al, 2010, Molecular Therapy vol. 18 no. 7, 1357-1364).

Screening techniques are available to identify delivery enhancers, for example by screening chemical libraries (Gilleron J. et al., 2015, Nucl. Acids Res. 43 (16): 7984-8001). Approaches have also been described for assessing the efficiency of delivery vehicles, such as lipid nanoparticles, which may be employed to identify effective delivery vehicles for components (see Sahay G. et al., 2013, Nature Biotechnology 31, 653-658).

### Target Adjacent Motifs

The IsrB systems disclosed may recognize a target adjacent motif (TAM) in order to recognize and bind a target sequence on a target polynucleotide. In one embodiment, the nucleic acid-guided nucleases and related compositions do not contain a TAM requirement., The precise sequence and length requirements for the TAM will differ depending on the nucleic acid-guided nucleases used. In some examples, TAMs are typically 2-5 base pair sequences adjacent the protospacer (that is, the target sequence). In one example embodiment, the TAM is 3′ adjacent to the target polynucleotide. In another example embodiment, the TAM is 5′ adjacent to the target sequence of the target polynucleotide.

In one embodiment, the cleavage site is distant from the Target Adjacent Motif (TAM), e.g., the cleavage occurs after the nth nucleotide on the non-target strand and after the nucleotide on the targeted strand. In one embodiment, the cleavage site occurs after an identified nucleotide (counted from the TAM) on the non-target strand and after the further identified nucleotide (counted from the TAM) on the targeted strand. In one embodiment, a vector encodes a nucleic acid-targeting effector protein that may be mutated with respect to a corresponding wild-type enzyme such that the mutated nucleic acid-targeting effector protein lacks the ability to cleave one or both DNA and RNA strands of a target polynucleotide containing a target sequence.

TAM identification and specificity may be identified, for example, using the methods disclosed in the Examples section below.

### Paired IsrB Nickases

In one embodiment, the IsrB polypeptide nickase is used in combination with an orthogonal catalytically inactive IsrB polypeptide nuclease to increase efficiency of said nickase (e.g., as described in Chen et al. 2017, Nature Communications 8:14958; doi:10.1038/ncomms14958). More particularly, the orthogonal catalytically inactive IsrB polypeptide nuclease is characterized by a different TAM recognition site than the IsrB nickase used in the AD-functionalized composition and the corresponding guide sequence is selected to bind to a target sequence proximal to that of the nickase of the functionalized IsrB polypeptide nuclease. The orthogonal catalytically inactive IsrB polypeptide nuclease as used in the context of the present invention does not form part of the functionalized composition but merely functions to increase the efficiency of said nickase and is used in combination with a standard hRNA as described in the art for said IsrB polypeptide nuclease. In one embodiment, said orthogonal catalytically inactive IsrB polypeptide nuclease is a dead IsrB polypeptide nuclease, i.e. comprising one or more mutations which abolishes the nuclease activity of said IsrB polypeptide nuclease. In one embodiment, the catalytically inactive orthogonal IsrB polypeptide nuclease is provided with two or more ωRNAs which are capable of hybridizing to target sequences which are proximal to the target sequence of the nickase. In one embodiment, at least two ωRNAs are used to target said catalytically inactive IsrB polypeptide nuclease, of which at least one ωRNA is capable of hybridizing to a target sequence 5″ of the target sequence of the nickase and at least one ωRNA is capable of hybridizing to a target sequence 3′ of the target sequence of the nickase of the functionalized composition, whereby said one or more target sequences may be on the same or the opposite DNA strand as the target sequence of the IsrB polypeptide nickase. In one embodiment, the guide sequences for the one or more ωRNA of the orthogonal catalytically inactive IsrB polypeptide nuclease are selected such that the target sequences are proximal to that of the ωRNA for the targeting of the functionalized composition, e.g. for the targeting of the nickase. In one embodiment, the one or more target sequences of the orthogonal catalytically inactive IsrB polypeptide nuclease are each separated from the target sequence of the nickase by more than 5 but less than 450 basepairs. Optimal distances between the target sequences of the guides for use with the orthogonal catalytically inactive IsrB polypeptide nuclease and the target sequence of the functionalized composition can be determined by the skilled person. In one embodiment, the catalytically inactive orthogonal IsrB polypeptide nuclease has been modified to alter its TAM specificity as described elsewhere herein. In one embodiment, the IsrB polypeptide nickase is a nickase which, by itself has limited activity in human cells, but which, in combination with an inactive orthogonal IsrB polypeptide nuclease and one or more corresponding proximal guides ensures the required nickase activity.

### Methods of Modifying Target Polynucleotides

In one aspect, the present disclosure provides nucleic acid-targeting systems. Such systems may be used to target, modify, and otherwise manipulate a target polynucleotide. In one embodiment, the systems comprise the IsrB polypeptide and one or more ωRNAs. The IsrB polypeptide may have nuclease activity, e.g., capable of cleaving DNA. In on example embodiment, the IsrB polypeptide nuclease may have nickase activity, e.g., capable of generating a single-strand break on a target polynucleotide. IsrB nickases may be used as paired nickases to generated double-strand breaks on target polynucleotides, such as as dsDNA. The IsrB polypeptide nuclease may be in a catalytically dead form.

In one embodiment, formation of a nucleic acid-targeting complex (comprising a guide RNA hybridized to a target sequence and complexed with one or more nucleic acid-targeting effector proteins) results in cleavage of one or both nucleic acid strands in or near (e.g., within 1, 2, 3, 4, 5, 6, 7, 8, 9, 10, 20, 50, or more base pairs from) the target sequence. In one embodiment, one or more vectors driving expression of one or more elements of a nucleic acid-targeting system are introduced into a host cell such that expression of the elements of the nucleic acid-targeting system direct formation of a nucleic acid-targeting complex at one or more target sites. For example, a nucleic acid-targeting effector protein and a ωRNA could each be operably linked to separate regulatory elements on separate vectors. Alternatively, two or more of the elements expressed from the same or different regulatory elements, may be combined in a single vector, with one or more additional vectors providing any components of the nucleic acid-targeting system not included in the first vector. nucleic acid-targeting system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In one embodiment, a single promoter drives expression of a transcript encoding a nucleic acid-targeting effector protein and a ωRNA embedded within one or more intron sequences (e.g. each in a different intron, two or more in at least one intron, or all in a single intron). In one embodiment, the nucleic acid-targeting effector protein and guide RNA are operably linked to and expressed from the same promoter.

### Multiplexing

In one embodiment, IsrB polypeptide may be used in a multiplex (tandem) targeting approach. For example, IsrB polypeptide nuclease herein can employ more than one ωRNA without losing activity. This may enable the use of the IsrB polypeptide nuclease, systems or complexes as defined herein for targeting multiple DNA targets, genes or gene loci, with a single enzyme, system or complex as defined herein. The ωRNA may be tandemly arranged, optionally separated by a nucleotide sequence such as a conserved nucleotide sequence as defined herein. The position of the different ωRNA is the tandem does not influence the activity.

In one aspect, the IsrB polypeptide nucleases may be used for tandem or multiplex targeting. It is to be understood that any of the IsrB polypeptide nucleases, complexes, or compositions herein elsewhere may be used in such an approach. Any of the methods, products, compositions and uses as described herein elsewhere are equally applicable with the multiplex or tandem targeting approach further detailed below. By means of further guidance, the following particular aspects and embodiments are provided.

In one aspect, the invention provides for the use of a IsrB polypeptide nuclease, complex or system as defined herein for targeting multiple gene loci. In one embodiment, this can be established by using multiple (tandem or multiplex) ωRNA sequences.

In one aspect, the invention provides methods for using one or more elements of a IsrB polypeptide nuclease, complex or system as defined herein for tandem or multiplex targeting, wherein said system herein comprises multiple ωRNA sequences. Said ωRNA sequences are separated by a nucleotide sequence, such as a conserved nucleotide sequence as defined herein elsewhere.

The IsrB polypeptide nucleases, compositions, systems or complexes as defined herein provides an effective means for modifying multiple target polynucleotides. The IsrB polypeptide nuclease, system or complex as defined herein has a wide variety of utility including modifying (e.g., deleting, inserting, translocating, inactivating, activating) one or more target polynucleotides in a multiplicity of cell types. As such the IsrB polypeptide nuclease, system or complex as defined herein of the invention has a broad spectrum of applications in, e.g., gene therapy, drug screening, disease diagnosis, and prognosis, including targeting multiple gene loci within a single system.

In one aspect, the present disclosure provides a IsrB polypeptide nuclease, system or complex as defined herein, having a IsrB polypeptide nuclease having at least one destabilization domain associated therewith, and multiple ωRNAs that target multiple nucleic acid molecules such as DNA molecules, whereby each of said multiple ωRNAs specifically targets its corresponding nucleic acid molecule, e.g., DNA molecule. Each nucleic acid molecule target, e.g., DNA molecule can encode a gene product or encompass a gene locus. Using multiple ωRNA hence enables the targeting of multiple gene loci or multiple genes. In one embodiment the IsrB polypeptide nuclease may cleave the DNA molecule encoding the gene product. In one embodiment expression of the gene product is altered. The IsrB polypeptide nuclease and the ωRNAs do not naturally occur together. The present disclosure comprehends the ωRNA comprising tandemly arranged guide sequences. The present disclosure further comprehends coding sequences for the IsrB polypeptide nuclease being codon optimized for expression in a eukaryotic cell. In an embodiment the eukaryotic cell is a mammalian cell, a plant cell or a yeast cell and in a more preferred embodiment the mammalian cell is a human cell. Expression of the gene product may be decreased. The IsrB polypeptide nuclease may form part of a system or complex, which further comprises tandemly arranged ωRNA comprising a series of 2, 3, 4, 5, 6, 7, 8, 9, 10, 15, 25, 25, 30, or more than 30 guide sequences, each capable of specifically hybridizing to a target sequence in a genomic locus of interest in a cell. In one embodiment, the functional system or complex binds to the multiple target sequences. In one embodiment, the functional system or complex may edit the multiple target sequences, e.g., the target sequences may comprise a genomic locus, and In one embodiment, there may be an alteration of gene expression. In one embodiment, the functional system or complex may comprise further functional domains. In one embodiment, the invention provides a method for altering or modifying expression of multiple gene products. The method may comprise introducing into a cell containing said target nucleic acids, e.g., DNA molecules, or containing and expressing target nucleic acid, e.g., DNA molecules; for instance, the target nucleic acids may encode gene products or provide for expression of gene products (e.g., regulatory sequences).

In one embodiment, the IsrB polypeptide nuclease used for multiplex targeting is IsrB with one or more functional domains. In some more specific embodiments, the IscB polypeptide nuclease used for multiplex targeting is a dead IsrB polypeptide nuclease. The inventors have found that the IsrB polypeptide nuclease as described herein may enable improved and/or direct access to one or more nucleotides involved in the DNA: RNA duplex.

### Homologous Recombination Donor Templated Editing

In one embodiment, the compositions and systems herein may comprise one or more nucleic acid templates. In some cases, the nucleic acid template may comprise one or more polynucleotides. In certain cases, the nucleic acid template may comprise coding sequences for one or more polynucleotides. The nucleic acid template may be an RNA template. The nucleic acid template may be a DNA template.

The donor polynucleotide may be used for editing the target polynucleotide. In some cases, the donor polynucleotide comprises one or more mutations to be introduced into the target polynucleotide. Examples of such mutations include substitutions, deletions, insertions, or a combination thereof. The mutations may cause a shift in an open reading frame on the target polynucleotide. In some cases, the donor polynucleotide alters a stop codon in the target polynucleotide. For example, the donor polynucleotide may correct a premature stop codon. The correction may be achieved by deleting the stop codon or introduces one or more mutations to the stop codon. In other example embodiments, the donor polynucleotide addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment refers to less than the entire copy of a gene by providing sufficient nucleotide sequence to restore the functionality of a wild type gene or non-coding regulatory sequence (e.g., sequences encoding long non-coding RNA). In certain example embodiments, the systems disclosed herein may be used to replace a single allele of a defective gene or defective fragment thereof. In another example embodiment, the systems disclosed herein may be used to replace both alleles of a defective gene or defective gene fragment. A “defective gene” or “defective gene fragment” is a gene or portion of a gene that when expressed fails to generate a functioning protein or non-coding RNA with functionality of a corresponding wild-type gene. In certain example embodiments, these defective genes may be associated with one or more disease phenotypes. In certain example embodiments, the defective gene or gene fragment is not replaced but the systems described herein are used to insert donor polynucleotides that encode gene or gene fragments that compensate for or override defective gene expression such that cell phenotypes associated with defective gene expression are eliminated or changed to a different or desired cellular phenotype.

In an embodiment of the invention, the donor polynucleotide may include, but not be limited to, genes or gene fragments, encoding proteins or RNA transcripts to be expressed, regulatory elements, repair templates, and the like. According to the invention, the donor polynucleotides may comprise left end and right end sequence elements that function with transposition components that mediate insertion.

In certain cases, the donor polynucleotide manipulates a splicing site on the target polynucleotide. In some examples, the donor polynucleotide disrupts a splicing site. The disruption may be achieved by inserting the polynucleotide to a splicing site and/or introducing one or more mutations to the splicing site. In certain examples, the donor polynucleotide may restore a splicing site. For example, the polynucleotide may comprise a splicing site sequence.

The donor polynucleotide to be inserted may has a size from 10 basepair or nucleotides to 50 kb in length, e.g., from 50 to 40 k, from 100 and 30 k, from 100 to 10000, from 100 to 300, from 200 to 400, from 300 to 500, from 400 to 600, from 500 to 700, from 600 to 800, from 700 to 900, from 800 to 1000, from 900 to from 1100, from 1000 to 1200, from 1100 to 1300, from 1200 to 1400, from 1300 to 1500, from 1400 to 1600, from 1500 to 1700, from 600 to 1800, from 1700 to 1900, from 1800 to 2000 base pairs (bp) or nucleotides in length.

### Inducible Systems

In one embodiment, a IsrB polypeptide nuclease may form a component of an inducible system. The inducible nature of the system would allow for spatiotemporal control of gene editing or gene expression using a form of energy. The form of energy may include but is not limited to electromagnetic radiation, sound energy, chemical energy and thermal energy. Examples of inducible system include tetracycline inducible promoters (Tet-On or Tet-Off), small molecule two-hybrid transcription activations systems (FKBP, ABA, etc.), or light inducible systems (Phytochrome, LOV domains, or cryptochrome). In one embodiment, the IscB polypeptide or CRISPR-associated IscB polypeptide nuclease may be a part of a Light Inducible Transcriptional Effector (LITE) to direct changes in transcriptional activity in a sequence-specific manner. The components of a light may include a IsrB polypeptide nuclease, a light-responsive cytochrome heterodimer (e.g. from Arabidopsis thaliana), and a transcriptional activation/repression domain. Further examples of inducible DNA binding proteins and methods for their use are provided in U.S. Provisional Application Nos. 61/736,465 and U.S. 61/721,283, and International Patent Publication No. WO 2014/018423 A2 which is hereby incorporated by reference in its entirety.

### Self-Inactivating Systems

Once all copies of a gene in the genome of a cell have been edited, continued expression of the system in that cell is no longer necessary. Indeed, sustained expression would be undesirable in case of off-target effects at unintended genomic sites, etc. Thus time-limited expression would be useful. Inducible expression offers one approach, but in addition Applicants have engineered a self-inactivating system that relies on the use of a non-coding guide target sequence within the vector itself. Thus, after expression begins, the system will lead to its own destruction, but before destruction is complete it will have time to edit the genomic copies of the target gene (which, with a normal point mutation in a diploid cell, requires at most two edits). Simply, the self-inactivating system includes additional RNA (e.g., ωRNA) that targets the coding sequence for the IsrB polypeptide nuclease itself or that targets one or more non-coding guide target sequences complementary to unique sequences present in one or more of the following: (a) within the promoter driving expression of the non-coding RNA elements, (b) within the promoter driving expression of the IsrB polypeptide nuclease gene, (c) within 100 bp of the ATG translational start codon in the IsrB polypeptide nuclease coding sequence, (d) within the inverted terminal repeat (iTR) of a viral delivery vector, e.g., in the AAV genome.

In some aspects, a single ωRNA is provided that is capable of hybridization to a sequence downstream of a IsrB polypeptide nuclease start codon, whereby after a period of time there is a loss of the IsrB polypeptide nuclease expression. In some aspects, one or more ωRNA are provided that are capable of hybridization to one or more coding or non-coding regions of the polynucleotide encoding the system, whereby after a period of time there is a inactivation of one or more, or in some cases all, of the system. In some aspects of the system, and not to be limited by theory, the cell may comprise a plurality of complexes, wherein a first subset of complexes comprise a first @ RNA capable of targeting a genomic locus or loci to be edited, and a second subset of complexes comprise at least one ωRNA capable of targeting the polynucleotide encoding the system, wherein the first subset of complexes mediate editing of the targeted genomic locus or loci and the second subset of complexes eventually inactivate the system, thereby inactivating further expression in the cell.

The various coding sequences (IsrB polypeptide nuclease and ωRNAs) can be included on a single vector or on multiple vectors. For instance, it is possible to encode the enzyme on one vector and the various RNA sequences on another vector, or to encode the enzyme and one ωRNA on one vector, and the remaining ωRNA on another vector, or any other permutation. In general, a system using a total of one or two different vectors is preferred.

Where multiple vectors are used, it is possible to deliver them in unequal numbers, and ideally with an excess of a vector which encodes the first ωRNA relative to the second ωRNA, thereby assisting in delaying final inactivation of the system until genome editing has had a chance to occur.

The first ωRNA can target any target sequence of interest within a genome, as described elsewhere herein. The second ωRNA targets a sequence within the vector which encodes the IsrB polypeptide nuclease, and thereby inactivates the enzyme's expression from that vector. Thus the target sequence in the vector must be capable of inactivating expression. Suitable target sequences can be, for instance, near to or within the translational start codon for the IsrB polypeptide nuclease coding sequence, in a non-coding sequence in the promoter driving expression of the non-coding RNA elements, within the promoter driving expression of the IsrB polypeptide nuclease gene, within 100 bp of the ATG translational start codon in the IscB polypeptide nuclease coding sequence, and/or within the inverted terminal repeat (iTR) of a viral delivery vector, e.g., in the AAV genome. A double stranded break near this region can induce a frame shift in the IsrB polypeptide nuclease coding sequence, causing a loss of protein expression. An alternative target sequence for the “self-inactivating” ωRNA would aim to edit/inactivate regulatory regions/sequences needed for the expression of the system or for the stability of the vector. For instance, if the promoter for the IsrB polypeptide nuclease coding sequence is disrupted then transcription can be inhibited or prevented. Similarly, if a vector includes sequences for replication, maintenance or stability then it is possible to target these. For instance, in a AAV vector a useful target sequence is within the iTR. Other useful sequences to target can be promoter sequences, polyadenylation sites, etc.

Furthermore, if the ωRNA are expressed in array format, the “self-inactivating” ωRNA that target both promoters simultaneously will result in the excision of the intervening nucleotides from within the IsrB polypeptide nuclease expression construct, effectively leading to its complete inactivation. Similarly, excision of the intervening nucleotides will result where the ωRNA target both ITRs, or targets two or more other components simultaneously. Self-inactivation as explained herein is applicable, in general, with systems in order to provide regulation of the systems. For example, self-inactivation as explained herein may be applied to the repair of mutations, for example expansion disorders, as explained herein. As a result of this self-inactivation, repair may be only transiently active.

Addition of non-targeting nucleotides to the 5′ end (e.g. 1-10 nucleotides, preferably 1-5 nucleotides) of the “self-inactivating” ωRNA can be used to delay its processing and/or modify its efficiency as a means of ensuring editing at the targeted genomic locus prior to shut down.

In one aspect of the self-inactivating AAV system, plasmids that co-express one or more ωRNA targeting genomic sequences of interest (e.g. 1-2, 1-5, 1-10, 1-15, 1-20, 1-30) may be established with “self-inactivating” ωRNA that target an IsrB polypeptide nuclease sequence at or near the engineered ATG start site (e.g. within 5 nucleotides, within 15 nucleotides, within 30 nucleotides, within 50 nucleotides, within 100 nucleotides). A regulatory sequence in the U6 promoter region can also be targeted with an ωRNA. The U6-driven guide RNAs may be designed in an array format such that multiple ωRNA sequences can be simultaneously released. When first delivered into target tissue/cells (left cell) ωRNA begin to accumulate while IsrB polypeptide nuclease levels rise in the nucleus. IsrB polypeptide nuclease complexes with all of the ωRNAs to mediate genome editing and self-inactivation of the IsrB polypeptide nuclease plasmids.

One aspect of a self-inactivating system is expression of singly or in tandem array format from 1 up to 4 or more different ωRNA sequences; e.g. up to about 20 or about 30 ωRNA sequences. Each individual self-inactivating ωRNA sequence may target a different target. Such may be processed from, e.g. one chimeric pol3 transcript. Pol3 promoters such as U6 or HI promoters may be used. Pol2 promoters such as those mentioned throughout herein. Inverted terminal repeat (iTR) sequences may flank the Pol3 promoter-ωRNA-Pol2 promoter-IsrB polypeptide nuclease.

One aspect of a tandem array transcript is that one or more ωRNA edit the one or more target(s) while one or more self-inactivating ωRNA inactivate the system. Thus, for example, the described system for repairing expansion disorders may be directly combined with the self-inactivating system described herein. Such a system may, for example, have two ωRNA directed to the target region for repair as well as at least a third ωRNA directed to self-inactivation of the IsrB polypeptide nuclease or systems.

The ωRNA may be a control guide. For example, it may be engineered to target a nucleic acid sequence encoding the IsrB polypeptide nuclease itself, as described in U.S. Patent Publication No. US2015232881A1, the disclosure of which is hereby incorporated by reference. In one embodiment, a system or composition may be provided with just the ωRNA engineered to target the nucleic acid sequence encoding the IsrB polypeptide nuclease. In addition, the system or composition may be provided with the ωRNA engineered to target the nucleic acid sequence encoding the IsrB polypeptide nuclease, as well as nucleic acid sequence encoding the IsrB polypeptide nuclease and, optionally a second @ RNA and, further optionally, a repair template. The second ωRNA may be the primary target of the system or composition (such a therapeutic, diagnostic, knock out etc. as defined herein). In this way, the system or composition is self-inactivating. This is exemplified in relation to Cas in US2015232881A1 (also published as WO2015070083 (A1) referenced elsewhere herein, and may be extrapolated to other IsrB polypeptide nuclease, e.g. IsrB polypeptides.

### Base Editing

The present disclosure also provides for base editing systems. In general, such a system may comprise a deaminase (e.g., an adenosine deaminase or cytidine deaminase) associated (e.g., fused) with a IsrB polypeptide. The IsrB polypeptide may be a dead IsrB polypeptide (such as a IsrB polypeptide nickase, e.g., engineered from a IsrB polypeptide nuclease). In certain examples, the nucleotide deaminase is a mutated form of an adenosine deaminase. The mutated form of the adenosine deaminase may have both adenosine deaminase and cytidine deaminase activities.

In some examples, the present disclosure provides an engineered, non-naturally occurring composition comprising: the nuclei acid-guided nuclease that is catalytically inactive, a nucleotide deaminase associated with or otherwise capable of forming a complex with the IsrB protein, and a single hRNA molecule or single guide RNA molecule capable of forming a complex with the IsrB protein and directing site-specific binding at a target sequence.

In one aspect, the present disclosure provides an engineered adenosine deaminase. The engineered adenosine deaminase may comprise one or more mutations herein. In one embodiment, the engineered adenosine deaminase has cytidine deaminase activity. In certain examples, the engineered adenosine deaminase has both cytidine deaminase activity and adenosine deaminase. In some cases, the modifications by base editors herein may be used for targeting post-translational signaling or catalysis. In one embodiment, compositions herein comprise nucleotide sequence comprising encoding sequences for one or more components of a base editing system. A base-editing system may comprise a deaminase (e.g., an adenosine deaminase or cytidine deaminase) fused with a IsrB polypeptide nuclease or a variant thereof. In some cases, the target polynucleotide is edited at one or more bases to introduce a G→A or C→T mutation.

In some cases, the adenosine deaminase is double-stranded RNA-specific adenosine deaminase (ADAR). Examples of ADARs include those described Yiannis A Savva et al., The ADAR protein family, Genome Biol. 2012; 13 (12): 252, which is incorporated by reference in its entirety. In some examples, the ADAR may be hADAR1. In certain examples, the ADAR may be hADAR2. The sequence of hADAR2 may be that described under Accession No. AF525422.1.

In some cases, the deaminase may be a deaminase domain, e.g., a deaminase domain of ADAR (“ADAR-D”). In one example, the deaminase may be the deaminase domain of hADAR2 (“hADAR2-D), e.g., as described in Phelps K J et al., Recognition of duplex RNA by the deaminase domain of the RNA editing enzyme ADAR2. Nucleic Acids Res. 2015 January; 43 (2): 1123-32, which is incorporated by reference herein in its entirety. In a particular example, the hADAR2-D has a sequence comprising amino acid 299-701 of hADAR2-D, e.g., amino acid 299-701 of the sequence under Accession No. AF525422.1.

In certain examples, the system comprises a mutated form of an adenosine deaminase fused with a dead IsrB polypeptide nuclease (e.g., a IsrB polypeptide nickase). The mutated form of the adenosine deaminase may have both adenosine deaminase and cytidine deaminase activities. In one embodiment, the adenosine deaminase may comprise one or more of the mutations: E488Q based on amino acid sequence positions of hADAR2-D, and mutations in a homologous ADAR protein corresponding to the above. In one embodiment, the adenosine deaminase may comprise one or more of the mutations: E488Q, V351G, based on amino acid sequence positions of hADAR2-D, and mutations in a homologous ADAR protein corresponding to the above. In one embodiment, the adenosine deaminase may comprise one or more of the mutations: E488Q, V351G, S486A, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, based on amino acid sequence positions of hADAR2-D, and mutations in a homologous ADAR protein corresponding to the above. In one embodiment, the adenosine deaminase may comprise one or more of the mutations:

E488Q, V351G, S486A, T375S, S370C, P462A, N597I, L332I, I398V, K350I, M383L, D619G, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, D619G, S582T, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, D619G, S582T, V440I based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, D619G, S582T, V440I, S495N based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, D619G, S582T, V440I, S495N, K418E based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, I398V, K350I, M383L, D619G, S582T, V440I, S495N, K418E, S661T based on amino acid sequence positions of hADAR2-D, and mutations in a homologous ADAR protein corresponding to the above. In some examples, provided herein includes a mutated adenosine deaminase e.g., an adenosine deaminase comprising one or more mutations of E488Q, V351G, S486A, T375S, S370C, P462A, N597I, L332I, I398V, K350I, M383L, D619G, S582T, V440I, S495N, K418E, S661T, fused with a dead IsrB polypeptide nuclease or IsrB polypeptide nickase. In some examples, provided herein includes a mutated adenosine deaminase e.g., an adenosine deaminase comprising E488Q, V351G, S486A, T375S, S370C, P462A, N597I, L332I, I398V, K350I, M383L, D619G, S582T, V440I, S495N, K418E, and S661T, fused with a dead IsrB polypeptide nuclease or IsrB polypeptide nickase. In some examples, provided herein includes a mutated adenosine deaminase e.g., an adenosine deaminase comprising E488Q, V351G, S486A, T375S, S370C, P462A, N597I, L332I, I398V, K350I, M383L, D619G, S582T, V440I, S495N, K418E, S661T, and S375N fused with a dead IsrB polypeptide nuclease or IsrB polypeptide nickase.

In one embodiment, the adenosine deaminase may be a tRNA-specific adenosine deaminase or a variant thereof. In one embodiment, the adenosine deaminase may comprise one or more of the mutations: W23L, W23R, R26G, H36L, N37S, P48S, P48T, P48A, 149V, R51L, N72D, L84F, S97C, A106V, D108N, H123Y, G125A, A142N, S146C, D147Y, R152H, R152P, E155V, I156F, K157N, K161T, based on amino acid sequence positions of E. coli TadA, and mutations in a homologous deaminase protein corresponding to the above. In one embodiment, the adenosine deaminase may comprise one or more of the mutations: D108N based on amino acid sequence positions of E. coli TadA, and mutations in a homologous deaminase protein corresponding to the above. In one embodiment, the adenosine deaminase may comprise one or more of the mutations: A106V, D108N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, based on amino acid sequence positions of E. coli TadA, and mutations in a homologous deaminase protein corresponding to the above. In one embodiment, the adenosine deaminase may comprise one or more of the mutations: A106V, D108N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, A142N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, A142N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, W23R, P48A, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, W23R, P48A, A142N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, W23R, P48A, R152P, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, W23R, P48A, R152P, A142N, based on amino acid sequence positions of E. coli TadA, and mutations in a homologous deaminase protein corresponding to the above.

In some examples, the base editing systems may comprise an intein-mediated trans-splicing system that enables in vivo delivery of a base editor, e.g., a split-intein cytidine base editors (CBE) or adenine base editor (ABE) engineered to trans-splice. Examples of the such base editing systems include those described in Colin K. W. Lim et al., Treatment of a Mouse Model of ALS by In Vivo Base Editing, Mol Ther. 2020 Jan. 14. pii: S1525-0016 (20) 30011-3. doi: 10.1016/j.ymthe.2020.01.005; and Jonathan M. Levy et al., Cytosine and adenine base editing of the brain, liver, retina, heart and skeletal muscle of mice via adeno-associated viruses, Nature Biomedical Engineering volume 4, pages97-110 (2020), which are incorporated by reference herein in their entireties.

Examples of base editing systems include those described in International Patent Publication Nos. WO 2019/071048 (e.g. paragraphs [0933]-[0938]), WO 2019/084063 (e.g., paragraphs [0173]-[0186], [0323]-[0475], [0893]-[1094]), WO 2019/126716 (e.g., paragraphs [0290]-[0425], [1077]-[1084]), WO 2019/126709 (e.g., paragraphs [0294]-[0453]), WO 2019/126762 (e.g., paragraphs [0309]-[0438]), WO 2019/126774 (e.g., paragraphs [0511]-[0670]), Cox et al., RNA editing with CRISPR-Cas13, 358 Science. 1019-1027 (2017); Abudayyeh et al., A cytosine deaminase for programmable single-base RNA editing, 365 Science 382-386 (July 2019).; Gaudelli et al., Programmable base editing of A·T to G·C in genomic DNA without DNA cleavage, 551 Nature volume 464-471 (November 2017); Komor et al., Programmable editing of a target base in genomic DNA without double-stranded DNA cleavage. 19 Nature. 420-4 (2016); Doman et al., Evaluation and minimization of Cas9-independent off-target DNA editing by cytosine base editors, Nat Biotechnol (2020). doi.org/10.1038/s41587-020-0414-6; and Richter et al., Phage-assisted evolution of an adenine base editor with improved Cas domain compatibility and activity, Nat Biotechnol (2020). doi.org/10.1038/s41587-020-0453-z, which are incorporated by reference herein in their entireties and can be used to adapt to the IsrB polypeptides for base editing purposes.

### Prime Editing

In one embodiment, the present disclosure provides compositions and systems may comprise a catalyticall inactive IsrB polypeptide fused or linked to a reverse transcriptase and a prime editing ωRNA that both specifies the target site and encodes the desired edit. See e.g. Anzalone et al. Search-and-replace genome editing without double-strand breaks or donor DNA. 576 Nature, 149-157 (2019). Prime editing systems may be used to precisely install C⋅G-to-T∩A or A⋅T-to-G⋅C transition mutations, or C⋅G-to-G⋅C transversion mutations without requiring single or double-strand breaks. Prime editors can install any of the twelve possible base pair substitutions as well as small insertion and deletions (e.g. up to ˜40 bp insertions and ˜80 bp deletions). In addition, twin prime editing, which enables the deletion, substitution, or insertion of larger DNA sequences may be used. See e.g. Twin prime editing uses a pair of prime editing ωRNAs, each of which targets a different DNA strand around a targeted modification site on a target polynucleotide and templates the synthesis of a 3′flap that is complementary to the 3′ flap templated by the other prime editing ωRNA. See e.g. Anzalone et al. Programmable large DNA deletion, replacement, integration, and inversion with twin prime editing and site-specific recombinases. bioRxiv (November (2, 2021), https://doi.org/10.1101/2021/11.01.466780.

In certain aspects, the reverse transcriptase is Human immunodeficiency virus (HIV) RT, Avian myoblastosis virus (AMV) RT, Moloney murine leukemia virus (M-MLV) RT a group II intron RT, a group II intron-like RT, or a chimeric RT. In an embodiment, the RT comprises modified forms of these RTs, such as, engineered variants of Avian myoblastosis virus (AMV) RT, Moloney murine leukemia virus (M-MLV) RT, or Human immunodeficiency virus (HIV) RT (see, e.g., Anzalone, et al., Search-and-replace genome editing without double-strand breaks or donor DNA, Nature. 2019 December; 576 (7785): 149-157).

In some examples, the compositions and systems may comprise the IsrB protein disclosed herein; a reverse transcriptase (RT) polypeptide connected to or otherwise capable of forming a complex with the IsrB polypeptide; and a ωRNA capable of forming a complex with the IsrB polypeptide and comprising: a ωRNA capable of directing site-specific binding of the IsrB polypeptide complex to a target sequence of a target polynucleotide; a 3′ binding site region capable of binding to a cleaved upstream strand of the target polynucleotide; and a RT template sequence encoding an extended sequence, wherein the extended sequence comprises a variant region and a 3′ homologous sequence capable of hybridization to the downstream cleaved strand of the target polynucleotide.

A reverse transcriptase domain may be a reverse transcriptase or a fragment thereof. A wide variety of reverse transcriptases (RT) may be used in alternative embodiments of the present invention, including prokaryotic and eukaryotic RT, provided that the RT functions within the host to generate a donor polynucleotide sequence from the RNA template. If desired, the nucleotide sequence of a native RT may be modified, for example using known codon optimization techniques, so that expression within the desired host is optimized. A reverse transcriptase (RT) is an enzyme used to generate complementary DNA (cDNA) from an RNA template, a process termed reverse transcription. Reverse transcriptases are used by retroviruses to replicate their genomes, by retrotransposon mobile genetic elements to proliferate within the host genome, by eukaryotic cells to extend the telomeres at the ends of their linear chromosomes, and by some non-retroviruses such as the hepatitis B virus, a member of the Hepadnaviridae, which are dsDNA-RT viruses. Retroviral RT has three sequential biochemical activities: RNA-dependent DNA polymerase activity, ribonuclease H, and DNA-dependent DNA polymerase activity. Collectively, these activities enable the enzyme to convert single-stranded RNA into double-stranded cDNA. In an embodiment, the RT domain of a reverse transcriptase is used in the present invention. The domain may include only the RNA-dependent DNA polymerase activity. In some examples, the RT domain is non-mutagenic, i.e., does not cause mutation in the donor polynucleotide (e.g., during the reverse transcriptase process). In some cases, In some examples, the RT domain may be non-retron RT, e.g., a viral RT or a human endogenous RTs. In some examples, the RT domain may be retron RT or DGRs RT. In some example, the RT may be less mutagenic than a counterpart wildtype RT. In one embodiment, the RT herein is not mutagenic.

The reverse transcriptase may be fused to the C-terminus of a IsrB polypeptide nuclease. Alternatively or additionally, the reverse transcriptase may be fused to the N-terminus of a IsrB polypeptide nuclease. The fusion may be via a linker and/or an adaptor protein. In some examples, the reverse transcriptase may be an M-MLV reverse transcriptase or variant thereof. The M-MLV reverse transcriptase variant may comprise one or more mutations. For the examples, the M-ML V reverse transcriptase may comprise D200N, L603W, and T330P. In another example, the M-MLV reverse transcriptase may comprise D200N, L603W, T330P, T306K, and W313F. In a particular example, the fusion of IsrB polypeptide nuclease and reverse transcriptase is IsrB polypeptide nuclease (with a mutation corresponding to H840A of SpCas9) reverse fused with M-MLV transcriptase (D200N+L603W+T330P+T306K+W313F).

A single-strand break (a nick) may be generated on the target DNA by the IsrB polypeptide nuclease at the target site to expose a 3′-hydroxyl group, thus priming the reverse transcription of an edit-encoding extension on the ωRNA directly into the target site. These steps may result in a branched intermediate with two redundant single-stranded DNA flaps: a 5′ flap that contains the unedited DNA sequence, and a 3′ flap that contains the edited sequence copied from the hRNA. The 5′ flaps may be removed by a structure-specific endonuclease, e.g., FEN122, which excises 5′ flaps generated during lagging-strand DNA synthesis and long-patch base excision repair. The non-edited DNA strand may be nicked to induce bias DNA repair to preferentially replace the non-edited strand. Examples of prime editing systems and methods include those described in Anzalone A V et al., Search-and-replace genome editing without double-strand breaks or donor DNA, Nature. 2019 Oct. 21. doi: 10.1038/s41586-019-1711-4, which is incorporated by reference herein in its entirety.

The IsrB polypeptide nuclease may be used to prime-edit a single nucleotide on a target DNA. Alternatively or additionally, the IsrB polypeptide nuclease may be used to prime-edit at least 2, at least 3, at least 4, at least 5, at least 6, at least 7, at least 8, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 200, at least 300, at least 400, at least 500, at least 600, at least 700, at least 800, at least 900, or at least 1000 nucleotides on a target DNA.

In yet another embodiment, PRIME editing is used first to create a longer 3′ region (e.g. 20 nucleotides). Examples of prime editing systems and methods include those described in Anzalone A V et al., Search-and-replace genome editing without double-strand breaks or donor DNA, Nature. 2019 Oct. 21. doi: 10.1038/s41586-019-1711-4, which is incorporated by reference herein in its entirety. In such cases, the system comprises a IsrB polypeptide with nickase activity, a reverse transcriptase domain, and a DNA polymerase, and a ωRNA comprising a binding sequence capable of hybridizing to the target polynucleotide and a editing sequence. The generated region may be further extended on a DNA template as described herein. The latter may allow generation of a target-independent sequence, compatible with a generic donor sequence.

In some variations, a second IsrB polypeptide-mediated cleavage in vicinity to the target site may be made, which may enable more efficient invasion of the extended DNA.

In some examples, the compositions and systems of the IsrB polypeptide herein comprise: a reverse transcriptase (RT) polypeptide connected to or otherwise capable of forming a complex with the IsrB polypeptide; a first ωRNA capable of forming a first IsrB polypeptide-Reverse transcriptase complex with the IsrB polypeptide and comprising: a ωRNA capable of directing site-specific binding of the first IscB polypeptide-Reverse transcriptase complex to a first target sequence of a target polynucleotide; a first binding site region capable of binding to a cleaved or nicked strand of the target polynucleotide; and a RT template sequence encoding a first extended sequence; a second RNA capable of forming a second IsrB polypeptide-Reverse transcriptase complex with the IsrB polypeptide and comprising: a ωRNA capable of directing site specific binding of the second IsrB polypeptide-Reverse transcriptase complex to a second target sequence of the target polynucleotide; a second binding site region capable of binding to a cleaved or nicked strand of the target polynucleotide; and a RT template sequence encoding a second extended sequence.

In some cases, the compositions and systems may further comprise: a donor template; a third @ RNA capable of forming a IsrB polypeptide-Reverse transcriptase complex-ωRNA with the IsrB polypeptide and comprising: a ωRNA sequence capable of directing site-specific binding to a target sequence on the donor template; a third binding region capable of binding to a cleaved or nicked strand of the donor template; and a RT template encoding a third extended region complementary to the first extended region generated on the target polynucleotide; and a fourth ωRNA sequence capable of forming a IsrB polypeptide-Reverse transcriptase complex with the IsrB polypeptide and comprising: a ωRNA sequence capable of directing site-specific binding to a second target sequence on the donor template; a fourth binding region capable of binding to a cleaved or nicked strand of the donor template; and a RT template encoding a fourth extended region complementary to the second extended region generated on the target polynucleotide.

In some cases, the compositions and systems may further comprise a site-specific recombinase, and wherein the first and second extended regions are complementary to each other and introduce a serine integrase recombination site; and a donor molecule comprising a donor sequence for insertion into the target polypeptide and the complementary recombination site to the serine integrase recombination site.

In some examples, the compositions and systems may further comprise a recombinase. The recombinase is connected to or otherwise capable of forming a complex with the IsrB polypeptide. In an embodiment, the complex is capable of inserting a recombination site in the DNA loci of interest by extension of RT templates that encode for the recombination site on the 3′ extension of the @ RNA sequences by the reverse transcriptase. In an embodiment, a donor template comprising a compatible recombination site is provided that can recombine unidirectionally with the inserted recombination site when a recombinase specific for the recombination site is also provided. In an embodiment, the donor template is a plasmid comprising the complementary recombination site and any sequence for insertion at the DNA loci of interest. In an embodiment, the recombinase is connected to or capable of forming a complex with the IsrB polypeptide, such that all of the enzymatic proteins are brought into contact at the loci of interest. In an embodiment, the recombinase is codon optimized for eukaryotic cells (described further herein). In an embodiment, the recombinase includes a NLS (described further herein). In an embodiment, the recombinase is provided as a separate protein. The separate recombinase may form a dimer and bind to the donor template recombination site. The recombinase may be targeted to the loci of interest as a result of the insertion of the compatible recombination site that is also recognized by the recombinase. Thus, the recombinase may recognize the recombination site inserted at the DNA loci of interest and the recombination site on the donor and be targeted to the DNA loci of interest without any additional modifications to the recombinase.

In an embodiment, a second IsrB complex connected to a recombinase is targeted to the DNA loci of interest. In an embodiment, the second IsrB complex comprises a dead IsrB protein (dIsrB, described further herein), such that the recombinase is targeted to the DNA loci of interest, but the target sequence is not further cleaved. In an embodiment, the dIsrB targets a sequence generated only after the insertion of the recombination site. In an embodiment, the recombinase recognizes and binds to the donor template recombination site and the inserted recombination site. In an embodiment, the recombinase forms a dimer with a recombinase provided as a separate protein.

As used herein, the term “Recombinase” refers to an enzyme that catalyzes recombination between two or more recombination sites (e.g., an acceptor and donor site). Recombinases useful in the present invention catalyze recombination at specific recombination sites which are specific polynucleotide sequences that are recognized by a particular recombinase. “Uni-directional recombinases” or “integrases” refer to recombinase enzymes whose recognition sites are destroyed after the recombination has taken place. The term “integrase” refers to a type of recombinase. In other words, the sequence recognized by the recombinase is changed into one that is not recognized by the recombinase upon recombination. As a result, once a sequence is subjected to recombination by the uni-directional recombinase, the continued presence of the recombinase cannot reverse the previous recombination event.

“Recombination sites” are specific polynucleotide sequences that are recognized by the recombinase enzymes described herein. Typically, two different sites are involved (in regards to recombination termed “complementary sites”), one present in the target nucleic acid (e.g., a chromosome or episome of a eukaryote) and another on the nucleic acid that is to be integrated at the target recombination site. The terms “attB” and “attP,” which refer to attachment (or recombination) sites originally from a bacterial target (attachment site of bacteria) and a phage donor (attachment site of phage), respectively, are used herein although recombination sites for particular enzymes may have different names. The two attachment sites can share as little sequence identity as a few base pairs. The recombination sites typically include left and right arms separated by a core or spacer region. Thus, an attB recombination site consists of BOB′, where B and B′ are the left and right arms, respectively, and O is the core region. Similarly, attP is POP′, where P and P′ are the arms and O is again the core region. Upon recombination between the attB and attP sites, and concomitant integration of a nucleic acid at the target, the recombination sites that flank the integrated DNA are referred to as “attL” and “aatR.” The attL and attR sites, using the terminology above, thus consist of BOP′ and POB′, respectively. In some representations herein, the “O” is omitted and attB and attP, for example, are designated as BB′ and PP′, respectively.

### Guided Transposition Systems

Embodiments disclosed herein provide an engineered or non-natural guided transposition system. The engineered or non-natural guided transposition system may comprise one or more components of a IsrB system and one or more components of a Class II transposon. The components of the @ RNA-IsrB system can direct the Class II transposon component(s) to retrotransposon to a target nucleic acid sequence and direct its transposition into a recipient polynucleotide.

For example, the engineered or non-natural guided transposition systems that can include (a) a first IsrB polypeptide; (b) a first Class II transposon polypeptide coupled to or otherwise capable of complexing with the first IsrB polypeptide; (c) a first guide molecule capable of forming a first ωRNA-IsrB complex with the first IsrB protein and directing site-specific binding to a first target sequence of a first target polynucleotide; (d) a second IsrB polypeptide; (e) a second Class II transposon polypeptide coupled to or otherwise capable of complexing with the second IsrB polypeptide; (f) a second guide molecule capable of forming a second @ RNA-IsrB complex with the first IsrB protein and directing site-specific binding to a second target sequence of the first target polynucleotide; and (g) a Class II transposon polynucleotide comprising the first target polynucleotide and is capable of forming a complex with the first and second IsrB polypeptide, the first and second guide molecules, and the first and second Class II transposon polypeptides.

In one embodiment, the engineered or non-natural guided excision-transposition system can include (h) a third guide molecule capable of complexing with the first IsrB polypeptide and directing site-specific binding to a first target sequence of a second target polynucleotide, wherein the third guide molecule is optionally coupled to the first IsrB polypeptide; (i) optionally, a first ωRNA polynucleotide that encodes the third ωRNA; (j) a fourth ωRNA capable of complexing with the second IsrB polypeptide and directing site-specific binding to a second target sequence of the second target polynucleotide, wherein the fourth guide molecule is optionally coupled to the second IsrB polypeptide; and (k) optionally, a second ωRNA polynucleotide that encodes the fourth ωRNA.

In one embodiment, the first and the second Class II transposon polypeptides are capable of excising the first target polynucleotide from the Class II transposon polynucleotide. In one embodiment, the first and the second Class II transposon polypeptides are capable of transposing the first target polynucleotide in the second target polynucleotide. In one embodiment, the first target polynucleotide does not include one or more Class II transposon long terminal repeats.

The engineered or non-natural guided excision-transposition systems described herein can be based on a Class II transposon or Class II transposon system. The engineered or non-natural guided excision-transposition system may include a first target polynucleotide, also referred to as a donor polynucleotide or transposon and a second target polynucleotide, which is also referred to herein as a recipient polynucleotide. As used herein, “transposon” (also referred to as transposable element) refers to a polynucleotide sequence that is capable of moving form location in a genome to another. There are several classes of transposons. Transposons include retrotransposons (Class I transposons) and DNA transposons (Class II transposons). In some cases, retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide.

Any suitable transposon system can be used. Suitable transposon and systems thereof can include, but are not limited, to Sleeping Beauty transposon system (Tcl/mariner superfamily) (see e.g. Ivics et al. 1997. Cell. 91 (4): 501-510), piggyBac (piggyBac superfamily) (see e.g. Li et al. 2013 110 (25): E2279-E2287 and Yusa et al. 2011. PNAS. 108 (4): 1531-1536), Tol2 (superfamily hAT), Frog Prince (Tcl/mariner superfamily) (see e.g. Miskey et al. 2003 Nucleic Acid Res. 31 (23): 6873-6881) and variants thereof.

In one embodiment, the first and/or second Class II transposon polypeptide is a DD[E/D] transposon or transposon polypeptide. In one embodiment, the first and/or the second Class II transposon polynucleotide is a Tcl/mariner, PiggyBac, Frog Prince, Tn3, Tn5, hAT, CACTA, P, Mutator, PIF/Harbinger, Transib, or a Merlin/IS1016 transposon polynucleotide. In one embodiment, the first and/or second Class II transposon polypeptide is a Tcl/mariner, PiggyBac, Frog Prince, Tn3, Tn5, hAT, CACTA, P, Mutator, PIF/Harbinger, Transib, or a Merlin/IS1016 transposon polypeptide.

Suitable Class II transposon systems and components that can be utilized can also be and are not limited to those described in e.g. and without limitation, Han et al., 2013. BMC Genomics. 14:71, doi: 10.1186/1471-2164-14-71, Lopez and Garcia-Perez. 2010. Curr. Genomics. 11 (2): 115-128; Wessler. 2006. PNAS. 103 (47): 176000-17601; Gao et al., 2017. Marine Genomics. 34:67-77; Bradic et al. 2014. Mobile DNA. 5 (12) doi: 10.1186/1759-8753-5-12; Li et al., 2013. PNAS. 110 (25) E2279-E2287; Kebriaei et al. 2017. Trends in Genetics. 33 (11): 852-870); Miskey et al. 2003. Nucleic Acid res. 31 (23): 6873-6881; Nicolas et al. 2015. Microbiol Spectr. 3 (4) doi: 10.1128/microbiolspec.MDNA3-0060-2014); W.S. Reznikoff. 1993. Annu Rev. Microbiol. 47:945-963; Rubin et al. 2001. Genetics. 158 (3): 949-957; Wicker et al. 2003. Plant Physiol. 132 (1): 52-63; Majumdar and Rio. 2015. Microbiol. Spectr. 3 (2) doi: 10.1128/microbiolspec.MDNA3-0004-2014; D. Lisch. 2002. Trends in Plant Sci. 7 (11): 498-504; Sinzelle et al. 2007. PNAS. 105 (12): 4715-4720; Han et al. 2014; Genome Biol. Evol. 6 (7): 1748-1757; Grzebelus et al. 2006; Mol. Genet. Genomics. 275 (5): 450-459; Zhang et al. 2004. Genetics. 166 (2): 971-986; Chen and Li. 2008. Gene. 408 (1-2): 51-63; and C. Feschotte. 2004. Mol. Biol. Evol. 21 (9): 1769-1780.

### Retrotransposons

The systems and compositions herein may comprise a IsrB polypeptide nuclease, one or more ωRNAs, and one or more components of a retrotransposon, e.g., a non-LTR retrotransposon. The one or more components of a retrotransposon include a retrotransposon protein and retrotransposon RNA. The systems and compositions may be used to insert a donor polynucleotide to a target polynucleotide. The systems and compositions may further comprise a donor polynucleotide.

In some examples, the present disclosure provides an engineered, non-naturally occurring composition comprising: a IsrB polypeptide nuclease, a non-LTR retrotransposon protein associated with or otherwise capable of forming a complex with the IsrB polypeptide nuclease; a single ωRNA capable of forming a complex with the IsrB polypeptide nuclease and directing site-specific binding to a target sequence of a target polynucleotide. The composition may further comprise a donor construct comprising a donor polynucleotide for insertion to the target polynucleotide and located between two binding elements capable of forming a complex with the non-LTR retrotransposon protein. In some cases, the IsrB polypeptide nuclease is engineered to have improved nickase activity.

In some examples, the IsrB polypeptide nuclease is fused to the N-terminus of the non-LTR retrotransposon protein. In some examples, the IsrB polypeptide nuclease is fused to the C-terminus of the non-LTR retrotransposon protein.

The guides may direct the fusion protein to a target sequence 5′ of the targeted insertion site, and wherein the IsrB polypeptide nuclease generates a double-strand break at the targeted insertion site. The guides may direct the fusion protein to a target sequence 3′ of the targeted insertion site, and wherein the IsrB polypeptide nuclease generates a double-strand break at the targeted insertion site.

The donor polynucleotide may further comprise a polymerase processing element to facilitate 3′ end processing of the donor polynucleotide sequence. The polymerase may be a DNA polymerase, e.g., DNA polymerase I. In some examples, the polymerase may be an RNA polymerase.

In some examples, the donor polynucleotide further comprises a homology region to the target sequence on the 5′ end of the donor construct, the 3′ end of the donor construct, or both. In some examples, the homology region is from 1 to 50, from 5 to 30, from 8 to 25, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 base pairs in length.

Native or wild-type non-LTR retrotransposons encode the protein machinery necessary for their self-mobilization. The non-LTR retrotransposon element comprises a DNA element integrated into a host genome. This DNA element may encode one or two open reading frames (ORFs). For example, the R2 element of Bombyx mori encodes a single ORF containing reverse transcriptase (RT) activity and a restriction enzyme-like (REL) domain. L1 elements encode two ORFs, ORF1 and ORF2. ORF1 contains a leucine zipper domain involved in protein-protein interactions and a C-terminal nucleic acid binding domain. ORF2 has a N-terminal apurinic/apyrimidinic endonuclease (APE), a central RT domain, and a C-terminal cysteine histidine rich domain. An example replicative cycle of a non-LTR retrotransposon may comprise transcription of the full-length retrotransposon element to generate an mRNA active element (retrotransposon RNA). The active element mRNA is translated to generate the encoded retrotransposon proteins or polypeptides. A ribonucleoprotein complex comprising the active element and retrotransposon protein or polypeptide is formed and this RNP facilitates integration of the active element into the genome. The RNA-transposase complex nicks the genome. The 3′ end of the nicked DNA serves as a primer to allow the reverse transcription of the transposon RNA into cDNA. Fourth, the transposase proteins integrate the cDNA into the genome.

Elements of these systems may be engineered to work within the context of the invention. For example a non-LTR retrotransposon polypeptide may be fused to a site-specific nuclease. The binding elements that allow a non-LTR retrotransposon polypeptide to bind to the native retrotransposon DNA element, may be engineered into a donor construct to facilitate entry of a donor polynucleotide sequence into a target polypeptide.

In the present invention the protein component of the non-LTR retrotransposon may be connected to or otherwise engineered to form a complex with a site-specific nuclease. The retrotransposon RNA may be engineered to encode a donor polynucleotide sequence. Thus, in certain example embodiments, the IscB polypeptide nuclease, via formation of a IscB polypeptide nuclease complex with a guide sequence, directs the retrotransposon complex (e.g. the retrotransposon polypeptide(s) and retrotransposon RNA to a target sequence in a target polynucleotide, where the retrotransposon RNP complex facilitates integration of the donor polynucleotide sequence into the target polynucleotide. Accordingly, the one or more non-LTR retrotransposon components may comprise retrotransposon polypeptides, or function domains thereof, that facilitate binding of the retrotransposon RNA, reverse transcription of the retrotransposon RNA into cDNA, and/or integration of the donor polynucleotide into the target polynucleotide, as well as retrotransposon RNA elements modified to encode the donor polynucleotide sequence.

Examples of non-LTR retrotransposons include CRE, R2, R4, L1, RTE, Tad, R1, LOA, I, Jockey, CR1. In one example, the non-LTR retrotransposon is R2. In another example, the non-LTR retrotransposon is L1. Examples of non-LTR retrotransposons may include those described in Christensen S M et al., RNA from the 5′ end of the R2 retrotransposon controls R2 protein binding to and cleavage of its DNA target site, Proc Natl Acad Sci USA. 2006 Nov. 21; 103 (47): 17602-7; Eickbush T H et al, Integration, Regulation, and Long-Term Stability of R2 Retrotransposons, Microbiol Spectr. 2015 April; 3 (2): MDNA3-0011-2014. doi: 10.1128/microbiolspec.MDNA3-0011-2014; Han J S, Non-long terminal repeat (non-LTR) retrotransposons: mechanisms, recent developments, and unanswered questions, Mob DNA. 2010 May 12; 1 (1): 15. doi: 10.1186/1759-8753-1-15; Malik H S et al., The age and evolution of non-LTR retrotransposable elements, Mol Biol Evol. 1999 June; 16 (6): 793-805, which are incorporated by reference herein in their entireties.

Examples of the non-LTR retrotransposon polypeptides also include R2 from Clonorchis sinensis, or Zonotrichia albicollis.

A non-LTR retrotransposon may comprise multiple retrotransposon polypeptides or polynucleotides encoding same. In one embodiment, the retrotransposon polypeptides may form a complex. For example, a non-LTR retrotransposon is a dimer, e.g., comprising two retrotransposon polypeptides forming a dimer. The dimer subunits may be connected or form a tandem fusion. A IscB polypeptide nuclease may be associate with (e.g., connected to) one or more subunits of such complex. In some examples, the non-LTR retrotransposon is a dimer of two retrotransposon polypeptides; one of the retrotransposon polypeptides comprises nuclease or nickase activity and is connected with a IscB polypeptide nuclease.

The retrotransposon polypeptides may comprise one or more modifications to, for example, enhance specificity or efficiency of donor polynucleotide recognition, target-primed template recognition (TPTR). The retrotransposon polypeptides may also comprise one or more truncations or excisions to remove domains or regions of wild-type protein to arrive at a minimal polypeptide that retain donor polynucleotide recognition and TPTR. In some example embodiments, the native endonuclease activity may be mutated to eliminate endonuclease activity.

In certain example embodiments, the modifications or truncations of the non-LTR retrotransposon peptide may be in a zinc finger region, a Myb region, a basic region, a reverse transcriptase domain, a cysteine-histidine rich motif, or an endonuclease domain.

A non-LTR retrotransposon may comprise polynucleotide encoding one or more retrotransposon RNA molecules. The polynucleotide may comprise one or more regulatory elements. The regulatory elements may be promoters. The regulatory elements and promoters on the polynucleotides include those described throughout this application. For example, the polynucleotide may comprise a pol2 promoter, a pol3 promoter, or a T7 promoter.

In some cases, the polynucleotide encodes a retrotransposon RNA with at least a portion of its sequence complementary to a target sequence. For example, the 3′ end of the retrotransposon RNA may be complementary to a target sequence. The RNA may be complementary to a portion of a nicked target sequence. In one embodiment, a retrotransposon RNA may comprise one or more donor polynucleotides. In certain cases, a retrotransposon RNA may encode one or more donor polynucleotides.

A retrotransposon RNA may be capable of binding to a retrotransposon polypeptide. Such retrotransposon RNA may comprise one or more elements for binding to the retrotransposon polypeptide. Examples of binding elements include hairpin structures, pseudoknots (e.g., a nucleic acid secondary structure containing at least two stem-loop structures in which half of one stem is intercalated between the two halves of another stem), stem loops, and bulges (e.g., unpaired stretches of nucleotides located within one strand of a nucleic acid duplex). In certain examples, the retrotransposon RNA comprises one or more hairpin structures. In some examples, the retrotransposon RNA comprises one or more pseudoknots. In certain examples, a retrotransposon RNA comprises a sequence encoding a donor polynucleotide and one or more binding elements for forming a complex with the retrotransposon polypeptide. The binding elements may be located on the 5′ end or the 3′ end.

In one embodiment, a retrotransposon RNA comprises a region capable of hybridizing with an overhang of a target polynucleotide at the target site. The overhang may be a stretch of single-stranded DNA. The overhang may function as a primer for reverse transcription of at least a portion of the retrotransposon RNA to a cDNA. In some cases, a region of the cDNA may be capable of hybridizing a second overhang of the target polynucleotide. The second overhang may function as a primer for the synthesis of a second strand to generate a double-stranded cDNA. The cDNA may comprise a donor polynucleotide sequence. The two overhangs may be from different strands of the target polynucleotide.

### Reverse Transcriptase Domain

The one or more functional domains may be one or more reverse transcriptase domains. In one embodiment, the systems comprise an engineered system for modifying a target polynucleotide comprising: a IscB polypeptide or CRISPR-associated IscB polypeptide or a variant thereof (e.g., dIscB); a reverse transcriptase (RT) domain; a RNA template comprising or encoding a donor polynucleotide to be inserted to a target sequence of the target polynucleotide; and an ωRNA or guide RNA molecule (i.e., a naturally single guide RNA molecule comprising a scaffold for reprogramming).

The reverse transcriptase may generate single-strand DNA based on the RNA template. The single-strand DNA may be generated by a non-retron, retron, or diversity generating retroelement (DGR). In some examples, the single-strand DNA may be generated from a self-priming RNA template. A self-priming RNA template may be used to generate a DNA without the need of a separate primer.

A reverse transcriptase domain may be a reverse transcriptase or a fragment thereof. A wide variety of reverse transcriptases (RT) may be used in alternative embodiments of the present invention, including prokaryotic and eukaryotic RT, provided that the RT functions within the host to generate a donor polynucleotide sequence from the RNA template. If desired, the nucleotide sequence of a native RT may be modified, for example using known codon optimization techniques, so that expression within the desired host is optimized. A reverse transcriptase (RT) is an enzyme used to generate complementary DNA (cDNA) from an RNA template, a process termed reverse transcription. Reverse transcriptases are used by retroviruses to replicate their genomes, by retrotransposon mobile genetic elements to proliferate within the host genome, by eukaryotic cells to extend the telomeres at the ends of their linear chromosomes, and by some non-retroviruses such as the hepatitis B virus, a member of the Hepadnaviridae, which are dsDNA-RT viruses. Retroviral RT has three sequential biochemical activities: RNA-dependent DNA polymerase activity, ribonuclease H, and DNA-dependent DNA polymerase activity. Collectively, these activities enable the enzyme to convert single-stranded RNA into double-stranded cDNA. In an embodiment, the RT domain of a reverse transcriptase is used in the present invention. The domain may include only the RNA-dependent DNA polymerase activity. In some examples, the RT domain is non-mutagenic, i.e., does not cause mutation in the donor polynucleotide (e.g., during the reverse transcriptase process). In some examples, the RT domain may be non-retron RT, e.g., a viral RT or a human endogenous RT. In some examples, the RT domain may be retron RT or DGRs RT. In some examples, the RT may be less mutagenic than a counterpart wildtype RT. In one embodiment, the RT herein is not mutagenic.

### Retrons

In an embodiment, a donor template for homologous recombination is generated by use of a self-priming RNA template for reverse transcription. A non-limiting example of a self-priming reverse transcription system is the retron system. By the term “retron” it is meant a genetic element which encodes components enabling the synthesis of branched RNA-linked single stranded DNA (msDNA) and a reverse transcriptase. Retrons which encode msDNA are known in the art, for example, but not limited to U.S. Pat. Nos. 6,017,737; 5,849,563; 5,780,269; 5,436,141; 5,405,775; 5,320,958; CA 2,075,515; all of which are herein incorporated by reference.

In an embodiment, the reverse transcriptase domain is a retron RT domain. In an embodiment, the RNA template encodes a retron RNA template that is recognized and reverse transcribed by the retron reverse transcriptase domain. Conserved across many bacterial species, retrons are highly efficient reverse transcription systems of relatively unknown function. The retron system consists of the retron RT protein, as well as the msr and msd transcripts, which function as the primer and template sequences, respectively. All components of the retron system are expressed from a single open reading frame as a single transcript including the msr-msd and encoding the retron RT protein (Lampson, et al., 2005, Retrons, msDNA, and the bacterial genome. Cytogenet Genome Res 110:491-499). The msr element ORF of a retron provides for the RNA portion of the msDNA molecule, while the msd element ORF provides for the DNA portion of the msDNA molecule. The primary transcript from the msr-msd region is thought to serve as both a template and a primer to produce the msDNA. Synthesis of msDNA is primed from an internal rG residue of the RNA transcript using its 2′-OH group. Modification of msd, or msr may also be made to permit insertion of a RNA template encoding a donor polynucleotide within the msd without altering the functioning of or the production of msDNA. The RNA template encoding a donor polynucleotide sequence may be any length but is preferably less than about 5 kb nucleotides, or also less than about 2 kb, or also less than 500 bases, provided that an msDNA product is produced.

### Diversity Generating Retroelements (DGRS)

In an embodiment, the one or more functional domains may be a diversity generating retroelement(s) (e.g., DGR described in US20100041033A1). In one embodiment, the DGR may insert a donor polynucleotide with its homing mechanism. For example, the DGR may be associated with a catalytically inactive IscB protein (e.g., a dead IscB), and integrate the single-strand DNA using a homing mechanism. In some examples, the DGR may be less mutagenic than a counterpart wild type DGR. In some examples, the DGR is not error-prone. In one embodiment, the DGR herein is not mutagenic. The non-mutagenic DGR may be a mutant of a wild type DGR. As used herein, the term “DGR” encompasses both diversity generating retroelement polynucleotides and proteins encoded by diversity generating retroelement polynucleotides. In some examples, DGR may be proteins encoded by diversity generating retroelement polynucleotides having reverse transcriptase activity. In some examples, DGR may be proteins encoded by diversity generating retroelement polynucleotides having reverse transcriptase activity and integrase activity. In some cases, the template or donor polynucleotide may be encoded by a diversity generating retroelement polynucleotide. In certain cases, the template may be a polynucleotide different from the diversity generating retroelement polynucleotide, e.g., provided as a separate construct or molecule.

In one embodiment, the DGR herein may also include a Group II intron (and any proteins and polynucleotides encoded), which are mobile ribozymes that self-splice from precursor RNAs to yield excised intron lariat RNAs, which then invade new genomic DNA sites by reverse splicing. Examples of Group II intron include those described in Lambowitz A M et al., Group II Introns: Mobile Ribozymes that Invade DNA, Cold Spring Harb Perspect Biol. 2011 August; 3 (8): a003616.

In one embodiment, the diversity-generating retroelements (DGRs) are genetic elements that can produce targeted, massive variations in the genomes that carry these elements. In one embodiment, the DGR systems rely on error-prone reverse transcriptases to produce mutagenized cDNA (containing A-to-N mutations) from a template region (TR), to replace a segment called a variable region (VR) that is similar to the TR region—this process is called mutagenic retrohoming (see, e.g., Sharifi and Ye, MyDGR: a server for identification and characterization of diversity-generating retroelements. Nucleic Acids Res. 2019 Jul. 2; 47 (W1): W289-W294). DGRs may include a unique family of retroelements that generate sequence diversity of DNA. They exist widely in bacteria, archaea, phage and plasmid, and benefit their hosts by introducing variations and accelerating the evolution of target proteins (see, e.g., Yan et al., Discovery and characterization of the evolution, variation and functions of diversity-generating retroelements using thousands of genomes and metagenomes. BMC Genomics. 2019; 20:595). The first DGR was discovered in a Bordetella phage, BPP-1. Bordetella causes the respiratory infection in humans and many other mammals, controlled by the BvgAS signal transduction system. The surface of Bordetella is highly variable owing to the dynamic gene expression in the infectious cycle. The invasion of BPP-1 to Bordetella relies on the phage tail fiber protein Mtd. With the process of mutagenic reverse transcription and cDNA integration, DGR may introduce multiple nucleotide substitutions to Mtd gene and generates different receptor-binding molecules, thus making BPP-1 the ability to invade Bordetellae with diverse cell surfaces.

The systems may be used to generate an ssDNA donor using a retron- or DGR RT, which is then integrated by homologous recombination upon target cleavage or nicking using a IscB nuclease. In one embodiment, the systems may comprise DGRs and/or Group-II intron reverse transcriptases. The homing mechanism of DGRs or Group-II introns may be used in modifying a target polynucleotide. The DGRs or Group-II introns reverse transcriptase may be guided to a target polynucleotide by tethering to a nuclease-dead IscB nuclease, TALE, or ZF protein. In another embodiment, a non-retron/DGR reverse transcriptase (e.g. a viral RT) may be used for generating cDNA off of a self-priming RNA. In one embodiment, a ssDNA may be generated by an RT, but integrate it using a dead IscB polypeptide or CRISPR-associated IscB polypeptide, creating an accessible R-loop instead of nicking/cleaving.

### Topoisomerases

The one or more functional domains may be one or more topoisomerase domains. In one embodiment, an engineered system for modifying a target polynucleotide comprising: a IsrB polypeptide; a topoisomerase domain; and a nucleic acid template comprising or encoding a donor polynucleotide to be inserted to a target sequence of the target polynucleotide. In some examples, two or more of: the IsrB polypeptide; topoisomerase domain; and nucleic acid template may form a complex. In some examples, two or more of: the IsrB; topoisomerase domain, may be comprised in a fusion protein.

Topoisomerases are a class of enzymes that modify the topological state of DNA via the breakage and rejoining of nucleic acid strands. In some cases, a topoisomerase may be a DNA topoisomerase, which is an enzyme that controls and alters the topologic states of DNA during transcription, and catalyzes the transient breaking and rejoining of a single strand of DNA which allows the strands to pass through one another, thus altering the topology of DNA.

In one embodiment, the topoisomerase domain is capable of ligating the donor polynucleotide with the target polynucleotide. The ligation may be achieved by sticky end or blunt end ligation. In an example, the donor polynucleotide may comprise an overhang comprising a sequence complementary to a region of the target polynucleotide. Examples of ligating the donor polynucleotide with the target polynucleotide include those of TOPO cloning, e.g., those described in “The Technology Behind TOPO Cloning,” at www.thermofisher.com/us/en/home/life-science/cloning/topo/topo-resources/the-technology-behind-topo-cloning.html.

In one embodiment, the topoisomerase domain may be associated with the donor polynucleotide. For example, the topoisomerase domain is covalently linked to the donor polynucleotide.

In one embodiment, a topoisomerase domain may be provided together with, e.g., associated (e.g., fused) with a IsrB polypeptide. Alternatively or additionally, the topoisomerase domain may be on a molecule different from the IsrB polypeptide. In some cases, the topoisomerase domain may be associated with a donor polynucleotide. For example, the topoisomerase domain may be pre-loaded covalently with a donor DNA molecule. Such design may allow for efficient ligation of only a specific cargo. The topoisomerase domain may ligate the donor polynucleotide (e.g., a DNA molecule) to a target site on a target polynucleotide (e.g., a free double-stranded DNA end). In one embodiment, the donor polynucleotide may have an overhang that comprises a sequence complementary to a region of the target polynucleotide. For example, the overhang may invade into the target polynucleotide at a cut site generated by the IsrB polypeptide.

Examples of topoisomerases include type I, including type IA and type IB topoisomerases, which cleave a single strand of a double-stranded nucleic acid molecule, and type II topoisomerases (e.g., gyrases), which cleave both strands of a double-stranded nucleic acid molecule.

Type IA and IB topoisomerases cleave one strand of a double-stranded nucleic acid molecule. In some examples, the cleavage of a double-stranded nucleic acid molecule by type IA topoisomerases generates a 5′ phosphate and a 3′ hydroxyl at the cleavage site, with the type IA topoisomerase covalently binding to the 5′ terminus of a cleaved strand. Cleavage of a double-stranded nucleic acid molecule by type IB topoisomerases may generate a 3′ phosphate and a 5′ hydroxyl at the cleavage site, with the type IB topoisomerase covalently binding to the 3′ terminus of a cleaved strand.

Examples of Type IA topoisomerases include E. coli topoisomerase I, E. coli topoisomerase III, eukaryotic topoisomerase II, archeal reverse gyrase, yeast topoisomerase III, Drosophila topoisomerase III, human topoisomerase III, Streptococcus pneumoniae topoisomerase III, and the like, including other type IA topoisomerases. A DNA-protein adduct is formed with the enzyme covalently binding to the 5′-thymidine residue, with cleavage occurring between the two thymidine residues.

Examples of Type IB topoisomerases include the nuclear type I topoisomerases present in all eukaryotic cells and those encoded by Vaccinia and other cellular poxviruses. The eukaryotic type IB topoisomerases are exemplified by those expressed in yeast, Drosophila and mammalian cells, including human cells. Viral type IB topoisomerases are exemplified by those produced by the vertebrate poxviruses (Vaccinia, Shope fibroma virus, ORF virus, fowlpox virus, and molluscum contagiosum virus), and the insect poxvirus (Amsacta moorei entomopoxvirus).

Examples of Type II topoisomerases include, bacterial gyrase, bacterial DNA topoisomerase IV, eukaryotic DNA topoisomerase II, and T-even phage encoded DNA topoisomerases. Type II topoisomerases may have both cleaving and ligating activities. Substrate double-stranded nucleic acid molecules of type II topoisomerase can be prepared such that the type II topoisomerase can form a covalent linkage to one strand at a cleavage site. For example, calf thymus type II topoisomerase can cleave a substrate ds nucleic acid molecule containing a 5′ recessed topoisomerase recognition site positioned three nucleotides from the 5′ end, resulting in dissociation of the three nucleic acid molecule 5′ to the cleavage site and covalent binding of the topoisomerase to the 5′ terminus of the ds nucleic acid molecule. Furthermore, upon contacting such a type II topoisomerase-charged ds nucleic acid molecule with a second nucleic acid molecule containing a 3′ hydroxyl group, the type II topoisomerase can ligate the sequences together, and then is released from the recombinant nucleic acid molecule.

In some examples, the topoisomerase is DNA topoisomerase I, e.g., a Vaccinia virus topoisomerase I. The topoisomerase may be pre-loaded with a donor polynucleotide. The Vaccinia virus topoisomerase may need a target comprising a 5′—OH group.

### Phosphatases

The systems herein may further comprise a phosphatase domain. A phosphatase is an enzyme capable of removing a phosphate group from a molecule e.g., a nucleic acid such as DNA. Examples of phosphatases include calf intestinal phosphatase, shrimp alkaline phosphatase, Antarctic phosphatase, and APEX alkaline phosphatase.

In some examples, the 5′—OH group of in the target polynucleotide may be generated by a phosphatase. A topoisomerase compatible with a 5′ phosphate target may be used to generate stable loaded intermediates. In some cases, a IscB polypeptide or CRISPR-associated IscB polypeptide nuclease that leaves a 5′ OH after cleaving the target polynucleotide may be used. In some cases, the phosphatase domain may be associated with (e.g., fused to) the IscB protein. The phosphatase domain may be capable of generating a —OH group at a 5′ end of the target polynucleotide. The phosphatase may be delivered separated from other components in the system, e.g., as a separate protein, on a separate vector from other components.

### Polymerases

The systems herein may further comprise a polymerase domain. A polymerase refers to an enzyme that synthesizes chains of nucleic acids. The polymerase may be a DNA polymerase or an RNA polymerase.

In one embodiment, the systems comprise an engineered system for modifying a target polynucleotide comprising: a IsrB polypeptide; a DNA polymerase domain; and a DNA template comprising a donor polynucleotide to be inserted to a target sequence of the target polynucleotide. In some examples, two or more of: the IscB protein; DNA polymerase domain; and DNA template may form a complex. In some examples, two or more of: the IsrB protein; DNA polymerase domain; are comprised in a fusion protein. For example, the IsrB polypeptide and DNA polymerase domain may be comprised in a fusion protein.

In one embodiment, the systems may comprise a IsrB polypeptide nuclease (or variant thereof such as a dIsrB polypeptide and a DNA polymerase (e.g. phi29, T4, T7 DNA polymerase). The systems may further comprise a single-stranded DNA or double-stranded DNA template. The DNA template may comprise i) a first sequence homologous to a target site of the IscB protein on the target polynucleotide, and/or ii) a second sequence homologous to another region of the target polynucleotide. In one embodiment, the template may be a synthetic single-stranded or PCR-generated DNA molecule, (optionally end-protected by modified nucleotides), or a viral genome (e.g. AAV). In another embodiment, the template is generated using a reverse transcriptase. When the system is delivered into a cell, an endogenous DNA polymerase in the cell may be used. Alternatively or additionally, an exogenous DNA polymerase may be expressed in the cell.

The DNA template may be end-protected by one or more modified nucleotides, or comprises a portion of a viral genome. In some embodiment, the DNA template comprises LNA or other modifications (e.g., at the 3′ end). The presence of LNA and/or the modifications may lead to more efficient annealing with the 3′ flap generated by IscB polypeptide or CRISPR-associated IscB polypeptide cleavage.

Examples of DNA polymerase include Taq, Tne (exo-), Tma (exo-), Pfu (exo-), Pwo (exo-), Thermoanaerobacter thermohydrosulfuricus DNA polymerase, Thermococcus litoralis DNA polymerase I, E. coli DNA polymerase I, Taq DNA polymerase I, Tth DNA polymerase I, Bacillus stearothermophilus (Bst) DNA polymerase I, E. coli DNA polymerase III, bacteriophage T5 DNA polymerase, bacteriophage M2 DNA polymerase, bacteriophage T4 DNA polymerase, bacteriophage T7 DNA polymerase, bacteriophage phi29 DNA polymerase, bacteriophage PRD1 DNA polymerase, bacteriophage phi15 DNA polymerase, bacteriophage phi21DNA polymerase, bacteriophage PZE DNA polymerase, bacteriophage PZA DNA polymerase, bacteriophage Nf DNA polymerase, bacteriophage M2Y DNA polymerase, bacteriophage B103 DNA polymerase, bacteriophage SF5 DNA polymerase, bacteriophage GA-1 DNA polymerase, bacteriophage Cp-5 DNA polymerase, bacteriophage Cp-7 DNA polymerase, bacteriophage PR4 DNA polymerase, bacteriophage PR5 DNA polymerase, bacteriophage PR722 DNA polymerase and bacteriophage L17 DNA polymerase.

### Ligases

In general, the systems comprise a IsrB polypeptide and a ligase associated with the IscB protein. The IsrB polypeptide may be recruited to the target sequence by an ωRNA, and generate a break on the target sequence. The ωRNA may further comprise a template sequence with desired mutations or other sequence elements. The template sequence may be ligated to the target sequence to introduce the mutations or other sequence elements to the nucleic acid molecule. The IsrB polypeptide may be a nickase that generates a single-strand break on nucleic acid molecule, and the ligase may be a single-strand DNA ligase. In one embodiment, the systems comprise a pair of IsrB polypeptide-ligases complexed with two distinct ωRNA sequences. Each IsrB polypeptide ligase complex can target one strand of a double-stranded polynucleotide, and work together to effectively modify the sequence of the double-stranded polynucleotides.

In some examples, the IsrB polypeptide is associated with a ligase or functional fragment thereof. The ligase may ligate a single-strand break (a nick) generated by the IsrB polypeptide. In certain examples, the IsrB polypeptide is associated with a reverse transcriptase or functional fragment thereof.

The present invention further provides systems and methods of modifying a nucleic acid sequence using a pair of distinct IsrB polypeptide-ligase-@ RNA complexes, said systems and methods comprising: (a) an engineered IsrB polypeptide connected to or complexed with a ligase; (b) two distinct ωRNA sequences complexed with such IsrB polypeptide-ligase protein complex to form a first and a second distinct IsrB-ligase ωRNA complexes; (c) the first IsrB-ligase ωRNA complex binding to one strand of a target double-stranded polynucleotide sequence, and the second IsrB polypeptide-ligase-ωRNA complex binding to another strand of the target double-stranded polynucleotide sequence; (d) upon binding of the said complexes to the locus of interest the effector protein induces the modification of the sequences associated with or at the target locus of interest, whereby the two IsrB polypeptide-ligase-ωRNA complexes work together on different strands of the double-stranded target sequence and modify the sequence.

One of the advantages of using such a “pair” of IsrB polypeptide-ligase-ωRNA complexes includes high efficiency in modifying the sequence associated with or at the locus of interest of target double-stranded polynucleotides.

In one embodiment, the IsrB polypeptide can be a nickase. In a preferred embodiment, a ligase is linked to the IsrB polypeptide. The ligase can ligate the donor sequence to the target sequence. The ligase can be a single-strand DNA ligase or a double-strand DNA ligase. The ligase can be fused to the carboxyl-terminus of a IsrB polypeptide, or to the amino-terminus of a IsrB polypeptide.

As used herein the term “ligase” refers to an enzyme, which catalyzes the joining of breaks (e.g., double-stranded breaks or single-stranded breaks (“nicks”) between adjacent bases of nucleic acids. For example, a ligase may be an enzyme capable of forming intra- or inter-molecular covalent bonds between a 5′ phosphate group and a 3′ hydroxyl group. The term “ligate” refers to the reaction of covalently joining adjacent oligonucleotides through formation of an internucleotide linkage.

DNA ligases fall into two general categories: ATP-dependent DNA ligases (EC 6.5.1.1), and NAD (+) dependent DNA ligases (EC 6.5.1.2). NAD (+) dependent DNA ligases are found only in bacteria (and some viruses) while ATP-dependent DNA ligases are ubiquitous. The ATP-dependent DNA ligases can be divided into four classes: DNA ligase I, II, III, and IV. DNA ligase I links Okazaki fragments to form a continuous strand of DNA; DNA ligase II is an alternatively spliced form of DNA ligase III, found only in non-dividing cells; DNA ligase III is involved in base excision repair; and DNA ligase IV is involved in the repair of DNA double-strand breaks by non-homologous end joining (NHEJ). Amongst all ligases, there are two types of prokaryotic and one type of eukaryotic ligases that are particularly well suited for facilitating the blunt-ended, double-stranded DNA ligation: Prokaryotic DNA ligases (T3 and T4) and Eukaryotic DNA ligase (Ligase 1).

In some cases, the ligase is specific for double-stranded nucleic acids (e.g., dsDNA, dsRNA, RNA/DNA duplex). An example of a ligase specific for double-stranded DNA and DNA/RNA hybrids is T4 DNA ligase. In some cases, the ligase is specific for single-stranded nucleic acids (e.g., ssDNA, ssRNA). An example of such ligase is CircLigase II. In some cases, the ligase is specific for RNA/DNA duplexes. In some cases, the ligase is able to work on single-stranded, double-stranded, and/or RNA/DNA nucleic acids in any combination.

In some cases, the ligase may be a pan-ligase, which is a single ligase with the ability to ligate both DNA and RNA targets. The ligase may be specific for a target (e.g., DNA-specific or RNA-specific). In some cases, the ligase may be a dual ligase system that include DNA-specific, RNA-specific, and/or pan-ligases, in any combination.

Examples of ligases that can be used with the disclosure include T4 DNA Ligase, T3 DNA Ligase, T7 DNA Ligase, E. coli DNA Ligase, HiFi Taq DNA Ligase, 9° N™ DNA Ligase, Taq DNA Ligase, SplintR® Ligase (also known as. PBCV-1 DNA Ligase or Chlorella virus DNA Ligase), Thermostable 5′ AppDNA/RNA Ligase, T4 RNA Ligase, T4 RNA Ligase 2, T4 RNA Ligase 2 Truncated, T4 RNA Ligase 2 Truncated K227Q, T4 RNA Ligase 2, Truncated KQ, RtcB Ligase (joins single stranded RNA with a 3″-phosphate or 2′,3′-cyclic phosphate to another RNA), CircLigase II, CircLigase ssDNA Ligase, CircLigase RNA Ligase, or Ampligase® Thermostable DNA Ligas, NAD-dependent ligases including Taq DNA ligase, Thermus filiformis DNA ligase, Escherichia coliDNA ligase, Tth DNA ligase, Thermus scotoductus DNA ligase (I and II), thermostable ligase, Ampligase thermostable DNA ligase, VanC-type ligase, 9° N DNA Ligase, Tsp DNA ligase, and novel ligases discovered by bioprospecting; ATP-dependent ligases including T4 RNA ligase, T4 DNA ligase, T3 DNA ligase, T7 DNA ligase, Pfu DNA ligase, DNA ligase I, DNA ligase III, DNA ligase IV, and novel ligases discovered by bioprospecting, and wild-type, mutant isoforms, and genetically engineered variants thereof. In a particular example, the ligase is a

In one embodiment, the examples of the ligases include those used in sequencing by synthesis or sequencing by ligation reactions.

### Helitrons

The systems and compositions herein may comprise a IsrB polypeptide nuclease, one or more @ RNAs, and one or more components of a helitron. The systems and compositions may be used to insert a donor polynucleotide to a target polynucleotide. The systems and compositions may further comprise a donor polynucleotide.

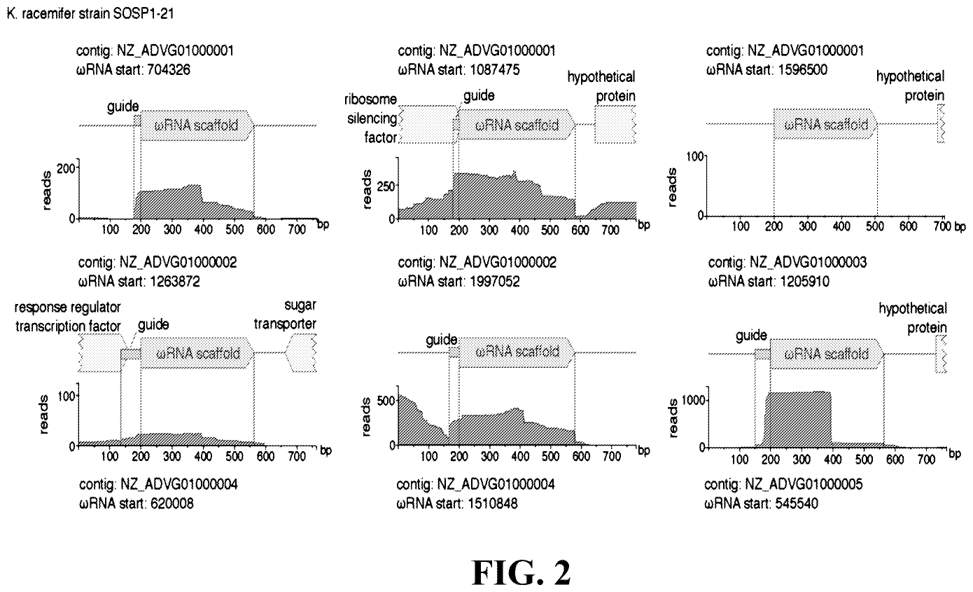

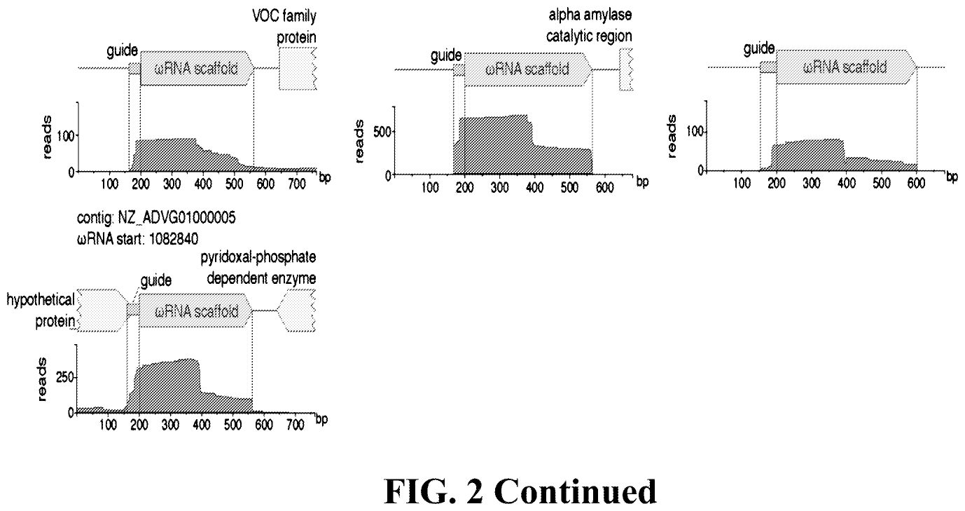

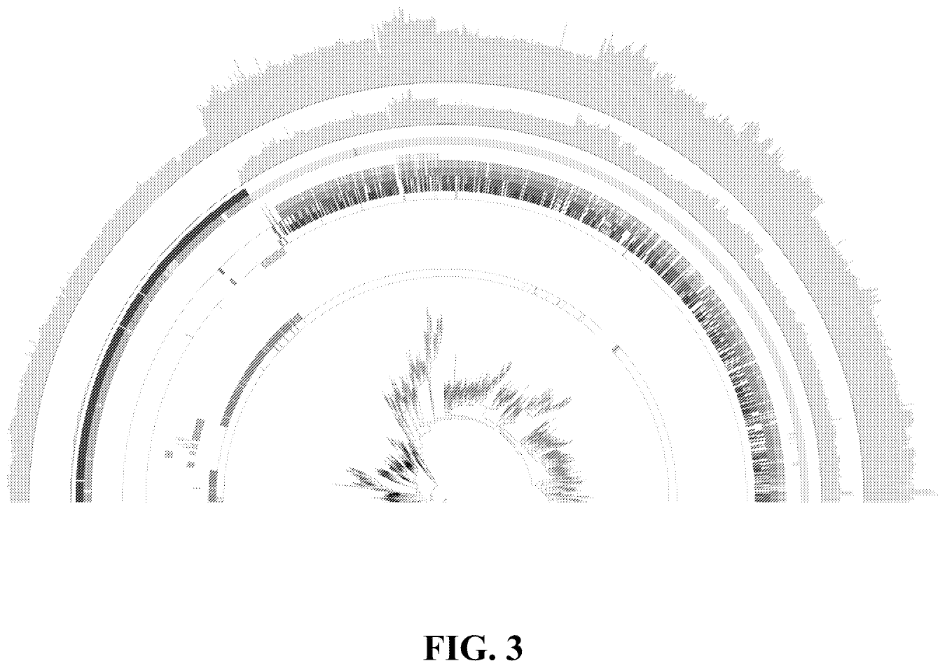

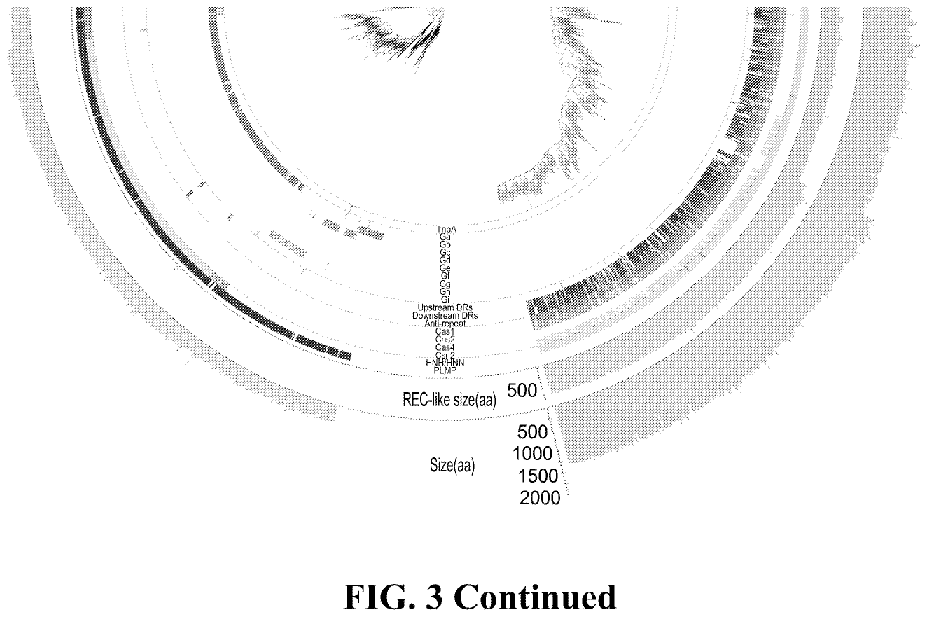

The term “helitron”, as used herein, refers to a polynucleotide (or nucleic acid segment), recognized as a transposon that captures and mobilizes gene fragments in eukaryotes. The term “helitron” as used herein refers to transposase that comprises an endonuclease domain and a C-terminal helicase domain. Helitrons are rolling-circle RNA transposons. In one embodiment, the helitron encodes a 1400 to about 2000 amino acid, or about 1800 amino acid multidomain transposase. In embodiments, the helitron comprises a hairpin near the 3′end to function as a transposition terminator. In embodiments, the transposon comprises a RepHel motif comprising a replication initiator (Rep) and a DNA helicase (hel) domain. See, Thomas J. & Pritham E. J. Helitrons, the eukaryotic rolling-circle transposable elements. Microbiol. Spectr. 3, 893-926 (2015). In embodiments, the helitron comprises a Rep nuclease domain and C-terminal helicase domain and inserts between an AT dinucleotide in single strand DNA. In an aspect, the C-terminal helicase unwinds the DNA in a 5′ to 3′ direction. The HUH nuclease domain may comprise one or two active site tyrosine residues, in embodiments, is a 2 Tyrosine (Y2) HUH endonuclease domain. Helitrons can encompass helentron, proto-helentron and helitron2 type proteins, structures of which can be as described in Thomas et al., 2015 at FIGS. 1 and 3, incorporated specifically by reference. Particular organsisms in which the helitron or helentrons have been found can include those in Table 1 of Thomas J. & Pritham E. J. Helitrons, the eukaryotic rolling-circle transposable elements. Microbiol. Spectr. 3, 893-926 (2015), incorporated herein by reference. Similarly, helitrons can be identified based at least in part on the Rep motif, and conserved residues in the helitrons, and according to the alignment sequence of FIG. 2 of Thomas J. & Pritham E. J. Helitrons, the eukaryotic rolling-circle transposable elements. Microbiol. Spectr. 3, 893-926 (2015), specifically incorporated herein by reference.

The expression “helitron reaction” used herein refers to a reaction wherein a transposase inserts a donor polynucleotide sequence in or adjacent to an insertion site on a target polynucleotide. The insertion site may contain a sequence or secondary structure recognized by the helitron and/or an insertion motif sequence in the target polynucleotide into which the donor polynucleotide sequence may be inserted.

As described in Grabundzija 2018, the helitron terminal sequences contains a distinct ˜150 base pairs (bp) long sequence with an absolutely conserved dinucleotide at the end of left terminal sequence (LTS), and a tetranucleotide at the end of right terminal sequence (RTS) which is preceded by a palindromic sequence that can form a hairpin structure. Grabundzija et al., Nat. Commun. 2018; 9:1278; doi: 10.1035/s41467-018-03688-w.

The helitron end sequences may be responsible for identifying the donor polynucleotide for transposition. The helitron end sequences may be the DNA sequences used to perform a transposition reaction, the end sequences may be referred to herein as right terminal sequences and left terminal sequence. The donor polynucleotide can be configured to comprise a first and second helitron recognition sequence that are at least 80%, 85%, 90%, 95% 96%, 97%, 98%, 99% or 100% complementary to a left terminal sequence and/or a right terminal sequence of a polynucleotide encoding the helitron polypeptide.

In an aspect, the palindromic sequence may be located upstream of the right terminal sequence, for example, about 5, 10, 15, 20, 25, 30, 35 nucleotides upstream of the right terminal sequence end, or about 10 to 15 nucleotides upstream of the right terminal sequence end, about 10 to 12 nucleotides or about 11 nucleotides upstream of the right terminal sequence end. Ivana Grabundzija, Nat Commun. 2016; 7:10716, doi: 10.1038/ncomms10716, incorporated herein by reference.

Exemplary helitrons can be identified using software, for example (EAHelitron) that has been used to identify Helitrons in a wide range of plant genomes. See, Hu, K., Xu, K., Wen, J. et al. Helitron distribution in Brassicaceae and whole Genome Helitron density as a character for distinguishing plant species. BMC Bioinformatics 20, 354 (2019). doi: 10.1186/s12859-019-2945-8, incorporated herein by reference.

The helitron may be derived from a eukaryote. In an aspect, the helitron is derived from a mammalian genome, in an aspect, vespertilionid bats, e.g. Helibat. In embodiments, the helitron is derived from derived from a Helibat1 transposon. In embodiments, the helitron is Helraiser, the full DNA sequence of the consensus transposon, including left terminal and right terminal sequences as well as hairpin identified is provided in Grabundzija, 2016 at Supplementary FIG. 1, specifically incorporated herein by reference. In an aspect, the helitron is flanked by left and right terminal sequences of the transposon. In an aspect, the left terminal sequence and right terminal sequence terminates with the conserved 5′-TC/CTAG-3′ motif. In an embodiment, the helitron may comprise a palindromic sequence that is about 10 to about 35, or about 5-25 bp or about 19-bp-long palindromic sequence with the potential to form a hairpin structure.

Elements of these systems may be engineered to work within the context of the invention. For example, a helitron polypeptide may be fused to a polypeptide capable of generating an R-loop. Fusion may be by any appropriate linker, in an exemplary embodiment, XTEN16. The binding elements that allow a helitron polypeptide to bind, for example, the use of sequences complementary to the right terminal sequence and the left terminal sequence of the helitron may be engineered into a donor construct to facilitate entry of a donor polynucleotide sequence into a target polynucleotide.

In certain example embodiments, the Isc polypeptide, via formation of complex with a hRNA sequence, directs the helitron polypeptide to a target sequence in a target polynucleotide, where the helitron facilitates integration of a donor polynucleotide sequence into the target polynucleotide.

The helitron polypeptides may also comprise one or more truncations or excisions to remove domains or regions of wild-type protein to arrive at a minimal polypeptide, alter functionality according to the system in which the helitron is used, or mutated to enhance or diminish particular activities associated with the helitron, i.e. nuclease activity or helicase activity.

### IsrB Recombinase

The systems and compositions herein may comprise a IsrB polypeptide system, and one or more components of a recombinase. In an aspect, the IsrB polypeptide is naturally catalytically inactive and utilized with one or more nucleic acid components to provide site-specific targetings, and the one or more components of the recombinase to introduce a modification. In an aspect, the IsrB polypeptide polypeptide may be catalytically inactivated via mutation of one or more residues of a catalytic domain or via truncation, and utilized with one or more RNA components to provide site-specific targeting, and the one or more components of the recombinase introduce a modification. In an aspect, the IsrB polypeptide may be catalytically inactivated, and utilized with one or more RNA components to provide site-specific targeting, with the one or more components of the recombinase introduce a modification. In preferred embodiments, the recombinatise mediates unidirectional site-specific recombination. In one embodiment, the recombinase is a serine recombinase (SR), encoded, for example, by IS607 family, Tn4451, and bacteriophage phiC31. See, generally, Smith M C, Thorpe H M: Diversity in the serine recombinases. Mol Microbiol. 2002, 44:299-307. 10.1046/j. 1365-2958.2002.02891.x; Li et al., (2018) J. Mol. Biol. 430:21, 4401-4418. In an embodiment, the recombinase is a tyrosine recombinase (YR) encoded by IS91, Helitron, IS200/IS605, Crypton or DIRS-retrotransposon families. See, generally, Goodwin T J, Butler M I, Poulter R T: Cryptons: a group of tyrosine-recombinase-encoding DNA transposons from pathogenic fungi. Microbiology. 2003, 149:3099-3109. Doi: 10.1099/mic.0.26529-0; Cappello J, Handelsman K, Lodish H F: Sequence of Dictyostelium DIRS-1: an apparent retrotransposon with inverted terminal repeats and an internal circle junction sequence. Cell. 1985, 43:105-115. 10.1016/0092-8674 (85) 90016-9. In an aspect, the recombinase provides site-specific integration of a template that can be provided with the composition, e.g. a donor oligonucleotide. Without being bound by theory, the recombinase allows for integration independent of payload size and can coordinate strand exchange and re-ligation across multiple cell types, allowing integration of long stretches of polynucleotides. In an exemplary embodiment, the serine recombinase is PhiC31 and the target is DNA. In an aspect, the phiC31 allows for integration of a target site comprising an attP or pseudoattP recognition site. See, e.g. systembio.com/wp-content/uploads/phiC31_productsheet-1.pdf. In an embodiment utilizing phiC231, a donor oligonucleotide would be provided with an attB at sequence that facilitates attachment at the attP site of the target genome. Similar approaches of designing donor oligonucleotides with sequences complementary to attachment sites for a recombinase can be designed for use with the present invention. See, e.g. Li et al., (2018) J. Mol. Biol. 430:21, 4401-4418.

In one embodiment, a naturally inactive IsrB is provided with an IS630 transposon. IS630 transposons comprise the DDE motif, which is an RNase H-like fold that draws three catalytically active residues DDE signature and average about 1100 bp in length. See, Gao, B., Wang, Y., Diaby, M. et al. Evolution of pogo, a separate superfamily of IS630-Tcl-mariner transposons, revealing recurrent domestication events in vertebrates. Mobile DNA 11, 25 (2020) doi: 10.1186/s13100-020-00220-0. In an aspect, the IS630 transposon provides high target specificity inserting into a TA dinucleotide. In an aspect, the insertion is at a 5′-NTAN-3′.

The systems and compositions herein may comprise a IsrB polypeptide, and one or more components of an integrase. In an aspect, the IsrB polypeptide is naturally catalytically inactive or is a nickase and is utilized with one or more nucleic acid components to provide site-specific targeting, and the one or more components of the integrase to introduce a modification. In an aspect, the IsrB polypeptide may be catalytically inactivated via mutation of one or more residues of a catalytic domain (e.g. RuvC domain) or via truncation, and utilized with one or more RNA components to provide site-specific targeting, and the one or more components of the recombinase introduce a modification. In an aspect, the IscB polypeptide is a nickase, and utilized with one or more RNA components to provide site-specific targeting, with the one or more components of the integrase introduce a modification. In an embodiment, the IsrB polypeptide is naturally catalytically inactive, for example, SEQ ID NOs. 1445-1523. In one embodiment, a naturally inactive IsrB is provided with an integrase, and optionally a reverse transcriptase.

In preferred embodiments, the integrase mediates gene integration at diverse loci by directing insertion with an IsrB nickase fused to both a reverse transcriptase and an integrase. In one embodiment, the integrase is a serine integrase, encoded, for example, BxbINT. See, generally, Ioannidi et al., “Drag-and-drop genome insertion without DNA cleavage with CRISPR-directed integrases”; doi: 10.1101/2021.11.01.466786m incorporated herein by reference in its entirety. In Ioannidi, Gootenberg, Abudayyeh, and colleagues show integration using a CRISPR-Cas9 nickase fused to a reverse transcriptase and serine integrase termed Programmable Addition via Site-specific Targeting Elements (PASTE) with delivery via a single dose of plasmids with functionality in non-dividing and primary cells, utilizing a guide RNA comprising an AttB landing site, termed attachment site-containing guide RNA were used to insert sequences, including diverse cargo sequences that can be inserted across different loci, varying in size up to about 36 kb. Additional uses of the PASTE system included gene tagging, gene replacement, gene delivery, and protein production and secretion, approaches that are contemplated for use with the IscB nickase and integrase approach. In an aspect, the omega RNA may comprise an AttB landing site. In an aspect, the recombinase provides site-specific integration of a template that can be provided with the composition, e.g. a donor oligonucleotide.

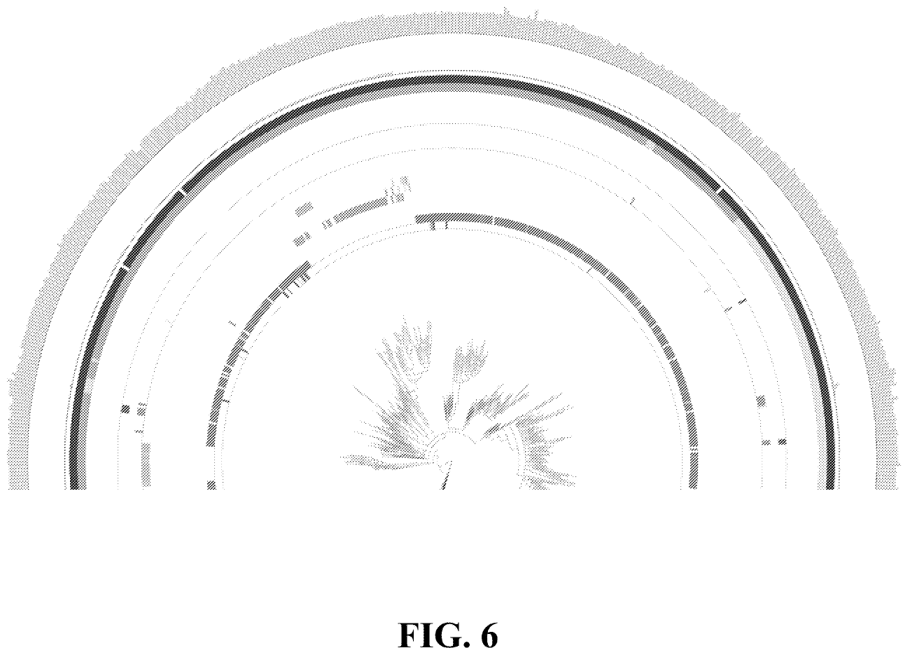

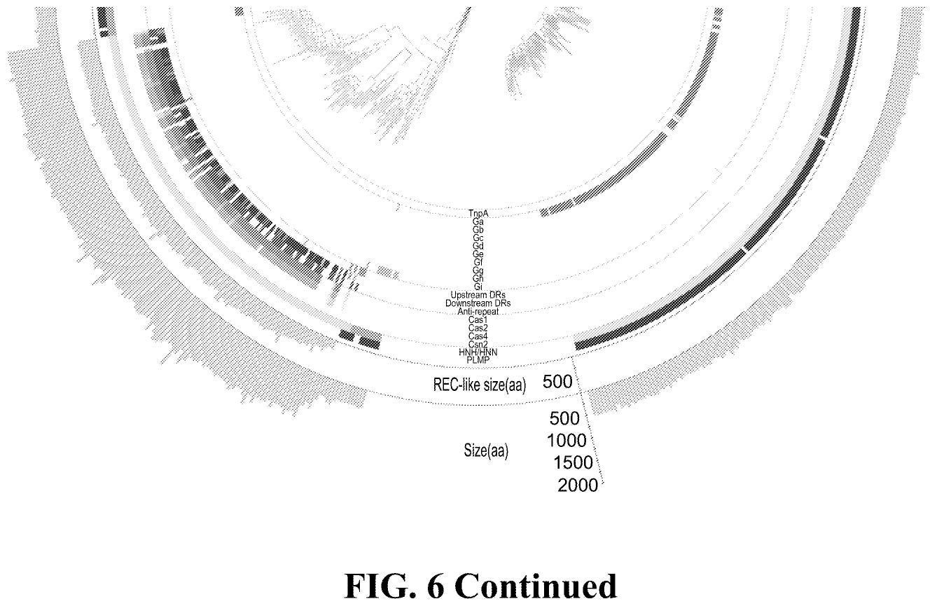

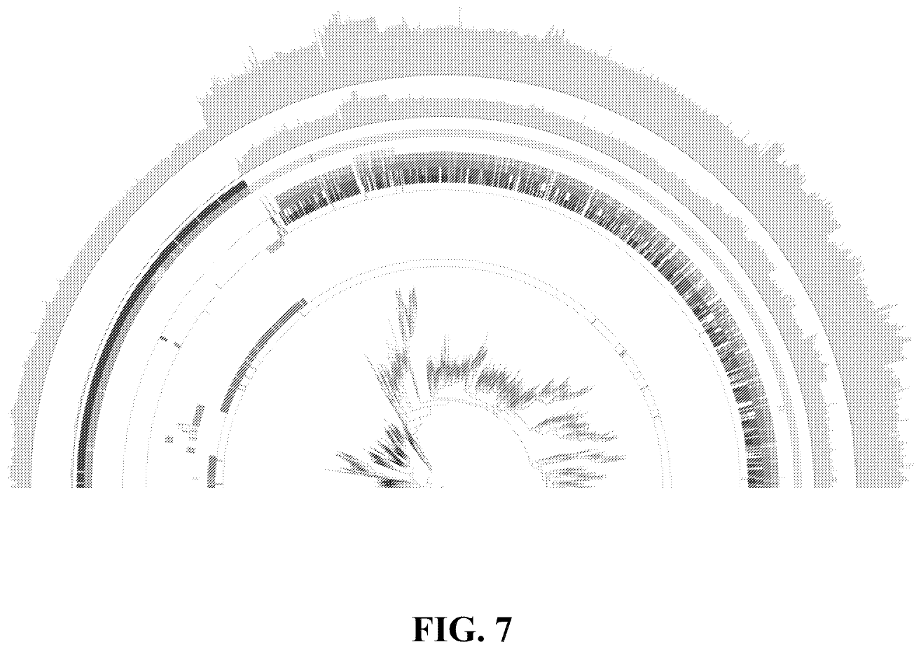

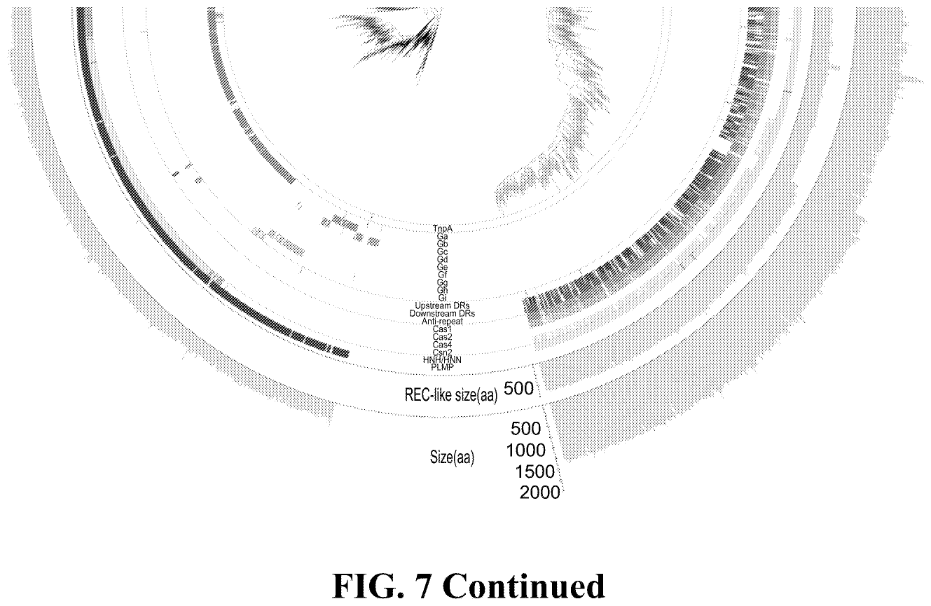

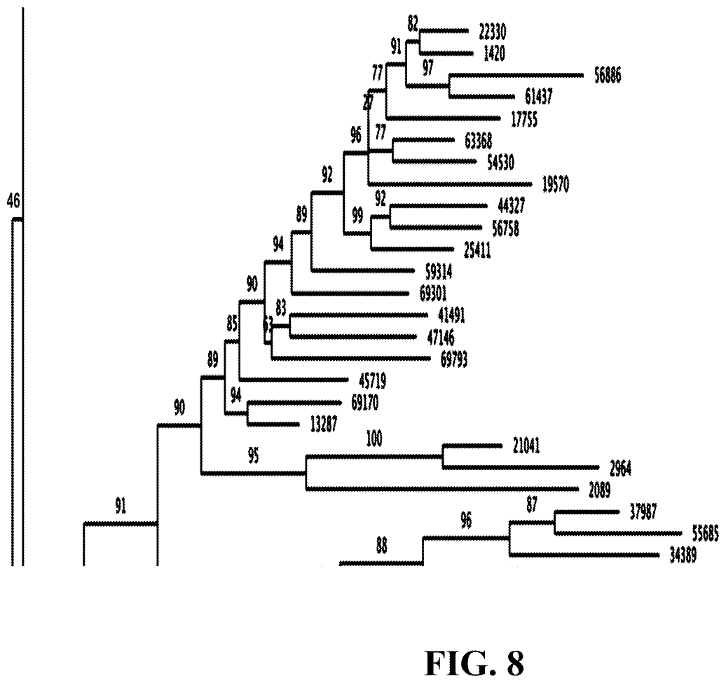

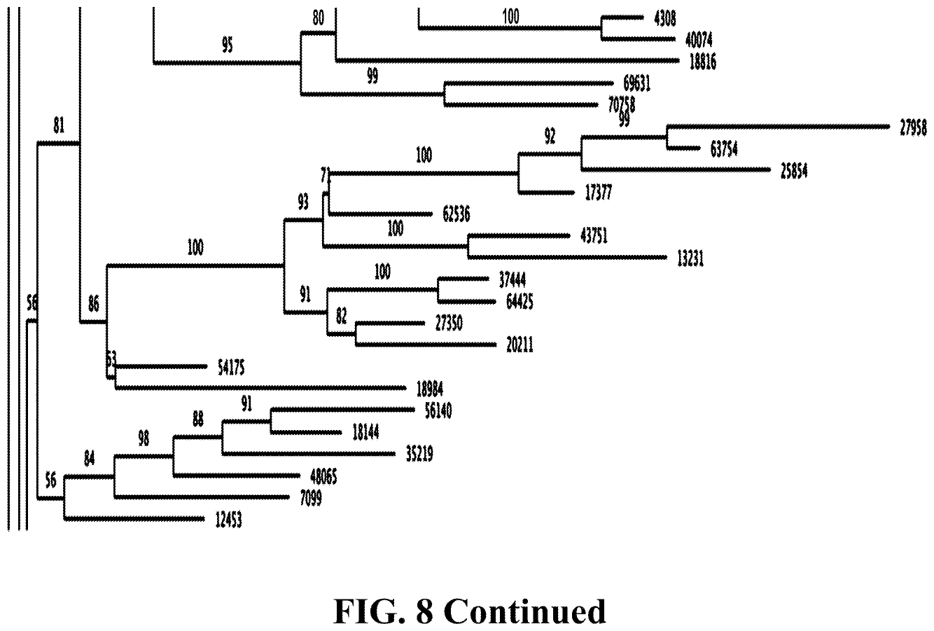

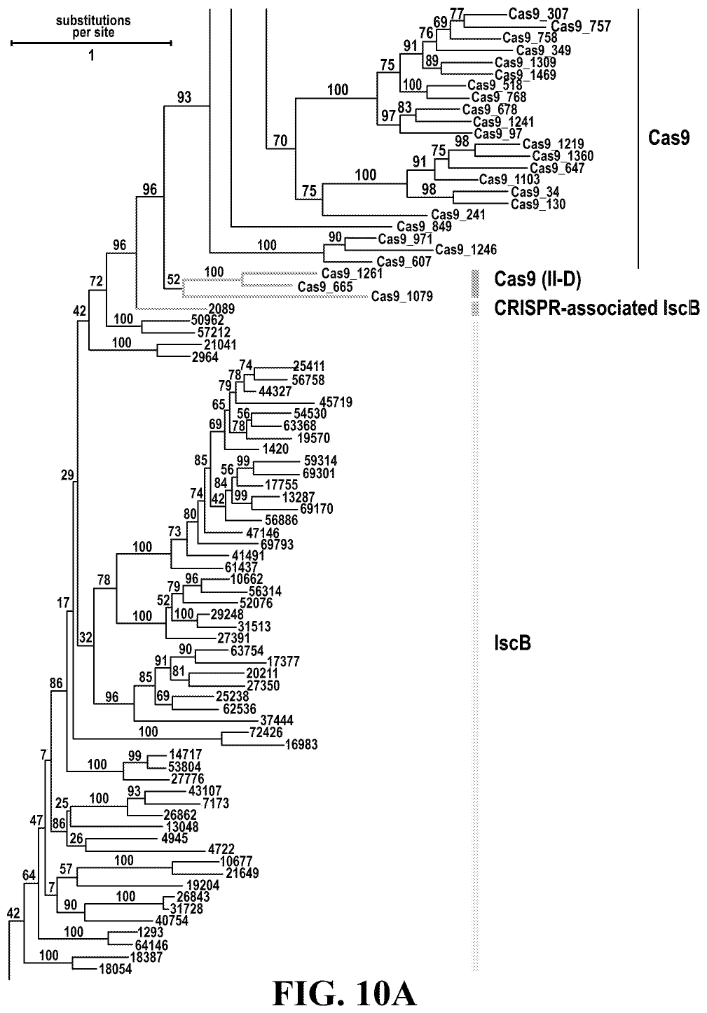

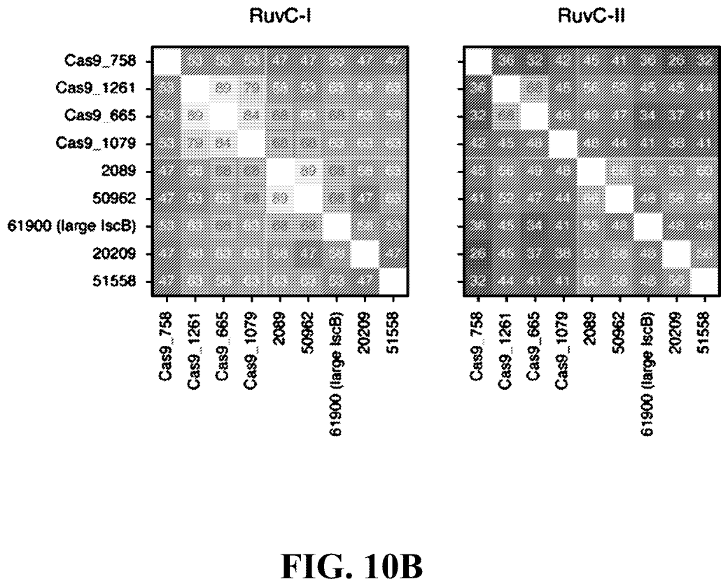

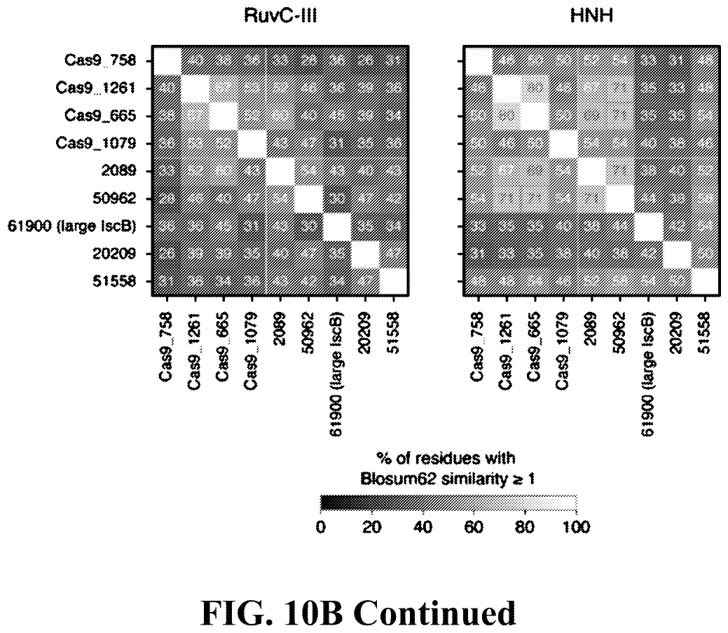

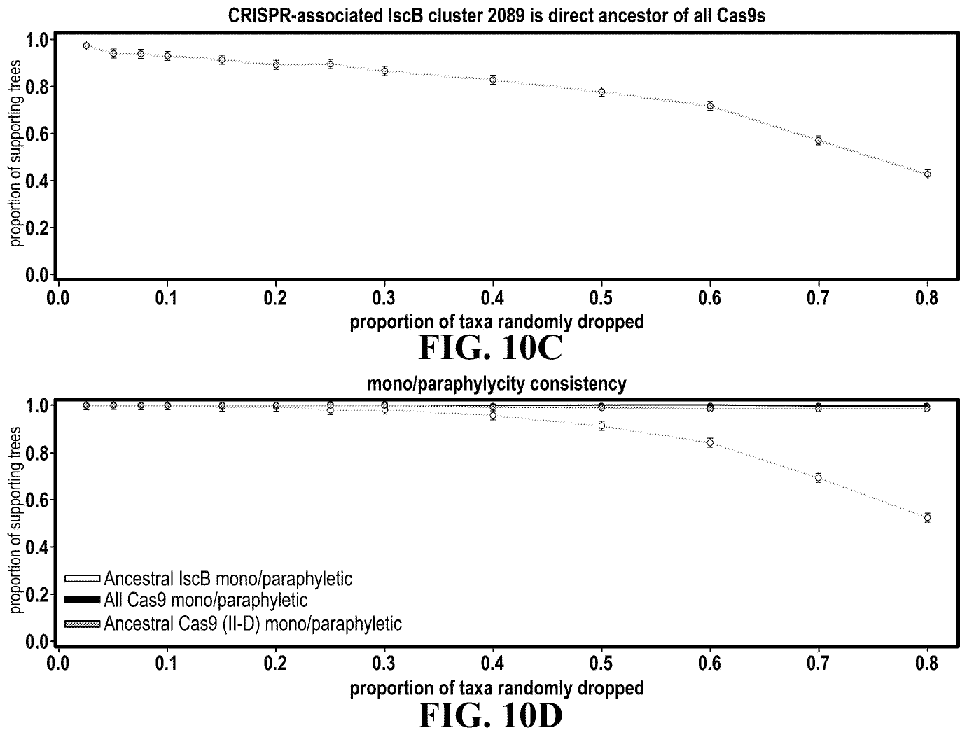

Additional large serine integrases can be used with the IsrB polypeptide, e.g. IsrB nickase, for example, as identified and described in Durrant et al., Large-scale discovery of recombinases for integrating DNA into the human genome, doi: 10.1101/2021.11.05.467528, incorporated herein by reference. Other integrases include BceINT, SscINT, SacINT. See, Ioannidi, 2021 at and FIG. 6d, and FIG. 10a.

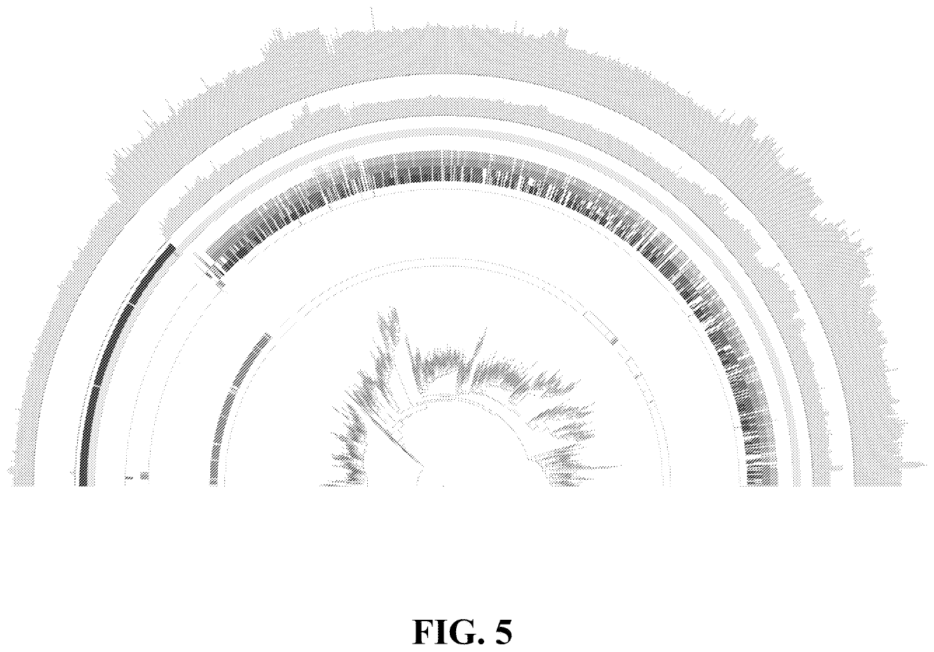

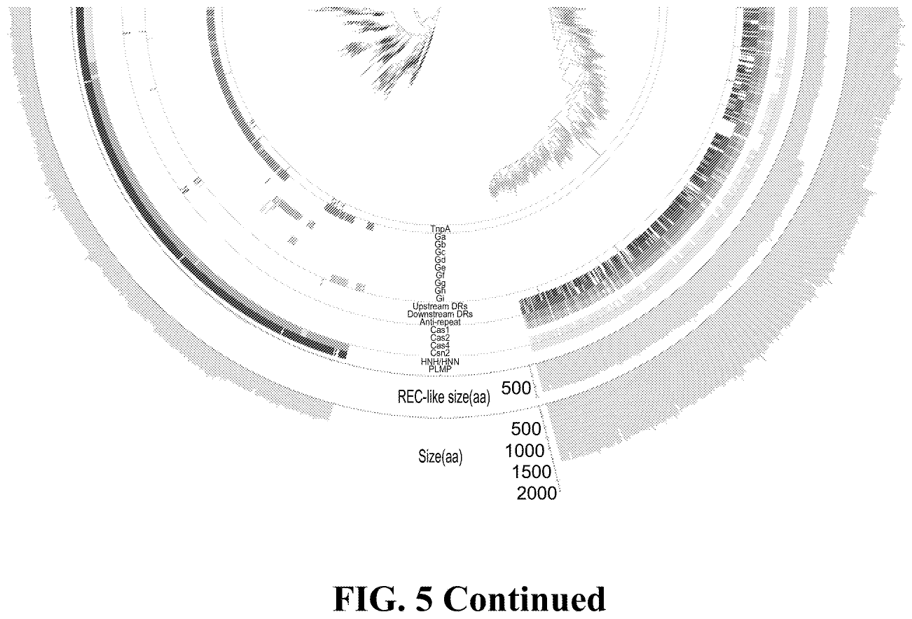

Without being bound by theory, the recombinase allows for integration independent of payload size and can coordinate strand exchange and re-ligation across multiple cell types, allowing integration of long stretches of polynucleotides. In an exemplary embodiment, the integrase is BxbINT and the target is DNA. In an aspect, the BxbINT allows for integration of a target site comprising an attP or pseudoattP recognition site. In an embodiment utilizing BxbINT, a donor oligonucleotide would be provided with an attB at sequence that facilitates attachment at the attP site of the target genome. Similar approaches of designing donor oligonucleotides with sequences complementary to attachment sites for an integrase can be designed for use with the present invention, for example a circular double-strand DNA template containing the AttP attachment site, or delivery of large cargo via an adenovirus or other viral vector, as described elsewhere herein. See, e.g. Ioannidi et al., 2021 at FIGS. 1a, 1b and 5b.

### Vectors Encoding IsrB Systems

The systems herein may comprise one or more polynucleotides. The polynucleotide(s) may comprise coding sequences of components of the systems herein, e.g., IsrB polypeptide nuclease, ωRNA(s), functional domain(s), donor polynucleotide(s), and/or other components in the systems. The present disclosure further provides vectors or vector systems comprising one or more polynucleotides herein. The vectors or vector systems include those described in the delivery sections herein.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. As used herein the term “wild type” is a term of the art understood by skilled persons and means the typical form of an organism, strain, gene or characteristic as it occurs in nature as distinguished from mutant or variant forms. A “wild type” can be a base line. As used herein the term “variant” should be taken to mean the exhibition of qualities that have a pattern that deviates from what occurs in nature. The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature. “Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y. Where reference is made to a polynucleotide sequence, then complementary or partially complementary sequences are also envisaged. These are preferably capable of hybridizing to the reference sequence under highly stringent conditions. “Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence. As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. For the purpose of this invention, it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. As used herein, “expression of a genomic locus” or “gene expression” is the process by which information from a gene is used in the synthesis of a functional gene product. The products of gene expression are often proteins, but in non-protein coding genes such as rRNA genes or tRNA genes, the product is functional RNA. The process of gene expression is used by all known life-eukaryotes (including multicellular organisms), prokaryotes (bacteria and archaea) and viruses to generate functional products to survive. As used herein “expression” of a gene or nucleic acid encompasses not only cellular gene expression, but also the transcription and translation of nucleic acid(s) in cloning systems and in any other context. As used herein, “expression” also refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. The terms “polypeptide”, “peptide” and “protein” are used interchangeably herein to refer to polymers of amino acids of any length. The polymer may be linear or branched, it may comprise modified amino acids, and it may be interrupted by non amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. As used herein, the term “domain” or “protein domain” refers to a part of a protein sequence that may exist and function independently of the rest of the protein chain. As described in aspects of the invention, sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences.

In an embodiment, the polynucleotide sequence is recombinant DNA. In further embodiments, the polynucleotide sequence further comprises additional sequences as described elsewhere herein. In an embodiment, the nucleic acid sequence is synthesized in vitro.

The present disclosure provides polynucleotide molecules that encode one or more components of the system or IsrB polypeptide nuclease as referred to in any embodiment herein. In an embodiment, the polynucleotide molecules may comprise further regulatory sequences. By means of guidance and not limitation, the polynucleotide sequence can be part of an expression plasmid, a minicircle, a lentiviral vector, a retroviral vector, an adenoviral or adeno-associated viral vector, a piggyback vector, or a tol2 vector. In an embodiment, the polynucleotide sequence may be a bicistronic expression construct. In further embodiments, the isolated polynucleotide sequence may be incorporated in a cellular genome. In yet further embodiments, the isolated polynucleotide sequence may be part of a cellular genome. In further embodiments, the isolated polynucleotide sequence may be comprised in an artificial chromosome. In an embodiment, the 5′ and/or 3′ end of the isolated polynucleotide sequence may be modified to improve the stability of the sequence of actively avoid degradation. In an embodiment, the isolated polynucleotide sequence may be comprised in a bacteriophage. In other embodiments, the isolated polynucleotide sequence may be contained in agrobacterium species. In an embodiment, the isolated polynucleotide sequence is lyophilized.

Aspects of the invention relate to polynucleotide molecules that encode one or more components of one or more systems as described in any of the embodiments herein, wherein at least one or more regions of the polynucleotide molecule may be codon optimized for expression in eukaryotic cells. In an embodiment, the polynucleotide molecules that encode one or more components of one or more systems as described in any of the embodiments herein are optimized for expression in a mammalian cell or a plant cell.

An example of a codon optimized sequence is in this instance a sequence optimized for expression in a eukaryote, e.g., humans (i.e. being optimized for expression in humans), or for another eukaryote, animal or mammal as herein discussed. In one embodiment, an enzyme coding sequence encoding a DNA/RNA-targeting IsrB polypeptide nuclease is codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In one embodiment, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In one embodiment, one or more codons (e.g., 1, 2, 3, 4, 5, 10, 15, 20, 25, 50, or more, or all codons) in a sequence encoding a IscB polypeptide nuclease corresponds to the most frequently used codon for a particular amino acid.

### Delivery

The present disclosure also provides delivery systems for introducing components of the systems and compositions herein to cells, tissues, organs, or organisms. A delivery system may comprise one or more delivery vehicles and/or cargos. Exemplary delivery systems and methods include those described in paragraphs to of Feng Zhang et al., (WO2016106236A1), and pages 1241-1251 and Table 1 of Lino C A et al., Delivering CRISPR: a review of the challenges and approaches, DRUG DELIVERY, 2018, VOL. 25, NO. 1, 1234-1257, which are incorporated by reference herein in their entireties and can be adapted for use with the IscB proteins disclosed herein.

In one embodiment, the delivery systems may be used to introduce the components of the systems and compositions to plant cells. For example, the components may be delivered to plant using electroporation, microinjection, aerosol beam injection of plant cell protoplasts, biolistic methods, DNA particle bombardment, and/or Agrobacterium-mediated transformation. Examples of methods and delivery systems for plants include those described in Fu et al., Transgenic Res. 2000 February; 9 (1): 11-9; Klein R M, et al., Biotechnology. 1992; 24:384-6; Casas A M et al., Proc Natl Acad Sci USA. 1993 Dec. 1; 90 (23): 11212-11216; and U.S. Pat. No. 5,563,055, Davey M R et al., Plant Mol Biol. 1989 September; 13 (3): 273-85, which are incorporated by reference herein in their entireties.

The example delivery compositions, systems, and methods described herein related to composition or IsrB polypeptide nuclease also apply to functional domains and other components (e.g., other proteins and polynucleotides related to the IsrB polypeptide nuclease, such as reverse transcriptase, nucleotide deaminase, retrotransposon, donor polynucleotide, etc.).

### Cargos

The delivery systems may comprise one or more cargos. The cargos may comprise one or more components of the systems and compositions herein. A cargo may comprise one or more of the following: i) a plasmid encoding one or more protein components in the compositions and systems such as the IsrB polypeptide nuclease and/or functional domains; ii) a plasmid encoding one or more ωRNAs, iii) mRNA of one or more one or more proteins components in the compositions and systems such as the IsrB polypeptide nuclease and/or functional domains; iv) one or more proteins components in the compositions and systems such as the IsrB polypeptide nuclease and/or functional domains; v) any combination thereof. The one or more protein components may include the nuclei acid-guided nuclease (e.g., Cas), reverse transcriptase, nucleotide deaminase, retrotransposon protein, other functional domain, or any combination thereof.

In some examples, a cargo may comprise a plasmid encoding one or more proteins components in the compositions and systems such as the IsrB polypeptide nuclease and/or functional domains and one or more (e.g., a plurality of) ωRNAs. In some cases, the plasmid may also encode a recombination template (e.g., for HDR). In one embodiment, a cargo may comprise mRNA encoding one or more protein components and one or more ωRNA.

In some examples, a cargo may comprise one or more protein components and one or more ωRNA, e.g., in the form of ribonucleoprotein complexes (RNP). The ribonucleoprotein complexes may be delivered by methods and systems herein. In some cases, the ribonucleoprotein may be delivered by way of a polypeptide-based shuttle agent. In one example, the ribonucleoprotein may be delivered using synthetic peptides comprising an endosome leakage domain (ELD) operably linked to a cell penetrating domain (CPD), to a histidine-rich domain and a CPD, e.g., as describe in WO2016161516. RNP may also be used for delivering the compositions and systems to plant cells, e.g., as described in Wu J W, et al., Nat Biotechnol. 2015 November; 33 (11): 1162-4.

### Physical Delivery

In one embodiment, the cargos may be introduced to cells by physical delivery methods. Examples of physical methods include microinjection, electroporation, and hydrodynamic delivery. Both nucleic acid and proteins may be delivered using such methods. For example, one or more protein components may be prepared in vitro, isolated, (refolded, purified if needed), and introduced to cells.

### Microinjection

Microinjection of the cargo directly to cells can achieve high efficiency, e.g., above 90% or about 100%. In one embodiment, microinjection may be performed using a microscope and a needle (e.g., with 0.5-5.0 μm in diameter) to pierce a cell membrane and deliver the cargo directly to a target site within the cell. Microinjection may be used for in vitro and ex vivo delivery.

Plasmids comprising coding sequences for one or more protein components and/or ωRNAs, mRNAs may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery ωRNA directly to the nucleus and mRNA to the cytoplasm, e.g., facilitating translation and shuttling of one or more protein components to the nucleus.

Microinjection may be used to generate genetically modified animals. For example, gene editing cargos may be injected into zygotes to allow for efficient germline modification. Such approach can yield normal embryos and full-term mouse pups harboring the desired modification(s). Microinjection can also be used to provide transiently up- or down-regulate a specific gene within the genome of a cell, e.g., using IsrB polypeptide.

### Electroporation

In one embodiment, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the cargos, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The cargos, e.g., nucleic acids, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses, non-viral vehicles, and other delivery reagents described herein.

The delivery vehicles in accordance with the present invention may have a greatest dimension (e.g. diameter) of less than 100 microns (μm). In one embodiment, the delivery vehicles have a greatest dimension of less than 10 μm. In one embodiment, the delivery vehicles may have a greatest dimension of less than 2000 nanometers (nm). In one embodiment, the delivery vehicles may have a greatest dimension of less than 1000 nanometers (nm). In one embodiment, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In one embodiment, the delivery vehicles may have a greatest dimension ranging between 25 nm and 200 nm.

In one embodiment, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles). Nanoparticles may also be used to deliver the compositions and systems to plant cells, e.g., as described in International Patent Publication No. WO 2008042156, US Publication Application No. US20130185823, and International Patent Publication No WO 2015/089419.

### Vectors

The systems, compositions, and/or delivery systems may comprise one or more vectors. The present disclosure also includes vector systems. A vector system may comprise one or more vectors. In one embodiment, a vector refers to a nucleic acid molecule capable of transporting another nucleic acid to which it has been linked. Vectors include nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. A vector may be a plasmid, e.g., a circular double stranded DNA loop into which additional DNA segments can be inserted, such as by standard molecular cloning techniques. Certain vectors may be capable of autonomous replication in a host cell into which they are introduced (e.g., bacterial vectors having a bacterial origin of replication and episomal mammalian vectors). Some vectors (e.g., non-episomal mammalian vectors) are integrated into the genome of a host cell upon introduction into the host cell, and thereby are replicated along with the host genome. In certain examples, vectors may be expression vectors, e.g., capable of directing the expression of genes to which they are operatively-linked. In some cases, the expression vectors may be for expression in eukaryotic cells. Common expression vectors of utility in recombinant DNA techniques are often in the form of plasmids.

Examples of vectors include pGEX, pMAL, pRIT5, E. coli expression vectors (e.g., pTrc, pET 11d, yeast expression vectors (e.g., pYepSec1, pMFa, pJRY88, pYES2, and picZ, Baculovirus vectors (e.g., for expression in insect cells such as SF9 cells) (e.g., pAc series and the pVL series), mammalian expression vectors (e.g., pCDM8 and pMT2PC.

A vector may comprise i) one or more protein components encoding sequence(s), and/or ii) a single, or at least 2, at least 3, at least 4, at least 5, at least 6, at least 7, at least 8, at least 9, at least 10, at least 12, at least 14, at least 16, at least 32, at least 48, at least 50 guide RNA(s) encoding sequences. In a single vector there can be a promoter for each RNA coding sequence. Alternatively or additionally, in a single vector, there may be a promoter controlling (e.g., driving transcription and/or expression) multiple RNA encoding sequences.

Furthermore, that compositions or systems may be delivered via a vector, e.g., a separate vector or the same vector that is encoding the complex. When provided by a separate vector, the RNA that targets IsrB polypeptide nuclease expression can be administered sequentially or simultaneously. When administered sequentially, the RNA that targets IsrB polypeptide nuclease expression is to be delivered after the RNA that is intended for e.g. gene editing or gene engineering. This period may be a period of minutes (e.g. 5 minutes, 10 minutes, 20 minutes, 30 minutes, 45 minutes, 60 minutes). This period may be a period of hours (e.g. 2 hours, 4 hours, 6 hours, 8 hours, 12 hours, 24 hours). This period may be a period of days (e.g. 2 days, 3 days, 4 days, 7 days). This period may be a period of weeks (e.g. 2 weeks, 3 weeks, 4 weeks). This period may be a period of months (e.g. 2 months, 4 months, 8 months, 12 months). This period may be a period of years (2 years, 3 years, 4 years). In this fashion, the IsrB polypeptide nuclease associates with a first hRNA molecule capable of hybridizing to a first target, such as a genomic locus or loci of interest and undertakes the function(s) desired of the system (e.g., gene engineering); and subsequently the IsrB polypeptide nuclease may then associate with the second hRNA molecule capable of hybridizing to the sequence comprising at least part of the IsrB polypeptide nuclease. Where the guide RNA targets the sequences encoding expression of the IsrB polypeptide nuclease, the enzyme becomes impeded and the system becomes self-inactivating. In the same manner, RNA that targets IsrB polypeptide nuclease expression applied via, for example liposome, lipofection, particles, microvesicles as explained herein, may be administered sequentially or simultaneously. Similarly, self-inactivation may be used for inactivation of one or more ωRNA used to target one or more targets.

### Regulatory Elements

A vector may comprise one or more regulatory elements. The regulatory element(s) may be operably linked to coding sequences of IsrB polypeptide nuclease, accessory proteins, and a ωRNA scaffold. The term “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). In certain examples, a vector may comprise: a first regulatory element operably linked to a nucleotide sequence encoding a IsrB polypeptide nuclease, and a second regulatory element operably linked to a nucleotide sequence encoding a ωRNA.

Examples of regulatory elements include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific.

Examples of promoters include one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter.

### Viral Vectors

The cargos may be delivered by viruses. In one embodiment, viral vectors are used. A viral vector may comprise virally-derived DNA or RNA sequences for packaging into a virus (e.g., retroviruses, replication defective retroviruses, adenoviruses, replication defective adenoviruses, and adeno-associated viruses). Viral vectors also include polynucleotides carried by a virus for transfection into a host cell. Viruses and viral vectors may be used for in vitro, ex vivo, and/or in vivo deliveries.

### Adeno Associated Virus (AAV)

The systems and compositions herein may be delivered by adeno associated virus (AAV). AAV vectors may be used for such delivery. AAV, of the Dependovirus genus and Parvoviridae family, is a single stranded DNA virus. In one embodiment, AAV may provide a persistent source of the provided DNA, as AAV delivered genomic material can exist indefinitely in cells, e.g., either as exogenous DNA or, with some modification, be directly integrated into the host DNA. In one embodiment, AAV do not cause or relate with any diseases in humans. The virus itself is able to efficiently infect cells while provoking little to no innate or adaptive immune response or associated toxicity.

Examples of AAV that can be used herein include AAV-1, AAV-2, AAV-3, AAV-4, AAV-5, AAV-6, AAV-8, and AAV-9. The type of AAV may be selected with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. AAV-2-based vectors were originally proposed for CFTR delivery to CF airways, other serotypes such as AAV-1, AAV-5, AAV-6, and AAV-9 exhibit improved gene transfer efficiency in a variety of models of the lung epithelium. Examples of cell types targeted by AAV are described in Grimm, D. et al, J. Virol. 82:5887-5911 (2008)), and shown in Table 5 as follows:

TABLE 3
Examples of cell types targeted by AAV.

| | AAV- | AAV- | AAV- | AAV- | AAV- | AAV- | AAV- | AAV- |
|---|---|---|---|---|---|---|---|---|
| Cell Line | 1 | 2 | 3 | 4 | 5 | 6 | 8 | 9 |
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature | 2500 | 100 | ND | ND | 222 | 2857 | ND | ND |
| DC | | | | | | | | |
| Mature DC | 2222 | 100 | ND | ND | 333 | 3333 | ND | ND |

The AAV particles may be created in HEK 293 T cells. Once particles with specific tropism have been created, they are used to infect the target cell line much in the same way that native viral particles do. This may allow for persistent presence of the components in the infected cell type, and what makes this version of delivery particularly suited to cases where long-term expression is desirable. Examples of doses and formulations for AAV that can be used include those describe in U.S. Pat. Nos. 8,454,972 and 8,404,658.

Various strategies may be used for delivery the systems and compositions herein with AAVs. In some examples, coding sequences of IsrB polypeptide nuclease and @ RNA may be packaged directly onto one DNA plasmid vector and delivered via one AAV particle. In some examples, AAVs may be used to deliver ωRNAs into cells that have been previously engineered to express IsrB polypeptide nuclease. In some examples, coding sequences of IsrB polypeptide nuclease and ωRNA may be made into two separate AAV particles, which are used for co-transfection of target cells. In some examples, markers, tags, and other sequences may be packaged in the same AAV particles as coding sequences of IsrB polypeptide nuclease and/or @ RNAs.

### Lentiviruses

The systems and compositions herein may be delivered by lentiviruses. Lentiviral vectors may be used for such delivery. Lentiviruses are complex retroviruses that have the ability to infect and express their genes in both mitotic and post-mitotic cells.

Examples of lentiviruses include human immunodeficiency virus (HIV), which may use its envelope glycoproteins of other viruses to target a broad range of cell types; minimal non-primate lentiviral vectors based on the equine infectious anemia virus (EIAV), which may be used for ocular therapies. In an embodiment, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) may be used/and or adapted to the nucleic acid-targeting system herein.

Lentiviruses may be pseudo-typed with other viral proteins, such as the G protein of vesicular stomatitis virus. In doing so, the cellular tropism of the lentiviruses can be altered to be as broad or narrow as desired. In some cases, to improve safety, second- and third-generation lentiviral systems may split essential genes across three plasmids, which may reduce the likelihood of accidental reconstitution of viable viral particles within cells.

In some examples, leveraging the integration ability, lentiviruses may be used to create libraries of cells comprising various genetic modifications, e.g., for screening and/or studying genes and signaling pathways.

### Adenoviruses

The systems and compositions herein may be delivered by adenoviruses. Adenoviral vectors may be used for such delivery. Adenoviruses include nonenveloped viruses with an icosahedral nucleocapsid containing a double stranded DNA genome. Adenoviruses may infect dividing and non-dividing cells. In one embodiment, adenoviruses do not integrate into the genome of host cells, which may be used for limiting off-target effects of systems in gene editing applications.

### Viral Vehicles for Delivery to Plants

The systems and compositions may be delivered to plant cells using viral vehicles. In one embodiment, the compositions and systems may be introduced in the plant cells using a plant viral vector (e.g., as described in Scholthof et al. 1996, Annu Rev Phytopathol. 1996; 34:299-323). Such viral vector may be a vector from a DNA virus, e.g., geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). The viral vector may be a vector from an RNA virus, e.g., tobravirus (e.g., tobacco rattle virus, tobacco mosaic virus), potexvirus (e.g., potato virus X), or hordeivirus (e.g., barley stripe mosaic virus). The replicating genomes of plant viruses may be non-integrative vectors.

### Non-Viral Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, gold nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes.

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of IscB polypeptide nuclease and/or hRNA) and/or RNA molecules (e.g., mRNA of IsrB polypeptide nuclease, ωRNA). In certain cases, LNPs may be use for delivering RNP complexes of IsrB polypeptide/@ RNA.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-O-[2″-(methoxypolyethyleneglycol 2000) succinoyl]-1,2-dimyristoyl-sn-glycol (PEG-S-DMG), R-3-[(ro-methoxy-poly(ethylene glycol) 2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-220 Dec. 2011).

### Liposomes

In one embodiment, a lipid particle may be liposome. Liposomes are spherical vesicle structures composed of a uni- or multilamellar lipid bilayer surrounding internal aqueous compartments and a relatively impermeable outer lipophilic phospholipid bilayer. In one embodiment, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

### Stable Nucleic-Acid-Lipid Particles (SNALPs)

In one embodiment, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol) 2000) carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-CDMA, and 1,2-dilinoleyloxy-3-(N;N-dimethyl)aminopropane (DLinDMA).

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

### Lipoplexes/Polyplexes

In one embodiment, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2p (e.g., forming DNA/Ca²⁺ microcomplexes), polyethenimine (PEI) (e.g., branched PEI), and poly (L-lysine) (PLL).

### Cell Penetrating Peptides

In one embodiment, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin β3 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the IscB polypeptide nuclease directly, which is then complexed with the hRNA and delivered to cells. In some examples, separate delivery of CPP-IscB and CPP-hRNA to multiple cells may be performed. CPP may also be used to delivery RNPs.

CPPs may be used to deliver the compositions and systems to plants. In some examples, CPPs may be used to deliver the components to plant protoplasts, which are then regenerated to plant cells and further to plants.

### Dna Nanoclews

In one embodiment, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136 (42): 14722-5; and Sun W et al, Angew Chem Int Ed Engl. 2015 Oct. 5; 54 (41): 12029-33. DNA nanoclew may have a palindromic sequences to be partially complementary to the guide RNA within the IscB polypeptide nuclease: hRNA ribonucleoprotein complex. A DNA nanoclew may be coated, e.g., coated with PEI to induce endosomal escape.

### Gold Nanoparticles

In one embodiment, the delivery vehicles comprise gold nanoparticles (also referred to AuNPs or colloidal gold). Gold nanoparticles may form complex with cargos, e.g., IscB polypeptide nuclease: hRNA RNP. Gold nanoparticles may be coated, e.g., coated in a silicate and an endosomal disruptive polymer, PAsp (DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901.

iTOP

In one embodiment, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D′Astolfo D S, Pagliero R J, Pras A, et al. (2015). Cell 161:674-690.

### Polymer-Based Particles

In one embodiment, the delivery vehicles may comprise polymer-based particles (e.g., nanoparticles). In one embodiment, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In one embodiment, the polymer-based particles may comprise alkylated and carboxyalkylated branched polyethylenimine. In some examples, the polymer-based particles are VIROMER, e.g., VIROMER RNAi, VIROMER RED, VIROMER mRNA. Example methods of delivering the systems and compositions herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection-Factbook 2018: technology, product overview, users' data., doi: 10.13140/RG.2.2.23912.16642.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6: e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In one embodiment, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267 (1): 9-21; El-Andaloussi S, et al., Nat Protoc. 2012 December; 7 (12): 2112-26; Uno Y, et al., Hum Gene Ther. 2011 June; 22 (6): 711-9; Zou W, et al., Hum Gene Ther. 2011 April; 22 (4): 465-75.

In some examples, the exosome may form a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427h.

### Retrovirus Like Delivery Systems

The delivery vehicle may comprise a retro-virus like protein, such as PEG10, which is capable of incorporating a cargo into a virus-like particle. As such systems can be re-programmed to package specific cargos, polynucleotides encoding composnents of the IsrB systems disclosed herein may be further modified with a recognition sequence that leads to selective packaging of the IsrB components into such retro-virus like VLPs. Said VLPs may be further modified with fusogenic proteins that impart tissue or cell specificity. Example systems are disclosed in Segal et al. Mammalian retrovirus-like protein PEG10 packages its own mRNA and can be pseudotyped for mRNA delivery. 373 Science, 882-889 (2021), which is incorporated herein by reference.

### Genetically Modified Cells and Organisms

The present disclosure further provides cells comprising one or more components of the compositions and systems herein, e.g., the IscB polypeptide or CRISPR-associated IscB polypeptide nuclease and/or ωRNA(s). Also provided include cells modified by the systems and methods herein, and cell cultures, tissues, organs, organism comprising such cells or progeny thereof. In one embodiment, the present disclosure provides a method of modifying an cell or organism. The cell may be a prokaryotic cell or a eukaryotic cell. The cell may be a mammalian cell. The mammalian cell many be a non-human primate, bovine, porcine, rodent or mouse cell. The cell may be a non-mammalian eukaryotic cell such as poultry, fish or shrimp. The cell may be a therapeutic T cell or antibody-producing B-cell. The cell may also be a plant cell. The plant cell may be of a crop plant such as cassava, corn, sorghum, wheat, or rice. The plant cell may also be of an algae, tree or vegetable. The modification introduced to the cell by the present invention may be such that the cell and progeny of the cell are altered for improved production of biologic products such as an antibody, starch, alcohol or other desired cellular output. The modification introduced to the cell by the present invention may be such that the cell and progeny of the cell include an alteration that changes the biologic product produced.

In one embodiment, one or more polynucleotide molecules, vectors, or vector systems driving expression of one or more elements of the compositions, systems, or delivery systems comprising one or more elements of the nucleic acid-targeting system are introduced into a host cell such that expression of the elements of the nucleic acid-targeting system direct formation of a nucleic acid-targeting complex at one or more target sites. In an embodiment of the invention the host cell may be a eukaryotic cell, a prokaryotic cell, or a plant cell.

In one embodiment, the host cell is a cell of a cell line. Cell lines are available from a variety of sources known to those with skill in the art (see, e.g., the American Type Culture Collection (ATCC) (Manassas, Va.)). In one embodiment, a cell transfected with one or more vectors described herein is used to establish a new cell line comprising one or more vector-derived sequences. In one embodiment, a cell transiently transfected with the components of a system as described herein (such as by transient transfection of one or more vectors, or transfection with RNA), and modified through the activity of a complex, is used to establish a new cell line comprising cells containing the modification but lacking any other exogenous sequence. In one embodiment, cells transiently or non-transiently transfected with one or more vectors described herein, or cell lines derived from such cells are used in assessing one or more test compounds.

Further intended are isolated human cells or tissues, plants or non-human animals comprising one or more of the polynucleotide molecules, vectors, vector systems, or cells described in any of the embodiments herein. In an aspect, host cells and cell lines modified by or comprising the compositions, systems or modified enzymes of present invention are provided, including (isolated) stem cells, and progeny thereof.

In an embodiment, the plants or non-human animals comprise at least one of the system components, polynucleotide molecules, vectors, vector systems, or cells described in any of the embodiments herein at least one tissue type of the plant or non-human animal. In an embodiment, non-human animals comprise at least one of the system components, polynucleotide molecules, vectors, vector systems, or cells described in any of the embodiments herein in at least one tissue type. In an embodiment, the presence of the system components is transient, in that they are degraded over time. In an embodiment, expression of the components of the systems and compositions described in any of the embodiments comprised in polynucleotide molecules, vectors, vector systems, or cells is limited to certain tissue types or regions in the plant or non-human animal. In an embodiment, the expression of the components of the systems and compositions described in any of the embodiments comprised in polynucleotide molecules, vectors, vector systems, or cells is dependent of a physiological cue. In an embodiment, expression of the components of the systems and compositions described in any of the embodiments comprised in polynucleotide molecules, vectors, vector systems, or cells may be triggered by an exogenous molecule. In an embodiment, expression of the components of the systems and compositions described in any of the embodiments comprised in polynucleotide molecules, vectors, vector systems, or cells is dependent on the expression of a non-cas molecule in the plant or non-human animal.

### Applications and Uses in General

The systems, the vector systems, the vectors and the compositions described herein may be used in various nucleic acids-targeting applications, altering or modifying synthesis of a gene product, such as a protein, nucleic acids cleavage, nucleic acids editing, nucleic acids splicing; trafficking of target nucleic acids, tracing of target nucleic acids, isolation of target nucleic acids, visualization of target nucleic acids, etc.

Aspects of the invention thus also encompass methods and uses of the compositions and systems described herein in genome engineering, e.g. for altering or manipulating the expression of one or more genes or the one or more gene products, in prokaryotic or eukaryotic cells, in vitro, in vivo or ex vivo. In some examples, the target polynucleotides are target sequences within genomic DNA, including nuclear genomic DNA, mitochondrial DNA, or chloroplast DNA.

Typically, in the context of a nucleic acid-targeting system, formation of a nucleic acid-targeting complex (comprising a ωRNA or guide RNA hybridized to a target sequence and complexed with one or more nucleic acid-targeting effector proteins) results in cleavage of one or both DNA or RNA strands in or near (e.g., within 1, 2, 3, 4, 5, 6, 7, 8, 9, 10, 20, 50, or more base pairs from) the target sequence. As used herein the term “sequence(s) associated with a target locus of interest” refers to sequences near the vicinity of the target sequence (e.g. within 1, 2, 3, 4, 5, 6, 7, 8, 9, 10, 20, 50, or more base pairs from the target sequence, wherein the target sequence is comprised within a target locus of interest).

In one embodiment, the present disclosure provides a method of targeting a polynucleotide, comprising contacting a sample (such as cell, population of cells, tissue, organ, or an organism) that comprises a target polynucleotide with the composition, systems, polynucleotide(s), or vector(s). The contacting may result in modification of a gene product or modification of the amount or expression of a gene product. In some examples, the target sequence of the polynucleotide is a disease-associated target sequence.

In one embodiment, the present disclosure provides a method of modifying target polynucleotides comprising delivering the composition, the one or more polynucleotides of 2, or one or more vectors to a cell or population of cells comprising the target polynucleotides, wherein the complex directs the reverse transcriptase to the target sequence and the reverse transcriptase facilitates insertion of the donor sequence from the ωRNA into the target polynucleotide.

Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

The target polynucleotide of a complex can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA). Without wishing to be bound by theory, it is believed that the target sequence should be associated with a TAM (target adjacent motif); that is, a short sequence recognized by the complex. The precise sequence and length requirements for the TAM differ depending on the IsrB polypeptide nuclease used, but TAMs are typically 2-5 base pair sequences adjacent the protospacer (that is, the target sequence) A skilled person will be able to identify further TAM sequences for use with a given IsrB polypeptide nuclease. Further, engineering of the TAM Interacting domain may allow programing of TAM specificity, improve target site recognition fidelity, and increase the versatility of the IsrB polypeptide nuclease, genome engineering platform. IsrB polypeptide nuclease may be engineered to alter their TAM specificity.

In an embodiment the IsrB TAM is ATNA where N is any nucleotide. In an embodiment, the IsrB TAM is ATGA, ATAA, ATAAA, or ATN. In one embodiment, the IsrB is Ignatius tetrasporus and the TAM is NNG.

Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

Aspects of the invention relate to a method of targeting a polynucleotide, comprising contacting a sample that comprises the polynucleotide with a composition, system or IsrB polypeptide nuclease as described in any embodiment herein, a delivery system comprising a composition, system or IsrB polypeptide nuclease as described in any embodiment herein, a polynucleotide comprising a composition, system or IsrB polypeptide nuclease as described in any embodiment herein, a vector comprising a composition, system or IsrB polypeptide nuclease as described in any embodiment herein, or a vector system comprising a composition, system or IsrB polypeptide nuclease as described in any embodiment herein. In an embodiment, a target polynucleotide is contacted with at least two different composition, system or IsrB nucleases. In further embodiments, the two different IsrB polypeptide nuclease have different target polynucleotide specificities, or degrees of specificity. In an embodiment, the two different IsrB polypeptide nuclease have a different TAM specificity.

Also envisaged are methods of targeting a polynucleotide, comprising contacting a sample that comprises the polynucleotide with the composition and systems, vectors, polynucleotides, herein wherein contacting results in modification of a gene product or modification of the amount or expression of a gene product. In an embodiment, the expression of the targeted gene product is increased by the method. In an embodiment, the expression of the targeted gene product is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, p at least 90%, at least 95%, 100%. In an embodiment, the expression of the targeted gene product is increased at least 1.5-fold, at least 2-fold, at least 2.5-fold, at least 3-fold, at least 3.5-fold, at least 3.5-fold, at least 4-fold, at least 4.5-fold, at least 5-fold, at least 10-fold, at least 10-fold, at least 15-fold, at least 20-fold, at least 25-fold, at least 50-fold, at least 100-fold. In an embodiment, the expression of the targeted gene product is reduced by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%. In an embodiment, the expression of the targeted gene product is reduced at least 1.5-fold, at least 2-fold, at least 2.5-fold, at least 3-fold, at least 3.5-fold, at least 3.5-fold, at least 4-fold, at least 4.5-fold, at least 5-fold, at least 10-fold, at least 10-fold, at least 15-fold, at least 20-fold, at least 25-fold, at least 50-fold, at least 100-fold. In alternative embodiments, the expression of the targeted gene product is reduced by the method. In further embodiments, expression of the targeted gene may be completely eliminated, or may be considered eliminated as remnant expression levels of the targeted gene fall below the detection limit of methods known in the art that are used to quantify, detect, or monitor expression levels of genes.

In one embodiment, one or more polynucleotide molecules, vectors, or vector systems driving expression of one or more elements of a nucleic acid-targeting system or delivery systems comprising one or more elements of the nucleic acid-targeting system are introduced into a host cell such that expression of the elements of the nucleic acid-targeting system direct formation of a nucleic acid-targeting complex at one or more target sites. In an embodiment of the invention the host cell may be a eukaryotic cell, a prokaryotic cell, or a plant cell.

In one embodiment, the host cell is a cell of a cell line. Cell lines are available from a variety of sources known to those with skill in the art (see, e.g., the American Type Culture Collection (ATCC) (Manassas, Va.)). In one embodiment, a cell transfected with one or more vectors described herein is used to establish a new cell line comprising one or more vector-derived sequences. In one embodiment, a cell transiently transfected with the components of a composition or system as described herein (such as by transient transfection of one or more vectors, or transfection with RNA), and modified through the activity of a complex, is used to establish a new cell line comprising cells containing the modification but lacking any other exogenous sequence. In one embodiment, cells transiently or non-transiently transfected with one or more vectors described herein, or cell lines derived from such cells are used in assessing one or more test compounds.

Further intended are isolated human cells or tissues, plants or non-human animals comprising one or more of the polynucleotide molecules, vectors, vector systems, or cells described in any of the embodiments herein. In an aspect, host cells and cell lines modified by or comprising the compositions, systems or modified enzymes of present invention are provided, including (isolated) stem cells, and progeny thereof.

In an embodiment, the plants or non-human animals comprise at least one of the compositions, polynucleotide molecules, vectors, vector systems, or cells described in any of the embodiments herein at least one tissue type of the plant or non-human animal. In certain embodiment, non-human animals comprise at least one of the compositions, polynucleotide molecules, vectors, vector systems, or cells described in any of the embodiments herein in at least one tissue type. In an embodiment, the presence of the compositions is transient, in that they are degraded over time. In an embodiment, expression of the compositions described in any of the embodiments comprised in polynucleotide molecules, vectors, vector systems, or cells is limited to certain tissue types or regions in the plant or non-human animal. In an embodiment, the expression of the compositions described in any of the embodiments comprised in polynucleotide molecules, vectors, vector systems, or cells is dependent of a physiological cue. In an embodiment, expression of the compositions described in any of the embodiments comprised in polynucleotide molecules, vectors, vector systems, or cells may be triggered by an exogenous molecule. In an embodiment, expression of the compositions described in any of the embodiments comprised in polynucleotide molecules, vectors, vector systems, or cells is dependent on the expression of a non-Cas molecule in the plant or non-human animal.

In one aspect, the invention provides methods for using one or more elements of a nucleic acid-targeting system. The nucleic acid-targeting complex of the invention provides an effective means for modifying a target DNA or RNA (single or double stranded, linear or super-coiled). The nucleic acid-targeting complex of the invention has a wide variety of utility including modifying (e.g., deleting, inserting, translocating, inactivating, activating) a target DNA or RNA in a multiplicity of cell types. As such, the nucleic acid-targeting complex of the invention has a broad spectrum of applications in, e.g., gene therapy, drug screening, disease diagnosis, and prognosis. An exemplary nucleic acid-targeting complex comprises a DNA or RNA-targeting effector protein complexed with a ωRNA hybridized to a target sequence within the target locus of interest.

In one embodiment, this invention provides a method of cleaving a target polynucleotide. The method may comprise modifying a target polynucleotide using a nucleic acid-targeting complex that binds to the target polynucleotide and effect cleavage of said target polynucleotide. In an embodiment, the nucleic acid-targeting complex of the invention, when introduced into a cell, may create a break (e.g., a single strand break) in the polynucleotide sequence. For example, the method can be used to cleave a disease polynucleotide in a cell. For example, an exogenous template comprising a sequence to be integrated flanked by an upstream sequence and a downstream sequence may be introduced into a cell. The upstream and downstream sequences share sequence similarity with either side of the site of integration in the polynucleotide. The exogenous template comprises a sequence to be integrated (e.g., a mutated RNA). The sequence for integration may be a sequence endogenous or exogenous to the cell. Examples of a sequence to be integrated include polynucleotide encoding a protein or a non-coding RNA (e.g., a microRNA). Thus, the sequence for integration may be operably linked to an appropriate control sequence or sequences. Alternatively, the sequence to be integrated may provide a regulatory function. The upstream and downstream sequences in the recombination template are selected to promote recombination between the RNA sequence of interest and the recombination. The upstream sequence is a polynucleotide sequence that shares sequence similarity with the sequence upstream of the targeted site for integration. Similarly, the downstream sequence is a polynucleotide sequence that shares sequence similarity with the polynucleotide sequence downstream of the targeted site of integration. The upstream and downstream sequences in the recombination template can have 75%, 80%, 85%, 90%, 95%, or 100% sequence identity with the targeted sequence. Preferably, the upstream and downstream sequences in the recombination template have about 95%, 96%, 97%, 98%, 99%, or 100% sequence identity with the targeted sequence. In some methods, the upstream and downstream sequences in the recombination template have about 99% or 100% sequence identity with the targeted sequence. An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000 bp. In some methods, the recombination template may further comprise a marker. Such a marker may make it easy to screen for targeted integrations. Examples of suitable markers include restriction sites, fluorescent proteins, or selectable markers. The recombination template of the invention can be constructed using recombinant techniques (see, for example, Sambrook et al., 2001 and Ausubel et al., 1996). In a method for modifying a target sequence by integrating an recombination template, a break (e.g., double or single stranded break in double or single stranded DNA or RNA) is introduced into the DNA or RNA sequence by the nucleic acid-targeting complex, the break is repaired via homologous recombination with an recombination template such that the template is integrated into the target. The presence of a double-stranded break facilitates integration of the template. In other embodiments, this invention provides a method of modifying expression of a RNA in a eukaryotic cell. The method comprises increasing or decreasing expression of a target polynucleotide by using a nucleic acid-targeting complex that binds to the DNA or RNA (e.g., mRNA or pre-mRNA). In some methods, a target can be inactivated to affect the modification of the expression in a cell. For example, upon the binding of a nucleic acid-targeting complex to a target sequence in a cell, the target is inactivated such that the sequence is not translated, the coded protein is not produced, or the sequence does not function as the wild-type sequence does. For example, a protein or microRNA coding sequence may be inactivated such that the protein or microRNA or pre-microRNA transcript is not produced. The target of a nucleic acid-targeting complex can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., ncRNA, lncRNA, tRNA, or rRNA). Examples of target RNA include a sequence associated with a signaling biochemical pathway, e.g., a signaling biochemical pathway-associated polynucleotide. Examples of target polynucleotide include a disease associated polynucleotide. A “disease-associated” polynucleotide refers to any polynucleotide which is yielding translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated polynucleotide also refers to a gene possessing mutation(s) or genetic variation that is directly responsible or is in linkage disequilibrium with a gene(s) that is responsible for the etiology of a disease. The translated products may be known or unknown, and may be at a normal or abnormal level. The target RNA of a nucleic acid-targeting complex can be any polynucleotide endogenous or exogenous to the eukaryotic cell. For example, the target RNA can be a RNA residing in the nucleus of the eukaryotic cell. The target polynucleotide can be a sequence coding a gene product (e.g., a protein) or a non-coding sequence (e.g., ncRNA, lncRNA, tRNA, or rRNA).

In one embodiment, the method may comprise allowing a composition to bind to the target DNA or RNA to effect cleavage of said target DNA or RNA thereby modifying the target DNA or RNA, wherein the nucleic acid-targeting complex comprises a nucleic acid-targeting effector protein complexed with a ωRNA hybridized to a target sequence within said target DNA or RNA. In one aspect, the invention provides a method of modifying expression of DNA or RNA in a eukaryotic cell. In one embodiment, the method comprises allowing a nucleic acid-targeting complex to bind to the DNA or RNA such that said binding results in increased or decreased expression of said DNA or RNA; wherein the nucleic acid-targeting complex comprises a nucleic acid-targeting effector protein complexed with a ωRNA. Similar considerations and conditions apply as above for methods of modifying a target DNA or RNA. In fact, these sampling, culturing and re-introduction options apply across the aspects of the present invention. In one aspect, the invention provides for methods of modifying a target DNA or RNA in a eukaryotic cell, which may be in vivo, ex vivo or in vitro. In one embodiment, the method comprises sampling a cell or population of cells from a human or non-human animal, and modifying the cell or cells. Culturing may occur at any stage ex vivo. The cell or cells may even be re-introduced into the non-human animal or plant. For re-introduced cells it is particularly preferred that the cells are stem cells. The compositions as described in any embodiment herein may be used to detect nucleic acid identifiers. Nucleic acid identifiers are non-coding nucleic acids that may be used to identify a particular article. Example nucleic acid identifiers, such as DNA watermarks, are described in Heider and Barnekow. “DNA watermarks: A proof of concept” BMC Molecular Biology 9:40 (2008). The nucleic acid identifiers may also be a nucleic acid barcode. A nucleic-acid based barcode is a short sequence of nucleotides (for example, DNA, RNA, or combinations thereof) that is used as an identifier for an associated molecule, such as a target molecule and/or target nucleic acid. A nucleic acid barcode can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. One or more nucleic acid barcodes can be attached, or “tagged,” to a target molecule and/or target nucleic acid. This attachment can be direct (for example, covalent or non-covalent binding of the barcode to the target molecule) or indirect (for example, via an additional molecule, for example, a specific binding agent, such as an antibody (or other protein) or a barcode receiving adaptor (or other nucleic acid molecule). Target molecule and/or target nucleic acids can be labeled with multiple nucleic acid barcodes in combinatorial fashion, such as a nucleic acid barcode concatemer. Typically, a nucleic acid barcode is used to identify target molecules and/or target nucleic acids as being from a particular compartment (for example a discrete volume), having a particular physical property (for example, affinity, length, sequence, etc.), or having been subject to certain treatment conditions. Target molecule and/or target nucleic acid can be associated with multiple nucleic acid barcodes to provide information about all of these features (and more). Methods of generating nucleic acid-barcodes are disclosed, for example, in International Patent Application Publication No. WO/2014/047561.

In an embodiment, compositions induce a single strand break for the purpose of inducing HDR-mediated correction. In a further embodiment, two or more guide RNAs complexing with IsrB polypeptide nuclease or an ortholog or homolog thereof, may be used to induce multiplexed breaks for purpose of inducing HDR-mediated correction.

A recombination template nucleic acid, as that term is used herein, refers to a nucleic acid sequence which can be used in conjunction with compositions discloser herein to alter the structure of a target position. In an embodiment, the target nucleic acid is modified to have some or all of the sequence of the recombination template nucleic acid, typically at or near cleavage site(s). In an embodiment, the recombination template nucleic acid is single stranded. In an alternate embodiment, the recombination template nucleic acid is double stranded. In an embodiment, the recombination template nucleic acid is DNA, e.g., double stranded DNA. In an alternate embodiment, the recombination template nucleic acid is single stranded DNA.

In one embodiment, a recombination template is provided to serve as a template in homologous recombination, such as within or near a target sequence nicked or cleaved by a nucleic acid-targeting effector protein as a part of a nucleic acid-targeting complex.

A recombination template may be a component of another vector as described herein, contained in a separate vector, or provided as a separate polynucleotide. A recombination template polynucleotide may be of any suitable length, such as about or more than about 10, 15, 20, 25, 50, 75, 100, 150, 200, 500, 1000, or more nucleotides in length. In one embodiment, the recombination template polynucleotide is complementary to a portion of a polynucleotide comprising the target sequence. When optimally aligned, a recombination template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In one embodiment, when a recombination template sequence and a polynucleotide comprising a target sequence are optimally aligned, the nearest nucleotide of the recombination template polynucleotide is within about 1, 5, 10, 15, 20, 25, 50, 75, 100, 200, 300, 400, 500, 1000, 5000, 10000, or more nucleotides from the target sequence.

In an embodiment, the recombination template nucleic acid alters the structure of the target position by participating in homologous recombination. In an embodiment, the recombination template nucleic acid alters the sequence of the target position. In an embodiment, the recombination template nucleic acid results in the incorporation of a modified, or non-naturally occurring base into the target nucleic acid.

The recombination template sequence may undergo a breakage mediated or catalyzed recombination with the target sequence. In an embodiment, the recombination template nucleic acid may include sequence that corresponds to a site on the target sequence that is cleaved by an IsrB polypeptide nuclease mediated cleavage event. In an embodiment, the recombination template nucleic acid may include sequence that corresponds to both, a first site on the target sequence that is cleaved in a first IsrB polypeptide nuclease mediated event and a second site on the target sequence that is cleaved in a second IsrB polypeptide nuclease mediated event.

In an embodiment, the recombination template nucleic acid can include sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In an embodiment, the recombination template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A recombination template nucleic acid having homology with a target position in a target gene may be used to alter the structure of a target sequence. The recombination template sequence may be used to alter an unwanted structure, e.g., an unwanted or mutant nucleotide. The recombination template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The recombination template nucleic acid may include sequence which results in: a change in sequence of 1, 2, 3, 4, 5, 6, 7, 8, 9, 10, 1 1, 12 or more nucleotides of the target sequence. In an embodiment, the recombination template nucleic acid may be 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 1 80+/−10, 190+/−10, 200+/−10, 210+/−10, of 220+/−10 nucleotides in length. In an embodiment, the t recombination template nucleic acid may be 30+/−20, 40+/−20, 50+/−20, 60+/−20, 70+/−20, 80+/−20, 90+/−20, 100+/−20, 1 10+/−20, 120+/−20, 130+/−20, 140+/−20, I 50+/−20, 160+/−20, 170+/−20, 180+/−20, 190+/−20, 200+/−20, 210+/−20, of 220+/−20 nucleotides in length. In an embodiment, the recombination template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

A recombination template nucleic acid comprises the following components: [5′ homology arm]-[replacement sequence]-[3′ homology arm]. The homology arms provide for recombination into the chromosome, thus replacing the undesired element, e.g., a mutation or signature, with the replacement sequence. In an embodiment, the homology arms flank the most distal cleavage sites. In an embodiment, the 3′ end of the 5′ homology arm is the position next to the 5′ end of the replacement sequence. In an embodiment, the 5′ homology arm can extend at least 10, 20, 30, 40, 50, 100, 200, 300, 400, 500, 600, 700, 800, 900, 1000, 1500, or 2000 nucleotides 5′ from the 5′ end of the replacement sequence. In an embodiment, the 5′ end of the 3′ homology arm is the position next to the 3′ end of the replacement sequence. In an embodiment, the 3′ homology arm can extend at least 10, 20, 30, 40, 50, 100, 200, 300, 400, 500, 600, 700, 800, 900, 1000, 1500, or 2000 nucleotides 3′ from the 3′ end of the replacement sequence.

In an embodiment, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In one embodiment, both the 5′ and the 3′ homology arms may be shortened to avoid including certain sequence repeat elements.

In an embodiment, a recombination template nucleic acids for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Unlike IsrB polypeptide nuclease-mediated gene knockout, which permanently eliminates expression by mutating the gene at the DNA level, IsrB polypeptide nuclease knockdown allows for temporary reduction of gene expression through the use of artificial transcription factors. Mutating key residues in both DNA cleavage domains of the IsrB polypeptide nuclease, results in the generation of a catalytically inactive IsrB polypeptide nuclease. A catalytically inactive IsrB polypeptide nuclease complexes with a ωRNA and localizes to the DNA sequence specified by that guide RNA's targeting domain, however, it does not cleave the target DNA. Fusion of the inactive IsrB polypeptide nuclease protein to an effector domain, e.g., a transcription repression domain, enables recruitment of the effector to any DNA site specified by the ωRNA. In an embodiment, IsrB polypeptide nuclease may be fused to a transcriptional repression domain and recruited to the promoter region of a gene. Especially for gene repression, it is contemplated herein that blocking the binding site of an endogenous transcription factor would aid in downregulating gene expression. In another embodiment, an inactive IsrB polypeptide nuclease can be fused to a chromatin modifying protein. Altering chromatin status can result in decreased expression of the target gene.

In an embodiment, a ωRNA molecule can be targeted to a known transcription response elements (e.g., promoters, enhancers, etc.), a known upstream activating sequences, and/or sequences of unknown or known function that are suspected of being able to control expression of the target DNA.

In some methods, a target polynucleotide can be inactivated to affect the modification of the expression in a cell. For example, upon the binding of a composition to a target sequence in a cell, the target polynucleotide is inactivated such that the sequence is not transcribed, the coded protein is not produced, or the sequence does not function as the wild-type sequence does. For example, a protein or microRNA coding sequence may be inactivated such that the protein is not produced.

### Non-Homologous End-Joining

In an embodiment, nuclease-induced non-homologous end-joining (NHEJ) can be used to target gene-specific knockouts. Nuclease-induced NHEJ can also be used to remove (e.g., delete) sequence in a gene of interest. Generally, NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. Two-thirds of these mutations typically alter the reading frame and, therefore, produce a non-functional protein. Additionally, mutations that maintain the reading frame, but which insert or delete a significant amount of sequence, can destroy functionality of the protein. This is locus dependent as mutations in critical functional domains are likely less tolerable than mutations in non-critical regions of the protein. The indel mutations generated by NHEJ are unpredictable in nature; however, at a given break site certain indel sequences are favored and are over-represented in the population, likely due to small regions of microhomology. The lengths of deletions can vary widely; most commonly in the 1-50 bp range, but they can easily be greater than 50 bp, e.g., they can easily reach greater than about 100-200 bp. Insertions tend to be shorter and often include short duplications of the sequence immediately surrounding the break site. However, it is possible to obtain large insertions, and in these cases, the inserted sequence has often been traced to other regions of the genome or to plasmid DNA present in the cells.

Because NHEJ is a mutagenic process, it may also be used to delete small sequence motifs as long as the generation of a specific final sequence is not required. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences; however, the error-prone nature of NHEJ may still produce indel mutations at the site of repair.

Both double strand cleaving IscB polypeptide nuclease, or an ortholog or homolog thereof, and single strand, or nickase, IscB polypeptide nuclease, or an ortholog or homolog thereof, molecules can be used in the methods and compositions described herein to generate NHEJ-mediated indels. NHEJ-mediated indels targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp).

In an embodiment, in which a ωRNA and IsrB polypeptide nuclease, or an ortholog or homolog thereof, generate a single strand break for the purpose of inducing NHEJ-mediated indels, a ωRNA may be configured to position one double-strand break in close proximity to a nucleotide of the target position. In an embodiment, the cleavage site may be between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position).

In an embodiment, in which two ωRNAs complexing with IsrB polypeptide nuclease, or an ortholog or homolog thereof, e.g., IsrB polypeptide nickases induce two single strand breaks for the purpose of inducing NHEJ-mediated indels, two ωRNAs may be configured to position two single-strand breaks to provide for NHEJ repair a nucleotide of the target position.

In some examples, the systems herein may introduce one or more indels via NHEJ pathway and insert sequence from a combination template via HDR.

### Exemplary Applications

The invention provides a non-naturally occurring or engineered composition, or one or more polynucleotides encoding components of said composition, or vector or delivery systems comprising one or more polynucleotides encoding components of said composition for use in a modifying a target cell in vivo, ex vivo or in vitro and, may be conducted in a manner alters the cell such that once modified the progeny or cell line of the IsrB polypeptide nuclease modified cell retains the altered phenotype. The modified cells and progeny may be part of a multi-cellular organism such as a plant or animal with ex vivo or in vivo application of composition to desired cell types. The methods herein include a therapeutic method of treatment. The therapeutic method of treatment may comprise gene or genome editing, or gene therapy.

In one embodiment, one or more vectors described herein are used to produce a non-human transgenic animal or transgenic plant. In one embodiment, the transgenic animal is a mammal, such as a mouse, rat, or rabbit. Methods for producing transgenic animals and plants are known in the art, and generally begin with a method of cell transfection, such as described herein.

### Detection Methods Such as FISH

In one aspect, the invention provides an engineered, non-naturally occurring composition comprising a catalytically inactive IsrB polypeptide nuclease described herein, and use this system in detection methods such as fluorescence in situ hybridization (FISH). A dead IsrB polypeptide nuclease which lacks the ability to produce DNA single-strand breaks may be fused with a marker, such as fluorescent protein, such as the enhanced green fluorescent protein (eEGFP) and co-expressed with small guide RNAs to target pericentric, centric and teleomeric repeats in vivo. The dead IsrB polypeptide nuclease system can be used to visualize both repetitive sequences and individual genes in the human genome. Such new applications of labelled dead IsrB polypeptide nuclease may be important in imaging cells and studying the functional nuclear architecture, especially in cases with a small nucleus volume or complex 3-D structures. (Chen B, Gilbert L A, Cimini B A, Schnitzbauer J, Zhang W, Li G W, Park J, Blackburn E H, Weissman J S, Qi L S, Huang B. 2013. Dynamic imaging of genomic loci in living human cells by an optimized CRISPR/Cas system. Cell 155 (7): 1479-91. doi: 10.1016/j.cell.2013.12.001.)

### Patient-Specific Screening Methods

A nucleic acid-targeting system that targets DNA, e.g., trinucleotide repeats can be used to screen patients or patent samples for the presence of such repeats. The repeats can be the target of the RNA of the nucleic acid-targeting system, and if there is binding thereto by the nucleic acid-targeting system, that binding can be detected, to thereby indicate that such a repeat is present. Thus, a nucleic acid-targeting system can be used to screen patients or patient samples for the presence of the repeat. The patient can then be administered suitable compound(s) to address the condition; or, can be administered a nucleic acid-targeting system to bind to and cause insertion, deletion or mutation and alleviate the condition.

### Models of Genetic and Epigenetic Conditions

A method of the invention may be used to create a plant, an animal or cell that may be used to model and/or study genetic or epigenetic conditions of interest, such as a through a model of mutations of interest or a disease model. As used herein, “disease” refers to a disease, disorder, or indication in a subject. For example, a method of the invention may be used to create an animal or cell that comprises a modification in one or more nucleic acid sequences associated with a disease, or a plant, animal or cell in which the expression of one or more nucleic acid sequences associated with a disease are altered. Such a nucleic acid sequence may encode a disease associated protein sequence or may be a disease associated control sequence. Accordingly, it is understood that in embodiments of the invention, a plant, subject, patient, organism or cell can be a non-human subject, patient, organism or cell. Thus, the invention provides a plant, animal or cell, produced by the present methods, or a progeny thereof. The progeny may be a clone of the produced plant or animal, or may result from sexual reproduction by crossing with other individuals of the same species to introgress further desirable traits into their offspring. The cell may be in vivo or ex vivo in the cases of multicellular organisms, particularly animals or plants. In the instance where the cell is in cultured, a cell line may be established if appropriate culturing conditions are met and preferably if the cell is suitably adapted for this purpose (for instance a stem cell). Bacterial cell lines produced by the invention are also envisaged. Hence, cell lines are also envisaged.

In some methods, the disease model can be used to study the effects of mutations on the animal or cell and development and/or progression of the disease using measures commonly used in the study of the disease. Alternatively, such a disease model is useful for studying the effect of a pharmaceutically active compound on the disease.

In some methods, the disease model can be used to assess the efficacy of a potential gene therapy strategy. That is, a disease-associated gene or polynucleotide can be modified such that the disease development and/or progression is inhibited or reduced. In particular, the method comprises modifying a disease-associated gene or polynucleotide such that an altered protein is produced and, as a result, the animal or cell has an altered response. Accordingly, in some methods, a genetically modified animal may be compared with an animal predisposed to development of the disease such that the effect of the gene therapy event may be assessed.

In another embodiment, this invention provides a method of developing a biologically active agent that modulates a cell signaling event associated with a disease gene. The method comprises contacting a test compound with a cell comprising one or more vectors that drive expression of one or more of a IsrB polypeptide nuclease, and a conserved nucleotide sequence linked to a ωRNA sequence; and detecting a change in a readout that is indicative of a reduction or an augmentation of a cell signaling event associated with, e.g., a mutation in a disease gene contained in the cell.

A cell model or animal model can be constructed in combination with the method of the invention for screening a cellular function change. Such a model may be used to study the effects of a genome sequence modified by the complex of the invention on a cellular function of interest. For example, a cellular function model may be used to study the effect of a modified genome sequence on intracellular signaling or extracellular signaling. Alternatively, a cellular function model may be used to study the effects of a modified genome sequence on sensory perception. In some such models, one or more genome sequences associated with a signaling biochemical pathway in the model are modified.

Several disease models have been specifically investigated. These include de novo autism risk genes CHD8, KATNAL2, and SCN2A; and the syndromic autism (Angelman Syndrome) gene UBE3A. These genes and resulting autism models are of course preferred, but serve to show the broad applicability of the invention across genes and corresponding models. An altered expression of one or more genome sequences associated with a signaling biochemical pathway can be determined by assaying for a difference in the mRNA levels of the corresponding genes between the test model cell and a control cell, when they are contacted with a candidate agent. Alternatively, the differential expression of the sequences associated with a signaling biochemical pathway is determined by detecting a difference in the level of the encoded polypeptide or gene product.

To assay for an agent-induced alteration in the level of mRNA transcripts or corresponding polynucleotides, nucleic acid contained in a sample is first extracted according to standard methods in the art. For instance, mRNA can be isolated using various lytic enzymes or chemical solutions according to the procedures set forth in Sambrook et al. (1989), or extracted by nucleic-acid-binding resins following the accompanying instructions provided by the manufacturers. The mRNA contained in the extracted nucleic acid sample is then detected by amplification procedures or conventional hybridization assays (e.g. Northern blot analysis) according to methods widely known in the art or based on the methods exemplified herein.

For purpose of this invention, amplification means any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. In particular, the isolated RNA can be subjected to a reverse transcription assay that is coupled with a quantitative polymerase chain reaction (RT-PCR) in order to quantify the expression level of a sequence associated with a signaling biochemical pathway.

Detection of the gene expression level can be conducted in real time in an amplification assay. In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dye suitable for this application include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

In another aspect, other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are well established in the art and are taught in U.S. Pat. No. 5,210,015.

In yet another aspect, conventional hybridization assays using hybridization probes that share sequence homology with sequences associated with a signaling biochemical pathway can be performed. Typically, probes are allowed to form stable complexes with the sequences associated with a signaling biochemical pathway contained within the biological sample derived from the test subject in a hybridization reaction. It will be appreciated by one of skill in the art that where antisense is used as the probe nucleic acid, the target polynucleotides provided in the sample are chosen to be complementary to sequences of the antisense nucleic acids. Conversely, where the nucleotide probe is a sense nucleic acid, the target polynucleotide is selected to be complementary to sequences of the sense nucleic acid.

Hybridization can be performed under conditions of various stringency. Suitable hybridization conditions for the practice of the present invention are such that the recognition interaction between the probe and sequences associated with a signaling biochemical pathway is both sufficiently specific and sufficiently stable. Conditions that increase the stringency of a hybridization reaction are widely known and published in the art. See, for example, (Sambrook, et al., (1989); Nonradioactive In Situ Hybridization Application Manual, Boehringer Mannheim, second edition). The hybridization assay can be formed using probes immobilized on any solid support, including but are not limited to nitrocellulose, glass, silicon, and a variety of gene arrays. A preferred hybridization assay is conducted on high-density gene chips as described in U.S. Pat. No. 5,445,934.

For a convenient detection of the probe-target complexes formed during the hybridization assay, the nucleotide probes are conjugated to a detectable label. Detectable labels suitable for use in the present invention include any composition detectable by photochemical, biochemical, spectroscopic, immunochemical, electrical, optical or chemical means. A wide variety of appropriate detectable labels are known in the art, which include fluorescent or chemiluminescent labels, radioactive isotope labels, enzymatic or other ligands. In preferred embodiments, one will likely desire to employ a fluorescent label or an enzyme tag, such as digoxigenin, β-galactosidase, urease, alkaline phosphatase or peroxidase, avidin/biotin complex.

The detection methods used to detect or quantify the hybridization intensity will typically depend upon the label selected above. For example, radiolabels may be detected using photographic film or a phosphoimager. Fluorescent markers may be detected and quantified using a photodetector to detect emitted light. Enzymatic labels are typically detected by providing the enzyme with a substrate and measuring the reaction product produced by the action of the enzyme on the substrate; and finally colorimetric labels are detected by simply visualizing the colored label.

An agent-induced change in expression of sequences associated with a signaling biochemical pathway can also be determined by examining the corresponding gene products. Determining the protein level typically involves a) contacting the protein contained in a biological sample with an agent that specifically bind to a protein associated with a signaling biochemical pathway; and (b) identifying any agent: protein complex so formed. In one aspect of this embodiment, the agent that specifically binds a protein associated with a signaling biochemical pathway is an antibody, preferably a monoclonal antibody.

The reaction is performed by contacting the agent with a sample of the proteins associated with a signaling biochemical pathway derived from the test samples under conditions that will allow a complex to form between the agent and the proteins associated with a signaling biochemical pathway. The formation of the complex can be detected directly or indirectly according to standard procedures in the art. In the direct detection method, the agents are supplied with a detectable label and unreacted agents may be removed from the complex; the amount of remaining label thereby indicating the amount of complex formed. For such method, it is preferable to select labels that remain attached to the agents even during stringent washing conditions. It is preferable that the label does not interfere with the binding reaction. In the alternative, an indirect detection procedure may use an agent that contains a label introduced either chemically or enzymatically. A desirable label generally does not interfere with binding or the stability of the resulting agent: polypeptide complex. However, the label is typically designed to be accessible to an antibody for an effective binding and, hence generating a detectable signal.

A wide variety of labels suitable for detecting protein levels are known in the art. Non-limiting examples include radioisotopes, enzymes, colloidal metals, fluorescent compounds, bioluminescent compounds, and chemiluminescent compounds.

The amount of agent: polypeptide complexes formed during the binding reaction can be quantified by standard quantitative assays. As illustrated above, the formation of agent: polypeptide complex can be measured directly by the amount of label remained at the site of binding. In an alternative, the protein associated with a signaling biochemical pathway is tested for its ability to compete with a labeled analog for binding sites on the specific agent. In this competitive assay, the amount of label captured is inversely proportional to the amount of protein sequences associated with a signaling biochemical pathway present in a test sample.

A number of techniques for protein analysis based on the general principles outlined above are available in the art. They include but are not limited to radioimmunoassay, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

Antibodies that specifically recognize or bind to proteins associated with a signaling biochemical pathway are preferable for conducting the aforementioned protein analyses. Where desired, antibodies that recognize a specific type of post-translational modifications (e.g., signaling biochemical pathway inducible modifications) can be used. Post-translational modifications include but are not limited to glycosylation, lipidation, acetylation, and phosphorylation. These antibodies may be purchased from commercial vendors. For example, anti-phosphotyrosine antibodies that specifically recognize tyrosine-phosphorylated proteins are available from a number of vendors including Invitrogen and Perkin Elmer. Anti-phosphotyrosine antibodies are particularly useful in detecting proteins that are differentially phosphorylated on their tyrosine residues in response to an ER stress. Such proteins include but are not limited to eukaryotic translation initiation factor 2 alpha (eIF-2a). Alternatively, these antibodies can be generated using conventional polyclonal or monoclonal antibody technologies by immunizing a host animal or an antibody-producing cell with a target protein that exhibits the desired post-translational modification.

### Genome Wide Knock-Out Screening

The IsrB polypeptide nuclease and systems described herein can be used to perform efficient and cost effective functional genomic screens. Such screens can utilize IsrB polypeptide nuclease-based genome wide libraries. Such screens and libraries can provide for determining the function of genes, cellular pathways genes are involved in, and how any alteration in gene expression can result in a particular biological process. An advantage of the present invention is that the composition avoids off-target binding and its resulting side effects. This is achieved using systems arranged to have a high degree of sequence specificity for the target DNA. In preferred embodiments of the invention, the IsrB polypeptide nuclease complexes are IsrB polypeptide nuclease complexes.

In embodiments of the invention, a genome wide library may comprise a plurality of IsrB polypeptide nuclease @ RNAs, as described herein, comprising guide sequences that are capable of targeting a plurality of target sequences in a plurality of genomic loci in a population of eukaryotic cells. The population of cells may be a population of embryonic stem (ES) cells. The target sequence in the genomic locus may be a non-coding sequence. The non-coding sequence may be an intron, regulatory sequence, splice site, 3′ UTR, 5′ UTR, or polyadenylation signal. Gene function of one or more gene products may be altered by said targeting. The targeting may result in a knockout of gene function. The targeting of a gene product may comprise more than one ωRNA. A gene product may be targeted by 2, 3, 4, 5, 6, 7, 8, 9, or 10 ωRNAs, preferably 3 to 4 per gene. Off-target modifications may be minimized by exploiting the staggered double strand breaks generated by IsrB polypeptide nuclease complexes or by utilizing methods analogous to those used in composition (See, e.g., DNA targeting specificity of RNA-guided Cas nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi: 10.1038/nbt.2647 (2013)), incorporated herein by reference. The targeting may be of about 100 or more sequences. The targeting may be of about 1000 or more sequences. The targeting may be of about 20,000 or more sequences. The targeting may be of the entire genome. The targeting may be of a panel of target sequences focused on a relevant or desirable pathway. The pathway may be an immune pathway. The pathway may be a cell division pathway.

One aspect of the invention comprehends a genome wide library that may comprise a plurality of ωRNAs that may comprise guide sequences that are capable of targeting a plurality of target sequences in a plurality of genomic loci, wherein said targeting results in a knockout of gene function. This library may potentially comprise @guide RNAs that target each and every gene in the genome of an organism.

In one embodiment of the invention, the organism or subject is a eukaryote (including mammal including human) or a non-human eukaryote or a non-human animal or a non-human mammal. In one embodiment, the organism or subject is a non-human animal, and may be an arthropod, for example, an insect, or may be a nematode. In some methods of the invention the organism or subject is a plant. In some methods of the invention, the organism or subject is a mammal or a non-human mammal. A non-human mammal may be for example a rodent (preferably a mouse or a rat), an ungulate, or a primate. In some methods of the invention the organism or subject is algae, including microalgae, or is a fungus.

The knockout of gene function may comprise introducing into each cell in the population of cells a vector system of one or more vectors comprising an engineered, non-naturally occurring composition herein. The guide sequence may target a unique gene in each cell, wherein the IsrB polypeptide nuclease is operably linked to a regulatory element, wherein when transcribed, the ωRNAs comprising the @sequence directs sequence-specific binding of the IsrB polypeptide nuclease to a target sequence in the genomic loci of the unique gene, inducing cleavage of the genomic loci by the IsrB polypeptide nuclease, and confirming different knockout mutations in a plurality of unique genes in each cell of the population of cells thereby generating a gene knockout cell library. The invention comprehends that the population of cells is a population of eukaryotic cells, and in a preferred embodiment, the population of cells is a population of embryonic stem (ES) cells.

The one or more vectors may be plasmid vectors. The vector may be a single vector comprising a IsrB polypeptide nuclease, a ωRNA, and optionally, a selection marker into target cells. Not being bound by a theory, the ability to simultaneously deliver a IsrB polypeptide nuclease and hRNA through a single vector enables application to any cell type of interest, without the need to first generate cell lines that express the IsrB polypeptide nuclease. The regulatory element may be an inducible promoter. The inducible promoter may be a doxycycline inducible promoter. In some methods of the invention the expression of the guide sequence is under the control of the T7 promoter and is driven by the expression of T7 polymerase. The confirming of different knockout mutations may be by whole exome sequencing. The knockout mutation may be achieved in 100 or more unique genes. The knockout mutation may be achieved in 1000 or more unique genes. The knockout mutation may be achieved in 20,000 or more unique genes. The knockout mutation may be achieved in the entire genome. The knockout of gene function may be achieved in a plurality of unique genes which function in a particular physiological pathway or condition. The pathway or condition may be an immune pathway or condition. The pathway or condition may be a cell division pathway or condition.

Useful in the practice of the instant invention utilizing IsrB polypeptide nuclease complexes are methods used in compositions and reference is made to: Genome-Scale CRISPR-Cas Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013). [Epub ahead of print]; Published in final edited form as: Science. 2014 Jan. 3; 343 (6166): 84-87. Shalem et al. involves a new way to interrogate gene function on a genome-wide scale. Their studies showed that delivery of a genome-scale CRISPR-Cas knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hitsNF2, CUL3, TADA2B, and TADA1. The authors observed a high level of consistency between independent guide RNAs targeting the same gene and a high rate of hit confirmation, and thus demonstrated the promise of genome-scale screening with IsrB polypeptide nuclease.

Reference is also made to US patent publication number US20140357530; and PCT Patent Publication WO2014093701, hereby incorporated herein by reference. Reference is also made to NIH Press Release of Oct. 22, 2015 entitled, “Researchers identify potential alternative to CRISPR-Cas genome editing tools: New Cas enzymes shed light on evolution of CRISPR-Cas systems, which is incorporated by reference.

### Functional Alteration and Screening

In another aspect, the present invention provides for a method of functional evaluation and screening of genes. The use of the compositions to precisely deliver functional domains, to activate or repress genes or to alter epigenetic state by precisely altering the methylation site on a specific locus of interest, can be with one or more ωRNAs applied to a single cell or population of cells or with a library applied to genome in a pool of cells ex vivo or in vivo comprising the administration or expression of a library comprising a plurality of omega RNAs (comprising guide molecules) and wherein the screening further comprises use of a IsrB polypeptide nuclease, wherein the complex comprising the IsrB polypeptide nuclease is modified to comprise a heterologous functional domain. In an aspect the invention provides a method for screening a genome comprising the administration to a host or expression in a host in vivo of a library. In an aspect the invention provides a method as herein discussed further comprising an activator administered to the host or expressed in the host. In an aspect the invention provides a method as herein discussed wherein the activator is attached to a IsrB polypeptide nuclease. In an aspect the invention provides a method as herein discussed wherein the activator is attached to the N terminus or the C terminus of the IsrB polypeptide nuclease. In an aspect the invention provides a method as herein discussed wherein the activator is attached to a ωRNA loop. In an aspect the invention provides a method as herein discussed further comprising a repressor administered to the host or expressed in the host. In an aspect the invention provides a method as herein discussed, wherein the screening comprises affecting and detecting gene activation, gene inhibition, or cleavage in the locus.

It is also preferred to target endogenous (regulatory) control elements (such as enhancers and silencers) e.g. in addition to a promoter or promoter-proximal elements. Thus, the invention can also be used to target endogenous control elements (including enhancers and silencers) in addition to targeting of the promoter. These control elements can be located upstream and downstream of the transcriptional start site (TSS), starting from 200 bp from the TSS to 100 kb away. Targeting of known control elements can be used to activate or repress the gene of interest. In some cases, a single control element can influence the transcription of multiple target genes. Targeting of a single control element could therefore be used to control the transcription of multiple genes simultaneously.

Targeting of putative control elements on the other hand (e.g. by tiling the region of the putative control element as well as 200 bp up to 100 KB around the element) can be used as a means to verify such elements (by measuring the transcription of the gene of interest) or to detect novel control elements (e.g. by tiling 100 kb upstream and downstream of the TSS of the gene of interest). In addition, targeting of putative control elements can be useful in the context of understanding genetic causes of disease. Many mutations and common SNP variants associated with disease phenotypes are located outside coding regions. Targeting of such regions with either the activation or repression systems described herein can be followed by readout of transcription of either a) a set of putative targets (e.g. a set of genes located in closest proximity to the control element) or b) whole-transcriptome readout by e.g. RNAseq or microarray. This would allow for the identification of likely candidate genes involved in the disease phenotype. Such candidate genes could be useful as novel drug targets.

Histone acetyltransferase (HAT) inhibitors are mentioned herein. However, an alternative In one embodiment is for the one or more functional domains to comprise an acetyltransferase, preferably a histone acetyltransferase. These are useful in the field of epigenomics, for example in methods of interrogating the epigenome. Methods of interrogating the epigenome may include, for example, targeting epigenomic sequences. Targeting epigenomic sequences may include the guide being directed to an epigenomic target sequence. In one embodiment, include a promoter, silencer or an enhancer sequence.

### Saturating Mutagenesis

The compositions herein can be used to perform saturating or deep scanning mutagenesis of genomic loci in conjunction with a cellular phenotype—for instance, for determining critical minimal features and discrete vulnerabilities of functional elements required for gene expression, drug resistance, and reversal of disease. By saturating or deep scanning mutagenesis is meant that every or essentially every DNA base is cut within the genomic loci. A library of Cas1 effector protein ωRNAs may be introduced into a population of cells. The library may be introduced, such that each cell receives a single hRNA. In the case where the library is introduced by transduction of a viral vector, as described herein, a low multiplicity of infection (MOI) is used. The library may include ωRNAs targeting every sequence upstream of a TAM sequence in a genomic locus. The library may include at least 100 non-overlapping genomic sequences upstream of a TAM sequence for every 1000 base pairs within the genomic locus. The library may include hRNAs targeting sequences upstream of at least one different TAM sequence. The composition may include more than one IsrB polypeptide nuclease. Any IsrB polypeptide nuclease protein as described herein, including orthologues or engineered IsrB polypeptide nuclease that recognize different TAM sequences may be used. The frequency of off target sites for a ωRNA may be less than 500. Off target scores may be generated to select ωRNAs with the lowest off target sites. Any phenotype determined to be associated with cutting at a hRNA target site may be confirmed by using ωRNAs targeting the same site in a single experiment. Validation of a target site may also be performed by using a modified IsrB polypeptide nuclease, as described herein, and two hRNAs targeting the genomic site of interest. Not being bound by a theory, a target site is a true hit if the change in phenotype is observed in validation experiments.

The genomic loci may include at least one continuous genomic region. The at least one continuous genomic region may comprise up to the entire genome. The at least one continuous genomic region may comprise a functional element of the genome. The functional element may be within a non-coding region, coding gene, intronic region, promoter, or enhancer. The at least one continuous genomic region may comprise at least 1 kb, preferably at least 50 kb of genomic DNA. The at least one continuous genomic region may comprise a transcription factor binding site. The at least one continuous genomic region may comprise a region of DNase I hypersensitivity. The at least one continuous genomic region may comprise a transcription enhancer or repressor element. The at least one continuous genomic region may comprise a site enriched for an epigenetic signature. The at least one continuous genomic DNA region may comprise an epigenetic insulator. The at least one continuous genomic region may comprise two or more continuous genomic regions that physically interact. Genomic regions that interact may be determined by ‘4C technology’. 4C technology allows the screening of the entire genome in an unbiased manner for DNA segments that physically interact with a DNA fragment of choice, as is described in Zhao et al. ((2006) Nat Genet 38, 1341-7) and in U.S. Pat. No. 8,642,295, both incorporated herein by reference in its entirety. The epigenetic signature may be histone acetylation, histone methylation, histone ubiquitination, histone phosphorylation, DNA methylation, or a lack thereof.

The compositions for saturating or deep scanning mutagenesis can be used in a population of cells. The compositions can be used in eukaryotic cells, including but not limited to mammalian and plant cells. The population of cells may be prokaryotic cells. The population of eukaryotic cells may be a population of embryonic stem (ES) cells, neuronal cells, epithelial cells, immune cells, endocrine cells, muscle cells, erythrocytes, lymphocytes, plant cells, or yeast cells.

In one aspect, the present invention provides for a method of screening for functional elements associated with a change in a phenotype. The library may be introduced into a population of cells that are adapted to contain a IsrB polypeptide nuclease. The cells may be sorted into at least two groups based on the phenotype. The phenotype may be expression of a gene, cell growth, or cell viability. The relative representation of the ωRNAs present in each group are determined, whereby genomic sites associated with the change in phenotype are determined by the representation of ωRNAs present in each group. The change in phenotype may be a change in expression of a gene of interest. The gene of interest may be upregulated, downregulated, or knocked out. The cells may be sorted into a high expression group and a low expression group. The population of cells may include a reporter construct that is used to determine the phenotype. The reporter construct may include a detectable marker. Cells may be sorted by use of the detectable marker.

In another aspect, the present invention provides for a method of screening for genomic sites associated with resistance to a chemical compound. The chemical compound may be a drug or pesticide. The library may be introduced into a population of cells that are adapted to contain a IsrB polypeptide nuclease, wherein each cell of the population contains no more than one ωRNA; the population of cells are treated with the chemical compound; and the representation of ωRNA are determined after treatment with the chemical compound at a later time point as compared to an early time point, whereby genomic sites associated with resistance to the chemical compound are determined by enrichment of ωRNAs. Representation of ωRNAs may be determined by deep sequencing methods.

Useful in the practice of the instant invention utilizing compositions are methods used in compositions and reference is made to the article entitled BCL11A enhancer dissection by Cas-mediated in situ saturating mutagenesis. Canver, M. C., Smith, E. C., Sher, F., Pinello, L., Sanjana, N. E., Shalem, O., Chen, D. D., Schupp, P. G., Vinjamur, D. S., Garcia, S. P., Luc, S., Kurita, R., Nakamura, Y., Fujiwara, Y., Maeda, T., Yuan, G., Zhang, F., Orkin, S. H., & Bauer, D. E. DOI: 10.1038/nature15521, published online Sep. 16, 2015, the article is herein incorporated by reference and discussed briefly below:

Canver et al. involves novel pooled guide RNA libraries to perform in situ saturating mutagenesis of the human and mouse BCL11A erythroid enhancers previously identified as an enhancer associated with fetal hemoglobin (HbF) level and whose mouse ortholog is necessary for erythroid BCL11A expression. This approach revealed critical minimal features and discrete vulnerabilities of these enhancers. Through editing of primary human progenitors and mouse transgenesis, the authors validated the BCL11A erythroid enhancer as a target for HbF reinduction. The authors generated a detailed enhancer map that informs therapeutic genome editing.

### Modification of a Cell or Organism

The present disclosure further provides cells comprising one or more components of the systems herein, e.g., the IsrB polypeptide nuclease and/or ωRNA(s). Also provided include cells modified by the systems and methods herein, and cell cultures, tissues, organs, organism comprising such cells or progeny thereof. The invention In one embodiment comprehends a method of modifying a cell or organism. The cell may be a prokaryotic cell or a eukaryotic cell. The cell may be a mammalian cell. The mammalian cell many be a non-human primate, bovine, porcine, rodent or mouse cell. The cell may be a non-mammalian eukaryotic cell such as poultry, fish or shrimp. The cell may also be a plant cell. The plant cell may be of a crop plant such as cassava, corn, sorghum, wheat, or rice. The plant cell may also be of an algae, tree or vegetable. The modification introduced to the cell by the present invention may be such that the cell and progeny of the cell are altered for improved production of biologic products such as an antibody, starch, alcohol or other desired cellular output. The modification introduced to the cell by the present invention may be such that the cell and progeny of the cell include an alteration that changes the biologic product produced.

### Therapeutic Uses and Methods of Treatment

Also provided herein are methods of diagnosing, prognosing, treating, and/or preventing a disease, state, or condition in or of a subject. Generally, the methods of diagnosing, prognosing, treating, and/or preventing a disease, state, or condition in or of a subject can include modifying a polynucleotide in a subject or cell thereof using a composition, system, or component thereof described herein and/or include detecting a diseased or healthy polynucleotide in a subject or cell thereof using a composition, system, or component thereof described herein. In one embodiment, the method of treatment or prevention can include using a composition, system, or component thereof to modify a polynucleotide of an infectious organism (e.g. bacterial or virus) within a subject or cell thereof. In one embodiment, the method of treatment or prevention can include using a composition, system, or component thereof to modify a polynucleotide of an infectious organism or symbiotic organism within a subject. The composition, system, and components thereof can be used to develop models of diseases, states, or conditions. The composition, system, and components thereof can be used to detect a disease state or correction thereof, such as by a method of treatment or prevention described herein. The composition, system, and components thereof can be used to screen and select cells that can be used, for example, as treatments or preventions described herein. The composition, system, and components thereof can be used to develop biologically active agents that can be used to modify one or more biologic functions or activities in a subject or a cell thereof.

In general, the method can include delivering a composition, system, and/or component thereof to a subject or cell thereof, or to an infectious or symbiotic organism by a suitable delivery technique and/or composition. Once administered the components can operate as described elsewhere herein to elicit a nucleic acid modification event. In some aspects, the nucleic acid modification event can occur at the genomic, epigenomic, and/or transcriptomic level. DNA and/or RNA cleavage, gene activation, and/or gene deactivation can occur. Additional features, uses, and advantages are described in greater detail below. On the basis of this concept, several variations are appropriate to elicit a genomic locus event, including DNA cleavage, gene activation, or gene deactivation. Using the provided compositions, the person skilled in the art can advantageously and specifically target single or multiple loci with the same or different functional domains to elicit one or more genomic locus events. In addition to treating and/or preventing a disease in a subject, the compositions may be applied in a wide variety of methods for screening in libraries in cells and functional modeling in vivo (e.g. gene activation of lincRNA and identification of function; gain-of-function modeling; loss-of-function modeling; the use the compositions of the invention to establish cell lines and transgenic animals for optimization and screening purposes).

The composition, system, and components thereof described elsewhere herein can be used to treat and/or prevent a disease, such as a genetic and/or epigenetic disease, in a subject. The composition, system, and components thereof described elsewhere herein can be used to treat and/or prevent genetic infectious diseases in a subject, such as bacterial infections, viral infections, fungal infections, parasite infections, and combinations thereof. The composition, system, and components thereof described elsewhere herein can be used to modify the composition or profile of a microbiome in a subject, which can in turn modify the health status of the subject. The composition, system, described herein can be used to modify cells ex vivo, which can then be administered to the subject whereby the modified cells can treat or prevent a disease or symptom thereof. This is also referred to in some contexts as adoptive therapy. The composition, system, described herein can be used to treat mitochondrial diseases, where the mitochondrial disease etiology involves a mutation in the mitochondrial DNA.

Also provided is a method of treating a subject, e.g., a subject in need thereof, comprising inducing gene editing by transforming the subject with the polynucleotide encoding one or more components of the composition, system, or complex or any of polynucleotides or vectors described herein and administering them to the subject. A suitable repair template may also be provided, for example delivered by a vector comprising said repair template. The repair template may be a recombination template herein. Also provided is a method of treating a subject, e.g., a subject in need thereof, comprising inducing transcriptional activation or repression of multiple target gene loci by transforming the subject with the polynucleotides or vectors described herein, wherein said polynucleotide or vector encodes or comprises one or more components of composition, system, complex or component thereof comprising multiple IscB polypeptide nucleases. Where any treatment is occurring ex vivo, for example in a cell culture, then it will be appreciated that the term ‘subject’ may be replaced by the phrase “cell or cell culture.”

Also provided is a method of treating a subject, e.g., a subject in need thereof, comprising inducing gene editing by transforming the subject with the IsrB polypeptide nuclease(s), advantageously encoding and expressing in vivo the remaining portions of the composition, system, (e.g., ωRNA). A suitable repair template may also be provided, for example delivered by a vector comprising said repair template. Also provided is a method of treating a subject, e.g., a subject in need thereof, comprising inducing transcriptional activation or repression by transforming the subject with the IsrB polypeptide nuclease(s) advantageously encoding and expressing in vivo the remaining portions of the composition, system, (e.g., ωRNA,); advantageously In one embodiment the IsrB polypeptide nuclease is a catalytically inactive IsrB polypeptide nuclease and includes one or more associated functional domains. Where any treatment is occurring ex vivo, for example in a cell culture, then it will be appreciated that the term ‘subject’ may be replaced by the phrase “cell or cell culture.”

One or more components of the composition and system described herein can be included in a composition, such as a pharmaceutical composition, and administered to a host individually or collectively. Alternatively, these components may be provided in a single composition for administration to a host. Administration to a host may be performed via viral vectors known to the skilled person or described herein for delivery to a host (e.g. lentiviral vector, adenoviral vector, AAV vector). As explained herein, use of different selection markers (e.g. for lentiviral hRNA selection) and concentration of ωRNA (e.g. dependent on whether multiple ωRNAs are used) may be advantageous for eliciting an improved effect.

Thus, also described herein are methods of inducing one or more polynucleotide modifications in a eukaryotic or prokaryotic cell or component thereof (e.g., a mitochondria) of a subject, infectious organism, and/or organism of the microbiome of the subject. The modification can include the introduction, deletion, or substitution of one or more nucleotides at a target sequence of a polynucleotide of one or more cell(s). The modification can occur in vitro, ex vivo, in situ, or in vivo.

In one embodiment, the method of treating or inhibiting a condition or a disease caused by one or more mutations in a genomic locus in a eukaryotic organism or a non-human organism can include manipulation of a target sequence within a coding, non-coding or regulatory element of said genomic locus in a target sequence in a subject or a non-human subject in need thereof comprising modifying the subject or a non-human subject by manipulation of the target sequence and wherein the condition or disease is susceptible to treatment or inhibition by manipulation of the target sequence including providing treatment comprising delivering a composition comprising the particle delivery system or the delivery system or the virus particle of any one of the above embodiment or the cell of any one of the above embodiment.

Also provided herein is the use of the particle delivery system or the delivery system or the virus particle of any one of the above embodiments or the cell of any one of the above embodiment in ex vivo or in vivo gene or genome editing; or for use in in vitro, ex vivo or in vivo gene therapy. Also provided herein are particle delivery systems, non-viral delivery systems, and/or the virus particle of any one of the above embodiments or the cell of any one of the above embodiments used in the manufacture of a medicament for in vitro, ex vivo or in vivo gene or genome editing or for use in in vitro, ex vivo or in vivo gene therapy or for use in a method of modifying an organism or a non-human organism by manipulation of a target sequence in a genomic locus associated with a disease or in a method of treating or inhibiting a condition or disease caused by one or more mutations in a genomic locus in a eukaryotic organism or a non-human organism.

In one embodiment, polynucleotide modification can include the introduction, deletion, or substitution of 1-75 nucleotides at each target sequence of said polynucleotide of said cell(s). The modification can include the introduction, deletion, or substitution of at least 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence. The modification can include the introduction, deletion, or substitution of at least 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The modification can include the introduction, deletion, or substitution of at least 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The modification can include the introduction, deletion, or substitution of at least 20, 21, 22, 23, 24, 25, 26, 27, 28, 29, 30, 35, 40, 45, 50, or 75 nucleotides at each target sequence of said cell(s). The modification can include the introduction, deletion, or substitution of at least 40, 45, 50, 75, 100, 200, 300, 400 or 500 nucleotides at each target sequence of said cell(s). The modification can include the introduction, deletion, or substitution of at least 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, or 9900 to 10000 nucleotides at each target sequence of said cell(s).

In one embodiment, the modifications can include the introduction, deletion, or substitution of nucleotides at each target sequence of said cell(s) via nucleic acid components (e.g., ωRNA(s)), such as those mediated by a composition, system, or a component thereof described elsewhere herein. In one embodiment, the modifications can include the introduction, deletion, or substitution of nucleotides at a target or random sequence of said cell(s) via a composition, system, or technique.

In one embodiment, the composition, system, or component thereof can promote Non-Homologous End-Joining (NHEJ). In one embodiment, modification of a polynucleotide by a composition, system, or a component thereof, such as a diseased polynucleotide, can include NHEJ. In one embodiment, promotion of this repair pathway by the composition, system, or a component thereof can be used to target gene or polynucleotide specific knock-outs and/or knock-ins. In one embodiment, promotion of this repair pathway by the composition, system, or a component thereof can be used to generate NHEJ-mediated indels. Nuclease-induced NHEJ can also be used to remove (e.g., delete) sequence in a gene of interest. Generally, NHEJ repairs a double-strand break in the DNA by joining together the two ends; however, generally, the original sequence is restored only if two compatible ends, exactly as they were formed by the double-strand break, are perfectly ligated. The DNA ends of the single-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. The indel can range in size from 1-50 or more base pairs. In one embodiment thee indel can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 base pairs or more. If a double-strand break is targeted near to a short target sequence, the deletion mutations caused by the NHEJ repair often span, and therefore remove, the unwanted nucleotides. For the deletion of larger DNA segments, introducing two single-strand breaks, one on each side of the sequence, can result in NHEJ between the ends with removal of the entire intervening sequence. Both of these approaches can be used to delete specific DNA sequences.

In one embodiment, composition, system, mediated NHEJ can be used in the method to delete small sequence motifs. In one embodiment, composition, system, mediated NHEJ can be used in the method to generate NHEJ-mediate indels that can be targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp). In an embodiment, in which a ωRNA and IsrB polypeptide nuclease generates a single-strand break for the purpose of inducing NHEJ-mediated indels, a ωRNA may be configured to position one single-strand break in close proximity to a nucleotide of the target position. In an embodiment, the cleavage site may be between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position). In an embodiment, in which two ωRNA complexing with one or more nickases induce two single strand breaks for the purpose of inducing NHEJ-mediated indels, two @ RNAs may be configured to position two single-strand breaks to provide for NHEJ repair a nucleotide of the target position.

For minimization of toxicity and off-target effect, it may be important to control the concentration of IsrB polypeptide nuclease mRNA and ωRNA delivered. Optimal concentrations of IsrB polypeptide nuclease mRNA and ωRNA can be determined by testing different concentrations in a cellular or non-human eukaryote animal model and using deep sequencing the analyze the extent of modification at potential off-target genomic loci. Alternatively, to minimize the level of toxicity and off-target effect, nickase mRNA (for example S. pyogenes Cas9 with the D10A mutation) can be delivered with a pair of guide RNAs targeting a site of interest. Guide sequences and strategies to minimize toxicity and off-target effects can be as in International Patent Publication No. WO 2014/093622 (PCT/US2013/074667); or, via mutation. Others are as described elsewhere herein.

Typically, in the context of an endogenous IsrB polypeptide nuclease, formation of a IsrB polypeptide nuclease or complex (comprising a ωRNA sequence hybridized to a target sequence and complexed with one or more IsrB polypeptide nucleases) results in cleavage, nicking, and/or another modification of one or both strands in or near (e.g. within 1, 2, 3, 4, 5, 6, 7, 8, 9, 10, 20, 50, or more base pairs from) the target sequence.

In one embodiment, a method of modifying a target polynucleotide in a cell to treat or prevent a disease can include allowing a composition, system, or component thereof to bind to the target polynucleotide, e.g., to effect cleavage, nicking, or other modification as the composition, system, is capable of said target polynucleotide, thereby modifying the target polynucleotide, wherein the composition, system, or component thereof, complex with a guide sequence, and hybridize said guide sequence to a target sequence within the target polynucleotide, wherein said guide sequence is optionally linked to a @ RNA scaffold sequence. In some of these embodiments, the composition, system, or component thereof can be or include a IsrB polypeptide nuclease complexed with a guide sequence. In one embodiment, modification can include cleaving or nicking one or two strands at the location of the target sequence by one or more components of the composition, system, or component thereof.

The cleavage, nicking, or other modification capable of being performed by the composition, system, can modify transcription of a target polynucleotide. In one embodiment, modification of transcription can include decreasing transcription of a target polynucleotide. In one embodiment, modification can include increasing transcription of a target polynucleotide. In one embodiment, the method includes repairing said cleaved target polynucleotide by homologous recombination with a recombination template polynucleotide, wherein said repair results in a modification such as, but not limited to, an insertion, deletion, or substitution of one or more nucleotides of said target polynucleotide. In one embodiment, said modification results in one or more amino acid changes in a protein expressed from a gene comprising the target sequence. In one embodiment, the modification imparted by the composition, system, or component thereof provides a transcript and/or protein that can correct a disease or a symptom thereof, including but not limited to, any of those described in greater detail elsewhere herein.

In one embodiment, the method of treating or preventing a disease can include delivering one or more vectors or vector systems to a cell, such as a eukaryotic or prokaryotic cell, wherein one or more vectors or vector systems include the composition, system, or component thereof. In one embodiment, the vector(s) or vector system(s) can be a viral vector or vector system, such as an AAV or lentiviral vector system, which are described in greater detail elsewhere herein. In one embodiment, the method of treating or preventing a disease can include delivering one or more viral particles, such as an AAV or lentiviral particle, containing the composition, system, or component thereof. In one embodiment, the viral particle has a tissue specific tropism. In one embodiment, the viral particle has a liver, muscle, eye, heart, pancreas, kidney, neuron, epithelial cell, endothelial cell, astrocyte, glial cell, immune cell, or red blood cell specific tropism.

It will be understood that the composition and system, according to the invention as described herein, such as the composition and system, for use in the methods according to the invention as described herein, may be suitably used for any type of application known for composition, system, preferably in eukaryotes. In certain aspects, the application is therapeutic, preferably therapeutic in a eukaryote organism, such as including but not limited to animals (including human), plants, algae, fungi (including yeasts), etc. Alternatively, or in addition, in certain aspects, the application may involve accomplishing or inducing one or more particular traits or characteristics, such as genotypic and/or phenotypic traits or characteristics, as also described elsewhere herein.

### Treating Diseases of the Circulatory System

In one embodiment, the composition, system, and/or component thereof described herein can be used to treat and/or prevent a circulatory system disease. Exemplary disease is provided, for example, in Table 6. In one embodiment the plasma exosomes of Wahlgren et al. (Nucleic Acids Research, 2012, Vol. 40, No. 17 e130) can be used to deliver the composition, system, and/or component thereof described herein to the blood. In one embodiment, the circulatory system disease can be treated by using a lentivirus to deliver the composition, system, described herein to modify hematopoietic stem cells (HSCs) in vivo or ex vivo (see e.g. Drakopoulou, “Review Article, The Ongoing Challenge of Hematopoietic Stem Cell-Based Gene Therapy for β-Thalassemia,” Stem Cells International, Volume 2011, Article ID 987980, 10 pages, doi: 10.4061/2011/987980, which can be adapted for use with the composition, system, herein in view of the description herein). In one embodiment, the circulatory system disorder can be treated by correcting HSCs as to the disease using a composition, system, herein or a component thereof, wherein the composition, system, optionally includes a suitable HDR repair template (see e.g. Cavazzana, “Outcomes of Gene Therapy for β-Thalassemia Major via Transplantation of Autologous Hematopoietic Stem Cells Transduced Ex Vivo with a Lentiviral βA-T87Q-Globin Vector.”; Cavazzana-Calvo, “Transfusion independence and HMGA2 activation after gene therapy of human β-thalassaemia”, Nature 467, 318-322 (16 Sep. 2010) doi: 10.1038/nature09328; Nienhuis, “Development of Gene Therapy for Thalassemia, Cold Spring Harbor Perspectives in Medicine, doi: 10.1101/cshperspect.a011833 (2012), LentiGlobin BB305, a lentiviral vector containing an engineered β-globin gene (BA-T87Q); and Xie et al., “Seamless gene correction of β-thalassaemia mutations in patient-specific iPSCs using CRISPR/Cas9 and piggyback” Genome Research gr. 173427.114 (2014) www.genome.org/cgi/doi/10.1101/gr.173427.114 (Cold Spring Harbor Laboratory Press; [1599] Watts, “Hematopoietic Stem Cell Expansion and Gene Therapy” Cytotherapy 13 (10): 1164-1171. doi: 10.3109/14653249.2011.620748 (2011), which can be adapted for use with the composition, system, herein in view of the description herein). In one embodiment, iPSCs can be modified using a composition, system, described herein to correct a disease polynucleotide associated with a circulatory disease. In this regard, the teachings of Xu et al. (Sci Rep. 2015 Jul. 9; 5:12065. doi: 10.1038/srep12065) and Song et al. (Stem Cells Dev. 2015 May 1; 24 (9): 1053-65. doi: 10.1089/scd.2014.0347. Epub 2015 Feb. 5) with respect to modifying iPSCs can be adapted for use in view of the description herein with the composition, system, described herein.

The term “Hematopoietic Stem Cell” or “HSC” refers broadly those cells considered to be an HSC, e.g., blood cells that give rise to all the other blood cells and are derived from mesoderm; located in the red bone marrow, which is contained in the core of most bones. HSCs of the invention include cells having a phenotype of hematopoietic stem cells, identified by small size, lack of lineage (lin) markers, and markers that belong to the cluster of differentiation series, like: CD34, CD38, CD90, CD133, CD105, CD45, and also c-kit,-the receptor for stem cell factor. Hematopoietic stem cells are negative for the markers that are used for detection of lineage commitment, and are, thus, called Lin−; and, during their purification by FACS, a number of up to 14 different mature blood-lineage markers, e.g., CD13 & CD33 for myeloid, CD71 for erythroid, CD19 for B cells, CD61 for megakaryocytic, etc. for humans; and, B220 (murine CD45) for B cells, Mac-1 (CD11b/CD18) for monocytes, Gr-1 for Granulocytes, Ter119 for erythroid cells, I17Ra, CD3, CD4, CD5, CD8 for T cells, etc. Mouse HSC markers: CD34lo/−, SCA-1+, Thy1.1+/lo, CD38+, C-kit+, lin−, and Human HSC markers: CD34+, CD59+, Thy1/CD90+, CD38lo/−, C-kit/CD117+, and lin−. HSCs are identified by markers. Hence in embodiments discussed herein, the HSCs can be CD34+ cells. HSCs can also be hematopoietic stem cells that are CD34−/CD38−. Stem cells that may lack c-kit on the cell surface that are considered in the art as HSCs are within the ambit of the invention, as well as CD133+ cells likewise considered HSCs in the art.

In one embodiment, the treatment or prevention for treating a circulatory system or blood disease can include modifying a human cord blood cell with any modification described herein. In one embodiment, the treatment or prevention for treating a circulatory system or blood disease can include modifying a granulocyte colony-stimulating factor-mobilized peripheral blood cell (mPB) with any modification described herein. In one embodiment, the human cord blood cell or mPB can be CD34+. In one embodiment, the cord blood cell(s) or mPB cell(s) modified can be autologous. In one embodiment, the cord blood cell(s) or mPB cell(s) can be allogenic. In addition to the modification of the disease gene(s), allogenic cells can be further modified using the composition, system, described herein to reduce the immunogenicity of the cells when delivered to the recipient. Such techniques are described elsewhere herein and e.g. Cartier, “MINI-SYMPOSIUM: X-Linked Adrenoleukodystrophypa, Hematopoietic Stem Cell Transplantation and Hematopoietic Stem Cell Gene Therapy in X-Linked Adrenoleukodystrophy,” Brain Pathology 20 (2010) 857-862, which can be adapted for use with the composition, system, herein. The modified cord blood cell(s) or mPB cell(s) can be optionally expanded in vitro. The modified cord blood cell(s) or mPB cell(s) can be derived to a subject in need thereof using any suitable delivery technique.

The compositions may be engineered to target genetic locus or loci in HSCs. In one embodiment, the IsrB polypeptide nuclease(s) can be codon-optimized for a eukaryotic cell and especially a mammalian cell, e.g., a human cell, for instance, HSC, or iPSC and hRNA targeting a locus or loci in HSC, such as circulatory disease, can be prepared. These may be delivered via particles. The particles may be formed by the IsrB polypeptide nuclease and the ωRNA being admixed. The @ RNA and IsrB polypeptide nuclease mixture can be, for example, admixed with a mixture comprising or consisting essentially of or consisting of surfactant, phospholipid, biodegradable polymer, lipoprotein and alcohol, whereby particles containing the ωRNA and IsrB polypeptide nuclease may be formed. The invention comprehends so making particles and particles from such a method as well as uses thereof. Particles suitable delivery of the composition in the context of blood or circulatory system or HSC delivery to the blood or circulatory system are described in greater detail elsewhere herein.

In one embodiment, after ex vivo modification the HSCs or iPCS can be expanded prior to administration to the subject. Expansion of HSCs can be via any suitable method such as that described by, Lee, “Improved ex vivo expansion of adult hematopoietic stem cells by overcoming CUL4-mediated degradation of HOXB4.” Blood. 2013 May 16; 121 (20): 4082-9. doi: 10.1182/blood-2012-09-455204. Epub 2013 Mar. 21.

In one embodiment, the HSCs or iPSCs modified can be autologous. In one embodiment, the HSCs or iPSCs can be allogenic. In addition to the modification of the disease gene(s), allogenic cells can be further modified using the composition, system, described herein to reduce the immunogenicity of the cells when delivered to the recipient. Such techniques are described elsewhere herein and e.g. Cartier, “MINI-SYMPOSIUM: X-Linked Adrenoleukodystrophypa, Hematopoietic Stem Cell Transplantation and Hematopoietic Stem Cell Gene Therapy in X-Linked Adrenoleukodystrophy,” Brain Pathology 20 (2010) 857-862, which can be adapted for use with the composition, system, herein.

### Treating Neurological Diseases

In one embodiment, the compositions, systems, described herein can be used to treat diseases of the brain and CNS. Delivery options for the brain include encapsulation of IsrB polypeptide nuclease and ωRNA in the form of either DNA or RNA into liposomes and conjugating to molecular Trojan horses for trans-blood brain barrier (BBB) delivery. Molecular Trojan horses have been shown to be effective for delivery of B-gal expression vectors into the brain of non-human primates. The same approach can be used to delivery vectors containing IsrB polypeptide nuclease and ωRNA. For instance, Xia C F and Boado R J, Pardridge W M (“Antibody-mediated targeting of siRNA via the human insulin receptor using avidin-biotin technology.” Mol Pharm. 2009 May-Jun; 6 (3): 747-51. doi: 10.1021/mp800194) describes how delivery of short interfering RNA (siRNA) to cells in culture, and in vivo, is possible with combined use of a receptor-specific monoclonal antibody (mAb) and avidin-biotin technology. The authors also report that because the bond between the targeting mAb and the siRNA is stable with avidin-biotin technology, and RNAi effects at distant sites such as brain are observed in vivo following an intravenous administration of the targeted siRNA, the teachings of which can be adapted for use with the compositions, systems, herein. In other embodiments, an artificial virus can be generated for CNS and/or brain delivery. See e.g. Zhang et al. (Mol Ther. 2003 January; 7 (1): 11-8.)), the teachings of which can be adapted for use with the compositions, systems, herein.

### Treating Hearing Diseases

In one embodiment the composition and system described herein can be used to treat a hearing disease or hearing loss in one or both ears. Deafness is often caused by lost or damaged hair cells that cannot relay signals to auditory neurons. In such cases, cochlear implants may be used to respond to sound and transmit electrical signals to the nerve cells. But these neurons often degenerate and retract from the cochlea as fewer growth factors are released by impaired hair cells.

In one embodiment, the composition, system, or modified cells can be delivered to one or both ears for treating or preventing hearing disease or loss by any suitable method or technique. Suitable methods and techniques include, but are not limited to, those set forth in US Patent Publication No. 20120328580 describes injection of a pharmaceutical composition into the ear (e.g., auricular administration), such as into the luminae of the cochlea (e.g., the Scala media, Sc vestibulae, and Sc tympani), e.g., using a syringe, e.g., a single-dose syringe. For example, one or more of the compounds described herein can be administered by intratympanic injection (e.g., into the middle ear), and/or injections into the outer, middle, and/or inner ear; administration in situ, via a catheter or pump (see e.g. Mckenna et al., (U.S. Patent Publication No. 2006/0030837) and Jacobsen et al., (U.S. Pat. No. 7,206,639); administration in combination with a mechanical device such as a cochlear implant or a hearing aid, which is worn in the outer ear (see e.g. U.S. Patent Publication No. 2007/0093878, which provides an exemplary cochlear implant suitable for delivery of the compositions, systems, described herein to the ear). Such methods are routinely used in the art, for example, for the administration of steroids and antibiotics into human ears. Injection can be, for example, through the round window of the ear or through the cochlear capsule. Other inner ear administration methods are known in the art (see, e.g., Salt and Plontke, Drug Discovery Today, 10:1299-1306, 2005). In one embodiment, a catheter or pump can be positioned, e.g., in the ear (e.g., the outer, middle, and/or inner ear) of a patient during a surgical procedure. In one embodiment, a catheter or pump can be positioned, e.g., in the ear (e.g., the outer, middle, and/or inner ear) of a patient without the need for a surgical procedure.

In general, the cell therapy methods described in US Patent Publication No. 20120328580 can be used to promote complete or partial differentiation of a cell to or towards a mature cell type of the inner ear (e.g., a hair cell) in vitro. Cells resulting from such methods can then be transplanted or implanted into a patient in need of such treatment. The cell culture methods required to practice these methods, including methods for identifying and selecting suitable cell types, methods for promoting complete or partial differentiation of selected cells, methods for identifying complete or partially differentiated cell types, and methods for implanting complete or partially differentiated cells are described below.

Cells suitable for use in the present invention include, but are not limited to, cells that are capable of differentiating completely or partially into a mature cell of the inner ear, e.g., a hair cell (e.g., an inner and/or outer hair cell), when contacted, e.g., in vitro, with one or more of the compounds described herein. Exemplary cells that are capable of differentiating into a hair cell include, but are not limited to stem cells (e.g., inner ear stem cells, adult stem cells, bone marrow derived stem cells, embryonic stem cells, mesenchymal stem cells, skin stem cells, iPS cells, and fat derived stem cells), progenitor cells (e.g., inner ear progenitor cells), support cells (e.g., Deiters' cells, pillar cells, inner phalangeal cells, tectal cells and Hensen's cells), and/or germ cells. The use of stem cells for the replacement of inner ear sensory cells is described in Li et al., (U.S. Patent Publication No. 2005/0287127) and Li et al., (U.S. patent application Ser. No. 11/953,797). The use of bone marrow derived stem cells for the replacement of inner ear sensory cells is described in Edge et al., PCT/US2007/084654. iPS cells are described, e.g., at Takahashi et al., Cell, Volume 131, Issue 5, Pages 861-872 (2007); Takahashi and Yamanaka, Cell 126, 663-76 (2006); Okita et al., Nature 448, 260-262 (2007); Yu, J. et al., Science 318 (5858): 1917-1920 (2007); Nakagawa et al., Nat. Biotechnol. 26:101-106 (2008); and Zaehres and Scholer, Cell 131 (5): 834-835 (2007). Such suitable cells can be identified by analyzing (e.g., qualitatively or quantitatively) the presence of one or more tissue specific genes. For example, gene expression can be detected by detecting the protein product of one or more tissue-specific genes. Protein detection techniques involve staining proteins (e.g., using cell extracts or whole cells) using antibodies against the appropriate antigen. In this case, the appropriate antigen is the protein product of the tissue-specific gene expression. Although, in principle, a first antibody (i.e., the antibody that binds the antigen) can be labeled, it is more common (and improves the visualization) to use a second antibody directed against the first (e.g., an anti-IgG). This second antibody is conjugated either with fluorochromes, or appropriate enzymes for colorimetric reactions, or gold beads (for electron microscopy), or with the biotin-avidin system, so that the location of the primary antibody, and thus the antigen, can be recognized.

The composition and system may be delivered to the ear by direct application of pharmaceutical composition to the outer ear, with compositions modified from US Patent Publication No. 20110142917. In one embodiment the pharmaceutical composition is applied to the ear canal. Delivery to the ear may also be referred to as aural or otic delivery.

In one embodiment, the compositions, systems, or components thereof and/or vectors or vector systems can be delivered to ear via a transfection to the inner ear through the intact round window by a novel proteidic delivery technology which may be applied to the nucleic acid-targeting system of the present invention (see, e.g., Qi et al., Gene Therapy (2013), 1-9). About 40 μl of 10 mM RNA may be contemplated as the dosage for administration to the ear.

According to Rejali et al. (Hear Res. 2007 June; 228 (1-2): 180-7), cochlear implant function can be improved by good preservation of the spiral ganglion neurons, which are the target of electrical stimulation by the implant and brain derived neurotrophic factor (BDNF) has previously been shown to enhance spiral ganglion survival in experimentally deafened ears. Rejali et al. tested a modified design of the cochlear implant electrode that includes a coating of fibroblast cells transduced by a viral vector with a BDNF gene insert. To accomplish this type of ex vivo gene transfer, Rejali et al. transduced guinea pig fibroblasts with an adenovirus with a BDNF gene cassette insert, and determined that these cells secreted BDNF and then attached BDNF-secreting cells to the cochlear implant electrode via an agarose gel, and implanted the electrode in the scala tympani. Rejali et al. determined that the BDNF expressing electrodes were able to preserve significantly more spiral ganglion neurons in the basal turns of the cochlea after 48 days of implantation when compared to control electrodes and demonstrated the feasibility of combining cochlear implant therapy with ex vivo gene transfer for enhancing spiral ganglion neuron survival. Such a system may be applied to the nucleic acid-targeting system of the present invention for delivery to the ear.

In one embodiment, the system set forth in Mukherjea et al. (Antioxidants & Redox Signaling, Volume 13, Number 5, 2010) can be adapted for transtympanic administration of the composition, system, or component thereof to the ear. In one embodiment, a dosage of about 2 mg to about 4 mg of IscB polypeptide or CRISPR-associated IscB polypeptide nuclease for administration to a human.

In one embodiment, the system set forth in [Jung et al. (Molecular Therapy, vol. 21 no. 4, 834-841 Apr. 2013) can be adapted for vestibular epithelial delivery of the composition, system, or component thereof to the ear. In one embodiment, a dosage of about 1 to about 30 mg of IscB polypeptide nuclease for administration to a human.

### Treating Diseases in Non-Dividing Cells

In one embodiment, the gene or transcript to be corrected is in a non-dividing cell. Exemplary non-dividing cells are muscle cells or neurons. Non-dividing (especially non-dividing, fully differentiated) cell types present issues for gene targeting or genome engineering, for example because homologous recombination (HR) is generally suppressed in the G1 cell-cycle phase. However, while studying the mechanisms by which cells control normal DNA repair systems, Durocher discovered a previously unknown switch that keeps HR “off” in non-dividing cells and devised a strategy to toggle this switch back on. Orthwein et al. (Daniel Durocher's lab at the Mount Sinai Hospital in Ottawa, Canada) recently reported (Nature 16142, published online 9 Dec. 2015) have shown that the suppression of HR can be lifted and gene targeting successfully concluded in both kidney (293T) and osteosarcoma (U2OS) cells. Tumor suppressors, BRCA1, PALB2 and BRAC2 are known to promote DNA DSB repair by HR. They found that formation of a complex of BRCA1 with PALB2-BRAC2 is governed by a ubiquitin site on PALB2, such that action on the site by an E3 ubiquitin ligase. This E3 ubiquitin ligase is composed of KEAP1 (a PALB2-interacting protein) in complex with cullin-3 (CUL3)-RBX1. PALB2 ubiquitylation suppresses its interaction with BRCA1 and is counteracted by the deubiquitylase USP11, which is itself under cell cycle control. Restoration of the BRCA1-PALB2 interaction combined with the activation of DNA-end resection is sufficient to induce homologous recombination in G1, as measured by a number of methods including a IscB polypeptide nuclease-based gene-targeting assay directed at USP11 or KEAP1 (expressed from a pX459 vector). However, when the BRCA1-PALB2 interaction was restored in resection-competent G1 cells using either KEAP1 depletion or expression of the PALB2-KR mutant, a robust increase in gene-targeting events was detected. These teachings can be adapted for and/or applied to the compositions, systems, described herein.

Thus, reactivation of HR in cells, especially non-dividing, fully differentiated cell types is preferred, In one embodiment. In one embodiment, promotion of the BRCA1-PALB2 interaction is preferred. In one embodiment, the target ell is a non-dividing cell. In one embodiment, the target cell is a neuron or muscle cell. In one embodiment, the target cell is targeted in vivo. In one embodiment, the cell is in G1 and HR is suppressed. In one embodiment, use of KEAP1 depletion, for example inhibition of expression of KEAP1 activity, is preferred. KEAP1 depletion may be achieved through siRNA, for example as shown in Orthwein et al. Alternatively, expression of the PALB2-KR mutant (lacking all eight Lys residues in the BRCA1-interaction domain is preferred, either in combination with KEAP1 depletion or alone. PALB2-KR interacts with BRCA1 irrespective of cell cycle position. Thus, promotion or restoration of the BRCA1-PALB2 interaction, especially in G1 cells, is preferred In one embodiment, especially where the target cells are non-dividing, or where removal and return (ex vivo gene targeting) is problematic, for example neuron or muscle cells. KEAP1 siRNA is available from ThermoFischer. In one embodiment, a BRCA1-PALB2 complex may be delivered to the G1 cell. In one embodiment, PALB2 deubiquitylation may be promoted for example by increased expression of the deubiquitylase USP11, so it is envisaged that a construct may be provided to promote or up-regulate expression or activity of the deubiquitylase USP11.

### Treating Diseases of the Eye

In one embodiment, the disease to be treated is a disease that affects the eyes. Thus, in one embodiment, the composition, system, or component thereof described herein is delivered to one or both eyes.

The composition, system, can be used to correct ocular defects that arise from several genetic mutations further described in Genetic Diseases of the Eye, Second Edition, edited by Elias I. Traboulsi, Oxford University Press, 2012.

In one embodiment, the condition to be treated or targeted is an eye disorder. In one embodiment, the eye disorder may include glaucoma. In one embodiment, the eye disorder includes a retinal degenerative disease. In one embodiment, the retinal degenerative disease is selected from Stargardt disease, Bardet-Biedl Syndrome, Best disease, Blue Cone Monochromacy, Choroidermia, Cone-rod dystrophy, Congenital Stationary Night Blindness, Enhanced S-Cone Syndrome, Juvenile X-Linked Retinoschisis, Leber Congenital Amaurosis, Malattia Leventinesse, Norrie Disease or X-linked Familial Exudative Vitreoretinopathy, Pattern Dystrophy, Sorsby Dystrophy, Usher Syndrome, Retinitis Pigmentosa, Achromatopsia or Macular dystrophies or degeneration, Retinitis Pigmentosa, Achromatopsia, and age related macular degeneration. In one embodiment, the retinal degenerative disease is Leber Congenital Amaurosis (LCA) or Retinitis Pigmentosa. Other exemplary eye diseases are described in greater detail elsewhere herein.

In one embodiment, the composition, system, is delivered to the eye, optionally via intravitreal injection or subretinal injection. Intraocular injections may be performed with the aid of an operating microscope. For subretinal and intravitreal injections, eyes may be prolapsed by gentle digital pressure and fundi visualized using a contact lens system consisting of a drop of a coupling medium solution on the cornea covered with a glass microscope slide coverslip. For subretinal injections, the tip of a 10-mm 34-gauge needle, mounted on a 5-μl Hamilton syringe may be advanced under direct visualization through the superior equatorial sclera tangentially towards the posterior pole until the aperture of the needle was visible in the subretinal space. Then, 2 μl of vector suspension may be injected to produce a superior bullous retinal detachment, thus confirming subretinal vector administration. This approach creates a self-sealing sclerotomy allowing the vector suspension to be retained in the subretinal space until it is absorbed by the RPE, usually within 48 h of the procedure. This procedure may be repeated in the inferior hemisphere to produce an inferior retinal detachment. This technique results in the exposure of approximately 70% of neurosensory retina and RPE to the vector suspension. For intravitreal injections, the needle tip may be advanced through the sclera 1 mm posterior to the corneoscleral limbus and 2 μl of vector suspension injected into the vitreous cavity. For intracameral injections, the needle tip may be advanced through a corneoscleral limbal paracentesis, directed towards the central cornea, and 2 μl of vector suspension may be injected. For intracameral injections, the needle tip may be advanced through a corneoscleral limbal paracentesis, directed towards the central cornea, and 2 μl of vector suspension may be injected. These vectors may be injected at titers of either 1.0-1.4×10¹⁰ or 1.0-1.4×10⁹ transducing units (TU)/ml.

In one embodiment, for administration to the eye, lentiviral vectors. In one embodiment, the lentiviral vector is an equine infectious anemia virus (EIAV) vector. Exemplary EIAV vectors for eye delivery are described in Balagaan, J Gene Med 2006; 8:275-285, Published online 21 Nov. 2005 in Wiley InterScience (www.interscience.wiley.com). DOI: 10.1002/jgm.845; Binley et al., HUMAN GENE THERAPY 23:980-991 (September 2012), which can be adapted for use with the composition, system, described herein. In one embodiment, the dosage can be 1.1×10⁵ transducing units per eye (TU/eye) in a total volume of 100 μl.

Other viral vectors can also be used for delivery to the eye, such as AAV vectors, such as those described in Campochiaro et al., Human Gene Therapy 17:167-176 (February 2006), Millington-Ward et al. (Molecular Therapy, vol. 19 no. 4, 642-649 Apr. 2011; Dalkara et al. (Sci Transl Med 5, 189ra76 (2013)), which can be adapted for use with the composition, system, described herein. In one embodiment, the dose can range from about 106 to 109.5 particle units. In the context of the Millington-Ward AAV vectors, a dose of about 2×10¹¹ to about 6×10¹³ virus particles can be administered. In the context of Dalkara vectors, a dose of about 1×10¹⁵ to about 1×10¹⁶ vg/ml administered to a human.

In one embodiment, the sd-rxRNA® system of RXi Pharmaceuticals may be used/and or adapted for delivering composition, system, to the eye. In this system, a single intravitreal administration of 3 μg of sd-rxRNA results in sequence-specific reduction of PPIB mRNA levels for 14 days. The sd-rxRNA® system may be applied to the nucleic acid-targeting system of the present invention, contemplating a dose of about 3 to 20 mg of composition administered to a human.

In other embodiments, the methods of US Patent Publication No. 20130183282, which is directed to methods of cleaving a target sequence from the human rhodopsin gene, may also be modified to the nucleic acid-targeting system of the present invention.

In other embodiments, the methods of US Patent Publication No. 20130202678 for treating retinopathies and sight-threatening ophthalmologic disorders relating to delivering of the Puf-A gene (which is expressed in retinal ganglion and pigmented cells of eye tissues and displays a unique anti-apoptotic activity) to the sub-retinal or intravitreal space in the eye may be used or adapted. In particular, desirable targets are zgc: 193933, prdmla, spata2, tex 10, rbb4, ddx3, zp2.2, Blimp-1 and HtrA2, all of which may be targeted by the composition, system, of the present invention.

Wu (Cell Stem Cell, 13:659-62, 2013) designed a guide RNA that led Cas9 to a single base pair mutation that causes cataracts in mice, where it induced DNA cleavage. Then using either the other wild-type allele or oligos given to the zygotes repair mechanisms corrected the sequence of the broken allele and corrected the cataract-causing genetic defect in mutant mouse. This approach can be adapted to and/or applied to the compositions, systems, described herein.

US Patent Publication No. 20120159653, describes use of zinc finger nucleases to genetically modify cells, animals and proteins associated with macular degeneration (MD), the teachings of which can be applied to and/or adapted for the compositions, systems, described herein.

One aspect of US Patent Publication No. 20120159653 relates to editing of any chromosomal sequences that encode proteins associated with MD which may be applied to the nucleic acid-targeting system of the present invention.

### Treating Muscle Diseases and Cardiovascular Diseases

In one embodiment, the composition, system can be used to treat and/or prevent a muscle disease and associated circulatory or cardiovascular disease or disorder. The present invention also contemplates delivering the composition, system, described herein, e.g. IsrB polypeptide systems, to the heart. For the heart, a myocardium tropic adeno-associated virus (AAVM) is preferred, in particular AAVM41 which showed preferential gene transfer in the heart (see, e.g., Lin-Yanga et al., PNAS, Mar. 10, 2009, vol. 106, no. 10). Administration may be systemic or local. A dosage of about 1-10×10¹⁴ vector genomes are contemplated for systemic administration. See also, e.g., Eulalio et al. (2012) Nature 492:376 and Somasuntharam et al. (2013) Biomaterials 34:7790, the teachings of which can be adapted for and/or applied to the compositions, systems, described herein.

For example, US Patent Publication No. 20110023139, the teachings of which can be adapted for and/or applied to the compositions, systems, described herein describes use of zinc finger nucleases to genetically modify cells, animals and proteins associated with cardiovascular disease. Cardiovascular diseases generally include high blood pressure, heart attacks, heart failure, and stroke and TIA. Any chromosomal sequence involved in cardiovascular disease or the protein encoded by any chromosomal sequence involved in cardiovascular disease may be utilized in the methods described in this disclosure. The cardiovascular-related proteins are typically selected based on an experimental association of the cardiovascular-related protein to the development of cardiovascular disease. For example, the production rate or circulating concentration of a cardiovascular-related protein may be elevated or depressed in a population having a cardiovascular disorder relative to a population lacking the cardiovascular disorder. Differences in protein levels may be assessed using proteomic techniques including but not limited to Western blot, immunohistochemical staining, enzyme linked immunosorbent assay (ELISA), and mass spectrometry. Alternatively, the cardiovascular-related proteins may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR).

The compositions, systems, herein can be used for treating diseases of the muscular system. The present invention also contemplates delivering the composition, system, described herein, effector protein systems, to muscle(s).

In one embodiment, the muscle disease to be treated is a muscle dystrophy such as DMD. In one embodiment, the composition, system, such as a system capable of RNA modification, described herein can be used to achieve exon skipping to achieve correction of the diseased gene. As used herein, the term “exon skipping” refers to the modification of pre-mRNA splicing by the targeting of splice donor and/or acceptor sites within a pre-mRNA with one or more complementary antisense oligonucleotide(s) (AONs). By blocking access of a spliceosome to one or more splice donor or acceptor site, an AON may prevent a splicing reaction thereby causing the deletion of one or more exons from a fully-processed mRNA. Exon skipping may be achieved in the nucleus during the maturation process of pre-mRNAs. In some examples, exon skipping may include the masking of key sequences involved in the splicing of targeted exons by using a composition, system, described herein capable of RNA modification. In one embodiment, exon skipping can be achieved in dystrophin mRNA. In one embodiment, the composition, system, can induce exon skipping at exon 1, 2, 3, 4, 5, 6, 7, 8, 10, 11, 12, 13, 14, 15, 16, 17, 18, 19, 20, 21, 22, 23, 24, 25, 26, 27, 28, 29, 30, 31, 32, 33, 34, 45, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or any combination thereof of the dystrophin mRNA. In one embodiment, the composition, system, can induce exon skipping at exon 43, 44, 50, 51, 52, 55, or any combination thereof of the dystrophin mRNA. Mutations in these exons, can also be corrected using non-exon skipping polynucleotide modification methods.

In one embodiment, for treatment of a muscle disease, the method of Bortolanza et al. Molecular Therapy vol. 19 no. 11, 2055-264 Nov. 2011) may be applied to an AAV expressing IsrB polypeptide nuclease and injected into humans at a dosage of about 2×10¹⁵ or 2×10¹⁶ vg of vector. The teachings of Bortolanza et al., can be adapted for and/or applied to the compositions, systems, described herein.

In one embodiment, the method of Dumonceaux et al. (Molecular Therapy vol. 18 no. 5, 881-887 May 2010) may be applied to an AAV expressing IsrB polypeptide nuclease and injected into humans, for example, at a dosage of about 10¹⁴ to about 10₁₅ vg of vector. The teachings of Dumonceaux described herein can be adapted for and/or applied to the compositions, systems, described herein.

In one embodiment, the method of Kinouchi et al. (Gene Therapy (2008) 15, 1126-1130) may be applied to compositions described herein and injected into a human, for example, at a dosage of about 500 to 1000 ml of a 40 μM solution into the muscle.

In one embodiment, the method of Hagstrom et al. (Molecular Therapy Vol. 10, No. 2, August 2004) can be adapted for and/or applied to the compositions, systems, herein and injected at a dose of about 15 to about 50 mg into the great saphenous vein of a human.

In one embodiment, the method comprise treating a sickle cell related disease, e.g., sickle cell trait, sickle cell disease such as sickle cell anemia, β-thalassaemia. For example, the method and system may be used to modify the genome of the sickle cell, e.g., by correcting one or more mutations of the β-globin gene. In the case of β-thalassaemia, sickle cell anemia can be corrected by modifying HSCs with the systems. The system allows the specific editing of the cell's genome by cutting its DNA and then letting it repair itself. The IscB polypeptide nuclease is inserted and directed by a RNA guide to the mutated point and then it cuts the DNA at that point. Simultaneously, a healthy version of the sequence is inserted. This sequence is used by the cell's own repair system to fix the induced cut. In this way, the IsrB polypeptide nuclease allows the correction of the mutation in the previously obtained stem cells. The methods and systems may be used to correct HSCs as to sickle cell anemia using a system that targets and corrects the mutation (e.g., with a suitable HDR template that delivers a coding sequence for β-globin, advantageously non-sickling β-globin); specifically, the ωRNA can target mutation that give rise to sickle cell anemia, and the HDR can provide coding for proper expression of β-globin. An ωRNA that targets the mutation-and-IsrB polypeptide nuclease containing particle is contacted with HSCs carrying the mutation. The particle also can contain a suitable HDR template to correct the mutation for proper expression of β-globin; or the HSC can be contacted with a second particle or a vector that contains or delivers the HDR template. The so contacted cells can be administered; and optionally treated/expanded; cf. Cartier. The HDR template can provide for the HSC to express an engineered β-globin gene (e.g., BA-T87Q), or β-globin.

### Treating Diseases of the Liver and Kidney

In one embodiment, the composition, system, or component thereof described herein can be used to treat a disease of the kidney or liver. Thus, in one embodiment, delivery of the composition or component thereof described herein is to the liver or kidney.

Delivery strategies to induce cellular uptake of the therapeutic nucleic acid include physical force or vector systems such as viral-, lipid- or complex-based delivery, or nanocarriers. From the initial applications with less possible clinical relevance, when nucleic acids were addressed to renal cells with hydrodynamic high-pressure injection systemically, a wide range of gene therapeutic viral and non-viral carriers have been applied already to target posttranscriptional events in different animal kidney disease models in vivo (Csaba Révész and Péter Hamar (2011). Delivery Methods to Target RNAs in the Kidney, Gene Therapy Applications, Prof. Chunsheng Kang (Ed.), ISBN: 978-953-307-541-9, InTech, Available from: www.intechopen.com/books/gene-therapy-applications/delivery-methods-to-target-rnas-inthe-kidney). Delivery methods to the kidney may include those in Yuan et al. (Am J Physiol Renal Physiol 295: F605-F617, 2008). The method of Yuang et al. may be applied to the composition of the present invention contemplating a 1-2 g subcutaneous injection of IsrB polypeptide nuclease conjugated with cholesterol to a human for delivery to the kidneys. In one embodiment, the method of Molitoris et al. (J Am Soc Nephrol 20:1754-1764, 2009) can be adapted to the composition and a cumulative dose of 12-20 mg/kg to a human can be used for delivery to the proximal tubule cells of the kidneys. In one embodiment, the methods of Thompson et al. (Nucleic Acid Therapeutics, Volume 22, Number 4, 2012) can be adapted to the compositions and a dose of up to 25 mg/kg can be delivered via i.v. administration. In one embodiment, the method of Shimizu et al. (J Am Soc Nephrol 21:622-633, 2010) can be adapted to the compositions and a dose of about of 10-20 μmol compositions complexed with nanocarriers in about 1-2 liters of a physiologic fluid for i.p. administration can be used.

Other various delivery vehicles can be used to deliver the composition, system to the kidney such as viral, hydrodynamic, lipid, polymer nanoparticles, aptamers and various combinations thereof (see e.g. Larson et al., Surgery, (August 2007), Vol. 142, No. 2, pp. (262-269); Hamar et al., Proc Natl Acad Sci, (October 2004), Vol. 101, No. 41, pp. (14883-14888); Zheng et al., Am J Pathol, (October 2008), Vol. 173, No. 4, pp. (973-980); Feng et al., Transplantation, (May 2009), Vol. 87, No. 9, pp. (1283-1289); Q. Zhang et al., PloS ONE, (July 2010), Vol. 5, No. 7, e11709, pp. (1-13); Kushibikia et al., J Controlled Release, (July 2005), Vol. 105, No. 3, pp. (318-331); Wang et al., Gene Therapy, (July 2006), Vol. 13, No. 14, pp. (1097-1103); Kobayashi et al., Journal of Pharmacology and Experimental Therapeutics, (February 2004), Vol. 308, No. 2, pp. (688-693); Wolfrum et al., Nature Biotechnology, (September 2007), Vol. 25, No. 10, pp. (1149-1157); Molitoris et al., J Am Soc Nephrol, (August 2009), Vol. 20, No. 8 pp. (1754-1764); Mikhaylova et al., Cancer Gene Therapy, (March 2011), Vol. 16, No. 3, pp. (217-226); Y. Zhang et al., J Am Soc Nephrol, (April 2006), Vol. 17, No. 4, pp. (1090-1101); Singhal et al., Cancer Res, (May 2009), Vol. 69, No. 10, pp. (4244-4251); Malek et al., Toxicology and Applied Pharmacology, (April 2009), Vol. 236, No. 1, pp. (97-108); Shimizu et al., J Am Soc Nephrology, (April 2010), Vol. 21, No. 4, pp. (622-633); Jiang et al., Molecular Pharmaceutics, (May-June 2009), Vol. 6, No. 3, pp. (727-737); Cao et al, J Controlled Release, (June 2010), Vol. 144, No. 2, pp. (203-212); Ninichuk et al., Am J Pathol, (March 2008), Vol. 172, No. 3, pp. (628-637); Purschke et al., Proc Natl Acad Sci, (March 2006), Vol. 103, No. 13, pp. (5173-5178).

In one embodiment, delivery is to liver cells. In one embodiment, the liver cell is a hepatocyte. Delivery of the composition and system herein may be via viral vectors, especially AAV (and in particular AAV2/6) vectors. These can be administered by intravenous injection. A preferred target for the liver, whether in vitro or in vivo, is the albumin gene. This is a so-called ‘safe harbor” as albumin is expressed at very high levels and so some reduction in the production of albumin following successful gene editing is tolerated. It is also preferred as the high levels of expression seen from the albumin promoter/enhancer allows for useful levels of correct or transgene production (from the inserted recombination template) to be achieved even if only a small fraction of hepatocytes are edited. See sites identified by Wechsler et al. (reported at the 57th Annual Meeting and Exposition of the American Society of Hematology—abstract available online at ash.confex.com/ash/2015/webprogram/Paper86495.html and presented on 6th December 2015) which can be adapted for use with the compositions, systems, herein.

Exemplary liver and kidney diseases that can be treated and/or prevented are described elsewhere herein.

### Treating Epithelial and Lung Diseases

In one embodiment, the disease treated or prevented by the composition and system described herein can be a lung or epithelial disease. The compositions and systems described herein can be used for treating epithelial and/or lung diseases. The present invention also contemplates delivering the composition, system, described herein, to one or both lungs.

In one embodiment, as viral vector can be used to deliver the composition, system, or component thereof to the lungs. In one embodiment, the AAV is an AAV-1, AAV-2, AAV-5, AAV-6, and/or AAV-9 for delivery to the lungs. (see, e.g., Li et al., Molecular Therapy, vol. 17 no. 12, 2067-277 Dec. 2009). In one embodiment, the MOI can vary from 1×10³ to 4×10⁵ vector genomes/cell. In one embodiment, the delivery vector can be an RSV vector as in Zamora et al. (Am J Respir Crit Care Med Vol 183. pp 531-538, 2011. The method of Zamora et al. may be applied to the nucleic acid-targeting system of the present invention and an aerosolized composition, for example with a dosage of 0.6 mg/kg, may be contemplated for the present invention.

Subjects treated for a lung disease may for example receive pharmaceutically effective amount of aerosolized AAV vector system per lung endobronchially delivered while spontaneously breathing. As such, aerosolized delivery is preferred for AAV delivery in general. An adenovirus or an AAV particle may be used for delivery. Suitable gene constructs, each operably linked to one or more regulatory sequences, may be cloned into the delivery vector. In this instance, the following constructs are provided as examples: Cbh or EF1α promoter for Cas, U6 or H1 promoter for ωRNA),: A preferred arrangement is to use a CFTRdelta508 targeting guide, a repair template for deltaF508 mutation and a codon optimized composition, with optionally one or more nuclear localization signal or sequence(s) (NLS(s)), e.g., two (2) NLSs.

### Treating Diseases of the Skin

The compositions and systems described herein can be used for the treatment of skin diseases. The present invention also contemplates delivering the composition and system, described herein, to the skin.

In one embodiment, delivery to the skin (intradermal delivery) of the composition, system, or component thereof can be via one or more microneedles or microneedle containing device. For example, In one embodiment the device and methods of Hickerson et al. (Molecular Therapy-Nucleic Acids (2013) 2, e129) can be used and/or adapted to deliver the composition, system, described herein, for example, at a dosage of up to 300 μl of 0.1 mg/ml compositions to the skin.

In one embodiment, the methods and techniques of Leachman et al. (Molecular Therapy, vol. 18 no. 2, 442-446 Feb. 2010) can be used and/or adapted for delivery of a compositions described herein to the skin.

In one embodiment, the methods and techniques of Zheng et al. (PNAS, Jul. 24, 2012, vol. 109, no. 30, 11975-11980) can be used and/or adapted for nanoparticle delivery of a compositions described herein to the skin. In one embodiment, as dosage of about 25 nM applied in a single application can achieve gene knockdown in the skin.

### Treating Cancer

The compositions, systems, described herein can be used for the treatment of cancer. The present invention also contemplates delivering the composition, system, described herein, to a cancer cell. Also, as is described elsewhere herein the compositions, systems, can be used to modify an immune cell, such as a CAR or CAR T cell, which can then in turn be used to treat and/or prevent cancer. This is also described in International Patent Publication No. WO 2015/161276, the disclosure of which is hereby incorporated by reference and described herein below.

Target genes suitable for the treatment or prophylaxis of cancer can include those set forth in Tables 7 and 8. In one embodiment, target genes for cancer treatment and prevention can also include those described in International Patent Publication No. WO 2015/048577 the disclosure of which is hereby incorporated by reference and can be adapted for and/or applied to the composition, system, described herein.

### Adoptive Cell Therapy

The compositions, systems, and components thereof described herein can be used to modify cells for an adoptive cell therapy. In an aspect of the invention, methods and compositions which involve editing a target nucleic acid sequence, or modulating expression of a target nucleic acid sequence, and applications thereof in connection with cancer immunotherapy are comprehended by adapting the composition, system, of the present invention. In some examples, the compositions, systems, and methods may be used to modify a stem cell (e.g., induced pluripotent cell) to derive modified natural killer cells, gamma delta T cells, and alpha beta T cells, which can be used for the adoptive cell therapy. In certain examples, the compositions, systems, and methods may be used to modify modified natural killer cells, gamma delta T cells, and alpha beta T cells.

As used herein, “ACT”, “adoptive cell therapy” and “adoptive cell transfer” may be used interchangeably. In an embodiment, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for-thalassemia, Nat Commun. 2017 Sep. 4; 8 (1): 424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June; 24 (6): 724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314 (5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In an embodiment, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32:189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12 (4): 269-281; and Jenson and Riddell, 2014, Design and implementation of adoptive therapy with chimeric antigen receptor-modified T cells. Immunol Rev. 257 (1): 127-144; and Rajasagi et al., 2014, Systematic identification of personal tumor-specific neoantigens in chronic lymphocytic leukemia. Blood. 2014 Jul. 17; 124 (3): 453-62).

In an embodiment, an antigen (such as a tumor antigen) to be targeted in adoptive cell therapy (such as particularly CAR or TCR T-cell therapy) of a disease (such as particularly of tumor or cancer) may be selected from a group consisting of: MRI (see, e.g., Crowther, et al., 2020, Genome-wide CRISPR-Cas9 screening reveals ubiquitous T cell cancer targeting via the monomorphic MHC class I-related protein MRI, Nature Immunology vol. 21, pages 178-185), B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); K-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTAI (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (ICE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac (2-8) aNeu5Ac (2-3) bDGalp (1-4) bDGlcp (1-1) Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis (Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac (2-3)bDGalp(1-4)bDGlcp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein (N-250); GAGE (G antigen); GnT-V acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); LICAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In an embodiment, an antigen to be targeted in adoptive cell therapy (such as particularly CAR or TCR T-cell therapy) of a disease (such as particularly of tumor or cancer) is a tumor-specific antigen (TSA).

In an embodiment, an antigen to be targeted in adoptive cell therapy (such as particularly CAR or TCR T-cell therapy) of a disease (such as particularly of tumor or cancer) is a neoantigen.

In an embodiment, an antigen to be targeted in adoptive cell therapy (such as particularly CAR or TCR T-cell therapy) of a disease (such as particularly of tumor or cancer) is a tumor-associated antigen (TAA).

In an embodiment, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (DI), and any combinations thereof.

In an embodiment, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2.

In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CML), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR a and B chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO 9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, In one embodiment, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CD5, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8a hinge domain and a CD8a transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD32; see U.S. Pat. Nos. 8,911,993; 8,916,381; 8,975,071; 9,101,584; 9,102,760; 9,102,761). Third-generation CARs include a combination of costimulatory endodomains, such a CD35-chain, CD97, GDI 1a-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD32; see U.S. Pat. Nos. 8,906,682; 8,399,645; 5,686,281; PCT Publication No. WO 2014/134165; PCT Publication No. WO 2012/079000). In an embodiment, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon R1b), CD79a, CD79b, Fc gamma RIIa, DAP10, and DAP12. In certain preferred embodiments, the primary signaling domain comprises a functional signaling domain of CD3ζ or FcRγ. In an embodiment, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In an embodiment, the one or more costimulatory signaling domains comprise a functional signaling domain of a protein selected, each independently, from the group consisting of: 4-1BB, CD27, and CD28. In an embodiment, a chimeric antigen receptor may have the design as described in U.S. Pat. No. 7,446,190, comprising an intracellular domain of CD3ζ chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139. Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, an embodiment employ a CAR comprising (a) a zeta chain portion comprising the intracellular domain of human CD3 chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects.

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34:1157-1165), a portion of the human CD28 molecule, and the intracellular component of the human TCR-(molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-¿ molecule. The exact sequence of the CD28 molecule included in the FMC63-28Z CAR corresponded to Genbank identifier NM_006139; the sequence included all amino acids starting with the amino acid sequence IEVMYPPPY (SEQ ID NO: 1565) and continuing all the way to the carboxy-terminus of the protein. To encode the anti-CD19 scFv component of the vector, the authors designed a DNA sequence which was based on a portion of a previously published CAR (Cooper et al., (2003) Blood 101:1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16:457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20:70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in an embodiment, cells intended for adoptive cell therapies, more particularly immunoresponsive cells such as T cells, may express the FMC63-28Z CAR as described by Kochenderfer et al. (supra). Hence, in an embodiment, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3 chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY (SEQ ID NO: 1565) and continuing all the way to the carboxy-terminus of the protein. Preferably, the antigen is CD19, more preferably the antigen-binding element is an anti-CD19 scFv, even more preferably the anti-CD19 scFv as described by Kochenderfer et al. (supra).

Additional anti-CD19 CARS are further described in International Patent Publication No. WO 2015/187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signaling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-Fc RI gamma chain; or CD28-Fc RI gamma chain) were disclosed. Hence, in an embodiment, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signaling domain as set forth in Table 1 of No. WO 2015/187528. Preferably, the antigen is CD19, more preferably the antigen-binding element is an anti-CD19 scFv, even more preferably the mouse or human anti-CD 19 scFv as described in Example 1 of. WO 2015/187528. In an embodiment, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20 (1): 55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am. J. Pathol. 1995; 147:1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In an embodiment, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In an embodiment, the chimeric inhibitory receptor comprises an extracellular antigen-binding element (or portion or domain) configured to specifically bind to a target antigen, a transmembrane domain, and an intracellular immunosuppressive or repressive signaling domain. In an embodiment, the second target antigen is an antigen that is not expressed on the surface of a cancer cell or infected cell or the expression of which is downregulated on a cancer cell or an infected cell. In an embodiment, the second target antigen is an MHC-class I molecule. In an embodiment, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in one embodiment, TCR expression may eliminated using RNA interference (e.g., shRNA, siRNA, miRNA, etc.), IscB polypeptide or CRISPR-associated IscB polypeptide nuclease, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US Patent Publication Nos. US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLOS One, 2013) or to downregulate expression of the CAR following binding to the target antigen (International Patent Publication No. WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In an embodiment, ACT includes co-transferring CD4+ Th1 cells and CD8+ CTLs to induce a synergistic antitumor response (see, e.g., Li et al., Adoptive cell therapy with CD4+ T helper 1 cells and CD8+ cytotoxic T cells enhances complete rejection of an established tumor, leading to generation of endogenous memory responses to non-targeted tumor epitopes. Clin Transl Immunology. 2017 October; 6 (10): e160).

In an embodiment, Th 17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112 (2): 362-73; and Martin-Orozco N, et al., T helper 17 cells promote cytotoxic T cell activation in tumor immunity. Immunity. 2009 Nov. 20; 31 (5): 787-98). Those studies involved an adoptive T cell transfer (ACT) therapy approach, which takes advantage of CD4+ T cells that express a TCR recognizing tyrosinase tumor antigen. Exploitation of the TCR leads to rapid expansion of Th17 populations to large numbers ex vivo for reinfusion into the autologous tumor-bearing hosts.

In an embodiment, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In an embodiment, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257 (1): 56-71. doi: 10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In an embodiment, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3 (10): 1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells, or population of cells, may be made resistant to at least one immunosuppressive agent due to the inactivation of a gene encoding a receptor for such immunosuppressive agent. In an embodiment, the immunosuppressive treatment provides for the selection and expansion of the immunoresponsive T cells within the patient.

In an embodiment, the treatment can be administered before primary treatment (e.g., surgery or radiation therapy) to shrink a tumor before the primary treatment. In another embodiment, the treatment can be administered after primary treatment to remove any remaining cancer cells.

In an embodiment, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In one embodiment, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection.

The administration of the cells or population of cells can consist of the administration of 104-109 cells per kg body weight, preferably 105 to 106 cells/kg body weight including all integer values of cell numbers within those ranges. Dosing in CAR T cell therapies may for example involve administration of from 106 to 109 cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6:95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; International Patent Publication WO 2011/146862; International Patent Publication WO 2014/011987; International Patent Publication WO 2013/040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28 (6): 1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23 (9): 2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 Jan. 25; 9 (374); Legut, et al., 2018, CRISPR-mediated TCR replacement generates superior anticancer transgenic T cells. Blood, 131 (3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. The composition and systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see International Patent Publication Nos. WO 2013/176915, WO 2014/059173, WO 2014/172606, WO 2014/184744, and WO 2014/191128).

In an embodiment, editing may result in inactivation of a gene. By inactivating a gene, it is intended that the gene of interest is not expressed in a functional protein form. In a particular embodiment, the system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In an embodiment, homology directed repair (HDR) is used to concurrently inactivate a gene (e.g., TRAC) and insert an endogenous TCR or CAR into the inactivated locus.

Hence, in an embodiment, editing of cells, particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543:113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V (D) J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in an embodiment, editing of cells, particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as IscB system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112 (12): 4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In an embodiment, editing of cells, particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In an embodiment, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44 (2): 356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

International Patent Publication No. WO 2014/172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In an embodiment, metallothioneins are targeted by gene editing in adoptively transferred T cells.

In an embodiment, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10ORB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, International Patent Publication No. WO 2016/196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as the composition or system herein) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In an embodiment, cells may be engineered to express a CAR, wherein expression and/or function of methylcytosine dioxygenase genes (TET1, TET2 and/or TET3) in the cells has been reduced or eliminated, (such as the composition or system herein) (for example, as described in WO201704916).

In an embodiment, editing of cells, particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In an embodiment, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in International Patent Publication Nos. WO 2016/011210 and WO 2017/011804).

In an embodiment, editing of cells, particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In an embodiment, a cell may be multiplied edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In one embodiment, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In an embodiment of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (e.g., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In an embodiment, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In an embodiment, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead: cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In an embodiment, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×106/ml. In other embodiments, the concentration used can be from about 1×105/ml to 1×106/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In an embodiment, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274 (5284): 94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of 1251 labeled B2-microglobulin (B2m) into MHC class I/B2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003/057171, U.S. Pat. No. 8,034,334, and U.S. Patent Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In an embodiment, T cells comprising a CAR or an exogenous TCR, may be manufactured as described in International Patent Publication No. WO 2015/120096, by a method comprising enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In an embodiment, T cells comprising a CAR or an exogenous TCR, may be manufactured as described in WO 2015/120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In an embodiment, T cell maturation or differentiation in vitro may be delayed or inhibited by the method as described in International Patent Publication No. WO 2017/070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In an embodiment, a patient in need of a T cell therapy may be conditioned by a method as described in International Patent Publication No. WO 2016/191756 comprising administering to the patient a dose of cyclophosphamide between 200 mg/m2/day and 2000 mg/m2/day and a dose of fludarabine between 20 mg/m2/day and 900 mg/m2/day.

### Diseases

Genetic Diseases and Diseases with a Genetic and/or Epigenetic Aspect

The compositions, systems, or components thereof can be used to treat and/or prevent a genetic disease or a disease with a genetic and/or epigenetic aspect. The genes and conditions exemplified herein are not exhaustive. In one embodiment, a method of treating and/or preventing a genetic disease can include administering a composition, system, and/or one or more components thereof to a subject, where the composition, system, and/or one or more components thereof is capable of modifying one or more copies of one or more genes associated with the genetic disease or a disease with a genetic and/or epigenetic aspect in one or more cells of the subject. In one embodiment, modifying one or more copies of one or more genes associated with a genetic disease or a disease with a genetic and/or epigenetic aspect in the subject can eliminate a genetic disease or a symptom thereof in the subject. In one embodiment, modifying one or more copies of one or more genes associated with a genetic disease or a disease with a genetic and/or epigenetic aspect in the subject can decrease the severity of a genetic disease or a symptom thereof in the subject. In one embodiment, the compositions, systems, or components thereof can modify one or more genes or polynucleotides associated with one or more diseases, including genetic diseases and/or those having a genetic aspect and/or epigenetic aspect, including but not limited to, any one or more set forth in Table 4. It will be appreciated that those diseases and associated genes listed herein are non-exhaustive and non-limiting. Further some genes play roles in the development of multiple diseases.

TABLE 4
Exemplary Genetic and Other Diseases and Associated Genes

| | Primary | | |
|---|---|---|---|
| | Tissues or | Additional | |
| | System | Tissues/Systems | |
| Disease Name | Affected | Affected | Genes |
| Achondroplasia | Bone and | | fibroblast growth factor receptor 3 |
| | Muscle | | (FGFR3) |
| Achromatopsia | eye | | CNGA3, CNGB3, GNAT2, PDE6C, |
| | | | PDE6H, ACHM2, ACHM3, |
| Acute Renal Injury | kidney | | NFkappaB, AATF, p85alpha, FAS, |
| | | | Apoptosis cascade elements (e.g. |
| | | | FASR, Caspase 2, 3, 4, 6, 7, 8, 9, 10, |
| | | | AKT, TNF alpha, IGF1, IGF1R, |
| | | | RIPK1), p53 |
| Age Related Macular | eye | | Abcr; CCL2; CC2; CP |
| Degeneration | | | (ceruloplasmin); Timp3; cathepsinD; |
| | | | VLDLR, CCR2 |
| AIDS | Immune System | | KIR3DL1, NKAT3, NKB1, AMB11, |
| | | | KIR3DS1, IFNG, CXCL12, SDF1 |
| Albinism (including | Skin, hair, eyes, | | TYR, OCA2, TYRP1, and SLC45A2, |
| oculocutaneous albinism (types | | | SLC24A5 and C10orf11 |
| 1-7) and ocular albinism) | | | |
| Alkaptonuria | Metabolism of | Tissues/organs | HGD |
| | amino acids | where | |
| | | homogentisic acid | |
| | | accumulates, | |
| | | particularly | |
| | | cartilage (joints), | |
| | | heart valves, | |
| | | kidneys | |
| alpha-1 antitrypsin deficiency | Lung | Liver, skin, | SERPINA1, those set forth in |
| (AATD or A1AD) | | vascular system, | WO2017165862, PiZ allele |
| | | kidneys, GI | |
| ALS | CNS | | SOD1; ALS2; ALS3; ALS5; |
| | | | ALS7; STEX; FUS; TARDBP; VEGF |
| | | | (VEGF-a; |
| | | | VEGF-b; VEGF-c); DPP6; NEFH, |
| | | | PTGS1, SLC1A2, TNFRSF10B, |
| | | | PRPH, HSP90AA1, CRIA2, IFNG, |
| | | | AMPA2 S100B, FGF2, AOX1, CS, |
| | | | TXN, RAPHJ1, MAP3K5, NBEAL1, |
| | | | GPX1, ICA1L, RAC1, MAPT, |
| | | | ITPR2, ALS2CR4, GLS, ALS2CR8, |
| | | | CNTFR, ALS2CR11, FOLH1, |
| | | | FAM117B, P4HB, CNTF, SQSTM1, |
| | | | STRADB, NAIP, NLR, YWHAQ, |
| | | | SLC33A1, TRAK2, SCA1, NIF3L1, |
| | | | NIF3, PARD3B, COX8A, CDK15, |
| | | | HECW1, HECT, C2, WW 15, NOS1, |
| | | | MET, SOD2, HSPB1, NEFL, CTSB, |
| | | | ANG, HSPA8, RNase A, VAPB, |
| | | | VAMP, SNCA, alpha HGF, CAT, |
| | | | ACTB, NEFM, TH, BCL2, FAS, |
| | | | CASP3, CLU, SMN1, G6PD, BAX, |
| | | | HSF1, RNF19A, JUN, ALS2CR12, |
| | | | HSPA5, MAPK14, APEX1, |
| | | | TXNRD1, NOS2, TIMP1, CASP9, |
| | | | XIAP, GLG1, EPO, VEGFA, ELN, |
| | | | GDNF, NFE2L2, SLC6A3, HSPA4, |
| | | | APOE, PSMB8, DCTN2, TIMP3, |
| | | | KIFAP3, SLC1A1, SMN2, CCNC, |
| | | | STUB1, ALS2, PRDX6, SYP, |
| | | | CABIN1, CASP1, GART, CDK5, |
| | | | ATXN3, RTN4, C1QB, VEGFC, |
| | | | HTT, PARK7, XDH, GFAP, MAP2, |
| | | | CYCS, FCGR3B, CCS, UBL5, |
| | | | MMP9m SLC18A3, TRPM7, |
| | | | HSPB2, AKT1, DEERL1, CCL2, |
| | | | NGRN, GSR, TPPP3, APAF1, |
| | | | BTBD10, GLUD1, CXCR4, S: C1A3, |
| | | | FLT1, PON1, AR, LIF, ERBB3, :GA: S1, |
| | | | CD44, TP53, TLR3, GRIA1, |
| | | | GAPDH, AMPA, GRIK1, DES, |
| | | | CHAT, FLT4, CHMP2B, BAG1, |
| | | | CHRNA4, GSS, BAK1, KDR, |
| | | | GSTP1, OGG1, IL6 |
| Alzheimer's Disease | Brain | | E1; CHIP; UCH; UBB; Tau; LRP; |
| | | | PICALM; CLU; PS1; |
| | | | SORL1; CR1; VLDLR; UBA1; |
| | | | UBA3; CHIP28; AQP1; UCHL1; |
| | | | UCHL3; APP, AAA, CVAP, AD1, |
| | | | APOE, AD2, DCP1, ACE1, MPO, |
| | | | PACIP1, PAXIP1L, PTIP, A2M, |
| | | | BLMH, BMH, PSEN1, AD3, |
| | | | ALAS2, ABCA1, BIN1, BDNF, |
| | | | BTNL8, C1ORF49, CDH4, |
| | | | CHRNB2, CKLFSF2, CLEC4E, |
| | | | CR1L, CSF3R, CST3, CYP2C, |
| | | | DAPK1, ESR1, FCAR, FCGR3B, |
| | | | FFA2, FGA, GAB2, GALP, |
| | | | GAPDHS, GMPB, HP, HTR7, IDE, |
| | | | IF127, IFI6, IFIT2, IL1RN, IL-IRA, |
| | | | IL8RA, IL8RB, JAG1, KCNJ15, |
| | | | LRP6, MAPT, MARK4, |
| | | | MPHOSPH1, MTHFR, NBN, |
| | | | NCSTN, NIACR2, NMNAT3, NTM, |
| | | | ORM1, P2RY13, PBEF1, PCK1, |
| | | | PICALM, PLAU, PLXNC1, PRNP, |
| | | | PSEN1, PSEN2, PTPRA, RALGPS2, |
| | | | RGSL2, SELENBP1, SLC25A37, |
| | | | SORL1, Mitoferrin-1, TF, TFAM, |
| | | | TNF, TNFRSF10C, UBE1C |
| Amyloidosis | | | APOA1, APP, AAA, CVAP, AD1, |
| | | | GSN, FGA, LYZ, TTR, PALB |
| Amyloid neuropathy | | | TTR, PALB |
| Anemia | Blood | | CDAN1, CDA1, RPS19, DBA, |
| | | | PKLR, PK1, NT5C3, UMPH1, PSN1, |
| | | | RHAG, RH50A, NRAMP2, SPTB, |
| | | | ALAS2, ANH1, ASB, ABCB7, |
| | | | ABC7, ASAT |
| Angelman Syndrome | Nervous system, | | UBE3A |
| | brain | | |
| Attention Deficit Hyperactivity | Brain | | PTCHD1 |
| Disorder (ADHD) | | | |
| Autoimmune | Immune system | | TNFRSF6, APT1, FAS, CD95, |
| lymphoproliferative syndrome | | | ALPS1A |
| Autism, Autism spectrum | Brain | | PTCHD1; Mecp2; BZRAP1; |
| disorders (ASDs), including | | | MDGA2; Sema5A; Neurexin 1; |
| Asperger's and a general | | | GLO1, RTT, PPMX, MRX16, RX79, |
| diagnostic category called | | | NLGN3, NLGN4, KIAA1260, |
| Pervasive Developmental | | | AUTSX2, FMR1, FMR2; FXR1; |
| Disorders (PDDs) | | | FXR2; MGLUR5, ATP10C, CDH10, |
| | | | GRM6, MGLUR6, CDH9, CNTN4, |
| | | | NLGN2, CNTNAP2, SEMA5A, |
| | | | DHCR7, NLGN4X, NLGN4Y, DPP6, |
| | | | NLGN5, EN2, NRCAM, MDGA2, |
| | | | NRXN1, FMR2, AFF2, FOXP2, |
| | | | OR4M2, OXTR, FXR1, FXR2, PAH, |
| | | | GABRA1, PTEN, GABRA5, |
| | | | PTPRZ1, GABRB3, GABRG1, |
| | | | HIRIP3, SEZ6L2, HOXA1, |
| | | | SHANK3, IL6, SHBZRAP1, |
| | | | LAMB1, SLC6A4, SERT, MAPK3, |
| | | | TAS2R1, MAZ, TSC1, MDGA2, |
| | | | TSC2, MECP2, UBE3A, WNT2, see |
| | | | also 20110023145 |
| autosomal dominant polycystic | kidney | liver | PKD1, PKD2 |
| kidney disease (ADPKD) - | | | |
| (includes diseases such as von | | | |
| Hippel-Lindau disease and | | | |
| tubreous sclerosis complex | | | |
| disease) | | | |
| Autosomal Recessive Polycystic | kidney | liver | PKDH1 |
| Kidney Disease (ARPKD) | | | |
| Ataxia-Telangiectasia (a.k.a | Nervous system, | various | ATM |
| Louis Bar syndrome) | immune system | | |
| B-Cell Non-Hodgkin | | | BCL7A, BCL7 |
| Lymphoma | | | |
| Bardet-Biedl syndrome | Eye, | Liver, ear, | ARL6, BBS1, BBS2, BBS4, BBS5, |
| | musculoskeletal | gastrointestinal | BBS7, BBS9, BBS10, BBS12, |
| | system, kidney, | system, brain | CEP290, INPP5E, LZTFL1, MKKS, |
| | reproductive | | MKS1, SDCCAG8, TRIM32, TTC8 |
| | organs | | |
| Bare Lymphocyte Syndrome | blood | | TAPBP, TPSN, TAP2, ABCB3, |
| | | | PSF2, RING11, MHC2TA, C2TA, |
| | | | RFX5, RFXAP, RFX5 |
| Bartter's Syndrome (types I, II, | kidney | | SLC12A1 (type I), KCNJ1 (type II), |
| III, IVA and B, and V) | | | CLCNKB (type III), BSND (type IV |
| | | | A), or both the CLCNKA CLCNKB |
| | | | genes (type IV B), CASR (type V). |
| Becker muscular dystrophy | Muscle | | DMD, BMD, MYF6 |
| Best Disease (Vitelliform | eye | | VMD2 |
| Macular Dystrophy type 2) | | | |
| Bleeding Disorders | blood | | TBXA2R, P2RX1, P2X1 |
| Blue Cone Monochromacy | eye | | OPN1LW, OPN1MW, and LCR |
| Breast Cancer | Breast tissue | | BRCA1, BRCA2, COX-2 |
| Bruton's Disease (aka X-linked | Immune system, | | BTK |
| Agammglobulinemia) | specifically B | | |
| | cells | | |
| Cancers (e.g., lymphoma, | Various | | FAS, BID, CTLA4, PDCD1, CBLB, |
| chronic lymphocytic leukemia | | | PTPN6, TRAC, TRBC, those |
| (CLL), B cell acute lymphocytic | | | described in WO2015048577 |
| leukemia (B-ALL), acute | | | |
| lymphoblastic leukemia, acute | | | |
| myeloid leukemia, non- | | | |
| Hodgkin's lymphoma (NHL), | | | |
| diffuse large cell lymphoma | | | |
| (DLCL), multiple myeloma, | | | |
| renal cell carcinoma (RCC), | | | |
| neuroblastoma, colorectal | | | |
| cancer, breast cancer, ovarian | | | |
| cancer, melanoma, sarcoma, | | | |
| prostate cancer, lung cancer, | | | |
| esophageal cancer, | | | |
| hepatocellular carcinoma, | | | |
| pancreatic cancer, astrocytoma, | | | |
| mesothelioma, head and neck | | | |
| cancer, and medulloblastoma | | | |
| Cardiovascular Diseases | heart | Vascular system | IL1B, XDH, TP53, PTGS, MB, IL4, |
| | | | ANGPT1, ABCGu8, CTSK, PTGIR, |
| | | | KCNJ11, INS, CRP, PDGFRB, |
| | | | CCNA2, PDGFB, KCNJ5, KCNN3, |
| | | | CAPN10, ADRA2B, ABCG5, |
| | | | PRDX2, CPAN5, PARP14, MEX3C, |
| | | | ACE, RNF, IL6, TNF, STN, |
| | | | SERPINE1, ALB, ADIPOQ, APOB, |
| | | | APOE, LEP, MTHFR, APOA1, |
| | | | EDN1, NPPB, NOS3, PPARG, |
| | | | PLAT, PTGS2, CETP, AGTR1, |
| | | | HMGCR, IGF1, SELE, REN, |
| | | | PPARA, PON1, KNG1, CCL2, LPL, |
| | | | VWF, F2, ICAM1, TGFB, NPPA, |
| | | | IL10, EPO, SOD1, VCAM1, IFNG, |
| | | | LPA, MPO, ESR1, MAPK, HP, F3, |
| | | | CST3, COG2, MMP9, SERPINC1, |
| | | | F8, HMOX1, APOC3, IL8, PROL1, |
| | | | CBS, NOS2, TLR4, SELP, ABCA1, |
| | | | AGT, LDLR, GPT, VEGFA, NR3C2, |
| | | | IL18, NOS1, NR3C1, FGB, HGF, |
| | | | IL1A, AKT1, LIPC, HSPD1, |
| | | | MAPK14, SPP1, ITGB3, CAT, |
| | | | UTS2, THBD, F10, CP, |
| | | | TNFRSF11B, EGFR, MMP2, PLG, |
| | | | NPY, RHOD, MAPK8, MYC, FN1, |
| | | | CMA1, PLAU, GNB3, ADRB2, |
| | | | SOD2, F5, VDR, ALOX5, HLA- |
| | | | DRB1, PARP1, CD40LG, PON2, |
| | | | AGER, IRS1, PTGS1, ECE1, F7, |
| | | | IRMN, EPHX2, IGFBP1, MAPK10, |
| | | | FAS, ABCB1, JUN, IGFBP3, CD14, |
| | | | PDE5A, AGTR2, CD40, LCAT, |
| | | | CCR5, MMP1, TIMP1, ADM, |
| | | | DYT10, STAT3, MMP3, ELN, |
| | | | USF1, CFH, HSPA4, MMP12, MME, |
| | | | F2R, SELL, CTSB, ANXA5, |
| | | | ADRB1, CYBA, FGA, GGT1, LIPG, |
| | | | HIF1A, CXCR4, PROC, SCARB1, |
| | | | CD79A, PLTP, ADD1, FGG, SAA1, |
| | | | KCNH2, DPP4, NPR1, VTN, |
| | | | KIAA0101, FOS, TLR2, PPIG, |
| | | | IL1R1, AR, CYP1A1, SERPINA1, |
| | | | MTR, RBP4, APOA4, CDKN2A, |
| | | | FGF2, EDNRB, ITGA2, VLA-2, |
| | | | CABIN1, SHBG, HMGB1, |
| | | | HSP90B2P, CYP3A4, GJA1, CAV1, |
| | | | ESR2, LTA, GDF15, BDNF, |
| | | | CYP2D6, NGF, SP1, TGIF1, SRC, |
| | | | EGF, PIK3CG, HLA-A, KCNQ1, |
| | | | CNR1, FBN1, CHKA, BEST1, |
| | | | CTNNB1, IL2, CD36, PRKAB1, |
| | | | TPO, ALDH7A1, CX3CR1, TH, F9, |
| | | | CH1, TF, HFE, IL17A, PTEN, |
| | | | GSTM1, DMD, GATA4, F13A1, |
| | | | TTR, FABP4, PON3, APOC1, INSR, |
| | | | TNFRSF1B, HTR2A, CSF3, |
| | | | CYP2C9, TXN, CYP11B2, PTH, |
| | | | CSF2, KDR, PLA2G2A, THBS1, |
| | | | GCG, RHOA, ALDH2, TCF7L2, |
| | | | NFE2L2, NOTCH1, UGT1A1, |
| | | | IFNA1, PPARD, SIRT1, GNHR1, |
| | | | PAPPA, ARR3, NPPC, AHSP, |
| | | | PTK2, IL 13, MTOR, ITGB2, GSTT1, |
| | | | IL6ST, CPB2, CYP1A2, HNF4A, |
| | | | SLC64A, PLA2G6, TNFSF11, |
| | | | SLC8A1, F2RL1, AKR1A1, |
| | | | ALDH9A1, BGLAP, MTTP, MTRR, |
| | | | SULT1A3, RAGE, C4B, P2RY12, |
| | | | RNLS, CREB1, POMC, RAC1, |
| | | | LMNA, CD59, SCM5A, CYP1B1, |
| | | | MIF, MMP13, TIMP2, CYP19A1, |
| | | | CUP21A2, PTPN22, MYH14, MBL2, |
| | | | SELPLG, AOC3, CTSL1, PCNA, |
| | | | IGF2, ITGB1, CAST, CXCL12, |
| | | | IGHE, KCNE1, TFRC, COL1A1, |
| | | | COL1A2, IL2RB, PLA2G10, |
| | | | ANGPT2, PROCR, NOX4, HAMP, |
| | | | PTPN11, SLCA1, IL2RA, CCL5, |
| | | | IRF1, CF: AR, CA: CA, EIF4E, |
| | | | GSTP1, JAK2, CYP3A5, HSPG2, |
| | | | CCL3, MYD88, VIP, SOAT1, |
| | | | ADRBK1, NR4A2, MMP8, NPR2, |
| | | | GCH1, EPRS, PPARGC1A, F12, |
| | | | PECAM1, CCL4, CERPINA34, |
| | | | CASR, FABP2, TTF2, PROS1, |
| | | | CTF1, SGCB, YME1L1, CAMP, |
| | | | ZC3H12A, AKR1B1, MMP7, AHR, |
| | | | CSF1, HDAC9, CTGF, KCNMA1, |
| | | | UGT1A, PRKCA, COMT, S100B, |
| | | | EGR1, PRL, IL15, DRD4, CAMK2G, |
| | | | SLC22A2, CCL11, PGF, THPO, |
| | | | GP6, TACR1, NTS, HNF1A, SST, |
| | | | KCDN1, LOC646627, TBXAS1, |
| | | | CUP2J2, TBXA2R, ADH1C, |
| | | | ALOX12, AHSG, BHMT, GJA4, |
| | | | SLC25A4, ACLY, ALOX5AP, |
| | | | NUMA1, CYP27B1, CYSLTR2, |
| | | | SOD3, LTC4S, UCN, GHRL, |
| | | | APOC2, CLEC4A, KBTBD10, TNC, |
| | | | TYMS, SHC1, LRP1, SOCS3, |
| | | | ADH1B, KLK3, HSD11B1, |
| | | | VKORC1, SERPINB2, TNS1, |
| | | | RNF19A, EPOR, ITGAM, PITX2, |
| | | | MAPK7, FCGR3A, LEEPR, ENG, |
| | | | GPX1, GOT2, HRH1, NR112, CRH, |
| | | | HTR1A, VDAC1, HPSE, SFTPD, |
| | | | TAP2, RMF123, PTK2Bm NTRK2, |
| | | | IL6R, ACHE, GLP1R, GHR, GSR, |
| | | | NQO1, NR5A1, GJB2, SLC9A1, |
| | | | MAOA, PCSK9, FCGR2A, |
| | | | SERPINF1, EDN3, UCP2, TFAP2A, |
| | | | C4BPA, SERPINF2, TYMP, ALPP, |
| | | | CXCR2, SLC3A3, ABCG2, ADA, |
| | | | JAK3, HSPA1A, FASN, FGF1, F11, |
| | | | ATP7A, CR1, GFPA, ROCK1, |
| | | | MECP2, MYLK, BCHE, LIPE, |
| | | | ADORA1, WRN, CXCR3, CD81, |
| | | | SMAD7, LAMC2, MAP3K5, CHGA, |
| | | | IAPP, RHO, ENPP1, PTHLH, NRG1, |
| | | | VEGFC, ENPEP, CEBPB, NAGLU,. |
| | | | F2RL3, CX3CL1, BDKRB1, |
| | | | ADAMTS13, ELANE, ENPP2, |
| | | | CISH, GAST, MYOC, ATP1A2, |
| | | | NF1, GJB1, MEF2A, VCL, BMPR2, |
| | | | TUBB, CDC42, KRT18, HSF1, |
| | | | MYB, PRKAA2, ROCK2, TFP1, |
| | | | PRKG1, BMP2, CTNND1, CTH, |
| | | | CTSS, VAV2, NPY2R, IGFBP2, |
| | | | CD28, GSTA1, PPIA, APOH, |
| | | | S100A8, IL11, ALOX15, FBLN1, |
| | | | NR1H3, SCD, GIP, CHGB, PRKCB, |
| | | | SRD5A1, HSD11B2, CALCRL, |
| | | | GALNT2, ANGPTL4, KCNN4, |
| | | | PIK3C2A, HBEGF, CYP7A1, HLA- |
| | | | DRB5, BNIP3, GCKR, S100A12, |
| | | | PADI4, HSPA14, CXCR1, H19, |
| | | | KRTAP19-3, IDDM2, RAC2, YRY1, |
| | | | CLOCK, NGFR, DBH, CHRNA4, |
| | | | CACNA1C, PRKAG2, CHAT, |
| | | | PTGDS, NR1H2, TEK, VEGFB, |
| | | | MEF2C, MAPKAPK2, TNFRSF11A, |
| | | | HSPA9, CYSLTR1, MAT1A, |
| | | | OPRL1, IMPA1, CLCN2, DLD, |
| | | | PSMA6, PSMB8, CHI3L1, |
| | | | ALDH1B1, PARP2, STAR, LBP, |
| | | | ABCC6, RGS2, EFNB2, GJB6, |
| | | | APOA2, AMPD1, DYSF, |
| | | | FDFT1, EMD2, CCR6, GJB3, |
| | | | IL1RL1, ENTPD1, BBS4, CELSR2, |
| | | | F11R, RAPGEF3, HYAL1, ZNF259, |
| | | | ATOX1, ATF6, KHK, SAT1, GGH, |
| | | | TIMP4, SLC4A4, PDE2A, PDE3B, |
| | | | FADS1, FADS2, TMSB4X, TXNIP, |
| | | | LIMS1, RHOB, LY96, FOXO1, |
| | | | PNPLA2, TRH, GJC1, S: C17A5, |
| | | | FTO, GJD2, PRSC1, CASP12, |
| | | | GPBAR1, PXK, IL33, TRIB1, PBX4, |
| | | | NUPR1, 15-SEP, CILP2, TERC, |
| | | | GGT2, MTCO1, UOX, AVP |
| Cataract | eye | | CRYAA, CRYA1, CRYBB2, |
| | | | CRYB2, PITX3, BFSP2, CP49, |
| | | | CP47, CRYAA, CRYA1, PAX6, |
| | | | AN2, MGDA, CRYBA1, CRYB1, |
| | | | CRYGC, CRYG3, CCL, LIM2, |
| | | | MP19, CRYGD, CRYG4, BFSP2, |
| | | | CP49, CP47, HSF4, CTM, HSF4, |
| | | | CTM, MIP, AQP0, CRYAB, |
| | | | CRYA2, CTPP2, CRYBB1, CRYGD, |
| | | | CRYG4, CRYBB2, CRYB2, |
| | | | CRYGC, CRYG3, CCL, CRYAA, |
| | | | CRYA1, GJA8, CX50, CAE1, GJA3, |
| | | | CX46, CZP3, CAE3, CCM1, CAM, |
| | | | KRIT1 |
| CDKL-5 Deficiencies or | Brain, CNS | | CDKL5 |
| Mediated Diseases | | | |
| Charcot-Marie-Tooth (CMT) | Nervous system | Muscles | PMP22 (CMT1A and E), MPZ |
| disease (Types 1, 2, 3, 4,) | | (dystrophy) | (CMT1B), LITAF (CMT1C), EGR2 |
| | | | (CMT1D), NEFL (CMT1F), GJB1 |
| | | | (CMT1X), MFN2 (CMT2A), KIF1B |
| | | | (CMT2A2B), RAB7A (CMT2B), |
| | | | TRPV4 (CMT2C), GARS (CMT2D), |
| | | | NEFL (CMT2E), GAPD1 (CMT2K), |
| | | | HSPB8 (CMT2L), DYNC1H1, |
| | | | CMT2O), LRSAM1 (CMT2P), |
| | | | IGHMBP2 (CMT2S), MORC2 |
| | | | (CMT2Z), GDAP1 (CMT4A), |
| | | | MTMR2 or SBF2/MTMR13 |
| | | | (CMT4B), SH3TC2 (CMT4C), |
| | | | NDRG1 (CMT4D), PRX (CMT4F), |
| | | | FIG. 4 (CMT4J), NT-3 |
| Chédiak-Higashi Syndrome | Immune system | Skin, hair, eyes, | LYST |
| | | neurons | |
| Choroidermia | | | CHM, REP1, |
| Chorioretinal atrophy | eye | | PRDM13, RGR, TEAD1 |
| Chronic Granulomatous Disease | Immune system | | CYBA, CYBB, NCF1, NCF2, NCF4 |
| Chronic Mucocutaneous | Immune system | | AIRE, CARD9, CLEC7A IL12B, |
| Candidiasis | | | IL12B1, IL1F, IL17RA, IL17RC, |
| | | | RORC, STAT1, STAT3, TRAF31P2 |
| Cirrhosis | liver | | KRT18, KRT8, CIRH1A, NAIC, |
| | | | TEX292, KIAA1988 |
| Colon cancer (Familial | Gastrointestinal | | FAP: APC HNPCC: MSH2, |
| adenomatous polyposis (FAP) | | | MLH1, PMS2, SH6, PMS1 |
| and hereditary nonpolyposis | | | |
| colon cancer (HNPCC)) | | | |
| Combined Immunodeficiency | Immune System | | IL2RG, SCIDX1, SCIDX, IMD4); |
| | | | HIV-1 (CCL5, SCYA5, D17S136E, |
| | | | TCP228 |
| Cone(-rod) dystrophy | eye | | AIPL1, CRX, GUA1A, GUCY2D, |
| | | | PITPM3, PROM1, PRPH2, RIMS1, |
| | | | SEMA4A, ABCA4, ADAM9, ATF6, |
| | | | C21ORF2, C8ORF37, CACNA2D4, |
| | | | CDHR1, CERKL, CNGA3, CNGB3, |
| | | | CNNM4, CNAT2, IFT81, KCNV2, |
| | | | PDE6C, PDE6H, POC1B, RAX2, |
| | | | RDH5, RPGRIP1, TTLL5, RetCG1, |
| | | | GUCY2E |
| Congenital Stationary Night | eye | | CABP4, CACNA1F, CACNA2D4, |
| Blindness | | | GNAT1, CPR179, GRK1, GRM6, |
| | | | LRIT3, NYX, PDE6B, RDH5, RHO, |
| | | | RLBP1, RPE65, SAG, SLC24A1, |
| | | | TRPM1, |
| Congenital Fructose Intolerance | Metabolism | | ALDOB |
| Cori's Disease (Glycogen | Various- | | AGL |
| Storage Disease Type III) | wherever | | |
| | glycogen | | |
| | accumulates, | | |
| | particularly | | |
| | liver, heart, | | |
| | skeletal muscle | | |
| Corneal clouding and dystrophy | eye | | APOA1, TGFBI, CSD2, CDGG1, |
| | | | CSD, BIGH3, CDG2, TACSTD2, |
| | | | TROP2, M1S1, VSX1, RINX, PPCD, |
| | | | PPD, KTCN, COL8A2, FECD, |
| | | | PPCD2, PIP5K3, CFD |
| Cornea plana congenital | | | KERA, CNA2 |
| Cri du chat Syndrome, also | | | Deletions involving only band 5p15.2 |
| known as 5p syndrome and cat | | | to the entire short arm of chromosome |
| cry syndrome | | | 5, e.g. CTNND2, TERT, |
| Cystic Fibrosis (CF) | Lungs and | Pancreas, liver, | CTFR, ABCC7, CF, MRP7, |
| | respiratory | digestive system, | SCNN1A, those described in |
| | system | reproductive | WO2015157070 |
| | | system, exocrine, | |
| | | glands, | |
| Diabetic nephropathy | kidney | | Gremlin, 12/15- lipoxygenase, |
| | | | TIM44, |
| Dent Disease (Types 1 and 2) | Kidney | | Type 1: CLCN5, Type 2: ORCL |
| Dentatorubro-Pallidoluysian | CNS, brain, | | Atrophin-1 and Atn1 |
| Atrophy (DRPLA) (aka Haw | muscle | | |
| River and Naito-Oyanagi | | | |
| Disease) | | | |
| Down Syndrome | various | | Chromosome 21 trisomy |
| Drug Addiction | Brain | | Prkce; Drd2; Drd4; ABAT; |
| | | | GRIA2; Grm5; Grin1; Htr1b; Grin2a; |
| | | | Drd3; Pdyn; Grial |
| Duane syndrome (Types 1, 2, | eye | | CHN1, indels on chromosomes 4 and |
| and 3, including subgroups A, B | | | 8 |
| and C). Other names for this | | | |
| condition include: Duane's | | | |
| Retraction Syndrome (or DR | | | |
| syndrome), Eye Retraction | | | |
| Syndrome, Retraction | | | |
| Syndrome, Congenital retraction | | | |
| syndrome and Stilling-Turk- | | | |
| Duane Syndrome | | | |
| Duchenne muscular dystrophy | muscle | Cardiovascular, | DMD, BMD, dystrophin gene, intron |
| (DMD) | | respiratory | flanking exon 51 of DMD gene, exon |
| | | | 51 mutations in DMD gene, see also |
| | | | WO2013163628 and US Pat. Pub. |
| | | | 20130145487 |
| Edward's Syndrome | | | Complete or partial trisomy of |
| (Trisomy 18) | | | chromosome 18 |
| Ehlers-Danlos Syndrome (Types | Various | | COL5A1, COL5A2, COL1A1, |
| I-VI) | depending on | | COL3A1, TNXB, PLOD1, COL1A2, |
| | type: including | | FKBP14 and ADAMTS2 |
| | musculoskeletal, | | |
| | eye, vasculature, | | |
| | immune, and | | |
| | skin | | |
| Emery-Dreifuss muscular | muscle | | LMNA, LMN1, EMD2, FPLD, |
| dystrophy | | | CMD1A, HGPS, LGMD1B, LMNA, |
| | | | LMN1, EMD2, FPLD, CMD1A |
| Enhanced S-Cone Syndrome | eye | | NR2E3, NRL |
| Fabry's Disease | Various - | | GLA |
| | including skin, | | |
| | eyes, and | | |
| | gastrointestinal | | |
| | system, kidney, | | |
| | heart, brain, | | |
| | nervous system | | |
| Facioscapulohumeral muscular | muscles | | FSHMD1A, FSHD1A, FRG1, |
| dystrophy | | | |
| Factor H and Factor H-like 1 | blood | | HF1, CFH, HUS |
| Factor V Leiden thrombophilia | blood | | Factor V (F5) |
| and Factor V deficiency | | | |
| Factor V and Factor VII | blood | | MCFD2 |
| deficiency | | | |
| Factor VII deficiency | blood | | F7 |
| Factor X deficiency | blood | | F10 |
| Factor XI deficiency | blood | | F11 |
| Factor XII deficiency | blood | | F12, HAF |
| Factor XIIIA deficiency | blood | | F13A1, F13A |
| Factor XIIIB deficiency | blood | | F13B |
| Familial Hypercholestereolemia | Cardiovascular | | APOB, LDLR, PCSK9 |
| | system | | |
| Familial Mediterranean Fever | Various- | Heart, kidney, | MEFV |
| (FMF) also called recurrent | organs/tissues | brain/CNS, | |
| polyserositis or familial | with serous or | reproductive | |
| paroxysmal polyserositis | synovial | organs | |
| | membranes, | | |
| | skin, joints | | |
| Fanconi Anemia | Various - blood | | FANCA, FACA, FA1, FA, FAA, |
| | (anemia), | | FAAP95, FAAP90, FLJ34064, |
| | immune system, | | FANCC, FANCG, RAD51, BRCA1, |
| | cognitive, | | BRCA2, BRIP1, BACH1, FANCJ, |
| | kidneys, eyes, | | FANCB, FANCD1, FANCD2, |
| | musculoskeletal | | FANCD, FAD, FANCE, FACE, |
| | | | FANCF, FANCI, ERCC4, FANCL, |
| | | | FANCM, PALB2, RAD51C, SLX4, |
| | | | UBE2T, FANCB, XRCC9, PHF9, |
| | | | KIAA1596 |
| Fanconi Syndrome Types I | kidneys | | FRTS1, GATM |
| (Childhood onset) and II (Adult | | | |
| Onset) | | | |
| Fragile X syndrome and related | brain | | FMR1, FMR2; FXR1; FXR2; |
| disorders | | | mGLUR5 |
| Fragile XE Mental Retardation | Brain, nervous | | FMR1 |
| (aka Martin Bell syndrome) | system | | |
| Friedreich Ataxia (FRDA) | Brain, nervous | heart | FXN/X25 |
| | system | | |
| Fuchs endothelial corneal | Eye | | TCF4; COL8A2 |
| dystrophy | | | |
| Galactosemia | Carbohydrate | Various-where | GALT, GALK1, and GALE |
| | metabolism | galactose | |
| | disorder | accumulates - | |
| | | liver, brain, eyes | |
| Gastrointestinal Epithelial | | | CISH |
| Cancer, GI cancer | | | |
| Gaucher Disease (Types 1, 2, | Fat metabolism | Various-liver, | GBA |
| and 3, as well as other unusual | disorder | spleen, blood, | |
| forms that may not fit into these | | CNS, skeletal | |
| types) | | system | |
| Griscelli syndrome | | | |
| Glaucoma | eye | | MYOC, TIGR, GLC1A, JOAG, |
| | | | GPOA, OPTN, GLC1E, FIP2, HYPL, |
| | | | NRP, CYP1B1, GLC3A, OPA1, |
| | | | NTG, NPG, CYP1B1, GLC3A, those |
| | | | described in WO2015153780 |
| Glomerulo sclerosis | kidney | | CC chemokine ligand 2 |
| Glycogen Storage Diseases | Metabolism | | SLC2A2, GLUT2, G6PC, G6PT, |
| Types I-VI -See also Cori's | Diseases | | G6PT1, GAA, LAMP2, LAMPB, |
| Disease, Pompe's Disease, | | | AGL, GDE, GBE1, GYS2, PYGL, |
| McArdle's disease, Hers | | | PFKM, see also Cori's Disease, |
| Disease, and Von Gierke's | | | Pompe's Disease, McArdle's disease, |
| disease | | | Hers Disease, and Von Gierke's |
| | | | disease |
| RBC Glycolytic enzyme | blood | | any mutations in a gene for an |
| deficiency | | | enzyme in the glycolysis pathway |
| | | | including mutations in genes for |
| | | | hexokinases I and II, glucokinase, |
| | | | phosphoglucose isomerase, |
| | | | phosphofructokinase, aldolase Bm |
| | | | triosephosphate isomerease, |
| | | | glyceraldehydee-3-phosphate |
| | | | dehydrogenase, |
| | | | phosphoglycerokinase, |
| | | | phosphoglycerate mutase, enolase I, |
| | | | pyruvate kinase |
| Hartnup's disease | Malabsorption | Various- brain, | SLC6A19 |
| | disease | gastrointestinal, | |
| | | skin, | |
| Hearing Loss | ear | | NOX3, Hes5, BDNF, |
| Hemochromatosis (HH) | Iron absorption | Various-wherever | HFE and H63D |
| | regulation | iron accumulates, | |
| | disease | liver, heart, | |
| | | pancreas, joints, | |
| | | pituitary gland | |
| Hemophagocytic | blood | | PRF1, HPLH2, UNC13D, MUNC13- |
| lymphohistiocytosis disorders | | | 4, HPLH3, HLH3, FHL3 |
| Hemorrhagic disorders | blood | | PI, ATT, F5 |
| Hers disease (Glycogen storage | liver | muscle | PYGL |
| disease Type VI) | | | |
| Hereditary angioedema (HAE) | | | kalikrein B1 |
| Hereditary Hemorrhagic | Skin and | | ACVRL1, ENG and SMAD4 |
| Telangiectasia (Osler-Weber- | mucous | | |
| Rendu Syndrome) | membranes | | |
| Hereditary Spherocytosis | blood | | NK1, EPB42, SLC4A1, SPTA1, and |
| | | | SPTB |
| Hereditary Persistence of Fetal | blood | | HBG1, HBG2, BCL11A, promoter |
| Hemoglobin | | | region of HBG 1 and/or 2 (in the |
| | | | CCAAT box) |
| Hemophilia (hemophilia A | blood | | A: FVIII, F8C, HEMA |
| (Classic) a B (aka Christmas | | | B: FVIX, HEMB |
| disease) and C) | | | C: F9, F11 |
| Hepatic adenoma | liver | | TCF1, HNF1A, MODY3 |
| Hepatic failure, early onset, and | liver | | SCOD1, SCO1 |
| neurologic disorder | | | |
| Hepatic lipase deficiency | liver | | LIPC |
| Hepatoblastoma, cancer and | liver | | CTNNB1, PDGFRL, PDGRL, |
| carcinomas | | | PRLTS, AXIN1, AXIN, CTNNB1, |
| | | | TP53, P53, LFS1, IGF2R, MPRI, |
| | | | MET, CASP8, MCH5 |
| Hermansky-Pudlak syndrome | Skin, eyes, | | HPS1, HPS3, HPS4, HPS5, HPS6, |
| | blood, lung, | | HPS7, DTNBP1, BLOC1, BLOC1S2, |
| | kidneys, | | BLOC3 |
| | intestine | | |
| HIV susceptibility or infection | Immune system | | IL10, CSIF, CMKBR2, CCR2, |
| | | | CMKBR5, CCCKR5 (CCR5), those |
| | | | in WO2015148670A1 |
| Holoprosencephaly (HPE) | brain | | ACVRL1, ENG, SMAD4 |
| (Alobar, Semilobar, and Lobar) | | | |
| Homocystinuria | Metabolic | Various- | CBS, MTHFR, MTR, MTRR, and |
| | disease | connective tissue, | MMADHC |
| | | muscles, CNS, | |
| | | cardiovascular | |
| | | system | |
| HPV | | | HPV16 and HPV18 E6/E7 |
| HSV1, HSV2, and related | eye | | HSV1 genes (immediate early and |
| keratitis | | | late HSV-1 genes (UL1, 1.5, 5, 6, 8, |
| | | | 9, 12, 15, 16, 18, 19, 22, 23, 26, 26.5, |
| | | | 27, 28, 29, 30, 31, 32, 33, 34, 35, 36, |
| | | | 37, 38, 42, 48, 49.5, 50, 52, 54, S6, |
| | | | RL2, RS1, those described in |
| | | | WO2015153789, WO2015153791 |
| Hunter's Syndrome (aka | Lysosomal | Various- liver, | IDS |
| Mucopolysaccharidosis type II) | storage disease | spleen, eye, joint, | |
| | | heart, brain, | |
| | | skeletal | |
| Huntington's disease (HD) and | Brain, nervous | | HD, HTT, IT15, PRNP, PRIP, JPH3, |
| HD-like disorders | system | | JP3, HDL2, TBP, SCA17, PRKCE; |
| | | | IGF1; EP300; RCOR1; PRKCZ; |
| | | | HDAC4; and TGM2, and those |
| | | | described in WO2013130824, |
| | | | WO2015089354 |
| Hurler's Syndrome (aka | Lysosomal | Various- liver, | IDUA, α-L-iduronidase |
| mucopolysaccharidosis type I H, | storage disease | spleen, eye, joint, | |
| MPS IH) | | heart, brain, | |
| | | skeletal | |
| Hurler-Scheie syndrome (aka | Lysosomal | Various- liver, | IDUA, α-L-iduronidase |
| mucopolysaccharidosis type I H- | storage disease | spleen, eye, joint, | |
| S, MPS I H-S) | | heart, brain, | |
| | | skeletal | |
| hyaluronidase deficiency (aka | Soft and | | HYAL1 |
| MPS IX) | connective | | |
| | tissues | | |
| Hyper IgM syndrome | Immune system | | CD40L |
| Hyper- tension caused renal | kidney | | Mineral corticoid receptor |
| damage | | | |
| Immunodeficiencies | Immune System | | CD3E, CD3G, AICDA, AID, |
| | | | HIGM2, TNFRSF5, CD40, UNG, |
| | | | DGU, HIGM4, TNFSF5, CD40LG, |
| | | | HIGM1, IGM, FOXP3, IPEX, AIID, |
| | | | XPID, PIDX, TNFRSF14B, TACI |
| Inborn errors of metabolism: | Metabolism | Various organs | See also: Carbohydrate metabolism |
| including urea cycle disorders, | diseases, liver | and cells | disorders (e.g. galactosemia), Amino |
| organic acidemias), fatty acid | | | acid Metabolism disorders (e.g. |
| oxidation defects, amino | | | phenylketonuria), Fatty acid |
| acidopathies, carbohydrate | | | metabolism (e.g. MCAD deficiency), |
| disorders, mitochondrial | | | Urea Cycle disorders (e.g. |
| disorders | | | Citrullinemia), Organic acidemias |
| | | | (e.g. Maple Syrup Urine disease), |
| | | | Mitochondrial disorders (e.g. |
| | | | MELAS), peroxisomal disorders (e.g. |
| | | | Zellweger syndrome) |
| Inflammation | Various | | IL-10; IL-1 (IL-la; IL-1b); IL-13; IL- |
| | | | 17 (IL-17a (CTLA8); IL- |
| | | | 17b; IL-17c; IL-17d; IL-17f); II-23; |
| | | | Cx3cr1; ptpn22; TNFa; |
| | | | NOD2/CARD15 for IBD; IL-6; IL-12 |
| | | | (IL-12a; IL-12b); |
| | | | CTLA4; Cx3cl1 |
| Inflammatory Bowel Diseases | Gastrointestinal | Joints, skin | NOD2, IRGM, LRRK2, ATG5, |
| (e.g. Ulcerative Colitis and | | | ATG16L1, IRGM, GATM, ECM1, |
| Chron's Disease) | | | CDH1, LAMB1, HNF4A, GNA12, |
| | | | IL10, CARD9/15. CCR6, IL2RA, |
| | | | MST1, TNFSF15, REL, STAT3, |
| | | | IL23R, IL12B, FUT2 |
| Interstitial renal fibrosis | kidney | | TGF-β type II receptor |
| Job's Syndrome (aka Hyper IgE | Immune System | | STAT3, DOCK8 |
| Syndrome) | | | |
| Juvenile Retinoschisis | eye | | RS1, XLRS1 |
| Kabuki Syndrome 1 | | | MLL4, KMT2D |
| Kennedy Disease (aka | Muscles, brain, | | SBMA/SMAX1/AR |
| Spinobulbar Muscular Atrophy) | nervous system | | |
| Klinefelter syndrome | Various- | | Extra X chromosome in males |
| | particularly | | |
| | those involved | | |
| | in development | | |
| | of male | | |
| | characteristics | | |
| Lafora Disease | Brain, CNS | | EMP2A and EMP2B |
| Leber Congenital Amaurosis | eye | | CRB1, RP12, CORD2, CRD, CRX, |
| | | | IMPDH1, OTX2, AIPL1, CABP4, |
| | | | CCT2, CEP290, CLUAP1, CRB1, |
| | | | CRX, DTHD1, GDF6, GUCY2D, |
| | | | IFT140, IQCB1, KCNJ13, LCA5, |
| | | | LRAT, NMNAT1, PRPH2, RD3, |
| | | | RDH12, RPE65, RP20, RPGRIP1, |
| | | | SPATA7, TULP1, LCA1, LCA4, |
| | | | GUC2D, CORD6, LCA3, |
| Lesch-Nyhan Syndrome | Metabolism | Various - joints, | HPRT1 |
| | disease | cognitive, brain, | |
| | | nervous system | |
| Leukocyte deficiencies and | blood | | ITGB2, CD18, LCAMB, LAD, |
| disorders | | | EIF2B1, EIF2BA, EIF2B2, EIF2B3, |
| | | | EIF2B5, LVWM, CACH, CLE, |
| | | | EIF2B4 |
| Leukemia | Blood | | TAL1, TCL5, SCL, TAL2, FLT3, |
| | | | NBS1, NBS, ZNFN1A1, IK1, LYF1, |
| | | | HOXD4, HOX4B, BCR, CML, PHL, |
| | | | ALL, ARNT, KRAS2, RASK2, |
| | | | GMPS, AF10, ARHGEF12, LARG, |
| | | | KIAA0382, CALM, CLTH, CEBPA, |
| | | | CEBP, CHIC2, BTL, FLT3, KIT, |
| | | | PBT, LPP, NPM1, NUP214, D9S46E, |
| | | | CAN, CAIN, RUNX1, CBFA2, |
| | | | AML1, WHSC1L1, NSD3, FLT3, |
| | | | AF1Q, NPM1, NUMA1, ZNF145, |
| | | | PLZF, PML, MYL, STAT5B, AF10, |
| | | | CALM, CLTH, ARL11, ARLTS1, |
| | | | P2RX7, P2X7, BCR, CML, PHL, |
| | | | ALL, GRAF, NF1, VRNF, WSS, |
| | | | NFNS, PTPN11, PTP2C, SHP2, NS1, |
| | | | BCL2, CCND1, PRAD1, BCL1, |
| | | | TCRA, GATA1, GF1, ERYF1, |
| | | | NFE1, ABL1, NQO1, DIA4, |
| | | | NMOR1, NUP214, D9S46E, CAN, |
| | | | CAIN |
| Limb-girdle muscular dystrophy | muscle | | LGMD |
| diseases | | | |
| Lowe syndrome | brain, eyes, | | OCRL |
| | kidneys | | |
| Lupus glomerulo- nephritis | kidney | | MAPK1 |
| Machado- | Brain, CNS, | | ATX3 |
| Joseph's Disease (also known as | muscle | | |
| Spinocerebellar ataxia Type 3) | | | |
| Macular degeneration | eye | | ABC4, CBC1, CHM1, APOE, |
| | | | C1QTNF5, C2, C3, CCL2, CCR2, |
| | | | CD36, CFB, CFH, CFHR1, CFHR3, |
| | | | CNGB3, CP, CRP, CST3, CTSD, |
| | | | CX3CR1, ELOVL4, ERCC6, |
| | | | FBLN5, FBLN6, FSCN2, HMCN1, |
| | | | HTRA1, IL6, IL8, PLEKHA1, |
| | | | PROM1, PRPH2, RPGR, |
| | | | SERPING1, TCOF1, TIMP3, TLR3 |
| Macular Dystrophy | eye | | BEST1, C1QTNF5, CTNNA1, |
| | | | EFEMP1, ELOVL4, FSCN2, |
| | | | GUCA1B, HMCN1, IMPG1, OTX2, |
| | | | PRDM13, PROM1, PRPH2, RP1L1, |
| | | | TIMP3, ABCA4, CFH, DRAM2, |
| | | | IMG1, MFSD8, ADMD, STGD2, |
| | | | STGD3, RDS, RP7, PRPH, AVMD, |
| | | | AOFMD, VMD2 |
| Malattia Leventinesse | eye | | EFEMP1, FBLN3 |
| Maple Syrup Urine Disease | Metabolism | | BCKDHA, BCKDHB, and DBT |
| | disease | | |
| Marfan syndrome | Connective | Musculoskeletal | FBN1 |
| | tissue | | |
| Maroteaux-Lamy Syndrome | Musculoskeletal | Liver, spleen | ARSB |
| (aka MPS VI) | system, nervous | | |
| | system | | |
| McArdle's Disease (Glycogen | Glycogen | muscle | PYGM |
| Storage Disease Type V) | storage disease | | |
| Medullary cystic kidney disease | kidney | | UMOD, HNFJ, FJHN, MCKD2, |
| | | | ADMCKD2 |
| Metachromatic leukodystrophy | Lysosomal | Nervous system | ARSA |
| | storage disease | | |
| Methylmalonic acidemia | Metabolism | | MMAA, MMAB, MUT, MMACHC, |
| (MMA) | disease | | MMADHC, LMBRD1 |
| Morquio Syndrome (aka MPS | Connective | heart | GALNS |
| IV A and B) | tissue, skin, | | |
| | bone, eyes | | |
| Mucopolysaccharidosis diseases | Lysosomal | | See also Hurler/Scheie syndrome, |
| (Types I H/S, I H, II, III A B and | storage disease - | | Hurler disease, Sanfillipo syndrome, |
| C, I S, IVA and B, IX, VII, and | affects various | | Scheie syndrome, Morquio syndrome, |
| VI) | organs/tissues | | hyaluronidase deficiency, Sly |
| | | | syndrome, and Maroteaux-Lamy |
| | | | syndrome |
| Muscular Atrophy | muscle | | VAPB, VAPC, ALS8, SMN1, SMA1, |
| | | | SMA2, SMA3, SMA4, BSCL2, |
| | | | SPG17, GARS, SMAD1, CMT2D, |
| | | | HEXB, IGHMBP2, SMUBP2, |
| | | | CATF1, SMARD1 |
| Muscular dystrophy | muscle | | FKRP, MDC1C, LGMD2I, LAMA2, |
| | | | LAMM, LARGE, KIAA0609, |
| | | | MDC1D, FCMD, TTID, MYOT, |
| | | | CAPN3, CANP3, DYSF, LGMD2B, |
| | | | SGCG, LGMD2C, DMDA1, SCG3, |
| | | | SGCA, ADL, DAG2, LGMD2D, |
| | | | DMDA2, SGCB, LGMD2E, SGCD, |
| | | | SGD, LGMD2F, CMD IL, TCAP, |
| | | | LGMD2G, CMD1N, TRIM32, |
| | | | HT2A, LGMD2H, FKRP, MDC1C, |
| | | | LGMD2I, TTN, CMD1G, TMD, |
| | | | LGMD2J, POMT1, CAV3, |
| | | | LGMD1C, SEPN1, SELN, RSMD1, |
| | | | PLEC1, PLTN, EBS1 |
| Myotonic dystrophy (Type 1 and | Muscles | Eyes, heart, | CNBP (Type 2) and DMPK (Type 1) |
| Type 2) | | endocrine | |
| Neoplasia | | | PTEN; ATM; ATR; EGFR; ERBB2; |
| | | | ERBB3; ERBB4; |
| | | | Notch1; Notch2; Notch3; Notch4; |
| | | | AKT; AKT2; AKT3; HIF; |
| | | | HIF1a; HIF3a; Met; HRG; Bcl2; |
| | | | PPAR alpha; PPAR |
| | | | gamma; WT1 (Wilms Tumor); FGF |
| | | | Receptor Family |
| | | | members (5 members: 1, 2, 3, 4, 5); |
| | | | CDKN2a; APC; RB |
| | | | (retinoblastoma); MEN1; VHL; |
| | | | BRCA1; BRCA2; AR |
| | | | (Androgen Receptor); TSG101; IGF; |
| | | | IGF Receptor; Igf1 (4 |
| | | | variants); Igf2 (3 variants); Igf 1 |
| | | | Receptor; Igf 2 Receptor; |
| | | | Bax; Bcl2; caspases family (9 |
| | | | members: |
| | | | 1, 2, 3, 4, 6, 7, 8, 9, 12); Kras; Apc |
| Neurofibromatosis (NF) (NF1, | brain, spinal | | NF1, NF2 |
| formerly Recklinghausen's NF, | cord, nerves, | | |
| and NF2) | and skin | | |
| Niemann-Pick Lipidosis (Types | Lysosomal | Various- where | Types A and B: SMPD1; Type C: |
| A, B, and C) | Storage Disease | sphingomyelin | NPC1 or NPC2 |
| | | accumulates, | |
| | | particularly | |
| | | spleen, liver, | |
| | | blood, CNS | |
| Noonan Syndrome | Various - | | PTPN11, SOS1, RAF1 and KRAS |
| | musculoskeletal, | | |
| | heart, eyes, | | |
| | reproductive | | |
| | organs, blood | | |
| Norrie Disease or X-linked | eye | | NDP |
| Familial Exudative | | | |
| Vitreoretinopathy | | | |
| North Carolina Macular | eye | | MCDR1 |
| Dystrophy | | | |
| Osteogenesis imperfecta (OI) | bones, | | COL1A1, COL1A2, CRTAP, P3H |
| (Types I, II, III, IV, V, VI, VII) | musculoskeletal | | |
| Osteopetrosis | bones | | LRP5, BMND1, LRP7, LR3, OPPG, |
| | | | VBCH2, CLCN7, CLC7, OPTA2, |
| | | | OSTM1, GL, TCIRG1, TIRC7, |
| | | | OC116, OPTB1 |
| Patau's Syndrome | Brain, heart, | | Additional copy of chromosome 13 |
| (Trisomy 13) | skeletal system | | |
| Parkinson's disease (PD) | Brain, nervous | | SNCA (PARK1), UCHL1 (PARK 5), |
| | system | | and LRRK2 (PARK8), (PARK3), |
| | | | PARK2, PARK4, PARK7 (PARK7), |
| | | | PINK1 (PARK6); x-Synuclein, DJ-1, |
| | | | Parkin, NR4A2, NURR1, NOT, |
| | | | TINUR, SNCAIP, TBP, SCA17, |
| | | | NCAP, PRKN, PDJ, DBH, NDUFV2 |
| Pattern Dystrophy of the RPE | eye | | RDS/peripherin |
| Phenylketonuria (PKU) | Metabolism | Various due to | PAH, PKU1, QDPR, DHPR, PTS |
| | disorder | build-up of | |
| | | phenylalanine, | |
| | | phenyl ketones in | |
| | | tissues and CNS | |
| Polycystic kidney and hepatic | Kidney, liver | | FCYT, PKHD1, ARPKD, PKD1, |
| disease | | | PKD2, PKD4, PKDTS, PRKCSH, |
| | | | G19P1, PCLD, SEC63 |
| Pompe's Disease | Glycogen | Various - heart, | GAA |
| | storage disease | liver, spleen | |
| Porphyria (actually refers to a | Various- | | ALAD, ALAS2, CPOX, FECH, |
| group of different diseases all | wherever heme | | HMBS, PPOX, UROD, or UROS |
| having a specific heme | precursors | | |
| production process abnormality) | accumulate | | |
| posterior polymorphous corneal | eyes | | TCF4; COL8A2 |
| dystrophy | | | |
| Primary Hyperoxaluria (e.g. type | Various - eyes, | | LDHA (lactate dehydrogenase A) and |
| 1) | heart, kidneys, | | hydroxyacid oxidase 1 (HAO1) |
| | skeletal system | | |
| Primary Open Angle Glaucoma | eyes | | MYOC |
| (POAG) | | | |
| Primary sclerosing cholangitis | Liver, | | TCF4; COL8A2 |
| | gallbladder | | |
| Progeria (also called | All | | LMNA |
| Hutchinson-Gilford progeria | | | |
| syndrome) | | | |
| Prader-Willi Syndrome | Musculoskeletal | | Deletion of region of short arm of |
| | system, brain, | | chromosome 15, including UBE3A |
| | reproductive | | |
| | and endocrine | | |
| | system | | |
| Prostate Cancer | prostate | | HOXB13, MSMB, GPRC6A, TP53 |
| Pyruvate Dehydrogenase | Brain, nervous | | PDHA1 |
| Deficiency | system | | |
| Kidney/Renal carcinoma | kidney | | RLIP76, VEGF |
| Rett Syndrome | Brain | | MECP2, RTT, PPMX, MRX16, |
| | | | MRX79, CDKL5, STK9, MECP2, |
| | | | RTT, PPMX, MRX16, MRX79, x- |
| | | | Synuclein, DJ-1 |
| Retinitis pigmentosa (RP) | eye | | ADIPOR1, ABCA4, AGBL5, |
| | | | ARHGEF18, ARL2BP, ARL3, |
| | | | ARL6, BEST1, BBS1, BBS2, |
| | | | C2ORF71, C8ORF37, CA4, CERKL, |
| | | | CLRN1, CNGA1, CMGB1, CRB1, |
| | | | CRX, CYP4V2, DHDDS, DHX38, |
| | | | EMC1, EYS, FAM161A, FSCN2, |
| | | | GPR125, GUCA1B, HK1, HPRPF3, |
| | | | HGSNAT, IDH3B, IMPDH1, |
| | | | IMPG2, IFT140, IFT172, KLHL7, |
| | | | KIAA1549, KIZ, LRAT, MAK, |
| | | | MERTK, MVK, NEK2, NUROD1, |
| | | | NR2E3, NRL, OFD1, PDE6A, |
| | | | PDE6B, PDE6G, POMGNT1, PRCD, |
| | | | PROM1, PRPF3, PRPF4, PRPF6, |
| | | | PRPF8, PRPF31, PRPH2, RPB3, |
| | | | RDH12, REEP6, RP39, RGR, RHO, |
| | | | RLBP1, ROM1, RP1, RP1L1, RPY, |
| | | | RP2, RP9, RPE65, RPGR, SAMD11, |
| | | | SAG, SEMA4A, SLC7A14, |
| | | | SNRNP200, SPP2, SPATA7, |
| | | | TRNT1, TOPORS, TTC8, TULP1, |
| | | | USH2A, ZFN408, ZNF513, see also |
| | | | 20120204282 |
| Scheie syndrome (also known as | Various- liver, | | IDUA, α-L-iduronidase |
| mucopolysaccharidosis type I | spleen, eye, | | |
| S(MPS I-S)) | joint, heart, | | |
| | brain, skeletal | | |
| Schizophrenia | Brain | | Neuregulin1 (Nrg1); Erb4 (receptor |
| | | | for Neuregulin); |
| | | | Complexin1 (Cplx1); Tph1 |
| | | | Tryptophan hydroxylase; Tph2 |
| | | | Tryptophan hydroxylase 2; Neurexin |
| | | | 1; GSK3; GSK3a; |
| | | | GSK3b; 5-HTT (Slc6a4); COMT; |
| | | | DRD (Drd1a); SLC6A3; DAOA; |
| | | | DTNBP1; Dao (Dao1); TCF4; |
| | | | COL8A2 |
| Secretase Related Disorders | Various | | APH-1 (alpha and beta); PSEN1; |
| | | | NCSTN; PEN-2; Nos1, Parp1, Nat1, |
| | | | Nat2, CTSB, APP, APH1B, PSEN2, |
| | | | PSENEN, BACE1, ITM2B, CTSD, |
| | | | NOTCH1, TNF, INS, DYT10, |
| | | | ADAM17, APOE, ACE, STN, TP53, |
| | | | IL6, NGFR, IL1B, ACHE, CTNNB1, |
| | | | IGF1, IFNG, NRG1, CASP3, |
| | | | MAPK1, CDH1, APBB1, HMGCR, |
| | | | CREB1, PTGS2, HES1, CAT, |
| | | | TGFB1, ENO2, ERBB4, TRAPPC10, |
| | | | MAOB, NGF, MMP12, JAG1, |
| | | | CD40LG, PPARG, FGF2, LRP1, |
| | | | NOTCH4, MAPK8, PREP, |
| | | | NOTCH3, PRNP, CTSG, EGF, REN, |
| | | | CD44, SELP, GHR, ADCYAP1, |
| | | | INSR, GFAP, MMP3, MAPK10, |
| | | | SP1, MYC, CTSE, PPARA, JUN, |
| | | | TIMP1, IL5, IL1A, MMP9, HTR4, |
| | | | HSPG2, KRAS, CYCS, SMG1, |
| | | | IL1R1, PROK1, MAPK3, NTRK1, |
| | | | IL13, MME, TKT, CXCR2, CHRM1, |
| | | | ATXN1, PAWR, NOTCJ2, M6PR, |
| | | | CYP46A1, CSNK1D, MAPK14, |
| | | | PRG2, PRKCA, L1 CAM, CD40, |
| | | | NR112, JAG2, CTNND1, CMA1, |
| | | | SORT1, DLK1, THEM4, JUP, CD46, |
| | | | CCL11, CAV3, RNASE3, HSPA8, |
| | | | CASP9, CYP3A4, CCR3, TFAP2A, |
| | | | SCP2, CDK4, JOF1A, TCF7L2, |
| | | | B3GALTL, MDM2, RELA, CASP7, |
| | | | IDE, FANP4, CASK, ADCYAP1R1, |
| | | | ATF4, PDGFA, C21ORF33, SCG5, |
| | | | RMF123, NKFB1, ERBB2, CAV1, |
| | | | MMP7, TGFA, RXRA, STX1A, |
| | | | PSMC4, P2RY2, TNFRSF21, DLG1, |
| | | | NUMBL, SPN, PLSCR1, UBQLN2, |
| | | | UBQLN1, PCSK7, SPON1, SILV, |
| | | | QPCT, HESS, GCC1 |
| Selective IgA Deficiency | Immune system | | Type 1: MSH5; Type 2: TNFRSF13B |
| Severe Combined | Immune system | | JAK3, JAKL, DCLRE1C, ARTEMIS, |
| Immunodeficiency (SCID) and | | | SCIDA, RAG1, RAG2, ADA, |
| SCID-X1, and ADA-SCID | | | PTPRC, CD45, LCA, IL7R, CD3D, |
| | | | T3D, IL2RG, SCIDX1, SCIDX, |
| | | | IMD4, those identified in US Pat. |
| | | | App. Pub. 20110225664, |
| | | | 20110091441, 20100229252, |
| | | | 20090271881 and 20090222937; |
| Sickle cell disease | blood | | HBB, BCL11A, BCL11Ae, cis- |
| | | | regulatory elements of the B-globin |
| | | | locus, HBG 1/2 promoter, HBG distal |
| | | | CCAAT box region between −92 |
| | | | and −130 of the HBG Transcription Start |
| | | | Site, those described in |
| | | | WO2015148863, WO 2013/126794, |
| | | | US Pat. Pub. 20110182867 |
| Sly Syndrome (aka MPS VII) | | | GUSB |
| Spinocerebellar Ataxias (SCA | | | ATXN1, ATXN2, ATX3 |
| types 1, 2, 3, 6, 7, 8, 12 and 17) | | | |
| Sorsby Fundus Dystrophy | eye | | TIMP3 |
| Stargardt disease | eye | | ABCR, ELOVL4, ABCA4, PROM1 |
| Tay-Sachs Disease | Lysosomal | Various - CNS, | HEX-A |
| | Storage disease | brain, eye | |
| Thalassemia (Alpha, Beta, | blood | | HBA1, HBA2 (Alpha), HBB (Beta), |
| Delta) | | | HBB and HBD (delta), LCRB, |
| | | | BCL11A, BCL11Ae, cis-regulatory |
| | | | elements of the B-globin locus, HBG |
| | | | 1/2 promoter, those described in |
| | | | WO2015148860, US Pat. Pub. |
| | | | 20110182867, 2015/148860 |
| Thymic Aplasia (DiGeorge | Immune system, | | deletion of 30 to 40 genes in the |
| Syndrome; 22q11.2 deletion | thymus | | middle of chromosome 22 at |
| syndrome) | | | a location known as 22q11.2, |
| | | | including TBX1, DGCR8 |
| Transthyretin amyloidosis | liver | | TTR (transthyretin) |
| (ATTR) | | | |
| trimethylaminuria | Metabolism | | FMO3 |
| | disease | | |
| Trinucleotide Repeat Disorders | Various | | HTT; SBMA/SMAX1/AR; |
| (generally) | | | FXN/X25 ATX3; |
| | | | ATXN1; ATXN2; |
| | | | DMPK; Atrophin-1 and Atn1 |
| | | | (DRPLA Dx); CBP (Creb-BP - global |
| | | | instability); VLDLR; Atxn7; Atxn10; |
| | | | FEN1, TNRC6A, PABPN1, JPH3, |
| | | | MED15, ATXN1, ATXN3, TBP, |
| | | | CACNA1A, ATXN80S, PPP2R2B, |
| | | | ATXN7, TNRC6B, TNRC6C, |
| | | | CELF3, MAB21L1, MSH2, |
| | | | TMEM185A, SIX5, CNPY3, RAXE, |
| | | | GNB2, RPL14, ATXN8, ISR, TTR, |
| | | | EP400, GIGYF2, OGG1, STC1, |
| | | | CNDP1, C10ORF2, MAML3, DKC1, |
| | | | PAXIP1, CASK, MAPT, SP1, POLG, |
| | | | AFF2, THBS1, TP53, ESR1, |
| | | | CGGBP1, ABT1, KLK3, PRNP, |
| | | | JUN, KCNN3, BAX, FRAXA, |
| | | | KBTBD10, MBNL1, RAD51, |
| | | | NCOA3, ERDA1, TSC1, COMP, |
| | | | GGLC, RRAD, MSH3, DRD2, |
| | | | CD44, CTCF, CCND1, CLSPN, |
| | | | MEF2A, PTPRU, GAPDH, TRIM22, |
| | | | WT1, AHR, GPX1, TPMT, NDP, |
| | | | ARX, TYR, EGR1, UNG, NUMBL, |
| | | | FABP2, EN2, CRYGC, SRP14, |
| | | | CRYGB, PDCD1, HOXA1, |
| | | | ATXN2L, PMS2, GLA, CBL, FTH1, |
| | | | IL12RB2, OTX2, HOXA5, POLG2, |
| | | | DLX2, AHRR, MANF, RMEM158, |
| | | | see also 20110016540 |
| Turner's Syndrome (XO) | Various - | | Monosomy X |
| | reproductive | | |
| | organs, and sex | | |
| | characteristics, | | |
| | vasculature | | |
| Tuberous Sclerosis | CNS, heart, | | TSC1, TSC2 |
| | kidneys | | |
| Usher syndrome (Types I, II, and | Ears, eyes | | ABHD12, CDH23, CIB2, CLRN1, |
| III) | | | DFNB31, GPR98, HARS, MYO7A, |
| | | | PCDH15, USH1C, USH1G, USH2A, |
| | | | USH11A, those described in |
| | | | WO2015134812A1 |
| Velocardiofacial syndrome (aka | Various - | | Many genes are deleted, COM, |
| 22q11.2 deletion syndrome, | skeletal, heart, | | TBX1, and other are associated with |
| DiGeorge syndrome, | kidney, immune | | symptoms |
| conotruncal anomaly face | system, brain | | |
| syndrome (CTAF), autosomal | | | |
| dominant Opitz G/BB syndrome | | | |
| or Cayler cardiofacial syndrome) | | | |
| Von Gierke's Disease (Glycogen | Glycogen | Various - liver, | G6PC and SLC37A4 |
| Storage Disease type I) | Storage disease | kidney | |
| Von Hippel-Lindau Syndrome | Various - cell | CNS, Kidney, | VHL |
| | growth | Eye, visceral | |
| | regulation | organs | |
| | disorder | | |
| Von Willebrand Disease (Types | blood | | VWF |
| I, II and III) | | | |
| Wilson Disease | Various - | Liver, brains, | ATP7B |
| | Copper Storage | eyes, other tissues | |
| | Disease | where copper | |
| | | builds up | |
| Wiskott-Aldrich Syndrome | Immune System | | WAS |
| Xeroderma Pigmentosum | Skin | Nervous system | POLH |
| XXX Syndrome | Endocrine, brain | | X chromosome trisomy |

In one embodiment, the compositions, systems, or components thereof can be used treat or prevent a disease in a subject by modifying one or more genes associated with one or more cellular functions, such as any one or more of those in Table 7. In one embodiment, the disease is a genetic disease or disorder. In some of embodiments, the composition, system, or component thereof can modify one or more genes or polynucleotides associated with one or more genetic diseases such as any set forth in Table 5.

TABLE 5
Exemplary Genes controlling Cellular Functions

| CELLULAR FUNCTION | GENES |
|---|---|
| PI3K/AKT Signaling | PRKCE; ITGAM; ITGA5; IRAK1; PRKAA2; EIF2AK2; PTEN; EIF4E; |
| | PRKCZ; GRK6; MAPK1; TSC1; PLK1; AKT2; IKBKB; PIK3CA; CDK8; |
| | CDKN1B; NFKB2; BCL2; PIK3CB; PPP2R1A; MAPK8; BCL2L1; MAPK3; |
| | TSC2; |
| | ITGA1; KRAS; EIF4EBP1; RELA; PRKCD; NOS3; |
| | PRKAA1; MAPK9; CDK2; PPP2CA; PIM1; ITGB7; |
| | YWHAZ; ILK; TP53; RAF1; IKBKG; RELB; DYRK1A; |
| | CDKN1A; ITGB1; MAP2K2; JAK1; AKT1; JAK2; PIK3R1; |
| | CHUK; PDPK1; PPP2R5C; CTNNB1; MAP2K1; NFKB1; |
| | PAK3; ITGB3; CCND1; GSK3A; FRAP1; SFN; ITGA2; |
| | TTK; CSNK1A1; BRAF; GSK3B; AKT3; FOXO1; SGK; |
| | HSP90AA1; RPS6KB1 |
| ERK/MAPK Signaling | PRKCE; ITGAM; ITGA5; HSPB1; IRAK1; PRKAA2; |
| | EIF2AK2; RAC1; RAP1A; TLN1; EIF4E; ELK1; GRK6; |
| | MAPK1; RAC2; PLK1; AKT2; PIK3CA; CDK8; CREB1; |
| | PRKC1; PTK2; FOS; RPS6KA4; PIK3CB; PPP2R1A; |
| | PIK3C3; MAPK8; MAPK3; ITGA1; ETS1; KRAS; MYCN; |
| | EIF4EBP1; PPARG; PRKCD; PRKAA1; MAPK9; SRC; |
| | CDK2; PPP2CA; PIM1; PIK3C2A; ITGB7; YWHAZ; |
| | PPP1CC; KSR1; PXN; RAF1; FYN; DYRK1A; ITGB1; |
| | MAP2K2; PAK4; PIK3R1; STAT3; PPP2R5C; MAP2K1; |
| | PAK3; ITGB3; ESR1; ITGA2; MYC; TTK; CSNK1A1; |
| | CRKL; BRAF; ATF4; PRKCA; SRF; STAT1; SGK |
| Glucocorticoid Receptor | RAC1; TAF4B; EP300; SMAD2; TRAF6; PCAF; ELK1; |
| Signaling | MAPK1; SMAD3; AKT2; IKBKB; NCOR2; UBE21; |
| | PIK3CA; CREB1; FOS; HSPA5; NFKB2; BCL2; |
| | MAP3K14; STAT5B; PIK3CB; PIK3C3; MAPK8; BCL2L1; |
| | MAPK3; TSC22D3; MAPK10; NRIP1; KRAS; MAPK13; |
| | RELA; STAT5A; MAPK9; NOS2A; PBX1; NR3C1; |
| | PIK3C2A; CDKN1C; TRAF2; SERPINE1; NCOA3; |
| | MAPK14; TNF; RAF1; IKBKG; MAP3K7; CREBBP; |
| | CDKN1A; MAP2K2; JAK1; IL8; NCOA2; AKT1; JAK2; |
| | PIK3R1; CHUK; STAT3; MAP2K1; NFKB1; TGFBR1; |
| | ESR1; SMAD4; CEBPB; JUN; AR; AKT3; CCL2; MMP1; |
| | STAT1; IL6; HSP90AA1 |
| Axonal Guidance Signaling | PRKCE; ITGAM; ROCK1; ITGA5; CXCR4; ADAM12; |
| | IGF1; RAC1; RAP1A; EIF4E; PRKCZ; NRP1; NTRK2; |
| | ARHGEF7; SMO; ROCK2; MAPK1; PGF; RAC2; |
| | PTPN11; GNAS; AKT2; PIK3CA; ERBB2; PRKCI; PTK2; |
| | CFL1; GNAQ; PIK3CB; CXCL12; PIK3C3; WNT11; |
| | PRKD1; GNB2L1; ABL1; MAPK3; ITGA1; KRAS; RHOA; |
| | PRKCD; PIK3C2A; ITGB7; GLI2; PXN; VASP; RAF1; |
| | FYN; ITGB1; MAP2K2; PAK4; ADAM17; AKT1; PIK3R1; |
| | GLI1; WNT5A; ADAM10; MAP2K1; PAK3; ITGB3; |
| | CDC42; VEGFA; ITGA2; EPHA8; CRKL; RND1; GSK3B; |
| | AKT3; PRKCA |
| Ephrin Receptor Signaling | PRKCE; ITGAM; ROCK1; ITGA5; CXCR4; IRAK1; |
| Actin Cytoskeleton | PRKAA2; EIF2AK2; RAC1; RAP1A; GRK6; ROCK2; |
| Signaling | MAPK1; PGF; RAC2; PTPN11; GNAS; PLK1; AKT2; |
| | DOK1; CDK8; CREB1; PTK2; CFL1; GNAQ; MAP3K14; |
| | CXCL12; MAPK8; GNB2L1; ABL1; MAPK3; ITGA1; |
| | KRAS; RHOA; PRKCD; PRKAA1; MAPK9; SRC; CDK2; |
| | PIM1; ITGB7; PXN; RAF1; FYN; DYRK1A; ITGB1; |
| | MAP2K2; PAK4; AKT1; JAK2; STAT3; ADAM10; |
| | MAP2K1; PAK3; ITGB3; CDC42; VEGFA; ITGA2; |
| | EPHA8; TTK; CSNK1A1; CRKL; BRAF; PTPN13; ATF4; |
| | AKT3; SGK |
| | ACTN4; PRKCE; ITGAM; ROCK1; ITGA5; IRAK1; |
| | PRKAA2; EIF2AK2; RAC1; INS; ARHGEF7; GRK6; |
| | ROCK2; MAPK1; RAC2; PLK1; AKT2; PIK3CA; CDK8; |
| | PTK2; CFL1; PIK3CB; MYH9; DIAPH1; PIK3C3; MAPK8; |
| | F2R; MAPK3; SLC9A1; ITGA1; KRAS; RHOA; PRKCD; |
| | PRKAA1; MAPK9; CDK2; PIM1; PIK3C2A; ITGB7; |
| | PPP1CC; PXN; VIL2; RAF1; GSN; DYRK1A; ITGB1; |
| | MAP2K2; PAK4; PIP5K1A; PIK3R1; MAP2K1; PAK3; |
| | ITGB3; CDC42; APC; ITGA2; TTK; CSNKIA1; CRKL; |
| | BRAF; VAV3; SGK |
| Huntington's Disease | PRKCE; IGF1; EP300; RCOR1; PRKCZ; HDAC4; TGM2; |
| Signaling | MAPK1; CAPNS1; AKT2; EGFR; NCOR2; SP1; CAPN2; |
| | PIK3CA; HDAC5; CREB1; PRKCI; HSPA5; REST; |
| | GNAQ; PIK3CB; PIK3C3; MAPK8; IGF1R; PRKD1; |
| | GNB2L1; BCL2L1; CAPN1; MAPK3; CASP8; HDAC2; |
| | HDAC7A; PRKCD; HDAC11; MAPK9; HDAC9; PIK3C2A; |
| | HDAC3; TP53; CASP9; CREBBP; AKT1; PIK3R1; |
| | PDPK1; CASP1; APAF1; FRAP1; CASP2; JUN; BAX; |
| | ATF4; AKT3; PRKCA; CLTC; SGK; HDAC6; CASP3 |
| Apoptosis Signaling | PRKCE; ROCK1; BID; IRAK1; PRKAA2; EIF2AK2; BAK1; |
| | BIRC4; GRK6; MAPK1; CAPNS1; PLK1; AKT2; IKBKB; |
| | CAPN2; CDK8; FAS; NFKB2; BCL2; MAP3K14; MAPK8; |
| | BCL2L1; CAPN1; MAPK3; CASP8; KRAS; RELA; |
| | PRKCD; PRKAA1; MAPK9; CDK2; PIM1; TP53; TNF; |
| | RAF1; IKBKG; RELB; CASP9; DYRK1A; MAP2K2; |
| | CHUK; APAF1; MAP2K1; NFKB1; PAK3; LMNA; CASP2; |
| | BIRC2; TTK; CSNK1A1; BRAF; BAX; PRKCA; SGK; |
| | CASP3; BIRC3; PARP1 |
| B Cell Receptor Signaling | RAC1; PTEN; LYN; ELK1; MAPK1; RAC2; PTPN11; |
| | AKT2; IKBKB; PIK3CA; CREB1; SYK; NFKB2; CAMK2A; |
| | MAP3K14; PIK3CB; PIK3C3; MAPK8; BCL2L1; ABL1; |
| | MAPK3; ETS1; KRAS; MAPK13; RELA; PTPN6; MAPK9; |
| | EGR1; PIK3C2A; BTK; MAPK14; RAF1; IKBKG; RELB; |
| | MAP3K7; MAP2K2; AKT1; PIK3R1; CHUK; MAP2K1; |
| | NFKB1; CDC42; GSK3A; FRAP1; BCL6; BCL10; JUN; |
| | GSK3B; ATF4; AKT3; VAV3; RPS6KB1 |
| Leukocyte Extravasation | ACTN4; CD44; PRKCE; ITGAM; ROCK1; CXCR4; CYBA; |
| Signaling | RAC1; RAP1A; PRKCZ; ROCK2; RAC2; PTPN11; |
| | MMP14; PIK3CA; PRKCI; PTK2; PIK3CB; CXCL12; |
| | PIK3C3; MAPK8; PRKD1; ABL1; MAPK10; CYBB; |
| | MAPK13; RHOA; PRKCD; MAPK9; SRC; PIK3C2A; BTK; |
| | MAPK14; NOX1; PXN; VIL2; VASP; ITGB1; MAP2K2; |
| | CTNND1; PIK3R1; CTNNB1; CLDN1; CDC42; F11R; ITK; |
| | CRKL; VAV3; CTTN; PRKCA; MMP1; MMP9 |
| Integrin Signaling | ACTN4; ITGAM; ROCK1; ITGA5; RAC1; PTEN; RAPIA; |
| | TLN1; ARHGEF7; MAPK1; RAC2; CAPNS1; AKT2; |
| | CAPN2; PIK3CA; PTK2; PIK3CB; PIK3C3; MAPK8; |
| | CAV1; CAPN1; ABL1; MAPK3; ITGA1; KRAS; RHOA; |
| | SRC; PIK3C2A; ITGB7; PPP1CC; ILK; PXN; VASP; |
| | RAF1; FYN; ITGB1; MAP2K2; PAK4; AKT1; PIK3R1; |
| | TNK2; MAP2K1; PAK3; ITGB3; CDC42; RND3; ITGA2; |
| | CRKL; BRAF; GSK3B; AKT3 |
| Acute Phase Response | IRAK1; SOD2; MYD88; TRAF6; ELK1; MAPK1; PTPN11; |
| Signaling | AKT2; IKBKB; PIK3CA; FOS; NFKB2; MAP3K14; |
| | PIK3CB; MAPK8; RIPK1; MAPK3; IL6ST; KRAS; |
| | MAPK13; IL6R; RELA; SOCS1; MAPK9; FTL; NR3C1; |
| | TRAF2; SERPINE1; MAPK14; TNF; RAF1; PDK1; |
| | IKBKG; RELB; MAP3K7; MAP2K2; AKT1; JAK2; PIK3R1; |
| | CHUK; STAT3; MAP2K1; NFKB1; FRAP1; CEBPB; JUN; |
| | AKT3; ILIR1; IL6 |
| PTEN Signaling | ITGAM; ITGA5; RAC1; PTEN; PRKCZ; BCL2L11; |
| | MAPK1; RAC2; AKT2; EGFR; IKBKB; CBL; PIK3CA; |
| | CDKNIB; PTK2; NFKB2; BCL2; PIK3CB; BCL2L1; |
| | MAPK3; ITGA1; KRAS; ITGB7; ILK; PDGFRB; INSR; |
| | RAF1; IKBKG; CASP9; CDKN1A; ITGB1; MAP2K2; |
| | AKT1; PIK3R1; CHUK; PDGFRA; PDPK1; MAP2K1; |
| | NFKB1; ITGB3; CDC42; CCND1; GSK3A; ITGA2; |
| | GSK3B; AKT3; FOXO1; CASP3; RPS6KB1 |
| p53 Signaling | PTEN; EP300; BBC3; PCAF; FASN; BRCA1; GADD45A; |
| Aryl Hydrocarbon Receptor | BIRC5; AKT2; PIK3CA; CHEK1; TP53INP1; BCL2; |
| Signaling | PIK3CB; PIK3C3; MAPK8; THBS1; ATR; BCL2L1; E2F1; |
| | PMAIP1; CHEK2; TNFRSF10B; TP73; RB1; HDAC9; |
| | CDK2; PIK3C2A; MAPK14; TP53; LRDD; CDKN1A; |
| | HIPK2; AKT1; PIK3R1; RRM2B; APAF1; CTNNB1; |
| | SIRT1; CCND1; PRKDC; ATM; SFN; CDKN2A; JUN; |
| | SNAI2; GSK3B; BAX; AKT3 |
| | HSPB1; EP300; FASN; TGM2; RXRA; MAPK1; NQO1; |
| | NCOR2; SP1; ARNT; CDKN1B; FOS; CHEK1; |
| | SMARCA4; NFKB2; MAPK8; ALDH1A1; ATR; E2F1; |
| | MAPK3; NRIP1; CHEK2; RELA; TP73; GSTP1; RB1; |
| | SRC; CDK2; AHR; NFE2L2; NCOA3; TP53; TNF; |
| | CDKN1A; NCOA2; APAF1; NFKB1; CCND1; ATM; ESR1; |
| | CDKN2A; MYC; JUN; ESR2; BAX; IL6; CYP1B1; |
| | HSP90AA1 |
| Xenobiotic Metabolism | PRKCE; EP300; PRKCZ; RXRA; MAPK1; NQO1; |
| Signaling | NCOR2; PIK3CA; ARNT; PRKC1; NFKB2; CAMK2A; |
| | PIK3CB; PPP2R1A; PIK3C3; MAPK8; PRKD1; |
| | ALDHIA1; MAPK3; NRIP1; KRAS; MAPK13; PRKCD; |
| | GSTP1; MAPK9; NOS2A; ABCB1; AHR; PPP2CA; FTL; |
| | NFE2L2; PIK3C2A; PPARGCIA; MAPK14; TNF; RAF1; |
| | CREBBP; MAP2K2; PIK3R1; PPP2R5C; MAP2K1; |
| | NFKB1; KEAP1; PRKCA; EIF2AK3; IL6; CYP1B1; |
| | HSP90AA1 |
| SAPK/JNK Signaling | PRKCE; IRAK1; PRKAA2; EIF2AK2; RAC1; ELK1; |
| | GRK6; MAPK1; GADD45A; RAC2; PLK1; AKT2; PIK3CA; |
| | FADD; CDK8; PIK3CB; PIK3C3; MAPK8; RIPK1; |
| | GNB2L1; IRS1; MAPK3; MAPK10; DAXX; KRAS; |
| | PRKCD; PRKAA1; MAPK9; CDK2; PIM1; PIK3C2A; |
| | TRAF2; TP53; LCK; MAP3K7; DYRK1A; MAP2K2; |
| | PIK3R1; MAP2K1; PAK3; CDC42; JUN; TTK; CSNK1A1; |
| | CRKL; BRAF; SGK |
| PPAr/RXR Signaling | PRKAA2; EP300; INS; SMAD2; TRAF6; PPARA; FASN; |
| | RXRA; MAPK1; SMAD3; GNAS; IKBKB; NCOR2; |
| | ABCA1; GNAQ; NFKB2; MAP3K14; STAT5B; MAPK8; |
| | IRS1; MAPK3; KRAS; RELA; PRKAA1; PPARGC1A; |
| | NCOA3; MAPK14; INSR; RAF1; IKBKG; RELB; MAP3K7; |
| | CREBBP; MAP2K2; JAK2; CHUK; MAP2K1; NFKB1; |
| | TGFBR1; SMAD4; JUN; IL1R1; PRKCA; IL6; HSP90AA1; |
| | ADIPOQ |
| NF-KB Signaling | IRAK1; EIF2AK2; EP300; INS; MYD88; PRKCZ; TRAF6; |
| | TBK1; AKT2; EGFR; IKBKB; PIK3CA; BTRC; NFKB2; |
| | MAP3K14; PIK3CB; PIK3C3; MAPK8; RIPK1; HDAC2; |
| | KRAS; RELA; PIK3C2A; TRAF2; TLR4; PDGFRB; TNF; |
| | INSR; LCK; IKBKG; RELB; MAP3K7; CREBBP; AKT1; |
| | PIK3R1; CHUK; PDGFRA; NFKB1; TLR2; BCL10; |
| | GSK3B; AKT3; TNFAIP3; ILIR1 |
| Neuregulin Signaling | ERBB4; PRKCE; ITGAM; ITGA5; PTEN; PRKCZ; ELK1; |
| Wnt & Beta catenin | MAPK1; PTPN11; AKT2; EGFR; ERBB2; PRKCI; |
| Signaling | CDKN1B; STAT5B; PRKD1; MAPK3; ITGA1; KRAS; |
| | PRKCD; STAT5A; SRC; ITGB7; RAF1; ITGB1; MAP2K2; |
| | ADAM17; AKT1; PIK3R1; PDPK1; MAP2K1; ITGB3; |
| | EREG; FRAP1; PSEN1; ITGA2; MYC; NRG1; CRKL; |
| | AKT3; PRKCA; HSP90AA1; RPS6KB1 |
| | CD44; EP300; LRP6; DVL3; CSNK1E; GJA1; SMO; |
| | AKT2; PIN1; CDH1; BTRC; GNAQ; MARK2; PPP2R1A; |
| | WNT11; SRC; DKK1; PPP2CA; SOX6; SFRP2; ILK; |
| | LEF1; SOX9; TP53; MAP3K7; CREBBP; TCF7L2; AKT1; |
| | PPP2R5C; WNT5A; LRP5; CTNNB1; TGFBR1; CCND1; |
| | GSK3A; DVL1; APC; CDKN2A; MYC; CSNK1A1; GSK3B; |
| | AKT3; SOX2 |
| Insulin Receptor Signaling | PTEN; INS; EIF4E; PTPN1; PRKCZ; MAPK1; TSC1; |
| | PTPN11; AKT2; CBL; PIK3CA; PRKCI; PIK3CB; PIK3C3; |
| | MAPK8; IRS1; MAPK3; TSC2; KRAS; EIF4EBP1; |
| | SLC2A4; PIK3C2A; PPP1CC; INSR; RAF1; FYN; |
| | MAP2K2; JAK1; AKT1; JAK2; PIK3R1; PDPK1; MAP2K1; |
| | GSK3A; FRAP1; CRKL; GSK3B; AKT3; FOXO1; SGK; |
| | RPS6KB1 |
| IL-6 Signaling | HSPB1; TRAF6; MAPKAPK2; ELK1; MAPK1; PTPN11; |
| | IKBKB; FOS; NFKB2; MAP3K14; MAPK8; MAPK3; |
| | MAPK10; IL6ST; KRAS; MAPK13; IL6R; RELA; SOCS1; |
| | MAPK9; ABCB1; TRAF2; MAPK14; TNF; RAF1; IKBKG; |
| | RELB; MAP3K7; MAP2K2; IL8; JAK2; CHUK; STAT3; |
| | MAP2K1; NFKB1; CEBPB; JUN; IL1R1; SRF; IL6 |
| Hepatic Cholestasis | PRKCE; IRAK1; INS; MYD88; PRKCZ; TRAF6; PPARA; |
| | RXRA; IKBKB; PRKC1; NFKB2; MAP3K14; MAPK8; |
| | PRKD1; MAPK10; RELA; PRKCD; MAPK9; ABCB1; |
| | TRAF2; TLR4; TNF; INSR; IKBKG; RELB; MAP3K7; IL8; |
| | CHUK; NR1H2; TJP2; NFKB1; ESR1; SREBF1; FGFR4; |
| | JUN; IL1R1; PRKCA; IL6 |
| IGF-1 Signaling | IGF1; PRKCZ; ELK1; MAPK1; PTPN11; NEDD4; AKT2; |
| | PIK3CA; PRKCI; PTK2; FOS; PIK3CB; PIK3C3; MAPK8; |
| | IGF1R; IRS1; MAPK3; IGFBP7; KRAS; PIK3C2A; |
| | YWHAZ; PXN; RAF1; CASP9; MAP2K2; AKT1; PIK3R1; |
| | PDPK1; MAP2K1; IGFBP2; SFN; JUN; CYR61; AKT3; |
| | FOXO1; SRF; CTGF; RPS6KB1 |
| NRF2-mediated Oxidative | PRKCE; EP300; SOD2; PRKCZ; MAPK1; SQSTM1; |
| Stress Response | NQO1; PIK3CA; PRKC1; FOS; PIK3CB; PIK3C3; MAPK8; |
| | PRKD1; MAPK3; KRAS; PRKCD; GSTP1; MAPK9; FTL; |
| | NFE2L2; PIK3C2A; MAPK14; RAF1; MAP3K7; CREBBP; |
| | MAP2K2; AKT1; PIK3R1; MAP2K1; PPIB; JUN; KEAP1; |
| | GSK3B; ATF4; PRKCA; EIF2AK3; HSP90AA1 |
| Hepatic Fibrosis/Hepatic | EDN1; IGF1; KDR; FLT1; SMAD2; FGFR1; MET; PGF; |
| Stellate Cell Activation | SMAD3; EGFR; FAS; CSF1; NFKB2; BCL2; MYH9; |
| | IGF1R; IL6R; RELA; TLR4; PDGFRB; TNF; RELB; IL8; |
| | PDGFRA; NFKB1; TGFBR1; SMAD4; VEGFA; BAX; |
| | IL1R1; CCL2; HGF; MMP1; STAT1; IL6; CTGF; MMP9 |
| PPAR Signaling | EP300; INS; TRAF6; PPARA; RXRA; MAPK1; IKBKB; |
| | NCOR2; FOS; NFKB2; MAP3K14; STAT5B; MAPK3; |
| | NRIP1; KRAS; PPARG; RELA; STAT5A; TRAF2; |
| | PPARGC1A; PDGFRB; TNF; INSR; RAF1; IKBKG; |
| | RELB; MAP3K7; CREBBP; MAP2K2; CHUK; PDGFRA; |
| | MAP2K1; NFKB1; JUN; IL1R1; HSP90AA1 |
| Fc Epsilon RI Signaling | PRKCE; RAC1; PRKCZ; LYN; MAPK1; RAC2; PTPN11; |
| | AKT2; PIK3CA; SYK; PRKC1; PIK3CB; PIK3C3; MAPK8; |
| | PRKD1; MAPK3; MAPK10; KRAS; MAPK13; PRKCD; |
| | MAPK9; PIK3C2A; BTK; MAPK14; TNF; RAF1; FYN; |
| | MAP2K2; AKT1; PIK3R1; PDPK1; MAP2K1; AKT3; |
| | VAV3; PRKCA |
| G-Protein Coupled | PRKCE; RAP1A; RGS16; MAPK1; GNAS; AKT2; IKBKB; |
| Receptor Signaling | PIK3CA; CREB1; GNAQ; NFKB2; CAMK2A; PIK3CB; |
| | PIK3C3; MAPK3; KRAS; RELA; SRC; PIK3C2A; RAF1; |
| | IKBKG; RELB; FYN; MAP2K2; AKT1; PIK3R1; CHUK; |
| | PDPK1; STAT3; MAP2K1; NFKB1; BRAF; ATF4; AKT3; |
| | PRKCA |
| Inositol Phosphate | PRKCE; IRAK1; PRKAA2; EIF2AK2; PTEN; GRK6; |
| Metabolism | MAPK1; PLK1; AKT2; PIK3CA; CDK8; PIK3CB; PIK3C3; |
| | MAPK8; MAPK3; PRKCD; PRKAA1; MAPK9; CDK2; |
| | PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; |
| | PIK3C3; MAPK8; CAV1; ABL1; MAPK3; KRAS; SRC; |
| | PIK3C2A; PDGFRB; RAF1; MAP2K2; JAK1; JAK2; |
| | PIK3R1; PDGFRA; STAT3; SPHK1; MAP2K1; MYC; |
| | JUN; CRKL; PRKCA; SRF; STAT1; SPHK2 |
| VEGF Signaling | ACTN4; ROCK1; KDR; FLT1; ROCK2; MAPK1; PGF; |
| | AKT2; PIK3CA; ARNT; PTK2; BCL2; PIK3CB; PIK3C3; |
| | BCL2L1; MAPK3; KRAS; HIF1A; NOS3; PIK3C2A; PXN; |
| | RAF1; MAP2K2; ELAVL1; AKT1; PIK3R1; MAP2K1; SFN; |
| | VEGFA; AKT3; FOXO1; PRKCA |
| Natural Killer Cell Signaling | PRKCE; RAC1; PRKCZ; MAPK1; RAC2; PTPN11; |
| | KIR2DL3; AKT2; PIK3CA; SYK; PRKCI; PIK3CB; |
| | PIK3C3; PRKD1; MAPK3; KRAS; PRKCD; PTPN6; |
| | PIK3C2A; LCK; RAF1; FYN; MAP2K2; PAK4; AKT1; |
| | PIK3R1; MAP2K1; PAK3; AKT3; VAV3; PRKCA |
| Cell Cycle: G1/S | HDAC4; SMAD3; SUV39H1; HDAC5; CDKN1B; BTRC; |
| Checkpoint Regulation | ATR; ABL1; E2F1; HDAC2; HDAC7A; RB1; HDAC11; |
| | HDAC9; CDK2; E2F2; HDAC3; TP53; CDKN1A; CCND1; |
| | E2F4; ATM; RBL2; SMAD4; CDKN2A; MYC; NRG1; |
| | GSK3B; RBL1; HDAC6 |
| T Cell Receptor Signaling | RAC1; ELK1; MAPK1; IKBKB; CBL; PIK3CA; FOS; |
| | NFKB2; PIK3CB; PIK3C3; MAPK8; MAPK3; KRAS; |
| | RELA; PIK3C2A; BTK; LCK; RAF1; IKBKG; RELB; FYN; |
| | MAP2K2; PIK3R1; CHUK; MAP2K1; NFKB1; ITK; BCL10; |
| | JUN; VAV3 |
| Death Receptor Signaling | CRADD; HSPB1; BID; BIRC4; TBK1; IKBKB; FADD; |
| | FAS; NFKB2; BCL2; MAP3K14; MAPK8; RIPK1; CASP8; |
| | DAXX; TNFRSF10B; RELA; TRAF2; TNF; IKBKG; RELB; |
| | CASP9; CHUK; APAF1; NFKB1; CASP2; BIRC2; CASP3; |
| | BIRC3 |
| FGF Signaling | RAC1; FGFR1; MET; MAPKAPK2; MAPK1; PTPN11; |
| | AKT2; PIK3CA; CREB1; PIK3CB; PIK3C3; MAPK8; |
| | MAPK3; MAPK13; PTPN6; PIK3C2A; MAPK14; RAF1; |
| | AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; |
| | STAT5B; PIK3CB; PIK3C3; GNB2L1; BCL2L1; MAPK3; |
| | ETS1; KRAS; RUNX1; PIM1; PIK3C2A; RAF1; MAP2K2; |
| | AKT1; JAK2; PIK3R1; STAT3; MAP2K1; CCND1; AKT3; |
| | STAT1 |
| Amyotrophic Lateral | BID; IGF1; RAC1; BIRC4; PGF; CAPNS1; CAPN2; |
| Sclerosis Signaling | PIK3CA; BCL2; PIK3CB; PIK3C3; BCL2L1; CAPN1; |
| | PIK3C2A; TP53; CASP9; PIK3R1; RAB5A; CASP1; |
| | APAF1; VEGFA; BIRC2; BAX; AKT3; CASP3; BIRC3 |
| JAK/Stat Signaling | PTPN1; MAPK1; PTPN11; AKT2; PIK3CA; STAT5B; |
| | PIK3CB; PIK3C3; MAPK3; KRAS; SOCS1; STAT5A; |
| | PTPN6; PIK3C2A; RAF1; CDKN1A; MAP2K2; JAK1; |
| | AKT1; JAK2; PIK3R1; STAT3; MAP2K1; FRAP1; AKT3; |
| | STAT1 |
| Nicotinate and Nicotinamide | PRKCE; IRAK1; PRKAA2; EIF2AK2; GRK6; MAPK1; |
| Metabolism | PLK1; AKT2; CDK8; MAPK8; MAPK3; PRKCD; PRKAA1; |
| | PBEF1; MAPK9; CDK2; PIM1; DYRK1A; MAP2K2; |
| | MAP2K1; PAK3; NT5E; TTK; CSNK1A1; BRAF; SGK |
| Chemokine Signaling | CXCR4; ROCK2; MAPK1; PTK2; FOS; CFL1; GNAQ; |
| | CAMK2A; CXCL12; MAPK8; MAPK3; KRAS; MAPK13; |
| | RHOA; CCR3; SRC; PPP1CC; MAPK14; NOX1; RAF1; |
| | MAP2K2; MAP2K1; JUN; CCL2; PRKCA |
| IL-2 Signaling | ELK1; MAPK1; PTPN11; AKT2; PIK3CA; SYK; FOS; |
| | STAT5B; PIK3CB; PIK3C3; MAPK8; MAPK3; KRAS; |
| | SOCS1; STAT5A; PIK3C2A; LCK; RAF1; MAP2K2; |
| | JAK1; AKT1; PIK3R1; MAP2K1; JUN; AKT3 |
| Synaptic Long Term | PRKCE; IGF1; PRKCZ; PRDX6; LYN; MAPK1; GNAS; |
| Depression | PRKCI; GNAQ; PPP2R1A; IGF1R; PRKD1; MAPK3; |
| | KRAS; GRN; PRKCD; NOS3; NOS2A; PPP2CA; |
| | YWHAZ; RAF1; MAP2K2; PPP2R5C; MAP2K1; PRKCA |
| Estrogen Receptor | TAF4B; EP300; CARM1; PCAF; MAPK1; NCOR2; |
| Signaling | SMARCA4; MAPK3; NRIP1; KRAS; SRC; NR3C1; |
| | HDAC3; PPARGC1A; RBM9; NCOA3; RAF1; CREBBP; |
| | MAP2K2; NCOA2; MAP2K1; PRKDC; ESR1; ESR2 |
| Protein Ubiquitination | TRAF6; SMURF1; BIRC4; BRCA1; UCHL1; NEDD4; |
| Pathway | CBL; UBE21; BTRC; HSPA5; USP7; USP10; FBXW7; |
| | USP9X; STUB1; USP22; B2M; BIRC2; PARK2; USP8; |
| | USP1; VHL; HSP90AA1; BIRC3 |
| IL-10 Signaling | TRAF6; CCR1; ELK1; IKBKB; SP1; FOS; NFKB2; |
| | MAP3K14; MAPK8; MAPK13; RELA; MAPK14; TNF; |
| | IKBKG; RELB; MAP3K7; JAK1; CHUK; STAT3; NFKB1; |
| | JUN; ILIR1; IL6 |
| VDR/RXR Activation | PRKCE; EP300; PRKCZ; RXRA; GADD45A; HES1; |
| | NCOR2; SP1; PRKC1; CDKN1B; PRKD1; PRKCD; |
| | RUNX2; KLF4; YY1; NCOA3; CDKN1A; NCOA2; SPP1; |
| | LRP5; CEBPB; FOXO1; PRKCA |
| TGF-beta Signaling | EP300; SMAD2; SMURF1; MAPK1; SMAD3; SMAD1; |
| | FOS; MAPK8; MAPK3; KRAS; MAPK9; RUNX2; |
| | SERPINE1; RAF1; MAP3K7; CREBBP; MAP2K2; |
| | MAP2K1; TGFBR1; SMAD4; JUN; SMAD5 |
| Toll-like Receptor Signaling | IRAK1; EIF2AK2; MYD88; TRAF6; PPARA; ELK1; |
| | IKBKB; FOS; NFKB2; MAP3K14; MAPK8; MAPK13; |
| | RELA; TLR4; MAPK14; IKBKG; RELB; MAP3K7; CHUK; |
| | NFKB1; TLR2; JUN |
| p38 MAPK Signaling | HSPB1; IRAK1; TRAF6; MAPKAPK2; ELK1; FADD; FAS; |
| | CREB1; DDIT3; RPS6KA4; DAXX; MAPK13; TRAF2; |
| | MAPK14; TNF; MAP3K7; TGFBR1; MYC; ATF4; IL1R1; |
| | SRF; STAT1 |
| Neurotrophin/TRK Signaling | NTRK2; MAPK1; PTPN11; PIK3CA; CREB1; FOS; |
| | PIK3CB; PIK3C3; MAPK8; MAPK3; KRAS; PIK3C2A; |
| | RAF1; MAP2K2; AKT1; PIK3R1; PDPK1; MAP2K1; |
| | CDC42; JUN; ATF4 |
| FXR/RXR Activation | INS; PPARA; FASN; RXRA; AKT2; SDC1; MAPK8; |
| | APOB; MAPK10; PPARG; MTTP; MAPK9; PPARGC1A; |
| | TNF; CREBBP; AKT1; SREBF1; FGFR4; AKT3; FOXO1 |
| Synaptic Long Term | PRKCE; RAP1A; EP300; PRKCZ; MAPK1; CREB1; |
| Potentiation | PRKCI; GNAQ; CAMK2A; PRKD1; MAPK3; KRAS; |
| | PRKCD; PPP1CC; RAF1; CREBBP; MAP2K2; MAP2K1; |
| | ATF4; PRKCA |
| Calcium Signaling | RAP1A; EP300; HDAC4; MAPK1; HDAC5; CREB1; |
| | CAMK2A; MYH9; MAPK3; HDAC2; HDAC7A; HDAC11; |
| | HDAC9; HDAC3; CREBBP; CALR; CAMKK2; ATF4; |
| | HDAC6 |
| EGF Signaling | ELK1; MAPK1; EGFR; PIK3CA; FOS; PIK3CB; PIK3C3; |
| | MAPK8; MAPK3; PIK3C2A; RAF1; JAK1; PIK3R1; |
| | STAT3; MAP2K1; JUN; PRKCA; SRF; STAT1 |
| Hypoxia Signaling in the | EDN1; PTEN; EP300; NQO1; UBE2I; CREB1; ARNT; |
| Cardiovascular System | HIF1A; SLC2A4; NOS3; TP53; LDHA; AKT1; ATM; |
| | VEGFA; JUN; ATF4; VHL; HSP90AA1 |
| LPS/IL-1 Mediated Inhibition | IRAK1; MYD88; TRAF6; PPARA; RXRA; ABCA1; |
| of RXR Function | MAPK8; ALDH1A1; GSTP1; MAPK9; ABCB1; TRAF2; |
| | TLR4; TNF; MAP3K7; NR1H2; SREBF1; JUN; IL1R1 |
| LXR/RXR Activation | FASN; RXRA; NCOR2; ABCA1; NFKB2; IRF3; RELA; |
| | NOS2A; TLR4; TNF; RELB; LDLR; NR1H2; NFKB1; |
| | SREBF1; IL1R1; CCL2; IL6; MMP9 |
| Amyloid Processing | PRKCE; CSNKIE; MAPK1; CAPNS1; AKT2; CAPN2; |
| | CAPN1; MAPK3; MAPK13; MAPT; MAPK14; AKT1; |
| | PSEN1; CSNK1A1; GSK3B; AKT3; APP |
| IL-4 Signaling | AKT2; PIK3CA; PIK3CB; PIK3C3; IRS1; KRAS; SOCS1; |
| | PTPN6; NR3C1; PIK3C2A; JAK1; AKT1; JAK2; PIK3R1; |
| | FRAP1; AKT3; RPS6KB1 |
| Cell Cycle: G2/M DNA | EP300; PCAF; BRCA1; GADD45A; PLK1; BTRC; |
| Damage Checkpoint | CHEK1; ATR; CHEK2; YWHAZ; TP53; CDKN1A; |
| Regulation | PRKDC; ATM; SFN; CDKN2A |
| Nitric Oxide Signaling in the | KDR; FLT1; PGF; AKT2; PIK3CA; PIK3CB; PIK3C3; |
| Cardiovascular System | CAV1; PRKCD; NOS3; PIK3C2A; AKT1; PIK3R1; |
| | VEGFA; AKT3; HSP90AA1 |
| Purine Metabolism | NME2; SMARCA4; MYH9; RRM2; ADAR; EIF2AK4; |
| | PKM2; ENTPD1; RAD51; RRM2B; TJP2; RAD51C; |
| | NT5E; POLD1; NME1 |
| cAMP-mediated Signaling | RAP1A; MAPK1; GNAS; CREB1; CAMK2A; MAPK3; |
| | SRC; RAF1; MAP2K2; STAT3; MAP2K1; BRAF; ATF4 |
| Mitochondrial Dysfunction | SOD2; MAPK8; CASP8; MAPK10; MAPK9; CASP9; |
| Notch Signaling | PARK7; PSEN1; PARK2; APP; CASP3 |
| | HES1; JAG1; NUMB; NOTCH4; ADAM17; NOTCH2; |
| | PSEN1; NOTCH3; NOTCH1; DLL4 |
| Endoplasmic Reticulum | HSPA5; MAPK8; XBP1; TRAF2; ATF6; CASP9; ATF4; |
| Stress Pathway | EIF2AK3; CASP3 |
| Pyrimidine Metabolism | NME2; AICDA; RRM2; EIF2AK4; ENTPD1; RRM2B; |
| | NT5E; POLD1; NME1 |
| Parkinson's Signaling | UCHL1; MAPK8; MAPK13; MAPK14; CASP9; PARK7; |
| | PARK2; CASP3 |
| Cardiac & Beta Adrenergic | GNAS; GNAQ; PPP2R1A; GNB2L1; PPP2CA; PPP1CC; |
| Signaling | PPP2R5C |
| Glycolysis/Gluconeogenesis | HK2; GCK; GP1; ALDH1A1; PKM2; LDHA; HK1 |
| Interferon Signaling | IRF1; SOCS1; JAK1; JAK2; IFITM1; STAT1; IFIT3 |
| Sonic Hedgehog Signaling | ARRB2; SMO; GLI2; DYRK1A; GLI1; GSK3B; DYRK1B |
| Glycerophospholipid | PLD1; GRN; GPAM; YWHAZ; SPHK1; SPHK2 |
| Metabolism | |
| Phospholipid Degradation | PRDX6; PLD1; GRN; YWHAZ; SPHK1; SPHK2 |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision Repair | ERCC5; ERCC4; XPA; XPC; ERCC1 |
| Pathway | |
| Starch and Sucrose | UCHL1; HK2; GCK; GP1; HK1 |
| Metabolism | |
| Aminosugars Metabolism | NQO1; HK2; GCK; HK1 |
| Arachidonic Acid | PRDX6; GRN; YWHAZ; CYP1B1 |
| Metabolism | |
| Circadian Rhythm Signaling | CSNK1E; CREB1; ATF4; NR1D1 |
| Coagulation System | BDKRB1; F2R; SERPINE1; F3 |
| Dopamine Receptor | PPP2R1A; PPP2CA; PPP1CC; PPP2R5C |
| Signaling | |
| Glutathione Metabolism | IDH2; GSTP1; ANPEP; IDH1 |
| Glycerolipid Metabolism | ALDH1A1; GPAM; SPHK1; SPHK2 |
| Linoleic Acid Metabolism | PRDX6; GRN; YWHAZ; CYP1B1 |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine and | PRDX6; PRDX1; TYR |
| Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of Xenobiotics | GSTP1; CYP1B1 |
| by Cytochrome p450 | |
| Methane Metabolism | PRDX6; PRDX1 |
| Phenylalanine Metabolism | PRDX6; PRDX1 |
| Propanoate Metabolism | ALDH1A1; LDHA |
| Selenoamino Acid | PRMT5; AHCY |
| Metabolism | |
| Sphingolipid Metabolism | SPHK1; SPHK2 |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated Oxidative | PRDX1 |
| Stress Response | |
| Pentose Phosphate | GPI |
| Pathway | |
| Pentose and Glucuronate | UCHL1 |
| Interconversions | |
| Retinol Metabolism | ALDH1A1 |
| Riboflavin Metabolism | TYR |
| Tyrosine Metabolism | PRMT5, TYR |
| Ubiquinone Biosynthesis | PRMT5 |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; cnr2; Grk2; |
| | Trpa1; Pomc; Cgrp; Crf; Pka; Era; Nr2b; TRPM5; Prkaca; |
| | Prkacb; Prkar1a; Prkar2a |
| Mitochondrial Function | AIF; CytC; SMAC (Diablo); Aifm-1; Aifm-2 |
| Developmental Neurology | BMP-4; Chordin (Chrd); Noggin (Nog); WNT (Wnt2; |
| | Wnt2b; Wnt3a; Wnt4; Wnt5a; Wnt6; Wnt7b; Wnt8b; |
| | Wnt9a; Wnt9b; Wnt10a; Wnt10b; Wnt16); beta-catenin; |
| | Dkk-1; Frizzled related proteins; Otx-2; Gbx2; FGF-8; |
| | Reelin; Dab1; unc-86 (Pou4f1 or Brn3a); Numb; Reln |

In an aspect, the invention provides a method of individualized or personalized treatment of a genetic disease in a subject in need of such treatment comprising: (a) introducing one or more mutations ex vivo in a tissue, organ or a cell line, or in vivo in a transgenic non-human mammal, comprising delivering to cell(s) of the tissue, organ, cell or mammal a composition comprising the particle delivery system or the delivery system or the virus particle of any one of the above embodiment or the cell of any one of the above embodiment, wherein the specific mutations or precise sequence substitutions are or have been correlated to the genetic disease; (b) testing treatment(s) for the genetic disease on the cells to which the vector has been delivered that have the specific mutations or precise sequence substitutions correlated to the genetic disease; and (c) treating the subject based on results from the testing of treatment(s) of step (b).

### Infectious Diseases

In one embodiment, the composition, system, (s) or component(s) thereof can be used to diagnose, prognose, treat, and/or prevent an infectious disease caused by a microorganism, such as bacteria, virus, fungi, parasites, or combinations thereof.

In one embodiment, the system(s) or component(s) thereof can be capable of targeting specific microorganism within a mixed population. Exemplary methods of such techniques are described in e.g. Gomaa A A, Klumpe H E, Luo M L, Selle K, Barrangou R, Beisel C L. 2014. Programmable removal of bacterial strains by use of genome-targeting composition, systems, mBio 5: e00928-13; Citorik R J, Mimee M, Lu T K. 2014. Sequence-specific antimicrobials using efficiently delivered RNA-guided nucleases. Nat Biotechnol 32:1141-1145, the teachings of which can be adapted for use with the compositions, systems, and components thereof described herein.

In one embodiment, the composition, system, (s) and/or components thereof can be capable of targeting pathogenic and/or drug-resistant microorganisms, such as bacteria, virus, parasites, and fungi. In one embodiment, the composition, system, (s) and/or components thereof can be capable of targeting and modifying one or more polynucleotides in a pathogenic microorganism such that the microorganism is less virulent, killed, inhibited, or is otherwise rendered incapable of causing disease and/or infecting and/or replicating in a host cell.

In one embodiment, the pathogenic bacteria that can be targeted and/or modified by the composition, system, (s) and/or component(s) thereof described herein include, but are not limited to, those of the genus Actinomyces (e.g. A. israelii), Bacillus (e.g. B. anthracis, B. cereus), Bactereoides (e.g. B. fragilis), Bartonella (B. henselae, B. quintana), Bordetella (B. pertussis), Borrelia (e.g. B. burgdorferi, B. garinii, B. afzelii, and B. recurreentis), Brucella (e.g. B. abortus, B. canis, B. melitensis, and B. suis), Campylobacter (e.g. C. jejuni), Chlamydia (e.g. C pneumoniae and C. trachomatis), Chlamydophila (e.g. C. psittaci), Clostridium (e.g. C. botulinum, C. difficile, C. perfringens. C. tetani), Corynebacterium (e.g. C. diptheriae), Enterococcus (e.g. E. Faecalis, E. faecium), Ehrlichia (E. canis and E. chaffensis) Escherichia (e.g. E. coli), Francisella (e.g. F. tularensis), Haemophilus (e.g. H. influenzae), Helicobacter (H. pylori), Klebsiella (E.g. K. pneumoniae), Legionella (e.g. L. pneumophila), Leptospira (e.g. L. interrogans, L. santarosai, L. weilii, L. noguchii), Listereia (e.g. L. monocytogeenes), Mycobacterium (e.g. M. leprae, M. tuberculosis, M. ulcerans), Mycoplasma (M. pneumoniae), Neisseria (N. gonorrhoeae and N. menigitidis), Nocardia (e.g. N. asteeroides), Pseudomonas (P. aeruginosa), Rickettsia (R. rickettsia), Salmonella (S. typhi and S. typhimurium), Shigella (S. sonnei and S. dysenteriae), Staphylococcus (S. aureus, S. epidermidis, and S. saprophyticus), Streptococcus (S. agalactiaee, S. pneumoniae, S. pyogenes), Treponema (T. pallidum), Ureeaplasma (e.g. (J. urealyticum), Vibrio (e.g. V. cholerae), Yersinia (e.g. Y. pestis, Y. enterocolitica, and Y. pseudotuberculosis).

In one embodiment, the pathogenic virus that can be targeted and/or modified by the composition, system(s), and/or component(s) thereof described herein include, but are not limited to, a double-stranded DNA virus, a partly double-stranded DNA virus, a single-stranded DNA virus, a positive single-stranded RNA virus, a negative single-stranded RNA virus, or a double stranded RNA virus. In one embodiment, the pathogenic virus can be from the family Adenoviridae (e.g. Adenovirus), Herpeesviridae (e.g. Herpes simplex, type 1, Herpes simplex, type 2, Varicella-zoster virus, Epstein-Barr virus, Human cytomegalovirus, Human herpesvirus, type 8), Papillomaviridae (e.g. Human papillomavirus), Polyomaviridae (e.g. BK virus, JC virus), Poxviridae (e.g. smallpox), Hepadnaviridae (e.g. Hepatitis B), Parvoviridae (e.g. Parvovirus B19), Astroviridae (e.g. Human astrovirus), Caliciviridae (e.g. Norwalk virus), Picornaviridae (e.g. coxsackievirus, hepatitis A virus, poliovirus, rhinovirus), Coronaviridae (e.g. Severe acute respiratory syndrome-related coronavirus, strains: Severe acute respiratory syndrome virus, Severe acute respiratory syndrome coronavirus 2 (COVID-19)), Flaviviridae (e.g. Hepatitis C virus, yellow fever virus, dengue virus, West Nile virus, TBE virus), Togaviridae (e.g. Rubella virus), Hepeviridae (e.g. Hepatitis E virus), Retroviridae (Human immunodeficiency virus (HIV)), Orthomyxoviridae (e.g. Influenza virus), Arenaviridae (e.g. Lassa virus), Bunyaviridae (e.g. Crimean-Congo hemorrhagic fever virus, Hantaan virus), Filoviridae (e.g. Ebola virus and Marburg virus), Paramyxoviridae (e.g. Measles virus, Mumps virus, Parainfluenza virus, Respiratory syncytial virus), Rhabdoviridae (Rabies virus), Hepatits D virus, Reoviridae (e.g. Rotavirus, Orbivirus, Coltivirus, Banna virus).

In one embodiment, the pathogenic fungi that can be targeted and/or modified by the composition, system, (s) and/or component(s) thereof described herein include, but are not limited to, those of the genus Candida (e.g. C. albicans), Aspergillus (e.g. A. fumigatus, A. flavus, A. clavatus), Cryptococcus (e.g. C. neoformans, C. gattii), Histoplasma (H. capsulatum), Pneumocystis (e.g. P. jiroveecii), Stachybotrys (e.g. S. chartarum).

In one embodiment, the pathogenic parasites that can be targeted and/or modified by the composition, system(s), and/or component(s) thereof described herein include, but are not limited to, protozoa, helminths, and ectoparasites. In one embodiment, the pathogenic protozoa that can be targeted and/or modified by the composition, system(s), and/or component(s) thereof described herein include, but are not limited to, those from the groups Sarcodina (e.g. ameba such as Entamoeba), Mastigophora (e.g. flagellates such as Giardia and Leishmania), Cilophora (e.g. ciliates such as Balantidum), and sporozoa (e.g. plasmodium and cryptosporidium). In one embodiment, the pathogenic helminths that can be targeted and/or modified by the composition, system(s), and/or component(s) thereof described herein include, but are not limited to, flatworms (platyhelminths), thorny-headed worms (acanthoceephalins), and roundworms (nematodes). In one embodiment, the pathogenic ectoparasites that can be targeted and/or modified by the composition, system(s), and/or component(s) thereof described herein include, but are not limited to, ticks, fleas, lice, and mites.

In one embodiment, the pathogenic parasite that can be targeted and/or modified by the composition, system, (s) and/or component(s) thereof described herein include, but are not limited to, Acanthamoeba spp., Balamuthia mandrillaris, Babesiosis spp. (e.g. Babesia B. divergens, B. bigemina, B. equi, B. microfti, B. duncani), Balantidiasis spp. (e.g. Balantidium coli), Blastocystis spp., Cryptosporidium spp., Cyclosporiasis spp. (e.g. Cyclospora cayetanensis), Dientamoebiasis spp. (e.g. Dientamoeba fragilis), Amoebiasis spp. (e.g. Entamoeba histolytica), Giardiasis spp. (e.g. Giardia lamblia), Isosporiasis spp. (e.g. Isospora belli), Leishmania spp., Naegleria spp. (e.g. Naegleria fowleri), Plasmodium spp. (e.g. Plasmodium falciparum, Plasmodium vivax, Plasmodium ovale curtisi, Plasmodium ovale wallikeri, Plasmodium malariae, Plasmodium knowlesi), Rhinosporidiosis spp. (e.g. Rhinosporidium seeberi), Sarcocystosis spp. (e.g. Sarcocystis bovihominis, Sarcocystis suihominis), Toxoplasma spp. (e.g. Toxoplasma gondii), Trichomonas spp. (e.g. Trichomonas vaginalis), Trypanosoma spp. (e.g. Trypanosoma brucei), Trypanosoma spp. (e.g. Trypanosoma cruzi), Tapeworm (e.g. Cestoda, Taenia multiceps, Taenia saginata, Taenia solium), Diphyllobothrium latum spp., Echinococcus spp. (e.g. Echinococcus granulosus, Echinococcus multilocularis, E. vogeli, E. oligarthrus), Hymenolepis spp. (e.g. Hymenolepis nana, Hymenolepis diminuta), Bertiella spp. (e.g. Bertiella mucronata, Bertiella studeri), Spirometra (e.g. Spirometra erinaceieuropaei), Clonorchis spp. (e.g. Clonorchis sinensis; Clonorchis viverrini), Dicrocoelium spp. (e.g. Dicrocoelium dendriticum), Fasciola spp. (e.g. Fasciola hepatica, Fasciola gigantica), Fasciolopsis spp. (e.g. Fasciolopsis buski), Metagonimus spp. (e.g. Metagonimus yokogawai), Metorchis spp. (e.g. Metorchis conjunctus), Opisthorchis spp. (e.g. Opisthorchis viverrini, Opisthorchis felineus), Clonorchis spp. (e.g. Clonorchis sinensis), Paragonimus spp. (e.g. Paragonimus westermani; Paragonimus africanus; Paragonimus caliensis; Paragonimus kellicotti; Paragonimus skrjabini; Paragonimus uterobilateralis), Schistosoma sp., Schistosoma spp. (e.g. Schistosoma mansoni, Schistosoma haematobium, Schistosoma japonicum, Schistosoma mekongi, and Schistosoma intercalatum), Echinostoma spp. (e.g. E. echinatum), Trichobilharzia spp. (e.g. Trichobilharzia regent), Ancylostoma spp. (e.g. Ancylostoma duodenale), Necator spp. (e.g. Necator americanus), Angiostrongylus spp., Anisakis spp., Ascaris spp. (e.g. Ascaris lumbricoides), Baylisascaris spp. (e.g. Baylisascaris procyonis), Brugia spp. (e.g. Brugia malayi, Brugia timori), Dioctophyme spp. (e.g. Dioctophyme renale), Dracunculus spp. (e.g. Dracunculus medinensis), Enterobius spp. (e.g. Enterobius vermicularis, Enterobius gregorii), Gnathostoma spp. (e.g. Gnathostoma spinigerum, Gnathostoma hispidum), Halicephalobus spp. (e.g. Halicephalobus gingivalis), Loa loa spp. (e.g. Loa loa filaria), Mansonella spp. (e.g. Mansonella streptocerca), Onchocerca spp. (e.g. Onchocerca volvulus), Strongyloides spp. (e.g. Strongyloides stercoralis), Thelazia spp. (e.g. Thelazia californiensis, Thelazia callipaeda), Toxocara spp. (e.g. Toxocara canis, Toxocara cati, Toxascaris leonine), Trichinella spp. (e.g. Trichinella spiralis, Trichinella britovi, Trichinella nelsoni, Trichinella nativa), Trichuris spp. (e.g. Trichuris trichiura, Trichuris vulpis), Wuchereria spp. (e.g. Wuchereria bancrofti), Dermatobia spp. (e.g. Dermatobia hominis), Tunga spp. (e.g. Tunga penetrans), Cochliomyia spp. (e.g. Cochliomyia hominivorax), Linguatula spp. (e.g. Linguatula serrata), Archiacanthocephala sp., Moniliformis sp. (e.g. Moniliformis moniliformis), Pediculus spp. (e.g. Pediculus humanus capitis, Pediculus humanus humanus), Pthirus spp. (e.g. Pthirus pubis), Arachnida spp. (e.g. Trombiculidae, Ixodidae, Argaside), Siphonaptera spp (e.g. Siphonaptera: Pulicinae), Cimicidae spp. (e.g. Cimex lectularius and Cimex hemipterus), Diptera spp., Demodex spp. (e.g. Demodex folliculorum brevis canis), Sarcoptes spp. (e.g. Sarcoptes scabiei), Dermanyssus spp. (e.g. Dermanyssus gallinae), Ornithonyssus spp. (e.g. Ornithonyssus sylviarum, Ornithonyssus bursa, Ornithonyssus bacoti), Laelaps spp. (e.g. Laelaps echidnina), Liponyssoides spp. (e.g. Liponyssoides sanguineus).

In one embodiment the gene targets can be any of those as set forth in Table 1 of Strich and Chertow. 2019. J. Clin. Microbio. 57:4 e01307-18, which is incorporated herein as if expressed in its entirety herein.

In one embodiment, the method can include delivering a composition, system, and/or component thereof to a pathogenic organism described herein, allowing the composition, system, and/or component thereof to specifically bind and modify one or more targets in the pathogenic organism, whereby the modification kills, inhibits, reduces the pathogenicity of the pathogenic organism, or otherwise renders the pathogenic organism non-pathogenic. In one embodiment, delivery of the composition, system, occurs in vivo (i.e. in the subject being treated). In one embodiment occurs by an intermediary, such as microorganism or phage that is non-pathogenic to the subject but is capable of transferring polynucleotides and/or infecting the pathogenic microorganism. In one embodiment, the intermediary microorganism can be an engineered bacteria, virus, or phage that contains the composition, system(s), and/or component(s) thereof and/or vectors and/or vector systems. The method can include administering an intermediary microorganism containing the composition, system(s), and/or component(s) thereof and/or vectors and/or vector systems to the subject to be treated. The intermediary microorganism can then produce the compositions and/or component thereof or transfer a composition, system, polynucleotide to the pathogenic organism. In embodiments, where the compositions and/or component thereof, vector, or vector system is transferred to the pathogenic microorganism, the composition, system, or component thereof is then produced in the pathogenic microorganism and modifies the pathogenic microorganism such that it is less virulent, killed, inhibited, or is otherwise rendered incapable of causing disease and/or infecting and/or replicating in a host or cell thereof.

In one embodiment, where the pathogenic microorganism inserts its genetic material into the host cell's genome (e.g. a virus), the composition, system, can be designed such that it modifies the host cell's genome such that the viral DNA or cDNA cannot be replicated by the host cell's machinery into a functional virus. In one embodiment, where the pathogenic microorganism inserts its genetic material into the host cell's genome (e.g. a virus), the composition, system, can be designed such that it modifies the host cell's genome such that the viral DNA or cDNA is deleted from the host cell's genome.

It will be appreciated that inhibiting or killing the pathogenic microorganism, the disease and/or condition that its infection causes in the subject can be treated or prevented. Thus, also provided herein are methods of treating and/or preventing one or more diseases or symptoms thereof caused by any one or more pathogenic microorganisms, such as any of those described herein.

### Mitochondrial Diseases

Some of the most challenging mitochondrial disorders arise from mutations in mitochondrial DNA (mtDNA), a high copy number genome that is maternally inherited. In one embodiment, mtDNA mutations can be modified using a composition, system, described herein. In one embodiment, the mitochondrial disease that can be diagnosed, prognosed, treated, and/or prevented can be MELAS (mitochondrial myopathy encephalopathy, and lactic acidosis and stroke-like episodes), CPEO/PEO (chronic progressive external ophthalmoplegia syndrome/progressive external ophthalmoplegia), KSS (Kearns-Sayre syndrome), MIDD (maternally inherited diabetes and deafness), MERRF (myoclonic epilepsy associated with ragged red fibers), NIDDM (noninsulin-dependent diabetes mellitus), LHON (Leber hereditary optic neuropathy), LS (Leigh Syndrome) an aminoglycoside induced hearing disorder, NARP (neuropathy, ataxia, and pigmentary retinopathy), Extrapyramidal disorder with akinesia-rigidity, psychosis and SNHL, Nonsyndromic hearing loss a cardiomyopathy, an encephalomyopathy, Pearson's syndrome, or a combination thereof.

In one embodiment, the mtDNA of a subject can be modified in vivo or ex vivo. In one embodiment, where the mtDNA is modified ex vivo, after modification the cells containing the modified mitochondria can be administered back to the subject. In one embodiment, the composition, system, or component thereof can be capable of correcting an mtDNA mutation, or a combination thereof.

In one embodiment, at least one of the one or more mtDNA mutations is selected from the group consisting of: A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC-tandem (SEQ ID NO: 1566) repeats at positions 305-314 and/or 956-965, deletion at positions from 8,469-13,447, 4,308-14,874, and/or 4,398-14,822, 961ins/delC, the mitochondrial common deletion (e.g. mtDNA 4,977 bp deletion), and combinations thereof.

In one embodiment, the mitochondrial mutation can be any mutation as set forth in or as identified by use of one or more bioinformatic tools available at Mitomap available at mitomap.org. Such tools include, but are not limited to, “Variant Search, aka Market Finder”, Find Sequences for Any Haplogroup, aka “Sequence Finder”, “Variant Info”, “POLG Pathogenicity Prediction Server”, “MITOMASTER”, “Allele Search”, “Sequence and Variant Downloads”, “Data Downloads”. MitoMap contains reports of mutations in mtDNA that can be associated with disease and maintains a database of reported mitochondrial DNA Base Substitution Diseases: rRNA/tRNA mutations.

In one embodiment, the method includes delivering a composition, system, and/or a component thereof to a cell, and more specifically one or more mitochondria in a cell, allowing the composition, system, and/or component thereof to modify one or more target polynucleotides in the cell, and more specifically one or more mitochondria in the cell. The target polynucleotides can correspond to a mutation in the mtDNA, such as any one or more of those described herein. In one embodiment, the modification can alter a function of the mitochondria such that the mitochondria functions normally or at least is/are less dysfunctional as compared to an unmodified mitochondria. Modification can occur in vivo or ex vivo. Where modification is performed ex vivo, cells containing modified mitochondria can be administered to a subject in need thereof in an autologous or allogenic manner.

### Microbiome Modification

Microbiomes play important roles in health and disease. For example, the gut microbiome can play a role in health by controlling digestion, preventing growth of pathogenic microorganisms and have been suggested to influence mood and emotion. Imbalanced microbiomes can promote disease and are suggested to contribute to weight gain, unregulated blood sugar, high cholesterol, cancer, and other disorders. A healthy microbiome has a series of joint characteristics that can be distinguished from non-healthy individuals, thus detection and identification of the disease-associated microbiome can be used to diagnose and detect disease in an individual. The compositions, systems, and components thereof can be used to screen the microbiome cell population and be used to identify a disease associated microbiome. Cell screening methods utilizing compositions, systems, and components thereof are described elsewhere herein and can be applied to screening a microbiome, such as a gut, skin, vagina, and/or oral microbiome, of a subject.

In one embodiment, the microbe population of a microbiome in a subject can be modified using a composition, system, and/or component thereof described herein. In one embodiment, the composition, system, and/or component thereof can be used to identify and select one or more cell types in the microbiome and remove them from the microbiome population. Exemplary methods of selecting cells using a composition, system, and/or component thereof are described elsewhere herein. In this way the make-up or microorganism profile of the microbiome can be altered. In one embodiment, the alteration causes a change from a diseased microbiome composition to a healthy microbiome composition. In this way the ratio of one type or species of microorganism to another can be modified, such as going from a diseased ratio to a healthy ratio. In one embodiment, the cells selected are pathogenic microorganisms.

In one embodiment, the compositions and systems described herein can be used to modify a polynucleotide in a microorganism of a microbiome in a subject. In one embodiment, the microorganism is a pathogenic microorganism. In one embodiment, the microorganism is a commensal and non-pathogenic microorganism. Methods of modifying polynucleotides in a cell in the subject are described elsewhere herein and can be applied to these embodiments.

### Models of Diseases and Conditions

In an aspect, the invention provides a method of modeling a disease associated with a genomic locus in a eukaryotic organism or a non-human organism comprising manipulation of a target sequence within a coding, non-coding or regulatory element of said genomic locus comprising delivering a non-naturally occurring or engineered composition comprising a viral vector system comprising one or more viral vectors operably encoding a composition for expression thereof, wherein the composition comprises particle delivery system or the delivery system or the virus particle of any one of the above embodiments or the cell of any one of the above embodiment.

In one aspect, the invention provides a method of generating a model eukaryotic cell that can include one or more a mutated disease genes and/or infectious microorganisms. In one embodiment, a disease gene is any gene associated an increase in the risk of having or developing a disease. In one embodiment, the method includes (a) introducing one or more vectors into a eukaryotic cell, wherein the one or more vectors comprise a composition, system, and/or component thereof and/or a vector or vector system that is capable of driving expression of a composition, system, and/or component thereof including, but not limited to: a ωRNA sequence, one or more IsrB polypeptide nucleases, and combinations thereof and (b) allowing a composition, system, or complex to bind to one or more target polynucleotides, e.g., to effect cleavage, nicking, or other modification of the target polynucleotide within said disease gene, wherein the composition, system, or complex is composed of one or more IsrB polypeptide nuclease complexed with (1) one or more ωRNAs that is/are hybridized to the target sequence(s) within the target polynucleotide(s), and optionally (2) the ωRNA scaffold sequence(s), thereby generating a model eukaryotic cell comprising one or more mutated disease gene(s). Thus, In one embodiment the composition and system, contains nucleic acid molecules for and drives expression of one or more of: a IsrB polypeptide nuclease, a ωRNA sequence and/or a Homologous Recombination template and/or a stabilizing ligand if the IsrB polypeptide nuclease has a destabilization domain. In one embodiment, said cleavage comprises cleaving one or two strands at the location of the target sequence by the IsrB polypeptide nuclease. In one embodiment, nicking comprises nicking one or two strands at the location of the target sequence by the IsrB polypeptide nuclease. In one embodiment, said cleavage or nicking results in modified transcription of a target polynucleotide. In one embodiment, modification results in decreased transcription of the target polynucleotide. In one embodiment, the method further comprises repairing said cleaved or nicked target polynucleotide by homologous recombination with a recombination template polynucleotide, wherein said repair results in a mutation comprising an insertion, deletion, or substitution of one or more nucleotides of said target polynucleotide. In one embodiment, said mutation results in one or more amino acid changes in a protein expression from a gene comprising the target sequence.

The disease modeled can be any disease with a genetic or epigenetic component. In one embodiment, the disease modeled can be any as discussed elsewhere herein.

### In Situ Disease Detection

The compositions, systems, and/or components thereof can be used for diagnostic methods of detection such as in CASFISH (see e.g. Deng et al. 2015. PNAS USA 112 (38): 11870-11875), CRISPR-Live FISH (see e.g. Wang et al. 2020. Science; 365 (6459): 1301-1305), sm-FISH (Lee and Jefcoate. 2017. Front. Endocrinol. doi.org/10.3389/fendo.2017.00289), sequential FISH CRISPRainbow (Ma et al. Nat Biotechnol, 34 (2016), pp. 528-530), CRISPR-Sirius (Nat Methods, 15 (2018), pp. 928-931), Casilio (Cheng et al. Cell Res, 26 (2016), pp. 254-257), Halo-Tag based genomic loci visualization techniques (e.g. Deng et al. 2015. PNAS USA 112 (38): 11870-11875; Knight et al., Science, 350 (2015), pp. 823-826), RNA-aptamer based methods (e.g. Ma et al., J Cell Biol, 214 (2016), pp. 529-537), molecular beacon-based methods (e.g. Zhao et al. Biomaterials, 100 (2016), pp. 172-183; Wu et al. Nucleic Acids Res (2018)), Quantum Dot-based systems (e.g. Ma et al. Anal Chem, 89 (2017), pp. 12896-12901), multiplexed methods (e.g. Ma et al., Proc Natl Acad Sci USA, 112 (2015), pp. 3002-3007; Fu et al. Nat Commun, 7 (2016), p. 11707; Ma et al. Nat Biotechnol, 34 (2016), pp. 528-530; Shao et al. Nucleic Acids Res, 44 (2016), Article e86); Wang et al. Sci Rep, 6 (2016), p. 26857), ç, and other in situ CRISPR-hybridization based methods (e.g. Chen et al. Cell, 155 (2013), pp. 1479-1491; Gu et al. Science, 359 (2018), pp. 1050-1055; Tanebaum et al. Cell, 159 (2014), pp. 635-646; Ye et al. Protein Cell, 8 (2017), pp. 853-855; Chen et al. Nat Commun, 9 (2018), p. 5065; Shao et al. ACS Synth Biol (2017); Fu et al. Nat Commun, 7 (2016), p. 11707; Shao et al. Nucleic Acids Res, 44 (2016), Article e86; Wang et al., Sci Rep, 6 (2016), p. 26857), all of which are incorporated by reference herein as if expressed in their entirety and whose teachings can be adapted to the compositions, systems, and components thereof described herein in view of the description herein.

In one embodiment, the composition, system, or component thereof can be used in a detection method, such as an in-situ detection method described herein. In one embodiment, the composition, system, or component thereof can include a catalytically inactivate IscB polypeptide nuclease described herein and use this system in detection methods such as fluorescence in situ hybridization (FISH) or any other described herein. In one embodiment, the inactivated IscB polypeptide nuclease, which lacks the ability to produce DNA double-strand breaks may be fused with a marker, such as fluorescent protein, such as the enhanced green fluorescent protein (eEGFP) and co-expressed with small guide RNAs to target pericentric, centric and telomeric repeats in vivo. The dead IsrB polypeptide nuclease or system thereof can be used to visualize both repetitive sequences and individual genes in the human genome. Such new applications of labelled dead IsrB polypeptide nuclease and compositions, systems, thereof can be important in imaging cells and studying the functional nuclear architecture, especially in cases with a small nucleus volume or complex 3-D structures.

### Cell Selection

In one embodiment, the compositions, systems, and/or components thereof described herein can be used in a method to screen and/or select cells. In one embodiment, composition, system,-based screening/selection method can be used to identify diseased cells in a cell population. In one embodiment, selection of the cells results in a modification in the cells such that the selected cells die. In this way, diseased cells can be identified, and removed from the healthy cell population. In one embodiment, the diseased cells can be a cancer cell, pre-cancerous cell, a virus or other pathogenic organism infected cells, or otherwise abnormal cell. In one embodiment, the modification can impart another detectable change in the cells to be selected (e.g. a functional change and/or genomic barcode) that facilitates selection of the desired cells. In one embodiment a negative selection scheme can be used to obtain a desired cell population. In these embodiments, the cells to be selected against are modified, thus can be removed from the cell population based on their death or identification or sorting based the detectable change imparted on the cells. Thus, in these embodiments, the remaining cells after selection are the desired cell population.

In one embodiment, a method of selecting one or more cell(s) containing a polynucleotide modification can include: introducing one or more composition, system, (s) and/or components thereof, and/or vectors or vector systems into the cell(s), wherein the composition, system, (s) and/or components thereof, and/or vectors or vector systems contains and/or is capable of expressing one or more of: a IsrB polypeptide nuclease, an ωRNA sequence, and an recombination template; wherein, for example that which is being expressed is within and expressed in vivo by the composition, system, vector or vector system and/or the recombination template comprises the one or more mutations that abolish IsrB polypeptide nuclease nicking or cleavage; allowing homologous recombination of the recombination template with the target polynucleotide in the cell(s) to be selected; allowing a composition, system, or complex to bind to a target polynucleotide to effect cleavage of the target polynucleotide within said gene, wherein the AAV-complex comprises the IsrB polypeptide nuclease complexed with (1) the ωRNA that is hybridized to the target sequence within the target polynucleotide, and (2) the ωRNA scaffold, wherein binding of the complex to the target polynucleotide induces cell death or imparts some other detectable change to the cell, thereby allowing one or more cell(s) in which one or more mutations have been introduced to be selected. In one embodiment, the cell to be selected may be a eukaryotic cell. In one embodiment, the cell to be selected may be a prokaryotic cell. Selection of specific cells via the methods herein can be performed without requiring a selection marker or a two-step process that may include a counter-selection system.

### Therapeutic Agent Development

The compositions, systems, and components thereof described herein can be used to develop IscB polypeptide nuclease-based biologically active agents, such as small molecule therapeutics. Thus, described herein are methods for developing a biologically active agent that modulates a cell function and/or signaling event associated with a disease and/or disease gene. In one embodiment, the method comprises (a) contacting a test compound with a diseased cell and/or a cell containing a disease gene cell; and (b) detecting a change in a readout that is indicative of a reduction or an augmentation of a cell signaling event or other cell functionality associated with said disease or disease gene, thereby developing said biologically active agent that modulates said cell signaling event or other functionality associated with said disease gene. In one embodiment, the diseased cell is a model cell described elsewhere herein. In one embodiment, the diseased cell is a diseased cell isolated from a subject in need of treatment. In one embodiment, the test compound is a small molecule agent. In one embodiment, test compound is a small molecule agent. In one embodiment, the test compound is a biologic molecule agent.

In one embodiment, the method involves developing a therapeutic based on the composition, system, described herein. In one embodiment, the therapeutic comprises a IsrB polypeptide nuclease and/or a ωRNA with a reprogrammable spacer capable of hybridizing to a target sequence of interest. In one embodiment, the therapeutic is a vector or vector system that can contain a) a first regulatory element operably linked to a nucleotide sequence encoding the IsrB polypeptide nuclease; and b) a second regulatory element operably linked to one or more nucleotide sequences encoding one or more nucleic acid molecules comprising a ωRNA comprising a reprogrammable spacer sequence, a conserved RNA sequence; wherein components (a) and (b) are located on same or different vectors. In one embodiment, the biologically active agent is a composition comprising a delivery system operably configured to deliver composition, system, or components thereof, and/or or one or more polynucleotide sequences, vectors, or vector systems containing or encoding said components into a cell and capable of forming a complex with the components of the composition and system herein, and wherein said complex is operable in the cell. In one embodiment, the complex can include the IsrB polypeptide nuclease as described herein, ωRNA scaffold comprising the guide sequence (reprogrammable spacer sequence), and a conserved nucleotide sequence. In any such compositions, the delivery system can be a yeast system, a lipofection system, a microinjection system, a biolistic system, virosomes, liposomes, immunoliposomes, polycations, lipid: nucleic acid conjugates or artificial virions, or any other system as described herein. In one embodiment, the delivery is via a particle, a nanoparticle, a lipid or a cell penetrating peptide (CPP).

Also described herein are methods for developing or designing a composition, system, optionally a composition, system, based therapy or therapeutic, comprising (a) selecting for a (therapeutic) locus of interest ωRNA target sites, wherein said target sites have minimal sequence variation across a population, and from said selected target sites subselecting target sites, wherein a ωRNA directed against said target sites recognizes a minimal number of off-target sites across said population, or (b) selecting for a (therapeutic) locus of interest ωRNA target sites, wherein said target sites have minimal sequence variation across a population, or selecting for a (therapeutic) locus of interest ωRNA target sites, wherein a ωRNA directed against said target sites recognizes a minimal number of off-target sites across said population, and optionally estimating the number of (sub) selected target sites needed to treat or otherwise modulate or manipulate a population, and optionally validating one or more of the (sub) selected target sites for an individual subject, optionally designing one or more ωRNA recognizing one or more of said (sub) selected target sites.

In one embodiment, the method for developing or designing a ωRNA for use in a composition, system, optionally a composition, system, based therapy or therapeutic, can include (a) selecting for a (therapeutic) locus of interest ωRNA target sites, wherein said target sites have minimal sequence variation across a population, and from said selected target sites subselecting target sites, wherein a ωRNA directed against said target sites recognizes a minimal number of off-target sites across said population, or (b) selecting for a (therapeutic) locus of interest ωRNA target sites, wherein said target sites have minimal sequence variation across a population, or selecting for a (therapeutic) locus of interest ωRNA target sites, wherein a ωRNA directed against said target sites recognizes a minimal number of off-target sites across said population, and optionally estimating the number of (sub) selected target sites needed to treat or otherwise modulate or manipulate a population, optionally validating one or more of the (sub) selected target sites for an individual subject, optionally designing one or more ωRNA recognizing one or more of said (sub) selected target sites.

In one embodiment, thee method for developing or designing a composition, system, optionally a composition, system, based therapy or therapeutic in a population, can include (a) selecting for a (therapeutic) locus of interest reprogrammable spacer target sites, wherein said target sites have minimal sequence variation across a population, and from said selected target sites subselecting target sites, wherein a ωRNA directed against said target sites recognizes a minimal number of off-target sites across said population, or (b) selecting for a (therapeutic) locus of interest @ RNA reprogrammable spacer target sites, wherein said target sites have minimal sequence variation across a population, or selecting for a (therapeutic) locus of interest ωRNA reprogrammable spacer target sites, wherein a ωRNA directed against said target sites recognizes a minimal number of off-target sites across said population, and optionally estimating the number of (sub) selected target sites needed to treat or otherwise modulate or manipulate a population, optionally validating one or more of the (sub) selected target sites for an individual subject, optionally designing one or more ωRNA recognizing one or more of said (sub) selected target sites.

In one embodiment the method for developing or designing a ωRNA for use in a composition, system, optionally a composition, system, based therapy or therapeutic in a population, can include (a) selecting for a (therapeutic) locus of interest ωRNA target sites, wherein said target sites have minimal sequence variation across a population, and from said selected target sites subselecting target sites, wherein a ωRNA directed against said target sites recognizes a minimal number of off-target sites across said population, or (b) selecting for a (therapeutic) locus of interest ωRNA target sites, wherein said target sites have minimal sequence variation across a population, or selecting for a (therapeutic) locus of interest ωRNA target sites, wherein a ωRNA directed against said target sites recognizes a minimal number of off-target sites across said population, and optionally estimating the number of (sub) selected target sites needed to treat or otherwise modulate or manipulate a population, optionally validating one or more of the (sub) selected target sites for an individual subject, optionally designing one or more ωRNA reprogrammable spacer recognizing one or more of said (sub) selected target sites.

In one embodiment, the method for developing or designing a composition, system, such as a composition, system, based therapy or therapeutic, optionally in a population; or for developing or designing a ωRNA reprogrammable spacer for use in a composition, system, optionally a composition, system, based therapy or therapeutic, optionally in a population, can include selecting a set of target sequences for one or more loci in a target population, wherein the target sequences do not contain variants occurring above a threshold allele frequency in the target population (i.e. platinum target sequences); removing from said selected (platinum) target sequences any target sequences having high frequency off-target candidates (relative to other (platinum) targets in the set) to define a final target sequence set; preparing one or more, such as a set of compositions, systems, based on the final target sequence set, optionally wherein a number of composition prepared is based (at least in part) on the size of a target population.

In an embodiment, off-target candidates/off-targets, TAM restrictiveness, target cleavage efficiency, or effector protein specificity is identified or determined using a sequencing-based double-strand break (DSB) detection assay, such as described herein elsewhere. In an embodiment, off-target candidates/off-targets are identified or determined using a sequencing-based double-strand break (DSB) detection assay, such as described herein elsewhere. In an embodiment, off-targets, or off target candidates have at least 1, preferably 1-3, mismatches or (distal) TAM mismatches, such as 1 or more, such as 1, 2, 3, or more (distal) TAM mismatches. In an embodiment, sequencing-based DSB detection assay comprises labeling a site of a DSB with an adapter comprising a primer binding site, labeling a site of a DSB with a barcode or unique molecular identifier, or combination thereof, as described herein elsewhere.

It will be understood that the reprogrammable spacer sequence of the ωRNA is 100% complementary to the target site, i.e. does not comprise any mismatch with the target site. It will be further understood that “recognition” of an (off-) target site by a reprogrammable spacer presupposes composition, system, functionality, i.e. an (off-) target site is only recognized by a reprogrammable spacer RNA if binding of the reprogrammable spacer RNA to the (off-) target site leads to composition, system, activity (such as induction of single or double strand DNA cleavage, transcriptional modulation, etc.).

In an embodiment, the target sites having minimal sequence variation across a population are characterized by absence of sequence variation in at least 99%, preferably at least 99.9%, more preferably at least 99.99% of the population. In an embodiment, optimizing target location comprises selecting target sequences or loci having an absence of sequence variation in at least 99%, %, preferably at least 99.9%, more preferably at least 99.99% of a population. These targets are referred to herein elsewhere also as “platinum targets”. In an embodiment, said population comprises at least 1000 individuals, such as at least 5000 individuals, such as at least 10000 individuals, such as at least 50000 individuals.

In an embodiment, the off-target sites are characterized by at least one mismatch between the off-target site and the ωRNA. In an embodiment, the off-target sites are characterized by at most five, preferably at most four, more preferably at most three mismatches between the off-target site and the ωRNA. In an embodiment, the off-target sites are characterized by at least one mismatch between the off-target site and the ωRNA and by at most five, preferably at most four, more preferably at most three mismatches between the off-target site and the ωRNA.

In an embodiment, said minimal number of off-target sites across said population is determined for high-frequency haplotypes in said population. In an embodiment, said minimal number of off-target sites across said population is determined for high-frequency haplotypes of the off-target site locus in said population. In an embodiment, said minimal number of off-target sites across said population is determined for high-frequency haplotypes of the target site locus in said population. In an embodiment, the high-frequency haplotypes are characterized by occurrence in at least 0.1% of the population.

In an embodiment, the number of (sub) selected target sites needed to treat a population is estimated based on based low frequency sequence variation, such as low frequency sequence variation captured in large scale sequencing datasets. In an embodiment, the number of (sub) selected target sites needed to treat a population of a given size is estimated.

In an embodiment, the method further comprises obtaining genome sequencing data of a subject to be treated; and treating the subject with a composition, system, selected from the set of compositions, systems, wherein the composition, system, selected is based (at least in part) on the genome sequencing data of the individual. In an embodiment, the ((sub) selected) target is validated by genome sequencing, preferably whole genome sequencing.

In an embodiment, target sequences or loci as described herein are (further) selected based on optimization of one or more parameters, such as TAM type (natural or modified), TAM nucleotide content, TAM length, target sequence length, TAM restrictiveness, target cleavage efficiency, and target sequence position within a gene, a locus or other genomic region. Methods of optimization are discussed in greater detail elsewhere herein.

In an embodiment, target sequences or loci as described herein are (further) selected based on optimization of one or more of target loci location, target length, target specificity, and TAM characteristics. As used herein, TAM characteristics may comprise for instance TAM sequence, TAM length, and/or TAM GC contents. In an embodiment, optimizing TAM characteristics comprises optimizing nucleotide content of a TAM. In an embodiment, optimizing nucleotide content of TAM is selecting a TAM with a motif that maximizes abundance in the one or more target loci, minimizes mutation frequency, or both. Minimizing mutation frequency can for instance be achieved by selecting TAM sequences devoid of or having low or minimal CpG.

In an embodiment, the effector protein for each composition and system, in the set of compositions, systems, is selected based on optimization of one or more parameters selected from the group consisting of; effector protein size, ability of effector protein to access regions of high chromatin accessibility, degree of uniform enzyme activity across genomic targets, epigenetic tolerance, mismatch/budge tolerance, effector protein specificity, effector protein stability or half-life, effector protein immunogenicity or toxicity. Methods of optimization are discussed in greater detail elsewhere herein.

### Optimization of the Systems

The methods of the present invention can involve optimization of selected parameters or variables associated with the composition, system, and/or its functionality, as described herein further elsewhere. Optimization of the composition, system, in the methods as described herein may depend on the target(s), such as the therapeutic target or therapeutic targets, the mode or type of composition, system, modulation, such as composition, system, based therapeutic target(s) modulation, modification, or manipulation, as well as the delivery of the composition, system, components. One or more targets may be selected, depending on the genotypic and/or phenotypic outcome. For instance, one or more therapeutic targets may be selected, depending on (genetic) disease etiology or the desired therapeutic outcome. The (therapeutic) target(s) may be a single gene, locus, or other genomic site, or may be multiple genes, loci or other genomic sites. As is known in the art, a single gene, locus, or other genomic site may be targeted more than once, such as by use of multiple ωRNAs, or ωRNA scaffold and multiple reprogrammable spacers.

The activity of the composition and/or system, such as IsrB polypeptide nuclease-based therapy or therapeutics may involve target disruption, such as target mutation, such as leading to gene knockout. The activity of the composition and/or system, such as IscB polypeptide nuclease-based therapy or therapeutics may involve replacement of particular target sites, such as leading to target correction. IsrB polypeptide nuclease-based therapy or therapeutics may involve removal of particular target sites, such as leading to target deletion. The activity of the composition and/or system, such as IsrB polypeptide nuclease-based therapy or therapeutics may involve modulation of target site functionality, such as target site activity or accessibility, leading for instance to (transcriptional and/or epigenetic) gene or genomic region activation or gene or genomic region silencing. The skilled person will understand that modulation of target site functionality may involve IsrB polypeptide nuclease mutation (such as for instance generation of a catalytically inactive IsrB polypeptide nuclease) and/or functionalization (such as for instance fusion of the IsrB polypeptide nuclease with a heterologous functional domain, such as a transcriptional activator or repressor), as described herein elsewhere.

Accordingly, in an aspect, the invention relates to a method as described herein, comprising selection of one or more (therapeutic) target, selecting one or more functionality of the composition and/or system, and optimization of selected parameters or variables associated with the composition and/or its functionality. In a related aspect, the invention relates to a method as described herein, comprising (a) selecting one or more (therapeutic) target loci, (b) selecting one or more composition functionalities, (c) optionally selecting one or more modes of delivery, and preparing, developing, or designing a composition herein selected based on steps (a)-(c).

In an embodiment, the functionality of the composition and/or system comprises genomic mutation. In an embodiment, the functionality of the composition and/or system comprises single genomic mutation. In an embodiment, the functionality of the composition and/or system functionality comprises multiple genomic mutation. In an embodiment, the functionality of the composition and/or system comprises gene knockout. In an embodiment, the functionality of the composition and/or system comprises single gene knockout. In an embodiment, the functionality of the composition and/or system comprises multiple gene knockout. In an embodiment, the functionality of the composition and/or system comprises gene correction. In an embodiment, the functionality of the composition and/or system comprises single gene correction. In an embodiment, the functionality of the composition and/or system comprises multiple gene correction. In an embodiment, the functionality of the composition and/or system comprises genomic region correction. In an embodiment, the functionality of the composition and/or system comprises single genomic region correction. In an embodiment, the functionality of the composition and/or system comprises multiple genomic region correction. In an embodiment, the functionality of the composition and/or system comprises gene deletion. In an embodiment, the functionality of the composition and/or system comprises single gene deletion. In an embodiment, the functionality of the composition and/or system comprises multiple gene deletion. In an embodiment, the functionality of the composition and/or system comprises genomic region deletion. In an embodiment, the functionality of the composition and/or system comprises single genomic region deletion. In an embodiment, the functionality of the composition and/or system comprises multiple genomic region deletion. In an embodiment, the functionality of the composition and/or system comprises modulation of gene or genomic region functionality. In an embodiment, the functionality of the composition and/or system comprises modulation of single gene or genomic region functionality. In an embodiment, the functionality of the composition and/or system comprises modulation of multiple gene or genomic region functionality. In an embodiment, the functionality of the composition and/or system comprises gene or genomic region functionality, such as gene or genomic region activity. In an embodiment, the functionality of the composition and/or system comprises single gene or genomic region functionality, such as gene or genomic region activity. In an embodiment, the functionality of the composition and/or system comprises multiple gene or genomic region functionality, such as gene or genomic region activity. In an embodiment, the functionality of the composition and/or system comprises modulation gene activity or accessibility optionally leading to transcriptional and/or epigenetic gene or genomic region activation or gene or genomic region silencing. In an embodiment, the functionality of the composition and/or system comprises modulation single gene activity or accessibility optionally leading to transcriptional and/or epigenetic gene or genomic region activation or gene or genomic region silencing. In an embodiment, the functionality of the composition and/or system comprises modulation multiple gene activity or accessibility optionally leading to transcriptional and/or epigenetic gene or genomic region activation or gene or genomic region silencing.

Optimization of selected parameters or variables in the methods as described herein may result in optimized or improved the system, such as IsrB polypeptide nuclease-based therapy or therapeutic, specificity, efficacy, and/or safety. In an embodiment, one or more of the following parameters or variables are taken into account, are selected, or are optimized in the methods of the invention as described herein: IsrB polypeptide nuclease allosteric interactions, IsrB polypeptide nuclease functional domains and functional domain interactions, IsrB polypeptide nuclease specificity, hRNA specificity, composition specificity, TAM restrictiveness, TAM type (natural or modified), TAM nucleotide content, TAM length, IsrB polypeptide nuclease activity, hRNA activity, IsrB polypeptide nuclease/guide complex activity, target cleavage efficiency, target site selection, target sequence length, ability of effector protein to access regions of high chromatin accessibility, degree of uniform enzyme activity across genomic targets, epigenetic tolerance, mismatch/budge tolerance, IsrB polypeptide nuclease stability, IsrB polypeptide nuclease mRNA stability, ωRNA stability, IsrB polypeptide nuclease complex stability, IsrB polypeptide nuclease protein or mRNA immunogenicity or toxicity, ωRNA immunogenicity or toxicity, IsrB polypeptide nuclease immunogenicity or toxicity, IsrB polypeptide nuclease or mRNA dose or titer, ωRNA dose or titer, dose or titer, IsrB polypeptide nuclease protein size, IsrB polypeptide nuclease expression level, ωRNA expression level, IsrB polypeptide nuclease expression level, IsrB polypeptide nuclease spatiotemporal expression, ωRNA spatiotemporal expression, IsrB polypeptide nuclease/hRNA spatiotemporal expression.

By means of example, and without limitation, parameter or variable optimization may be achieved as follows. IsrB polypeptide nuclease specificity may be optimized by selecting the most specific IsrB polypeptide nuclease, e.g. IsrB polypeptide. This may be achieved for instance by selecting the most specific IsrB polypeptide nuclease orthologue or by specific IsrB polypeptide nuclease mutations which increase specificity. ωRNA specificity may be optimized by selecting the most specific ωRNA. This can be achieved for instance by selecting ωRNA having low homology, i.e. at least one or preferably more, such as at least 2, or preferably at least 3, mismatches to off-target sites. The specificity may be optimized by increasing IsrB polypeptide nuclease specificity and/or ωRNA specificity as above. TAM restrictiveness may be optimized by selecting a IsrB polypeptide nuclease having to most restrictive TAM recognition. This can be achieved for instance by selecting a IsrB polypeptide nuclease ortholog having more restrictive TAM recognition or by specific IsrB polypeptide mutations which increase or alter TAM restrictiveness. TAM type may be optimized for instance by selecting the appropriate IsrB polypeptide nuclease, such as the appropriate IsrB polypeptide nuclease recognizing a desired TAM type. The IsrB polypeptide nuclease or TAM type may be naturally occurring or may for instance be optimized based on IsrB polypeptide nuclease mutants having an altered TAM recognition, or TAM recognition repertoire. TAM nucleotide content may for instance be optimized by selecting the appropriate IsrB polypeptide nuclease, such as the appropriate IsrB nuclease recognizing a desired TAM nucleotide content. The IsrB polypeptide nuclease or TAM type may be naturally occurring or may for instance be optimized based on IsrB polypeptide nuclease mutants having an altered TAM recognition, or TAM recognition repertoire. TAM length may for instance be optimized by selecting the appropriate IsrB polypeptide nuclease, such as the appropriate IsrB polypeptide nuclease recognizing a desired TAM nucleotide length. The IsrB polypeptide nuclease or TAM type may be naturally occurring or may for instance be optimized based on IsrB polypeptide nuclease mutants having an altered TAM recognition, or TAM recognition repertoire.

Target length or target sequence length may be optimized, for instance, by selecting the appropriate IsrB polypeptide nuclease, such as the appropriate IsrB polypeptide nuclease recognizing a desired target or target sequence nucleotide length. Alternatively, or in addition, the target (sequence) length may be optimized by providing a target having a length deviating from the target (sequence) length typically associated with the IsrB polypeptide nuclease, such as the naturally occurring IsrB polypeptide nuclease. The IsrB polypeptide nuclease or target (sequence) length may be naturally occurring or may for instance be optimized based on IsrB polypeptide nuclease mutants having an altered target (sequence) length recognition, or target (sequence) length recognition repertoire. For instance, increasing or decreasing target (sequence) length may influence target recognition and/or off-target recognition. IsrB polypeptide nuclease activity may be optimized by selecting the most active IsrB polypeptide nuclease. This may be achieved for instance by selecting the most active IsrB polypeptide nuclease ortholog or by specific IsrB polypeptide nuclease mutations which increase activity. The ability of the IsrB polypeptide nuclease protein to access regions of high chromatin accessibility, may be optimized by selecting the appropriate IsrB polypeptide nuclease or mutant thereof, and can consider the size of the IsrB polypeptide nuclease, charge, or other dimensional variables etc. The degree of uniform IsrB polypeptide nuclease activity may be optimized by selecting the appropriate IsrB polypeptide nuclease or mutant thereof, and can consider IsrB polypeptide nuclease specificity and/or activity, TAM specificity, target length, mismatch tolerance, epigenetic tolerance, IsrB polypeptide nuclease and/or ωRNA stability and/or half-life, IsrB polypeptide nuclease and/or ωRNA immunogenicity and/or toxicity, etc. ωRNA activity may be optimized by selecting the most active @ RNA. In one embodiment, this can be achieved by increasing ωRNA stability through RNA modification. compositions activity may be optimized by increasing IsrB polypeptide nuclease activity and/or ωRNA activity as above.

The target site selection may be optimized by selecting the optimal position of the target site within a gene, locus or other genomic region. The target site selection may be optimized by optimizing target location comprises selecting a target sequence with a gene, locus, or other genomic region having low variability. This may be achieved for instance by selecting a target site in an early and/or conserved exon or domain (i.e. having low variability, such as polymorphisms, within a population).

In an embodiment, optimizing target (sequence) length comprises selecting a target sequence within one or more target loci between 5 and 25 nucleotides. In an embodiment, a target sequence is 20 nucleotides.

In an embodiment, optimizing target specificity comprises selecting targets loci that minimize off-target candidates.

In one embodiment, the target site may be selected by minimization of off-target effects (e.g. off-targets qualified as having 1-5, 1-4, or preferably 1-3 mismatches compared to target and/or having one or more TAM mismatches, such as distal TAM mismatches), preferably also considering variability within a population. IsrB polypeptide nuclease stability may be optimized by selecting IsrB polypeptide nuclease having appropriate half-life, such as preferably a short half-life while still capable of maintaining sufficient activity. In one embodiment, this can be achieved by selecting an appropriate IsrB polypeptide nuclease orthologue having a specific half-life or by specific IsrB polypeptide nuclease mutations or modifications which affect half-life or stability, such as inclusion (e.g. fusion) of stabilizing or destabilizing domains or sequences. IsrB polypeptide nuclease mRNA stability may be optimized by increasing or decreasing IsrB polypeptide nuclease mRNA stability. In one embodiment, this can be achieved by increasing or IsrB polypeptide nuclease mRNA stability through mRNA modification. hRNA stability may be optimized by increasing or decreasing ωRNA stability. In one embodiment, this can be achieved by increasing or decreasing ωRNA stability through RNA modification. The stability may be optimized by increasing or decreasing IsrB polypeptide nuclease stability and/or ωRNA stability as above. IsrB polypeptide nuclease protein or mRNA immunogenicity or toxicity may be optimized by decreasing IsrB polypeptide nuclease or mRNA immunogenicity or toxicity. In one embodiment, this can be achieved by mRNA or protein modifications. Similarly, in case of DNA based expression systems, DNA immunogenicity or toxicity may be decreased. ωRNA immunogenicity or toxicity may be optimized by decreasing hRNA immunogenicity or toxicity. In one embodiment, this can be achieved by ωRNA modifications. Similarly, in case of DNA based expression systems, DNA immunogenicity or toxicity may be decreased. The immunogenicity or toxicity may be optimized by decreasing IsrB polypeptide nuclease immunogenicity or toxicity and/or @ RNA immunogenicity or toxicity as above, or by selecting the least immunogenic or toxic IsrB polypeptide nuclease/hRNA combination. Similarly, in case of DNA based expression systems, DNA immunogenicity or toxicity may be decreased. IsrB polypeptide nuclease protein or mRNA dose or titer may be optimized by selecting dosage or titer to minimize toxicity and/or maximize specificity and/or efficacy. ωRNA dose or titer may be optimized by selecting dosage or titer to minimize toxicity and/or maximize specificity and/or efficacy. The composition dose or titer may be optimized by selecting dosage or titer to minimize toxicity and/or maximize specificity and/or efficacy. The IsrB polypeptide nuclease size may be optimized by selecting minimal protein size to increase efficiency of delivery, in particular for virus mediated delivery. IsrB polypeptide nuclease, ωRNA, or complex thereof expression level may be optimized by limiting (or extending) the duration of expression and/or limiting (or increasing) expression level. This may be achieved for instance by using self-inactivating compositions, systems, such as including a self-targeting (e.g. IsrB polypeptide nuclease targeting) ωRNA, by using viral vectors having limited expression duration, by using appropriate promoters for low (or high) expression levels, by combining different delivery methods for individual IsrB polypeptide system components, such as virus mediated delivery of IsrB polypeptide nuclease encoding nucleic acid combined with non-virus mediated delivery of ωRNA, or virus mediated delivery of ωRNA combined with non-virus mediated delivery of IsrB polypeptide nuclease or mRNA. IsrB polypeptide nuclease, ωRNA, or IsrB polypeptide complex spatiotemporal expression may be optimized by appropriate choice of conditional and/or inducible expression systems, including controllable IsrB polypeptide nuclease activity optionally a destabilized IsrB polypeptide nuclease and/or a split IsrB polypeptide nuclease, and/or cell- or tissue-specific expression systems.

In an aspect, the invention relates to a method as described herein, comprising selection of one or more (therapeutic) target, selecting the functionality of the composition and/or system, selecting composition mode of delivery, selecting composition delivery vehicle or expression system, and optimization of selected parameters or variables associated with the composition and/or its functionality, optionally wherein the parameters or variables are one or more selected from IsrB polypeptide nuclease specificity, ωRNA specificity, IsrB polypeptide complex specificity, TAM restrictiveness, TAM type (natural or modified), TAM nucleotide content, TAM length, IsrB polypeptide nuclease activity, ωRNA activity, IsrB polypeptide nuclease/hRNA complex activity, target cleavage efficiency, target site selection, target sequence length, ability of effector protein to access regions of high chromatin accessibility, degree of uniform enzyme activity across genomic targets, epigenetic tolerance, mismatch/budge tolerance, IsrB polypeptide nuclease stability, IsrB polypeptide nuclease mRNA stability, ωRNA stability, IsrB polypeptide complex stability, IsrB polypeptide nuclease protein or mRNA immunogenicity or toxicity, ωRNA immunogenicity or toxicity, IsrB polypeptide nuclease/ωRNA complex immunogenicity or toxicity, IsrB polypeptide nuclease protein or mRNA dose or titer, ωRNA dose or titer, IsrB polypeptide complex dose or titer, IsrB polypeptide nuclease protein size, IsrB polypeptide nuclease expression level, ωRNA expression level, IsrB polypeptide nuclease/@ RNA molecule complex expression level, IsrB polypeptide nuclease spatiotemporal expression, ωRNA spatiotemporal expression, IsrB polypeptide nuclease/@ RNA complex spatiotemporal expression.

It will be understood that the parameters or variables to be optimized as well as the nature of optimization may depend on the (therapeutic) target, the functionality of the composition and/or system, the system mode of delivery, and/or the composition delivery vehicle or expression system.

In an aspect, the invention relates to a method as described herein, comprising optimization of ωRNA specificity at the population level. Preferably, said optimization of ωRNA specificity comprises minimizing ωRNA target site sequence variation across a population and/or minimizing ωRNA off-target incidence across a population.

In one embodiment, optimization can result in selection of a IsrB polypeptide nuclease that is naturally occurring or is modified. In one embodiment, optimization can result in selection of a IsrB polypeptide nuclease that has nuclease, nickase, deaminase, transposase, and/or has one or more effector functionalities deactivated or eliminated. In one embodiment, optimizing a TAM specificity can include selecting a IsrB polypeptide nuclease with a modified TAM specificity. In one embodiment, optimizing can include selecting a IsrB polypeptide nuclease having a minimal size. In an embodiment, optimizing effector protein stability comprises selecting an effector protein having a short half-life while maintaining sufficient activity, such as by selecting an appropriate IsrB polypeptide nuclease orthologue having a specific half-life or stability. In an embodiment, optimizing immunogenicity or toxicity comprises minimizing effector protein immunogenicity or toxicity by protein modifications. In an embodiment, optimizing functional specific comprises selecting a protein effector with reduced tolerance of mismatches and/or bulges between the ωRNA and one or more target loci.

In an embodiment, optimizing efficacy comprises optimizing overall efficiency, epigenetic tolerance, or both. In an embodiment, maximizing overall efficiency comprises selecting an effector protein with uniform enzyme activity across target loci with varying chromatin complexity, selecting an effector protein with enzyme activity limited to areas of open chromatin accessibility. In an embodiment, chromatin accessibility is measured using one or more of ATAC-seq, or a DNA-proximity ligation assay. In an embodiment, optimizing epigenetic tolerance comprises optimizing methylation tolerance, epigenetic mark competition, or both. In an embodiment, optimizing methylation tolerance comprises selecting an effector protein that modify methylated DNA. In an embodiment, optimizing epigenetic tolerance comprises selecting an effector protein unable to modify silenced regions of a chromosome, selecting an effector protein able to modify silenced regions of a chromosome, or selecting target loci not enriched for epigenetic markers

In an embodiment, selecting an optimized ωRNA comprises optimizing ωRNA stability, ωRNA immunogenicity, or both, or other ωRNA associated parameters or variables as described herein elsewhere.

In an embodiment, optimizing ωRNA stability and/or ωRNA immunogenicity comprises RNA modification, or other ωRNA associated parameters or variables as described herein elsewhere. In an embodiment, the modification comprises removing 1-3 nucleotides form the 3′ end of a target complementarity region of the ωRNA. In an embodiment, modification comprises an extended @ RNA and/or trans RNA/DNA element that create stable structures in the ωRNA that compete with ωRNA base pairing at a target of off-target loci, or extended complimentary nucleotides between the ωRNA and target sequence, or both.

In an embodiment, the mode of delivery comprises delivering ωRNA and/or IsrB polypeptide nuclease, delivering @ RNA and/or IsrB polypeptide nuclease mRNA, or delivery ωRNA and/or IsrB polypeptide nuclease as a DNA based expression system. In an embodiment, the mode of delivery further comprises selecting a delivery vehicle and/or expression systems from the group consisting of liposomes, lipid particles, nanoparticles, biolistics, or viral-based expression/delivery systems. In an embodiment, expression is spatiotemporal expression is optimized by choice of conditional and/or inducible expression systems, including controllable IsrB polypeptide nuclease activity optionally a destabilized IsrB polypeptide polypeptide and/or a split IsrB polypeptide nuclease, and/or cell- or tissue-specific expression system.

The methods as described herein may further involve selection of the mode of delivery. In an embodiment, @ RNA and/or IsrB polypeptide nuclease are or are to be delivered. In an embodiment, ωRNA and/or IsrB polypeptide nuclease mRNA are or are to be delivered. In an embodiment, ωRNA and/or IsrB polypeptide nuclease provided in a DNA-based expression system are or are to be delivered. In an embodiment, delivery of the individual system components comprises a combination of the above modes of delivery. In an embodiment, delivery comprises delivering ωRNA and/or IsrB polypeptide nuclease protein, delivering ωRNA and/or IsrB polypeptide nuclease mRNA, or delivering ωRNA and/or IsrB polypeptide nuclease as a DNA based expression system.

The methods as described herein may further involve selection of the composition delivery vehicle and/or expression system. Delivery vehicles and expression systems are described herein elsewhere. By means of example, delivery vehicles of nucleic acids and/or proteins include nanoparticles, liposomes, etc. Delivery vehicles for DNA, such as DNA-based expression systems include for instance biolistics, viral based vector systems (e.g. adenoviral, AAV, lentiviral), etc. the skilled person will understand that selection of the mode of delivery, as well as delivery vehicle or expression system may depend on for instance the cell or tissues to be targeted. In an embodiment, the delivery vehicle and/or expression system for delivering the compositions, systems, or components thereof comprises liposomes, lipid particles, nanoparticles, biolistics, or viral-based expression/delivery systems.

### Considerations for Therapeutic Applications

A consideration in genome editing therapy is the choice of sequence-specific nuclease, such as a variant of a IsrB polypeptide nuclease. Each nuclease variant may possess its own unique set of strengths and weaknesses, many of which must be balanced in the context of treatment to maximize therapeutic benefit. For a specific editing therapy to be efficacious, a sufficiently high level of modification must be achieved in target cell populations to reverse disease symptoms. This therapeutic modification ‘threshold’ is determined by the fitness of edited cells following treatment and the amount of gene product necessary to reverse symptoms. With regard to fitness, editing creates three potential outcomes for treated cells relative to their unedited counterparts: increased, neutral, or decreased fitness. In the case of increased fitness, corrected cells may be able and expand relative to their diseased counterparts to mediate therapy. In this case, where edited cells possess a selective advantage, even low numbers of edited cells can be amplified through expansion, providing a therapeutic benefit to the patient. Where the edited cells possess no change in fitness, an increase the therapeutic modification threshold can be warranted. As such, significantly greater levels of editing may be needed to treat diseases, where editing creates a neutral fitness advantage, relative to diseases where editing creates increased fitness for target cells. If editing imposes a fitness disadvantage, as would be the case for restoring function to a tumor suppressor gene in cancer cells, modified cells would be outcompeted by their diseased counterparts, causing the benefit of treatment to be low relative to editing rates. This may be overcome with supplemental therapies to increase the potency and/or fitness of the edited cells relative to the diseased counterparts.

In addition to cell fitness, the amount of gene product necessary to treat disease can also influence the minimal level of therapeutic genome editing that can treat or prevent a disease or a symptom thereof. In cases where a small change in the gene product levels can result in significant changes in clinical outcome, the minimal level of therapeutic genome editing is less relative to cases where a larger change in the gene product levels are needed to gain a clinically relevant response. In one embodiment, the minimal level of therapeutic genome editing can range from 0.1 to 1%, 1-5%, 5-10%, 10-15%, 15-20%, 20-25%, 25-30%, 30-35%, 35-40%, 40-45%. 45-50%, or 50-55%. Thus, where a small change in gene product levels can influence clinical outcomes and diseases where there is a fitness advantage for edited cells, are ideal targets for genome editing therapy, as the therapeutic modification threshold is low enough to permit a high chance of success.

The activity of NHEJ and HDR DSB repair can vary by cell type and cell state. NHEJ is not highly regulated by the cell cycle and is efficient across cell types, allowing for high levels of gene disruption in accessible target cell populations. In contrast, HDR acts primarily during S/G2 phase, and is therefore restricted to cells that are actively dividing, limiting treatments that require precise genome modifications to mitotic cells [Ciccia, A. & Elledge, S. J. Molecular cell 40, 179-204 (2010); Chapman, J. R., et al. Molecular cell 47, 497-510 (2012)].

The efficiency of correction via HDR may be controlled by the epigenetic state or sequence of the targeted locus, or the specific repair template configuration (single vs. double stranded, long vs. short homology arms) used [Hacein-Bey-Abina, S., et al. The New England journal of medicine 346, 1185-1193 (2002); Gaspar, H. B., et al. Lancet 364, 2181-2187 (2004); Beumer, K. J., et al. G3 (2013)]. The relative activity of NHEJ and HDR machineries in target cells may also affect gene correction efficiency, as these pathways may compete to resolve DSBs [Beumer, K. J., et al. Proceedings of the National Academy of Sciences of the United States of America 105, 19821-19826 (2008)]. HDR also imposes a delivery challenge not seen with NHEJ strategies, as it uses the concurrent delivery of nucleases and repair templates. Thus, these differences can be kept in mind when designing, optimizing, and/or selecting a IscB polypeptide nuclease based therapeutic as described in greater detail elsewhere herein.

IsrB polypeptide nuclease-based polynucleotide modification application can include combinations of proteins, small RNA molecules, and/or repair templates, and can make, In one embodiment, delivery of these multiple parts substantially more challenging than, for example, traditional small molecule therapeutics. Two main strategies for delivery of compositions, systems, and components thereof have been developed: ex vivo and in vivo. In one embodiment of ex vivo treatments, diseased cells are removed from a subject, edited and then transplanted back into the patient. In other embodiments, cells from a healthy allogeneic donor are collected, modified using a composition or component thereof, to impart various functionalities and/or reduce immunogenicity, and administered to an allogeneic recipient in need of treatment. Ex vivo editing has the advantage of allowing the target cell population to be well defined and the specific dosage of therapeutic molecules delivered to cells to be specified. The latter consideration may be particularly important when off-target modifications are a concern, as titrating the amount of nuclease may decrease such mutations (Hsu et al., 2013). Another advantage of ex vivo approaches is the typically high editing rates that can be achieved, due to the development of efficient delivery systems for proteins and nucleic acids into cells in culture for research and gene therapy applications.

In vivo polynucleotide modification via compositions, systems, and/or components thereof involves direct delivery of the compositions, systems, and/or components thereof to cell types in their native tissues. In vivo polynucleotide modification via compositions, systems, and/or components thereof allows diseases in which the affected cell population is not amenable to ex vivo manipulation to be treated. Furthermore, delivering compositions, systems, and/or components thereof to cells in situ allows for the treatment of multiple tissue and cell types.

In one embodiment, such as those where viral vector systems are used to generate viral particles to deliver the composition and/or component thereof to a cell, the total cargo size of the composition and/or component thereof should be considered as vector systems can have limits on the size of a polynucleotide that can be expressed therefrom and/or packaged into cargo inside of a viral particle. In one embodiment, the tropism of a vector system, such as a viral vector system, should be considered as it can impact the cell type to which the composition or component thereof can be efficiently and/or effectively delivered.

When delivering a system or component thereof via a viral-based system, it can be important to consider the amount of viral particles that will be needed to achieve a therapeutic effect so as to account for the potential immune response that can be elicited by the viral particles when delivered to a subject or cell(s). When delivering a system or component thereof via a viral based system, it can be important to consider mechanisms of controlling the distribution and/or dosage of the system in vivo. Generally, to reduce the potential for off-target effects, it is optimal but not necessarily required, that the amount of the system be as close to the minimum or least effective dose. In practice this can be challenging to do.

In one embodiment, it can be important to consider the immunogenicity of the system or component thereof. In embodiments, where the immunogenicity of the system or component thereof is of concern, the immunogenicity system or component thereof can be reduced. By way of example only, the immunogenicity of the system or component thereof can be reduced using the approach set out in Tangri et al. Accordingly, directed evolution or rational design may be used to reduce the immunogenicity of the IsrB polypeptide nuclease in the host species (human or other species).

### Xenotransplantation

The present invention also contemplates use of the composition described herein, e.g. IsrB polypeptide nuclease protein systems, to provide RNA-guided DNA nucleases adapted to be used to provide modified tissues for transplantation. For example, RNA-guided DNA nucleases may be used to knockout, knockdown or disrupt selected genes in an animal, such as a transgenic pig (such as the human heme oxygenase-1 transgenic pig line), for example by disrupting expression of genes that encode epitopes recognized by the human immune system, i.e. xenoantigen genes. Candidate porcine genes for disruption may for example include α(1,3)-galactosyltransferase and cytidine monophosphate-N-acetylneuraminic acid hydroxylase genes (see PCT Patent Publication WO 2014/066505). In addition, genes encoding endogenous retroviruses may be disrupted, for example the genes encoding all porcine endogenous retroviruses (see Yang et al., 2015, Genome-wide inactivation of porcine endogenous retroviruses (PERVs), Science 27 Nov. 2015: Vol. 350 no. 6264 pp. 1101-1104). In addition, RNA-guided DNA nucleases may be used to target a site for integration of additional genes in xenotransplant donor animals, such as a human CD55 gene to improve protection against hyperacute rejection.

Embodiments of the invention also relate to methods and compositions related to knocking out genes, amplifying genes and repairing particular mutations associated with DNA repeat instability and neurological disorders (Robert D. Wells, Tetsuo Ashizawa, Genetic Instabilities and Neurological Diseases, Second Edition, Academic Press, Oct. 13, 2011-Medical). Specific aspects of tandem repeat sequences have been found to be responsible for more than twenty human diseases (New insights into repeat instability: role of RNA.DNA hybrids. McIvor E I, Polak U, Napierala M. RNA Biol. 2010 September-Oct; 7 (5): 551-8). The present effector protein systems may be harnessed to correct these defects of genomic instability.

Several further aspects of the invention relate to correcting defects associated with a wide range of genetic diseases which are further described on the website of the National Institutes of Health under the topic subsection Genetic Disorders (website at health.nih.gov/topic/GeneticDisorders). The genetic brain diseases may include but are not limited to Adrenoleukodystrophy, Agenesis of the Corpus Callosum, Aicardi Syndrome, Alpers' Disease, Alzheimer's Disease, Barth Syndrome, Batten Disease, CADASIL, Cerebellar Degeneration, Fabry's Disease, Gerstmann-Straussler-Scheinker Disease, Huntington's Disease and other Triplet Repeat Disorders, Leigh's Disease, Lesch-Nyhan Syndrome, Menkes Disease, Mitochondrial Myopathies and NINDS Colpocephaly. These diseases are further described on the website of the National Institutes of Health under the subsection Genetic Brain Disorders.

### Applications in Plants and Fungi

The compositions, systems, and methods described herein can be used to perform gene or genome interrogation or editing or manipulation in plants and fungi. For example, the applications include investigation and/or selection and/or interrogations and/or comparison and/or manipulations and/or transformation of plant genes or genomes; e.g., to create, identify, develop, optimize, or confer trait(s) or characteristic(s) to plant(s) or to transform a plant or fugus genome. There can accordingly be improved production of plants, new plants with new combinations of traits or characteristics or new plants with enhanced traits. The compositions, systems, and methods can be used with regard to plants in Site-Directed Integration (SDI) or Gene Editing (GE) or any Near Reverse Breeding (NRB) or Reverse Breeding (RB) techniques.

The compositions, systems, and methods herein may be used to confer desired traits (e.g., enhanced nutritional quality, increased resistance to diseases and resistance to biotic and abiotic stress, and increased production of commercially valuable plant products or heterologous compounds) on essentially any plants and fungi, and their cells and tissues. The compositions, systems, and methods may be used to modify endogenous genes or to modify their expression without the permanent introduction into the genome of any foreign gene.

In one embodiment, compositions, systems, and methods may be used in genome editing in plants or where RNAi or similar genome editing techniques have been used previously; see, e.g., Nekrasov, “Plant genome editing made easy: targeted mutagenesis in model and crop plants using the CRISPR-Cas system,” Plant Methods 2013, 9:39 (doi: 10.1186/1746-4811-9-39); Brooks, “Efficient gene editing in tomato in the first generation using the CRISPR-Cas9 system,” Plant Physiology September 2014 pp 114247577; Shan, “Targeted genome modification of crop plants using a CRISPR-Cas system,” Nature Biotechnology 31, 686-688 (2013); Feng, “Efficient genome editing in plants using a CRISPR/Cas system,” Cell Research (2013) 23:1229-1232. doi: 10.1038/cr.2013.114; published online 20 Aug. 2013; Xie, “RNA-guided genome editing in plants using a CRISPR-Cas system,” Mol Plant. 2013 November; 6 (6): 1975-83. doi: 10.1093/mp/sst119. Epub 2013 Aug. 17; Xu, “Gene targeting using the Agrobacterium tumefaciens-mediated CRISPR-Cas system in rice,” Rice 2014, 7:5 (2014), Zhou et al., “Exploiting SNPs for biallelic CRISPR mutations in the outcrossing woody perennial Populus reveals 4-coumarate: CoA ligase specificity and Redundancy,” New Phytologist (2015) (Forum) 1-4 (available online only at www.newphytologist.com); Caliando et al, “Targeted DNA degradation using a CRISPR device stably carried in the host genome, NATURE COMMUNICATIONS 6:6989, DOI: 10.1038/ncomms7989, www.nature.com/naturecommunications DOI: 10.1038/ncomms7989; U.S. Pat. No. 6,603,061-Agrobacterium-Mediated Plant Transformation Method; U.S. Pat. No. 7,868,149-Plant Genome Sequences and Uses Thereof and US 2009/0100536-Transgenic Plants with Enhanced Agronomic Traits, Morrell et al “Crop genomics: advances and applications,” Nat Rev Genet. 2011 Dec. 29; 13 (2): 85-96, all the contents and disclosure of each of which are herein incorporated by reference in their entirety. Aspects of utilizing the compositions, systems, and methods may be analogous to the use of the composition in plants, and mention is made of the University of Arizona website “CRISPR-PLANT” (genome.arizona.edu/crispr/) (supported by Penn State and AGI).

The compositions, systems, and methods may also be used on protoplasts. A “protoplast” refers to a plant cell that has had its protective cell wall completely or partially removed using, for example, mechanical or enzymatic means resulting in an intact biochemical competent unit of living plant that can reform their cell wall, proliferate and regenerate grow into a whole plant under proper growing conditions.

The compositions, systems, and methods may be used for screening genes (e.g., endogenous, mutations) of interest. In some examples, genes of interest include those encoding enzymes involved in the production of a component of added nutritional value or generally genes affecting agronomic traits of interest, across species, phyla, and plant kingdom. By selectively targeting e.g. genes encoding enzymes of metabolic pathways, the genes responsible for certain nutritional aspects of a plant can be identified. Similarly, by selectively targeting genes which may affect a desirable agronomic trait, the relevant genes can be identified. Accordingly, the present invention encompasses screening methods for genes encoding enzymes involved in the production of compounds with a particular nutritional value and/or agronomic traits.

It is also understood that reference herein to animal cells may also apply, mutatis mutandis, to plant or fungal cells unless otherwise apparent; and the enzymes herein having reduced off-target effects and systems employing such enzymes can be used in plant applications, including those mentioned herein.

In some cases, nucleic acids introduced to plants and fungi may be codon optimized for expression in the plants and fungi. Methods of codon optimization include those described in Kwon K C, et al., Codon Optimization to Enhance Expression Yields Insights into Chloroplast Translation, Plant Physiol. 2016 September; 172 (1): 62-77.

The components (e.g., IsrB polypeptide nuclease) in the compositions and systems may further comprise one or more functional domains described herein. In some examples, the functional domains may be an exonuclease. Such exonuclease may increase the efficiency of the IsrB polypeptide nuclease′ function, e.g., mutagenesis efficiency. An example of the functional domain is Trex2, in Weiss T et al., as described www.biorxiv.org/content/10.1101/2020.04.11.037572v1, doi:
doi.org/10.1101/2020.04.11.037572.

### Examples of Plants

The compositions, systems, and methods herein can be used to confer desired traits on essentially any plant. A wide variety of plants and plant cell systems may be engineered for the desired physiological and agronomic characteristics. In general, the term “plant” relates to any various photosynthetic, eukaryotic, unicellular or multicellular organism of the kingdom Plantae characteristically growing by cell division, containing chloroplasts, and having cell walls comprised of cellulose. The term plant encompasses monocotyledonous and dicotyledonous plants.

The compositions, systems, and methods may be used over a broad range of plants, such as for example with dicotyledonous plants belonging to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales; monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g., those belonging to the orders Pinales, Ginkgoales, Cycadales, Araucariales, Cupressales and Gnetales.

The compositions, systems, and methods herein can be used over a broad range of plant species, included in the non-limitative list of dicot, monocot or gymnosperm genera hereunder: Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, and Pseudotsuga.

In one embodiment, target plants and plant cells for engineering include those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Specifically, the plants are intended to comprise without limitation angiosperm and gymnosperm plants such as acacia, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini.

The term plant also encompasses Algae, which are mainly photoautotrophs unified primarily by their lack of roots, leaves and other organs that characterize higher plants. The compositions, systems, and methods can be used over a broad range of “algae” or “algae cells.” Examples of algae include eukaryotic phyla, including the Rhodophyta (red algae), Chlorophyta (green algae), Phaeophyta (brown algae), Bacillariophyta (diatoms), Eustigmatophyta and dinoflagellates as well as the prokaryotic phylum Cyanobacteria (blue-green algae). Examples of algae species include those of Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium.

### Plant Promoters

In order to ensure appropriate expression in a plant cell, the components of the components and systems herein may be placed under control of a plant promoter. A plant promoter is a promoter operable in plant cells. A plant promoter is capable of initiating transcription in plant cells, whether or not its origin is a plant cell. The use of different types of promoters is envisaged.

In some examples, the plant promoter is a constitutive plant promoter, which is a promoter that is able to express the open reading frame (ORF) that it controls in all or nearly all of the plant tissues during all or nearly all developmental stages of the plant (referred to as “constitutive expression”). One example of a constitutive promoter is the cauliflower mosaic virus 35S promoter. In some examples, the plant promoter is a regulated promoter, which directs gene expression not constitutively, but in a temporally- and/or spatially-regulated manner, and includes tissue-specific, tissue-preferred and inducible promoters. Different promoters may direct the expression of a gene in different tissues or cell types, or at different stages of development, or in response to different environmental conditions. In some examples, the plant promoter is a tissue-preferred promoters, which can be utilized to target enhanced expression in certain cell types within a particular plant tissue, for instance vascular cells in leaves or roots or in specific cells of the seed.

Exemplary plant promoters include those obtained from plants, plant viruses, and bacteria such as Agrobacterium or Rhizobium which comprise genes expressed in plant cells. Additional examples of promoters include those described in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

In some examples, a plant promoter may be an inducible promoter, which is inducible and allows for spatiotemporal control of gene editing or gene expression may use a form of energy. The form of energy may include sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. In a particular example, of the components of a light inducible system include a IscB polypeptide nuclease, a light-responsive cytochrome heterodimer (e.g. from Arabidopsis thaliana), and a transcriptional activation/repression domain.

In some examples, the promoter may be a chemical-regulated promotor (where the application of an exogenous chemical induces gene expression) or a chemical-repressible promoter (where application of the chemical represses gene expression). Examples of chemical-inducible promoters include maize In2-2 promoter (activated by benzene sulfonamide herbicide safeners), the maize GST promoter (activated by hydrophobic electrophilic compounds used as pre-emergent herbicides), the tobacco PR-1 a promoter (activated by salicylic acid), promoters regulated by antibiotics (such as tetracycline-inducible and tetracycline-repressible promoters).

### Stable Integration in the Genome of Plants

In one embodiment, polynucleotides encoding the components of the compositions and systems may be introduced for stable integration into the genome of a plant cell. In some cases, vectors or expression systems may be used for such integration. The design of the vector or the expression system can be adjusted depending on for when, where and under what conditions the guide RNA and/or the IsrB polypeptide nuclease gene are expressed. In some cases, the polynucleotides may be integrated into an organelle of a plant, such as a plastid, mitochondrion or a chloroplast. The elements of the expression system may be on one or more expression constructs which are either circular such as a plasmid or transformation vector, or non-circular such as linear double stranded DNA.

In one embodiment, the method of integration generally comprises the steps of selecting a suitable host cell or host tissue, introducing the construct(s) into the host cell or host tissue, and regenerating plant cells or plants therefrom. In some examples, the expression system for stable integration into the genome of a plant cell may contain one or more of the following elements: a promoter element that can be used to express the RNA and/or IsrB polypeptide nuclease in a plant cell; a 5′ untranslated region to enhance expression; an intron element to further enhance expression in certain cells, such as monocot cells; a multiple-cloning site to provide convenient restriction sites for inserting the guide RNA and/or the IsrB polypeptide nuclease gene sequences and other desired elements; and a 3′ untranslated region to provide for efficient termination of the expressed transcript.

### Transient Expression in Plants

In one embodiment, the components of the compositions and systems may be transiently expressed in the plant cell. In some examples, the compositions and systems may modify a target nucleic acid only when both the ωRNA and the IsrB polypeptide nuclease are present in a cell, such that genomic modification can further be controlled. As the expression of the IsrB polypeptide nuclease is transient, plants regenerated from such plant cells typically contain no foreign DNA. In certain examples, the IsrB polypeptide is stably expressed and the guide sequence is transiently expressed.

DNA and/or RNA (e.g., mRNA) may be introduced to plant cells for transient expression. In such cases, the introduced nucleic acid may be provided in sufficient quantity to modify the cell but do not persist after a contemplated period of time has passed or after one or more cell divisions.

The transient expression may be achieved using suitable vectors. Exemplary vectors that may be used for transient expression include a PEAQ vector (may be tailored for Agrobacterium-mediated transient expression) and Cabbage Leaf Curl virus (CaLCuV), and vectors described in Sainsbury F. et al., Plant Biotechnol J. 2009 September; 7 (7): 682-93; and Yin K et al., Scientific Reports volume 5, Article number: 14926 (2015).

Combinations of the different methods described above are also envisaged.

Translocation to and/or Expression in Specific Plant Organelles

The compositions and systems herein may comprise elements for translocation to and/or expression in a specific plant organelle.

### Chloroplast Targeting

In one embodiment, it is envisaged that the compositions and systems are used to specifically modify chloroplast genes or to ensure expression in the chloroplast. The compositions and systems (e.g., IsrB polypeptide nuclease, ωRNAs, or their encoding polynucleotides) may be transformed, compartmentalized, and/or targeted to the chloroplast. In an example, the introduction of genetic modifications in the plastid genome can reduce biosafety issues such as gene flow through pollen.

Examples of methods of chloroplast transformation include Particle bombardment, PEG treatment, and microinjection, and the translocation of transformation cassettes from the nuclear genome to the plastid. In some examples, targeting of chloroplasts may be achieved by incorporating in chloroplast localization sequence, and/or the expression construct a sequence encoding a chloroplast transit peptide (CTP) or plastid transit peptide, operably linked to the 5′ region of the sequence encoding the components of the compositions and systems. Additional examples of transforming, targeting and localization of chloroplasts include those described in WO2010061186, Protein Transport into Chloroplasts, 2010, Annual Review of Plant Biology, Vol. 61:157-180, and US20040142476, which are incorporated by reference herein in their entireties.

### Exemplary Applications in Plants

The compositions, systems, and methods may be used to generate genetic variation(s) in a plant (e.g., crop) of interest. One or more, e.g., a library of, ωRNAs targeting one or more locations in a genome may be provided and introduced into plant cells together with the IsrB polypeptide nuclease. For example, a collection of genome-scale point mutations and gene knock-outs can be generated. In some examples, the compositions, systems, and methods may be used to generate a plant part or plant from the cells so obtained and screening the cells for a trait of interest. The target genes may include both coding and non-coding regions. In some cases, the trait is stress tolerant and the method is a method for the generation of stress-tolerant crop varieties.

In one embodiment, the compositions, systems, and methods are used to modify endogenous genes or to modify their expression. The expression of the components may induce targeted modification of the genome, either by direct activity of the IsrB polypeptide nuclease and optionally introduction of recombination template DNA, or by modification of genes targeted. The different strategies described herein above allow IsrB polypeptide nuclease-mediated targeted genome editing without requiring the introduction of the components into the plant genome.

In some cases, the modification may be performed without the permanent introduction into the genome of the plant of any foreign gene, including those encoding components of the composition herein, so as to avoid the presence of foreign DNA in the genome of the plant. This can be of interest as the regulatory requirements for non-transgenic plants are less rigorous. Components which are transiently introduced into the plant cell are typically removed upon crossing.

For example, the modification may be performed by transient expression of the components of the compositions and systems. The transient expression may be performed by delivering the components of the compositions and systems with viral vectors, delivery into protoplasts, with the aid of particulate molecules such as nanoparticles or CPPs.

Generation of Plants with Desired Traits

The compositions, systems, and methods herein may be used to introduce desired traits to plants. The approaches include introduction of one or more foreign genes to confer a trait of interest, editing or modulating endogenous genes to confer a trait of interest.

### Agronomic Traits

In one embodiment, crop plants can be improved by influencing specific plant traits. Examples of the traits include improved agronomic traits such as herbicide resistance, disease resistance, abiotic stress tolerance, high yield, and superior quality, pesticide-resistance, disease resistance, insect and nematode resistance, resistance against parasitic weeds, drought tolerance, nutritional value, stress tolerance, self-pollination voidance, forage digestibility biomass, and grain yield.

In one embodiment, genes that confer resistance to pests or diseases may be introduced to plants. In cases there are endogenous genes that confer such resistance in plants, their expression and function may be enhanced (e.g., by introducing extra copies, modifications that enhance expression and/or activity).

Examples of genes that confer resistance include plant disease resistance genes (e.g., Cf-9, Pto, RSP2, SIDMR6-1), genes conferring resistance to a pest (e.g., those described in WO96/30517), Bacillus thuringiensis proteins, lectins, Vitamin-binding proteins (e.g., avidin), enzyme inhibitors (e.g., protease or proteinase inhibitors or amylase inhibitors), insect-specific hormones or pheromones (e.g., ecdysteroid or a juvenile hormone, variant thereof, a mimetic based thereon, or an antagonist or agonist thereof) or genes involved in the production and regulation of such hormone and pheromones, insect-specific peptides or neuropeptide, Insect-specific venom (e.g., produced by a snake, a wasp, etc., or analog thereof), Enzymes responsible for a hyperaccumulation of a monoterpene, a sesquiterpene, a steroid, hydroxamic acid, a phenylpropanoid derivative or another nonprotein molecule with insecticidal activity, Enzymes involved in the modification of biologically active molecule (e.g., a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic), molecules that stimulates signal transduction, Viral-invasive proteins or a complex toxin derived therefrom, Developmental-arrestive proteins produced in nature by a pathogen or a parasite, a developmental-arrestive protein produced in nature by a plant, or any combination thereof.

The compositions, systems, and methods may be used to identify, screen, introduce or remove mutations or sequences lead to genetic variability that give rise to susceptibility to certain pathogens, e.g., host specific pathogens. Such approach may generate plants that are non-host resistance, e.g., the host and pathogen are incompatible or there can be partial resistance against all races of a pathogen, typically controlled by many genes and/or also complete resistance to some races of a pathogen but not to other races.

In one embodiment, the compositions, systems, and methods may be used to modify genes involved in plant diseases. Such genes may be removed, inactivated, or otherwise regulated or modified. Examples of plant diseases include those described in [0045]-[0080] of US20140213619A1, which is incorporated by reference herein in its entirety.

In one embodiment, genes that confer resistance to herbicides may be introduced to plants. Examples of genes that confer resistance to herbicides include genes conferring resistance to herbicides that inhibit the growing point or meristem, such as an imidazolinone or a sulfonylurea, genes conferring glyphosate tolerance (e.g., resistance conferred by, e.g., mutant 5-enolpyruvylshikimate-3-phosphate synthase genes, aroA genes and glyphosate acetyl transferase (GAT) genes, respectively), or resistance to other phosphono compounds such as by glufosinate (phosphinothricin acetyl transferase (PAT) genes from Streptomyces species, including Streptomyces hygroscopicus and Streptomyces viridichromogenes), and to pyridinoxy or phenoxy proprionic acids and cyclohexones by ACCase inhibitor-encoding genes), genes conferring resistance to herbicides that inhibit photosynthesis (such as a triazine (psbA and gs+ genes) or a benzonitrile (nitrilase gene), and glutathione S-transferase), genes encoding enzymes detoxifying the herbicide or a mutant glutamine synthase enzyme that is resistant to inhibition, genes encoding a detoxifying enzyme is an enzyme encoding a phosphinothricin acetyltransferase (such as the bar or pat protein from Streptomyces species), genes encoding hydroxyphenylpyruvatedioxygenases (HPPD) inhibitors, e.g., naturally occurring HPPD resistant enzymes, and genes encoding a mutated or chimeric HPPD enzyme.

In one embodiment, genes involved in Abiotic stress tolerance may be introduced to plants. Examples of genes include those capable of reducing the expression and/or the activity of poly (ADP-ribose) polymerase (PARP) gene, transgenes capable of reducing the expression and/or the activity of the PARG encoding genes, genes coding for a plant-functional enzyme of the nicotineamide adenine dinucleotide salvage synthesis pathway including nicotinamidase, nicotinate phosphoribosyltransferase, nicotinic acid mononucleotide adenyl transferase, nicotinamide adenine dinucleotide synthetase or nicotine amide phosphorybosyltransferase, enzymes involved in carbohydrate biosynthesis, enzymes involved in the production of polyfructose (e.g., the inulin and levan-type), the production of alpha-1,6 branched alpha-1,4-glucans, the production of alternan, the production of hyaluronan.

In one embodiment, genes that improve drought resistance may be introduced to plants. Examples of genes Ubiquitin Protein Ligase protein (UPL) protein (UPL3), DR02, DR03, ABC transporter, and DREB1A.

### Nutritionally Improved Plants

In one embodiment, the compositions, systems, and methods may be used to produce nutritionally improved plants. In some examples, such plants may provide functional foods, e.g., a modified food or food ingredient that may provide a health benefit beyond the traditional nutrients it contains. In certain examples, such plants may provide nutraceuticals foods, e.g., substances that may be considered a food or part of a food and provides health benefits, including the prevention and treatment of disease. The nutraceutical foods may be useful in the prevention and/or treatment of diseases in animals and humans, e.g., cancers, diabetes, cardiovascular disease, and hypertension.

An improved plant may naturally produce one or more desired compounds and the modification may enhance the level or activity or quality of the compounds. In some cases, the improved plant may not naturally produce the compound(s), while the modification enables the plant to produce such compound(s). In some cases, the compositions, systems, and methods used to modify the endogenous synthesis of these compounds indirectly, e.g. by modifying one or more transcription factors that controls the metabolism of this compound.

Examples of nutritionally improved plants include plants comprising modified protein quality, content and/or amino acid composition, essential amino acid contents, oils and fatty acids, carbohydrates, vitamins and carotenoids, functional secondary metabolites, and minerals. In some examples, the improved plants may comprise or produce compounds with health benefits. Examples of nutritionally improved plants include those described in Newell-McGloughlin, Plant Physiology, July 2008, Vol. 147, pp. 939-953.

Examples of compounds that can be produced include carotenoids (e.g., α-Carotene or β-Carotene), lutein, lycopene, Zeaxanthin, Dietary fiber (e.g., insoluble fibers, β-Glucan, soluble fibers, fatty acids (e.g., ω-3 fatty acids, Conjugated linoleic acid, GLA,), Flavonoids (e.g., Hydroxycinnamates, flavonols, catechins and tannins), Glucosinolates, indoles, isothiocyanates (e.g., Sulforaphane), Phenolics (e.g., stilbenes, caffeic acid and ferulic acid, epicatechin), Plant stanols/sterols, Fructans, inulins, fructo-oligosaccharides, Saponins, Soybean proteins, Phytoestrogens (e.g., isoflavones, lignans), Sulfides and thiols such as diallyl sulphide, Allyl methyl trisulfide, dithiolthiones, Tannins, such as proanthocyanidins, or any combination thereof.

The compositions, systems, and methods may also be used to modify protein/starch functionality, shelf life, taste/aesthetics, fiber quality, and allergen, antinutrient, and toxin reduction traits.

Examples of genes and nucleic acids that can be modified to introduce the traits include stearyl-ACP desaturase, DNA associated with the single allele which may be responsible for maize mutants characterized by low levels of phytic acid, Tf RAP2.2 and its interacting partner SINAT2, Tf Dof1, and DOF Tf AtDof1.1 (OBP2).

### Modification of Polyploid Plants

The compositions, systems, and methods may be used to modify polyploid plants. Polyploid plants carry duplicate copies of their genomes (e.g. as many as six, such as in wheat). In some cases, the compositions, systems, and methods may be/can be multiplexed to affect all copies of a gene, or to target dozens of genes at once. For instance, the compositions, systems, and methods may be used to simultaneously ensure a loss of function mutation in different genes responsible for suppressing defenses against a disease. The modification may be simultaneous suppression the expression of the TaMLO-A1, TaMLO-B1 and TaMLO-D1 nucleic acid sequence in a wheat plant cell and regenerating a wheat plant therefrom, in order to ensure that the wheat plant is resistant to powdery mildew (e.g., as described in WO2015109752).

### Regulation of Fruit-Ripening

The compositions, systems, and methods may be used to regulate ripening of fruits. Ripening is a normal phase in the maturation process of fruits and vegetables. Only a few days after it starts it may render a fruit or vegetable inedible, which can bring significant losses to both farmers and consumers.

In one embodiment, the compositions, systems, and methods are used to reduce ethylene production. In some examples, the compositions, systems, and methods may be used to suppress the expression and/or activity of ACC synthase, insert a ACC deaminase gene or a functional fragment thereof, insert a SAM hydrolase gene or functional fragment thereof, suppress ACC oxidase gene expression

Alternatively or additionally, the compositions, systems, and methods may be used to modify ethylene receptors (e.g., suppressing ETR1) and/or Polygalacturonase (PG). Suppression of a gene may be achieved by introducing a mutation, an antisense sequence, and/or a truncated copy of the gene to the genome.

### Increasing Storage Life of Plants

In one embodiment, the compositions, systems, and methods are used to modify genes involved in the production of compounds which affect storage life of the plant or plant part. The modification may be in a gene that prevents the accumulation of reducing sugars in potato tubers. Upon high-temperature processing, these reducing sugars react with free amino acids, resulting in brown, bitter-tasting products and elevated levels of acrylamide, which is a potential carcinogen. In one embodiment, the methods provided herein are used to reduce or inhibit expression of the vacuolar invertase gene (VInv), which encodes a protein that breaks down sucrose to glucose and fructose.

### Reducing Allergens in Plants

In one embodiment, the compositions, systems, and methods are used to generate plants with a reduced level of allergens, making them safer for consumers. To this end, the compositions, systems, and methods may be used to identify and modify (e.g., suppress) one or more genes responsible for the production of plant allergens. Examples of such genes include Lol p5, as well as those in peanuts, soybeans, lentils, peas, lupin, green beans, mung beans, such as those described in Nicolaou et al., Current Opinion in Allergy and Clinical Immunology 2011; 11 (3): 222), which is incorporated by reference herein in its entirety.

### Generation of Male Sterile Plants

The compositions, systems, and methods may be used to generate male sterile plants. Hybrid plants typically have advantageous agronomic traits compared to inbred plants. However, for self-pollinating plants, the generation of hybrids can be challenging. In different plant types (e.g., maize and rice), genes have been identified which are important for plant fertility, more particularly male fertility. Plants that are as such genetically altered can be used in hybrid breeding programs.

The compositions, systems, and methods may be used to modify genes involved male fertility, e.g., inactivating (such as by introducing mutations to) genes required for male fertility. Examples of the genes involved in male fertility include cytochrome P450-like gene (MS26) or the meganuclease gene (MS45), and those described in Wan X et al., Mol Plant. 2019 Mar. 4; 12 (3): 321-342; and Kim Y J, et al., Trends Plant Sci. 2018 January; 23 (1): 53-65.

### Increasing the Fertility Stage in Plants

In one embodiment, the compositions, systems, and methods may be used to prolong the fertility stage of a plant such as of a rice. For instance, a rice fertility stage gene such as Ehd3 can be targeted in order to generate a mutation in the gene and plantlets can be selected for a prolonged regeneration plant fertility stage.

### Production of Early Yield of Products

In one embodiment, the compositions, systems, and methods may be used to produce early yield of the product. For example, flowering process may be modulated, e.g., by mutating flowering repressor gene such as SP5G. Examples of such approaches include those described in Soyk S, et al., Nat Genet. 2017 January; 49 (1): 162-168.

### Oil and Biofuel Production

The compositions, systems, and methods may be used to generate plants for oil and biofuel production. Biofuels include fuels made from plant and plant-derived resources. Biofuels may be extracted from organic matter whose energy has been obtained through a process of carbon fixation or are made through the use or conversion of biomass. This biomass can be used directly for biofuels or can be converted to convenient energy containing substances by thermal conversion, chemical conversion, and biochemical conversion. This biomass conversion can result in fuel in solid, liquid, or gas form. Biofuels include bioethanol and biodiesel. Bioethanol can be produced by the sugar fermentation process of cellulose (starch), which may be derived from maize and sugar cane. Biodiesel can be produced from oil crops such as rapeseed, palm, and soybean. Biofuels can be used for transportation.

### Generation of Plants for Production of Vegetable Oils and Biofuels

The compositions, systems, and methods may be used to generate algae (e.g., diatom) and other plants (e.g., grapes) that express or overexpress high levels of oil or biofuels.

In some cases, the compositions, systems, and methods may be used to modify genes involved in the modification of the quantity of lipids and/or the quality of the lipids. Examples of such genes include those involved in the pathways of fatty acid synthesis, e.g., acetyl-CoA carboxylase, fatty acid synthase, 3-ketoacyl_acyl-carrier protein synthase III, glycerol-3-phospate deshydrogenase (G3PDH), Enoyl-acyl carrier protein reductase (Enoyl-ACP-reductase), glycerol-3-phosphate acyltransferase, lysophosphatidic acyl transferase or diacylglycerol acyltransferase, phospholipid: diacylglycerol acyltransferase, phoshatidate phosphatase, fatty acid thioesterase such as palmitoyi protein thioesterase, or malic enzyme activities.

In further embodiments, it is envisaged to generate diatoms that have increased lipid accumulation. This can be achieved by targeting genes that decrease lipid catabolization. Examples of genes include those involved in the activation of triacylglycerol and free fatty acids, β-oxidation of fatty acids, such as genes of acyl-CoA synthetase, 3-ketoacyl-CoA thiolase, acyl-CoA oxidase activity and phosphoglucomutase.

In some examples, algae may be modified for production of oil and biofuels, including fatty acids (e.g., fatty esters such as acid methyl esters (FAME) and fatty acid ethyl esters (FAEE)). Examples of methods of modifying microalgae include those described in Stovicek et al. Metab. Eng. Comm., 2015; 2:1; U.S. Pat. No. 8,945,839; and International Patent Publication No. WO 2015/086795.

In some examples, one or more genes may be introduced (e.g., overexpressed) to the plants (e.g., algae) to produce oils and biofuels (e.g., fatty acids) from a carbon source (e.g., alcohol). Examples of the genes include genes encoding acyl-CoA synthases, ester synthases, thioesterases (e.g., tesA, ‘tesA, tesB, fatB, fatB2, fatB3, fatA1, or fatA), acyl-CoA synthases (e.g., fadD, JadK, BH3103, pfl-4354, EAV15023, fadD1, fadD2, RPC_4074, fadDD35, fadDD22, faa39), ester synthases (e.g., synthase/acyl-CoA: diacylglycerl acyltransferase from Simmondsia chinensis, Acinetobacter sp. ADP, Alcanivorax borkumensis, Pseudomonas aeruginosa, Fundibacter jadensis, Arabidopsis thaliana, or Alkaligenes eutrophus, or variants thereof).

Additionally or alternatively, one or more genes in the plants (e.g., algae) may be inactivated (e.g., expression of the genes is decreased). For examples, one or more mutations may be introduced to the genes. Examples of such genes include genes encoding acyl-CoA dehydrogenases (e.g., fade), outer membrane protein receptors, and transcriptional regulator (e.g., repressor) of fatty acid biosynthesis (e.g., fabR), pyruvate formate lyases (e.g., pflB), lactate dehydrogenases (e.g., IdhA).

### Organic Acid Production

In one embodiment, plants may be modified to produce organic acids such as lactic acid. The plants may produce organic acids using sugars, pentose or hexose sugars. To this end, one or more genes may be introduced (e.g., and overexpressed) in the plants. An example of such genes include LDH gene.

In some examples, one or more genes may be inactivated (e.g., expression of the genes is decreased). For examples, one or more mutations may be introduced to the genes. The genes may include those encoding proteins involved an endogenous metabolic pathway which produces a metabolite other than the organic acid of interest and/or wherein the endogenous metabolic pathway consumes the organic acid.

Examples of genes that can be modified or introduced include those encoding pyruvate decarboxylases (pdc), fumarate reductases, alcohol dehydrogenases (adh), acetaldehyde dehydrogenases, phosphoenolpyruvate carboxylases (ppc), D-lactate dehydrogenases (d-ldh), L-lactate dehydrogenases (1-1dh), lactate 2-monooxygenases, lactate dehydrogenase, cytochrome-dependent lactate dehydrogenases (e.g., cytochrome B2-dependent L-lactate dehydrogenases).

### Enhancing Plant Properties for Biofuel Production

In one embodiment, the compositions, systems, and methods are used to alter the properties of the cell wall of plants to facilitate access by key hydrolyzing agents for a more efficient release of sugars for fermentation. By reducing the proportion of lignin in a plant the proportion of cellulose can be increased. In one embodiment, lignin biosynthesis may be downregulated in the plant so as to increase fermentable carbohydrates.

In some examples, one or more lignin biosynthesis genes may be down regulated. Examples of such genes include 4-coumarate 3-hydroxylases (C3H), phenylalanine ammonia-lyases (PAL), cinnamate 4-hydroxylases (C4H), hydroxycinnamoyl transferases (HCT), caffeic acid O-methyltransferases (COMT), caffeoyl CoA 3-O-methyltransferases (CCoAOMT), ferulate 5-hydroxylases (F5H), cinnamyl alcohol dehydrogenases (CAD), cinnamoyl CoA-reductases (CCR), 4-coumarate-CoA ligases (4CL), monolignol-lignin-specific glycosyltransferases, and aldehyde dehydrogenases (ALDH), and those described in WO 2008064289.

In some examples, plant mass that produces lower level of acetic acid during fermentation may be reduced. To this end, genes involved in polysaccharide acetylation (e.g., Cas1L and those described in WO 2010096488) may be inactivated.

### Other Microorganisms for Oils and Biofuel Production

In one embodiment, microorganisms other than plants may be used for production of oils and biofuels using the compositions, systems, and methods herein. Examples of the microorganisms include those of the genus of Escherichia, Bacillus, Lactobacillus, Rhodococcus, Synechococcus, Synechoystis, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces.

### Plant Cultures and Regeneration

In one embodiment, the modified plants or plant cells may be cultured to regenerate a whole plant which possesses the transformed or modified genotype and thus the desired phenotype. Examples of regeneration techniques include those relying on manipulation of certain phytohormones in a tissue culture growth medium, relying on a biocide and/or herbicide marker which has been introduced together with the desired nucleotide sequences, obtaining from cultured protoplasts, plant callus, explants, organs, pollens, embryos or parts thereof.

### Detecting Modifications in the Plant Genome-Selectable Markers

When the compositions, systems, and methods are used to modify a plant, suitable methods may be used to confirm and detect the modification made in the plant. In some examples, when a variety of modifications are made, one or more desired modifications or traits resulting from the modifications may be selected and detected. The detection and confirmation may be performed by biochemical and molecular biology techniques such as Southern analysis, PCR, Northern blot, S1 RNase protection, primer-extension or reverse transcriptase-PCR, enzymatic assays, ribozyme activity, gel electrophoresis, Western blot, immunoprecipitation, enzyme-linked immunoassays, in situ hybridization, enzyme staining, and immunostaining.

In some cases, one or more markers, such as selectable and detectable markers, may be introduced to the plants. Such markers may be used for selecting, monitoring, isolating cells and plants with desired modifications and traits. A selectable marker can confer positive or negative selection and is conditional or non-conditional on the presence of external substrates. Examples of such markers include genes and proteins that confer resistance to antibiotics, such as hygromycin (hpt) and kanamycin (nptII), and genes that confer resistance to herbicides, such as phosphinothricin (bar) and chlorosulfuron (als), enzyme capable of producing or processing a colored substances (e.g., the β-glucuronidase, luciferase, B or C1 genes).

### Applications in Fungi

The compositions, systems, and methods described herein can be used to perform efficient and cost-effective gene or genome interrogation or editing or manipulation in fungi or fungal cells, such as yeast. The approaches and applications in plants may be applied to fungi as well.

A fungal cell may be any type of eukaryotic cell within the kingdom of fungi, such as phyla of Ascomycota, Basidiomycota, Blastocladiomycota, Chytridiomycota, Glomeromycota, Microsporidia, and Neocallimastigomycota. Examples of fungi or fungal cells in include yeasts, molds, and filamentous fungi.

In one embodiment, the fungal cell is a yeast cell. A yeast cell refers to any fungal cell within the phyla Ascomycota and Basidiomycota. Examples of yeasts include budding yeast, fission yeast, and mold, S. cerervisiae, Kluyveromyces marxianus, Issatchenkia orientalis, Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, Pichia kudriavzevii and Candida acidothermophilum).

In one embodiment, the fungal cell is a filamentous fungal cell, which grow in filaments, e.g., hyphae or mycelia. Examples of filamentous fungal cells include Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

In one embodiment, the fungal cell is of an industrial strain. Industrial strains include any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes include fermentation (e.g., in production of food or beverage products), distillation, biofuel production, production of a compound, and production of a polypeptide. Examples of industrial strains include, without limitation, JAY270 and ATCC4124.

In one embodiment, the fungal cell is a polyploid cell whose genome is present in more than one copy. Polyploid cells include cells naturally found in a polyploid state, and cells that has been induced to exist in a polyploid state (e.g., through specific regulation, alteration, inactivation, activation, or modification of meiosis, cytokinesis, or DNA replication). A polyploid cell may be a cell whose entire genome is polyploid, or a cell that is polyploid in a particular genomic locus of interest. In some examples, the abundance of guide RNA may more often be a rate-limiting component in genome engineering of polyploid cells than in haploid cells, and thus the methods using the composition described herein may take advantage of using certain fungal cell types.

In one embodiment, the fungal cell is a diploid cell, whose genome is present in two copies. Diploid cells include cells naturally found in a diploid state, and cells that have been induced to exist in a diploid state (e.g., through specific regulation, alteration, inactivation, activation, or modification of meiosis, cytokinesis, or DNA replication). A diploid cell may refer to a cell whose entire genome is diploid, or it may refer to a cell that is diploid in a particular genomic locus of interest.

In one embodiment, the fungal cell is a haploid cell, whose genome is present in one copy. Haploid cells include cells naturally found in a haploid state, or cells that have been induced to exist in a haploid state (e.g., through specific regulation, alteration, inactivation, activation, or modification of meiosis, cytokinesis, or DNA replication). A haploid cell may refer to a cell whose entire genome is haploid, or it may refer to a cell that is haploid in a particular genomic locus of interest.

The compositions and systems, and nucleic acid encoding thereof may be introduced to fungi cells using the delivery systems and methods herein. Examples of delivery systems include lithium acetate treatment, bombardment, electroporation, and those described in Kawai et al., 2010, Bioeng Bugs. 2010 November-Dec; 1 (6): 395-403.

In some examples, a yeast expression vector (e.g., those with one or more regulatory elements) may be used. Examples of such vectors include a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

### Biofuel and Materials Production by Fungi

In one embodiment, the compositions, systems, and methods may be used for generating modified fungi for biofuel and material productions. For instance, the modified fungi for production of biofuel or biopolymers from fermentable sugars and optionally to be able to degrade plant-derived lignocellulose derived from agricultural waste as a source of fermentable sugars. Foreign genes required for biofuel production and synthesis may be introduced in to fungi In some examples, the genes may encode enzymes involved in the conversion of pyruvate to ethanol or another product of interest, degrade cellulose (e.g., cellulase), endogenous metabolic pathways which compete with the biofuel production pathway.

In some examples, the compositions, systems, and methods may be used for generating and/or selecting yeast strains with improved xylose or cellobiose utilization, isoprenoid biosynthesis, and/or lactic acid production. One or more genes involved in the metabolism and synthesis of these compounds may be modified and/or introduced to yeast cells. Examples of the methods and genes include lactate dehydrogenase, PDC1 and PDC5, and those described in Ha, S. J., et al. (2011) Proc. Natl. Acad. Sci. USA 108 (2): 504-9 and Galazka, J. M., et al. (2010) Science 330 (6000): 84-6; Jakočiūnas T et al., Metab Eng. 2015 March; 28:213-222; Stovicek V, et al., FEMS Yeast Res. 2017 Aug. 1; 17 (5).

### Improved Plants and Yeast Cells

The present disclosure further provides improved plants and fungi. The improved and fungi may comprise one or more genes introduced, and/or one or more genes modified by the compositions, systems, and methods herein. The improved plants and fungi may have increased food or feed production (e.g., higher protein, carbohydrate, nutrient or vitamin levels), oil and biofuel production (e.g., methanol, ethanol), tolerance to pests, herbicides, drought, low or high temperatures, excessive water, etc.

The plants or fungi may have one or more parts that are improved, e.g., leaves, stems, roots, tubers, seeds, endosperm, ovule, and pollen. The parts may be viable, nonviable, regeneratable, and/or non-regeneratable.

The improved plants and fungi may include gametes, seeds, embryos, either zygotic or somatic, progeny and/or hybrids of improved plants and fungi. The progeny may be a clone of the produced plant or fungi, or may result from sexual reproduction by crossing with other individuals of the same species to introgress further desirable traits into their offspring. The cell may be in vivo or ex vivo in the cases of multicellular organisms, particularly plants.

### Further Applications in Plants

Further applications of the compositions, systems, and methods on plants and fungi include visualization of genetic element dynamics (e.g., as described in Chen B, et al., Cell. 2013 Dec. 19; 155 (7): 1479-91), targeted gene disruption positive-selection in vitro and in vivo (as described in Malina A et al., Genes Dev. 2013 Dec. 1; 27 (23): 2602-14), epigenetic modification such as using fusion of IscB polypeptide nuclease and histone-modifying enzymes (e.g., as described in Rusk N, Nat Methods. 2014 January; 11 (1): 28), identifying transcription regulators (e.g., as described in Waldrip Z J, Epigenetics. 2014 September; 9 (9): 1207-11), anti-virus treatment for both RNA and DNA viruses (e.g., as described in Price A A, et al., Proc Natl Acad Sci USA. 2015 May 12; 112 (19): 6164-9; Ramanan V et al., Sci Rep. 2015 Jun. 2; 5:10833), alteration of genome complexity such as chromosome numbers (e.g., as described in Karimi-Ashtiyani R et al., Proc Natl Acad Sci USA. 2015 Sep. 8; 112 (36): 11211-6; Anton T, et al., Nucleus. 2014 March-Apr; 5 (2): 163-72), self-cleavage of the composition for controlled inactivation/activation (e.g., as described Sugano S S et al., Plant Cell Physiol. 2014 March; 55 (3): 475-81), multiplexed gene editing (as described in Kabadi A M et al., Nucleic Acids Res. 2014 Oct. 29; 42 (19): e147), development of kits for multiplex genome editing (as described in Xing H L et al., BMC Plant Biol. 2014 Nov. 29; 14:327), starch production (as described in Hebelstrup K H et al., Front Plant Sci. 2015 Apr. 23; 6:247), targeting multiple genes in a family or pathway (e.g., as described in Ma X et al., Mol Plant. 2015 August; 8 (8): 1274-84), regulation of non-coding genes and sequences (e.g., as described in Lowder L G, et al., Plant Physiol. 2015 October; 169 (2): 971-85), editing genes in trees (e.g., as described in Belhaj K et al., Plant Methods. 2013 Oct. 11; 9 (1): 39; Harrison M M, et al., Genes Dev. 2014 Sep. 1; 28 (17): 1859-72; Zhou X et al., New Phytol. 2015 October; 208 (2): 298-301), introduction of mutations for resistance to host-specific pathogens and pests.

Additional examples of modifications of plants and fungi that may be performed using the compositions, systems, and methods include analogous modifications described in International Patent Publication Nos. WO2016/099887, WO2016/025131, WO2016/073433, WO2017/066175, WO2017/100158, WO 2017/105991, WO2017/106414, WO2016/100272, WO2016/100571, WO 2016/100568, WO 2016/100562, and WO 2017/019867.

### Applications in Non-Human Animals

The compositions, systems, and methods may be used to study and modify non-human animals, e.g., introducing desirable traits and disease resilience, treating diseases, facilitating breeding, etc. In one embodiment, the compositions, systems, and methods may be used to improve breeding and introducing desired traits, e.g., increasing the frequency of trait-associated alleles, introgression of alleles from other breeds/species without linkage drag, and creation of de novo favorable alleles. Genes and other genetic elements that can be targeted may be screened and identified. Examples of application and approaches include those described in Tait-Burkard C, et al., Livestock 2.0-genome editing for fitter, healthier, and more productive farmed animals. Genome Biol. 2018 Nov. 26; 19 (1): 204; Lillico S, Agricultural applications of genome editing in farmed animals. Transgenic Res. 2019 August; 28 (Suppl 2): 57-60; Houston R D, et al., Harnessing genomics to fast-track genetic improvement in aquaculture. Nat Rev Genet. 2020 Apr. 16. doi: 10.1038/s41576-020-0227-y, which are incorporated herein by reference in their entireties. Applications described in other sections such as therapeutic, diagnostic, etc. can also be used on the animals herein.

The compositions, systems, and methods may be used on animals such as fish, amphibians, reptiles, mammals, and birds. The animals may be farm and agriculture animals, or pets. Examples of farm and agriculture animals include horses, goats, sheep, swine, cattle, llamas, alpacas, and birds, e.g., chickens, turkeys, ducks, and geese. The animals may be a non-human primate, e.g., baboons, capuchin monkeys, chimpanzees, lemurs, macaques, marmosets, tamarins, spider monkeys, squirrel monkeys, and vervet monkeys. Examples of pets include dogs, cats horses, wolfs, rabbits, ferrets, gerbils, hamsters, chinchillas, fancy rats, guinea pigs, canaries, parakeets, and parrots.

In one embodiment, one or more genes may be introduced (e.g., overexpressed) in the animals to obtain or enhance one or more desired traits. Growth hormones, insulin-like growth factors (IGF-1) may be introduced to increase the growth of the animals, e.g., pigs or salmon (such as described in Pursel V G et al., J Reprod Fertil Suppl. 1990; 40:235-45; Waltz E, Nature. 2017; 548:148). Fat-1 gene (e.g., from C elegans) may be introduced for production of larger ratio of n-3 to n-6 fatty acids may be induced, e.g. in pigs (such as described in Li M, et al., Genetics. 2018; 8:1747-54). Phytase (e.g., from E. coli) xylanase (e.g., from Aspergillus niger), beta-glucanase (e.g., from bacillus lichenformis) may be introduced to reduce the environmental impact through phosphorous and nitrogen release reduction, e.g. in pigs (such as described in Golovan S P, et al., Nat Biotechnol. 2001; 19:741-5; Zhang X et al., elife. 2018). shRNA decoy may be introduced to induce avian influenza resilience e.g. in chicken (such as described in Lyall et al., Science. 2011; 331:223-6). Lysozyme or lysostaphin may be introduced to induce mastitis resilience e.g., in goat and cow (such as described in Maga E A et al., Foodborne Pathog Dis. 2006; 3:384-92; Wall R J, et al., Nat Biotechnol. 2005; 23:445-51). Histone deacetylase such as HDAC6 may be introduced to induce PRRSV resilience, e.g., in pig (such as described in Lu T., et al., PLOS One. 2017; 12: e0169317). CD163 may be modified (e.g., inactivated or removed) to introduce PRRSV resilience in pigs (such as described in Prather R S et al., Sci Rep. 2017 Oct. 17; 7 (1): 13371). Similar approaches may be used to inhibit or remove viruses and bacteria (e.g., Swine Influenza Virus (SIV) strains which include influenza C and the subtypes of influenza A known as H1N1, H1N2, H2N1, H3N1, H3N2, and H2N3, as well as pneumonia, meningitis and oedema) that may be transmitted from animals to humans.

In one embodiment, one or more genes may be modified or edited for disease resistance and production traits. Myostatin (e.g., GDF8) may be modified to increase muscle growth, e.g., in cow, sheep, goat, catfish, and pig (such as described in Crispo M et al., PLOS One. 2015; 10: e0136690; Wang X, et al., Anim Genet. 2018; 49:43-51; Khalil K, et al., Sci Rep. 2017; 7:7301; Kang J-D, et al., RSC Adv. 2017; 7:12541-9). Pc POLLED may be modified to induce horlessness, e.g., in cow (such as described in Carlson D F et al., Nat Biotechnol. 2016; 34:479-81). KISSIR may be modified to induce boretaint (hormone release during sexual maturity leading to undesired meat taste), e.g., in pigs. Dead end protein (dnd) may be modified to induce sterility, e.g., in salmon (such as described in Wargelius A, et al., Sci Rep. 2016; 6:21284). Nano2 and DDX may be modified to induce sterility (e.g., in surrogate hosts), e.g., in pigs and chicken (such as described Park K-E, et al., Sci Rep. 2017; 7:40176; Taylor L et al., Development. 2017; 144:928-34). CD163 may be modified to induce PRRSV resistance, e.g., in pigs (such as described in Whitworth K M, et al., Nat Biotechnol. 2015; 34:20-2). RELA may be modified to induce ASFV resilience, e.g., in pigs (such as described in Lillico S G, et al., Sci Rep. 2016; 6:21645). CD18 may be modified to induce Mannheimia (Pasteurella) haemolytica resilience, e.g., in cows (such as described in Shanthalingam S, et al., roc Natl Acad Sci USA. 2016; 113:13186-90). NRAMPI may be modified to induce tuberculosis resilience, e.g., in cows (such as described in Gao Y et al., Genome Biol. 2017; 18:13). Endogenous retrovirus genes may be modified or removed for xenotransplantation such as described in Yang L, et al. Science. 2015; 350:1101-4; Niu D et al., Science. 2017; 357:1303-7). Negative regulators of muscle mass (e.g., Myostatin) may be modified (e.g., inactivated) to increase muscle mass, e.g., in dogs (as described in Zou Q et al., J Mol Cell Biol. 2015 December; 7 (6): 580-3).

Animals such as pigs with severe combined immunodeficiency (SCID) may generated (e.g., by modifying RAG2) to provide useful models for regenerative medicine, xenotransplantation (discussed also elsewhere herein), and tumor development. Examples of methods and approaches include those described Lee K, et al., Proc Natl Acad Sci USA. 2014 May 20; 111 (20): 7260-5; and Schomberg et al. FASEB Journal, April 2016; 30 (1): Suppl 571.1.

SNPs in the animals may be modified. Examples of methods and approaches include those described Tan W. et al., Proc Natl Acad Sci USA. 2013 Oct. 8; 110 (41): 16526-31; Mali P, et al., Science. 2013 Feb. 15; 339 (6121): 823-6.

Stem cells (e.g., induced pluripotent stem cells) may be modified and differentiated into desired progeny cells, e.g., as described in Heo Y T et al., Stem Cells Dev. 2015 Feb. 1; 24 (3): 393-402.

Profile analysis (such as Igenity) may be performed on animals to screen and identify genetic variations related to economic traits. The genetic variations may be modified to introduce or improve the traits, such as carcass composition, carcass quality, maternal and reproductive traits and average daily gain.

### Kits

In one aspect, the invention provides kits containing any one or more of the elements disclosed in the above methods and compositions. In one aspect, the invention provides a kit comprising one or more of the components described herein. In one embodiment, the kit comprises the compositions herein and instructions for using the kit. In one embodiment, the kit comprises a vector system and instructions for using the kit. In one embodiment, the kit comprises a delivery system and instructions for using the kit. In one embodiment, the kit comprises a vector system and instructions for using the kit. Elements may be provided individually or in combinations, and may be provided in any suitable container, such as a vial, a bottle, or a tube. The kits may include the ωRNA and optionally an unbound protector strand as described herein. The kits may include the ωRNA with a protector strand bound to at least partially to a reprogrammable spacer portion of the ωRNA sequence (i.e., phRNA). Thus the kits may include the phRNA in the form of a partially double stranded nucleotide sequence as described here. In one embodiment, the kit includes instructions in one or more languages, for example in more than one language. The instructions may be specific to the applications and methods described herein.

In one embodiment, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g., in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In one embodiment, the buffer is alkaline. In one embodiment, the buffer has a pH from about 7 to about 10. In one embodiment, the kit comprises one or more oligonucleotides corresponding to a ωRNA scaffold, reprogrammable sequence for insertion into a vector so as to operably link the ωRNA sequence and a regulatory element. In one embodiment, the kit comprises a homologous recombination template polynucleotide. In one embodiment, the kit comprises one or more of the vectors and/or one or more of the polynucleotides described herein. The kit may advantageously allow to provide all elements of the systems of the invention.

The invention is further described in the following examples, which do not limit the scope of the invention described in the claims.

Further embodiments are illustrated in the following Examples which are given for illustrative purposes only and are not intended to limit the scope of the invention.

### EXAMPLES

### Example 1

The widespread IS200/605 transposon family encodes diverse re-targetable RNA-guided endonucleasesThe prokaryotic RNA-guided defense system CRISPR-Cas9 (type II CRISPR-Cas), which has been adopted for genome editing in eukaryotic cells (Zhang, F. (2019), Quarterly Reviews of Biophysics 52; Hille, F, et al. (2018), Cell 172:1239-1259), is thought to have evolved from IscB proteins. Despite its wide distribution across prokaryotes and shared domain composition and architecture with Cas9, the function of IscB remains unknown (FIG. 43). Moreover, given that IscB has not been reported to be associated with non-coding RNA (ncRNA) or CRISPR arrays, the evolutionary origins of the RNA-guided activity in Cas9 systems are unclear. IscB is encoded by a distinct subset of IS200/605 superfamily transposons that also include transposons encoding tnpB, a putative endonuclease distantly related to iscB and thought to be the ancestor of Cas12, the type V CRISPR effector (Kapitonav, V. et al. (2015), J. Bacteriol. 198, 797-807; Siguir, P. et al. (2014), FEMS Microbiol. Rev. 38, 865-891; S. Shmakov, S. et al. (2017), Nat. Rev. Microbiol. 15, 169-182). Using phylogenetic analysis, RNA-seq, and biochemical experiments, Applicants sought to elucidate the functions of these proteins and the origin of RNA-guided activity in class 2 CRISPR systems.

### Evolution and Diversity of IscB Systems

Applicants next investigated the evolutionary relationships between IscB, Cas9, and other homologous proteins to gain a broader insight into the evolution of RNA-guided mechanisms. In our search for proteins containing split RuvC domains, Applicants detected another group of shorter, ˜350 aa IscB homologs that are also encoded in IS200/605 superfamily transposons. These proteins contain a PLMP domain and split RuvC but lack the HNH domain. Applicants renamed these proteins IsrB (Insertion sequence RuvC-like OrfB) to emphasize their distinct domain architecture, replacing the previous designation, IscB1 (Kapitonov, V. et al. (2015), J. Bacteriol. 198, 797-807). In addition to IscB and IsrB, Applicants identified a family of even smaller (˜180 aa) proteins that only contained the PLMP domain and HNH domain but no RuvC domain, which Applicants named IshB (Insertion sequence HNH-like OrfB).

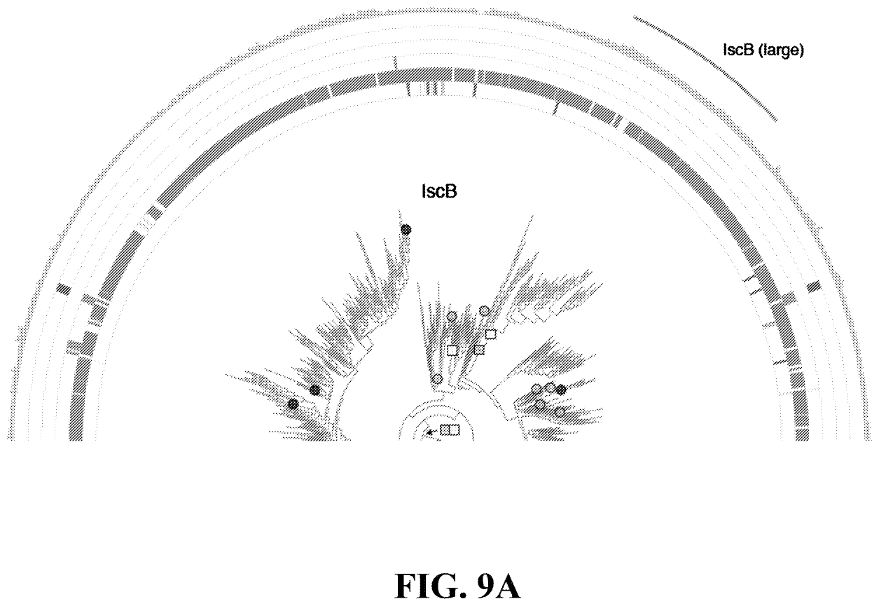

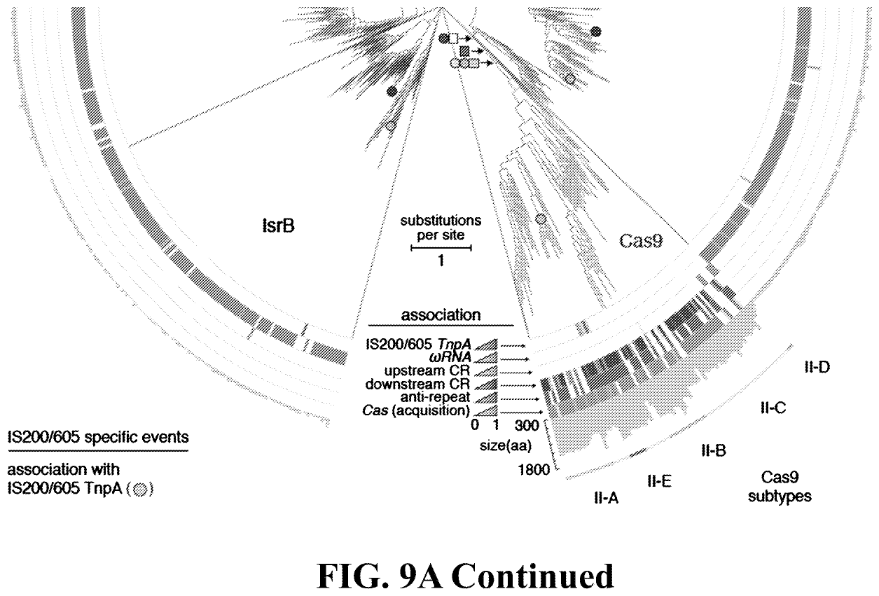

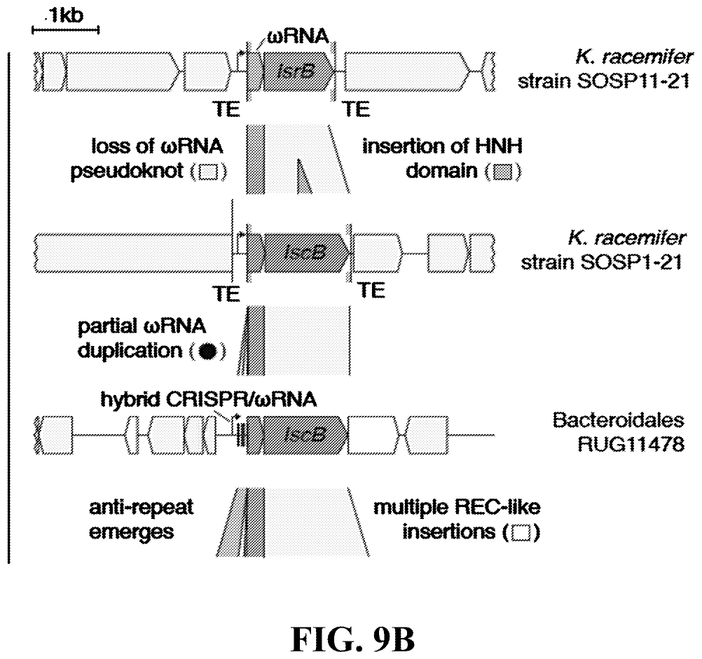

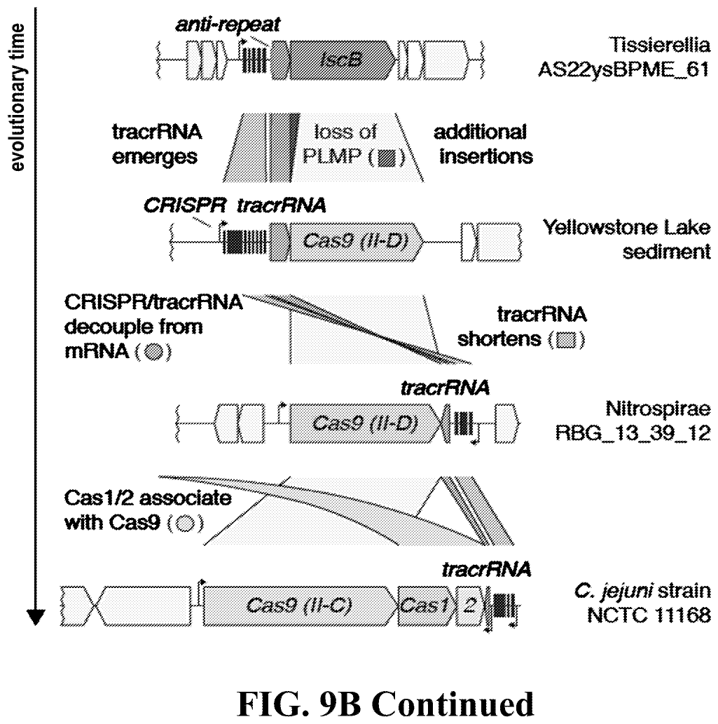

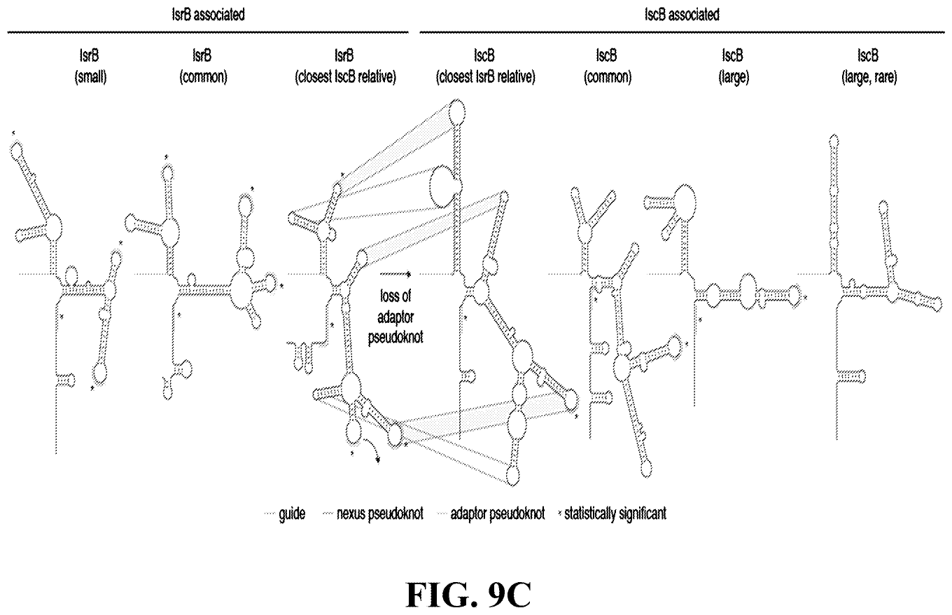

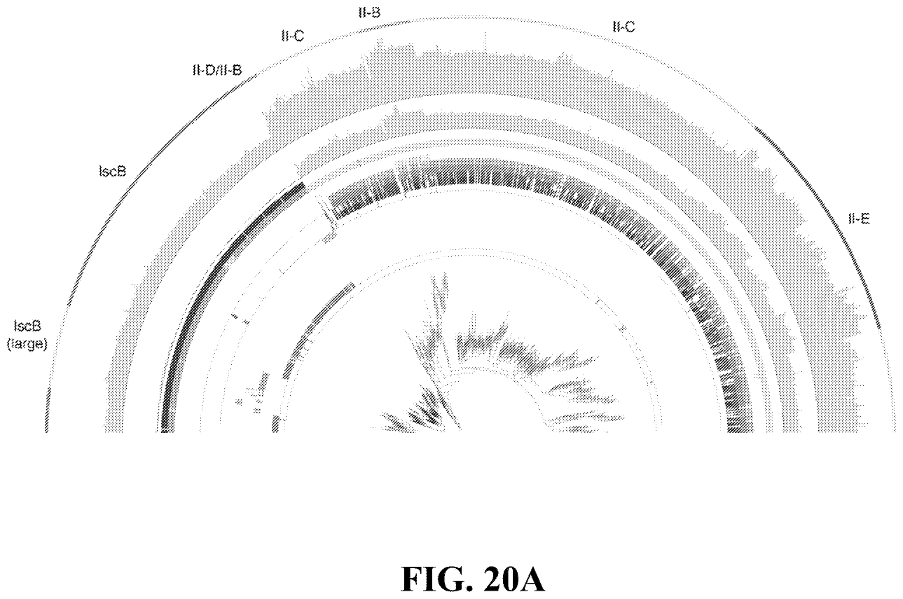

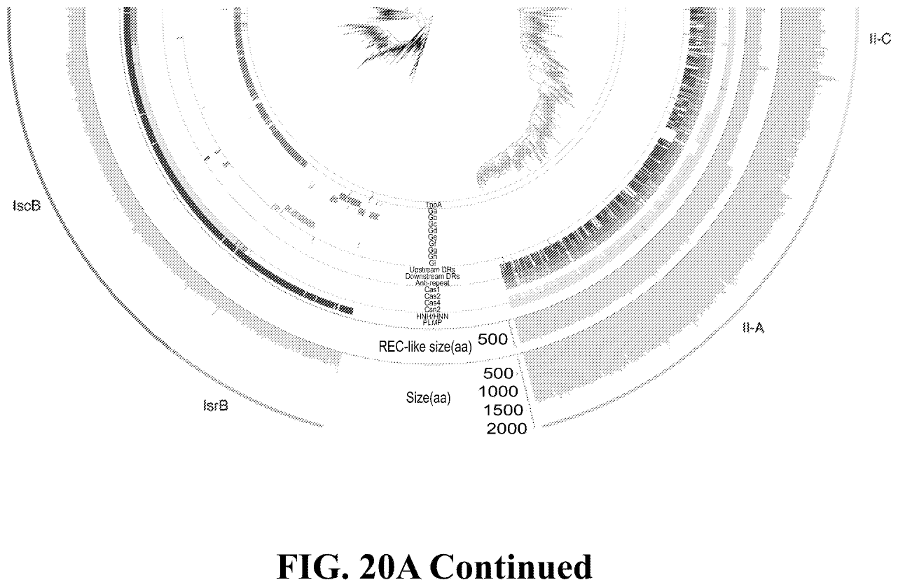

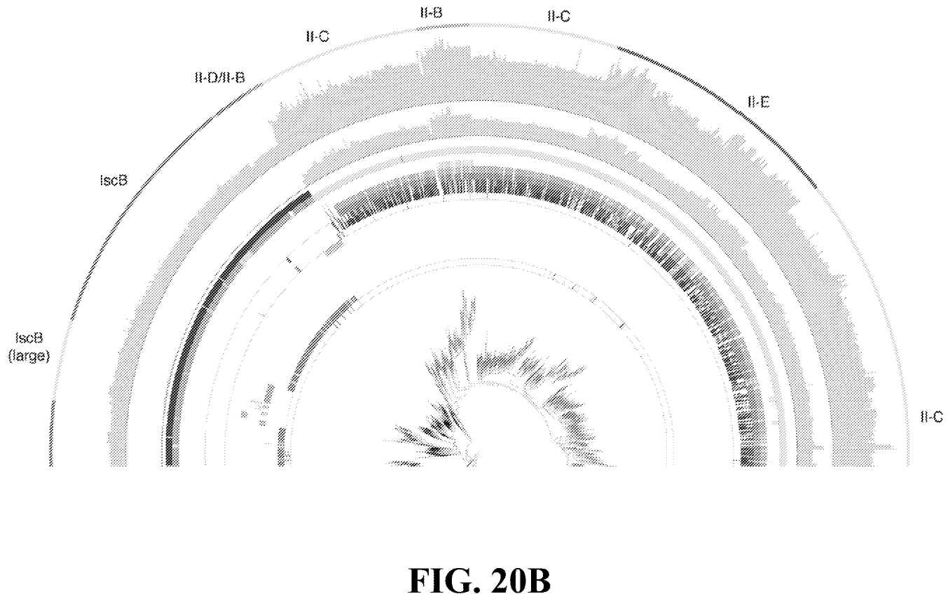

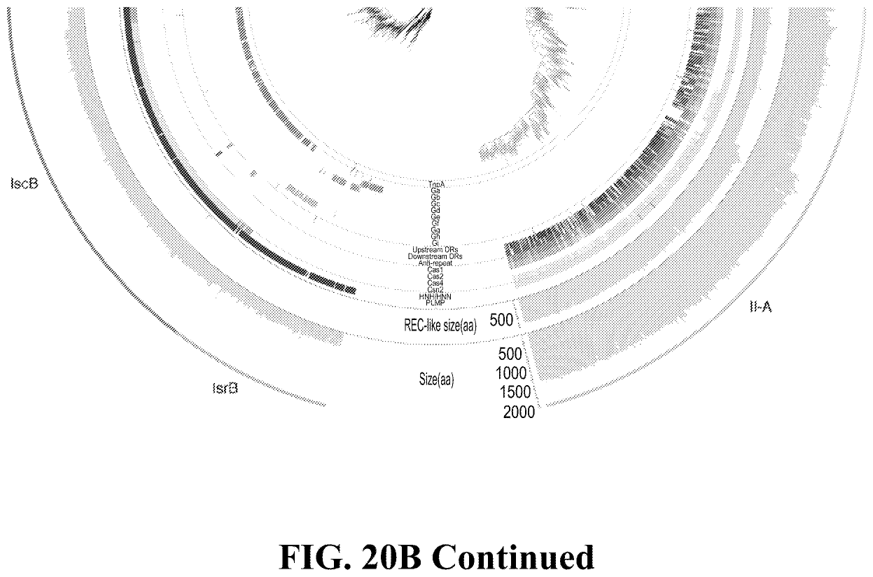

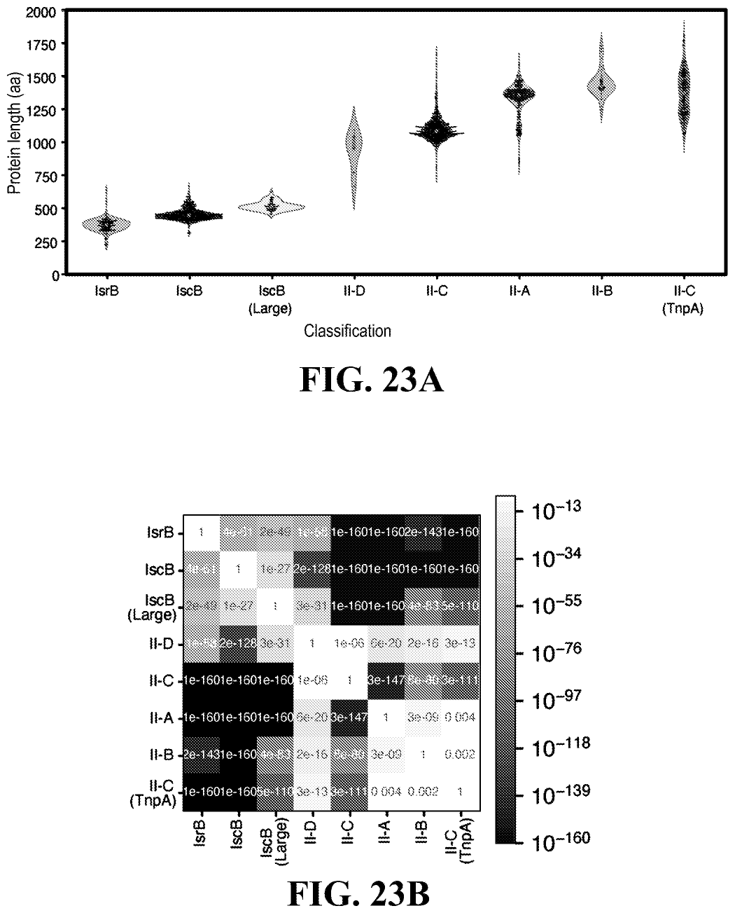

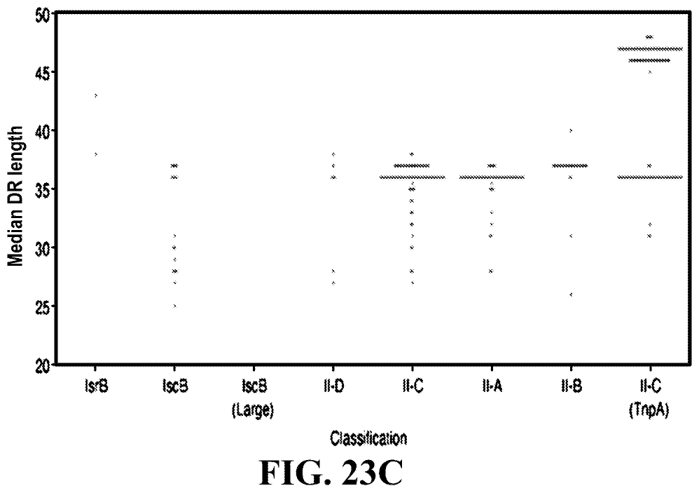

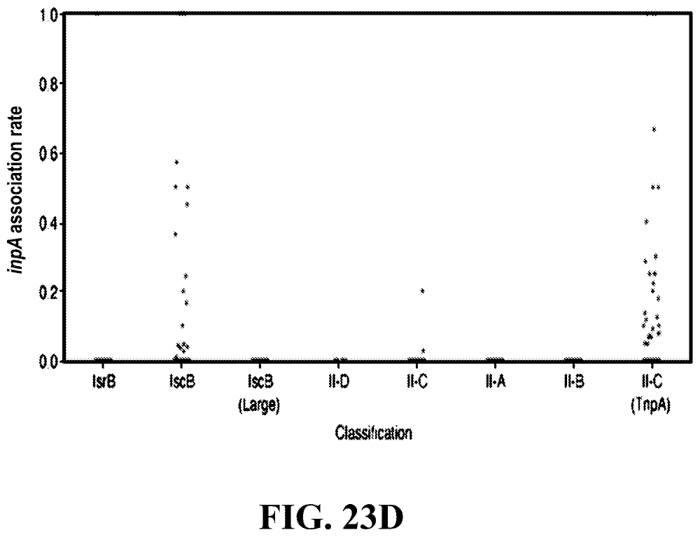

To investigate the relationships between these proteins, Applicants built a maximum likelihood (ML) tree from a multiple alignment of the split RuvC nuclease and BH domains using IQ-TREE 2 (FIGS. 9A, 20) (Mihn, B. et al (2020), Mol. Biol. Evol. 37, 1530-1534). The topology of the resulting tree was supported by several additional ML and Bayesian phylogenetic and robustness analyses (FIGS. 10, 20, 21, 22), see Supplementary Text for details). In the resulting tree, IsrB, IscB, and Cas9 formed distinct, strongly supported clades, suggesting that each of these nucleases originated from a unique evolutionary event (FIGS. 9A, 10, 21C-21D, and Supplementary Text). Applicants then analyzed the associations between each protein cluster and IS200/605 tnpA genes (Kapitonov, V. et al. (2015), J. Bacteriol. 198, 797-807), ωRNAs, CRISPR-Cas adaptation genes (cas1, cas2, cas4, and csn2), CRISPR arrays upstream and downstream of the respective ORF, and CRISPR anti-repeats (FIG. 9A). As discussed above, IscB and isrB were rarely associated with CRISPR arrays and were not found to be associated with CRISPR-Cas adaptation genes. The isrBs are associated with structurally distinct ωRNAs. The iscBs are flanked by transposon ends similar to those mobilized by TnpA (Kapitonov, V. et al. (2015), J. Bacteriol. 198, 797-807), but are only found near tnpA in 56/2811 of unique IscB loci (2.0%) (FIGS. 9A, 23D).

Additionally, Applicants identified two distinct groups of Cas9s. The first is a new subtype, II-D, a group of relatively small cas9s (˜700aa) that are not associated with any other known cas genes (Makarova, K., et al. (2020), Nat. Rev. Microbiol. 18, 67-83). The second is a distinct clade branching from within the II-C subtype, which includes exceptionally large cas9s (>1700aa) that are associated with tnpA (FIG. 9A, FIG. 23). The tnpA-associated II-C loci often encompass unusually long DRs (more than 42 bp in length) and in some cases encode HIRAN domain proteins between the cas9 and other cas genes (FIG. 9A). Predicted transposon ends surround various combinations of the tnpA, cas acquisition genes, and CRISPR arrays in these loci.

These phylogenetic and association analyses confirm that IS200/605 transposon-encoded IscBs and IsrBs share a common evolutionary history with Cas9 (Supplementary Text). Given the deep position of the IsrB clade in the tree (FIG. 9A) and the lack of the HNH domain, IsrBs likely represent the ancestral state, probably having evolved from the compact RuvC endonuclease (Majorek, K. et al. (2014), Nucleic Acids Res. 42, 4160-4179). Almost all isrBs are associated with an ωRNA, suggesting that these systems became RNA-guided at an early stage of evolution, concomitantly with the insertions in the RuvC-like domain that are likely to be involved in complex formation with ωRNA. IsrB subsequently gained the HNH domain, possibly through insertion of another mobile element or recombination with a gene encoding an IshB-like protein, founding the IscB family (gray squares, FIG. 9A-9B, Supplementary Text).

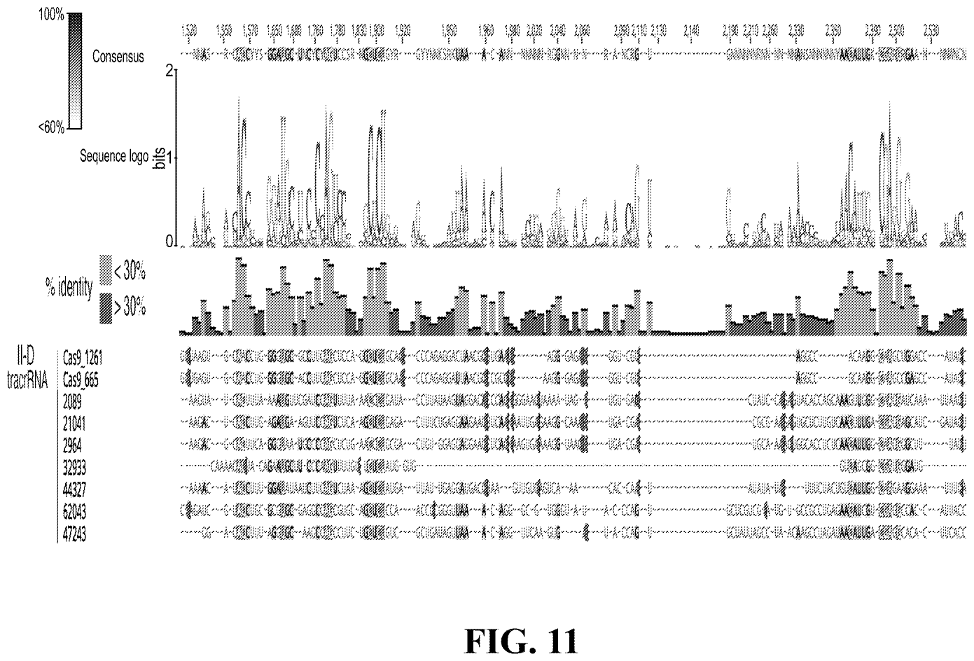

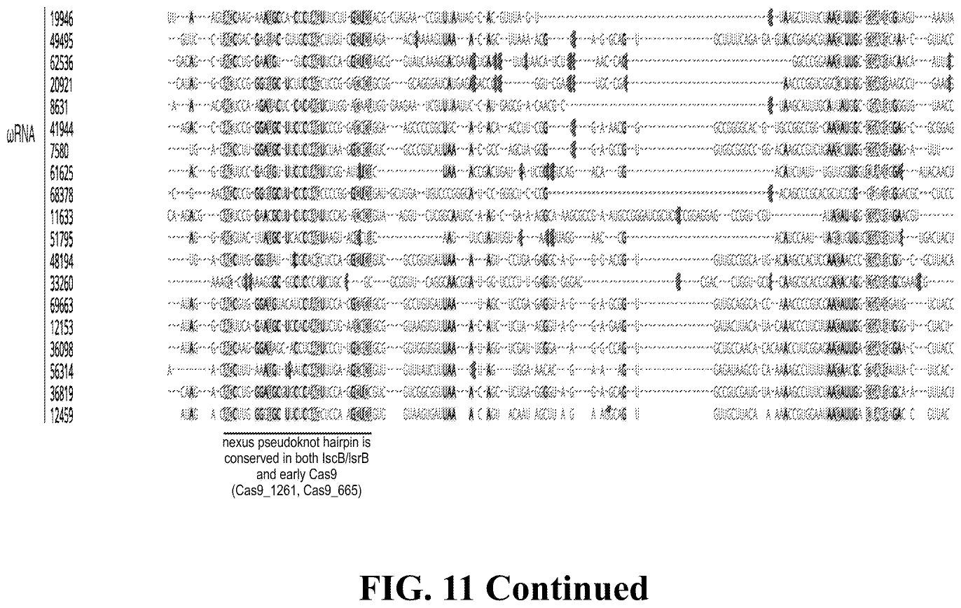

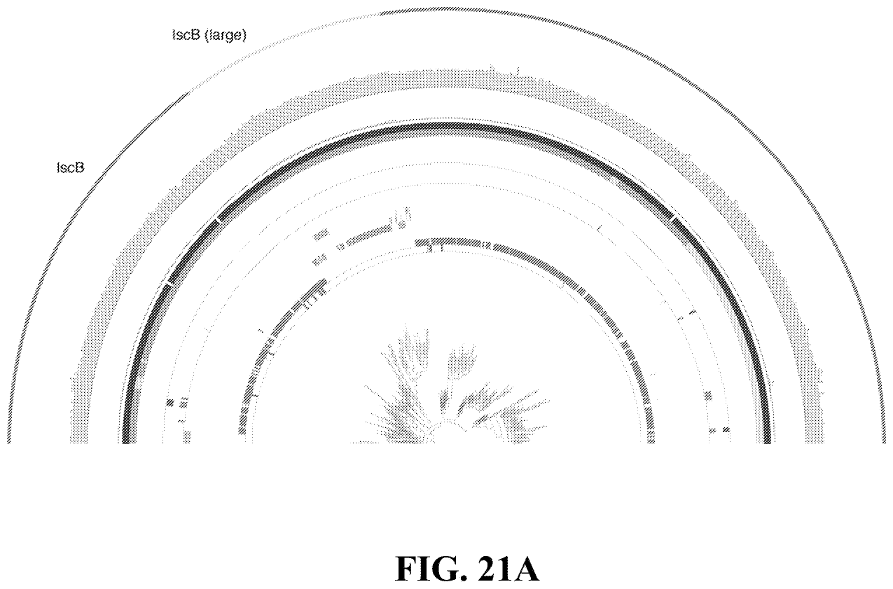

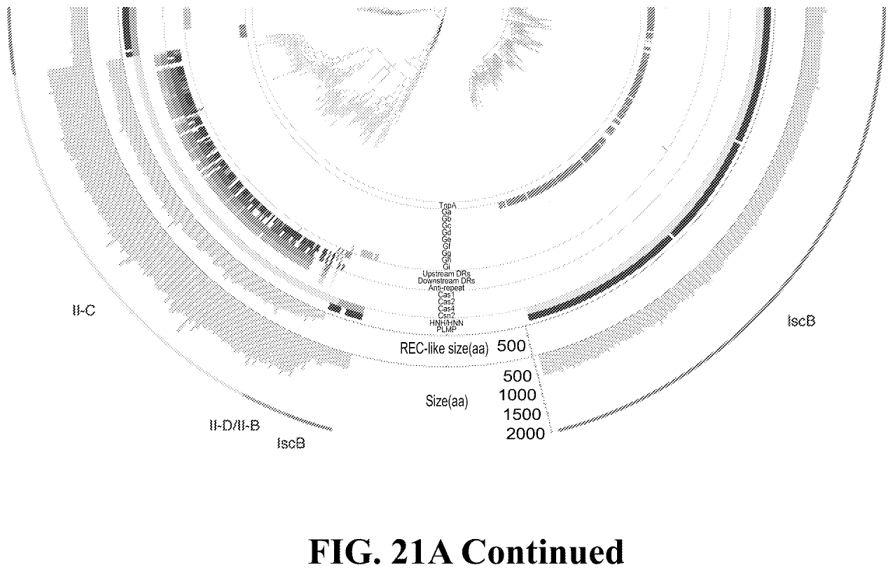

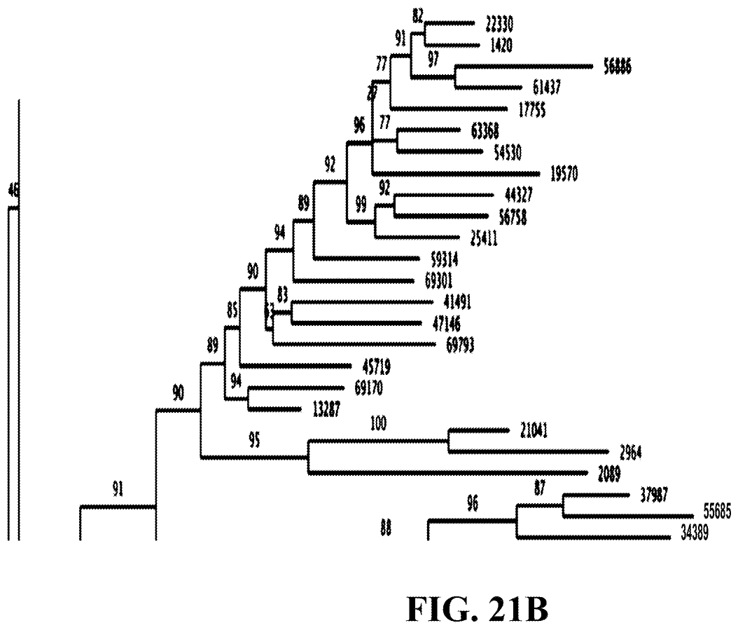

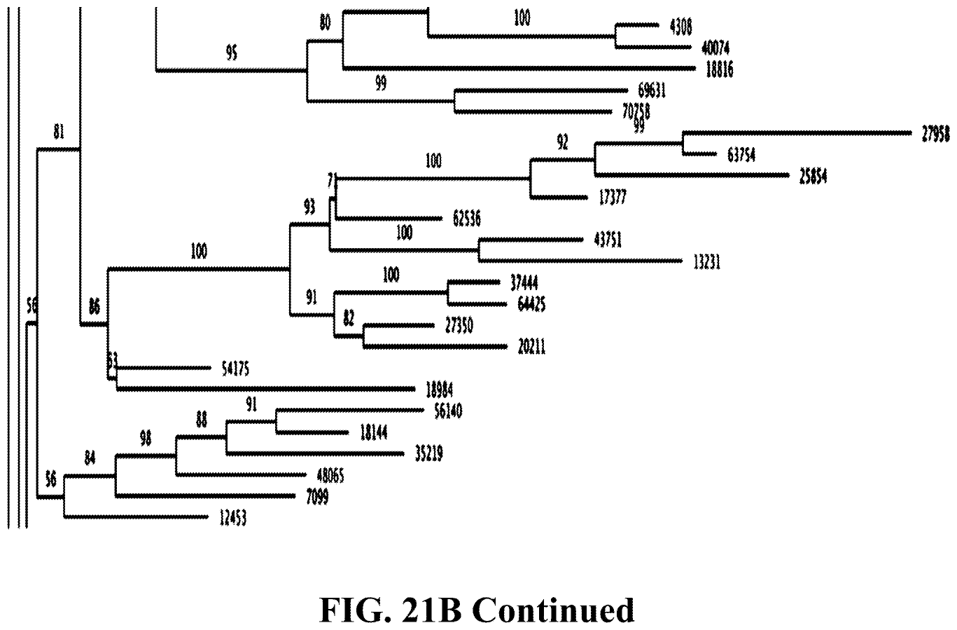

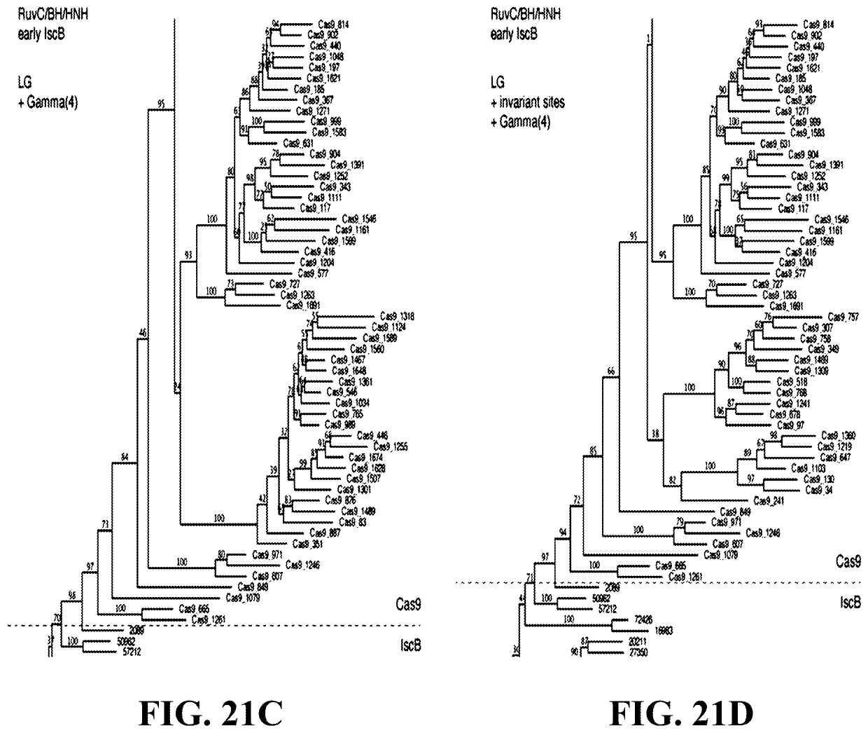

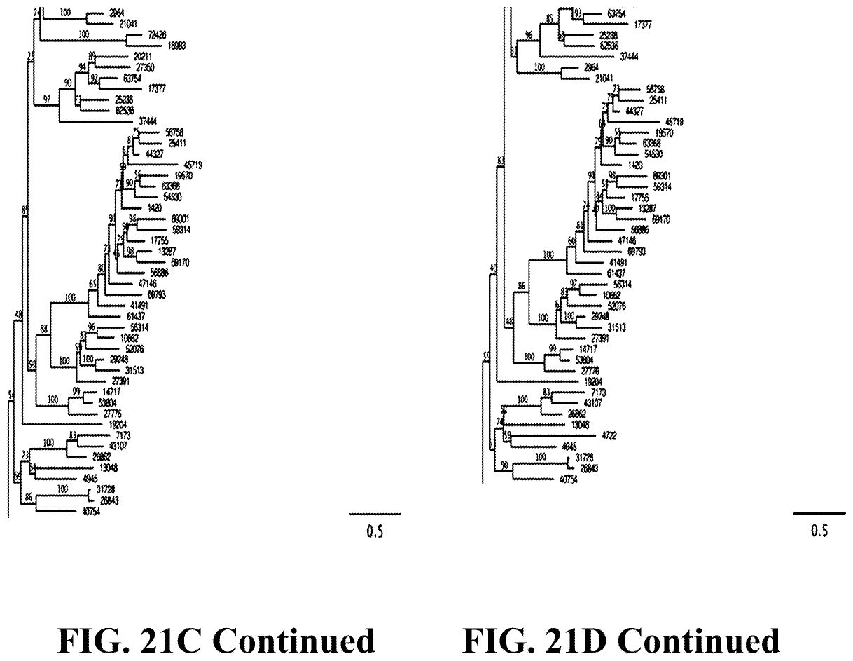

CRISPR arrays emerged within IscB systems on multiple, independent occasions (black circles, FIG. 9A-9B). These short arrays consist of repeats that could have evolved by duplication of segments of the ancestral ωRNA. The resulting systems encompass a hybrid CRISPR-ωRNA that consists of a CRISPR array preceding a partial ωRNA. These CRISPR-associated IscB proteins likely also gained REC-like insertions between the RuvC-I and RuvC-II subdomains on a number of occasions, often contemporaneously with or shortly after the CRISPR association (white squares, FIG. 9A-9B). In particular, one CRISPR-associated IscB cluster (cluster 2089) apparently founded the Cas9 family (FIG. 10) upon the loss of the hallmark PLMP domain (gray square, FIG. 9A-9B). Moreover, the tracrRNAs of Subtype II-D, a deep branch in the Cas9 subtree (ML branch support: ≥97/100, Bayesian posterior probability: 100%, FIG. 21B-D, FIG. 10), shows significant similarity to IscB ωRNAs (E-value 4.1e-8), suggesting that the Cas9 tracrRNA originally evolved from ωRNA (FIG. 11). The continued evolution of Cas9 apparently involved the gain of additional REC-like insertions between the bridge helix and the RuvC-II domains resulting in increased protein size. Finally, upon the association with the CRISPR adaptation machinery (cas1, cas2, and possibly cas4) (light gray circles, FIG. 9A-B), a burst of Cas9 diversification and widespread dispersion among bacteria via horizontal gene transfer followed, resulting in the evolution of multiple type II CRISPR subtypes.

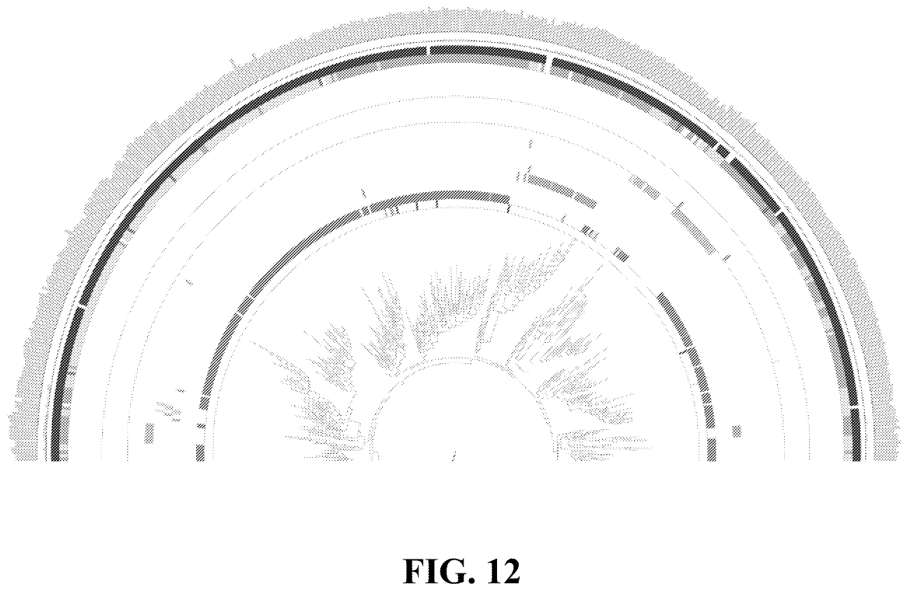

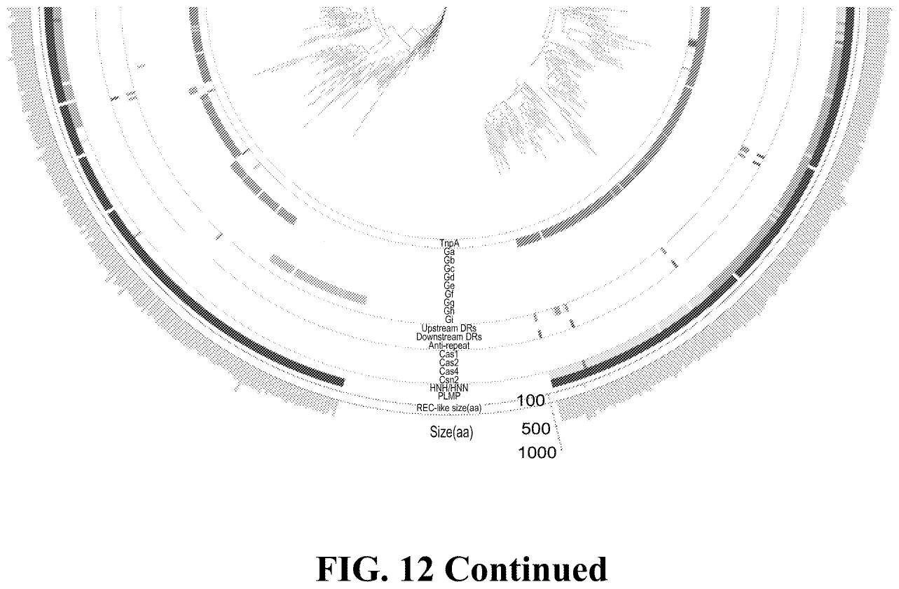

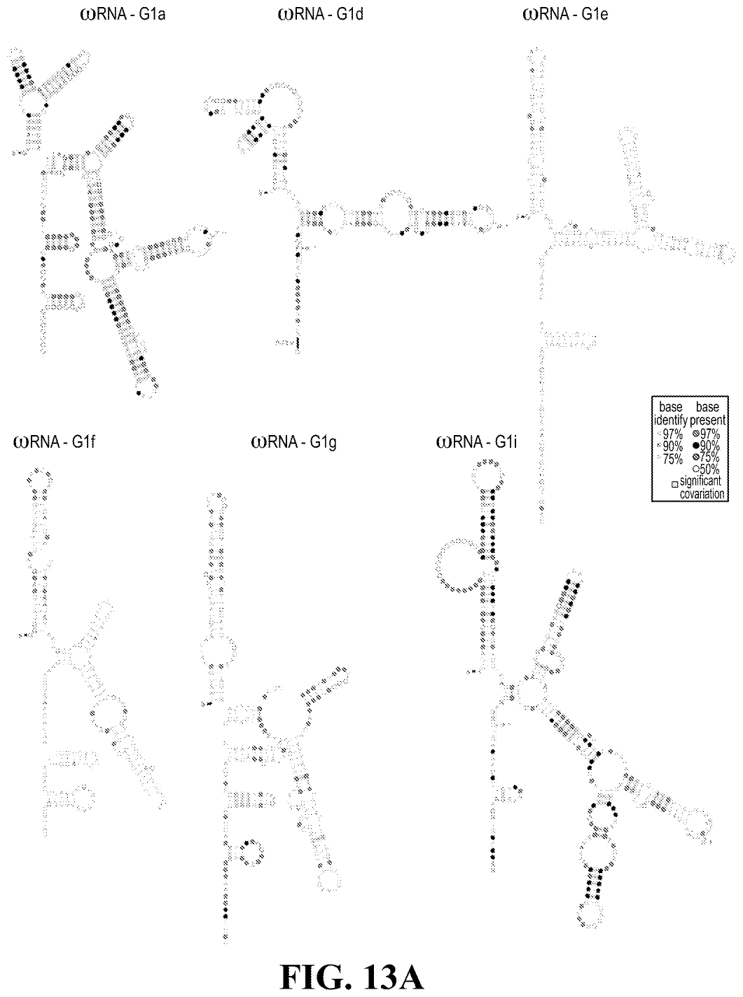

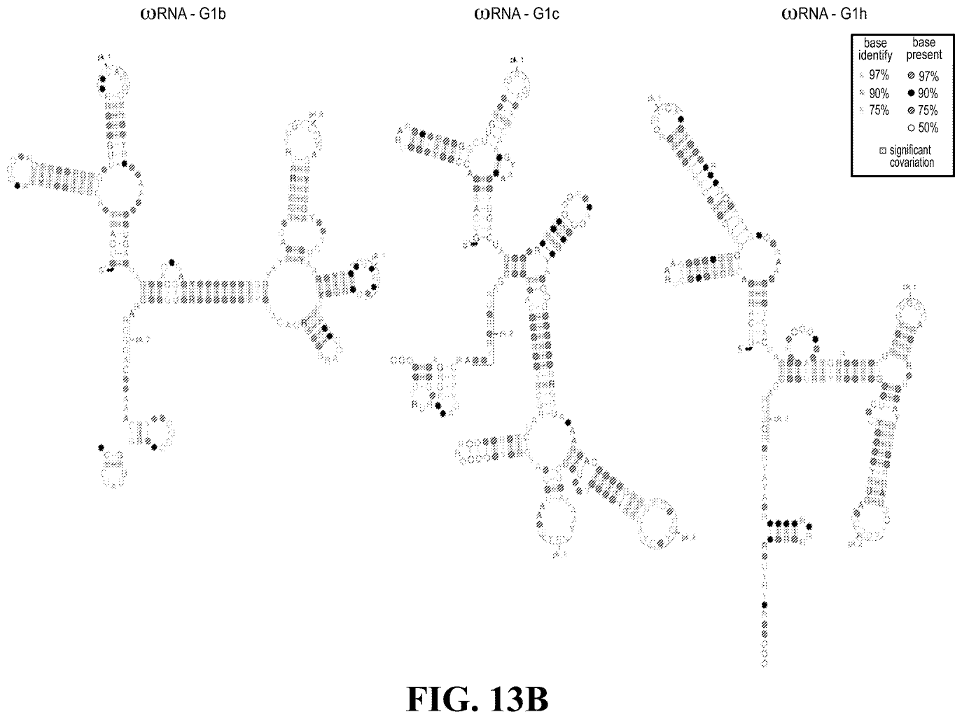

Applicants also explored the evolutionary history of ωRNAs. By iteratively building a set of ωRNA profiles that spanned all major groups of ωRNAs associated with iscBs and isrBs, Applicants found that diverse ωRNAs are associated with almost all iscBs and isrBs. Moreover, different IsrB and IscB clades are associated with distinct ωRNA structures (FIG. 12, FIG. 13A, FIG. 20A). The transition from isrB to iscB was likely accompanied by loss of a second pseudoknot, the adaptor pseudoknot, between the transposon end region and the multi-stem loop in isrB-associated ωRNAs (light gray square, FIG. 9A-9C). The inverse relationship between the complexity of the ωRNA structure and the associated protein size is also reflected by the simplified ωRNA structures associated with clades of large IscBs and the even smaller tracrRNAs associated with large Cas9s (FIG. 9C, FIG. 13). Accession numbers and natural targets search data are shown in Table 6A-6H.

TABLE 6
Table 6A-6H. Accession numbers and natural target search data.
A. Early Cas9 Amino Acid
N sites 1048
N proteins 128

| Model | −LnL | df | AIC | AICc | BIC |
|---|---|---|---|---|---|
| LG | 158546.793 | 253 | 317599.586 | 317761.456 | 318853.11 |
| LG + I | 158165.737 | 254 | 316839.474 | 317002.828 | 318097.952 |
| LG + G4 | 153534.28 | 254 | 307576.559 | 307739.914 | 308835.037 |
| LG + I + G4 | 153450.975 | 255 | 307411.95 | 307576.798 | 308675.383 |
| LG + F + I + G4 | 153344.18 | 274 | 307236.361 | 307431.316 | 308593.932 |
| WAG + I + G4 | 153438.079 | 255 | 307386.158 | 307551.006 | 308649.59 |
| WAG + F + I + G4 | 153281.497 | 274 | 307110.994 | 307305.949 | 308468.565 |
| JTT + I + G4 | 154955.321 | 255 | 310420.642 | 310585.491 | 311684.075 |
| JTT + F + I + G4 | 154753.553 | 274 | 310055.105 | 310250.06 | 311412.676 |
| JTTDCMut + I + G4 | 154922.992 | 255 | 310355.983 | 310520.832 | 311619.416 |
| JTTDCMut + F + I + G4 | 154725.109 | 274 | 309998.219 | 310193.173 | 311355.79 |
| DCMut + I + G4 | 155870.3 | 255 | 312250.6 | 312415.448 | 313514.032 |
| DCMut + F + I + G4 | 155113.884 | 274 | 310775.767 | 310970.722 | 312133.338 |
| VT + I + G4 | 153592.848 | 255 | 307695.696 | 307860.544 | 308959.128 |
| VT + F + I + G4 | 153448.997 | 274 | 307445.995 | 307640.95 | 308803.566 |
| PMB + I + G4 | 154434.683 | 255 | 309379.365 | 309544.214 | 310642.798 |
| PMB + F + I + G4 | 154055.781 | 274 | 308659.563 | 308854.517 | 310017.134 |
| Blosum62 + I + G4 | 154250.423 | 255 | 309010.846 | 309175.695 | 310274.279 |
| Blosum62 + F + I + G4 | 153937.508 | 274 | 308423.015 | 308617.97 | 309780.586 |
| Dayhoff + I + G4 | 155873.824 | 255 | 312257.649 | 312422.497 | 313521.082 |
| Dayhoff + F + I + G4 | 155119.656 | 274 | 310787.312 | 310982.267 | 312144.883 |
| mtREV + I + G4 | 165269.973 | 255 | 331049.945 | 331214.794 | 332313.378 |
| mtREV + F + I + G4 | 157064.584 | 274 | 314677.168 | 314872.123 | 316034.739 |
| mtART + I + G4 | 169405.3 | 255 | 339320.601 | 339485.449 | 340584.034 |
| mtART + F + I + G4 | 159787.176 | 274 | 320122.351 | 320317.306 | 321479.923 |
| mtZOA + I + G4 | 163921.253 | 255 | 328352.507 | 328517.355 | 329615.94 |
| mtZOA + F + I + G4 | 157454.96 | 274 | 315457.92 | 315652.874 | 316815.491 |
| mtMet + I + G4 | 166109.582 | 255 | 332729.164 | 332894.013 | 333992.597 |
| mtMet + F + I + G4 | 157397.596 | 274 | 315343.191 | 315538.146 | 316700.762 |
| mtVer + I + G4 | 167284.318 | 255 | 335078.637 | 335243.485 | 336342.07 |
| mtVer + F + I + G4 | 159774.207 | 274 | 320096.413 | 320291.368 | 321453.984 |
| mtInv + I + G4 | 166773.594 | 255 | 334057.187 | 334222.036 | 335320.62 |
| mtInv + F + I + G4 | 156166.225 | 274 | 312880.451 | 313075.405 | 314238.022 |
| mtMAM + I + G4 | 170224.016 | 255 | 340958.033 | 341122.881 | 342221.466 |
| mtMAM + F + I + G4 | 161394.78 | 274 | 323337.56 | 323532.515 | 324695.131 |
| HIVb + I + G4 | 158770.796 | 255 | 318051.592 | 318216.441 | 319315.025 |
| HIVb + F + I + G4 | 158521.047 | 274 | 317590.094 | 317785.049 | 318947.665 |
| HIVw + I + G4 | 165646.002 | 255 | 331802.005 | 331966.853 | 333065.438 |
| HIVw + F + I + G4 | 162883.131 | 274 | 326314.262 | 326509.217 | 327671.833 |
| FLU + I + G4 | 158550.963 | 255 | 317611.926 | 317776.774 | 318875.359 |
| FLU + F + I + G4 | 157457.023 | 274 | 315462.047 | 315657.001 | 316819.618 |
| rtREV + I + G4 | 154713.716 | 255 | 309937.431 | 310102.28 | 311200.864 |
| rtREV + F + I + G4 | 153762.52 | 274 | 308073.04 | 308267.995 | 309430.611 |
| cpREV + I + G4 | 154803.199 | 255 | 310116.399 | 310281.247 | 311379.832 |
| cpREV + F + I + G4 | 154747.45 | 274 | 310042.901 | 310237.855 | 311400.472 |

B. Early Cas9 DNA
N sites 3039
N proteins 128

| | | | | | |
|---|---|---|---|---|---|
| GTR + F | 292394.76 | 261 | 585311.52 | 585360.769 | 586882.553 |
| GTR + F + I | 291454.796 | 262 | 583433.591 | 583483.235 | 585010.644 |
| GTR + F + G4 | 279093.787 | 262 | 558711.574 | 558761.218 | 560288.626 |
| GTR + F + I + G4 | 278956.868 | 263 | 558439.736 | 558489.777 | 560022.808 |
| SYM + I + G4 | 279352.506 | 260 | 559225.011 | 559273.867 | 560790.025 |
| TVM + F + I + G4 | 278968.492 | 262 | 558460.983 | 558510.627 | 560038.036 |
| TVMe + I + G4 | 279407.399 | 259 | 559332.797 | 559381.261 | 560891.792 |
| TIM3 + F + I + G4 | 279049.294 | 261 | 558620.588 | 558669.837 | 560191.621 |
| TIM3e + I + G4 | 279401.196 | 258 | 559318.391 | 559366.465 | 560871.367 |
| TIM2 + F + I + G4 | 279352.841 | 261 | 559227.682 | 559276.931 | 560798.715 |
| TIM2e + I + G4 | 280434.163 | 258 | 561384.325 | 561432.399 | 562937.3 |
| TIM + F + I + G4 | 279440.534 | 261 | 559403.068 | 559452.317 | 560974.101 |
| TIMe + I + G4 | 280499.184 | 258 | 561514.369 | 561562.442 | 563067.344 |
| TPM3u + F + I + G4 | 279064.274 | 260 | 558648.548 | 558697.403 | 560213.562 |
| TPM3 + F + I + G4 | 279064.274 | 260 | 558648.548 | 558697.403 | 560213.562 |
| TPM2u + F + I + G4 | 279363.223 | 260 | 559246.446 | 559295.301 | 560811.459 |
| TPM2 + F + I + G4 | 279362.542 | 260 | 559245.084 | 559293.939 | 560810.097 |
| K3Pu + F + I + G4 | 279454.965 | 260 | 559429.93 | 559478.785 | 560994.943 |
| K3P + I + G4 | 280559.351 | 257 | 561632.702 | 561680.387 | 563179.657 |
| TN + F + I + G4 | 279455.636 | 260 | 559431.272 | 559480.128 | 560996.286 |
| TNe + I + G4 | 280496.481 | 257 | 561506.961 | 561554.646 | 563053.917 |
| HKY + F + I + G4 | 279465.418 | 259 | 559448.837 | 559497.3 | 561007.831 |
| K2P + I + G4 | 280565.775 | 256 | 561643.549 | 561690.848 | 563184.486 |
| F81 + F + I + G4 | 281411.443 | 258 | 563338.885 | 563386.958 | 564891.86 |
| JC + I + G4 | 282299.492 | 255 | 565108.984 | 565155.897 | 566643.901 |

C. Omega RNA.
N site 522
N RNAs 711

| Model | −LnL | df | AIC | AICc | BIC |
|---|---|---|---|---|---|
| GTR + F | 137969.864 | 1427 | 278793.729 | 4354305.73 | 284869.42 |
| GTR + F + ASC | 137969.861 | 1427 | 278793.721 | 4354305.72 | 284869.413 |
| GTR + F + G4 | 132814.717 | 1428 | 268485.434 | 4349709.43 | 274565.383 |
| GTR + F + ASC + G4 | 132814.709 | 1428 | 268485.418 | 4349709.42 | 274565.367 |
| SYM + G4 | 132847.753 | 1425 | 268545.507 | 4332645.51 | 274612.683 |
| SYM + ASC + G4 | 132847.753 | 1425 | 268545.507 | 4332645.51 | 274612.683 |
| TVM + F + G4 | 132918.625 | 1427 | 268691.251 | 4344203.25 | 274766.942 |
| TVM + F + ASC + G4 | 132918.625 | 1427 | 268691.251 | 4344203.25 | 274766.942 |
| TVMe + G4 | 132862.651 | 1424 | 268573.302 | 4326973.3 | 274636.22 |
| TVMe + ASC + G4 | 132862.651 | 1424 | 268573.302 | 4326973.3 | 274636.22 |
| TIM3 + F + G4 | 132865.718 | 1426 | 268583.436 | 4338387.44 | 274654.87 |
| TIM3 + F + ASC + G4 | 132865.718 | 1426 | 268583.436 | 4338387.44 | 274654.87 |
| TIM3e + G4 | 132931.537 | 1423 | 268709.074 | 4321413.07 | 274767.735 |
| TIM3e + ASC + G4 | 132931.537 | 1423 | 268709.074 | 4321413.07 | 274767.735 |
| TIM2 + F + G4 | 132882.127 | 1426 | 268616.255 | 4338420.26 | 274687.689 |
| TIM2 + F + ASC + G4 | 132882.127 | 1426 | 268616.255 | 4338420.26 | 274687.689 |
| TIM2e + G4 | 132910.507 | 1423 | 268667.013 | 4321371.01 | 274725.674 |
| TIM2e + ASC + G4 | 132910.507 | 1423 | 268667.013 | 4321371.01 | 274725.674 |
| TIM + F + G4 | 132882.794 | 1426 | 268617.588 | 4338421.59 | 274689.022 |
| TIM + F + ASC + G4 | 132882.794 | 1426 | 268617.588 | 4338421.59 | 274689.022 |
| TIMe + G4 | 132943.006 | 1423 | 268732.012 | 4321436.01 | 274790.673 |
| TIMe + ASC + G4 | 132943.006 | 1423 | 268732.012 | 4321436.01 | 274790.673 |
| TPM3u + F + G4 | 132975.699 | 1425 | 268801.399 | 4332901.4 | 274868.575 |
| TPM3u + F + ASC + G4 | 132975.699 | 1425 | 268801.399 | 4332901.4 | 274868.575 |
| TPM3 + F + G4 | 132975.699 | 1425 | 268801.399 | 4332901.4 | 274868.575 |
| TPM3 + F + ASC + G4 | 132975.699 | 1425 | 268801.399 | 4332901.4 | 274868.575 |
| TPM2u + F + G4 | 132989.627 | 1425 | 268829.254 | 4332929.25 | 274896.43 |
| TPM2u + F + ASC + G4 | 132989.627 | 1425 | 268829.254 | 4332929.25 | 274896.43 |
| TPM2 + F + G4 | 132989.627 | 1425 | 268829.254 | 4332929.25 | 274896.43 |
| TPM2 + F + ASC + G4 | 132989.627 | 1425 | 268829.254 | 4332929.25 | 274896.43 |
| K3Pu + F + G4 | 132994.102 | 1425 | 268838.203 | 4332938.2 | 274905.379 |
| K3Pu + F + ASC + G4 | 132994.102 | 1425 | 268838.203 | 4332938.2 | 274905.379 |
| K3P + G4 | 132958.903 | 1422 | 268761.806 | 4315773.81 | 274816.21 |
| K3P + ASC + G4 | 132958.903 | 1422 | 268761.806 | 4315773.81 | 274816.21 |
| TN + F + G4 | 132909.484 | 1425 | 268668.968 | 4332768.97 | 274736.145 |
| TN + F + ASC + G4 | 132909.484 | 1425 | 268668.968 | 4332768.97 | 274736.145 |
| TNe + G4 | 132969.855 | 1422 | 268783.709 | 4315795.71 | 274838.113 |
| TNe + ASC + G4 | 132969.857 | 1422 | 268783.714 | 4315795.71 | 274838.117 |
| HKY + F + G4 | 133021.421 | 1424 | 268890.843 | 4327290.84 | 274953.761 |
| HKY + F + ASC + G4 | 133021.421 | 1424 | 268890.843 | 4327290.84 | 274953.761 |
| K2P + G4 | 132985.807 | 1421 | 268813.613 | 4310137.61 | 274863.759 |
| K2P + ASC + G4 | 132985.807 | 1421 | 268813.613 | 4310137.61 | 274863.759 |
| F81 + F + G4 | 135134.431 | 1423 | 273114.861 | 4325818.86 | 279173.522 |
| F81 + F + ASC + G4 | 135134.431 | 1423 | 273114.861 | 4325818.86 | 279173.522 |
| JC + G4 | 135161.994 | 1420 | 273163.988 | 4308803.99 | 279209.876 |
| JC + ASC + G4 | 135161.994 | 1420 | 273163.988 | 4308803.99 | 279209.876 |

D. PLMP Domain
N sites 49
N proteins 2050

| Model | −LnL | df | AIC | AICc | BIC |
|---|---|---|---|---|---|
| LG | 122204.458 | 4071 | 252550.916 | 33406774.9 | 260252.517 |
| LG + G4 | 118284.816 | 4072 | 244713.633 | 33415225.6 | 252417.125 |
| LG + F + G4 | 118726.584 | 4091 | 245635.168 | 33726379.2 | 253374.605 |
| WAG + G4 | 118217.215 | 4072 | 244578.43 | 33415090.4 | 252281.922 |
| WAG + F + G4 | 118513.55 | 4091 | 245209.1 | 33725953.1 | 252948.536 |
| JTT + G4 | 119140.743 | 4072 | 246425.487 | 33416937.5 | 254128.979 |
| JTT + F + G4 | 119731.465 | 4091 | 247644.929 | 33728388.9 | 255384.366 |
| JTTDCMut + G4 | 119116.725 | 4072 | 246377.451 | 33416889.5 | 254080.943 |
| JTTDCMut + F + G4 | 119688.426 | 4091 | 247558.851 | 33728302.9 | 255298.288 |
| DCMut + G4 | 120403.011 | 4072 | 248950.023 | 33419462 | 256653.515 |
| DCMut + F + G4 | 120243.902 | 4091 | 248669.803 | 33729413.8 | 256409.24 |
| VT + G4 | 117974.209 | 4072 | 244092.419 | 33414604.4 | 251795.911 |
| VT + F + G4 | 118386.151 | 4091 | 244954.302 | 33725698.3 | 252693.739 |
| PMB + G4 | 118297.184 | 4072 | 244738.368 | 33415250.4 | 252441.86 |
| PMB + F + G4 | 118922.423 | 4091 | 246026.846 | 33726770.8 | 253766.283 |
| Blosum62 + G4 | 118283.672 | 4072 | 244711.344 | 33415223.3 | 252414.836 |
| Blosum62 + F + G4 | 118856.612 | 4091 | 245895.224 | 33726639.2 | 253634.661 |
| Dayhoff + G4 | 120429.145 | 4072 | 249002.29 | 33419514.3 | 256705.782 |
| Dayhoff + F + G4 | 120262.14 | 4091 | 248706.279 | 33729450.3 | 256445.716 |
| mtREV + G4 | 127008.803 | 4072 | 262161.606 | 33432673.6 | 269865.098 |
| mtREV + F + G4 | 121622.94 | 4091 | 251427.88 | 33732171.9 | 259167.317 |
| mtART + G4 | 130752.472 | 4072 | 269648.944 | 33440160.9 | 277352.436 |
| mtART + F + G4 | 124656.265 | 4091 | 257494.53 | 33738238.5 | 265233.967 |
| mtZOA + G4 | 126309.547 | 4072 | 260763.094 | 33431275.1 | 268466.586 |
| mtZOA + F + G4 | 122039.936 | 4091 | 252261.873 | 33733005.9 | 260001.31 |
| mtMet + G4 | 127023.696 | 4072 | 262191.392 | 33432703.4 | 269894.884 |
| mtMet + F + G4 | 121736.553 | 4091 | 251655.106 | 33732399.1 | 259394.542 |
| mtVer + G4 | 129268.339 | 4072 | 266680.678 | 33437192.7 | 274384.171 |
| mtVer + F + G4 | 124100.301 | 4091 | 256382.601 | 33737126.6 | 264122.038 |
| mtInv + G4 | 126775.212 | 4072 | 261694.425 | 33432206.4 | 269397.917 |
| mtInv + F + G4 | 120370.355 | 4091 | 248922.709 | 33729666.7 | 256662.146 |
| mtMAM + G4 | 131950.378 | 4072 | 272044.757 | 33442556.8 | 279748.249 |
| mtMAM + F + G4 | 126196.539 | 4091 | 260575.079 | 33741319.1 | 268314.515 |
| HIVb + G4 | 122513.558 | 4072 | 253171.116 | 33423683.1 | 260874.608 |
| HIVb + F + G4 | 123247.507 | 4091 | 254677.014 | 33735421 | 262416.451 |
| HIVw + G4 | 127543.628 | 4072 | 263231.256 | 33433743.3 | 270934.749 |
| HIVw + F + G4 | 126951.314 | 4091 | 262084.629 | 33742828.6 | 269824.066 |
| FLU + G4 | 122300.586 | 4072 | 252745.171 | 33423257.2 | 260448.663 |
| FLU + F + G4 | 123081.967 | 4091 | 254345.934 | 33735089.9 | 262085.371 |
| rtREV + G4 | 119057.976 | 4072 | 246259.953 | 33416772 | 253963.445 |
| rtREV + F + G4 | 119052.974 | 4091 | 246287.948 | 33727031.9 | 254027.385 |
| cpREV + G4 | 119089.943 | 4072 | 246323.886 | 33416835.9 | 254027.378 |
| cpREV + F + G4 | 119416.158 | 4091 | 247014.317 | 33727758.3 | 254753.754 |

TABLE 7
A. Pertains to FIG. 13B.

| FIG. | Contig | Feature | | | |
|---|---|---|---|---|---|
| Subpanel | accession | of interest | start | end | strand |
| 31B (K. racemifer - IsrB) | NZ_ADVG01000004 | IsrB | 697010 | 698075 | 1 |
| 31B (K. racemifer - IscB) | NZ_ADVG01000003 | IscB | 1400692 | 1401982 | 1 |
| 31B (Bacteriodales RUG11478) | CACXPM010000003.1 | IscB | 91847 | 93110 | −1 |
| 31B (Tissierellia AS22ysBPME_61) | JAAZKS010000250.1 | IscB | 1343 | 2939 | −1 |
| 31B (Armatimonadota) | Ga0315277_10040887 | Cas9 | 1829 | 3848 | 1 |
| 31B (Nitrospirae RBG_13_39_12) | MHEF01000135.1 | Cas9 | 1737 | 4026 | 1 |
| 31B (C. jejuni strain NCTC 11168) | SZUC01000003.1 | Cas9 | 186809 | 189764 | −1 |

B. Pertains to FIG. 14C, H.

| FIG. | Contig | Feature | | | |
|---|---|---|---|---|---|
| Subpanel | accession | of interest | start | end | strand |
| 14C | KY407659 | IscB | 21119 | 22433 | −1 |
| 14H | NZ_ADVG01000001 | IsrB | 978948 | 980013 | −1 |

### DISCUSSION

Naturally programmable biological systems offer an efficient solution for diverse organisms to achieve scalable complexity via modularity of their components. RNA-guided defense and regulatory systems, which are widespread in prokaryotes and eukaryotes, are a prominent case in point, and have served as the basis of numerous biotechnology applications thanks to the ease with which they can be engineered and reprogrammed (Hüttenhofer, A., et al. (2006), Nat. Rev. Genet. 7, 475-482; Schneider, A. et al. (2020), EMBO Rep. 21, e51918; Koonin, E. et al. (2017), Biol. Direct. 12 e51918).

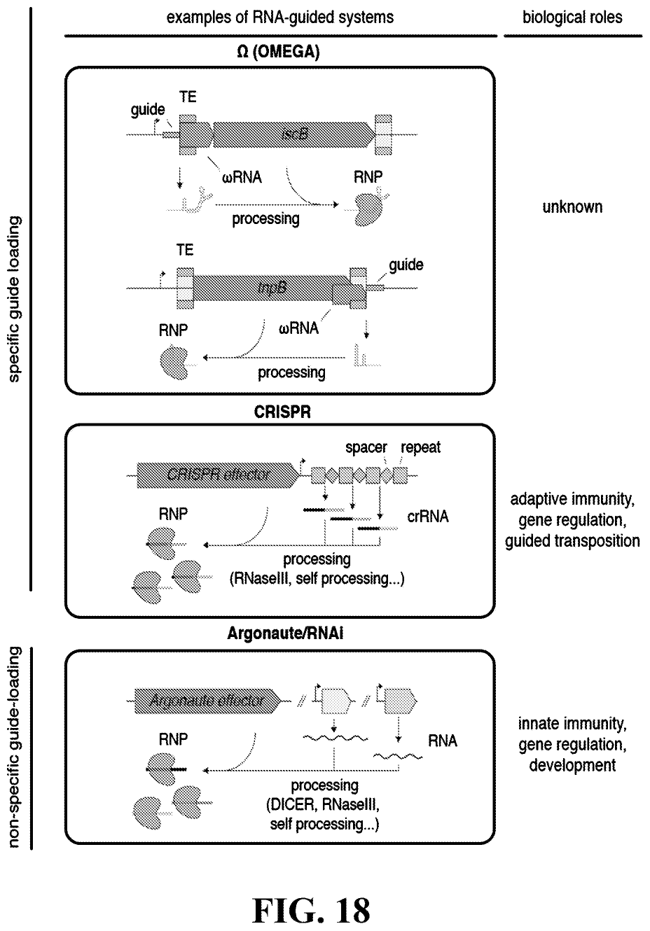

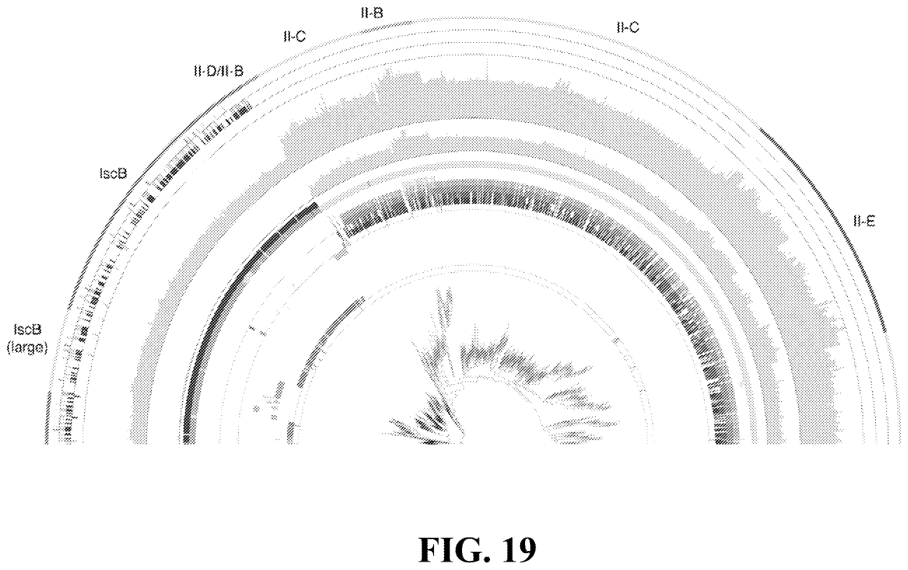

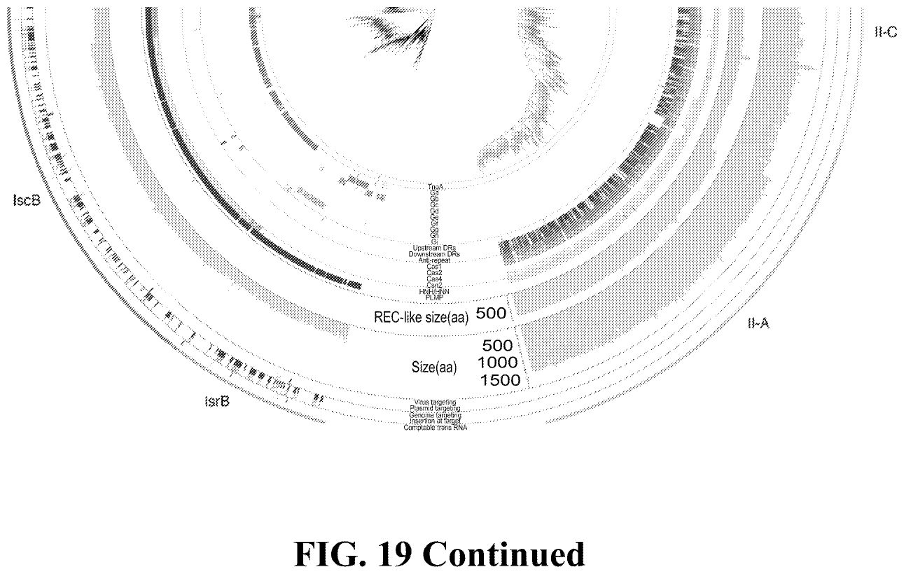

Here, through the exploration of Cas9 evolution, Applicants discovered the programmable RNA-guided mechanism of 3 highly abundant but previously uncharacterized transposon-encoded nucleases: IscB, IsrB, and TnpB, which Applicants collectively refer to as Q2 (OMEGA: Obligate Mobile Element Guided Activity) (FIG. 18) because the mobile element localization and movement likely determines the identity of their guides. Although the biological functions of 22 systems remain unknown, several hypotheses are compatible with the available evidence, including roles in facilitating TnpA-catalyzed, RNA-guided transposition, or acting as a toxin, with the transposon acting as the antitoxin, securing maintenance of IS200/605 insertions (Supplementary Text, below).

The broad distribution of the (2 systems characterized here indicates that RNA-guided mechanisms are more widespread in prokaryotes than previously suspected and suggests that RNA-guided activities are likely ancient and evolved on multiple, independent occasions, of which only the most common ones have likely been identified so far. The TnpB family is far more abundant and diverse than the IscB family; indeed, Applicants identified more than a million putative tnpB loci in bacterial and archaeal genomes, making it one of the most common prokaryotic genes altogether. These TnpBs might represent an untapped wealth of diverse RNA-guided mechanisms present not only in prokaryotes, but also in eukaryotes. Combined with our identification of a chloroplast-encoded IscB, these findings suggest that the expansion of RNA-guided systems into eukaryotic genomes could be a general phenomenon, and more broadly, that RNA-guided systems are functionally diverse and permeate all domains of life.

### REFERENCES AND NOTES

1. F. Zhang, Development of CRISPR-Cas systems for genome editing and beyond. Quarterly Reviews of Biophysics. 52 (2019).
2. F. Hille, H. Richter, S. P. Wong, M. Bratovič, S. Ressel, E. Charpentier, The biology of CRISPR-Cas: Backward and forward. Cell. 172, 1239-1259 (2018).
3. V. V. Kapitonov, K. S. Makarova, E. V. Koonin, ISC, a novel group of bacterial and Archaeal DNA transposons that encode Cas9 homologs. J. Bacteriol. 198, 797-807 (2015).
4. P. Siguier, E. Gourbeyre, M. Chandler, Bacterial insertion sequences: their genomic impact and diversity. FEMS Microbiol. Rev. 38, 865-891 (2014).
5. S. Shmakov, A. Smargon, D. Scott, D. Cox, N. Pyzocha, W. Yan, O. O. Abudayyeh, J. S. Gootenberg, K. S. Makarova, Y. I. Wolf, K. Severinov, F. Zhang, E. V. Koonin, Diversity and evolution of class 2 CRISPR-Cas systems. Nat. Rev. Microbiol. 15, 169-182 (2017).
6. B. Zetsche, J. S. Gootenberg, O. O. Abudayyeh, I. M. Slaymaker, K. S. Makarova, P. Essletzbichler, S. E. Volz, J. Joung, J. van der Oost, A. Regev, E. V. Koonin, F. Zhang, Cpf1 is a single RNA-guided endonuclease of a class 2 CRISPR-Cas system. Cell. 163, 759-771 (2015).
7. E. Deltcheva, K. Chylinski, C. M. Sharma, K. Gonzales, Y. Chao, Z. A. Pirzada, M. R. Eckert, J. Vogel, E. Charpentier, CRISPR RNA maturation by trans-encoded small RNA and host factor RNase III. Nature. 471, 602-607 (2011).
8. I. Kalvari, E. P. Nawrocki, N. Ontiveros-Palacios, J. Argasinska, K. Lamkiewicz, M. Marz, S. Griffiths-Jones, C. Toffano-Nioche, D. Gautheret, Z. Weinberg, E. Rivas, S. R. Eddy, R. D. Finn, A. Bateman, A. I. Petrov. Rfam 14: expanded coverage of metagenomic, viral and microRNA families. Nucleic Acids Res. 49, D192-D200 (2021).
9. Z. Weinberg, J. Perreault, M. M. Meyer, R. R. Breaker, Exceptional structured noncoding RNAs revealed by bacterial metagenome analysis. Nature. 462, 656-659 (2009).
10. M. Jinek, K. Chylinski, I. Fonfara, M. Hauer, J. A. Doudna, E. Charpentier, A programmable dual-RNA-guided DNA endonuclease in adaptive bacterial immunity. Science. 337, 816-821 (2012).
11. G. Gasiunas, R. Barrangou, P. Horvath, V. Siksnys, Cas9-crRNA ribonucleoprotein complex mediates specific DNA cleavage for adaptive immunity in bacteria. Proc. Natl. Acad. Sci. U.S.A 109, E2579-86 (2012).
12. G. Gasiunas, J. K. Young, T. Karvelis, D. Kazlauskas, T. Urbaitis, M. Jasnauskaite, M. M. Grusyte, S. Paulraj, P.-H. Wang, Z. Hou, S. K. Dooley, M. Cigan, C. Alarcon, N. D. Chilcoat, G. Bigelyte, J. L. Curcuru, M. Mabuchi, Z. Sun, R. T. Fuchs, E. Schildkraut, P. R. Weigele, W. E. Jack, G. B. Robb, Č. Venclovas, V. Siksnys, A catalogue of biochemically diverse CRISPR-Cas9 orthologs. Nat. Commun. 11, 5512 (2020).
13. M. Jinek, F. Jiang, D. W. Taylor, S. H. Sternberg, E. Kaya, E. Ma, C. Anders, M. Hauer, K. Zhou, S. Lin, M. Kaplan, A. T. Iavarone, E. Charpentier, E. Nogales, J. A. Doudna, Structures of Cas9 endonucleases reveal RNA-mediated conformational activation. Science. 343, 1247997 (2014).
14. B. Q. Minh, H. A. Schmidt, O. Chernomor, D. Schrempf, M. D. Woodhams, A. von Haeseler, R. Lanfear, IQ-TREE 2: New Models and Efficient Methods for Phylogenetic Inference in the Genomic Era. Mol. Biol. Evol. 37, 1530-1534 (2020).
15. K. S. Makarova, Y. I. Wolf, J. Iranzo, S. A. Shmakov, O. S. Alkhnbashi, S. J. J. Brouns, E. Charpentier, D. Cheng, D. H. Haft, P. Horvath, S. Moineau, F. J. M. Mojica, D. Scott, S. A. Shah, V. Siksnys, M. P. Terns, Č. Venclovas, M. F. White, A. F. Yakunin, W. Yan, F. Zhang, R. A. Garrett, R. Backofen, J. van der Oost, R. Barrangou, E. V. Koonin, Evolutionary classification of CRISPR-Cas systems: a burst of class 2 and derived variants. Nat. Rev. Microbiol. 18, 67-83 (2020).
16. K. A. Majorek, S. Dunin-Horkawicz, K. Steczkiewicz, A. Muszewska, M. Nowotny, K. Ginalski, J. M. Bujnicki, The RNase H-like superfamily: new members, comparative structural analysis and evolutionary classification. Nucleic Acids Res. 42, 4160-4179 (2014).
17. H. Nishimasu, F. A. Ran, P. D. Hsu, S. Konermann, S. I. Shehata, N. Dohmae, R. Ishitani, F. Zhang, O. Nureki, Crystal structure of Cas9 in complex with guide RNA and target DNA. Cell. 156, 935-949 (2014).
18. W. Bao, J. Jurka, Homologues of bacterial TnpB_IS605 are widespread in diverse eukaryotic transposable elements. Mob. DNA. 4, 12 (2013).
19. J. V. Gomes-Filho, L. S. Zaramela, V. C. da S. Italiani, N. S. Baliga, R. Z. N. Vêncio, T. Koide, Sense overlapping transcripts in IS1341-type transposase genes are functional non-coding RNAs in archaea. RNA Biol. 12, 490-500 (2015).
20. Z. Weinberg, C. E. Lunse, K. A. Corbino, T. D. Ames, J. W. Nelson, A. Roth, K. R. Perkins, M. E. Sherlock, R. R. Breaker, Detection of 224 candidate structured RNAs by comparative analysis of specific subsets of intergenic regions. Nucleic Acids Res. 45, 10811-10823 (2017).
21. A. Hüttenhofer, P. Schattner, The principles of guiding by RNA: chimeric RNA-protein enzymes. Nat. Rev. Genet. 7, 475-482 (2006).
22. A. Schneider, A short history of guide RNAs: The intricate path that led to the discovery of a basic biological concept. EMBO Rep. 21, e51918 (2020).
23. E. V. Koonin, Evolution of RNA- and DNA-guided antivirus defense systems in prokaryotes and eukaryotes: common ancestry vs convergence. Biol. Direct. 12 (2017).
24. H. Altae-Tran, S. Kannan, F. Zhang, Code and processed data for: The widespread IS200/605 transposon family encodes diverse programmable RNA-guided endonucleases, Version 1.0, Zenodo (2021); <link to be inserted after official acceptance of manuscript>.
25. C. Camacho, G. Coulouris, V. Avagyan, N. Ma, J. Papadopoulos, K. Bealer, T. L. Madden, BLAST+: architecture and applications. BMC Bioinformatics. 10, 421 (2009).
26. K. Katoh, D. M. Standley, MAFFT multiple sequence alignment software version 7: improvements in performance and usability. Mol. Biol. Evol. 30, 772-780 (2013).
27. M. Steinegger, J. Söding, MMseqs2 enables sensitive protein sequence searching for the analysis of massive data sets. Nat. Biotechnol. 35, 1026-1028 (2017).
28. M. Steinegger, M. Meier, M. Mirdita, H. Vöhringer, S. J. Haunsberger, J. Söding, HH-suite3 for fast remote homology detection and deep protein annotation. BMC Bioinformatics. 20, 473 (2019).
29. S. R. Eddy, Accelerated Profile HMM Searches. PLoS Comput. Biol. 7, e1002195 (2011).
30. S. A. Shmakov, K. S. Makarova, Y. I. Wolf, K. V. Severinov, E. V. Koonin, Systematic prediction of genes functionally linked to CRISPR-Cas systems by gene neighborhood analysis. Proc. Natl. Acad. Sci. U.S.A 115, E5307-E5316 (2018).
31. A. B. Crawley, J. R. Henriksen, R. Barrangou, CRISPRdisco: An Automated Pipeline for the Discovery and Analysis of CRISPR-Cas Systems. CRISPR J. 1, 171-181 (2018).
32. D. H. Haft, B. J. Loftus, D. L. Richardson, F. Yang, J. A. Eisen, I. T. Paulsen, O. White, TIGRFAMs: a protein family resource for the functional identification of proteins. Nucleic Acids Res. 29, 41-43 (2001).
33. A. Delcher, Improved microbial gene identification with GLIMMER. Nucleic Acids Research. 27 (1999), pp. 4636-4641.
34. S. Naser-Khdour, B. Q. Minh, W. Zhang, E. A. Stone, R. Lanfear, The Prevalence and Impact of Model Violations in Phylogenetic Analysis. Genome Biol. Evol. 11, 3341-3352 (2019).
35. S. Kalyaanamoorthy, B. Q. Minh, T. K. F. Wong, A. von Haeseler, L. S. Jermiin, ModelFinder: fast model selection for accurate phylogenetic estimates. Nat. Methods. 14, 587-589 (2017).
36. M. N. Price, P. S. Dehal, A. P. Arkin, FastTree 2—approximately maximum-likelihood trees for large alignments. PLoS One. 5, e9490 (2010).
37. G. Altekar, S. Dwarkadas, J. P. Huelsenbeck, F. Ronquist, Parallel Metropolis coupled Markov chain Monte Carlo for ian posterian phylogenetic inference. Bioinformatics. 20, 407-415 (2004).
38. A. Stamatakis, RAXML version 8: a tool for phylogenetic analysis and post-analysis of large phylogenies. Bioinformatics. 30, 1312-1313 (2014).
39. R. Lorenz, S. H. Bernhart, C. H. zu Siederdissen, H. Tafer, C. Flamm, P. F. Stadler, I. L. Hofacker, ViennaRNA Package 2.0. Algorithms Mol. Biol. 6, 1-14 (2011).
40. E. Rivas, J. Clements, S. R. Eddy, A statistical test for conserved RNA structure shows lack of evidence for structure in lncRNAs. Nat. Methods. 14, 45-48 (2017).
41. E. P. Nawrocki, S. R. Eddy, Infernal 1.1:100-fold faster RNA homology searches. Bioinformatics. 29, 2933-2935 (2013).
42. Z. Weinberg, R. R. Breaker, R2R—software to speed the depiction of aesthetic consensus RNA secondary structures. BMC Bioinformatics. 12, 3 (2011).
43. J. Mistry, S. Chuguransky, L. Williams, M. Qureshi, G. A. Salazar, E. L. L. Sonnhammer, S. C. E. Tosatto, L. Paladin, S. Raj, L. J. Richardson, R. D. Finn, A. Bateman, Pfam: The protein families database in 2021. Nucleic Acids Res. 49, D412-D419 (2021).
44. C. Bland, T. L. Ramsey, F. Sabree, M. Lowe, K. Brown, N. C. Kyrpides, P. Hugenholtz, CRISPR recognition tool (CRT): a tool for automatic detection of clustered regularly interspaced palindromic repeats. BMC Bioinformatics. 8, 209 (2007).
45. F. Asnicar, G. Weingart, T. L. Tickle, C. Huttenhower, N. Segata, Compact graphical representation of phylogenetic data and metadata with GraPhlAn. PeerJ. 3, e1029 (2015).
46. L. Maaten, G. Hinton, Visualizing Data using t-SNE. Journal of machine learning research. 9, 2579-2605 (2008).
47. P. G. Poličar, M. Stražar, B. Zupan, Embedding to reference t-SNE space addresses batch effects in single-cell classification. bioRxiv (2019), doi: 10.1101/671404.
48. P. J. Rousseeuw, Silhouettes: A graphical aid to the interpretation and validation of cluster analysis. J. Comput. Appl. Math. 20, 53-65 (1987).
49. R. J. G. B. Campello, D. Moulavi, J. Sander, in Advances in Knowledge Discovery and Data Mining (Springer Berlin Heidelberg, Berlin, Heidelberg, 2013), Lecture notes in computer science, pp. 160-172.
50. D. H. Parks, M. Imelfort, C. T. Skennerton, P. Hugenholtz, G. W. Tyson, CheckM: assessing the quality of microbial genomes recovered from isolates, single cells, and metagenomes. Genome Res. 25, 1043-1055 (2015).
51. P.-A. Chaumeil, A. J. Mussig, P. Hugenholtz, D. H. Parks, GTDB-Tk: a toolkit to classify genomes with the Genome Taxonomy Database. Bioinformatics (2019), doi: 10.1093/bioinformatics/btz848.
52. S. Frey, D. Görlich, A new set of highly efficient, tag-cleaving proteases for purifying recombinant proteins. J. Chromatogr. A. 1337, 95-105 (2014).
53. M. Martin, Cutadapt removes adapter sequences from high-throughput sequencing reads. EMBnet.journal. 17, 10-12 (2011).
54. B. Langmead, S. L. Salzberg, Fast gapped-read alignment with Bowtie 2. Nat. Methods. 9, 357-359 (2012).
55. A. M. Sriramachandran, G. Petrosino, M. Méndez-Lago, A. J. Schäfer, L. S. Batista-Nascimento, N. Zilio, H. D. Ulrich, Genome-wide Nucleotide-Resolution Mapping of DNA Replication Patterns, Single-Strand Breaks, and Lesions by GLOE-Seq. Mol. Cell. 78, 975-985.e7 (2020).
56. K. Clement, H. Rees, M. C. Canver, J. M. Gehrke, R. Farouni, J. Y. Hsu, M. A. Cole, D. R. Liu, J. K. Joung, D. E. Bauer, L. Pinello, CRISPResso2 provides accurate and rapid genome editing sequence analysis. Nat. Biotechnol. 37, 224-226 (2019).
57. A. Bankevich, S. Nurk, D. Antipov, A. A. Gurevich, M. Dvorkin, A. S. Kulikov, V. M. Lesin, S. I. Nikolenko, S. Pham, A. D. Prjibelski, A. V. Pyshkin, A. V. Sirotkin, N. Vyahhi, G. Tesler, M. A. Alekseyev, P. A. Pevzner, SPAdes: a new genome assembly algorithm and its applications to single-cell sequencing. J. Comput. Biol. 19, 455-477 (2012).
58. M. Turmel, C. Otis, C. Lemieux, Divergent copies of the large inverted repeat in the chloroplast genomes of ulvophycean green algae. Sci. Rep. 7, 994 (2017).
59. D. T. Hoang, O. Chernomor, A. von Haeseler, B. Q. Minh, L. S. Vinh, UFBoot2: Improving the Ultrafast Bootstrap Approximation. Mol. Biol. Evol. 35, 518-522 (2018).
60. P. J. Lockhart, A. W. Larkum, M. Steel, P. J. Waddell, D. Penny, Evolution of chlorophyll and bacteriochlorophyll: the problem of invariant sites in sequence analysis. Proc. Natl. Acad. Sci. U.S.A 93, 1930-1934 (1996).
61. A. Criscuolo, S. Gribaldo, BMGE (Block Mapping and Gathering with Entropy): a new software for selection of phylogenetic informative regions from multiple sequence alignments. BMC Evol. Biol. 10, 210 (2010).
62. J. Strecker, A. Ladha, Z. Gardner, J. L. Schmid-Burgk, K. S. Makarova, E. V. Koonin, F. Zhang, RNA-guided DNA insertion with CRISPR-associated transposases. Science. 365, 48-53 (2019).
63. S. E. Klompe, P. L. H. Vo, T. S. Halpin-Healy, S. H. Sternberg, Transposon-encoded CRISPR-Cas systems direct RNA-guided DNA integration. Nature. 571, 219-225 (2019).
64. M. Krupovic, K. S. Makarova, P. Forterre, D. Prangishvili, E. V. Koonin, Casposons: a new superfamily of self-synthesizing DNA transposons at the origin of prokaryotic CRISPR-Cas immunity. BMC Biol. 12, 1-12 (2014).
65. S. M. Crotty, B. Q. Minh, N. G. Bean, B. R. Holland, J. Tuke, L. S. Jermiin, A. V. Haeseler, GHOST: Recovering Historical Signal from Heterotachously Evolved Sequence Alignments. Syst. Biol. 69, 249-264 (2020).

### Example 2

FIG. 62 shows reclustering CRISPR-associated IscB polypeptide at 60% sequence identity reveals novel IscB proteins. The IscB proteins comprise an X domain and a Y domain. CRISPR-associated IscB proteins from 00644 cluster were functional with an NAC PAM (FIG. 63A showing IscB protein of locus JGI accession Gaa0099850_1002913; FIG. 63B showing IscB protein of JGI accession Ga0348337_018242; FIG. 63C showing IscB protein of locus JGI accession Ga0208542 1002724).

### Materials and Methods

### Profile Curation

Initial IsrB sequences were curated using NCBI's PSI-BLAST on the NR database with 8 iterations from a starting seed sequence up to a maximum of 20000 target sequences (Camacho, C. et al. (2009), BMC Bioinformatics. 10, 421). Strong filtering parameters (expect threshold of 1e-5 and PSI-BLAST threshold of 1e-6) were selected to reduce the accumulation of unrelated proteins also containing HNH domains, such as restriction enzymes or homing endonucleases. All proteins smaller than 260 aa were discarded. The remaining proteins were then aligned using MAFFT FFT-NS-1 and partial proteins as well as proteins with poor alignment coverage to the HNH domain were discarded (Kotah, K., et al. (2013), Mol. Biol. Evol. 30, 772-780). The filtered set was then clustered using MMSeqs2 at 70% sequence identity with a minimum coverage of 70%. The MMSeqs2 representatives for each cluster were then aligned using MAFFT-einsi (Steinegger, M. et al. (2017), Nat. Biotechnol. 35, 1026-1028). The resulting alignment was further split into multiple domains (PLMP, RuvC-I, RuvC-II, HNH, and RuvC-III) in order to create distinct HHAlign profiles for the respective regions (Steinegger, M. et al. (2019), BMC Bioinformatics. 20, 473). For HMMER profiles, the PLMP, HNH, and RuvC-III were used to create separate profiles. RuvC-I, BH, and RuvC-II were combined into a single HMMER profile to reduce false positives (Eddy, S., et al. (2018), Proc. Natl. Acad. Sci. U.S.A 115, E5307-E5316).

Due to the extensive diversity in TnpA, a PSI-BLAST search resulted in more than 20000 homologous sequences. Because TnpA consists of a single contiguous catalytic domain, HHblits was instead used to identify more non-redundant homologs using an E-value cutoff of 1e-3, minimum hit probability of 80% and 8 iterations on the UniRef30_2020_06 database (Steinegger, M. et al. (2019), BMC Bioinformatics. 20, 473). The resulting proteins were aligned using MAFFT-einsi, and partial proteins were removed (Kotah, K., et al. (2013), Mol. Biol. Evol. 30, 772-780). The resulting alignment was used to create an HMMER profile.

### Identification of IseB, IsrB, and IshA

All prokaryotic (bacterial+ archaeal) genomes were downloaded from NCBI, and NCBI WGS, as well as JGI projects for which express permission was given, and were combined into a single prokaryotic database. ORFs on all contigs were predicted with TTG, TAG, and TAA stop codons, and ATG start codons (with a minimum size of 55 aa), allowing for alternative start codons GTG (if it produced a minimum ORF size of 100 aa), TTG (if it produced a minimum ORF size of 100 aa) or CTG (if it produced a minimum ORF size of 300 aa). In the case of multiple potential start sites with different start codons for the same potential ORF, all potential start sites were listed in order of increasing corresponding ORF size. A best start site was iteratively selected by traversing the list and selecting a new best start site based on the previous one with the first item in the list selected as the initial best. GTG/TTG start sites were accepted as best if they would make the ORF 20 aa longer than the current best start site, while CTG was accepted only if it would make the protein 40 aa longer than the current best start site. Any ATG start site larger than the current best was automatically selected. ORFs sharing the same stop location and strand (+/−) as an existing protein annotation were discarded in favor of the existing annotation. All ORFs were then searched using HMMER and the 4 IscB profiles with a minimum bit score of 18. Any ORF/protein with a hit to any of the 4 domains was considered a protein of interest (POI) and retained for further analysis. For redundancy reduction, all proteins were then clustered using MMSeqs2 at 90% sequence identity with 85% coverage. Within each 90% cluster, proteins smaller than the 80th percentile in length were discarded. Of the remaining proteins, proteins containing X (ambiguous) amino acids were discarded unless their removal would result in an empty set of sequences. Of the remaining proteins, the sequence with the largest minimum distance from either the start or the end of the protein-coding sequence to the edge of the respective contig, that is, the protein sequence farthest away from either edge of the containing contig, was selected as the cluster representative. Representative proteins with start or end sites within 200 bp of contig edges were considered partial and were subsequently discarded. These remaining representative sequences constituted non-redundant POIs. The non-redundant POIs were then further clustered into “clusters” (as opposed to non-redundant sequences) using MMSeqs2 at 65% sequence identity and 70% sequence coverage. For each cluster, the representative sequence was taken to be the sequence with the 90th percentile length in the cluster. The use of 90th percentile reflect the skew towards protein fragments that are often observed in large clusters. Unless specified otherwise, when referring to a cluster's components, such as the protein ORF (e.g. IscB cluster 2089) or associated RNA sequence (e.g. 2089 ncRNA), it is the respective component of the representative locus for that cluster that is being referenced.

### IscB-IsrB-Cas9 Ruv (BH and RuvC BH HNH Domain Based Phylogenetic Analysis

Cas9s identified from the IsrB domain-based search exhibited monophyletic branching from a RuvC-based tree, however not all Cas9s were identified from this search. To expand the space of Cas9s included in the analysis, Cas9 profiles from the Koonin Lab (30), TIGRFAM, and profiles made from MAFFT alignments of Cas9 proteins from CRISPRDisco were used to identify additional Cas9 proteins not found in the initial IsrB domain-based search using HMMER with a minimum bit score of 25 and protein length of 500 aa (Kotah, K. et al. (2013), Mol. Biol. Evol. 30, 772-780; Crawley, A. et al. (2018), CRISPR J. 1, 171-181; Haft, D. et al. (2001), Nucleic Acids Res. 29, 41-43). Cas9 proteins from the IsrB domain search were combined with the Cas9 proteins from the Cas9 search and deduplicated. The Cas9 ORF start sites were then refined using GLIMMER (Delcher, A, et al. (1999), Nucleic Acids Res. 29, 41-43). The superset of Cas9 proteins were then clustered 90% sequence identity with 85% coverage and redundancy reduced in the same manner as for the IsrB search. The superset of non-redundant Cas9 proteins were then reclustered at 50% sequence identity and 60% coverage. The choice of 50% sequence identity reflects the difference in the size of conserved regions between IsrBs and Cas9s with approximately 100 aa more slowly evolving in the key regions (RuvC, BH, and HNH). The clustering criteria of 65% minimum sequence identity for 400 aa proteins is functionally similar to 65%-100/400+100/1000=50% sequence identity for larger 1000 aa Cas9s with the same conserved domains spanning approximately 100 aa. For each cluster, the representative sequence was taken to be the sequence with the 90th percentile length in the cluster.

Clusters were additionally filtered for homology to IsrB/Cas9 catalytic domains using profile-profile comparison. Specifically, for each cluster, the non-redundant sequences in the cluster were aligned using MAFFT in order to generate hhm inputs for HHAlign. These resulting alignments were then compared to the 6 IscB domain alignments using HHAlign (for IscB/IsrB clusters), or the corresponding 8 Cas9 domain hhm profiles of the equivalent regions (RuvC-I, Bridge Helix, REC1, REC2, RuvC-II, HNH, RuvC-III). IscB/IsrB clusters were selected for further analysis if at least two hits to any of the IscB RuvC-I, RuvC-II, or RuvC-III domains were identified through HHAlign (with a minimum bit score of 17). Cas9 clusters were selected for further analysis if at least two hits to any of the IscB RuvC-I, RuvC-II, or RuvC-III domains were identified through HHAlign (with a minimum bit score of 17), or if any two hits to the Cas9 RuvC-I, RuvC-II, RuvC-III domains were identified through HHAlign (with a minimum bit score of 17). The passing Cas9 clusters were then pooled with the passing IscB clusters for phylogenetic analysis.

IscB, IsrB and Cas9 obtained from the above filtering criteria were aligned using MAFFT-x2 (two iterations) and BLOSUM62 scoring (default if not otherwise specified). The RuvC-I and BH regions of Cas9 did not align with the RuvC-I and BH regions of IscB and IsrB due to the different domain architectures and sizes of these divergent protein families. RuvC-I and BH were manually grouped and realigned using MAFFT-x2. Sequences with poor coverage at the RuvC-I domain region of the alignment were removed if their HHAlign score to either the IscB-RuvC-I or Cas9-RuvC-I profiles were less than 21. Small proteins with both Cas9-like and IscB-like sequences containing HHAlign hits to RuvC-I and BH typically did not have correct alignments with RuvC-I and BH relative to all other proteins due to their hybrid nature. For such proteins, all amino acids between the N-terminus and RuvC-II were grouped together as RuvC-I and BH for alignment. RuvC-I and BH regions were then realigned using MAFFT-linsi. IsrB aligned columns from the HNH domain were moved to the RuvC-II column group. RuvC-II, RuvC-III, HNH, and PLMP domains were then sequentially aligned using MAFFT-linsi. Excess regions between the BH domain and RuvC-II were then aligned using MAFFT-einsi and BLOSUM30 for identification of REC-like insertions. Sequences with no or poor alignment to any of the RuvC or BH domains were removed. The resulting alignment was then used for phylogenetic analysis.

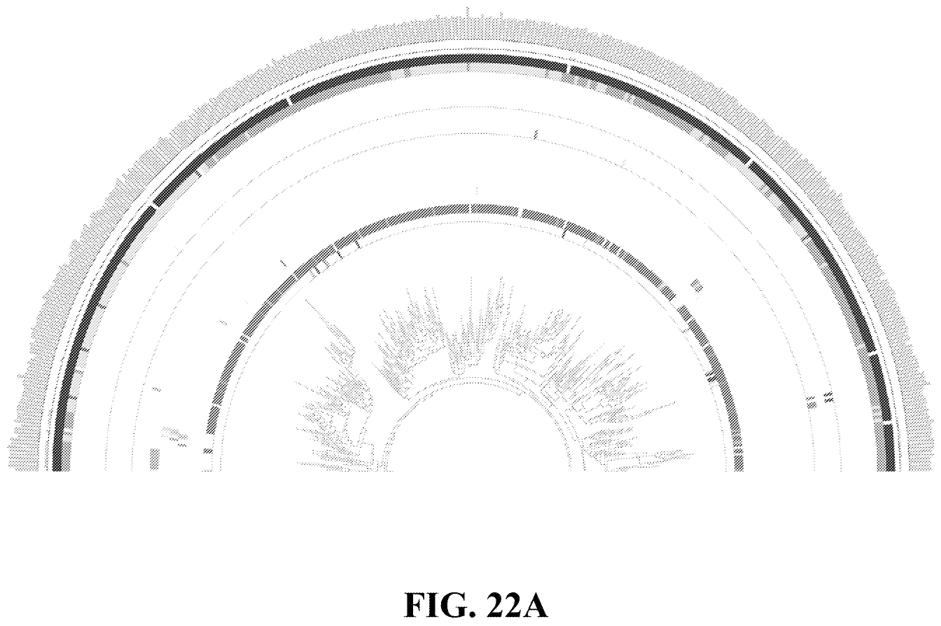

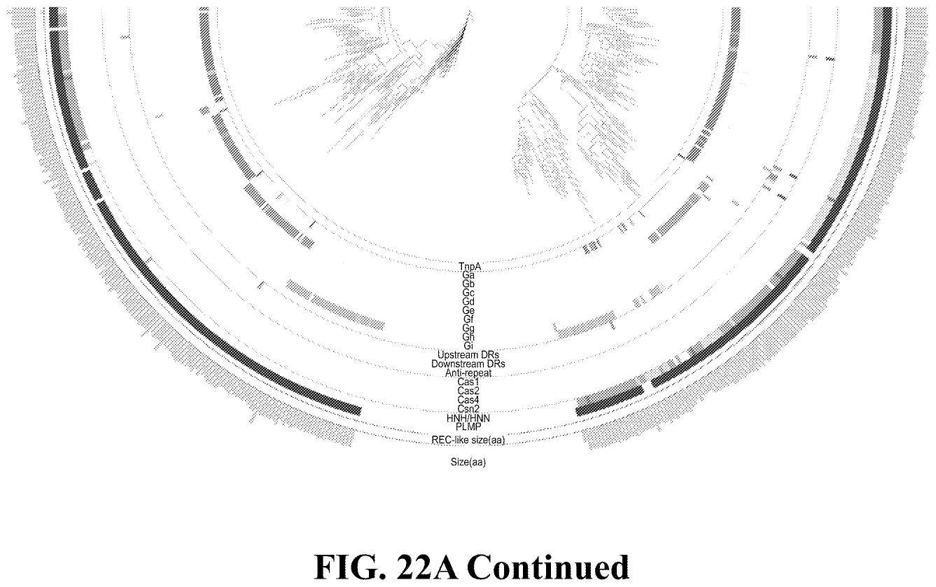

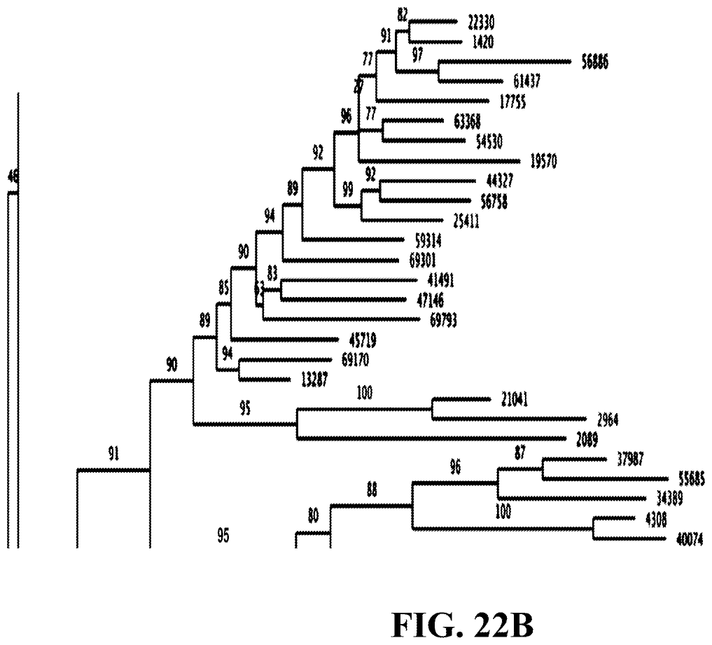

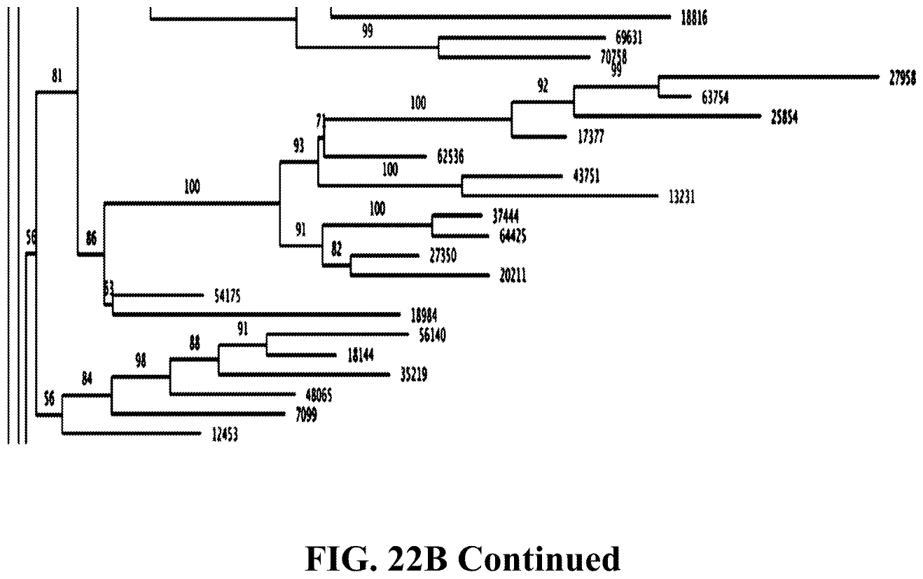

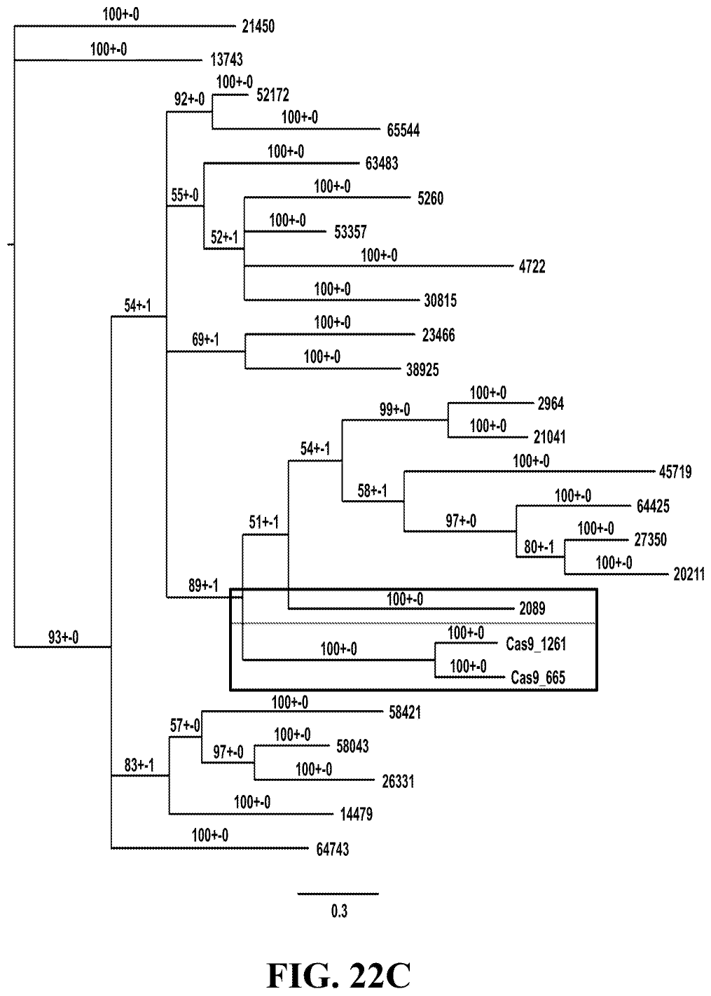

All domains not common to all 3 types of proteins, namely PLMP, REC1, REC2, PI domains, and IscB/IsrB C-terminal domains were removed from the alignment, leaving a trimmed alignment containing only the highly conserved portions of the RuvC-I, BH, RuvC-II, and RuvC-III domains, creating a RuvC/BH alignment containing IscB, IsrB, and Cas9. Another alignment was created containing only RuvC-I, BH, RuvC-II, HNH, and RuvC-III domains for only IscB and Cas9, termed the RuvC/BH/HNH alignment. For both of these alignments, clusters with dead representative sequences (sequences with key catalytic sites mutated) were removed. Specifically, the positions filtered were the RuvC-I conserved D, RuvC-II conserved E, HNH Conserved H (when applicable), and RuvC-III conserved D and H. Positions (columns) with more than 75% gaps were also removed. Symmetry tests implemented in IQ-Tree 2 were used to identify potential phylogenetic violations for the alignments (FIG. 22) (Naser-Khdour, S. et al. (2019), Genome Biol. Evol. 11, 3341-3352). The RuvC/BH/HNH alignment displayed significant violation to the stationarity assumption of the three main assumptions used in typical phylogenetic analysis (reversibility, stationarity, homogeneity). As the alignment contained too many taxa for the use of heterotachy models in IQ-Tree 2, Applicants use a subtractive approach to identify the source of the stationarity violation. Preliminary analyses showed that the major clade of II-B Cas9s consistently split from the rest of Cas9s with a branch length of ≥1, suggesting its accurate placement along the tree might be difficult. Applicants removed the major clade of II-B Cas9s from the RuvC/BH/HNH alignment, which substantially reduced the stationarity violation as determined by marginal symmetry test p-value (FIG. 22). Applicants also created another alignment only consisting of IscB and Cas9s from the early stages of Cas9 evolution, which completely eliminated any stationarity violation. For both of these additional alignments, catalytically inactive variants were removed as described for the RuvC/BH and RuvC/BH/HNH alignments, and positions (columns) with more than 75% gaps were removed. For each alignment, substitution model selection was performed using the ModelFinder tool implemented in IQ-Tree 2 (Kalyaanamoorthy, S. et al. (2017), Nat. Methods. 14, 587-589). Optimal models were selected using the Akaike Information Criterion corrected for small sample sizes (AICc). In most cases, the AICc best model differed from the Bayesian Information Criterion (BIC) or AIC (standard Akaike Information Criterion), and some analyses were run for both sets of models; however, AICc was generally preferred due the small sample size correction. Then, for each alignment, phylogenetic trees were built using multiple methods for cross comparison (IQ-Tree 2, RAXML, MrBayes). While FastTree2 was used for quick visualizations of phylogenetic information, the likelihood scores obtained using this method were substantially worse than those for IQ-Tree 2, RAXML, or MrBayes (Mihn, B., et al. (2020), Mol. Biol. Evol. 37, 1530-1534; Price, M., et al. (2010), PLOS One. 5, e9490; Altekar, G. et al. (2004), Bioinformatics. 20, 407-415; Stamatakis, A. et al. (2014), Bioinformatics. 30, 1312-1313). As a result, FastTree2 was not used for comprehensive cross comparisons.

For FIG. 9A, a hybrid tree approach was used in order to condense the information pertinent to Cas9 while maintaining phylogenetic accuracy. For this approach, a subsample of Cas9 clusters were selected from the alignment, in addition to the complete set of IscB and IsrBs. The resulting sub-alignment was then used for phylogenetic inference using IQ-Tree 2 with identical parameters. Due to the potentially skewed Cas9 related information present in the sub-alignment, placement of the Cas9 lineage was inferred from the tree built with the original alignment. This was done by detaching the Cas9 branch from the sub alignment tree and replacing the original Cas9 branch on the original tree with the smaller detached branch. The branch selected for grafting was the branching between Cas9_849 and all other Cas9s, as this region shared the same topology between both trees. The ordering of Cas9 subtype evolution was checked for consistency between each tree to ensure compatibility after having substantially downsampled Cas9 proteins.

### IscB/IsrB ωRNA Discovery, Curation, and Analysis

Using results from just the IscB domain search, all representatives from clusters with at least 3 proteins and at least 2 HHAlign hits to any of RuvC-I, RuvC-II, or RuvC-III with at least 17 bit score were collected. All regions upstream (−300 bp to +200 bp of the start codon), and downstream (−200 bp to +300 bp of the stop codon) for all IscB and IsrB proteins were aligned separately using MAFFT-einsi. The upstream alignment demonstrated large conserved regions outside of the typical CDS boundaries of the IscB/IsrB. The downstream alignment did not contain any large conserved region and was discarded for further analysis. Individual sequences in the conserved upstream region were folded using ViennaRNA RNAFold (Lorenz, R. et al. (2011), Algorithms Mol. Biol. 6, 1-14). Sequences in the alignment were split into separate groups on the basis of conservation to key distinct regions in the alignment. The main group was labeled G1a and spanned a large number of IscB ωRNAs. R-scape was used to infer covariance-folded RNA structures that correct for phylogenetic correlations (Rivas, E. et al. (2017), Nat. Methods. 14, 45-48). The R-scape parameters used for all profiles was an E-value threshold of 1e-2 and a gap threshold of 0.75. CMbuild from Infernal was used to optimize the RNA alignments and build covariance models (CMs) (Nawrocki, E. et al. (2013), Bioinformatics. 29, 2933-2935). R2R was used to visualize the resulting RNA structures (Weinberg, Z. et al. (2011), BMC Bioinformatics. 12, 3). Additional alignment groups for IscB/IsrB ωRNAs (G1b-i) were iteratively created based on conserved upstream regions (relative to the ORF) from clades of IscB/IsrB in the RuvC tree that were not strongly associated with existing ωRNA groups. Models for these groups were built in the same manner, except the consensus secondary structure identified using ViennaRNA was used in place of the R-scape structure when the sample size was too small to permit an accurate covariance-folded structure. Due to this small sample size for the CRISPR-associated IscB ncRNA, nucleotides with ≥97% identity and nucleotides with ≥90% identity are the same by definition and were displayed as being ≥90% to avoid overstating their level of conservation in the presence of limited data. Pseudoknots were not explicitly encoded in the covariance models for Infernal. The structure for the hybrid CRISPR/ωRNA associated with CRISPR-associated IscBs was inferred in the same way, using the consensus secondary structure in lieu of the covariance-folded structure due to small sample size.

All IscB/IsrB ωRNA CM profiles were then searched against the prokaryotic database using cmsearch with a minimum bit score of 20 to identify potential ωRNAs (Nawrocki, E. et al. (2013), Bioinformatics. 29, 2933-2935).

Overlapping profile hits were resolved by assigning the group of overlapping hits to the profile hit that produced the highest bit score. ωRNAs were annotated using the natural target search analysis

### Natural Target Search

For the target search, a separate database was curated consisting of plasmids and bacteriophage/archaeal viruses from the NCBI microbial genomes portal. The 30 bp upstream from each ωRNA scaffold as determined by CMsearch along with the generated profiles was used as the corresponding guide sequence for that ωRNA. Guides were then searched against the prokaryotic database or the plasmid/viral database for potential spacer matches using a minimum bit score cutoff of 44. To remove candidate target hits that are related to the original guide/ωRNA pair, a separate pruning step was performed. For each target hit, the corresponding ωRNA scaffold of the guide that generated the hit was separately blasted against a 300 bp window around the target. For this second search, if the ωRNA scaffold had a match within the window with a bit score of 44 or higher and was found within 15 bp of the identified target, the target was discarded for being too similar to the original locus.

Target hits were then searched for evidence of transposition. For each guide/target pair, the sequence consisting of up to 2000 bp downstream from the end of the ωRNA scaffold was used as a 3′ prime seed. This seed region was then searched against the target contig using blastn with a minimum bit score of 44. Hits from this search were discarded if the strand was not the same as the guide/target match strand. Moreover, hits that were not both ≤50 bp downstream from the guide/target match site and ≤15 bp upstream from the guide/target match site were discarded for being implausible insertion sites. If any 3′ seed hits remained at this stage, the guide/target pair was considered to be associated with transposon insertion/transposition. In this case, of the remaining 3′ seed hits, the most upstream hit was selected, which along with the guide/target site demarcated the 3′ insertion site flanking region and 5′ insertion site flanking region respectively. Guide/target pairs that did not have an identifiable 3′ insertion site flanking region were not considered insertion linked and were discarded unless their guide/target match bit score was ≥50, which approximately corresponded to an E-value of 0.02 for the given size of the prokaryotic database.

To determine if prophage sequences might be a target for ωRNAs, all guide/target pairs were analyzed to detect if the guide targeted ORFs. Specifically, a guide was considered ORF targeting if any corresponding target was found to overlap with an ORF in the given database. All targeted ORFs were then searched for possible PFAM hits using HMMER and the Pfam34 database (Mistry, S. et al. (2021), Nucleic Acids Res. 49, D412-D419) and a minimum bit score of 18. All ORFs that had any hit to any phage Pfam profiles listed in Table 12 were considered phage targets, and the corresponding guide was labeled as phage ORF targeting.

ωRNAs were then further annotated as being near IscB/IsrB fragments based on the presence of any nearby ORF (≤2000 bp) with an HMMER hit of 18 bits or higher to any of the 4 IscB HMM profiles. ωRNAs were then clustered as follows. ωRNA scaffold sequences were clustered at 95% sequence identity using cd-hit-est using only global+/+ strand comparisons.

Similarly, all spacers were clustered at 95% sequence identity using cd-hit-est using only global+/+ strand comparisons. ωRNAs were then grouped according to both their RNA scaffold cluster id (ωRNA_id) and their spacer cluster id (spacer_id). Each (ωRNA_id, spacer_id) group was then classified as being associated near IscB if any ωRNA in the group was considered near IscB/IsrB fragments. Then, for each ωRNA_id, the fraction of all (ωRNA_id, spacer_id) pairs considered associated with IscB/IsrBs was computed. If this fraction was ≤0.75, the ωRNA_id was classified as being associated with standalone ωRNAs, and this classification was propagated back to all IscB/IsrB ωRNAs according to their ωRNA_id.

### Anti-Repeat Determination

For CRISPR associated IscB, IsrBs, and Cas9s, anti-repeats corresponding to likely tracrRNA regions were identified as follows. For each locus, the closest predicted CRISPR array to the locus within 10 kbp of the protein of interest (POI) (namely IscB, IsrB, or Cas9) was selected. If no such array was identified, the analysis was not performed for the given locus. Next, edit distance between each direct repeat in the selected CRISPR array was computed. The resulting edit distance matrix was used to a medoid direct repeat that had the lowest average edit distance to all other direct repeats in the CRISPR array. The medoid direct repeat was then searched against the 10 kbp window around the POI using blastn with a word size of 4. Hits with bit score <20 were discarded. Hits that mapped to any predicted CRISPR array were used for calibration. Specifically, all such CRISPR array matching hits were collected into a list, and the 20^{th} quantile bit score of such hits was denoted the min_crispr_score. Any hit that was found ≤40 bp of any CRISPR array was discarded as a potential anti-repeat due to its high proximity to CRISPR arrays suggesting the hit could simply be another CRISPR direct repeat. Hits occurring inside the protein of interest were also discarded. Remaining hits occurring ≤500 bp from either the POI or any predicted CRISPR array were retained. For each hit in this final list, if the region of the query median direct repeat that produced the hit does not reach ≤3 bp of either edge of the direct repeat, the hit was discarded as a potential anti-repeat due to the likely inability to produce a stable lower stem region for the putative repeat-anti-repeat stem-loop. If any hits remained, these hits were considered anti-repeats for the POI.

Anti-repeats were then used to predict potential tracrRNAs for specific Cas9s. This process involved taking the anti-repeat region and extending it until the boundary of the neighboring feature (either protein gene or CRISPR array). TracrRNAs were predicted in this manner for the tracrRNAs shown in FIG. 9B.

### Cluster Annotation

All 10 kb genomic frames (10 kb region around the protein-coding gene of interest (POI)) were collected, and encompassed all unique/non-redundant IscB, IsrB, and Cas9 genes (according to their 90% redundancy reducing clustering id, denoted mc_id). CRISPR arrays were identified using CRT (Bland, C. et al. (2007) BMC Bioinformatics. 8, 209). TnpA was predicted for all ORFs within 10 kb of a POI using HMMER with a minimum bit-score cutoff of 18.0. All ωRNA annotations from the natural target search were merged with the corresponding genomic frames when applicable. RNA profile hits with a bit-score below 35 were discarded. For each genomic frame, all ωRNAs predictions were merged with the POIs according to their genomic locations. All ωRNAs further than 250 bp away from either end of the POI were considered to be unrelated to the POI. Any ωRNAs closer than 250 bp were considered related to the POI, and in the case of multiple such ωRNAs, only the closest one to the POI was considered for tabulation purposes. For each POI, a locus boundary was then computed as follows. A minimal locus interval [L, R] was set to encompass the POI and associated ωRNA when applicable. Then, iteratively, all other ORFs and CRISPR arrays in the contig were considered for addition into the locus interval, upon which a new minimal interval [L′, R′] would be computed to encompass all elements in the locus. Specifically, an ORF was added to the locus if it was ≤1000 bp from the existing interval and contained a Cas1, Cas2, Cas4, or Csn2 HMM profile hit with a minimum bit score of 25 as determined by hmmsearch. CRISPR arrays were included if they were ≤500 bp from the existing interval and contained ≥3 direct repeats and had a median direct repeat length of ≥25 bp. The locus determination process was terminated once no new elements were being added, resulting in a final locus interval [L, R], which Applicants termed the locus bounds. ORFs with HMMER hits to TnpA with a minimum bit score of 14 was then considered mhpA and a part of the locus if they were ≤500 bp away from the locus bounds ([L, R]).

For tabulation of cluster annotations, all POIs and corresponding locus information were grouped by cluster id (c_id). POIs with ‘X’ amino acids in the sequence translation were discarded. Moreover, POIs with ≤250 bp distance to either edge of the contig were also discarded. The resulting locus features (features within the locus bounds [L, R])), and tnpA association were then used to tabulate aggregate metrics, such as average CRISPR association (fraction of loci in the cluster that have a CRISPR array within the locus bounds), tnpA association, and various cas gene associations. Anti-repeat association rates were also calculated in this way using anti-repeats as determined above, where loci that did not contain a CRISPR directly inside the locus bounds ([L, R]) were considered to not have an anti-repeat. For natural target search tabulations, the associated ωRNA in the locus bounds for POI closest to the POI was used. The fraction of associated ωRNAs in the cluster targeting genomic regions, plasmids, and phage were tabulated. Similarly, the fraction of associated ωRNAs associated with standalone ωRNAs for the cluster was also calculated. Lastly, the fraction of associated ωRNAs in the cluster that demonstrated evidence of insertion/transposition was also computed for each cluster.

For tabulation of protein domains, the hhm profile for the alignment of non-redundant sequences from the cluster was used to identify domain hits. Specifically, the cluster was considered to have the PLMP domain if the cluster alignment had an HHAlign match to the IscB PLMP domain with a bit score ≥30. Likewise, the cluster was considered to have the HNH domain if the cluster alignment had an HHAlign match to the IscB HNH domain with a bit score ≥25. As the HNH has multiple variants of the motif (often being replaced by HNN as in most Cas9s), the identity of the last amino acid in the HNH motif (e.g. HNH vs HNN) was computed for the cluster according to the corresponding position in alignment of representative sequences used for the phylogenetic analysis.

The protein length for each cluster was taken to be the non-gapped length of the cluster representative in the alignment containing all IscB, IsrB, and Cas9 cluster representative sequences (IscB_IsrB_Cas9_full_gappy.fasta). The calculated protein length therefore reflected the trimming of large (>50 aa) non-homologous sequences at the protein termini as determined from the IscB_IsrB_Cas9_full_gappy.fasta file. REC-like regions were taken to be the region directly between the Bridge Helix and the RuvC-II domain as found in the alignment of cluster representatives. The REC-like regions are found in IscB_IsrB_Cas9_REC_only.fasta. Lengths of REC-like insertions for each cluster were taken to be the non-gapped lengths from the REC alignment. GraPhlAn was used to visualize the resulting information on different phylogenetic trees (Asnicar, F. et al. (2015), Peer J. 3, e1029).

Throughout the text, where fraction of loci containing a specific feature are computed, the analysis considered only the non-inactivated IsrB loci, as determined by RuvC/BH alignment, and the non-activated IscB and Cas9 loci as determined by the RuvC/BH/HNH. In addition, proteins from cluster 34507 were also included despite being considered an inactivated cluster because the cluster contains active CRISPR-associated IscB variants.

### IscB/IsrB ωRNA Phylogenetic Analysis

All iscB/isrB associated ωRNAs from the loci of cluster representatives were collected.

For CRISPR-associated ωRNAs, the entire region spanning the CRISPR array and the remainder of the ωRNA scaffold was taken to be the corresponding RNA for the system. These RNAs were then converted to DNA sequences and aligned using MAFFT-ginsi to identify conserved regions spanning multiple types of RNAs. A phylogenetic tree was then constructed using IQ-Tree 2 with 5000 rapid bootstraps with hill climbing, a GTR substitution model with 4 Gamma rate categories.

To analyze the relationship between II-D tracrRNAs and ωRNAs, cmsearch was used to identify if the predicted II-D tracrRNAs contained any homology to IscB/IsrB ωRNAs using the corresponding IscB/IsrB ωRNA profiles. Based on the potential homology determined this way, a small panel of ωRNAs that were highly related to the II-D tracrRNAs were combined with synthetic single guide sequences from II-D systems constructed by concatenating the DR with ‘AAAA’ and the predicted tracrRNA. These combined sequences were then aligned using MAFFT. The resulting alignment was used for Bayesian phylogenetic analysis with MrBayes with 2 chains at a delta temperature of 0.025 with 8 independent runs for 5M generations. A standard GTR model with gamma rates and 4 categories was used.

### Binning of Yellowstone Lake Metagenomes

Tetranucleotide frequencies of assembled metagenomes (contigs >5 kb) provided an input for the t-stochastic neighbor embedding (t-SNE) analysis, which reduced the data to two dimensions (Maaten, L. et al. (2008), Journal of Machine Learning, 9, 2579-2605). Applicants used openTSNE (Poličar, P. et al. (2019, bioRxiv (2019), doi: 10.1101/671404) because of its speed and scalability. The perplexity parameter was set at 40, while the learning rate and the number of iterations were variable. Typically, both were set to larger values for bigger datasets. A grid search coupled with silhouette scores (Rousseeuw, P. et al. (1987), J. Comput. Appl. Math. 20, 53-65) was used to find optimal parameters for density-based clustering (Campello, R. et al. (2013), Advances in Knowledge Discovery and Data Mining, Lecture Notes in Computer Science, pp. 160-172) (HDBSCAN) of data points from the t-SNE plot (MD and WPI, in preparation). DNA sequences were grouped into metagenome-assembled genomes (MAGs), and their completeness was evaluated using CheckM (Parks, D. et al. (2015), Genome Res. 25, 1043-1055). The GTDB Toolkit (Chaumeil, P. et al. (2019), Bioinformatics doi: 10.1093/bioinformatics/btz848) was used to classify MAGs into taxonomic groups based on the Genome Taxonomy Database.

### Identification of Guide Encoding Mechanisms for IscB_IsrB ωRNA

Upon complete classification and curation of all major ωRNA types according to IscB and IsrB, the genome of K. racemifer was searched for all instances of IscB and IsrB using HMMER with the previously described profiles. The genome was also searched for all instances of IscB/IsrB ωRNAs using CMsearch with the G1a-G1i RNA covariance models. Occurrences of multiple nearly identical IscBs associated with nearly identical ωRNAs were identified with BLASTn and was classified as transposon expansion. Occurrences of ωRNAs without detectable IscBs or IsrBs within 500 bp on the same strand were classified as standalone trans-acting ωRNAs. In some instances, ωRNAs and corresponding IscB/IsrBs were separated by the insertion of unrelated transposons between them. In such cases, the ωRNA was not considered a trans-acting ωRNA.

All covariance models were searched against our prokaryotic genome database. Examples with multiple ωRNAs on the same strand within 300 bp were retained for further analysis and categorized as ωRNA arrays.

### Identification of Eukaryotic IsrB Orthologs.

All eukaryotic genomes were downloaded from NCBI. In order to capture all possible IscBs, existing gene models were discarded for this analysis. All DNA sequences were translated into 6 frame amino acids translations and split into ORFs by splitting across stop codons (*).

Each ORF was then searched for IscB domains using the HMMER profiles generated by the IscB profile curation step. ORFs with hits to both IscB HNH and RuvC domains were retained for further analysis. The regions around the ORFs were then searched for IscB-linked ωRNAs using CMsearch and the IscB-linked ωRNA covariance models created in this study.

### Taxonomy Analysis of IscB, IsrB, and Cas9

Taxonomy information for available genomes were obtained from the NCBI microbial genomes portal. Genomes which lacked taxonomic information or metagenomes were discarded for this analysis. Genes were classified into IsrB, IscB, Cas9, or other on the basis of their cluster id. The fraction of genomes in each taxonomic group containing IsrB, IscB, or Cas9 was computed. For each gene type (IscB, IsrB, and Cas9) the distribution of the number of genes per genome in each taxonomic group was calculated. Box plots of gene counts per taxonomic group was then computed using Python.

### TnpB Curation and Analysis of ωRNA

Examples of IscB-linked ωRNA from K. racemifer were searched in the K. racemifer genome using BLASTn. Hits in the vicinity of IscB or IsrB were discarded. Multiple partial hits were found in the vicinity of TnpB, with the hit being always downstream of the TnpB gene.

Exploration of these hits showed that multiple TnpBs shared transposon ends with IscB. An upstream and downstream locus conservation analysis was performed for related TnpB loci as done for IscB. As TnpB is highly diverse, only TnpBs that were identifiable via high similarity in an mmseqs2 search were included. The RNA-seq traces were then used to identify the 5′ RNA boundary on the TnpB ωRNAs in the conservation analysis. These ωRNAs from the conservation analysis were then extracted and used to create an R-scape secondary structure with an E-value threshold of 1e-2 and a gap threshold of 0.75.

### Expression and Purification of CRISPR-Associated IscB RNPs

To purify the CRISPR-associated IscB in complex with the ncRNA of its native locus, the human codon optimized CRISPR-associated IscB protein was cloned into a modified pET45b (+) backbone with an N-terminal His14-MBP-bdSUMO tag (Frey, S. et al. (2014), J. Chromatogr. A. 1337, 95-105), and C-terminal twin-strep tag. The non-coding part of the CRISPR-associated IscB locus was cloned into a separate pCOLADuet-1 vector, and co-expressed with the CRISPR-associated IscB protein in Rosetta (DE3) pLysS strain (EMD Millipore) (Tables 5, 9). Cells were grown at 37° C. in the terrific broth (TB) medium supplemented with 100 μg/ml ampicillin, 25 μg/ml kanamycin, and 34 μg/ml chloramphenicol until reaching an optical density (OD600) of 0.3, then shifted to 18° C., and grown further until OD600 was 0.7. The bacterial culture was induced overnight at 18° C. with 0.2 mM isopropyl β-D-1thiogalactopyranoside (IPTG) and harvested by centrifugation. The cell paste was resuspended in lysis buffer (50 mM Tris pH 8, 200 mM NaCl, 5% glycerol, 5 mM MgCl₂, and 1 mM DTT) supplemented with protease inhibitors (PMSF and Roche complete, EDTA-free), and lysed by two passes with a high-pressure homogenizer (LM20 Microfluidizer, Microfluidics). The lysate was cleared by centrifugation, and the soluble fraction was gently mixed with Strep-Tactin Superflow Plus resin (Qiagen) at 4° C. The resin was first washed with lysis buffer, then briefly with buffer A (50 mM Tris pH 8, 1 M NaCl, 5% glycerol, 5 mM MgCl₂, and 1 mM DTT), and buffer B (50 mM Tris pH 8, 500 mM NaCl, 5% glycerol, 5 mM MgCl₂, and 1 mM DTT) on a gravity flow column. Bound RNP was then eluted in buffer B supplemented with 5 mM desthiobiotin (Sigma), and the N-terminal solubility tag was cleaved using bdSENP1 protease.

Once the boundaries of the co-purified RNA were defined by small RNA sequencing, the predicted ncRNA sequence was cloned downstream of a T7 promoter in a pCOLADuet-1 vector for inducible expression. The CRISPR-associated IscB-ncRNA complexes used for in vitro cleavage assays were then prepared following the same procedure explained above.

Small RNA Sequencing: Heterologous Expression in E. coli

Stb13 chemically competent E. coli were transformed with plasmids containing the locus of interest. A single colony was used to seed a 5 mL overnight culture. Following overnight growth, cultures were spun down, resuspended in 750 μL TRI reagent (Zymo) and incubated for 5 min at room temperature. 0.5 mm zirconia/silica beads (BioSpec Products) were added and the culture was vortexed for approximately 1 minute to mechanically lyse cells. 200 μL chloroform (Sigma Aldrich) was then added, culture was inverted gently to mix and incubated at room temperature for 3 min, followed by spinning at 12000×g at 4° C. for 15 min. The aqueous phase was used as input for RNA extraction using a Direct-zol RNA miniprep plus kit (Zymo). Extracted RNA was treated with 10 units of DNase I (NEB) for 30 min at 37° C. to remove residual DNA and purified again with an RNA Clean & Concentrator-25 kit (Zymo). Ribosomal RNA was removed using a RiboMinus Transcriptome Isolation Kit for bacteria (Thermo Fisher Scientific) as per the manufacturer's protocol using half-volume reactions. The purified sample was then treated with 20 units of T4 polynucleotide kinase (NEB) for 6 h at 37° C. and purified again with an RNA Clean & Concentrator-25 (Zymo) kit. The purified RNA was treated with 20 units of 5′ RNA polyphosphatase (Lucigen) for 30 min at 37° C. and purified again using an RNA Clean & Concentrator-5 kit (Zymo). Purified RNA was used as input to an NEBNext Small RNA Library Prep for Illumina (NEB) as per the manufacturer's protocol with an extension time of 60 s and 16 cycles in the final PCR. Amplified libraries were gel extracted, quantified by qPCR using a KAPA Library Quantification Kit for Illumina (Roche) on a StepOne Plus machine (Applied Biosystems/Thermo Fisher Scientific) and sequenced on an Illumina NextSeq with Read 1 42 cycles, Read 2 42 cycles and Index 1 6 cycles. Adapters were trimmed using CutAdapt (Martin, M. et al. (2011), EMBnet.journal. 17, 10-12) and mapped to loci of interest using Bowtie2 (Langmead, B. et al. (2012), Nat. Methods. 9, 357-359). Filled reads were obtained and filled reads longer than 200 bp were visualized using a custom Python script.

### Ribonucleoprotein

RNPs were purified as described. 100 μL concentrated RNP was used as input. The above protocol was followed with the following modifications: 300 μL TRI reagent (Zymo) and 60 μL chloroform (Sigma Aldrich) were used for RNA extraction.

K. racemifer

Freeze-dried K. racemifer SOSP1-21 DSM 44963 was obtained from DSMZ (www.dsmz.de/collection/catalogue/details/culture/DSM-44963), resuspended in GYM Streptomyces media (4 g glucose, 10 g malt extract, 4 g yeast extract in 1 L water) and grown in a shaking incubator at 28 C. After 76 days, the culture was spun down, and the above protocol was followed with the following modifications: mechanical lysis using zirconia beads was performed with approximately 30 min of vortexing. Ribosomal RNA was removed using a NEBNext rRNA Depletion Kit (Bacteria) (NEB) as per the manufacturer's protocol and the rRNA-depleted sample was purified using Agencourt RNAClean XP beads (Beckman Coulter) prior to T4 PNK treatment. T4 PNK treatment was performed for 1.5 h and purified with an RNA Clean & Concentrator-5 kit (Zymo). Final PCR in the small RNA library prep contained 15 cycles.

I. tetrasporus

An agar slant of I. tetrasporus UTEX B 2012 was obtained from UTEX, inoculated in Modified Bold 3N Medium (UTEX) and grown at 20° C. in a shaking incubator with 12-hour light/dark cycles. After 18 days, the culture was spun down and the above protocol was followed with the following modifications: ribosomal RNA was removed using the manufacturer's protocol from the RiboMinus Transcriptome Isolation Kit for bacteria (Thermo Fisher Scientific) with probes from the RiboMinus Transcriptome Isolation Kit for bacteria, RiboMinus Plant Kit for RNA-seq and RiboMinus Eukaryote Kit for RNA-seq (all Thermo Fisher Scientific) combined in an equimolar ratio. Eluted RNA from ribosomal removal was concentrated using an RNA Clean & Concentrator-25 kit (Zymo) prior to T4 PNK treatment. Final PCR of the small RNA library prep contained 15 cycles.

### Cloning PAM/TAM Libraries

Target sequences with 8N degenerate flanking sequences were synthesized by IDT and amplified by PCR with NEBNext High Fidelity 2×Master Mix (NEB). Backbone plasmid was digested with restriction enzymes (pACYC: EcoRV; pUC19: Eco88I and HindIII, Thermo Fisher Scientific) and treated with FastAP alkaline phosphatase (Thermo Fisher Scientific). The amplified library fragment was inserted into the backbone plasmid by Gibson assembly at 50° C. for 1 hour using 2×Gibson Assembly Master Mix (NEB) with an 8:1 molar ratio of insert: vector. The Gibson assembly reaction was then isopropanol precipitated by addition of an equal volume of isopropanol (Sigma Aldrich), final concentration of 50 mM NaCl, and 1 μL of GlycoBlue nucleic acid co-precipitant (Thermo Fisher Scientific). After a 15 min incubation at room temperature, the solution was spun down at max speed at 4° C. for 15 min, then the supernatant was pipetted off and the pelleted DNA was resuspended in 12 μL TE and incubated at 50° C. for 10 minutes to dissolve. 2 μL were then transformed by electroporation into Endura Electrocompetent E. coli (Lucigen) as per the manufacturer's instructions, recovered by shaking at 37° C. for 1 h, then plated across 5 22.7 cm×22.7 cm BioAssay plates with the appropriate antibiotic resistance. After 12-16 hours of growth at 37 C, cells were scraped from the plates and midi- or maxi-prepped using a NucleoBond Midi- or Maxi-prep kit (Machery Nagel).

E. coli PAM Screen

100 ng each of a plasmid containing a locus of interest and a target 8N degenerate flanking library plasmid were transformed by electroporation into 30 μL Endura Electrocompetent E. coli (Lucigen) as per the manufacturer's protocol, with 3 biological replicates per locus of interest as well as 3 biological replicates of an empty control. After recovery by shaking at 37° C. for 1 hour, cells were plated across 1 22.7 cm×22.7 cm BioAssay plate with the appropriate antibiotic resistance and grown for 12-16 h at 37° C. Cells were scraped from the plate and mixed well, and 2 mL of the scraped cells were used as input to minipreps (Qiagen). 100 ng miniprepped plasmids were input to PCR to amplify the PAM-containing region with a 12-cycle PCR using NEBNext High Fidelity 2×PCR Master Mix (NEB) with an annealing temperature of 63° C., followed by a second 18-cycle round of PCR to further add Illumina adaptors and barcodes. Amplified libraries were gel extracted, quantified by a Qubit dsDNA HS assay (Thermo Fisher Scientific) and subject to single-end sequencing on an Illumina NextSeq with Read 1 75 cycles, Index 1 8 cycles and Index 2 8 cycles. PAMs were extracted and Weblogos depicting depleted PAMs were visualized using a custom Python script (Altae-Tran, H. et al. (2021).

### In Vitro Cleavage Assays

Double-stranded DNA (dsDNA) substrates were produced by PCR amplification of pUC19 plasmids containing the target sites and the TAM sequences. Cy3 and Cy5-conjugated DNA oligonucleotides (IDT) were used as primers to generate the labeled dsDNA substrates. Single-stranded DNA (ssDNA) substrates were ordered as Cy5.5-conjugated oligonucleotides (IDT). All ωRNA used in the biochemical assays was in vitro transcribed using the HiScribe T7 Quick High Yield RNA Synthesis kit (NEB) from the DNA templates purchased from Twist Biosciences. Target cleavage assays performed with AwalscB contained 10 nM of DNA substrate, 1 μM of protein, and 4 μM of ωRNA in a final 1×reaction buffer of 20 mM HEPES pH 7.5, 50 mM NaCl, and 5 mM MgCl₂. Assays were allowed to proceed at 37° C. for 1 hour, then briefly shifted to 50° C. for 5 min, and immediately placed on ice to help relax the RNA structure prior to RNA digestion. Reactions were then treated with RNase A (Qiagen), and Proteinase K (NEB), and purified using a PCR cleanup kit (Qiagen). DNA was resolved by gel electrophoresis on Novex 10% TBE (dsDNA substrates), 6% TBE-Urea (dsDNA substrates), and 15% TBE-Urea (ssDNA substrates) polyacrylamide gels (Thermo Fisher Scientific). Target cleavage assays performed with CRISPR-associated IscB RNPs were performed similarly, except that 450 nM RNP replaced the protein and the ωRNA, and the reactions were incubated for 1.5 hours at 37° C. Cleaned reactions were then run on a 4% agarose E-gel (Thermo Fisher Scientific). Target cleavage assays with AmaTnpB were performed with 1 μM of protein, 3 μM of ωRNA and 10 nM substrate, purified as described, and visualized on 2% agarose E-gels (dsDNA substrates) or 10% TBE-Urea polyacrylamide gels (ssDNA substrates).

For kinetic analysis of AwalscB or AmaTnpB activity, cleavage reactions were quenched with 11 mM of EDTA at each time point prior to cleanup. For screening metals, MgCl₂ was eliminated from the reaction buffer, while 2 mM of EDTA, and 7 mM of the indicated metal were added. Collateral cleavage assays for AwalscB were performed using 10 nM of unlabeled ds/ssDNA substrate along with 10 nM of Cy5.5-labeled collateral ssDNA substrate, and the reactions were allowed to proceed for 3 hours at 37° C. Cleavage of the labeled nontargeted ssDNA was then assessed on a 15% TBE-Urea polyacrylamide gel. Collateral cleavage assays for AmaTnpB were performed with 10 nM of unlabeled ds/ssDNA substrate and 10 nM of Cy5.5-labeled collateral ssDNA substrate for 1 hour at 60° C. and the resulting cleavage was visualized on a 10% TBE-Urea gel.

Single-stranded RNA (ssRNA) substrates were in vitro transcribed and labeled with pCp-Cy5 (Jena Bioscience) on their 3′ end. For the 3′ end labeling, 50 pmol of ssRNA was incubated with 100 pmol of pCp-Cy5 and 50 U of T4 RNA ligase 1 (NEB) in 50 mM Tris pH 7.8, 10 mM MgCl₂, 10 mM DTT, 2 mM ATP, and 10% DMSO at 4° C. for 40 hours. Labeling reactions were quenched with 20 mM EDTA and purified using an RNA Clean and Concentrator-25 kit (Zymo). ssRNA cleavage assays were performed similarly to the DNA cleavage assays, quenched with 19 mM EDTA at the end of the reactions, treated with Proteinase K, and visualized on a 6% TBE-Urea polyacrylamide gel.

For all experiments, all conditions were performed at least twice for replicability.

### Cell-Free Transcription Translation TAM Screen

IscB protein sequences were human codon optimized using the GenScript codon optimization tool, and IscB genes, TnpB genes with endogenous codon optimization, and ωRNA scaffolds were synthesized by Twist Biosciences. Transcription/translation templates were generated by PCR from custom synthesis products. Cell-free transcription/translation reactions were carried out using a PURExpress In Vitro Protein Synthesis Kit (NEB) as per the manufacturer's protocol with half-volume reactions, using 75 ng of template for the protein of interest, 125 ng of template for the corresponding ωRNA with a guide targeting the TAM library and 25 ng of TAM library plasmid. Reactions were incubated at 37° C. for 4 hours, then quenched by placing at 4° C. or on ice and adding 10 ug RNase A (Qiagen) and 8 units Proteinase K (NEB) each followed by a 5 min incubation at 37° C. DNA was extracted by PCR purification and adaptors were ligated using an NEBNext Ultra II DNA Library Prep Kit for Illumina (NEB) using the NEBNext Adaptor for Illumina (NEB) as per the manufacturer's protocol. Following adaptor ligation, cleaved products were amplified specifically using one primer specific to the TAM library backbone and one primer specific to the NEBNext adaptor with a 12-cycle PCR using NEBNext High Fidelity 2×PCR Master Mix (NEB) with an annealing temperature of 63° C., followed by a second 18-cycle round of PCR to further add the Illumina i5 adaptor. Amplified libraries were gel extracted, quantified by qPCR using a KAPA Library Quantification Kit for Illumina (Roche) on a StepOne Plus machine (Applied Biosystems/Thermo Fisher Scientific) and subject to single-end sequencing on an Illumina MiSeq with Read 1 80 cycles, Index 1 8 cycles and Index 2 8 cycles. TAMs were extracted and enrichment score for each TAM was calculated by filtering for all TAMs present more than once and normalizing to the TAM frequency in the input library subject to the same in vitro transcription/translation and quenching reactions. A position weight matrix based on the enrichment score was generated and Weblogos were visualized based on this position weight matrix using a custom Python script (Altae-Tran, H. et al. (2021).

### Cell-Free Transcription Translation Cleavage Assays

ωRNA templates were amplified from custom synthesis products as described and in vitro transcribed using a HiScribe T7 Quick High Yield RNA Synthesis Kit (NEB) with 150 ng DNA template in 2 μL, 2 μL T7 RNA Polymerase Mix (NEB) and 6.67 mM final concentration each NTP in 30 μL total volume reactions and purified with an RNA Clean & Concentrator-25 kit (Zymo). Protein sequences were amplified from custom synthesis products or locus plasmid templates. To generate targets, short oligos containing target and PAM sequences with appropriate overhangs were synthesized by Genewiz and cloned into corresponding backbone plasmids by Golden Gate or restriction-ligation cloning. Dye-conjugated primers for generating labeled linear targets with dyes as annotated were synthesized by IDT and linear targets were amplified from target plasmids by PCR using Q5 Hot Start High Fidelity 2×Master Mix (NEB) as per the manufacturer's protocol.

Cell-free transcription/translation reactions were carried out using a PURExpress In Vitro Protein Synthesis Kit (NEB) as per the manufacturer's protocol with half-volume reactions using 75 ng of template for the protein of interest and a final concentration of 1 μM in vitro-transcribed ωRNA to cap the possible final RNP concentration. Reactions were incubated at 37° C. for 4 hours to allow for RNP formation, then placed on ice to quench in vitro transcription/translation. 50-100 ng of target substrate was then added and the reactions were incubated at the specified temperature for 1 additional hour. Reactions were then quenched by adding 10 ug RNase A (Qiagen) and 8 units Proteinase K (NEB) each followed by a 5 min incubation at 37° C. DNA was extracted by PCR purification and run on either 10% or 6% Novex TBE-Urea gels, or 10% Novex TBE gels (Thermo Fisher Scientific) as per the manufacturer's protocols, as specified in figures. Gels were stained with 1×SYBR Gold (Thermo Fisher Scientific) where specified for 10-15 min and imaged on a ChemiDoc imager (BioRad) with optimal exposure settings. Each condition was performed twice for replicability.

### Mammalian Cell Culture and Transfection

Mammalian cell culture experiments were performed in the HEK293FT line (American Type Culture Collection (ATCC)) grown in Dulbecco's Modified Eagle Medium with high glucose, sodium pyruvate, and GlutaMAX (Thermo Fisher), additionally supplemented with 1× penicillin-streptomycin (Thermo Fisher), 10 mM HEPES (Thermo Fisher), and 10% fetal bovine serum (VWR Seradigm). All cells were maintained at confluency below 80%.

All transfections were performed with Lipofectamine 2000 (Thermo Fisher). Cells were plated 16-20 hours prior to transfection to ensure 90% confluency at the time of transfection. For 96-well plates, cells were plated at 20,000 cells/well, and for 24-well plates, cells were plated at 100,000 cells/well. For each well on the plate, transfection plasmids were combined with OptiMEM I Reduced Serum Medium (Thermo Fisher) to a total of 25 μL. Separately, 23 μL of OptiMEM was combined with 2 μL of Lipofectamine 2000. Plasmid and Lipofectamine solutions were then combined and pipetted onto cells.

### Mammalian Lysate Cleavage Assays

Human codon-optimized IscB genes were cloned into a CMV expression backbone by Gibson assembly using 2×Gibson Assembly Master Mix (NEB) to generate pCMV-SV40 NLS-IscB protein-nucleoplasmin NLS-3xHA constructs. 500 ng each protein expression plasmid was transfected in a separate well of a 24-well plate as described. After approximately 48 hours, cells were washed with 500 μL of Dulbecco's phosphate buffered saline (Sigma Aldrich). 50 μL ice-cold lysis buffer (20 mM HEPES 7.5, 100 mM KCl, 5 mM MgCl₂, 0.1% Triton-X 100, 5% glycerol, 1 mM DTT, 1×complete Protease Inhibitor Cocktail) was added, then cells were scraped from the plate, transferred to clean tubes and incubated on ice for 15 min. Cells were then sonicated in a cold water bath with amplitude 30 for 4 cycles of 10 s each. Lysate was then cleared by centrifugation at max speed for 20 min and supernatant was collected and either used fresh in assays or snap frozen in liquid nitrogen for later use. Labeled targets and in vitro-transcribed ωRNA was generated as described in “Cell-free transcription/translation cleavage assays” above.

To perform cleavage assays, 10 μL cell lysate was incubated with 1 μg in vitro-transcribed ωRNA or sgRNA, or no RNA for negative controls, and 100 ng target substrate in 1×NEBuffer 3.1 (NEB). Reactions were incubated at 37° C. for 1 hour, then quenched by adding 10 ug RNase A (Qiagen) and 8 units Proteinase K (NEB) each followed by a 5 min incubation at 37° C. DNA was extracted by PCR purification and run on a 4% Agarose E-gel EX (Thermo Fisher Scientific) as per the manufacturer's instructions and visualized on a ChemiDoc imager (BioRad).

### Sequencing of Cleavage Products

In vitro cleavage assays were performed as described. Purified reactions were subjected to a GLOE-seq library preparation protocol (Sriramachandran, A. et al. (2020), Mol. Cell. 78, 975-985.e7) as described using 2.5 μM adapter as input to the proximal adapter annealing step. The final amplification to add Illumina adapters and barcodes was performed with NEBNext High Fidelity 2×PCR Master Mix (NEB) with an annealing temperature of 63° C. for 15 s and 12 cycles. Libraries were subjected to paired-end sequencing using an Illumina MiSeq with Read 1 150 cycles, Read 2 150 cycles, Index 1 8 cycles and Index 2 8 cycles. Paired-end reads were mapped to the target substrate using BWA and 3′ ends were extracted and plotted using a custom Python script.

### Enzymatic Footprinting Assay

dsDNA substrate (191 bp) was produced by PCR amplification from a plasmid containing the target site and the TAM sequence. 10 pmol of dAwaIscB and 40 pmol of ωRNA were incubated in reaction buffer (20 mM HEPES pH 7.5, 50 mM NaCl, 10 mM MgCl₂, and 5% glycerol) at 37° C. for 30 min. Then, 0.1 pmol of DNA substrate was added and the reaction was allowed to proceed for another 30 min at 37° C. Next, 500 U of Exonuclease III (NEB) was added, the assay was incubated for an additional 10 min at 37° C. and quenched with 20 mM EDTA. As negative control, another reaction was run in parallel, in which ωRNA was excluded and the volume was replaced with water. After quenching, both reactions were briefly shifted to 50° C. for 5 min, then immediately placed on ice, treated with RNase A (Qiagen), and Proteinase K (NEB), and purified using a PCR cleanup kit (Qiagen). Purified reactions were subjected to a GLOE-seq library preparation protocol (Sriramachandran, A. et al. (2020), Mol. Cell. 78, 975-985.e7) as described using 2.5 μM adapter as input to both the proximal and distal adapter annealing steps. Libraries were amplified as described in the “Sequencing of cleavage products” section above and subjected to paired-end sequencing using an Illumina MiSeq with Read 1 100 cycles, Read 2 100 cycles, Index 1 8 cycles and Index 2 8 cycles. Paired-end reads were mapped to the target substrate using BWA and 3′ ends were extracted and plotted using a custom Python script (Altae-Tran, H. et al. (2021), Zenodo (2021).

### Mammalian Genome Editing

ωRNA scaffold backbones were cloned into a pUC19-based human U6 expression backbone by Gibson Assembly. For initial testing, 12-guide libraries were cloned in a pool mixing primers to add each of the 12 guides in in a given pool at equimolar ratios, and ωRNA scaffold backbones were subject to whole plasmid amplification with guide primers annealing to the U6 promoter and a second primer annealing to the start of the ωRNA scaffold using Phusion Flash High-Fidelity 2× Master Mix (Thermo Fisher Scientific). PCR products were gel extracted and eluted in 30 μL, then blunt-end ligated to circularize by addition of 5 units T4 PNK (NEB), 200 units T4 DNA Ligase (NEB) and final 1× T4 DNA Ligase Buffer (NEB) and incubation for 1.5 h at room temperature prior to transformation in Stb13 chemically competent E. coli (NEB). For individual guide constructs, oligos with appropriate overhangs were synthesized by Genewiz, annealed and phosphorylated using T4 PNK (NEB) and cloned into ωRNA backbones by restriction-ligation cloning. Protein expression constructs were cloned as described in “Mammalian lysate cleavage assays” above.

Prior to testing individual guides, each tested IscB protein was screened for activity in HEK293FT using a pool of 12 guides cloned as described. This was done to more rapidly assess many guides for a given protein for a broader survey of possible guides, as Applicants had no prior knowledge of the effect of guide properties such as sequence or genomic location may affect the ability of IscB to generate indels in eukaryotic cells. For this 12-guide pooled initial screening of IscB proteins, 800 ng protein expression construct and 1200 ng of the corresponding guide pool with corresponding ωRNA scaffold were transfected in one well of a 24-well plate as described. After 60-72 hours, genomic DNA was harvested by washing the cells once in 1×DPBS (Sigma Aldrich) and dry trypsinizing cells using TrypLE (Thermo Fisher Scientific). Trypsinized cells were collected in 1 mL 1×DPBS and pelleted by centrifugation at 300×g at 4° C. for 5 min. The supernatant was removed and cells were resuspended in 50 μL QuickExtract DNA Extraction Solution (Lucigen) and cycled at 65° C. for 15 min, 68° C. for 15 min then 95° C. for 10 min to lyse cells. 2.5 μL of lysed cells were used as input into each PCR reaction. Amplification of each region targeted by a guide in a given guide pool was performed individually. Insertion/deletion (indel) frequency was analyzed using CRISPResso2 (Clement, K. et al. (2019), Nat. Biotechnol. 37, 224-226). Given the low frequency of indel events with IscBs, in order to eliminate noise from PCR and sequencing error, only indels with at least 2 reads or more than 1 base inserted or deleted were counted towards reported indel frequencies. The allele frequency table generated by CRISPResso2 was used for manual verification of indels (i.e. both insertions and deletions occurring, occurrence of insertions and deletions of diverse sizes, not occurring in homonucleotide stretches), to determine if computed indels were due to PCR or sequencing error given that putative detected indels often occurred at frequencies similar to the detection threshold based on the number of cells input used in the experiment and the number of reads obtained from sequencing. Indel quantification results from the 12-guide pools are available in. Guides with manually verified indels from this pooled screen were assessed individually to validate indel formation as described below.

For individual guide sequences, 250 ng guide/ωRNA expression plasmid and 125 ng protein expression plasmid were transfected in each of 4 wells as biological replicates in a 96-well plate for each guide condition as described. After 60-72 hours, genomic DNA was harvested by washing the cells once in 1×DPBS (Sigma Aldrich) and adding 50 μL QuickExtract DNA Extraction Solution (Lucigen). Cells were scraped from the plates to suspend in QuickExtract and cycled at 65° C. for 15 min, 68° C. for 15 min then 95° C. for 10 min to lyse cells. 2.5 μL of lysed cells were used as input into each PCR reaction.

For library amplification, target genomic regions were amplified and with a 12-cycle PCR using NEBNext High Fidelity 2×PCR Master Mix (NEB) with an annealing temperature of 63° C. for 15 s, followed by a second 18-cycle round of PCR to add Illumina adapters and barcodes. The libraries were gel extracted and subject to single-end sequencing on an Illumina MiSeq with Read 1 300 cycles, Index 1 8 cycles, Index 2 8 cycles. Insertion/deletion (indel) frequency was analyzed using CRISPResso2 (Clement, K. et al. (2019), Nat. Biotechnol. 37, 224-226) as described. For individual guide/ωRNA experiments, to assess statistical significance, 2-tailed T-tests were performed using non-targeting guide/ωRNA conditions as a negative control (see Table 11).

### I. Tetrasporus Shotgun Sequencing

An agar slant of I. tetrasporus UTEX B 2012 was obtained from UTEX inoculated in Modified Bold 3N Medium (UTEX) and grown at 20° C. in a shaking incubator with 12 hour light/dark cycles. After 14 days, cultures were spun down and DNA was extracted using a DNeasy Plant Mini kit (Qiagen). Total DNA was tagmented using Tn5 and tagmentation reactions were quenched by addition of Buffer PB (Qiagen) and PCR purified. 10 μL purified DNA was used as input to PCR using 2×KOD HotStart Master Mix (Millipore Sigma) as per the manufacturer's instructions with an annealing temperature of 60° C. and 30 cycles to add Illumina adapters. Amplified libraries were gel extracted, quantified by qPCR using a KAPA Library Quantification Kit for Illumina (Roche) on a StepOne Plus machine (Applied Biosystems/Thermo Fisher Scientific) and subject to paired-end sequencing on an Illumina NextSeq with Read 1 150 cycles, Read 2 150 cycles, Index 1 8 cycles and Index 2 8 cycles. SPAdes-3.15.2 was used with the “isolate” option and kmer lengths of 21, 33, 55, 67 to create a contig level assembly from the reads (Bankevich, A. et al. (2012), J. Comput. Biol. 19, 455-477). BLASTn was then used to compare the final contigs containing ChlorIscB and IscB fragments with the published UTEX B 2012 chloroplast genome (GenBank accession KY407659.1) (Turmel, M. et al. (2017), Sci. Rep. 7, 994).

### Expression and Purification of TnpB Proteins

To purify the A. lobatus TnpB-2 protein in complex with the putative ωRNA of its native locus, an N-terminally His14-MBP tagged TnpB CDS and the corresponding downstream locus up to 80 bp beyond the end of predicted guide adaptor were cloned as a single piece downstream of a T7 promoter in a pET45b (+) vector. The AloTnpB-2 RNP was expressed and purified similarly to the CRISPR-associated IscB RNPs with the following modifications: (1) the lysis buffer, and the buffers A and B were supplemented with 40 mM imidazole, and 5 mM-mercaptoethanol, while MgCl₂ and DTT in all of the buffers were eliminated; (2) the RNP was purified on Ni-Sepharose 6 Fast Flow beads (GE Healthcare) instead of the Strep-Tactin resin; the elution buffer was supplemented with 300 mM imidazole and 5 mM β-mercaptoethanol, while MgCl₂, DTT and desthiobiotin were eliminated; (4) the His14-MBP solubility tag was kept attached to the RNP to provide stability.

To purify the A. macrosporangiidus TnpB, the native sequence was expressed from a pET45b (+) plasmid backbone, with a His14-MBP tag attached to its N-terminus via a TEV protease cleavage site. The AmaTnpB was expressed and purified similarly to AwalscB with the following modifications: (1) expression was performed in BL21 (DE3) cells (NEB) in the presence of 100 μg/ml ampicillin; (2) MgCl₂ was omitted from buffers C, D and E and the elution buffer; (3) TEV protease was added to the dialysis for solubility tag cleavage; (4) following dialysis, the protein was purified through a Resource S column against a gradient of 0.2-2M NaCl; (5) protein was concentrated up to 5 μM.

### Supplementary Text

### Extended Discussion of Hybrid CRISPR ωRNA and ωRNA Secondary Structure Analysis

Comparing the CRISPR-associated IscB ncRNA and ωRNA covariance model-based secondary structure predictions, Applicants found that all 10 base positions that were more than 97% conserved in the ωRNA were also highly conserved in the CRISPR-associated IscB ncRNA. Both the ωRNA and CRISPR-associated ncRNA contain a short 3-4 bp nexus, a conserved nexus hairpin, and a large region consisting of two interconnected multi-stem loops. The ωRNA additionally contains a hairpin that encompasses a Shine-Dalgarno (SD) sequence located approximately 10 bp upstream of the CDS start codon, implying that the ωRNA might be involved in the regulation of the translation of IscB. A statistically significant pseudoknot structure was predicted to exist in the ωRNA between the loop of one hairpin in the multi-stem loop region and the region directly downstream of the nexus, which Applicants termed the nexus pseudoknot hairpin. Examination of individual CRISPR-associated ncRNA sequences suggested the presence of a homologous pseudoknot structure. Scrambling the sequences found in the pseudoknot of the ncRNA disrupting the structure abolished the cleavage activity of the CRISPR-associated IscB. However, mutating the pseudoknot sequence while maintaining base pairing had no effect on cleavage activity, suggesting that the pseudoknot structure, but not necessarily sequence, was important for ncRNA function.

The majority of iscBs are associated with predicted ωRNA scaffolds matching the structure of the covariance model, but several large groups are not. To explore the diversity of the possible ωRNA scaffolds, Applicants iteratively built a set of profiles that span all major groups of ωRNAs associated with iscBs and isrBs, and found that different iscB/isrB clades were associated with structurally distinct ωRNAs (FIG. 9C). The structures differed by the number of hairpins in the multi-stem loop and guide adaptor regions, but the nexus pseudoknot was present in all major iscB- and isrB-associated ωRNAs. The IsrB ωRNAs additionally contained a second conserved pseudoknot between the guide adapter and a hairpin loop in the multi-stem loop region. Two of the three main IsrB ωRNA structures contained a third hairpin in the multi-stem loop region that forms the pseudoknot with the guide adaptor.

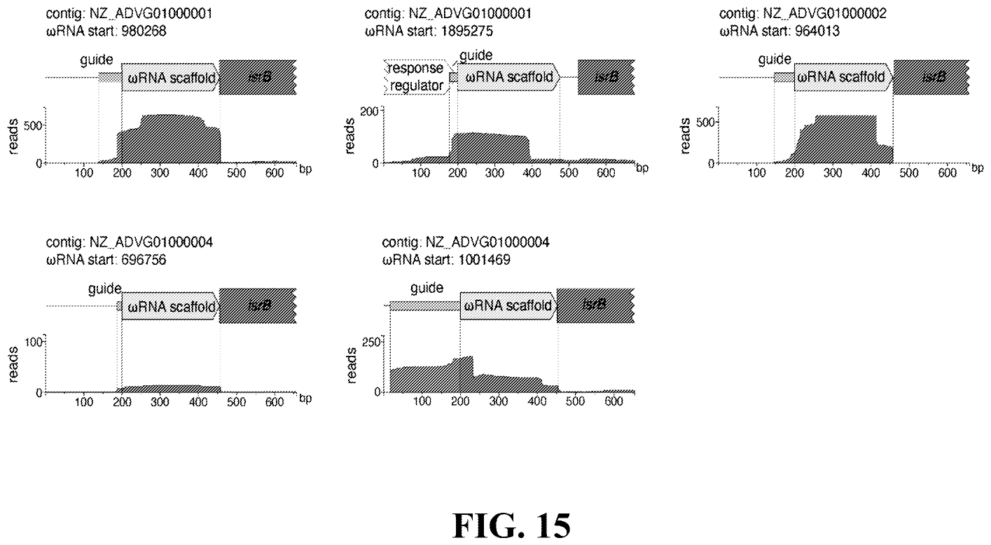

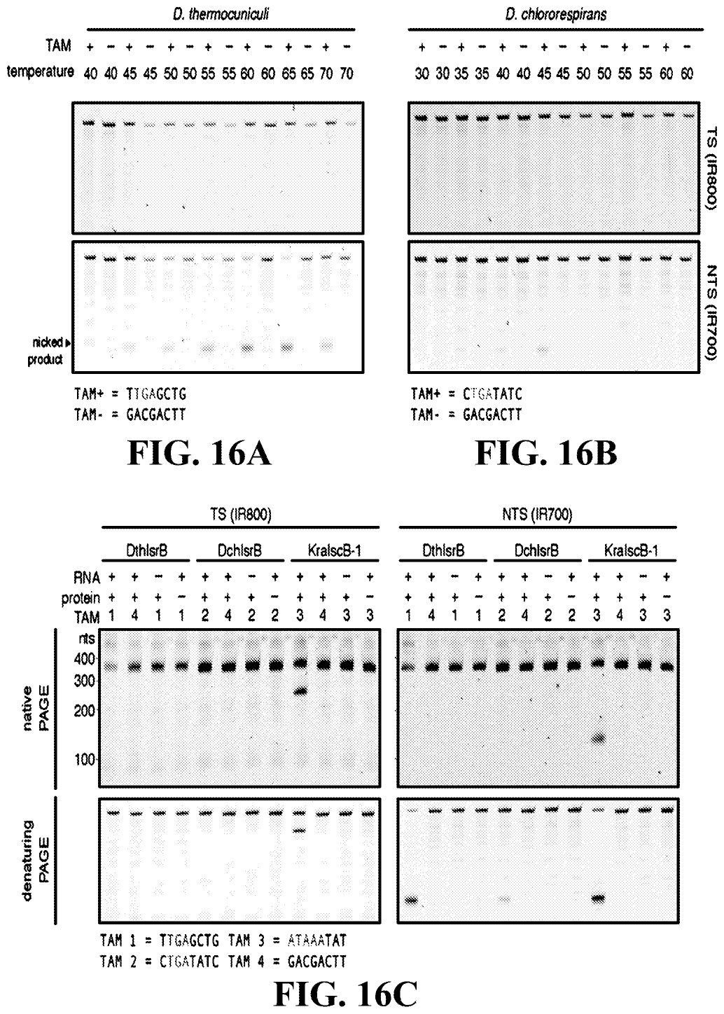

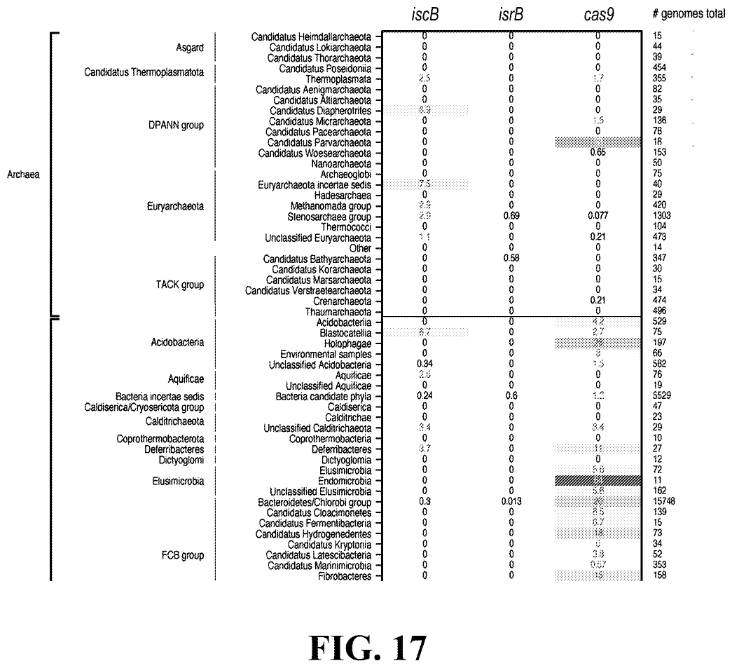

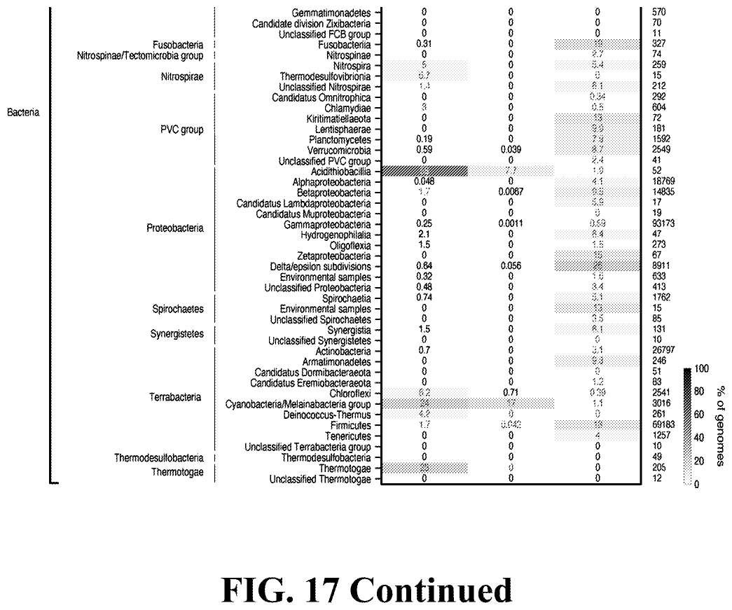

This guide adaptor pseudoknot was unique to IsrB ωRNAs and appears to have been lost contemporaneously with the insertion of the HNH domain (FIG. 15).

### Extended Phylogenetic Analysis and Discussion

To assess the robustness of our phylogenetic analyses and interpretations, Applicants describe in this section the additional analyses Applicants performed and the reasoning behind our phylogenetic conclusions.

To obtain maximally robust trees, Applicants used modified parameters for tree inference software. For all IQ-Tree 2 inferred trees, 5000 ultrafast bootstraps were performed with the bnni option to help reduce the impact of severe model violations on bootstrap support values (Hoang, D. et al. (2018), Mol. Biol. Evol. 35, 518-522).

Under this option, individual bootstrap trees are optimized using nearest neighbor exchange with hill-climbing on the likelihood function based on the bootstrap alignment. To improve the optimization performance of IQ-Tree 2 relative to default parameters, Applicants used nstop=500 (500 iterations of no improvement before terminating), with ninit=500 (500 initial trees), ntop=100 (100 top trees retained), and nbest=20 (20 best trees retained throughout the search and iteratively improved upon). For RAXML, Applicant sused 2000 rapid bootstraps for each tree to obtain accurate bootstrap values. For MrBayes, Applicants typically used 8-16 chains with a temperature difference between 0.01 and 0.025. Applicants also used 4-8 (resources permitting) independent runs to obtain a better estimate of convergence. Due to the occurrence of numerical overflow in MrBayes when computing likelihoods with a large number of taxa, Applicants employed a reaper script to restart from the last checkpoint when a numerical overflow was detected in any of the chains during the runs.

Applicants analyzed multiple different alignments to ensure robustness of the phylogenetic conclusions. Focusing the alignments only on the core, highly conserved regions containing the catalytic sites provided for comparison of IscB, IsrB, and Cas9s, proteins that widely differ in size and domain composition. Furthermore, analysis of slowly evolving regions enables phylogenetic inferences to be made over long evolutionary larger timescales that encompass the evolution of entire protein families, such as IscB and Cas9. The presence of multiple decaying, inactivated variants of IscB, IsrB, and Cas9 with high sequence divergence from active forms rendered long branch attraction a potential issue (Lockhart, P. et al. (1996), Proc. Natl. Acad. Sci. U.S.A 93, 1930-1934). Therefore, all inactivated proteins were removed from the RuvC/BH and RuvC/BH/HNH alignments to ensure the phylogeny tracked the evolution of active nucleases with the maximum possible accuracy, avoiding the artifacts caused by mixing active and inactive nucleases that evolve with substantially different rates. The evolution of Cas9 from IscB through the intermediate CRISPR-associated IscB cluster 2089 was supported by all inferred trees, and the evolution of IscB from IsrB is also strongly supported. However, to ensure robustness of these interpretations, Applicants considered numerous phylogenetic parameters, compared Bayesian and maximum likelihood methods, and also assessed the effect of taxa sampling (FIGS. 10C-D, 20, 21A-B).

Next, Applicants tested whether the main assumptions of phylogenetic inference held for the alignments used for our phylogenetic analyses. In some cases, the stationarity condition did not hold for the default alignment, prompting additional modifications to reduce the extent of stationarity violation. The stationarity violations detected for RuvC/BH/HNH alignments were mostly resolved by removing Cas9s of subtype II-B (prompted by the unstable placement of the major II-B Cas9 clade in the phylogenetic trees) and were completely eliminated by removing all Cas9s except for the earliest forms that evolved from IscB. This improvement in the tree quality suggests different evolutionary regimes for the core regions of IscB/IsrB (the RuvC, BH, and HNH domains) are substantially different in IscB/IsrBs and the homologous regions of the late, specialized Cas9s (II-B, II-C, tnpA-associated II-C, and the majority of II-A). Such a difference in evolutionary pressures is likely to stem from the shift in biological functionality between 22 systems (IscB/IsrB) and CRISPR-Cas systems (Cas9). Applicants performed additional phylogenetic analyses using these two modified alignments in order to assess the consistency of the inferences. The origin of Cas9 from the IscB cluster 2089 was consistently reproduced in these analyses (FIGS. 10, 21A,C).

Due to the removal of clusters that included inactivated nucleases, some CRISPR-associated IscBs were removed. For experimental characterization, Applicants considered all CRISPR-associated clusters regardless of whether or not they were included in the main phylogenetic analyses. In particular, IscB cluster 34507 contained mostly inactivated variants. Applicants studied IscBs from this cluster experimentally, reasoning that the high level of inactivation implies that the active members of this cluster are likely to have a high level of activity that could render them toxic to the host cell resulting in selection driving inactivation. To determine the likely position of this cluster in the full phylogenetic tree, Applicants performed an additional phylogenetic analysis of full IscB proteins (excluding the C-termini that aligned poorly). Applicants found that the experimentally studied CRISPR associated IscBs belonged to a small clade of CRISPR-associated IscBs that includes 4 non-redundant, CRISPR-associated loci. This analysis also yielded additional information on the relationship between IscB and Cas9. Because the alignment used for this analysis consisted of all IscBs and Cas9 1261 and Cas9_665 (two previously identified early Cas9 clusters from II-D), Applicants were able to construct a MAFFT-einsi alignment that spanned the entire length of these proteins (except for the C-terminal and the PLMP domain that is missing in Cas9). The phylogenetic analysis based on this alignment incorporated information from all homologous regions of IscB and Cas9 rather than the core region only. Clusters lacking coverage to any of the catalytic domains were removed; however, clusters including inactive nucleases were retained in the analysis. Positions in the alignment with more than 75% gaps were removed, and the tree was inferred using IQ-Tree 2 with the modified parameters described above. The exclusion of additional Cas9s in this analysis could potentially skew the placement of the Cas9 family. Nevertheless, the resulting tree supported CRISPR-associated IscB cluster 2089 as the ancestor of Cas9.

Phylogenetic symmetry tests showed that the main RuvC/BH/HNH alignment that included all Cas9s and IscBs likely violated the stationarity condition used by most phylogenetic methods (SRH conditions). Similarly, phylogenetic symmetry tests show that the high resolution (individual proteins as opposed to cluster representatives) codon DNA sequence alignment of early cas9s also likely violates the stationarity condition. Applicants considered a number of methods to assess the validity of the phylogenetic trees inferred using these alignments. For the early Cas9 tree, Applicants compared the tree inferred from the amino acid sequence (IQ-Tree 2, WAG+F+I+G4 model) to the tree inferred from the underlying DNA codon sequences (GTR+G4). The two trees were closely congruent with respect to the transition from IscB to Cas9 although despite the DNA tree inference only involved the simple GTR substitution model that does not take codon level information into account.

To assess the generalizability of the phylogenetic conclusions, in addition to bootstrap analyses in all ML trees, Applicants also performed a sensitivity analysis of the RuvC/BH/HNH phylogenetic inferences. Specifically, Applicants considered the effect of random exclusion of taxa. To this end, Applicants explored a range of taxa dropout rates. For each dropout rate, r, Applicants selected 1000/(1-r) alignments with r taxa randomly dropped out. Because Applicants were interested predominantly in the topology surrounding early Cas9 evolution, Applicants ensured that IscB cluster 2089 (putative Cas9 modern ancestor) and early Cas9 clusters (Cas9_1261, Cas9_665, Cas9_1079, Cas9_849) were retained in every alignment. Applicants then obtained inference for each of these taxon subsampled alignments. All trees were rooted on the clade of IscBs considered to be ancestral, that is evolved from IsrB, as inferred from the RuvC/BH tree. Next, various features (such as percentage of trees where Cas9 is monophyletic) were summarized across all trees for each dropout rate (FIG. 10). Error bars (95% confidence intervals) for the estimates were computed using 2000 bootstraps over the tree samples. Due to the computationally intense nature of ML inference for alignments with many taxa, Applicants limited this dropout analysis to FastTree2 (as opposed to IQ-Tree 2) based phylogenetic inference. The results of this analysis supported that Cas9_1261, Cas9_665 were the oldest Cas9 groups, and that all Cas9s likely originated from the ancestors of these two clusters. Moreover, the tree supported that even with taxon dropout rates of up to 50%, most of the resulting trees supported the CRISPR-associated IscB cluster 2089 as the ancestor to all extant Cas9s.

Applicants next considered whether or not the coarse graining of proteins (specifically the use of cluster representatives), as opposed to including all non-redundant sequences (at 90% redundancy), affected the phylogenetic inference. Applicants curated two “high resolution” alignments consisting of all non-redundant proteins of interest (Cas9/IscB) from clusters related to the early evolution of Cas9. One version of this alignment consisted of full protein alignments (using MAFFT-einsi) with the REC domains and PLMP domains removed. Positions with 75% or more gaps were removed. Another alignment consisted of translation aligned DNA codons for the ORFs, again removing REC domains and PLMP domains. Highly gapped codons triplet positions (sets of 3 positions) were removed using BMGE (Criscuolo, A. et al. (2010), BMC Evol. Biol. 10, 210). The resulting alignments were analyzed using IQ-Tree 2 and MrBayes. As the DNA alignment suffered from severe violation of the stationarity requirement for phylogenetic analysis, Applicants considered further using heterotachy models to account for changing modes of evolution throughout the underlying evolutionary tree. Adding a heterotachy-aware model did not change the inferred sequence of events in the evolution of early Cas9 from IscB. Phylogenetic analyses of the DNA sequences were agnostic to codons. Nevertheless, the two sets of alignments produced closely similar topologies for early events in Cas9 evolution. These topologies further agreed with previously identified topologies inferred using coarse grained clusters, suggesting that clustering was not detrimental to the inference of phylogenies.

Comparison of IscB/IsrB ωRNA phylogeny to the protein phylogenies can be useful for identifying co-evolution, and potentially could provide more information regarding the ancestry of Cas9. However, the structural motifs differed across the diversity of ωRNAs. Therefore, the main large-scale phylogenetic analysis across IscB/IsrB ωRNAs used a non-structured nucleotide alignment of representative ωRNAs that was produced using MAFFT-ginsi. Because the nexus pseudoknot is conserved in almost all ωRNAs, greater emphasis was given to this region by removing all sequences with low coverage to the nexus pseudoknot. An IQ-Tree 2 ML inference showed that IscB ωRNAs are a deep branch in the tree of ωRNAs, suggesting that IscB ωRNAs evolved on a single occasion from IsrB ωRNAs. Due to the high diversity of ωRNAs, the early Cas9_1261 and Cas9_665 tracrRNAs could not be included in the analysis for accurate placement because as the alignment across all RNAs failed to capture sufficient global sequence conservation. CMsearch of two tracrRNAs from early Cas9s (Cas9_1261 and Cas9_665) yielded significant hits to the G1a ωRNA profile (E-values 3.7e-8, 4. 1e-8 respectively), suggesting that at least part of the tracrRNA in early Cas9 systems originated from the IscB ωRNA (FIG. 11). The likely evolutionary link between ωRNAs and tracrRNAs further suggests that Cas9 evolved from IscB. To further address the question of whether or not early Cas9 tracrRNAs were derived from ωRNAs, Applicants constructed an accurate MAFFT-ginsi alignment of the early Cas9 tracrRNAs along with 25 closely related IscB ωRNAs, including the hybrid CRISPR/ωRNA from IscB cluster 2089. To account for the discrepancy between tracrRNAs and ωRNAs, Applicants concatenated the DR of the corresponding CRISPRs to the tracrRNA with a 4 bp poly-A tetraloop joining the two. Applicants performed a Bayesian phylogenetic analysis to address potential issues with the smaller number of taxa in this analysis. Applicants found that the combined DR/tracrRNAs likely evolved from IscB (2089) like ωRNAs, with high posterior probability (branch posterior 89%±1%). This result further suggests that all Cas9s evolved from the CRISPR-associated IscB cluster 2089.

Additional evidence from protein or RNA alignment comparisons were also useful in identifying the most likely ancestor of Cas9. On the sequence level, the Cas9_1261 and Cas9_665 representative sequences are more similar to the IscB cluster 2089 representative sequence in the split RuvC and HNH domains compared to other example IscBs. Moreover, the N-terminus of Cas9_1261 potentially contains fragments of the PLMP domain that is characteristic of IscB, suggesting that the PLMP domain gradually degraded during the transition to Cas9. Lastly, the 5′ end of the DRs from CRISPR-associated IscB cluster 2089 is closely similar to the 5′-terminal sequences of the DRs from Cas9_1261 and Cas9_665, suggesting further homology. All of these observations, in addition to the phylogenetic analyses, strongly suggest that CRISPR-associated IscB cluster 2089 is the ancestor of the earliest Cas9s. Accordingly, the sequence of events in the evolution of Cas9 was, first, the association of CRISPR arrays with IscB, either through duplication of the 5′ of the ωRNA or by insertion of the ωRNA into a CRISPR array. Then, REC-like sequences were inserted between the BH and RuvC-II, or possibly inside the BH, splitting it into two domains, the second of which remains in some early Cas9s. Then, the PLMP domain degraded from the lengthened IscB protein becoming Cas9, which subsequently became associated with the CRISPR adaptation module. Given the rarity of the early Cas9s, such as Subtype II-D, compared to IscB and other Cas9 subtypes, it appears likely that these systems containing small, simply organized Cas9s were outcompeted by the subsequently evolved, more efficient, larger Cas9s. From our phylogenetic analyses, the most likely candidate set of adaptation genes that became associated with Cas9 at first included Cas1, Cas2, and Cas4 genes. The association with Cas4 was then likely lost, forming Type II-C Cas9s, which subsequently gained additional associations to become II-A (associated with Csn2), II-B (re-association with Cas4), and tnpA-associated II-C(re-associated with Y1 recombinases).

### Extended Discussion of Potential Biological Functions of IS200 605 Endonucleases.

The biological functions of 2 systems remain unknown, but several hypotheses appear to be compatible with available evidence.

First, IscB, IsrB, and TnpB could mediate RNA-guided TnpA-catalyzed transposition while suppressing transposition independent of ωRNA. Our phylogenomic analysis together with previous observations (Kapitonov, V. et al. (2015), J. Bacteriol. 198, 797-807) indicate that iscB, isrB, and a subfamily of cas9 have all associated with the Y1 transposase tnpA of IS200/IS605 transposons on multiple, independent occasions (FIG. 9A) (Kapitonov, V. et al. (2015), J. Bacteriol. 198, 797-807). Moreover, the iscB, isrB and tnpB genes themselves are embedded in non-autonomous transposons of the IS200/IS605 superfamily. Integration events of ωRNA-containing transposons have been observed in locations with an ATGA or GTGA 3′ flanking sequences (Weinberg, Z. (2009), Nature 462, 656-659), which is a common TAM sequence for IscB proteins (FIG. 48). Many IscB TAMs contain start and stop codons, such that IscB targeting could mediate transposon insertion at the beginning or end of operons, allowing the transposon to exploit existing transcription initiation and termination signals. Together, these observations suggest the possibility that IscB, IsrB and TnpB are functionally analogous to CRISPR-associated transposases (CAST complexes) (Strecker, J. et al. (2019), Science 365, 48-53; Klompe, S. et al. (2019), Nature 571, 219-225), which mediate RNA-guided transposition of Tn7-like transposons. However, in contrast to CAST effectors such as Cas12k, an inactivated TnpB derivative (Strecker, J. et al. (2019), Science 365, 48-53), the great majority of IscB, IsrB and TnpB are predicted to be active nucleases. The role of the nuclease activity of these proteins in RNA-guided transposition (if any) remains to be studied experimentally. One possibility is that these nucleases eliminate aberrant DNA structures emerging upon RNA-guided transposon insertion.

An orthogonal hypothesis is that these nucleases, together with the ωRNA, are a distinct variety of toxins, to which TnpA is the antitoxin. Such toxins would kill cells lacking the inserted IS200/605 locus, thus making the host addicted to the IS200/605 transposase and ensuring that the non-autonomous transposons encoding IscB/TnpB also maintain their transposition capacity.

In addition to its direct role in transposition, TnpA might function to acquire guides for 22 systems, analogously to the Cas1 protein that is thought to have evolved from the transposase of the casposons, a distinct class of self-synthesizing transposons (Krupovic, M. et al. (2014), BMC Biol. 12, 1-12). The high sequence identity between the ωRNA transposon end-overlapping regions in both iscB and mpB loci in K. racemifer suggests that, at least in this bacterium, some IscBs and TnpBs share a mechanism for acquiring guide sequences. More generally, loci encoding complete 22 systems or standalone ωRNAs could be mobilized and moved to new locations by TnpA, thereby associating with new, diverse flanking sequences that become guides.

### References for Extended Discussion of Supplemental Materials

1. F. Zhang, Development of CRISPR-Cas systems for genome editing and beyond. Quarterly Reviews of Biophysics. 52 (2019).
2. F. Hille, H. Richter, S. P. Wong, M. Bratovič, S. Ressel, E. Charpentier, The biology of CRISPR-Cas: Backward and forward. Cell. 172, 1239-1259 (2018).
3. V. V. Kapitonov, K. S. Makarova, E. V. Koonin, ISC, a novel group of bacterial and Archaeal DNA transposons that encode Cas9 homologs. J. Bacteriol. 198, 797-807 (2015).
4. P. Siguier, E. Gourbeyre, M. Chandler, Bacterial insertion sequences: their genomic impact and diversity. FEMS Microbiol. Rev. 38, 865-891 (2014).
5. S. Shmakov, A. Smargon, D. Scott, D. Cox, N. Pyzocha, W. Yan, O. O. Abudayyeh, J. S. Gootenberg, K. S. Makarova, Y. I. Wolf, K. Severinov, F. Zhang, E. V. Koonin, Diversity and evolution of class 2 CRISPR-Cas systems. Nat. Rev. Microbiol. 15, 169-182 (2017).
6. B. Zetsche, J. S. Gootenberg, O. O. Abudayyeh, I. M. Slaymaker, K. S. Makarova, P. Essletzbichler, S. E. Volz, J. Joung, J. van der Oost, A. Regev, E. V. Koonin, F. Zhang, Cpf1 is a single RNA-guided endonuclease of a class 2 CRISPR-Cas system. Cell. 163, 759-771 (2015).
7. E. Deltcheva, K. Chylinski, C. M. Sharma, K. Gonzales, Y. Chao, Z. A. Pirzada, M. R. Eckert, J. Vogel, E. Charpentier, CRISPR RNA maturation by trans-encoded small RNA and host factor RNase III. Nature. 471, 602-607 (2011).
8. I. Kalvari, E. P. Nawrocki, N. Ontiveros-Palacios, J. Argasinska, K. Lamkiewicz, M. Marz, S. Griffiths-Jones, C. Toffano-Nioche, D. Gautheret, Z. Weinberg, E. Rivas, S. R. Eddy, R. D. Finn, A. Bateman, A. I. Petrov, Rfam 14: expanded coverage of metagenomic, viral and microRNA families. Nucleic Acids Res. 49, D192-D200 (2021).
9. Z. Weinberg, J. Perreault, M. M. Meyer, R. R. Breaker, Exceptional structured noncoding RNAs revealed by bacterial metagenome analysis. Nature. 462, 656-659 (2009).
10. M. Jinek, K. Chylinski, I. Fonfara, M. Hauer, J. A. Doudna, E. Charpentier, A programmable dual-RNA-guided DNA endonuclease in adaptive bacterial immunity. Science. 337, 816-821 (2012).
11. G. Gasiunas, R. Barrangou, P. Horvath, V. Siksnys, Cas9-crRNA ribonucleoprotein complex mediates specific DNA cleavage for adaptive immunity in bacteria. Proc. Natl. Acad. Sci. U.S.A 109, E2579-86 (2012).
12. G. Gasiunas, J. K. Young, T. Karvelis, D. Kazlauskas, T. Urbaitis, M. Jasnauskaite, M. M. Grusyte, S. Paulraj, P.-H. Wang, Z. Hou, S. K. Dooley, M. Cigan, C. Alarcon, N. D. Chilcoat, G. Bigelyte, J. L. Curcuru, M. Mabuchi, Z. Sun, R. T. Fuchs, E. Schildkraut, P. R. Weigele, W. E. Jack, G. B. Robb, Č. Venclovas, V. Siksnys, A catalogue of biochemically diverse CRISPR-Cas9 orthologs. Nat. Commun. 11, 5512 (2020).
13. M. Jinek, F. Jiang, D. W. Taylor, S. H. Sternberg, E. Kaya, E. Ma, C. Anders, M. Hauer, K. Zhou, S. Lin, M. Kaplan, A. T. Iavarone, E. Charpentier, E. Nogales, J. A. Doudna, Structures of Cas9 endonucleases reveal RNA-mediated conformational activation. Science. 343, 1247997 (2014).
14. B. Q. Minh, H. A. Schmidt, O. Chernomor, D. Schrempf, M. D. Woodhams, A. von Haeseler, R. Lanfear, IQ-TREE 2: New Models and Efficient Methods for Phylogenetic Inference in the Genomic Era. Mol. Biol. Evol. 37, 1530-1534 (2020).
15. K. S. Makarova, Y. I. Wolf, J. Iranzo, S. A. Shmakov, O. S. Alkhnbashi, S. J. J. Brouns, E. Charpentier, D. Cheng, D. H. Haft, P. Horvath, S. Moineau, F. J. M. Mojica, D. Scott, S. A. Shah, V. Siksnys, M. P. Terns, C. Venclovas, M. F. White, A. F. Yakunin, W. Yan, F. Zhang, R. A. Garrett, R. Backofen, J. van der Oost, R. Barrangou, E. V. Koonin, Evolutionary classification of CRISPR-Cas systems: a burst of class 2 and derived variants. Nat. Rev. Microbiol. 18, 67-83 (2020).
16. K. A. Majorek, S. Dunin-Horkawicz, K. Steczkiewicz, A. Muszewska, M. Nowotny, K. Ginalski, J. M. Bujnicki, The RNase H-like superfamily: new members, comparative structural analysis and evolutionary classification. Nucleic Acids Res. 42, 4160-4179 (2014).
17. H. Nishimasu, F. A. Ran, P. D. Hsu, S. Konermann, S. I. Shehata, N. Dohmae, R. Ishitani, F. Zhang, O. Nureki, Crystal structure of Cas9 in complex with guide RNA and target DNA. Cell. 156, 935-949 (2014).
18. W. Bao, J. Jurka, Homologues of bacterial TnpB_IS605 are widespread in diverse eukaryotic transposable elements. Mob. DNA. 4, 12 (2013).
19. J. V. Gomes-Filho, L. S. Zaramela, V. C. da S. Italiani, N. S. Baliga, R. Z. N. Vêncio, T. Koide, Sense overlapping transcripts in IS1341-type transposase genes are functional non-coding RNAs in archaea. RNA Biol. 12, 490-500 (2015).
20. Z. Weinberg, C. E. Lunse, K. A. Corbino, T. D. Ames, J. W. Nelson, A. Roth, K. R. Perkins, M. E. Sherlock, R. R. Breaker, Detection of 224 candidate structured RNAs by comparative analysis of specific subsets of intergenic regions. Nucleic Acids Res. 45, 10811-10823 (2017).
21. A. Hüttenhofer, P. Schattner, The principles of guiding by RNA: chimeric RNA-protein enzymes. Nat. Rev. Genet. 7, 475-482 (2006).
22. A. Schneider, A short history of guide RNAs: The intricate path that led to the discovery of a basic biological concept. EMBO Rep. 21, e51918 (2020).
23. E. V. Koonin, Evolution of RNA- and DNA-guided antivirus defense systems in prokaryotes and eukaryotes: common ancestry vs convergence. Biol. Direct. 12 (2017).
24. H. Altae-Tran, S. Kannan, F. Zhang, Code and processed data for: The widespread IS200/605 transposon family encodes diverse programmable RNA-guided endonucleases, Version 1.0, Zenodo (2021); <link to be inserted after official acceptance of manuscript>.
25. C. Camacho, G. Coulouris, V. Avagyan, N. Ma, J. Papadopoulos, K. Bealer, T. L. Madden, BLAST+: architecture and applications. BMC Bioinformatics. 10, 421 (2009).
26. K. Katoh, D. M. Standley, MAFFT multiple sequence alignment software version 7: improvements in performance and usability. Mol. Biol. Evol. 30, 772-780 (2013).
27. M. Steinegger, J. Söding, MMseqs2 enables sensitive protein sequence searching for the analysis of massive data sets. Nat. Biotechnol. 35, 1026-1028 (2017).
28. M. Steinegger, M. Meier, M. Mirdita, H. Vöhringer, S. J. Haunsberger, J. Söding, HH-suite3 for fast remote homology detection and deep protein annotation. BMC Bioinformatics. 20, 473 (2019).
29. S. R. Eddy, Accelerated Profile HMM Searches. PLoS Comput. Biol. 7, e1002195 (2011).
30. S. A. Shmakov, K. S. Makarova, Y. I. Wolf, K. V. Severinov, E. V. Koonin, Systematic prediction of genes functionally linked to CRISPR-Cas systems by gene neighborhood analysis. Proc. Natl. Acad. Sci. U.S.A 115, E5307-E5316 (2018).
31. A. B. Crawley, J. R. Henriksen, R. Barrangou, CRISPRdisco: An Automated Pipeline for the Discovery and Analysis of CRISPR-Cas Systems. CRISPR J. 1, 171-181 (2018).
32. D. H. Haft, B. J. Loftus, D. L. Richardson, F. Yang, J. A. Eisen, I. T. Paulsen, O. White, TIGRFAMs: a protein family resource for the functional identification of proteins. Nucleic Acids Res. 29, 41-43 (2001).
33. A. Delcher, Improved microbial gene identification with GLIMMER. Nucleic Acids Research. 27 (1999), pp. 4636-4641.
34. S. Naser-Khdour, B. Q. Minh, W. Zhang, E. A. Stone, R. Lanfear, The Prevalence and Impact of Model Violations in Phylogenetic Analysis. Genome Biol. Evol. 11, 3341-3352 (2019).
35. S. Kalyaanamoorthy, B. Q. Minh, T. K. F. Wong, A. von Haeseler, L. S. Jermiin, ModelFinder: fast model selection for accurate phylogenetic estimates. Nat. Methods. 14, 587-589 (2017).
36. M. N. Price, P. S. Dehal, A. P. Arkin, FastTree 2--approximately maximum-likelihood trees for large alignments. PLOS One. 5, e9490 (2010).
37. G. Altekar, S. Dwarkadas, J. P. Huelsenbeck, F. Ronquist, Parallel Metropolis coupled Markov chain Monte Carlo for Bayesian phylogenetic inference. Bioinformatics. 20, 407-415 (2004).
38. A. Stamatakis, RAXML version 8: a tool for phylogenetic analysis and post-analysis of large phylogenies. Bioinformatics. 30, 1312-1313 (2014).
39. R. Lorenz, S. H. Bernhart, C. H. zu Siederdissen, H. Tafer, C. Flamm, P. F. Stadler, I. L. Hofacker, ViennaRNA Package 2.0. Algorithms Mol. Biol. 6, 1-14 (2011).
40. E. Rivas, J. Clements, S. R. Eddy, A statistical test for conserved RNA structure shows lack of evidence for structure in lncRNAs. Nat. Methods. 14, 45-48 (2017).
41. E. P. Nawrocki, S. R. Eddy, Infernal 1.1:100-fold faster RNA homology searches. Bioinformatics. 29, 2933-2935 (2013).
42. Z. Weinberg, R. R. Breaker, R2R—software to speed the depiction of aesthetic consensus RNA secondary structures. BMC Bioinformatics. 12, 3 (2011).
43. J. Mistry, S. Chuguransky, L. Williams, M. Qureshi, G. A. Salazar, E. L. L. Sonnhammer, S. C. E. Tosatto, L. Paladin, S. Raj, L. J. Richardson, R. D. Finn, A. Bateman, Pfam: The protein families database in 2021. Nucleic Acids Res. 49, D412-D419 (2021).
44. C. Bland, T. L. Ramsey, F. Sabree, M. Lowe, K. Brown, N. C. Kyrpides, P. Hugenholtz, CRISPR recognition tool (CRT): a tool for automatic detection of clustered regularly interspaced palindromic repeats. BMC Bioinformatics. 8, 209 (2007).
45. F. Asnicar, G. Weingart, T. L. Tickle, C. Huttenhower, N. Segata, Compact graphical representation of phylogenetic data and metadata with GraPhlAn. Peer J. 3, e1029 (2015).
46. L. Maaten, G. Hinton, Visualizing Data using t-SNE. Journal of machine learning research. 9, 2579-2605 (2008).
47. P. G. Poličar, M. Stražar, B. Zupan, Embedding to reference t-SNE space addresses batch effects in single-cell classification. bioRxiv (2019), doi: 10.1101/671404.
48. P. J. Rousseeuw, Silhouettes: A graphical aid to the interpretation and validation of cluster analysis. J. Comput. Appl. Math. 20, 53-65 (1987).
49. R. J. G. B. Campello, D. Moulavi, J. Sander, in Advances in Knowledge Discovery and Data Mining (Springer Berlin Heidelberg, Berlin, Heidelberg, 2013), Lecture notes in computer science, pp. 160-172.
50. D. H. Parks, M. Imelfort, C. T. Skennerton, P. Hugenholtz, G. W. Tyson, CheckM: assessing the quality of microbial genomes recovered from isolates, single cells, and metagenomes. Genome Res. 25, 1043-1055 (2015).
51. P.-A. Chaumeil, A. J. Mussig, P. Hugenholtz, D. H. Parks, GTDB-Tk: a toolkit to classify genomes with the Genome Taxonomy Database. Bioinformatics (2019), doi: 10.1093/bioinformatics/btz848.
52. S. Frey, D. Görlich, A new set of highly efficient, tag-cleaving proteases for purifying recombinant proteins. J. Chromatogr. A. 1337, 95-105 (2014).
53. M. Martin, Cutadapt removes adapter sequences from high-throughput sequencing reads. EMBnet.journal. 17, 10-12 (2011).
54. B. Langmead, S. L. Salzberg, Fast gapped-read alignment with Bowtie 2. Nat. Methods. 9, 357-359 (2012).
55. A. M. Sriramachandran, G. Petrosino, M. Méndez-Lago, A. J. Schäfer, L. S. Batista-Nascimento, N. Zilio, H. D. Ulrich, Genome-wide Nucleotide-Resolution Mapping of DNA Replication Patterns, Single-Strand Breaks, and Lesions by GLOE-Seq. Mol. Cell. 78, 975-985.e7 (2020).
56. K. Clement, H. Rees, M. C. Canver, J. M. Gehrke, R. Farouni, J. Y. Hsu, M. A. Cole, D. R. Liu, J. K. Joung, D. E. Bauer, L. Pinello, CRISPResso2 provides accurate and rapid genome editing sequence analysis. Nat. Biotechnol. 37, 224-226 (2019).
57. A. Bankevich, S. Nurk, D. Antipov, A. A. Gurevich, M. Dvorkin, A. S. Kulikov, V. M. Lesin, S. I. Nikolenko, S. Pham, A. D. Prjibelski, A. V. Pyshkin, A. V. Sirotkin, N. Vyahhi, G. Tesler, M. A. Alekseyev, P. A. Pevzner, SPAdes: a new genome assembly algorithm and its applications to single-cell sequencing. J. Comput. Biol. 19, 455-477 (2012).
58. M. Turmel, C. Otis, C. Lemieux, Divergent copies of the large inverted repeat in the chloroplast genomes of ulvophycean green algae. Sci. Rep. 7, 994 (2017).
59. D. T. Hoang, O. Chernomor, A. von Haeseler, B. Q. Minh, L. S. Vinh, UFBoot2: Improving the Ultrafast Bootstrap Approximation. Mol. Biol. Evol. 35, 518-522 (2018).
60. P. J. Lockhart, A. W. Larkum, M. Steel, P. J. Waddell, D. Penny, Evolution of chlorophyll and bacteriochlorophyll: the problem of invariant sites in sequence analysis. Proc. Natl. Acad. Sci. U.S.A 93, 1930-1934 (1996).
61. A. Criscuolo, S. Gribaldo, BMGE (Block Mapping and Gathering with Entropy): a new software for selection of phylogenetic informative regions from multiple sequence alignments. BMC Evol. Biol. 10, 210 (2010).
62. J. Strecker, A. Ladha, Z. Gardner, J. L. Schmid-Burgk, K. S. Makarova, E. V. Koonin, F. Zhang, RNA-guided DNA insertion with CRISPR-associated transposases. Science. 365, 48-53 (2019).
63. S. E. Klompe, P. L. H. Vo, T. S. Halpin-Healy, S. H. Sternberg, Transposon-encoded CRISPR-Cas systems direct RNA-guided DNA integration. Nature. 571, 219-225 (2019).
64. M. Krupovic, K. S. Makarova, P. Forterre, D. Prangishvili, E. V. Koonin, Casposons: a new superfamily of self-synthesizing DNA transposons at the origin of prokaryotic CRISPR-Cas immunity. BMC Biol. 12, 1-12 (2014).
65. S. M. Crotty, B. Q. Minh, N. G. Bean, B. R. Holland, J. Tuke, L. S. Jermiin, A. V. Haeseler, GHOST: Recovering Historical Signal from Heterotachously Evolved Sequence Alignments. Syst. Biol. 69, 249-264 (2020).

### Example 3—Characterization of IsrB System Structure and Targeting Mechanism

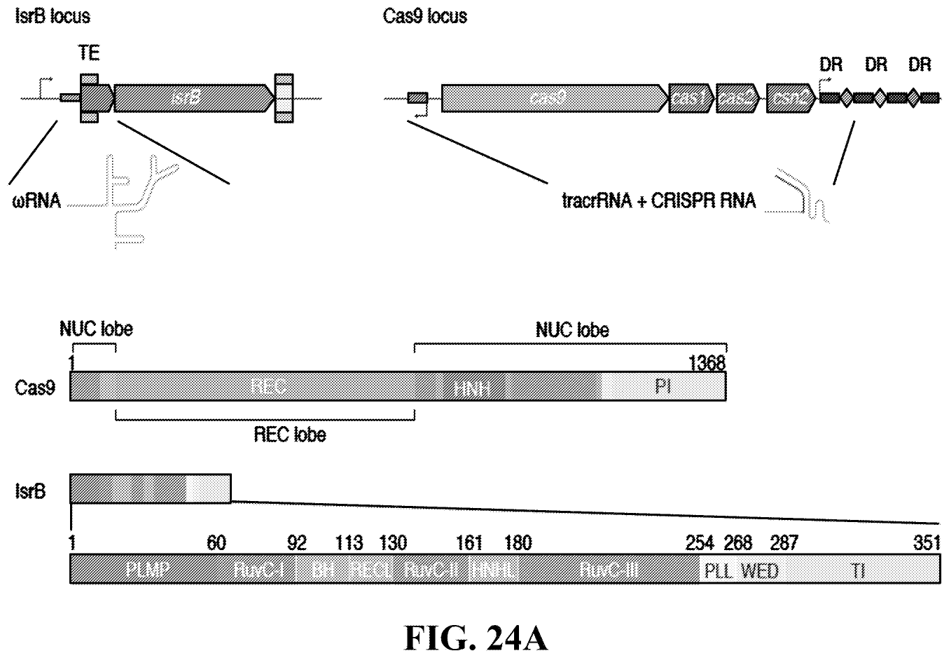

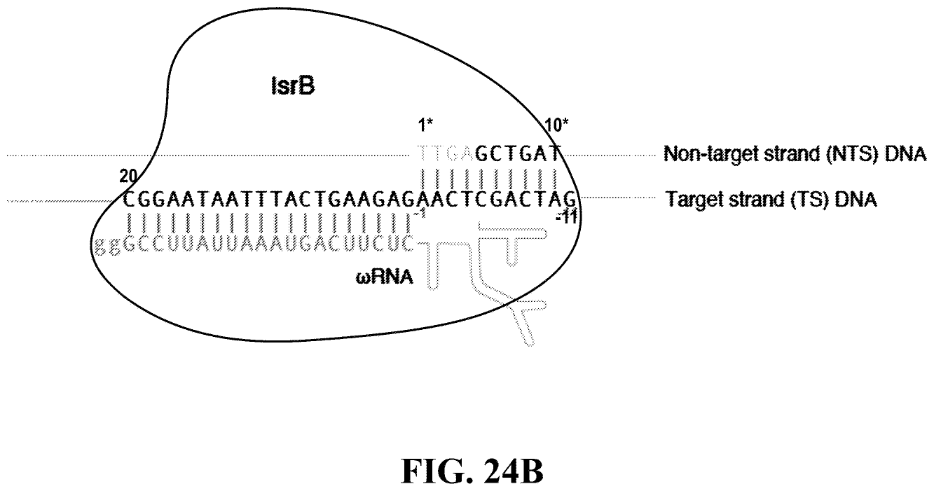

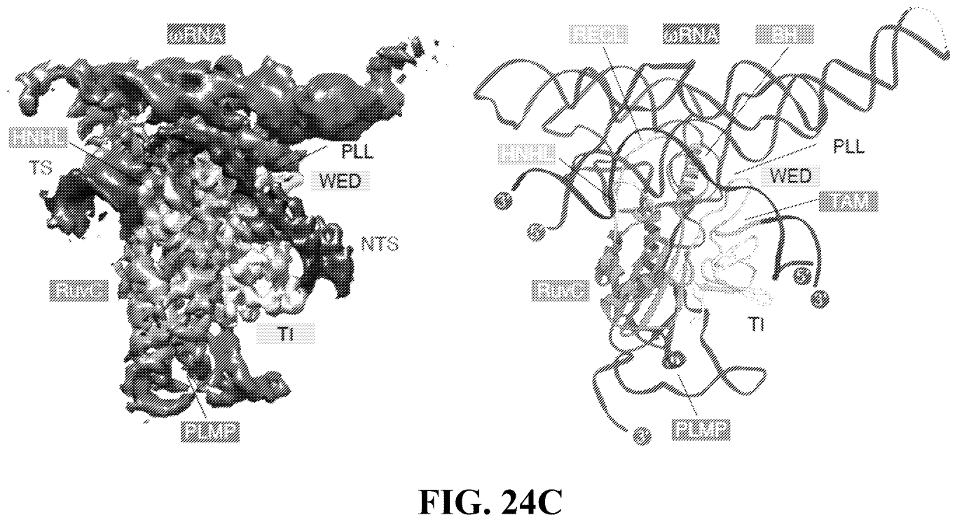

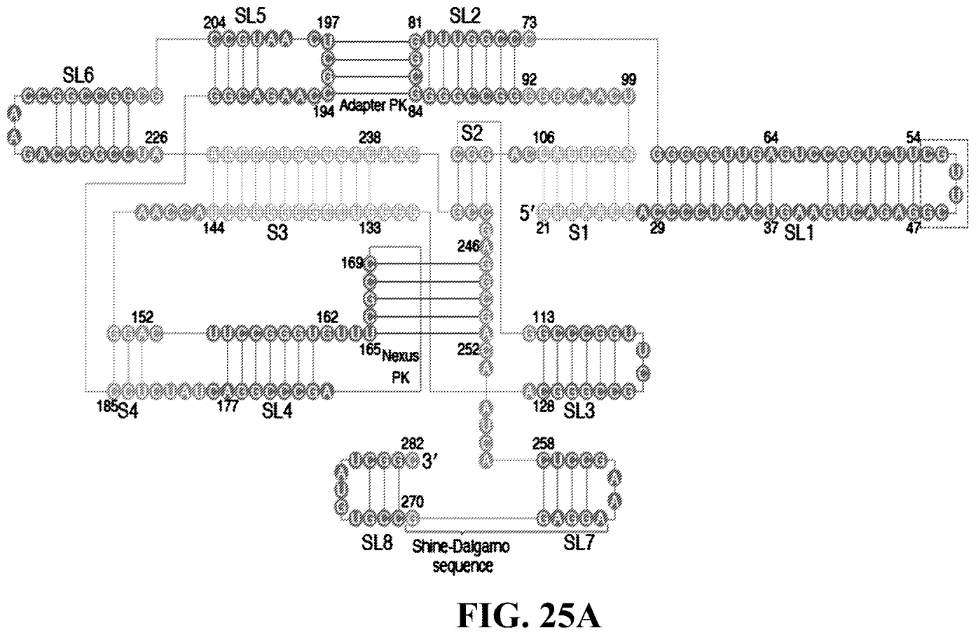

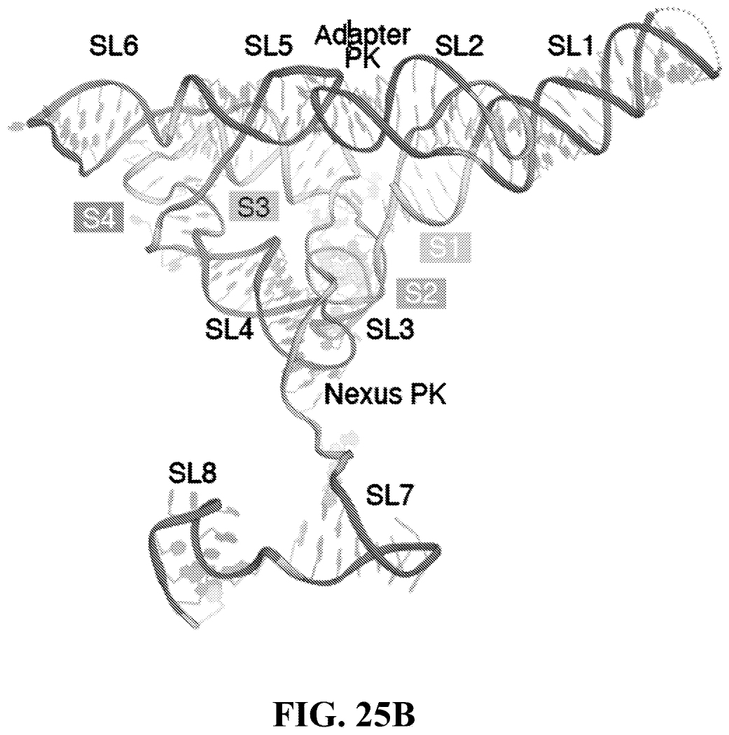

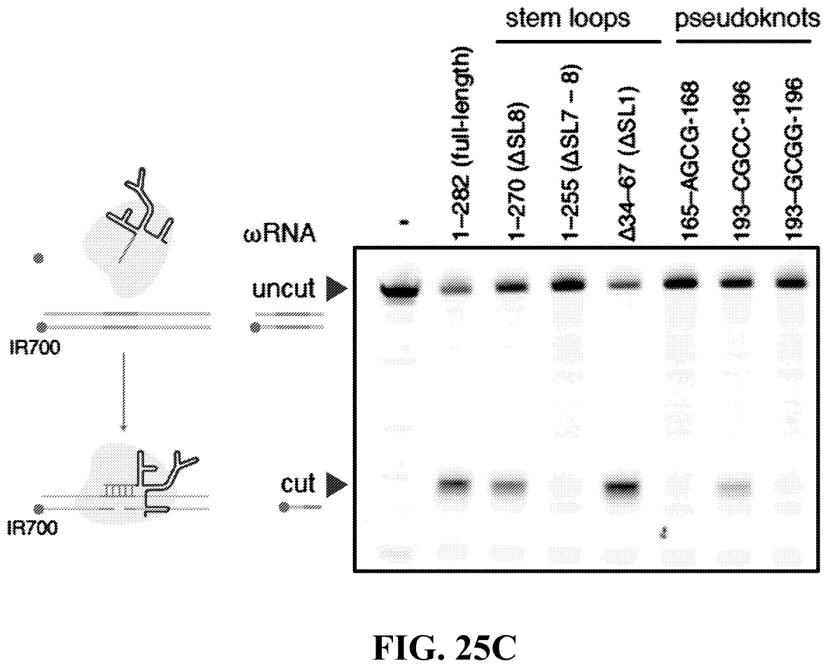

Recent work identified a new class of RNA-guided systems, termed OMEGA, which include IscB, the likely ancestor of Cas9, and the nickase IsrB, a relative of IscB lacking the HNH nuclease domain. (FIG. 23A). IsrB is only ˜350 amino acids long, but the small size of the protein is counterbalanced by a relatively large guide RNA (˜300-nt ωRNA) (FIG. 24B, 24C). The cryoelectron microscopy structure of IsrB in complex with ωRNA and target DNA is described in this example. The overall structure of the IsrB protein shares a common scaffold with Cas9. In contrast to Cas9, however, which uses a recognition (REC) domain to facilitate target selection, IsrB uses the ωRNA, part of which forms a complex ternary structure positioned analogously to the REC lobe. The structural analyses of IsrB and the ωRNA as well as comparisons to other RNA-guided systems highlight the functional interplay between protein architecture and RNA, advancing the understanding of the biology and evolution of these diverse systems. FIGS. 24A-24C depict example IsrB domain organization relative to Cas9 (24A), interaction with ωRNA in complex at the target strand and non-target strand of DNA (24B); and the 3D and ribbon diagram depiction of the IscB and ωRNA (24C). The Applicants further characterized the ωRNA structure (25A, 25B) and the nuclease activity of the IsrB system with full length ωRNA and ωRNA with edits to stem-loop 8 (SL8), SL7-8, SL1, or edits to pseudoknots at positions 165-168, or 193-196 (FIG. 25C).

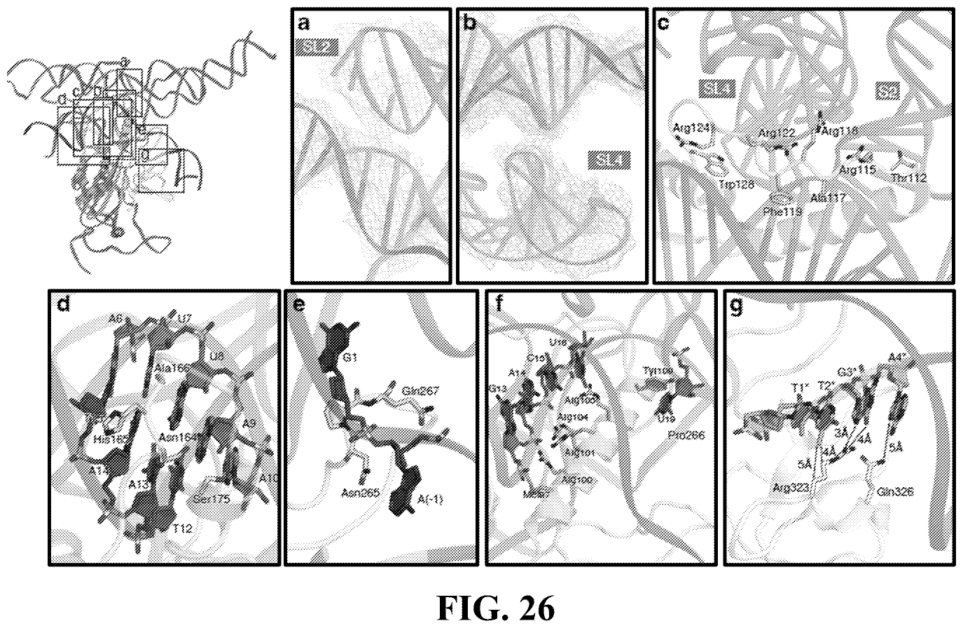

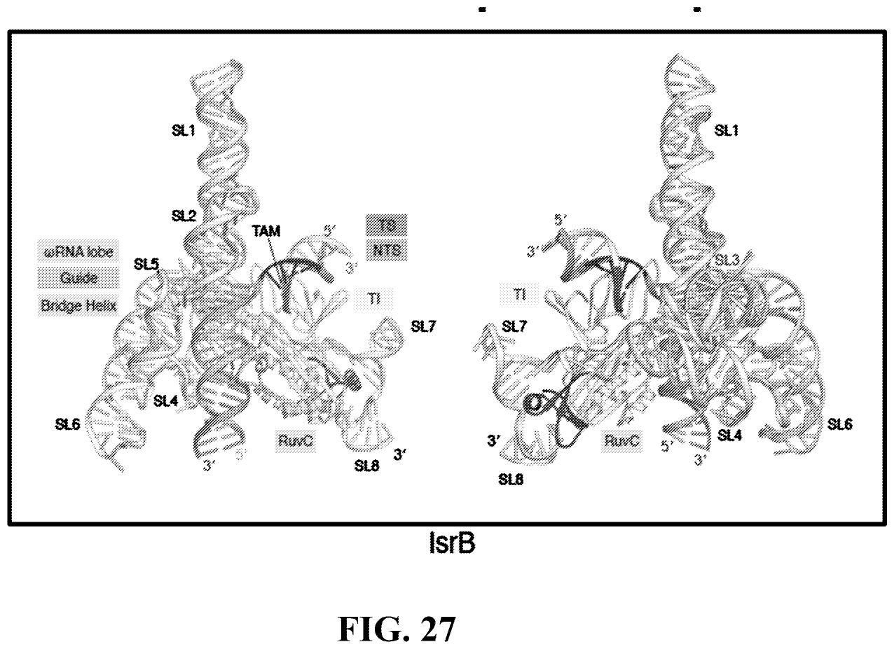

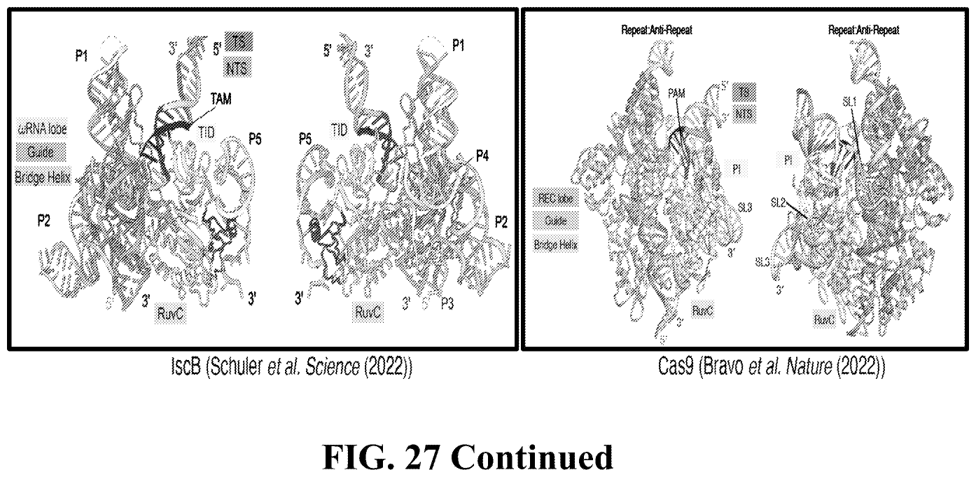

RNA-guide DNA targeting is depicted in FIG. 26, showing participating amino acid residues and nucleotide positions involved in the mechanism in aspects a-g from FIG. 26. FIG. 27 includes a snapshot of the IscrB species, with relevant comparison to IscB at FIG. 1E of Schuler et al., Science (2022) and Cas9 in Bravo et al. Nature (2022), incorporated herein by reference.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A non-naturally occurring, engineered composition comprising:
a) an IsrB polypeptide comprising a split Ruv-C nuclease domain comprising RuvC-I, RuvC-II, and RuvC-III subdomains, and
b) one or more ωRNA molecules, wherein each ωRNA molecule comprises a scaffold and a reprogrammable spacer sequence (e.g., comprising a spacer of 10 nucleotides to 150 nucleotides in length, preferably 12 to 50 nt, more preferably 15 and 45 nt in length), wherein each ωRNA molecule forms a complex with the IsrB polypeptide and directs sequence-specific binding of the complex to a target sequence on a target polynucleotide.

2. The composition of claim 1, wherein:
the IsrB polypeptide comprises a PLMP domain and optionally a conserved C-terminal Y domain;
the IsrB polypeptide does not comprise a HNH domain;
the IsrB polypeptide further comprises a bridge helix domain and optionally the bridge helix domain is located between the RuvC-I and RuvC-II subdomains;
the IsrB polypeptide comprises about 170 to about 700 amino acids; and/or
the IsrB polypeptide is catalytically inactive (“dIsrB”), optionally selected from Table X.

3-7. (canceled)

8. The composition of claim 1, wherein the ωRNA further comprises one or more chemical modifications; and/or wherein the complex recognizes a target adjacent motif (TAM) sequence 3′ of the target polynucleotide.

9. (canceled)

10. The composition of claim 1, comprising at least two ωRNA molecules, wherein the at least two ωRNA molecules target opposite stands of a double-stranded target polynucleotide, and wherein the complex comprises a nick on opposite stands either side of the target sequence; and/or further comprising a homologous recombination donor template comprising a donor sequence for insertion into the target polynucleotide.

11. (canceled)

12. (canceled)

13. A polynucleotide encoding the IsrB polypeptide and/or the ωRNA of claim 1.

14. A vector system comprising one or more vectors encoding the IsrB polypeptide and the ωRNA molecule of claim 1.

15. An isolated cell, or progeny thereof, comprising the composition of claim 1.

16. A method of contacting a target polynucleotide sequence in a cell, comprising introducing to the cell the composition of claim 1, and optionally, wherein the IsrB polypeptide and/or one or more nucleic acid components thereof are provided via one or more polynucleotides encoding the polypeptides and/or nucleic acid component(s), and wherein the one or more polynucleotides are operably configured to express the IsrB polypeptide and/or the ωRNA molecule,
and optionally wherein contacting comprises cleaving a DNA polynucleotide; or contacting results in modification of a gene product or modification of an amount or expression of a gene product.

17-19. (canceled)

20. An engineered, non-naturally occurring composition comprising:
a. a IsrB polypeptide, wherein the IsrB polypeptide is catalytically inactive,
b. a nucleotide deaminase (e.g., adenosine deaminase or a cytidine deaminase) associated with or otherwise capable of forming a complex with the IsrB polypeptide, and
c. an ωRNA molecule capable of forming a complex with the IsrB polypeptide and directing site-specific binding at a target sequence.

21. (canceled)

22. One or more polynucleotides encoding one or more components of the composition of claim 20.

23. One or more vectors encoding the one or more polynucleotides of claim 22.

24. A cell or progeny thereof genetically engineered to express one or more components of the composition of claim 20.

25. A method of editing nucleic acids in target polynucleotides comprising delivering the composition of claim 20 to a cell or population of cells comprising target polynucleotides,
and optionally, wherein the target polynucleotides are target sequences within genomic DNA; and/or wherein the target polynucleotide is edited at one or more bases to introduce a G→A or C→T mutation.

26. (canceled)

27. (canceled)

28. An isolated cell or progeny thereof comprising one or more base edits made using the method of claim 25.

29. An engineered, non-naturally occurring composition comprising:
a. a IsrB polypeptide, wherein the IsrB polypeptide catalytically inactive,
b. a reverse transcriptase associated with or otherwise capable of forming a complex with the IsrB polypeptide, and
c. ωRNA molecule capable of forming a complex with the IsrB polypeptide and directing site-specific binding of the complex to a target sequence of a target polynucleotide, and further comprising a donor template encoding a donor sequence for insertion into the target polynucleotide.

30. One or more polynucleotides encoding one or more components of the composition of claim 29.

31. One or more vectors encoding the one or more polynucleotides of claim 30.

32. A method of modifying target polynucleotides comprising
delivering the composition of claim 29 to a cell or population of cells comprising the target polynucleotides, wherein the complex directs the reverse transcriptase to the target sequence and the reverse transcriptase facilitates insertion of the donor sequence from the ωRNA molecule into the target polynucleotide; and optionally wherein insertion of the donor sequence:
a. introduces one or more base edits;
b. corrects or introduces a premature stop codon;
c. disrupts a splice site;
d. inserts or restores a splice site;
e. inserts a gene or gene fragment at one or both alleles of the target polynucleotide; or;
f. a combination thereof.

33. (canceled)

34. An isolated cell or progeny thereof comprising modifying the isolated cell or progeny thereof using the method of claim 32.

35. An engineered, non-naturally occurring composition comprising:
a. a catalytically inactive IsrB polypeptide;
b a non-LTR retrotransposon protein or integrase associated with or otherwise capable of forming a complex with the catalytically inactive IsrB polypeptide;
c. ωRNA molecule capable of forming a complex with the IsrB polypeptide and directing site-specific binding to a target sequence of a target polynucleotide; and
d. a donor construct comprising a donor polynucleotide for insertion to the target polynucleotide and located between two binding elements capable of forming a complex with the non-LTR retrotransposon protein or integrase,
and, optionally, wherein the catalytically inactive IsrB polypeptide is fused to an N-terminus of the non-LTR retrotransposon protein or integrase; and/or wherein the catalytically inactive IsrB polypeptide has nickase activity,
and optionally, wherein the donor polynucleotide further comprises a polymerase processing element to facilitate 3′ end processing of the donor polynucleotide sequence; or wherein the donor polynucleotide further comprises a homology region to the target sequence on a 5′ end of the donor construct, a 3′ end of the donor construct, or both.

36-39. (canceled)

37. (canceled)

39. (canceled)

40. One or more polynucleotides encoding one or more components of the composition of claim 35.

41. One or more vectors comprising the one or more polynucleotides of claim 40.

42. A method of modifying target polynucleotides comprising
delivering the composition of claim 35 to a cell or population of cells comprising the target polynucleotides, wherein the complex directs the non-LTR retrotransposon protein to the target sequence and the non-LTR retrotransposon protein facilitates insertion of the donor polynucleotide sequence from the donor construct into the target polynucleotide, and optionally, wherein insertion of the donor sequence:
a. introduces one or more base edits;
b. corrects or introduces a premature stop codon;
c. disrupts a splice site;
d. inserts or restores a splice site;
e. inserts a gene or gene fragment at one or both alleles of the target polynucleotide; or;
f. a combination thereof.

43. (canceled)

44. An isolated cell or progeny thereof comprising modifying the isolated cell or progeny thereof using the method of claim 42.

45. (canceled)

46. An engineered, non-naturally occurring composition comprising:
a. the IsrB polypeptide of claim 1,
b. a non-LTR retrotransposon protein or integrase associated with or otherwise capable of forming a complex with the IsrB polypeptide;
c. a ωRNA molecule capable of forming a complex with the IsrB polypeptide and directing site-specific binding to a target sequence of a target polynucleotide; and
d. a donor construct comprising a donor polynucleotide for insertion to the target polynucleotide and located between two binding elements capable of forming a complex with the non-LTR retrotransposon protein or integrase,
and optionally, wherein the IsrB protein is fused to an N-terminus of the non-LTR retrotransposon protein or integrase; and/or wherein the IsrB protein has nickase activity,
and optionally, wherein the donor polynucleotide further comprises a polymerase processing element to facilitate 3′ end processing of the donor polynucleotide sequence; or
wherein the donor polynucleotide further comprises a homology region to the target sequence on a 5′ end of the donor construct, a 3′ end of the donor construct, or both.

47-50. (canceled)

51. One or more polynucleotides encoding one or more components of the composition of claim 35.

52. One or more vectors comprising the one or more polynucleotides of claim 51.

53. A method of modifying target polynucleotides comprising
delivering the composition of claim 35 to a cell or population of cells comprising the target polynucleotides, wherein the complex directs the non-LTR retrotransposon protein to the target sequence and the non-LTR retrotransposon protein facilitates insertion of the donor polynucleotide sequence from the donor construct into the target polynucleotide, and optionally, wherein insertion of the donor sequence:
a. introduces one or more base edits;
b. corrects or introduces a premature stop codon;
c. disrupts a splice site;
d. inserts or restores a splice site;
e. inserts a gene or gene fragment at one or both alleles of the target polynucleotide; or;
f. a combination thereof.

54. (canceled)

55. An isolated cell or progeny thereof comprising modifying the isolated cell or progeny thereof using the method of claim 53.
